(19) United States
(12) Patent Application Publication
(10) Pub. No.: US 2014/0154691 A1
(43) Pub. Date: Jun. 5, 2014

(54) Methods and Compositions for the Treatment and Diagnosis of Bladder Cancer

(71) Applicants: Karen Chapman, Mill Valley, CA (US); Joseph Wagner, San Ramon, CA (US); Michael West, Mill Valley, CA (US); Markus Daniel Lacher, Lafayette, CA (US); Jennifer Lorie Kidd, Alameda, CA (US)

(72) Inventors: Karen Chapman, Mill Valley, CA (US); Joseph Wagner, San Ramon, CA (US); Michael West, Mill Valley, CA (US); Markus Daniel Lacher, Lafayette, CA (US); Jennifer Lorie Kidd, Alameda, CA (US)

(73) Assignee: ONCOCYTE CORPORATION, Alameda, CA (US)

(21) Appl. No.: 14/127,895

(22) PCT Filed: Jun. 22, 2012
(86) PCT No.: PCT/US2012/043852

§ 371 Date: Dec. 23, 2013

Related U.S. Application Data
(60) Provisional application No. 61/500,085, filed on Jun. 22, 2011.

(51) Int. Cl. C12Q 1/68; G01N 33/574

(52) U.S. Cl. CPC C12Q 1/6886; G01N 33/57407
USPC 435/6.12; 435/7.92

(57) ABSTRACT

Embodiments of the disclosure are directed to methods of diagnosis, prognosis and treatment of cancer. The methods are particularly suited for bladder cancer. The methods include targeting a marker that is expressed at abnormal levels in bladder cancer tissue in comparison to normal somatic tissue. Also disclosed are methods of treating cancer comprising administering a composition including a therapeutic that affects the expression or function of a target marker.

This application claims priority to U.S. Provisional Application Ser. No. 61/500,085 filed Jun. 22, 2011, the entire contents of which is hereby incorporated by reference.

### FIELD OF THE INVENTION

The field of the invention relates to cancer and the diagnosis and treatment of cancer.

### BACKGROUND

Bladder cancer is a type of malignant growths of the urinary bladder. The most common type of bladder cancer begins in cells lining the inside of the bladder and is called transitional cell carcinoma (sometimes urothelial cell carcinoma). Types of bladder cancers include transitional cell carcinoma, squamous cell carcinoma, adenocarcinoma, sarcoma, small cell carcinoma and secondary deposits from cancers elsewhere in the body. Bladder cancer characteristically causes blood in the urine; this may be visible to the naked eye (gross hematuria) or detectable only by microscope (microscopic hematuria). Other possible symptoms include pain during urination, frequent urination (polyuria) or feeling the need to urinate without results

The gold standard for diagnosing bladder cancer is biopsy obtained during cystoscopy. Sometimes it is an incidental finding during cystoscopy. Urine cytology can be obtained in voided urine or at the time of the cystoscopy (“bladder washing”). Cytology is very specific (a positive result is highly indicative of bladder cancer) but suffers from low sensitivity (inability of a negative result to reliably exclude bladder cancer). There are newer urine bound markers for the diagnosis of bladder cancer. These markers are not currently used routinely in clinical practice due to absence of clear professional guidelines. They are much more expensive as well. Bladder cancer may also be diagnosed with a Cysview™ guided fluorescence cystoscopy, as an adjunct to conventional white-light cystoscopy. This procedure improves the detection of bladder cancer and reduces the rate of early tumor recurrence, compared with white-light cystoscopy alone.

Many patients with a history, signs, and symptoms of bladder cancer are referred to a urologist or other physician trained in cystoscopy, a procedure in which a flexible tube bearing a camera and various instruments is introduced into the bladder through the urethra. Suspicious lesions may be biopsied and sent for pathologic analysis. These procedures are invasive.

There is a need in the field of cancer diagnostics for a highly specific, highly sensitive, rapid, inexpensive, and relatively non-invasive method of diagnosing bladder cancer. Various embodiments of the invention described below meet this need as well as other needs in the field of diagnosing and treating bladder cancer.

### SUMMARY OF THE INVENTION

Embodiments of the disclosure provide for methods of diagnosis, prognosis and treatment of bladder cancer.

In certain embodiments the invention provides a method of detecting bladder cancer in a subject comprising a) contacting a sample obtained from the subject with one or more agents that detect one or more markers expressed by a bladder cancer cell b) contacting a non-cancerous cell with the one or more agents from a); and c) comparing the expression level of the marker in the sample obtained from the subject with the expression level in the non-cancerous cell, wherein a higher level of expression of the marker in the sample compared to the non-cancerous cell indicates that the subject has bladder cancer.

In certain embodiments the invention provides a method of detecting bladder cancer in a subject comprising a) contacting a sample obtained from the subject with one or more agents that detect expression of at least one of the markers listed in Table 2 or 3; b) contacting a non-cancerous cell, e.g., a non-cancerous cell from bladder tissue or a noncancerous bladder cell line, with the one or more agents from a); and c) comparing the expression level of one or more of the markers listed in Table 2 or 3 in the sample obtained from the subject with the expression level of one or more of the markers listed in Table 2 or 3 in the non-cancerous cell, wherein a higher level of expression of one or more of the markers listed in Table 2 or 3 in the sample compared to the non-cancerous cell indicates that the subject has bladder cancer.

In some embodiments the invention provides a method of detecting bladder cancer in a subject comprising a) contacting a sample obtained from the subject with one or more agents that detect expression of at least one of the markers chosen from MAGEA10, DSCR8, MMP12, CXCL9, DSCR8, KRT81, LOC729826, PTHLH, MMP11, S100A7, WISP3, CXCL10, NMU, GBP5, TOP2A, SERPINB4, GNLY, GTSF1, PI3, S100A7A, IDO1, GJB6, CALML3, SERPINB3, CXCL6, OLFM4, TCN1, VSNL1, UBD, AIM2, ABCC9, SERPINB13, INDO, KRT5, LOC100130897, KRT14, FAM83A, FAM181B, GZMB, DSG3, TYMP, KRT6A, KRT6B, HLA-DRB1, LCN2, KRT4, IFI30, LOC100134370, KIAA1618, S100A8, MMP7, MMP7, SPRR2A, GJB2, COL10A1, FCRLB, SFN, S100A2, IL1A, KRT16 SLC1A6, and SERPINB5; b) contacting a non-cancerous cell, e.g. a non-cancerous cell from bladder tissue or a non-cancerous bladder cell line, with the one or more agents that detect expression of at least one of the markers chosen from MAGEA10, DSCR8, MMP12, CXCL9, DSCR8, KRT81, LOC729826, PTHLH, MMP11, S100A7, WISP3, CXCL10, NMU, GBP5, TOP2A, SERPINB4, GNLY, GTSF1, PI3, S100A7A, IDO1, GJB6, CALML3, SERPINB3, CXCL6, OLFM4, TCN1, VSNL1, UBD, AIM2, ABCC9, SERPINB13, INDO, KRT5, LOC100130897, KRT14, FAM83A, FAM181B, GZMB, DSG3, TYMP, KRT6A, KRT6B, HLA-DRB1, LCN2, KRT4, IFI30, LOC100134370, KIAA1618, S100A8, MMP7, MMP7, SPRR2A, GJB2, COL10A1, FCRLB, SFN, S100A2 IL1A, KRT16 SLC1A6, and SERPINB5; and c) comparing the expression level of one or more of the markers chosen from MAGEA10, DSCR8, MMP12, CXCL9, DSCR8, KRT81, LOC729826, PTHLH, MMP11, S100A7, WISP3, CXCL10, NMU, GBP5, TOP2A, SERPINB4, GNLY, GTSF1, PI3, S100A7A, IDO1, GJB6, CALML3, SERPINB3, CXCL6, OLFM4, TCN1, VSNL1, UBD, AIM2, ABCC9, SERPINB13, INDO, KRT5, LOC100130897, KRT14, FAM83A, FAM181B, GZMB, DSG3, TYMP, KRT6A, KRT6B, HLA-DRB1, LCN2, KRT4, IFI30, LOC100134370, KIAA1618, S100A8, MMP7, MMP7, SPRR2A, GJB2, COL10A1, FCRLB, SFN, S100A IL1A, KRT16 SLC1A6, and SERPINB52 in the sample obtained from the subject with the expression level of one or more of the markers chosen from MAGEA10, DSCR8, MMP12, CXCL9, DSCR8, KRT81, LOC729826, PTHLH, MMP11, S100A7, WISP3, CXCL10, NMU, GBP5, TOP2A, SERPINB4, GNLY, GTSF1, PI3, S100A7A, IDO1, GJB6, CALML3, SERPINB3, CXCL6, OLFM4, TCN1, VSNL1, UBD, AIM2, ABCC9, SERPINB13, INDO, KRT5, LOC100130897, KRT14, FAM83A, FAM181B, GZMB, DSG3, TYMP, KRT6A, KRT6B, HLA-DRB1, LCN2, KRT4, IFI30, LOC100134370, KIAA1618, S100A8, MMP7, MMP7, SPRR2A, GJB2, COL10A1, FCRLB, SFN, S100A2 IL1A, KRT16 SLC1A6, and SERPINB5 in the non-cancerous cell, wherein a higher level of expression of one or more of the markers chosen from MAGEA10, DSCR8, MMP12, CXCL9, DSCR8, KRT81, LOC729826, PTHLH, MMP11, S100A7, WISP3, CXCL10, NMU, GBP5, TOP2A, SERPINB4, GNLY, GTSF1, PI3, S100A7A, IDO1, GJB6, CALML3, SERPINB3, CXCL6, OLFM4, TCN1, VSNL1, UBD, AIM2, ABCC9, SERPINB13, INDO, KRT5, LOC100130897, KRT14, FAM83A, FAM181B, GZMB, DSG3, TYMP, KRT6A, KRT6B, HLA-DRB1, LCN2, KRT4, IFI30, LOC100134370, KIAA1618, S100A8, MMP7, MMP7, SPRR2A, GJB2, COL10A1, FCRLB, SFN, S100A2 IL1A, KRT16 SLC1A6, and SERPINB5 in the sample compared to the non-cancerous cell indicates that the subject has bladder cancer.

With regard to the embodiments described in the preceding paragraphs, the sample may be any sample as described infra, for example, a bodily fluid, such as blood, serum or urine. The sample may be a cellular sample, a tissue sample or the extract of a cellular or tissue sample. The agent may be one or more molecules that bind specifically to one or more proteins expressed by the cancer cell or one or more nucleic acids expressed by the cell. For example, the agent may be a protein such as an antibody that binds specifically to the protein expressed by one of the marker genes identified infra. The agent may be one or more nucleic acids that hybridize to a nucleic acid expressed by the cancer cell. The nucleic acid expressed by the cancer cell may be an RNA molecule, e.g. an mRNA molecule. The nucleic acid molecule that hybridizes to the nucleic acid expressed by the cancer cell may be a DNA molecule, such as a DNA probe.

In still other embodiments the invention provides a composition of matter useful in distinguishing a bladder cancer cell from a non-cancerous cell comprising one or more molecules that specifically bind to a molecule expressed at higher levels on a bladder cancer cell compared to a non-cancer cell. As an example, the composition may comprise a protein, that binds to one or more molecules expressed by the cancer cell at higher levels compared to the non-cancer cell. As another example, the composition may comprise a nucleic acid that binds to one or more molecules expressed by the bladder cancer cell at higher levels compared to the non-cancer cell.

In some embodiments the invention provides a composition of matter comprising a protein, such as an antibody, that specifically binds to a molecule expressed by a bladder cancer cell chosen from the markers listed in Table 3. The molecule expressed by the bladder cancer cell may be expressed by the bladder cancer cell at level that is higher than the level expressed by a non-cancerous cell such as a non-cancerous bladder tissue cell or non-cancerous bladder cell line.

In certain embodiments the invention provides a composition of matter comprising a protein, such as an antibody, that specifically binds to a molecule expressed by a bladder cancer cell chosen from MAGEA10, DSCR8, MMP12, CXCL9, DSCR8, KRT81, LOC729826, PTHLH, MMP11, S100A7, WISP3, CXCL10, NMU, GBP5, TOP2A, SERPINB4, GNLY, GTSF1, PI3, S100A7A, IDOL GJB6, CALML3, SERPINB3, CXCL6, OLFM4, TCN1, VSNL1, UBD, AIM2, ABCC9, SERPINB13, INDO, KRT5, LOC100130897, KRT14, FAM83A, FAM181B, GZMB, DSG3, TYMP, KRT6A, KRT6B, HLA-DRB1, LCN2, KRT4, IFI30, LOC100134370, KIAA1618, S100A8, MMP7, MMP7, SPRR2A, GJB2, COL10A1, FCRLB, SFN, S100A2 IL1A, KRT16 SLC1A6, and SERPINB5. The molecule expressed by the bladder cancer cell may be expressed by the bladder cancer cell at level that is higher than the level expressed by a non-cancerous cell such as a non-cancerous bladder tissue cell.

In other embodiments the invention provides a composition of matter comprising a nucleic acid that specifically binds to a molecule, such as an mRNA molecule, expressed by a bladder cancer cell wherein the molecule is chosen from a marker listed in Table 1 or 2. The molecule expressed by the bladder cancer cell may be expressed by the bladder cancer cell at level that is higher than the level expressed by a non-cancerous cell such as a non-cancerous bladder tissue cell.

In other embodiments the invention provides a composition of matter comprising a nucleic acid that specifically binds to a molecule, such as an mRNA molecule, expressed by a bladder cancer cell wherein the molecule is chosen from a nucleic acid encoding MAGEA10, DSCR8, MMP12, CXCL9, DSCR8, KRT8I, LOC729826, PTHLH, MMP11, S100A7, WISP3, CXCL10, NMU, GBP5, TOP2A, SERPINB4, GNLY, GTSF1, PI3, S100A7A, IDO1, GJB6, CALML3, SERPINB3, CXCL6, OLFM4, TCN1, VSNL1, UBD, AIM2, ABCC9, SERPINB13, INDO, KRT5, LOC100130897, KRT14, FAM83A, FAM181B, GZMB, DSG3, TYMP, KRT6A, KRT6B, HLA-DRB1, LCN2, KRT4, IFI30, LOC100134370, KIAA1618, S100A8, MMP7, MMP7, SPRR2A, GJB2, COL10A1, FCRLB, SFN, S100A2 IL1A, KRT16 SLC1A6, and SERPINB5. The molecule expressed by the bladder cancer cell may be expressed by the bladder cancer cell at level that is higher than the level expressed by a non-cancerous cell such as a non-cancerous bladder tissue cell.

In still further embodiments the invention provides a method of determining if a cancer in a subject is advancing comprising a) measuring the expression level of one or more markers associated with cancer at a first time point; b) measuring the expression level of the one or more markers measured in a) at a second time point, wherein the second time point is subsequent to the first time point; and c) comparing the expression level measured in a) and b), wherein an increase in the expression level of the one or more markers in b) compared to a) indicates that the subject's cancer is advancing. In certain embodiments the cancer is bladder cancer.

In some embodiments the invention provides a method of determining if a bladder cancer in a subject is advancing comprising a) measuring the expression level of one or more markers listed in Table 2 or 3 at a first time point; b) measuring the expression level of the one or more markers measured in a) at a second time point, wherein the second time point is subsequent to the first time point; and c) comparing the expression level measured in a) and b), wherein an increase in the expression level of the one or more markers at the second time point compared to the first time point indicates that the subject's bladder cancer is advancing.

In other embodiments the invention provides a method of determining if a bladder cancer in a subject is advancing comprising a) measuring the expression level of one or more markers chosen from MAGEA10, DSCR8, MMP12, CXCL9, DSCR8, KRT81, LOC729826, PTHLH, MMP11, S100A7, WISP3, CXCL10, NMU, GBP5, TOP2A, SERPINB4, GNLY, GTSF1, PI3, S100A7A, IDO1, GJB6, CALML3, SERPINB3, CXCL6, OLFM4, TCN1, VSNL1, UBD, AIM2, ABCC9, SERPINB13, INDO, KRT5, LOC100130897, KRT14, FAM83A, FAM181B, GZMB, DSG3, TYMP, KRT6A, KRT6B, HLA-DRB1, LCN2, KRT4, IFI30, LOC100134370, KIAA1618, S100A8, MMP7, MMP7, SPRR2A, GJB2, COL10A1, FCRLB, SFN, S100A2 IL1A, KRT16 SLC1A6, and SERPINB5 at a first time point; b) measuring the expression level of the one or more markers measured in a) at a second time point, wherein the second time point is subsequent to the first time point; and c) comparing the expression level measured in a) and b), wherein an increase in the expression level of the one or more markers at the second time point compared to the first time point indicates that the subject's bladder cancer is advancing.

In some embodiments the invention provides antigens (i.e. cancer-associated polypeptides) associated with bladder cancer as targets for diagnostic and/or therapeutic antibodies. In some embodiments, the antigen may be chosen from a protein encoded by, a gene listed in Table 2, a fragment thereof, or a combination of proteins encoded by a gene listed in Table 2.

In some embodiments the invention provides antigens (i.e. cancer-associated polypeptides) associated with bladder cancer as targets for diagnostic and/or therapeutic antibodies. In some embodiments, the antigen may be chosen from a protein encoded by, a gene chosen from MAGEA10, DSCR8, MMP12, CXCL9, DSCR8, KRT81, LOC729826, PTHLH, MMP11, S100A7, WISP3, CXCL10, NMU, GBP5, TOP2A, SERPINB4, GNLY, GTSF1, PI3, S100A7A, IDO1, GJB6, CALML3, SERPINB3, CXCL6, OLFM4, TCN1, VSNL1, UBD, AIM2, ABCC9, SERPINB13, INDO, KRT5, LOC100130897, KRT14, FAM83A, FAM181B, GZMB, DSG3, TYMP, KRT6A, KRT6B, HLA-DRB1, LCN2, KRT4, IFI130, LOC100134370, KIAA1618, S100A8, MMP7, MMP7, SPRR2A, GJB2, COL10A1, FCRLB, SFN, S100A2 IL1A, KRT16 SLC1A6, and SERPINB5, a fragment thereof, or a combination of proteins encoded by a gene chosen from MAGEA10, DSCR8, MMP12, CXCL9, DSCR8, KRT81, LOC729826, PTHLH, MMP11, S100A7, WISP3, CXCL10, NMU, GBP5, TOP2A, SERPINB4, GNLY, GTSF1, PI3, S100A7A, IDO1, GJB6, CALML3, SERPINB3, CXCL6, OLFM4, TCN1, VSNL1, UBD, AIM2, ABCC9, SERPINB13, INDO, KRT5, LOC100130897, KRT14, FAM83A, FAM181B, GZMB, DSG3, TYMP, KRT6A, KRT6B, HLA-DRB1, LCN2, KRT4, IFI30, LOC100134370, KIAA1618, S100A8, MMP7, MMP7, SPRR2A, GJB2, COL10A1, FCRLB, SFN, S100A2 IL1A, KRT16 SLC1A6, and SERPINB5.

In yet other embodiments the invention provides a method of eliciting an immune response to a bladder cancer cell comprising contacting a subject with a protein or protein fragment that is expressed by a bladder cancer cell thereby eliciting an immune response to the cancer cell. As an example the subject may be contacted intravenously or intramuscularly.

In further embodiments the invention provides a method of eliciting an immune response to a bladder cancer cell comprising contacting a subject with one or more proteins or protein fragments that is encoded by a gene chosen from the genes listed in Table 3, thereby eliciting an immune response to a bladder cancer cell. As an example the subject may be contacted intravenously or intramuscularly.

In still other embodiments the invention provides a method of eliciting an immune response to a bladder cancer cell comprising contacting a subject with one or more proteins or protein fragments that is encoded by a gene chosen from MAGEA10, DSCR8, MMP12, CXCL9, DSCR8, KRT81, LOC729826, PTHLH, MMP11, S100A7, WISP3, CXCL10, NMU, GBP5, TOP2A, SERPINB4, GNLY, GTSF1, PI3, S100A7A, IDO1, GJB6, CALML3, SERPINB3, CXCL6, OLFM4, TCN1, VSNL1, UBD, AIM2, ABCC9, SERPINB13, INDO, KRT5, LOC100130897, KRT14, FAM83A, FAM181B, GZMB, DSG3, TYMP, KRT6A, KRT6B, HLA-DRB1, LCN2, KRT4, IFI30, LOC100134370, KIAA1618, S100A8, MMP7, MMP7, SPRR2A, GJB2, COL10A1, FCRLB, SFN, S100A2 IL1A, KRT16 SLC1A6, and SERPINB5, thereby eliciting an immune response to a bladder cancer cell. As an example the subject may be contacted intravenously or intramuscularly.

In other embodiments the invention provides a kit for detection of cancer in a sample obtained from a subject. The kit may comprise one or more agents that bind specifically to a molecule expressed by a bladder cancer cell. The molecule may be expressed at a higher level in the bladder cancer cell compared to a non-cancerous cell, such as a non-cancerous bladder cell. The kit may comprise one or more containers and instructions for determining if the sample is positive for cancer. The kit may optionally contain one or more multiwell plates, a detectable substance or label such as a dye, a radioactively labeled molecule, a chemiluminescently labeled molecule and the like. The kit may further contain a positive control (e.g. one or more cancerous bladder cells; or specific known quantities of the molecule expressed by the cancer cell) and a negative control (e.g. a tissue or cell sample that is non-cancerous).

In some embodiments the invention provides a kit for the detection of bladder cancer comprising one or more agents that specifically bind one or more markers chosen from MAGEA10, DSCR8, MMP12, CXCL9, DSCR8, KRT81, LOC729826, PTHLH, MMP11, S100A7, WISP3, CXCL10, NMU, GBP5, TOP2A, SERPINB4, GNLY, GTSF1, PI3, S100A7A, IDO1, GJB6, CALML3, SERPINB3, CXCL6, OLFM4, TCN1, VSNL1, UBD, AIM2, ABCC9, SERPINB13, INDO, KRT5, LOC100130897, KRT14, FAM83A, FAM181B, GZMB, DSG3, TYMP, KRT6A, KRT6B, HLA-DRB1, LCN2, KRT4, IFI30, LOC100134370, KIAA1618, S100A8, MMP7, MMP7, SPRR2A, GJB2, COL10A1, FCRLB, SFN, S100A2 IL1A, KRT16 SLC1A6, and SERPINB5. The kit may comprise one or more containers and instructions for determining if the sample is positive for cancer. The kit may optionally contain one or more multiwell plates, a detectable substance such as a dye, a radioactively labeled molecule, a chemiluminescently labeled molecule and the like. The kit may further contain a positive control (e.g. one or more cancerous cells; or specific known quantities of the molecule expressed by the cancer cell) and a negative control (e.g. a tissue or cell sample that is non-cancerous). As an example the kit may take the form of an ELISA or a DNA microarray.

Some embodiments herein are directed to a method of treating bladder cancer in a subject, the method comprising administering to a subject in need thereof a therapeutic agent capable of modulating the activity of a cancer associated protein, wherein the cancer associated protein is encoded by gene listed in Table 2, homologs thereof, combinations thereof, or a fragment thereof. In some embodiments, the therapeutic agent binds to the bladder cancer associated protein. In some embodiments, the therapeutic agent is an antibody. In some embodiments, the antibody may be a monoclonal antibody or a polyclonal antibody. In some embodiments, the antibody is a humanized or human antibody.

Some embodiments herein are directed to a method of treating bladder cancer in a subject, the method comprising administering to a subject in need thereof a therapeutic agent modulating the activity of a cancer associated protein, wherein the cancer associated protein is encoded by gene chosen from MAGEA10, DSCR8, MMP12, CXCL9, DSCR8, KRT81, LOC729826, PTHLH, MMP11, S100A7, WISP3, CXCL10, NMU, GBP5, TOP2A, SERPINB4, GNLY, GTSF1, PI3, S100A7A, IDO1, GJB6, CALML3, SERPINB3, CXCL6, OLFM4, TCN1, VSNL1, UBD, AIM2, ABCC9, SERPINB13, INDO, KRT5, LOC100130897, KRT14, FAM83A, FAM181B, GZMB, DSG3, TYMP, KRT6A, KRT6B, HLA-DRB1, LCN2, KRT4, IFI130, LOC100134370, KIAA1618, S100A8, MMP7, MMP7, SPRR2A, GJB2, COL10A1, FCRLB, SFN, S100A2 IL1A, KRT16 SLC1A6, and SERPINB5, homologs thereof, combinations thereof, or a fragment thereof. In some embodiments, the therapeutic agent binds to the bladder cancer associated protein. In some embodiments, the therapeutic agent is an antibody. In some embodiments, the antibody may be a monoclonal antibody or a polyclonal antibody. In some embodiments, the antibody is a humanized or human antibody.

In some embodiments, a method of treating bladder cancer in a subject may comprise administering to a subject in need thereof a therapeutic agent that modulates the activity of one or more genes chosen from those listed in Table 2.

In some embodiments, a method of treating bladder cancer in a subject may comprise administering to a subject in need thereof a therapeutic agent that modulates the activity of one or more genes chosen from MAGEA10, DSCR8, MMP12, CXCL9, DSCR8, KRT81, LOC729826, PTHLH, MMP11, S100A7, WISP3, CXCL10, NMU, GBP5, TOP2A, SERPINB4, GNLY, GTSF1, PI3, S100A7A, IDO1, GJB6, CALML3, SERPINB3, CXCL6, OLFM4, TCN1, VSNL1, UBD, AIM2, ABCC9, SERPINB13, INDO, KRT5, LOC100130897, KRT14, FAM83A, FAM181B, GZMB, DSG3, TYMP, KRT6A, KRT6B, HLA-DRB1, LCN2, KRT4, IFI30, LOC100134370, KIAA1618, S100A8, MMP7, MMP7, SPRR2A, GJB2, COL10A1, FCRLB, SFN, S100A2 IL1A, KRT16 SLC1A6, and SERPINB5.

In further embodiments, the invention provides a method of treating bladder cancer may comprise a gene knockdown of one or more genes listed in Table 2. In some embodiments, a method of treating bladder cancer may comprise treating cells to knockdown or inhibit expression of a gene encoding an mRNA of one or more genes chosen from those listed n Table 2.

In other embodiments, a method of treating bladder cancer may comprise gene knockdown of one or more genes selected from MAGEA10, DSCR8, MMP12, CXCL9, DSCR8, KRT81, LOC729826, PTHLH, MMP11, S100A7, WISP3, CXCL10, NMU, GBP5, TOP2A, SERPINB4, GNLY, GTSF1, PI3, S100A7A, IDO1, GJB6, CALML3, SERPINB3, CXCL6, OLFM4, TCN1, VSNL1, UBD, AIM2, ABCC9, SERPINB13, INDO, KRT5, LOC100130897, KRT14, FAM83A, FAM181B, GZMB, DSG3, TYMP, KRT6A, KRT6B, HLA-DRB1, LCN2, KRT4, IFI30, LOC100134370, KIAA1618, S100A8, MMP7, MMP7, SPRR2A, GJB2, COL10A1, FCRLB, SFN, S100A2 IL1A, KRT16 SLC1A6, and SERPINB5. In some embodiments, a method of treating bladder cancer may comprise treating cells to knockdown or inhibit expression of a gene encoding an mRNA of one or more genes chosen from MAGEA10, DSCR8, MMP12, CXCL9, DSCR8, KRT81, LOC729826, PTHLH, MMP11, S100A7, WISP3, CXCL10, NMU, GBP5, TOP2A, SERPINB4, GNLY, GTSF1, PI3, S100A7A, IDO1, GJB6, CALML3, SERPINB3, CXCL6, OLFM4, TCN1, VSNL1, UBD, AIM2, ABCC9, SERPINB13, INDO, KRT5, LOC100130897, KRT14, FAM83A, FAM181B, GZMB, DSG3, TYMP, KRT6A, KRT6B, HLA-DRB1, LCN2, KRT4, IFI30, LOC100134370, KIAA1618, S100A8, MMP7, MMP7, SPRR2A, GJB2, COL10A1, FCRLB, SFN, S100A2 IL1A, KRT16 SLC1A6, and SERPINB5

In still other embodiments, the present invention provides methods of screening a drug candidate for activity against bladder cancer, the method comprising: (a) contacting a cell that expresses one or more cancer associated genes chosen from those listed in Table 2 with a drug candidate; (b) detecting an effect of the drug candidate on an expression of the one or more bladder cancer associated genes in the cell from a); and (c) comparing the level of expression of one or more of the genes recited in a) in the absence of the drug candidate to the level of expression of the one or more genes in the presence of the drug candidate; wherein a decrease in the expression of the bladder cancer associated gene in the presence of the drug candidate indicates that the candidate has activity against bladder cancer.

In further embodiments, the present invention provides methods of screening a drug candidate for activity against bladder cancer, the method comprising: (a) contacting a cell that expresses one or more bladder cancer associated genes chosen from MAGEA10, DSCR8, MMP12, CXCL9, DSCR8, KRT81, LOC729826, PTHLH, MMP11, S100A7, WISP3, CXCL10, NMU, GBP5, TOP2A, SERPINB4, GNLY, GTSF1, PI3, S100A7A, IDO1, GJB6, CALML3, SERPINB3, CXCL6, OLFM4, TCN1, VSNL1, UBD, AIM2, ABCC9, SERPINB13, INDO, KRT5, LOC100130897, KRT14, FAM83A, FAM181B, GZMB, DSG3, TYMP, KRT6A, KRT6B, HLA-DRB1, LCN2, KRT4, IFI30, LOC100134370, KIAA1618, S 100A8, MMP7, MMP7, SPRR2A, GJB2, COL10A1, FCRLB, SFN, S100A2 IL1A, KRT16 SLC1A6, and SERPINB5 with a drug candidate; (b) detecting an effect of the drug candidate on an expression of the one or more bladder cancer associated genes in the cell from a); and (c) comparing the level of expression of one or more of the genes recited in a) in the absence of the drug candidate to the level of expression in the presence of the drug candidate; wherein a decrease in the expression of the bladder cancer associated gene in the presence of the drug candidate indicates that the candidate has activity against bladder cancer.

In some embodiments, the present invention provides methods of visualizing a bladder cancer tumor in a subject comprising a) targeting one or more bladder cancer associated proteins with a labeled molecule that binds specifically to the bladder cancer tumor, wherein the cancer associated protein is selected from a protein encoded for by one or more genes chosen from those listed in Table 2; and b) detecting the labeled molecule, wherein the labeled molecule visualizes the tumor in the subject.

In still other embodiments, the present invention provides methods of visualizing a bladder cancer tumor in a subject comprising a) targeting one or more bladder cancer associated proteins with a labeled molecule that binds specifically to the bladder cancer associated protein, wherein the cancer associated protein is selected from a protein encoded for by one or more genes chosen from MAGEA10, DSCR8, MMP12, CXCL9, DSCR8, KRT81, LOC729826, PTHLH, MMP11, S100A7, WISP3, CXCL10, NMU, GBP5, TOP2A, SERPINB4, GNLY, GTSF1, PI3, S100A7A, IDO1, GJB6, CALML3, SERPINB3, CXCL6, OLFM4, TCN1, VSNL1, UBD, AIM2, ABCC9, SERPINB13, INDO, KRT5, LOC100130897, KRT14, FAM83A, FAM181B, GZMB, DSG3, TYMP, KRT6A, KRT6B, HLA-DRB1, LCN2, KRT4, IFI30, LOC100134370, KIAA1618, S100A8, MMP7, MMP7, SPRR2A, GJB2, COL10A1, FCRLB, SFN, S100A2 IL1A, KRT16 SLC1A6, and SERPINB5; and b) detecting the labeled molecule, wherein the labeled molecule visualizes the tumor in the subject.

The invention also provides the use of one or more of the markers disclosed infra in the detection of bladder cancer in a subject.

The invention also provides the use of one or more of the markers disclosed infra in estimating the risk of morbidity of bladder cancer in a subject.

### DESCRIPTION OF DRAWINGS

For a fuller understanding of the nature and advantages of the present invention, reference should be had to the following detailed description taken in connection with the accompanying drawings, in which:

FIG. 1 is a chart of the microarray analysis data showing expression of all mRNA probe sequences in normal somatic cells, normal tissues, malignant tumors, and cancer cell lines.

FIGS. 2A-2L is a chart containing the sequence information for the cancer associated sequences including the sequences of the probes used to detect the gene sequences.

FIG. 3 shows the expression of the MAGEA10 mRNA (SEQ ID NO: 111) in normal somatic cells, normal tissues, malignant tumors, and cancer cell lines.

FIG. 4 shows the expression of the DSCR8 mRNA (SEQ ID NO: 112) in normal somatic cells, normal tissues, malignant tumors, and cancer cell lines.

FIG. 5 shows the expression of the MMP12 in RNA (SEQ ID NO: 113) in normal somatic cells, normal tissues, malignant tumors, and cancer cell lines.

FIG. 6 shows the expression of the CXCL9 mRNA (SEQ ID NO: 114) in normal somatic cells, normal tissues, malignant tumors, and cancer cell lines.

FIG. 7 shows the expression of the DSCR8 mRNA (SEQ ID NO: 115) in normal somatic cells, normal tissues, malignant tumors, and cancer cell lines.

FIG. 8 shows the expression of the KRT81 mRNA (SEQ ID NO: 116) in normal somatic cells, normal tissues, malignant tumors, and cancer cell lines.

FIG. 9 shows the expression of the LOC729826 mRNA (SEQ ID NO: 117) in normal somatic cells, normal tissues, malignant tumors, and cancer cell lines.

FIG. 10 shows the expression of the PTHLH mRNA (SEQ ID NO: 118) in normal somatic cells, normal tissues, malignant tumors, and cancer cell lines.

FIG. 11 shows the expression of the MMP11 mRNA (SEQ ID NO: 119) in normal somatic cells, normal tissues, malignant tumors, and cancer cell lines.

FIG. 12 shows the expression of the S100A7 mRNA (SEQ ID NO: 120) in normal somatic cells, normal tissues, malignant tumors, and cancer cell lines.

FIG. 13 shows the expression of the WISP3 mRNA (SEQ ID NO: 121) in normal somatic cells, normal tissues, malignant tumors, and cancer cell lines.

FIG. 14 shows the expression of the CXCL10 mRNA (SEQ ID NO: 122) in normal somatic cells, normal tissues, malignant tumors, and cancer cell lines.

FIG. 15 shows the expression of the NMU mRNA (SEQ ID NO: 123) in normal somatic cells, normal tissues, malignant tumors, and cancer cell lines.

FIG. 16 shows the expression of the GBP5 mRNA (SEQ ID NO: 124) in normal somatic cells, normal tissues, malignant tumors, and cancer cell lines.

FIG. 17 shows the expression of the TOP2A mRNA (SEQ ID NO: 125) in normal somatic cells, normal tissues, malignant tumors, and cancer cell lines.

FIG. 18 shows the expression of the SERPINB4 mRNA (SEQ ID NO: 126) in normal somatic cells, normal tissues, malignant tumors, and cancer cell lines.

FIG. 19 shows the expression of the GLNY mRNA (SEQ ID NO: 127) in normal somatic cells, normal tissues, malignant tumors, and cancer cell lines.

FIG. 20 shows the expression of the GTSF1 mRNA (SEQ ID NO: 128) in normal somatic cells, normal tissues, malignant tumors, and cancer cell lines.

FIG. 21 shows the expression of the PI3 mRNA (SEQ ID NO: 129) in normal somatic cells, normal tissues, malignant tumors, and cancer cell lines.

FIG. 22 shows the expression of the S100A7A mRNA (SEQ ID NO: 130) in normal somatic cells, normal tissues, malignant tumors, and cancer cell lines.

FIG. 23 shows the expression of the IDO1 mRNA (SEQ ID NO: 131) in normal somatic cells, normal tissues, malignant tumors, and cancer cell lines.

FIG. 24 shows the expression of the GJB6 mRNA (SEQ ID NO: 132) in normal somatic cells, normal tissues, malignant tumors, and cancer cell lines.

FIG. 25 shows the expression of the CALML3 mRNA (SEQ ID NO: 133) in normal somatic cells, normal tissues, malignant tumors, and cancer cell lines.

FIG. 26 shows the expression of the SERPINB3 mRNA (SEQ ID NO: 134) in normal somatic cells, normal tissues, malignant tumors, and cancer cell lines.

FIG. 27 shows the expression of the CXCL6 mRNA (SEQ ID NO: 135) in normal somatic cells, normal tissues, malignant tumors, and cancer cell lines.

FIG. 28 shows the expression of the OLFM4 mRNA (SEQ ID NO: 136) in normal somatic cells, normal tissues, malignant tumors, and cancer cell lines.

FIG. 29 shows the expression of the TCN1 mRNA (SEQ ID NO: 137) in normal somatic cells, normal tissues, malignant tumors, and cancer cell lines.

FIG. 30 shows the expression of the VSNL1 mRNA (SEQ ID NO: 138) in normal somatic cells, normal tissues, malignant tumors, and cancer cell lines.

FIG. 31 shows the expression of the UBD mRNA (SEQ ID NO: 139) in normal somatic cells, normal tissues, malignant tumors, and cancer cell lines.

FIG. 32 shows the expression of the AIM2 mRNA (SEQ ID NO: 140) in normal somatic cells, normal tissues, malignant tumors, and cancer cell lines.

FIG. 33 shows the expression of the ABCC9 mRNA (SEQ ID NO: 141) in normal somatic cells, normal tissues, malignant tumors, and cancer cell lines.

FIG. 34 shows the expression of the SERPINB13 mRNA (SEQ ID NO: 142) in normal somatic cells, normal tissues, malignant tumors, and cancer cell lines.

FIG. 35 shows the expression of the INDO mRNA (SEQ ID NO: 143) in normal somatic cells, normal tissues, malignant tumors, and cancer cell lines.

FIG. 36 shows the expression of the KRT5 mRNA (SEQ ID NO: 144) in normal somatic cells, normal tissues, malignant tumors, and cancer cell lines.

FIG. 37 shows the expression of the LOC100130897 mRNA (SEQ ID NO: 145) in normal somatic cells, normal tissues, malignant tumors, and cancer cell lines.

FIG. 38 shows the expression of the KRT14 mRNA (SEQ ID NO: 146) in normal somatic cells, normal tissues, malignant tumors, and cancer cell lines.

FIG. 39 shows the expression of the FAM83A mRNA (SEQ ID NO: 147) in normal somatic cells, normal tissues, malignant tumors, and cancer cell lines.

FIG. 40 shows the expression of the FAM181B mRNA (SEQ ID NO: 148) in normal somatic cells, normal tissues, malignant tumors, and cancer cell lines.

FIG. 41 shows the expression of the SEQ ID NO: 149 in normal somatic cells, normal tissues, malignant tumors, and cancer cell lines.

FIG. 42 shows the expression of the GZMB mRNA (SEQ ID NO: 150) in normal somatic cells, normal tissues, malignant tumors, and cancer cell lines.

FIG. 43 shows the expression of the DSG3 mRNA (SEQ ID NO: 151) in normal somatic cells, normal tissues, malignant tumors, and cancer cell lines.

FIG. 44 shows the expression of the TYMP mRNA (SEQ ID NO: 152) in normal somatic cells, normal tissues, malignant tumors, and cancer cell lines.

FIG. 45 shows the expression of the KRT6A mRNA (SEQ ID NO: 153) in normal somatic cells, normal tissues, malignant tumors, and cancer cell lines.

FIG. 46 shows the expression of the KRT6B mRNA (SEQ ID NO: 154) in normal somatic cells, normal tissues, malignant tumors, and cancer cell lines.

FIG. 47 shows the expression of the HLA-DRB1 mRNA (SEQ ID NO: 155) in normal somatic cells, normal tissues, malignant tumors, and cancer cell lines.

FIG. 48 shows the expression of the LCN2 mRNA (SEQ ID NO: 156) in normal somatic cells, normal tissues, malignant tumors, and cancer cell lines.

FIG. 49 shows the expression of the KRT4 mRNA (SEQ ID NO: 157) in normal somatic cells, normal tissues, malignant tumors, and cancer cell lines.

FIG. 50 shows the expression of the IFI30 mRNA (SEQ ID NO: 158) in normal somatic cells, normal tissues, malignant tumors, and cancer cell lines.

FIG. 51 shows the expression of the LOC100134370 mRNA (SEQ ID NO: 159) in normal somatic cells, normal tissues, malignant tumors, and cancer cell lines.

FIG. 52 shows the expression of the KIAA1618 mRNA (SEQ ID NO: 160) in normal somatic cells, normal tissues, malignant tumors, and cancer cell lines.

FIG. 53 shows the expression of the S100A8 mRNA (SEQ ID NO: 161) in normal somatic cells, normal tissues, malignant tumors, and cancer cell lines.

FIG. 54 shows the expression of the MMP7 mRNA (SEQ ID NO: 162) in normal somatic cells, normal tissues, malignant tumors, and cancer cell lines.

FIG. 55 shows the expression of the MMP7 mRNA (SEQ ID NO: 163) in normal somatic cells, normal tissues, malignant tumors, and cancer cell lines.

FIG. 56 shows the expression of the SPRR2A mRNA (SEQ ID NO: 164) in normal somatic cells, normal tissues, malignant tumors, and cancer cell lines.

FIG. 57 shows the expression of the GJB2 mRNA (SEQ ID NO: 165) in normal somatic cells, normal tissues, malignant tumors, and cancer cell lines.

FIG. 58 shows the relative expression of SP100 in diverse cultured normal somatic cell types including coronary artery endothelial cells (mesoderm), astrocytes (ectoderm), bronchial epithelial cells (endoderm), melanocytes (neural crest) as well as diverse clonal hES-derived embryonic progenitor cell lines compared to hES and iPS cells as measured by Illumina microarray analysis. All hES and established iPS cell lines showed no evidence of SP100 transcripts above background signal.

FIG. 59 shows the expression level of MMP11 in normal bladder tissue and cancerous bladder tissue by qPCR.

FIG. 60 shows the expression level of MMP12 in normal bladder tissue and cancerous bladder tissue by qPCR.

FIG. 61 shows the expression level of COL10A1 in normal bladder tissue and cancerous bladder tissue by qPCR.

FIG. 62 shows the expression level of FCRLBin normal bladder tissue and cancerous bladder tissue by qPCR.

FIG. 63 shows the expression level of SERPINB5 in normal bladder tissue and cancerous bladder tissue by qPCR.

FIG. 64 shows the expression level of SFN in normal bladder tissue and cancerous bladder tissue by qPCR.

FIG. 65 shows the expression level of KRT6Ain normal bladder tissue and cancerous bladder tissue by qPCR.

FIG. 66 shows the expression level of FCRLB in normal bladder tissue and cancerous bladder tissue by qPCR.

FIG. 67 shows the expression level of IL1A in normal bladder tissue and cancerous bladder tissue by qPCR.

FIG. 68 shows the expression level of KRT16 in normal bladder tissue and cancerous bladder tissue by qPCR.

FIG. 69 shows the expression level of SLC1A6 in normal bladder tissue and cancerous bladder tissue by qPCR.

FIG. 70 shows the expression level of S100A2 in normal bladder tissue and cancerous bladder tissue by qPCR.

FIG. 71 shows the expression level of S100A7A in normal bladder tissue and cancerous bladder tissue by qPCR.

FIG. 72 shows the expression level of MMP12 in normal bladder tissue and cancerous bladder tissue by ELISA.

FIG. 73 shows the expression level of ColX in normal bladder tissue and cancerous bladder tissue by ELISA.

FIG. 74 shows the expression level of MMP11 in normal bladder tissue and cancerous bladder tissue by ELISA.

FIG. 75 is agarose gel analysis of a qPCR expression data for markers COL10A1, MMP11, SFN, FCRLB in human urine.

FIG. 76 shows the expression level of SERPINB5 in normal bladder tissue and cancerous bladder tissue by qPCR.

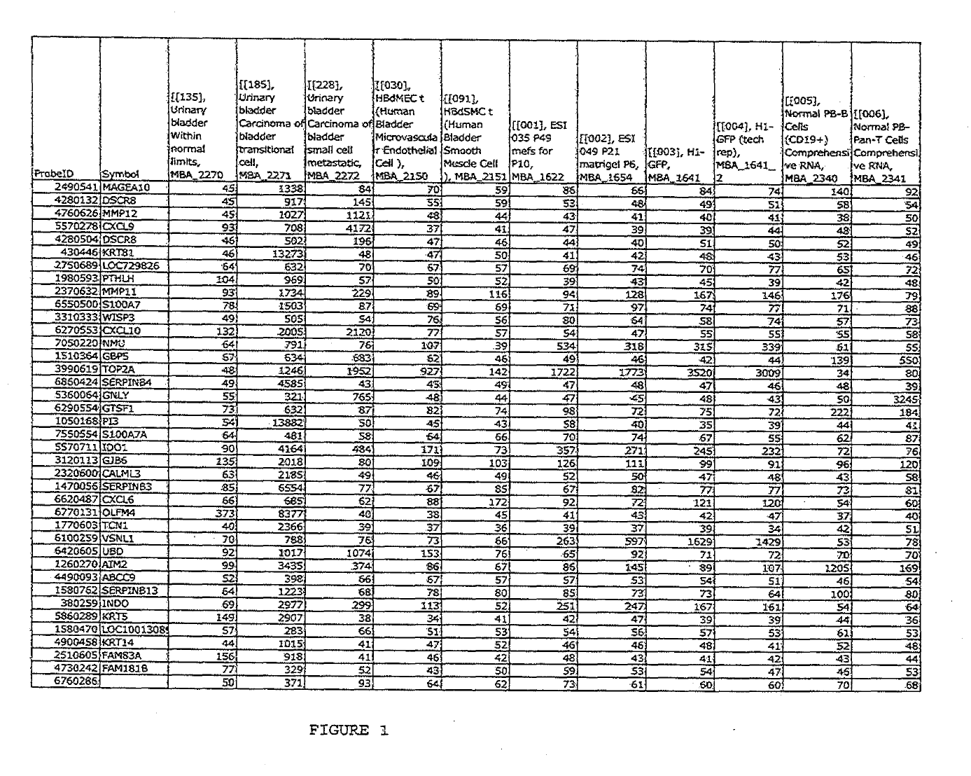

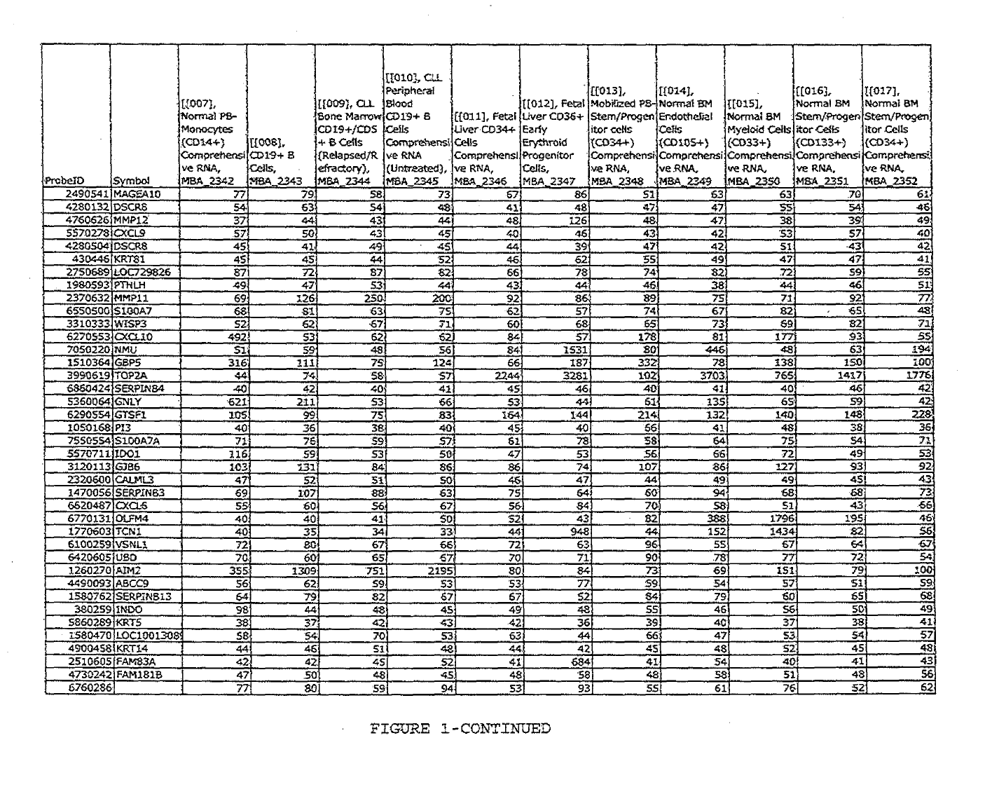

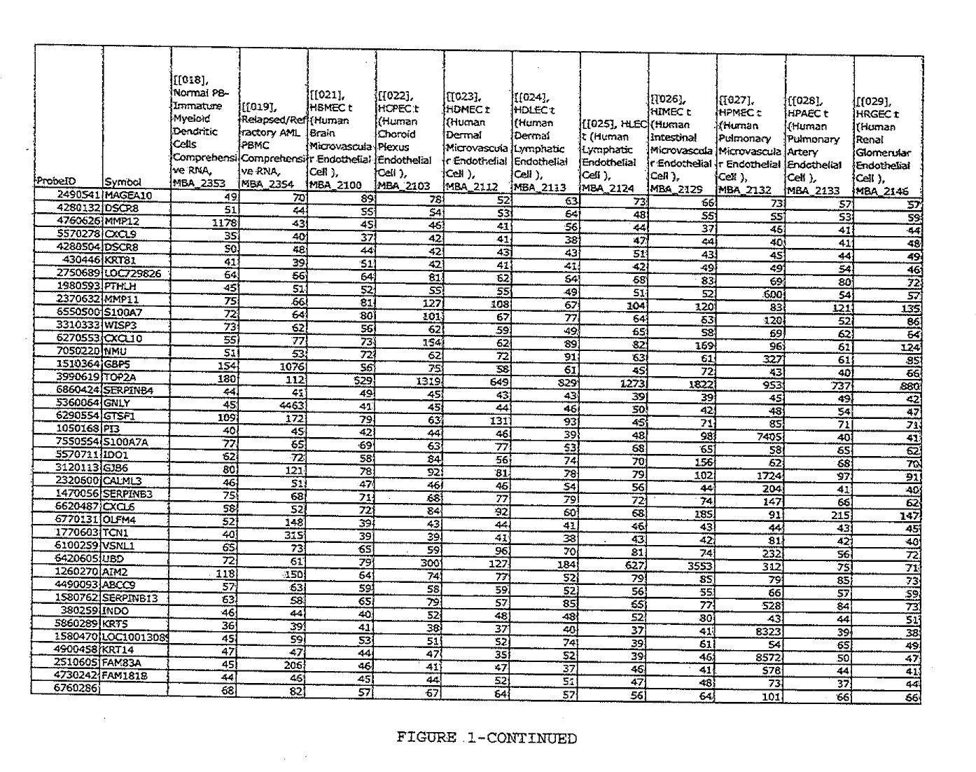

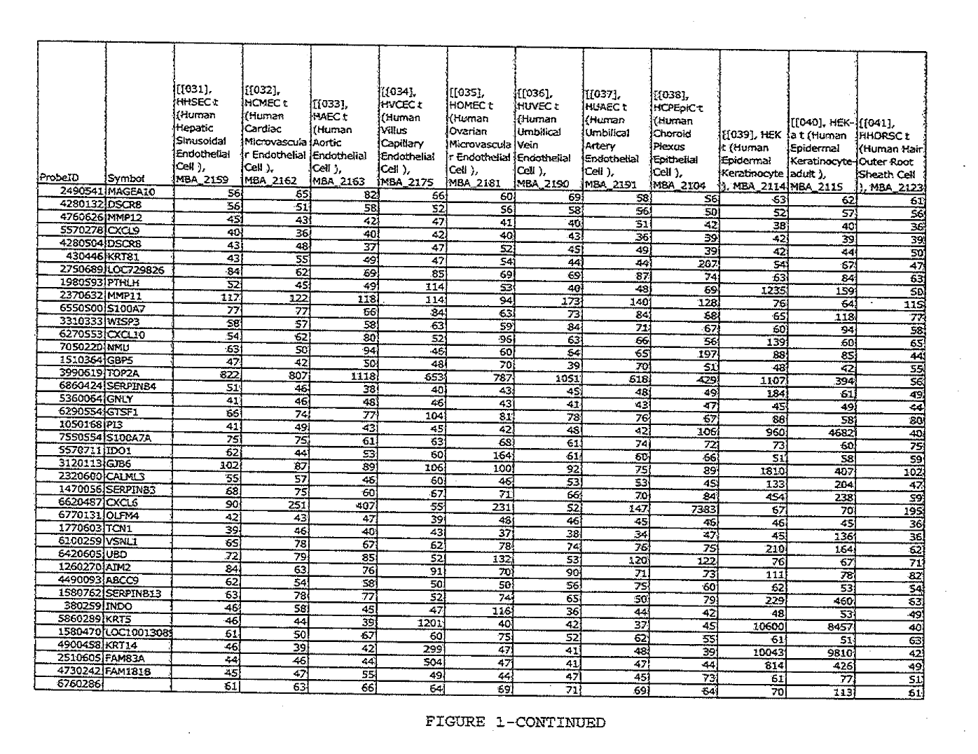

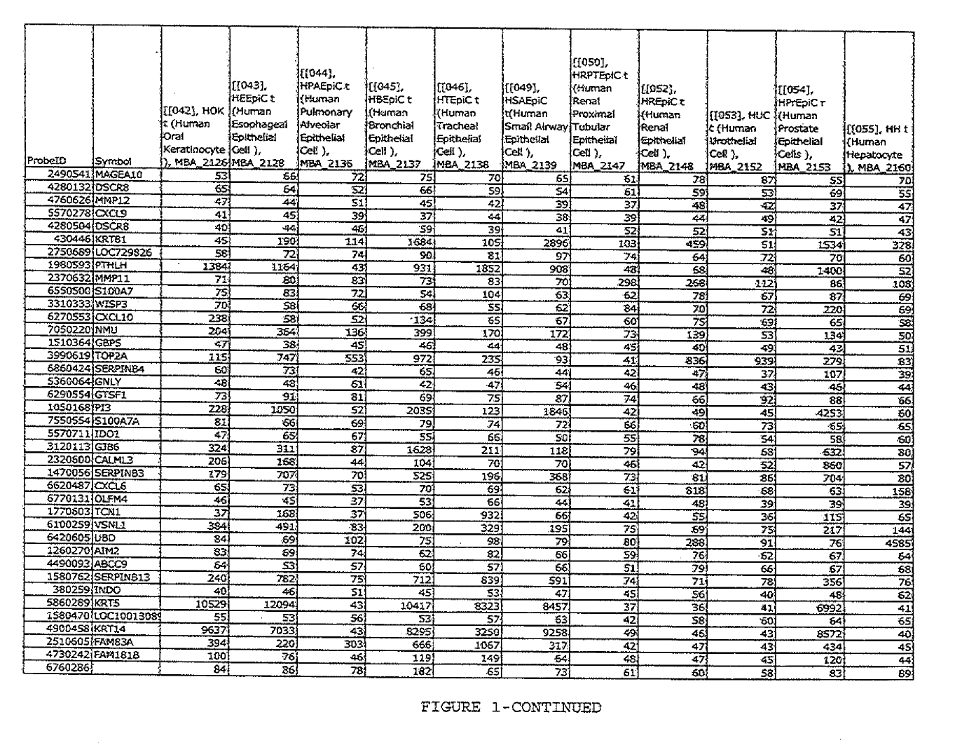

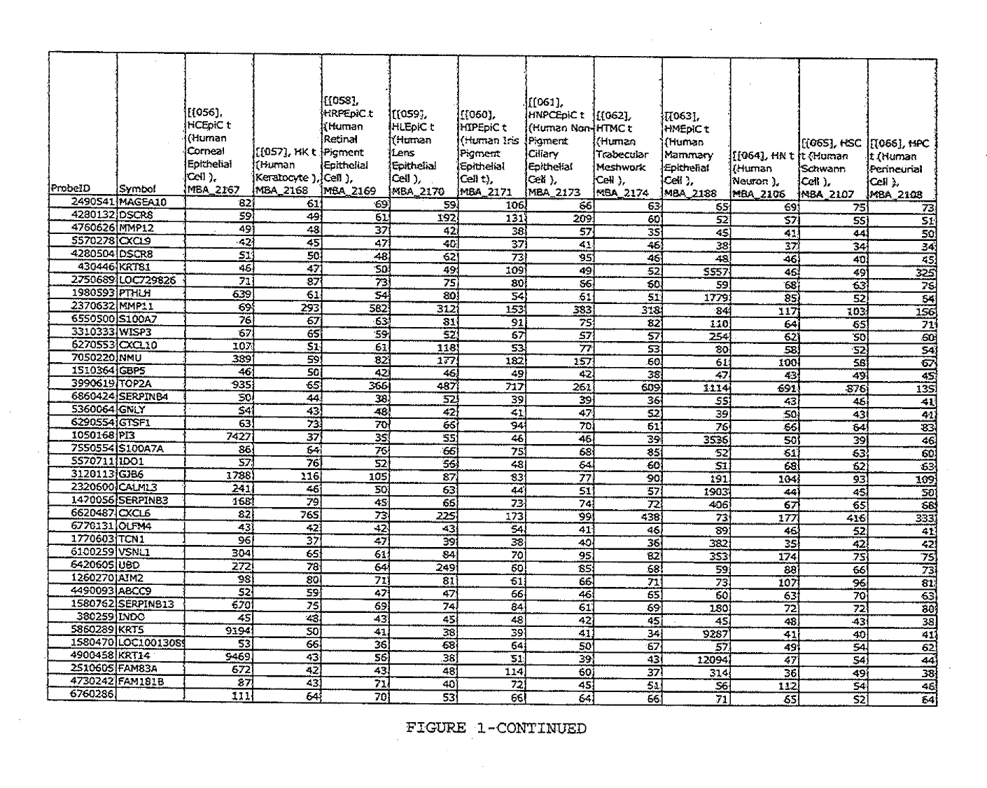

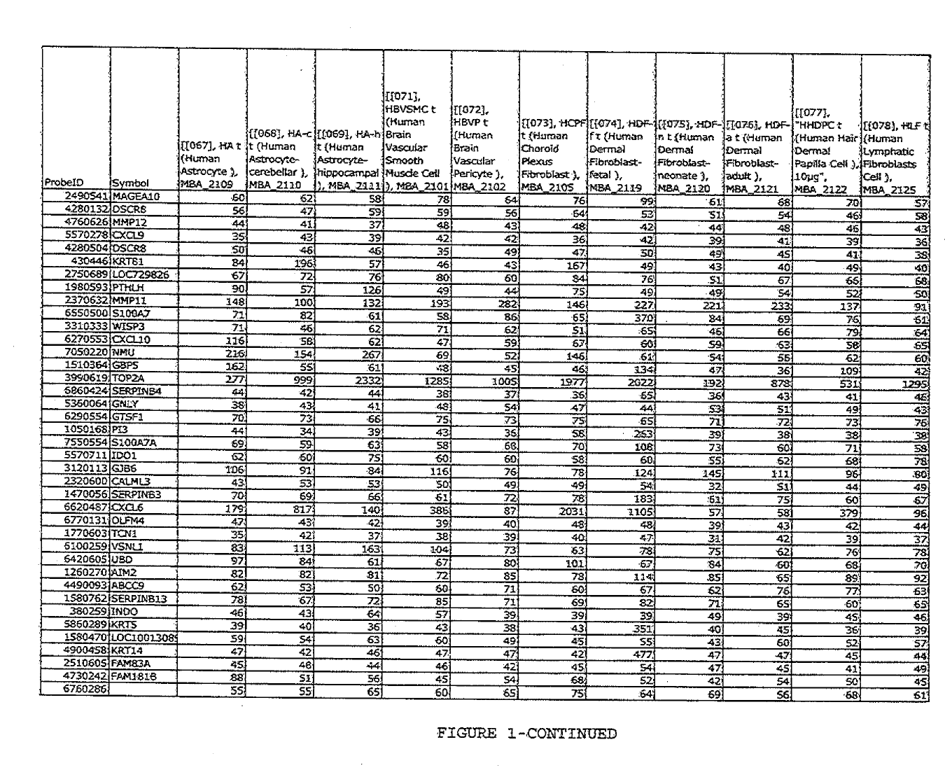

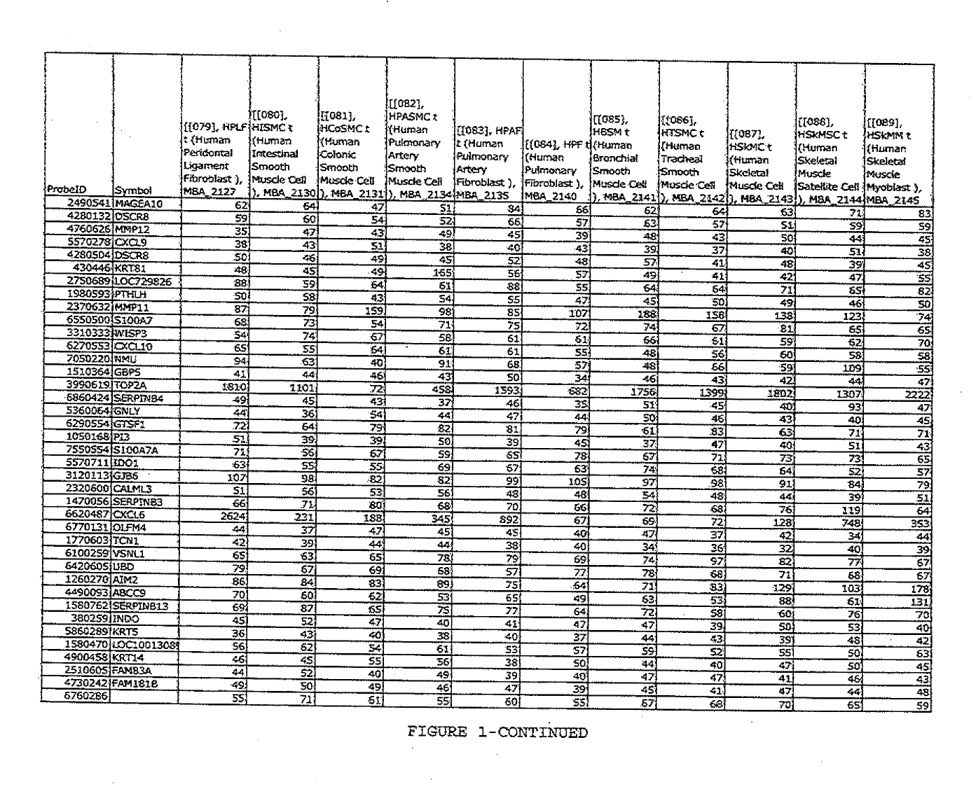

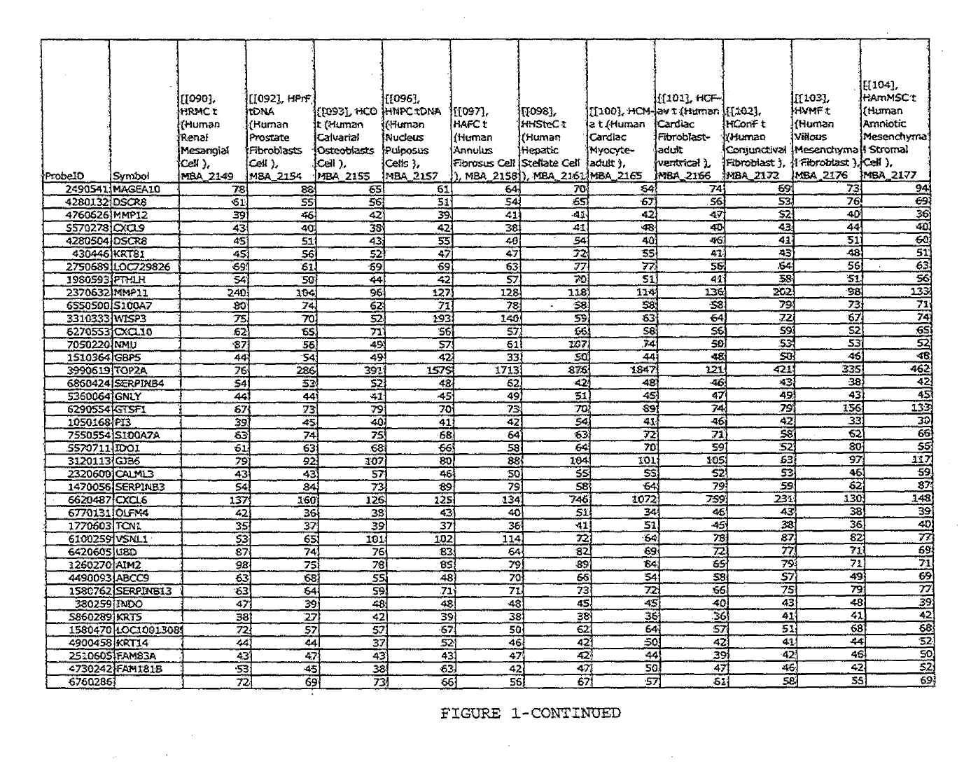

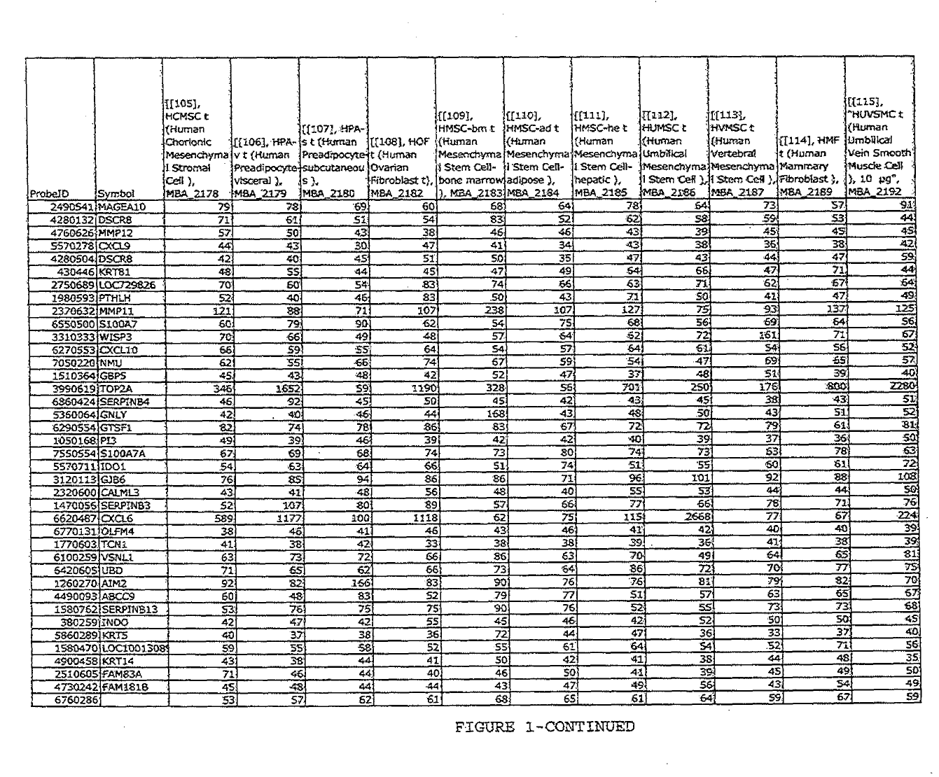

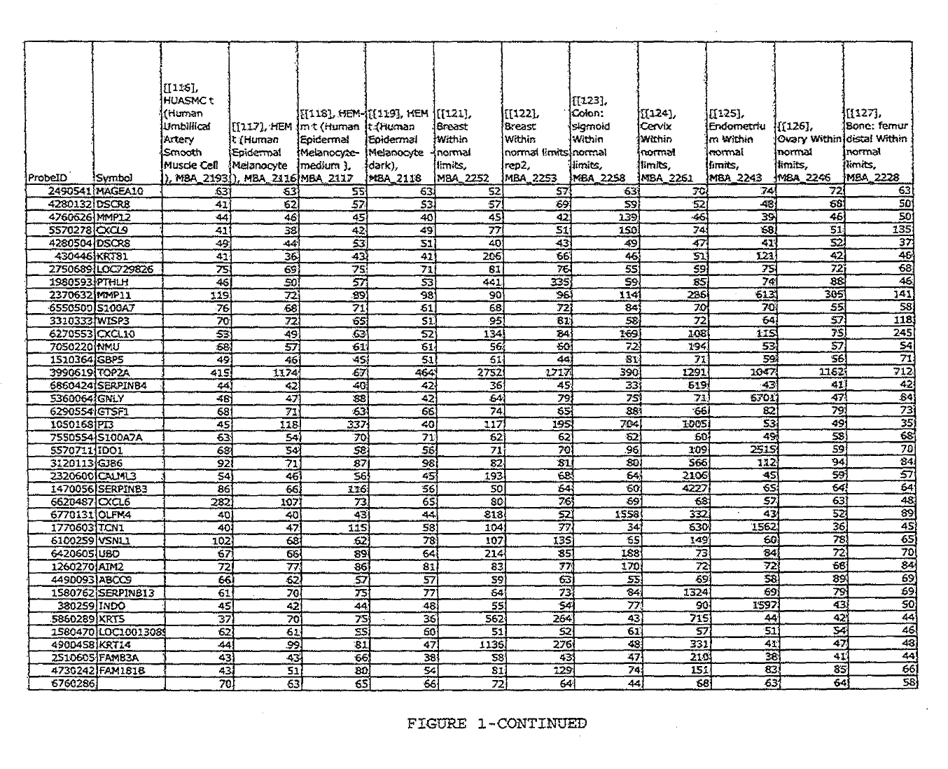

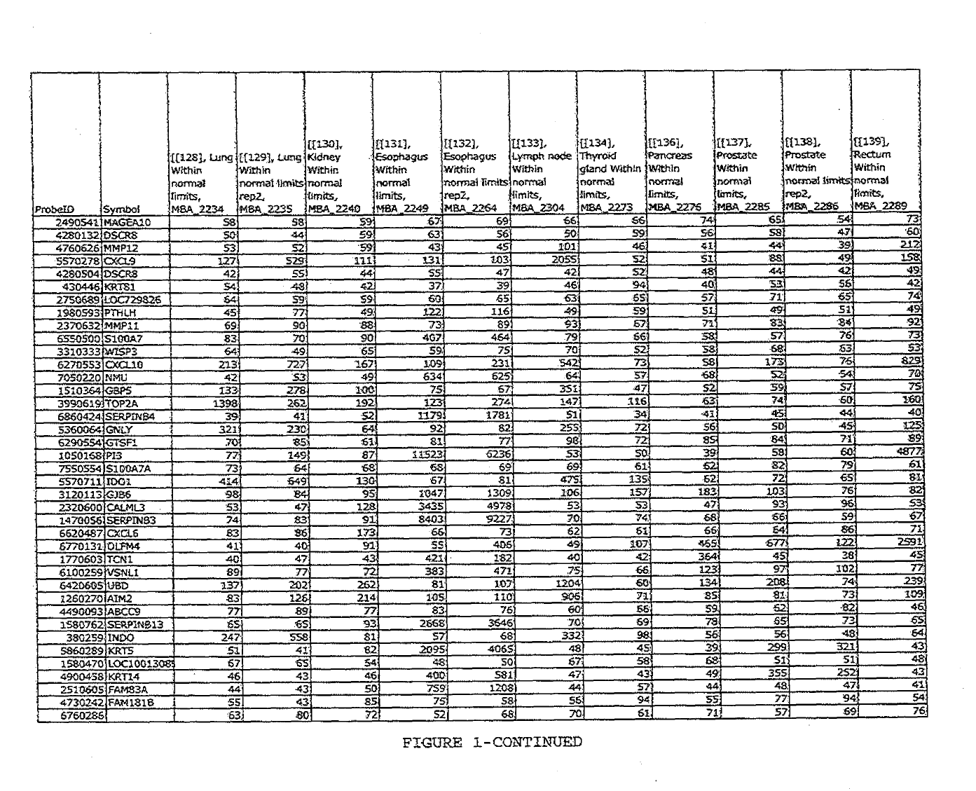

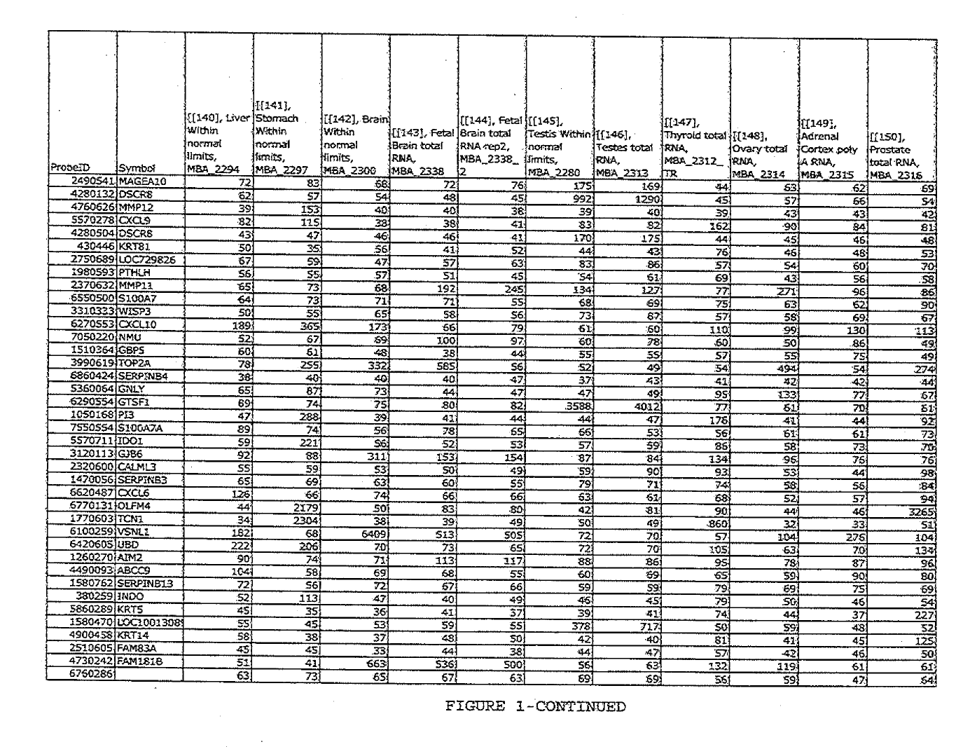

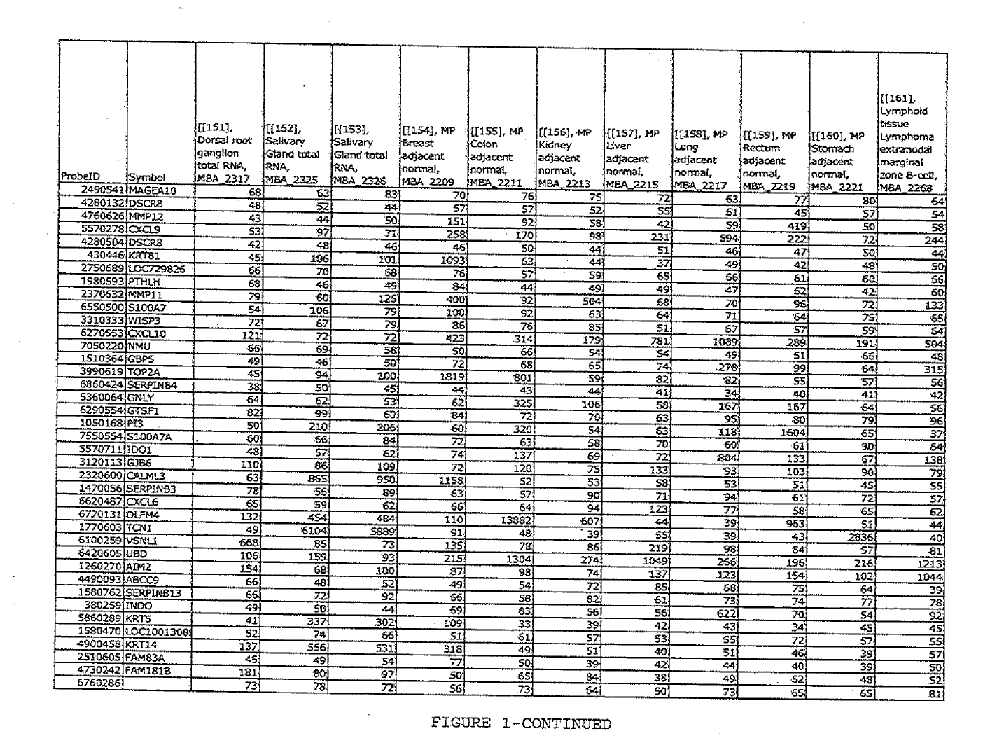

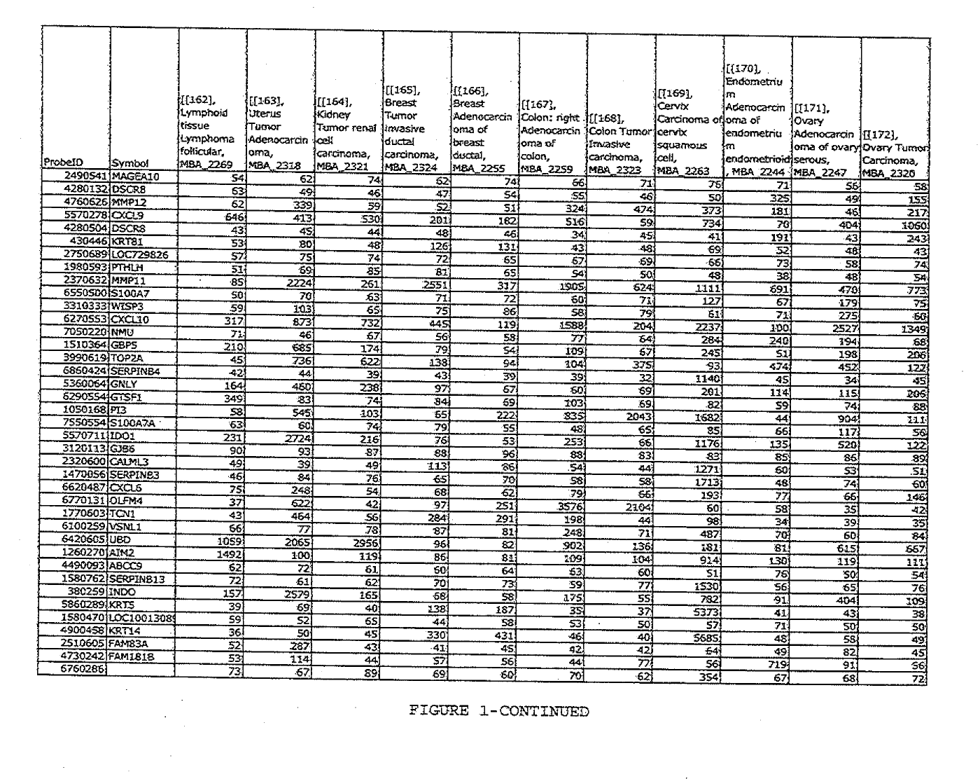

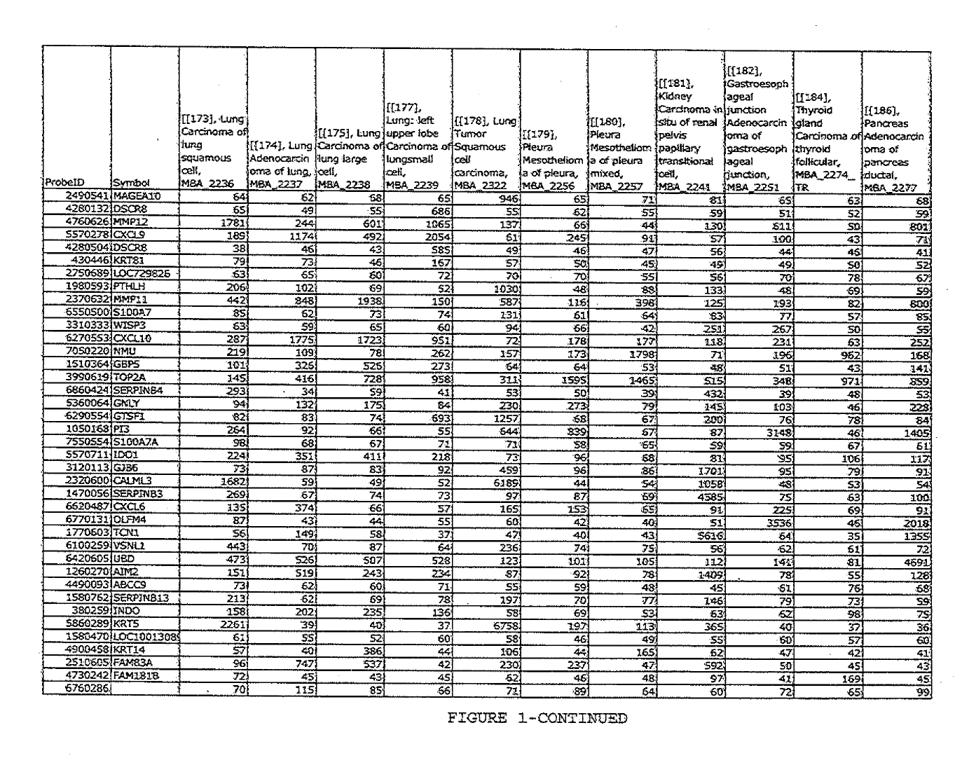

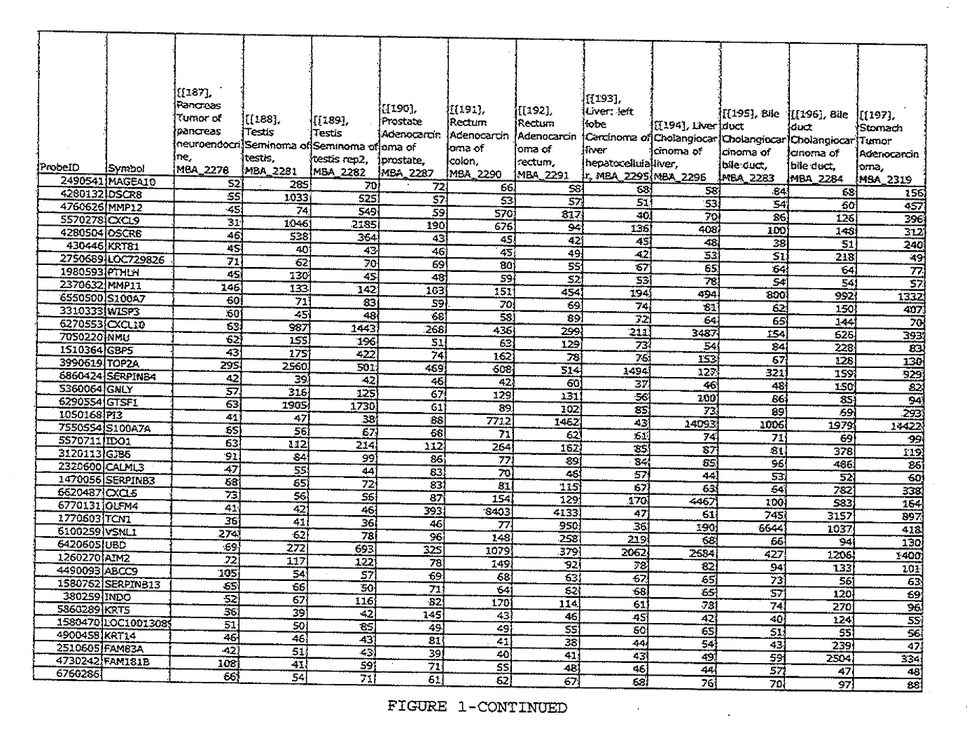

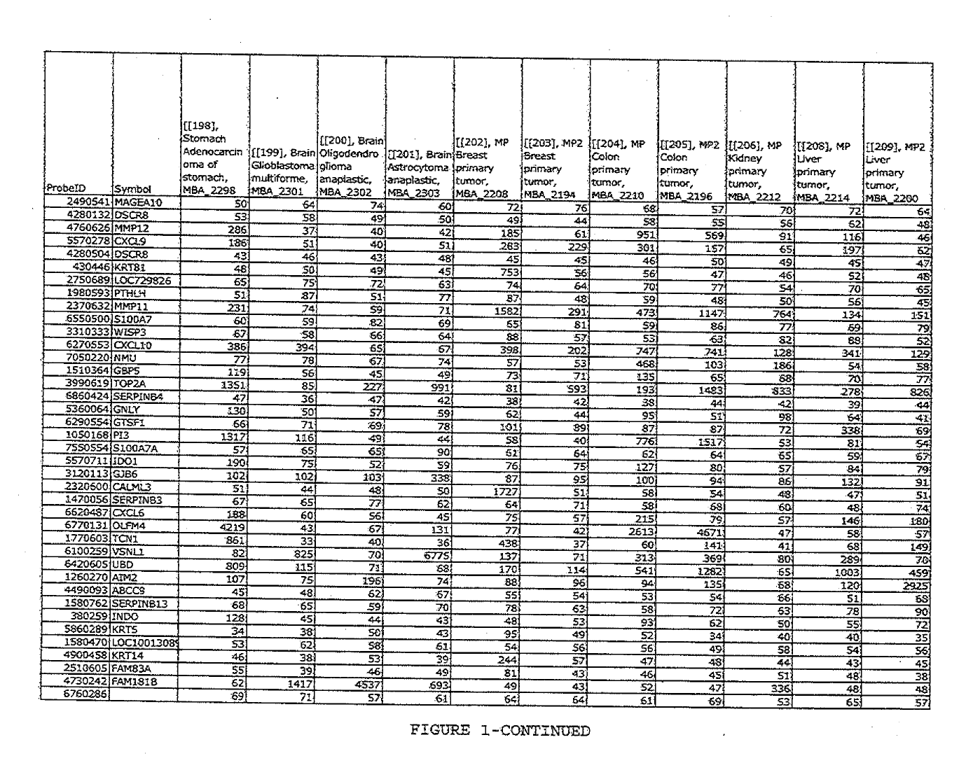

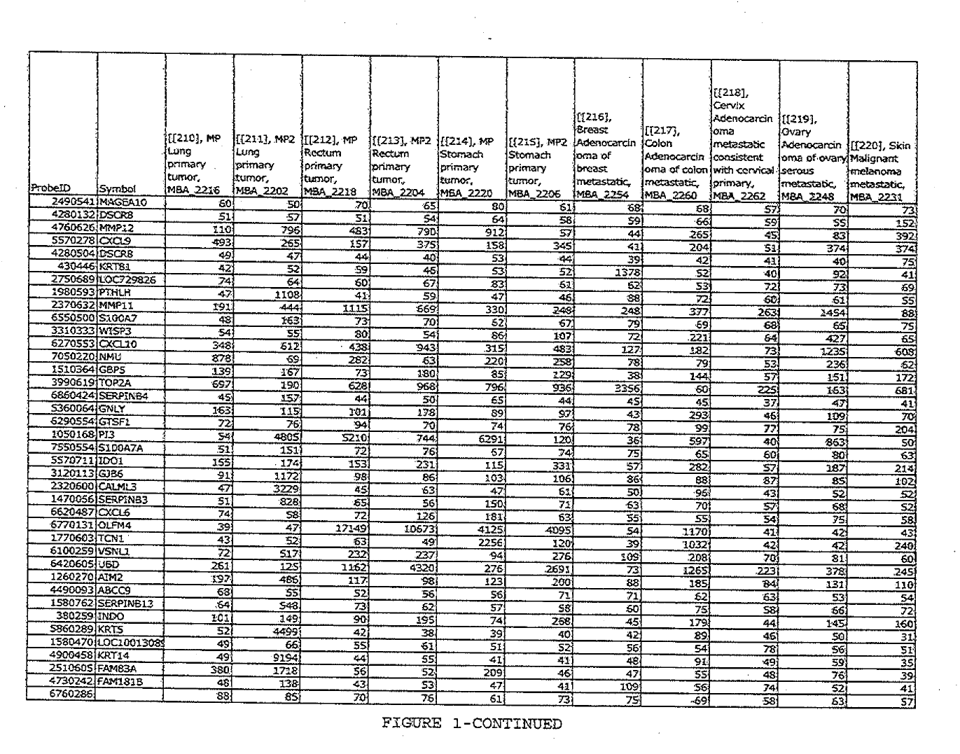

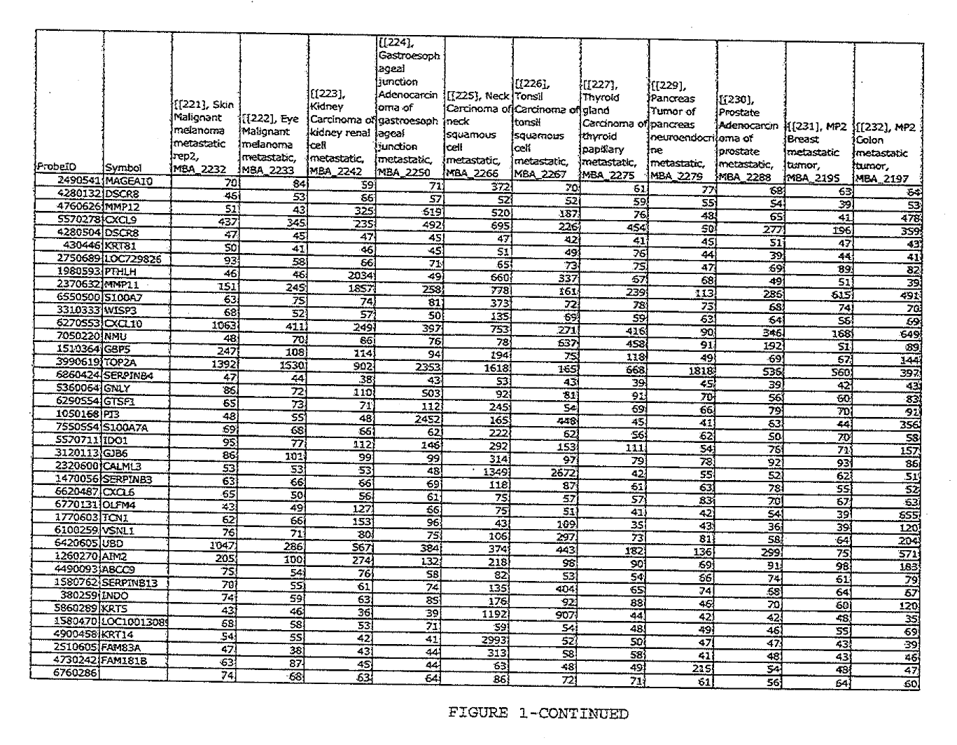

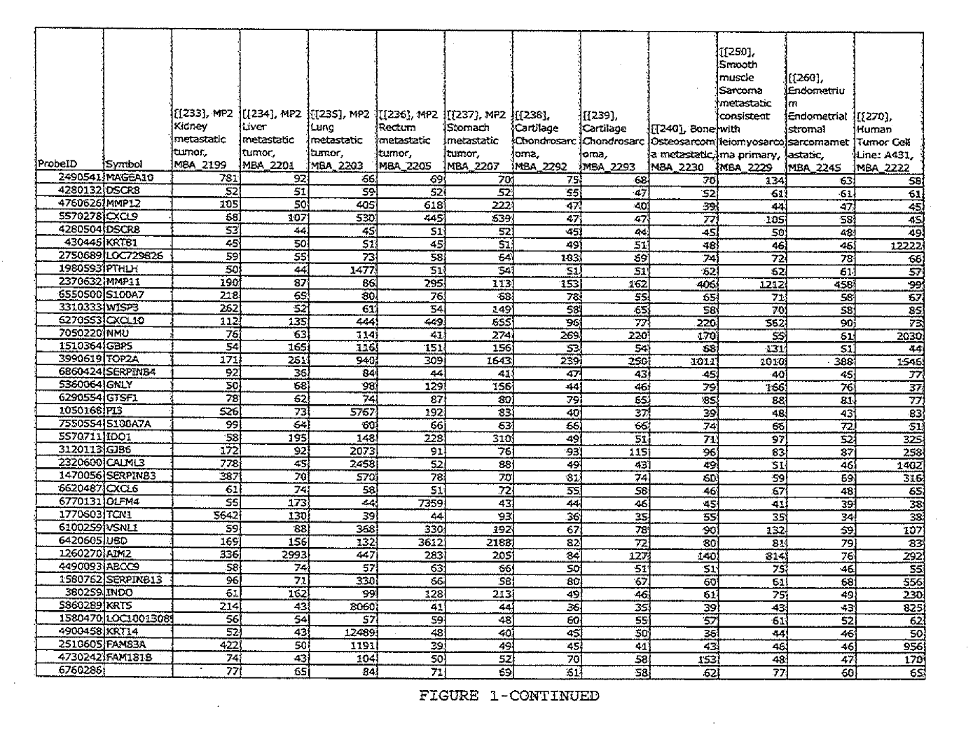

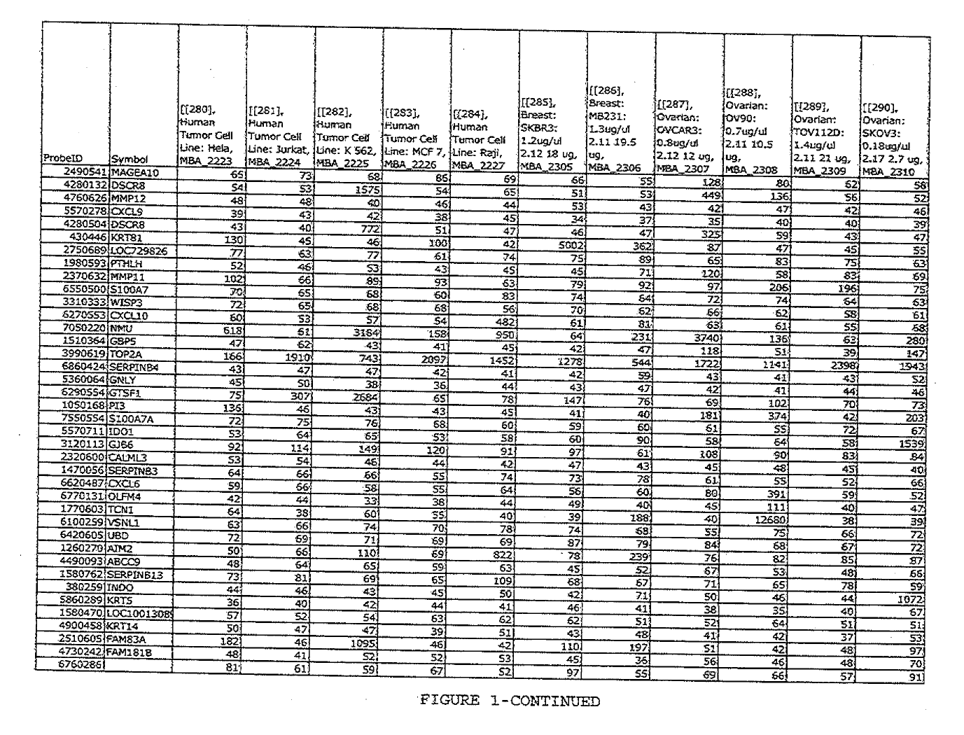

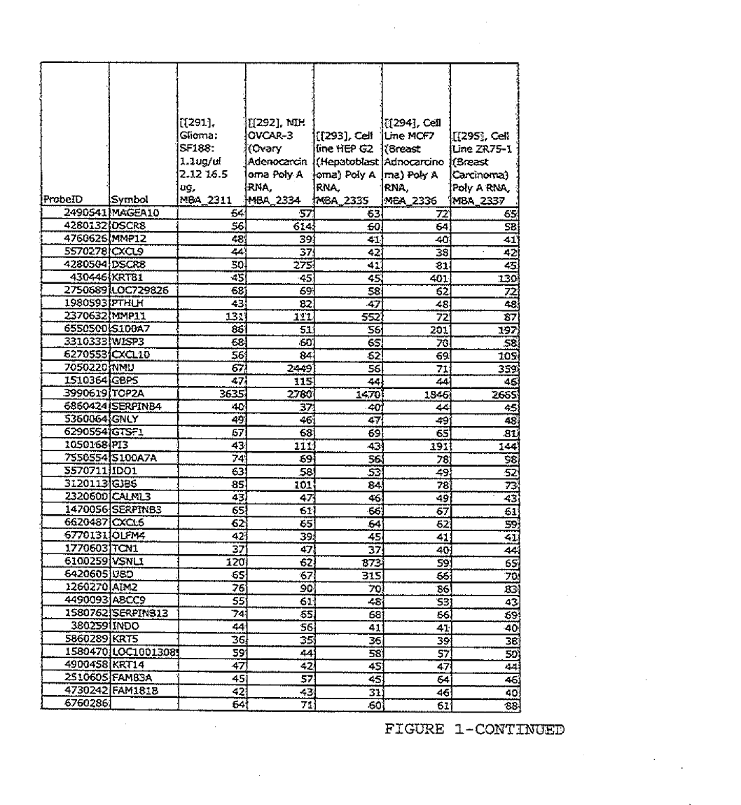

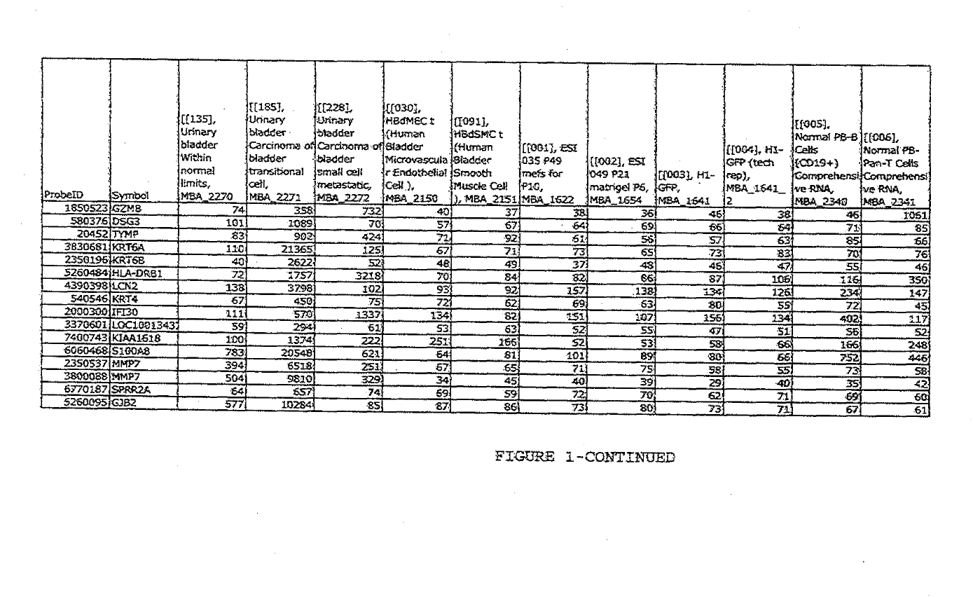

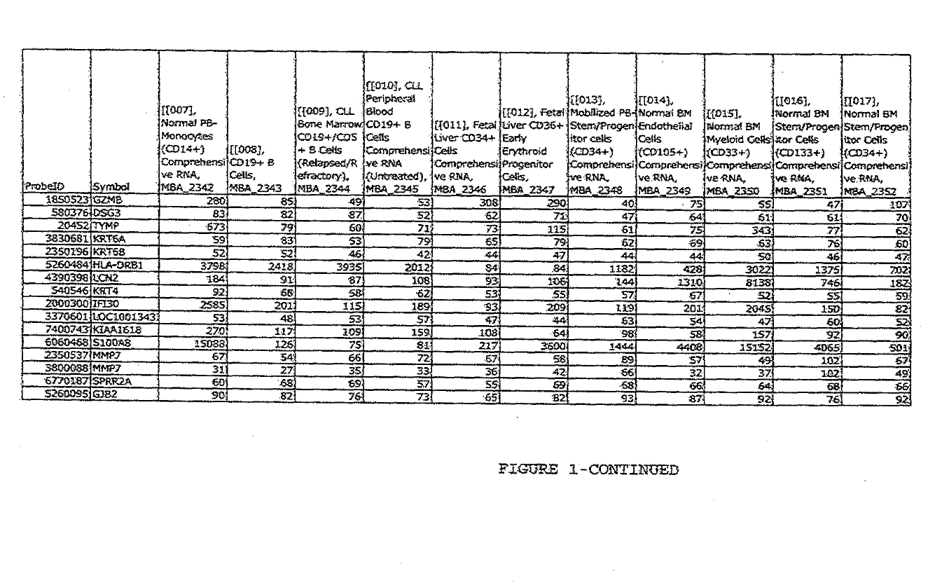

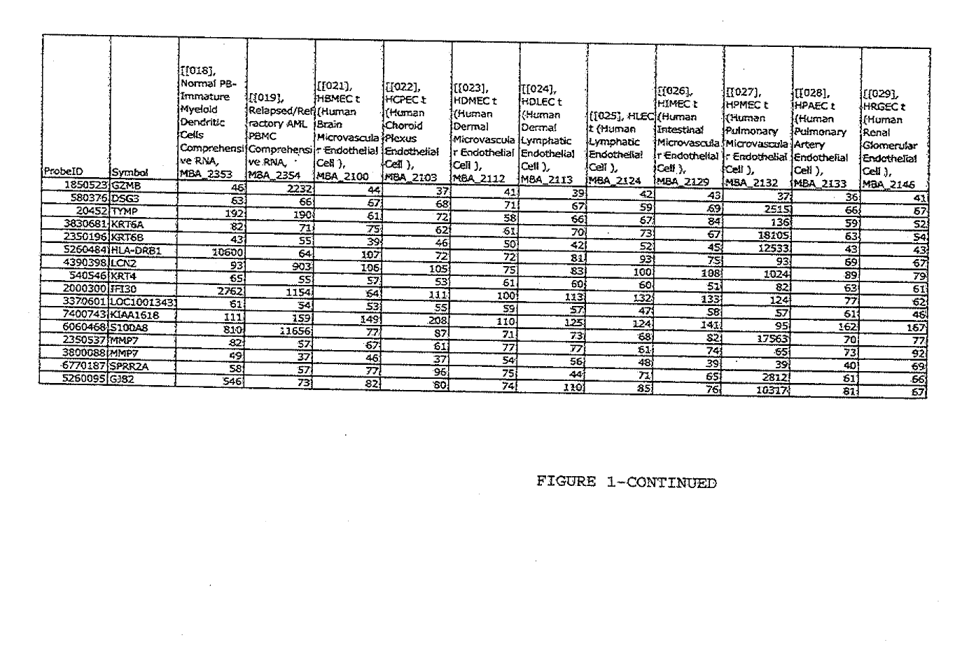

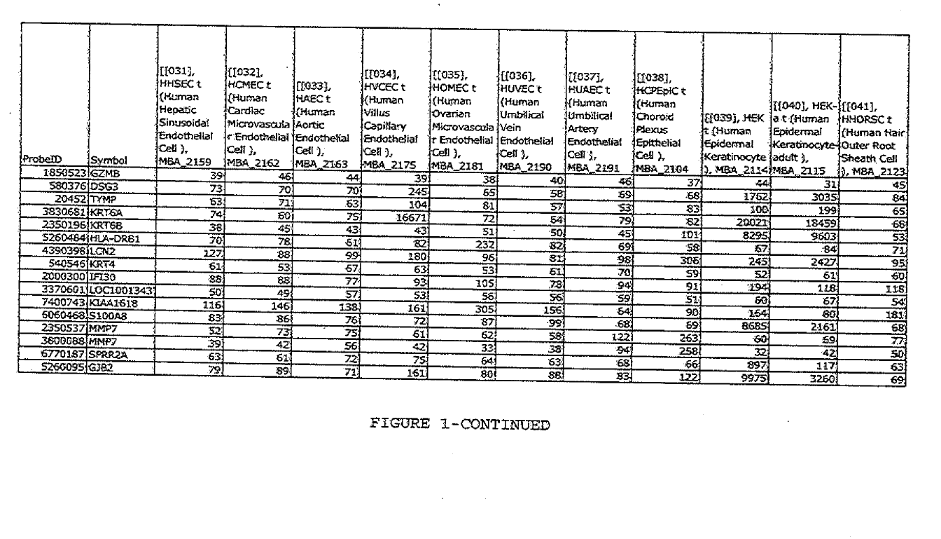

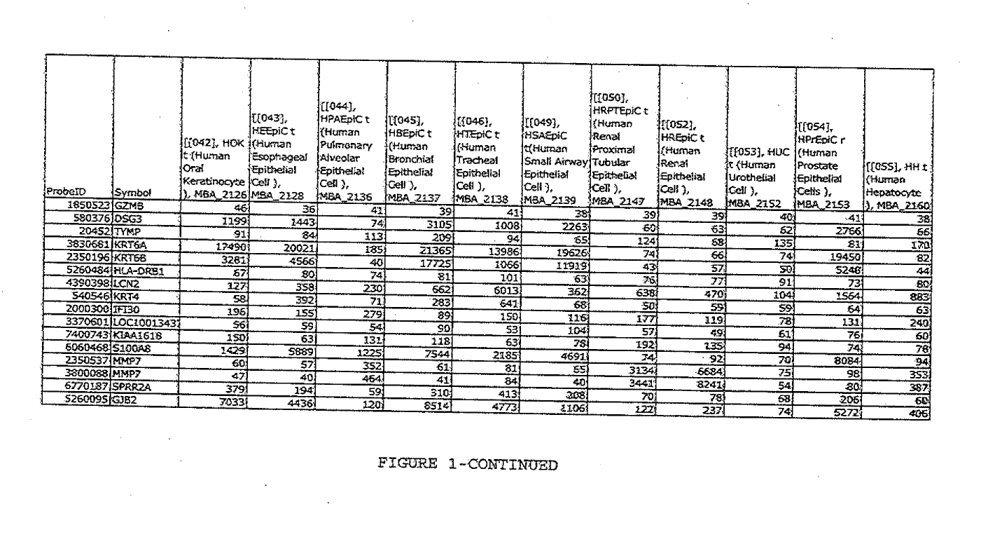

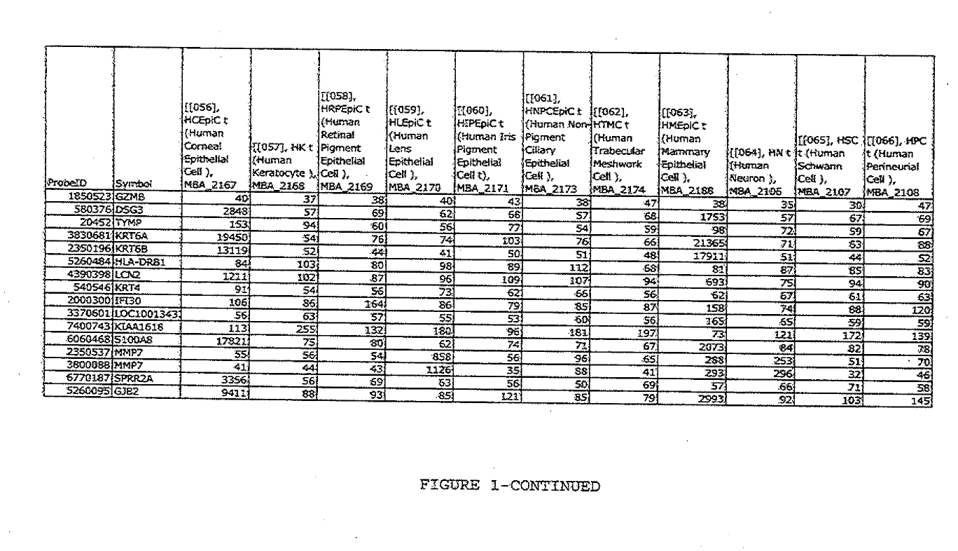

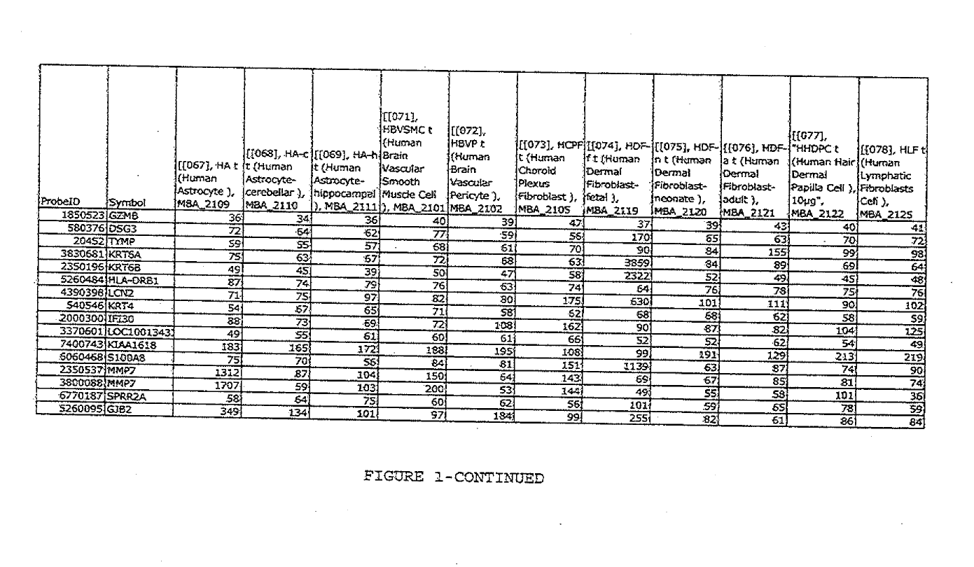

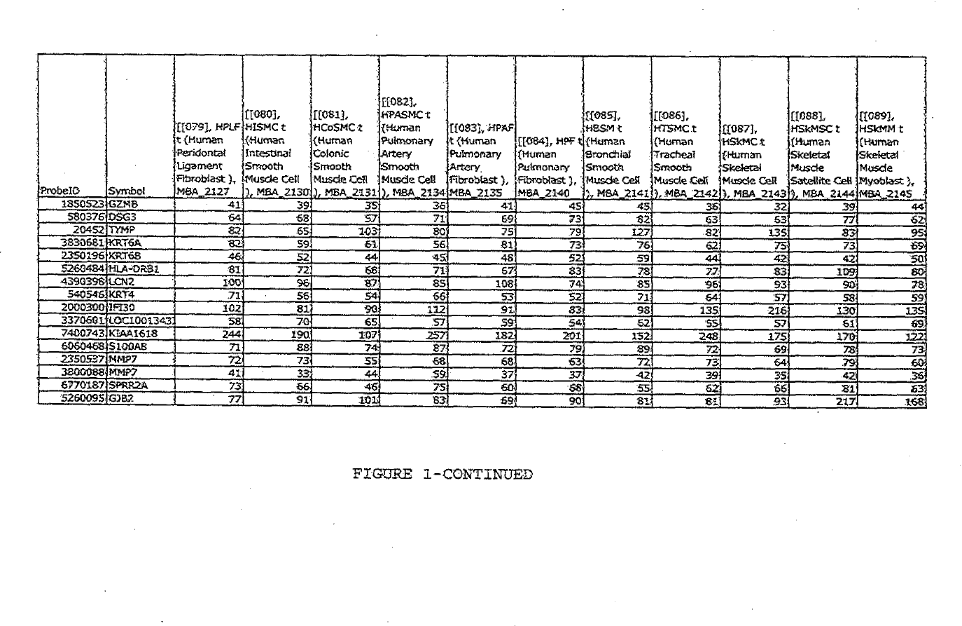

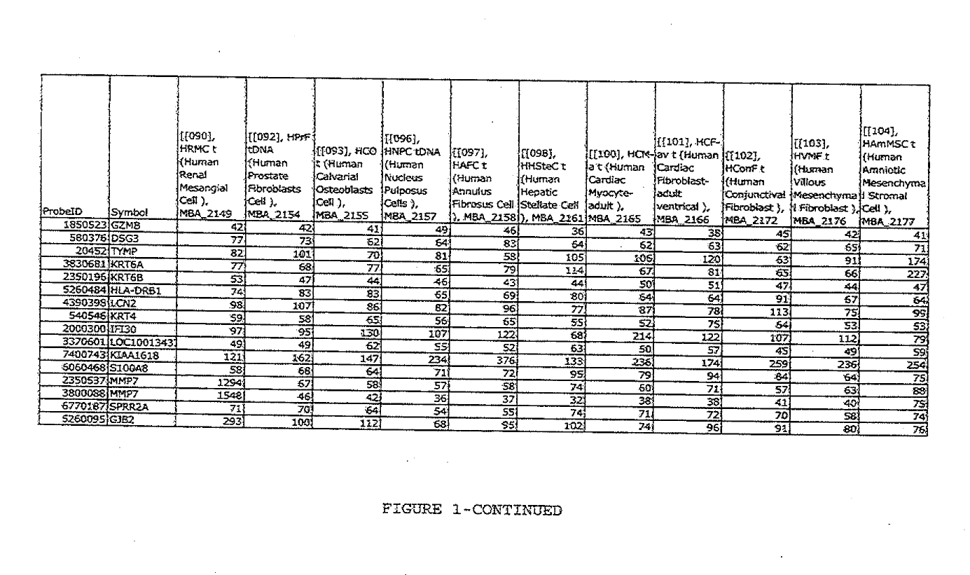

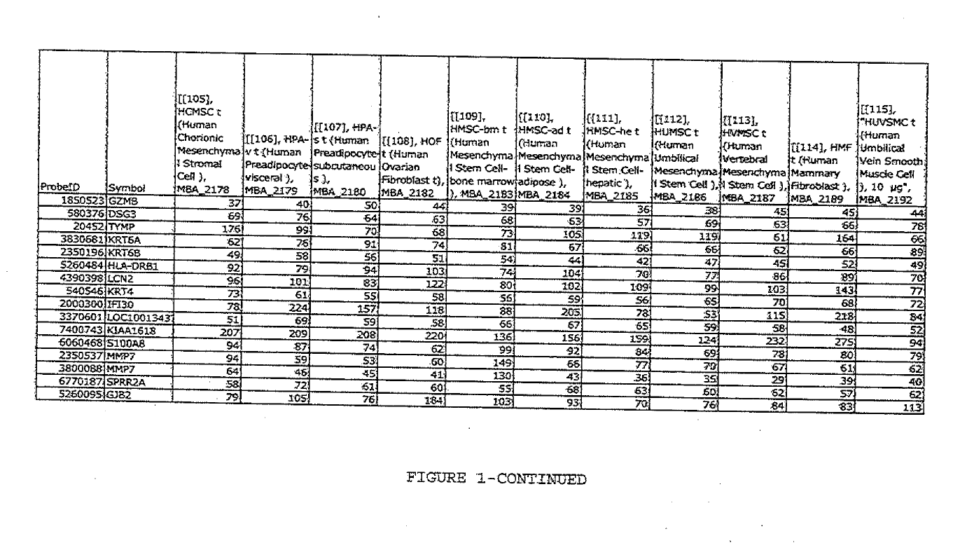

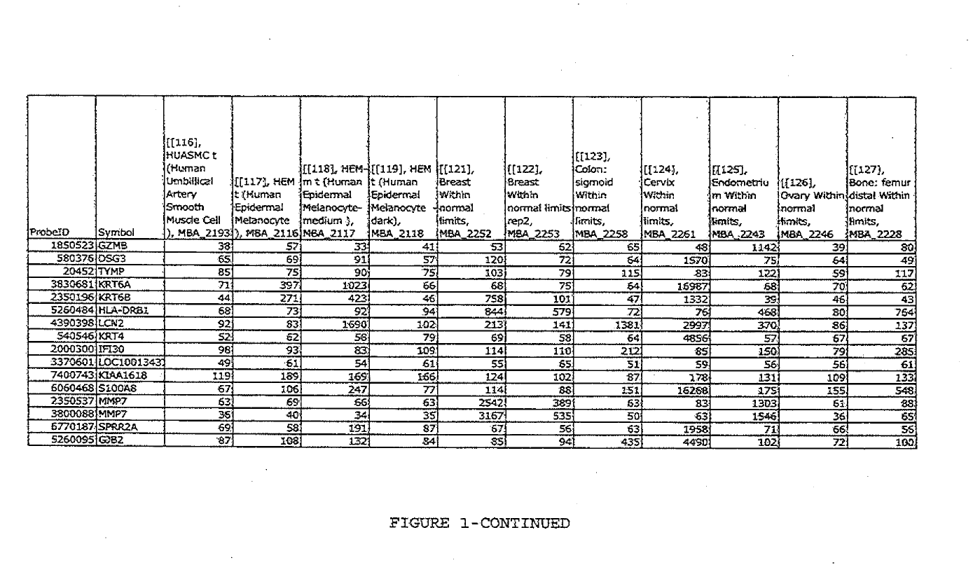

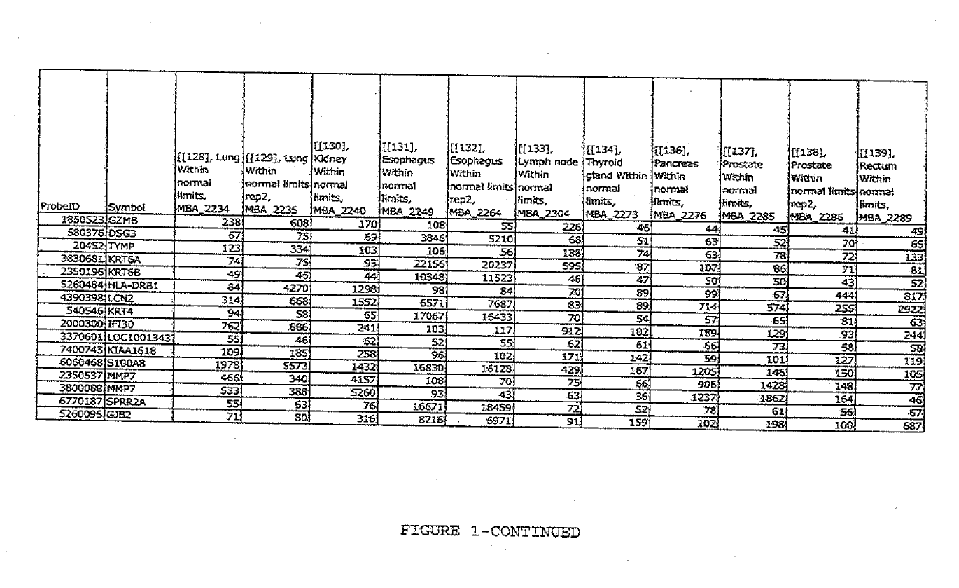

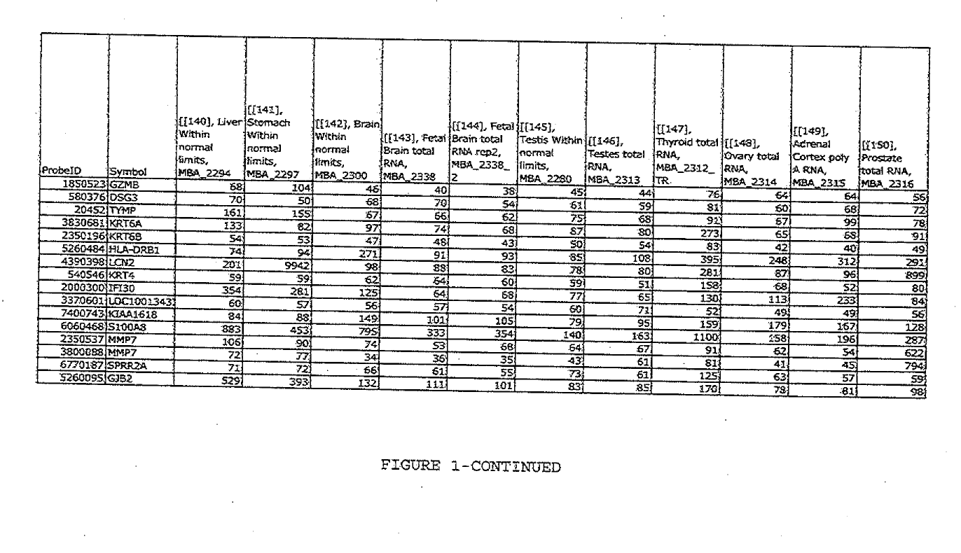

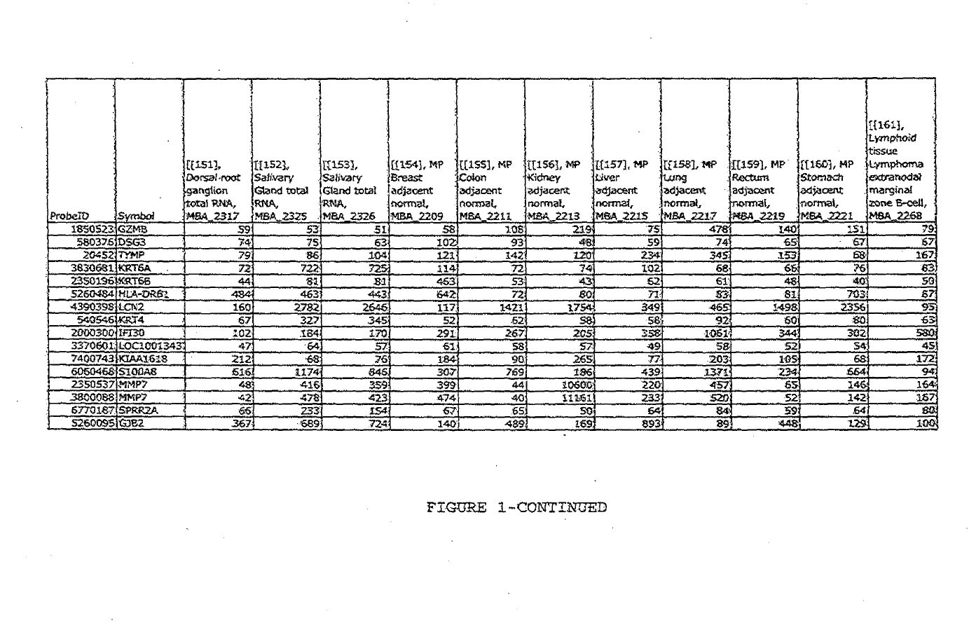

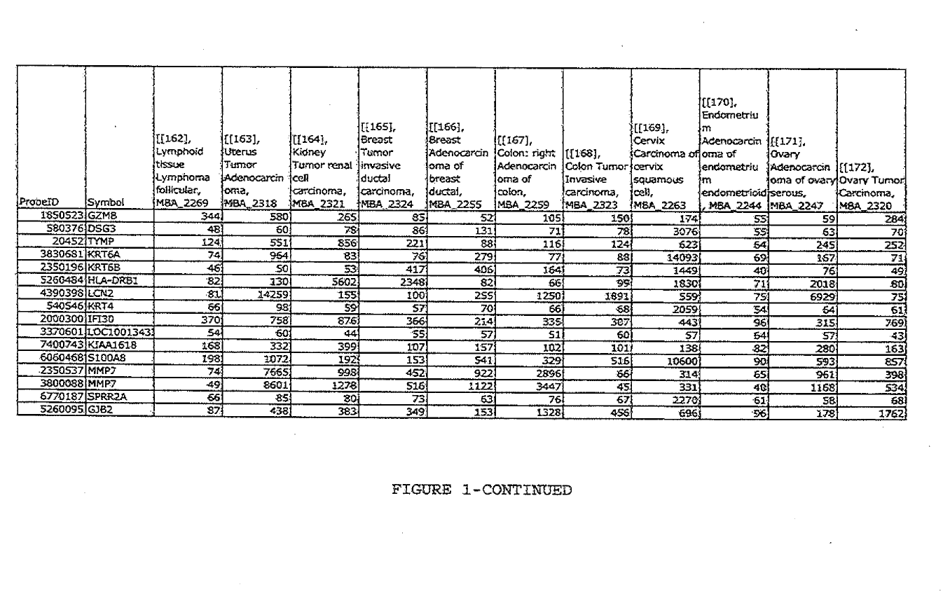

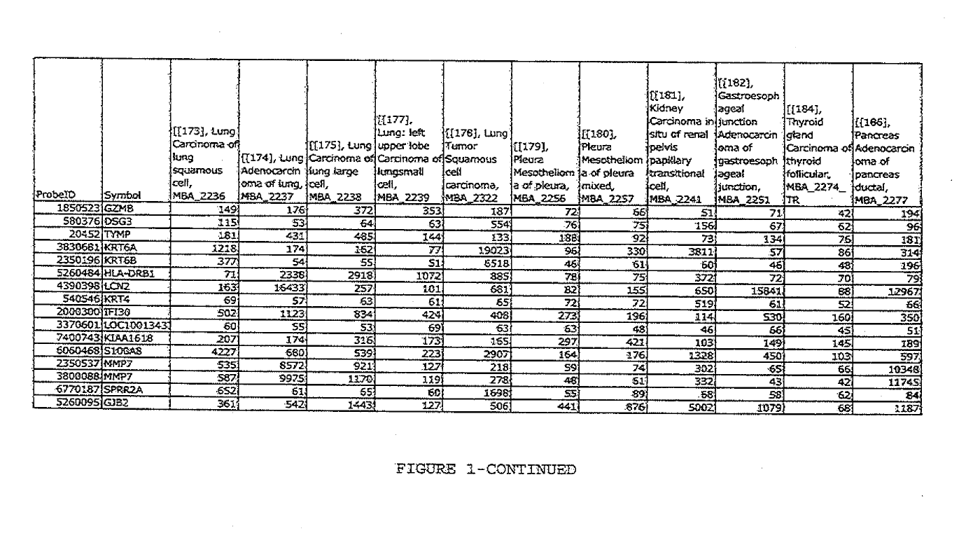

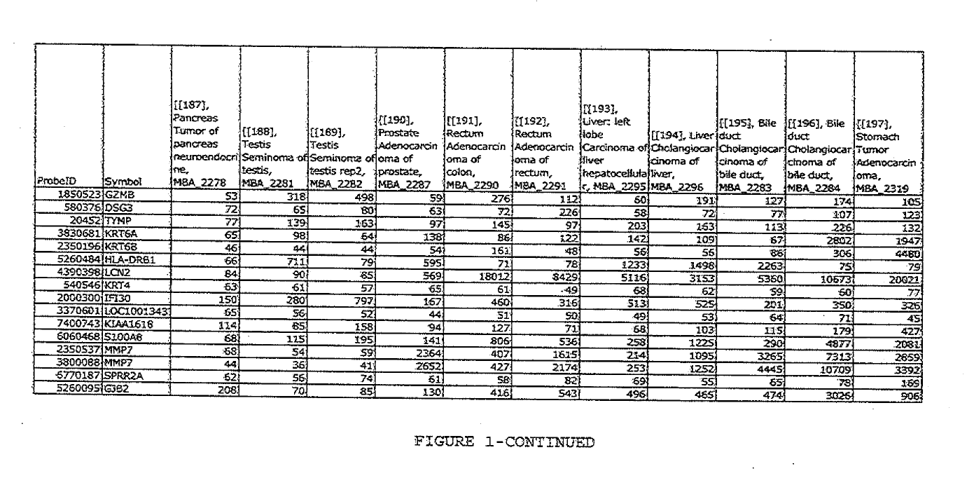

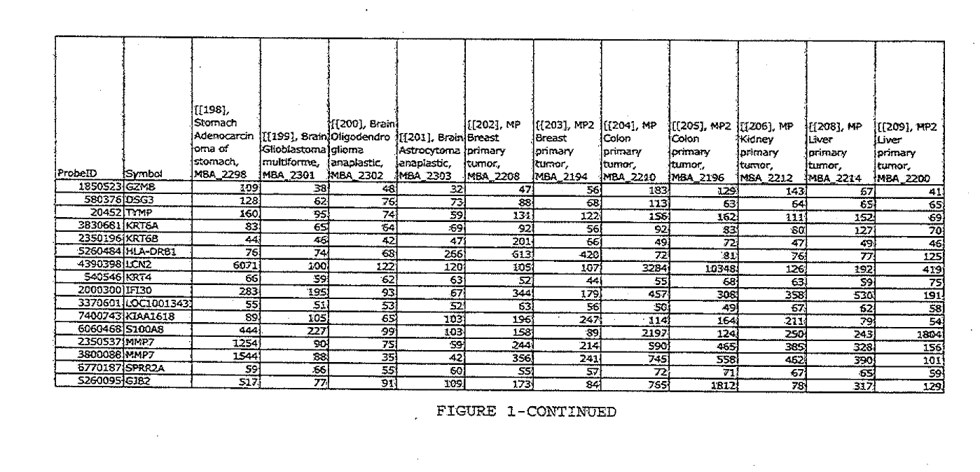

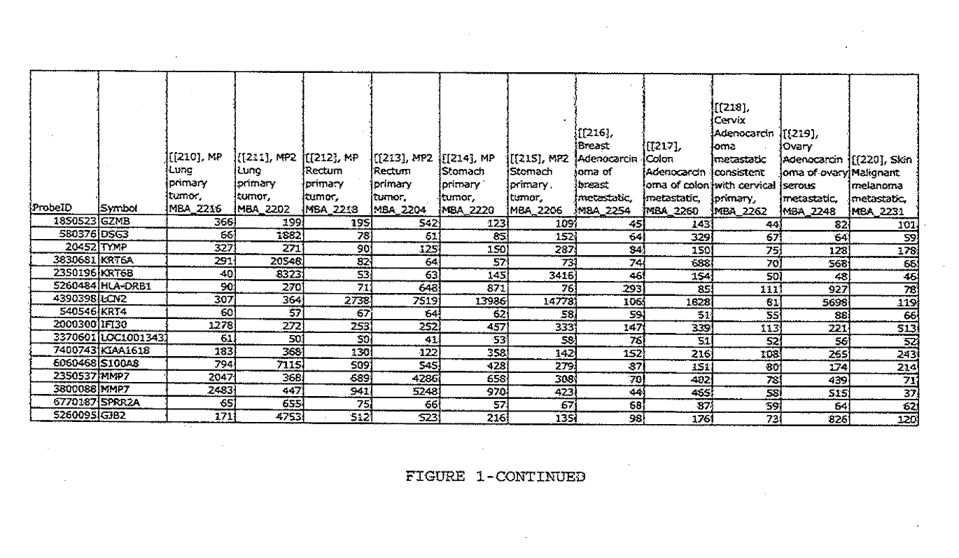

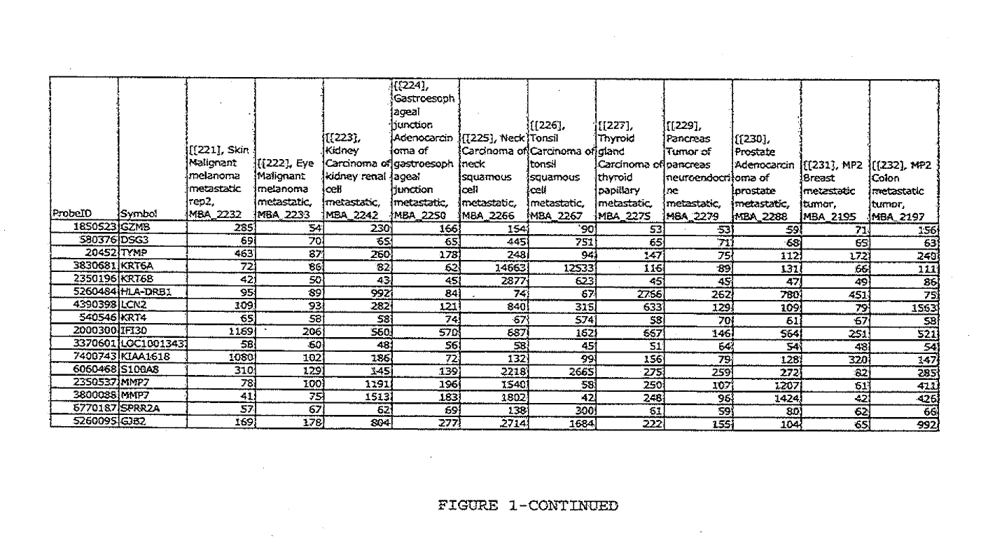

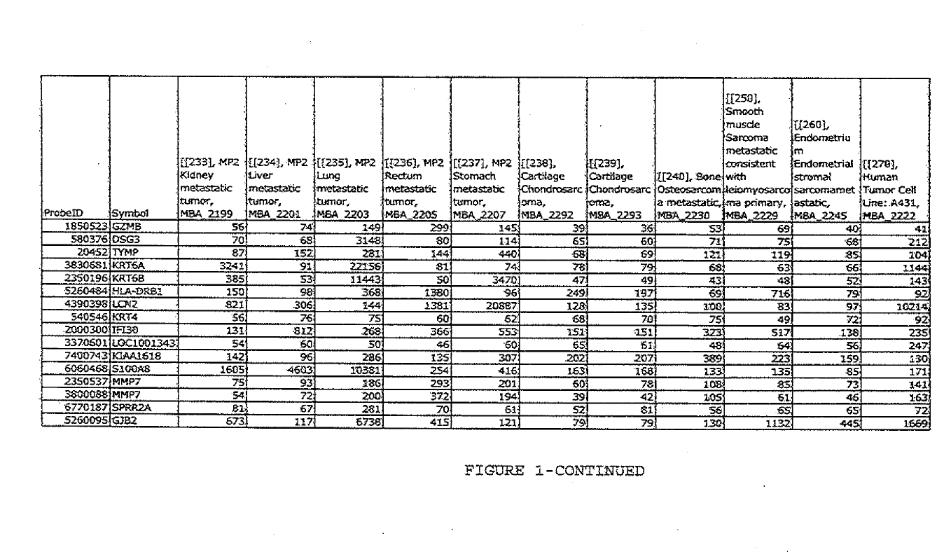

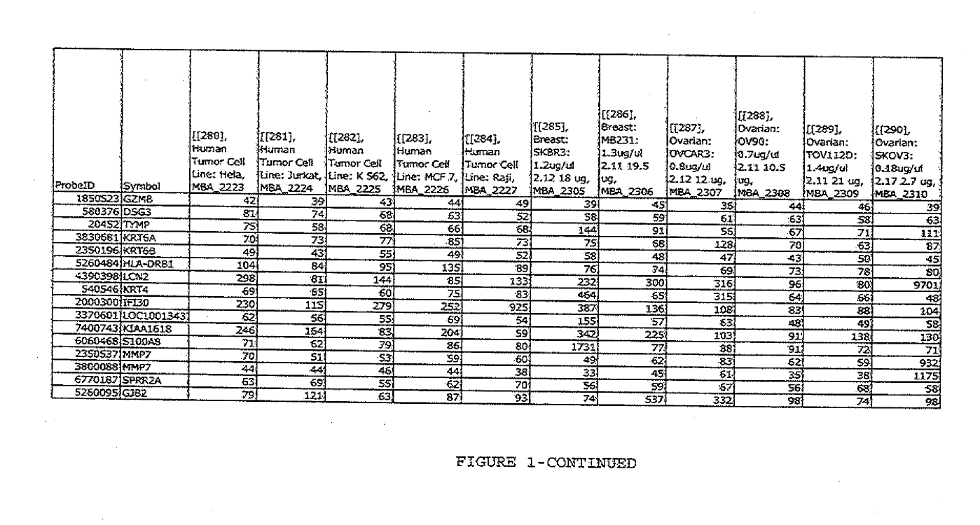

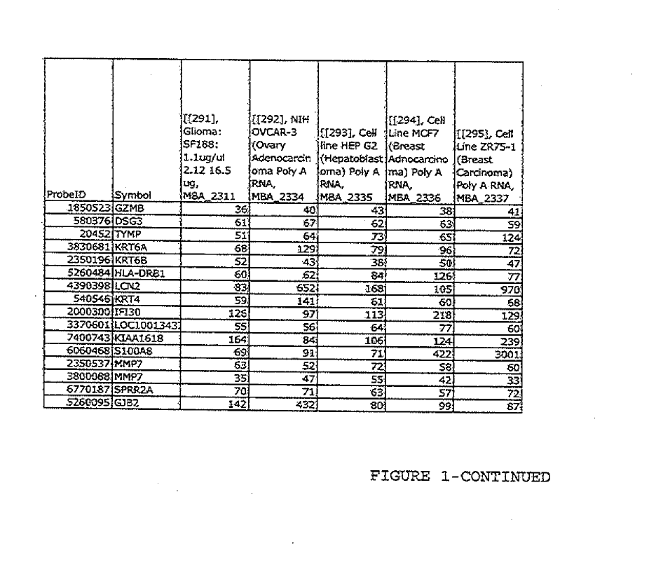

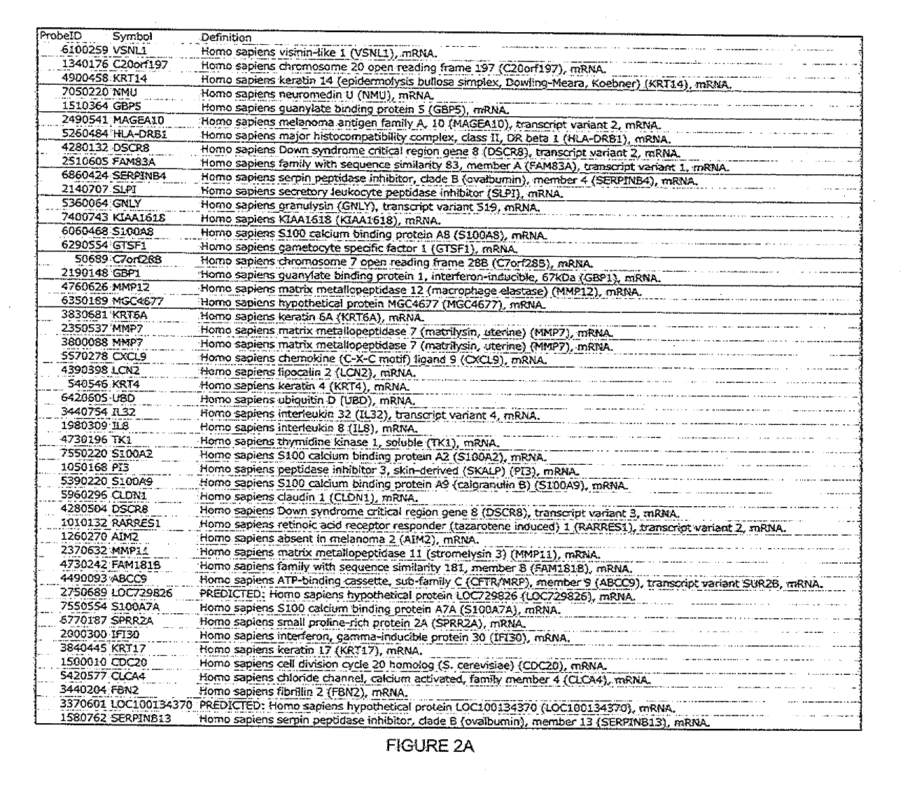

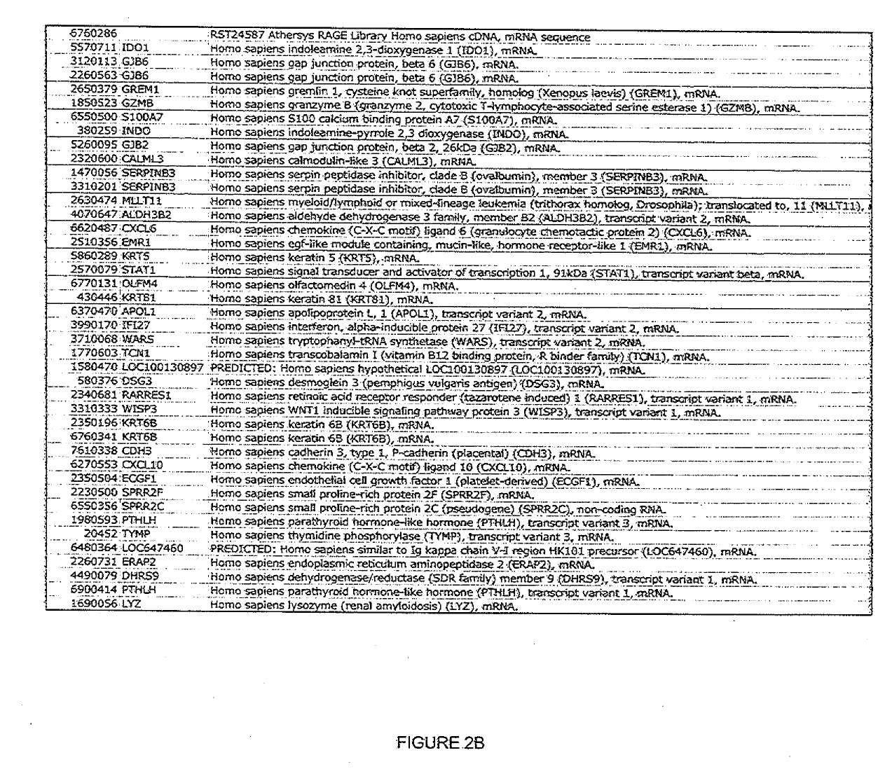

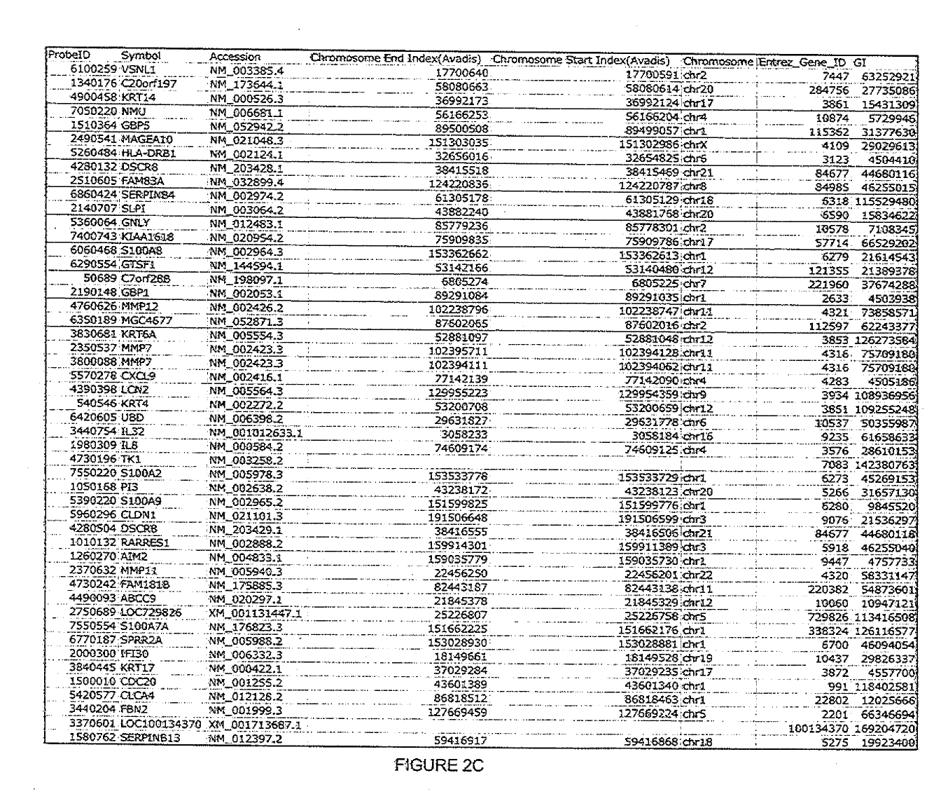

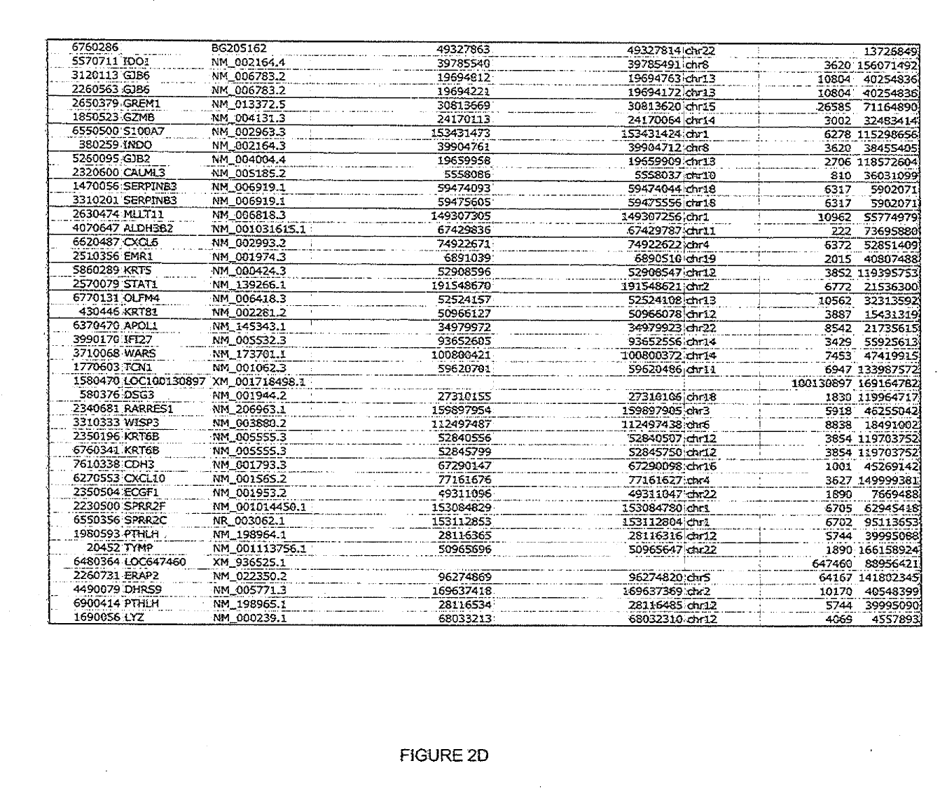

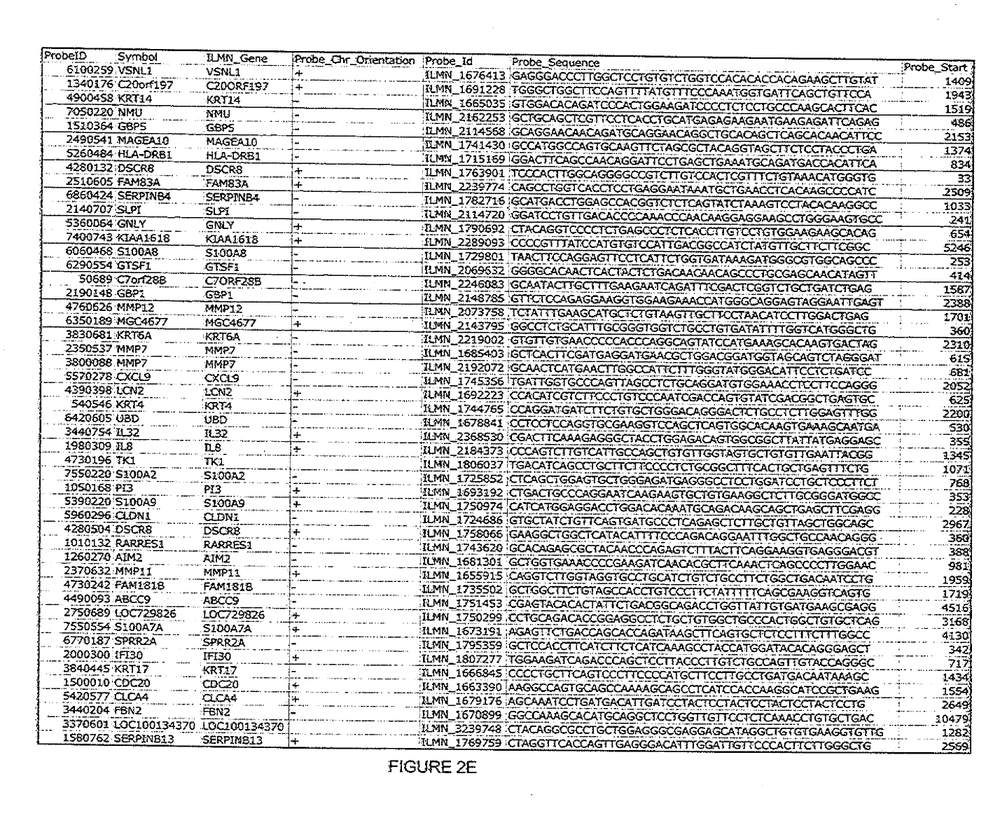

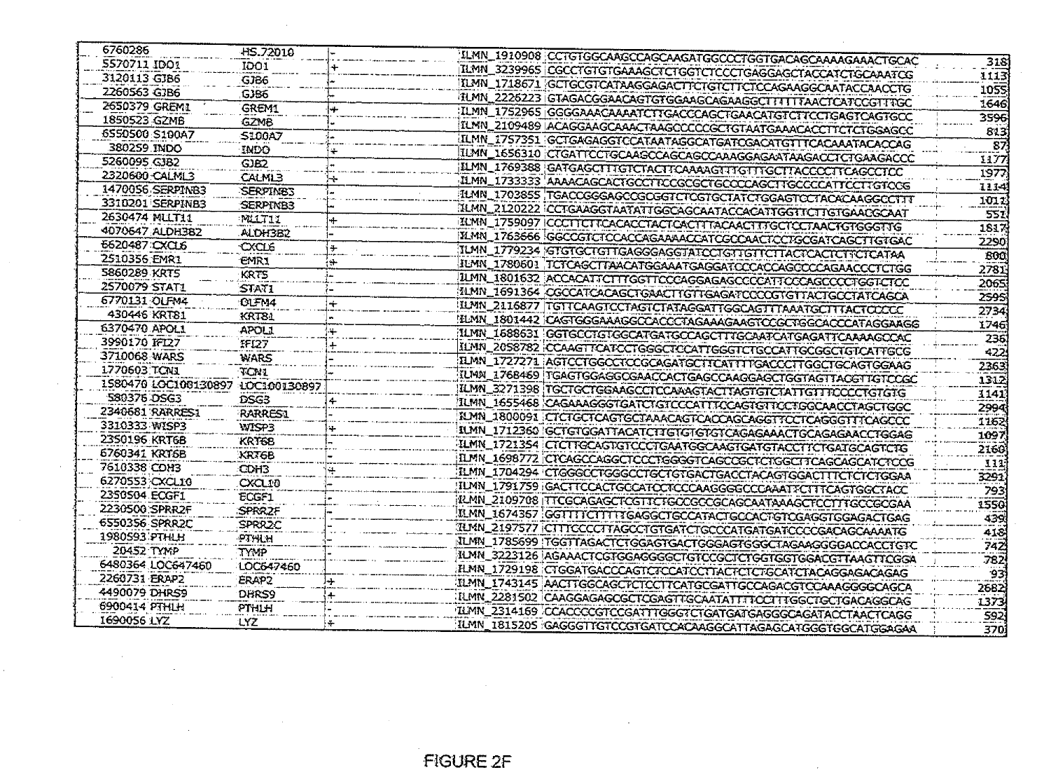

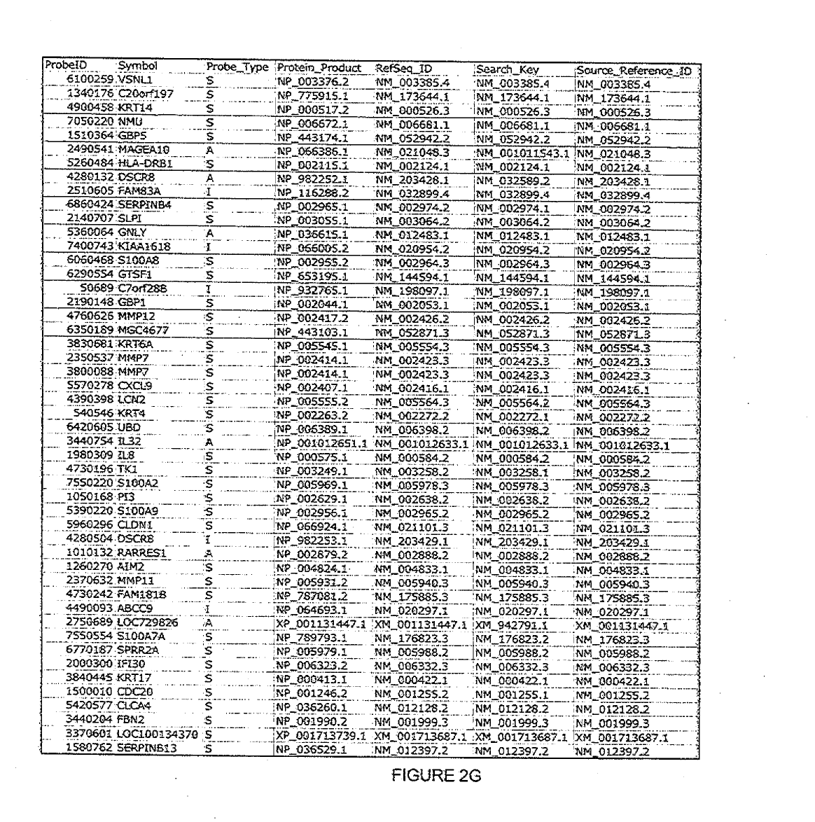

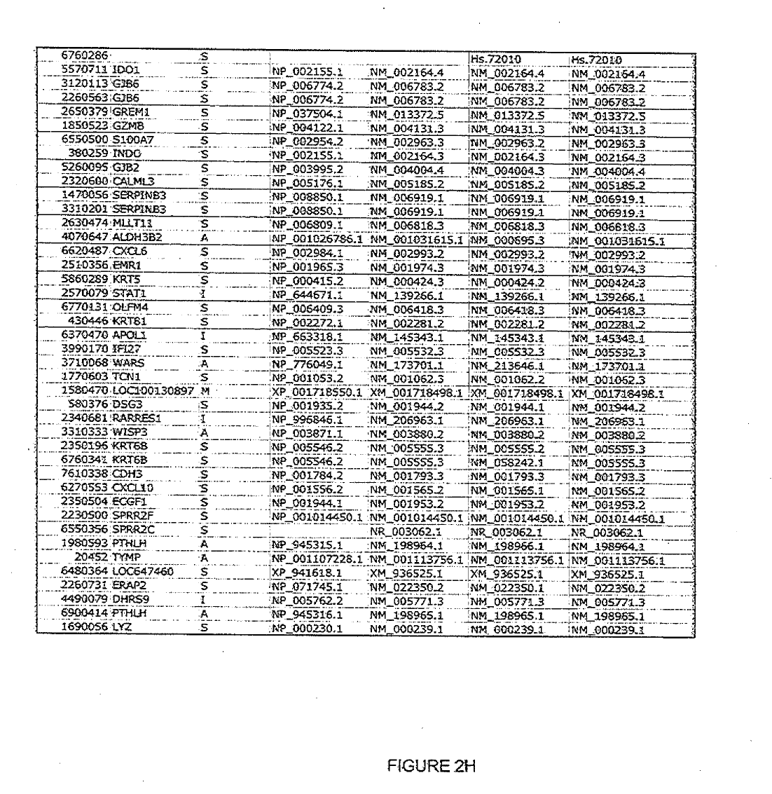

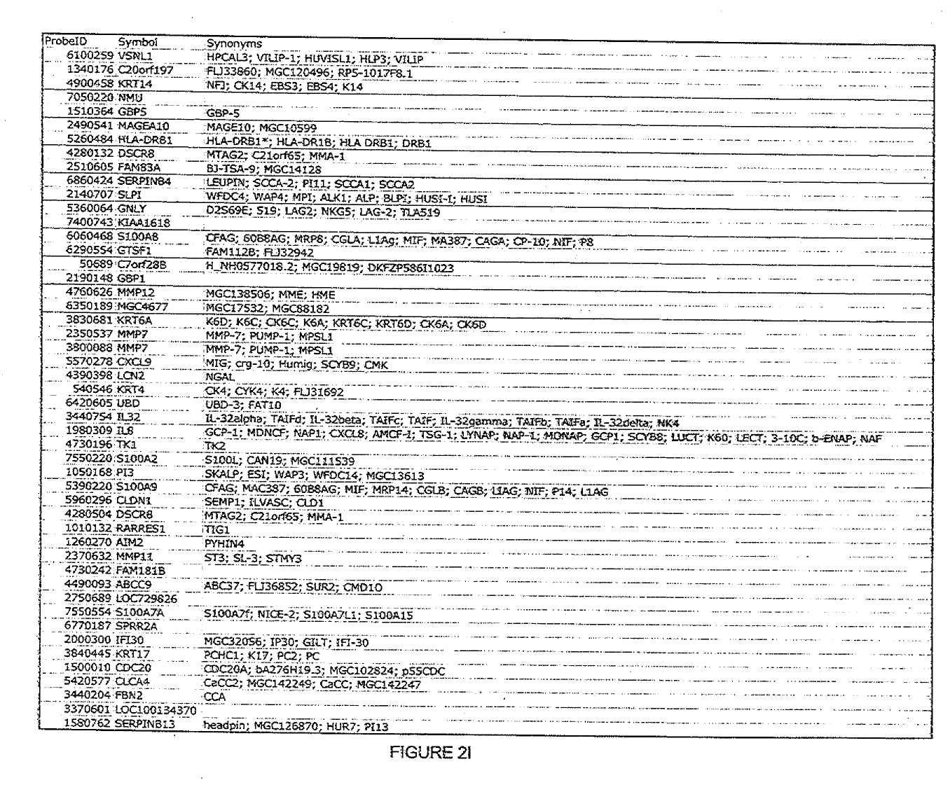

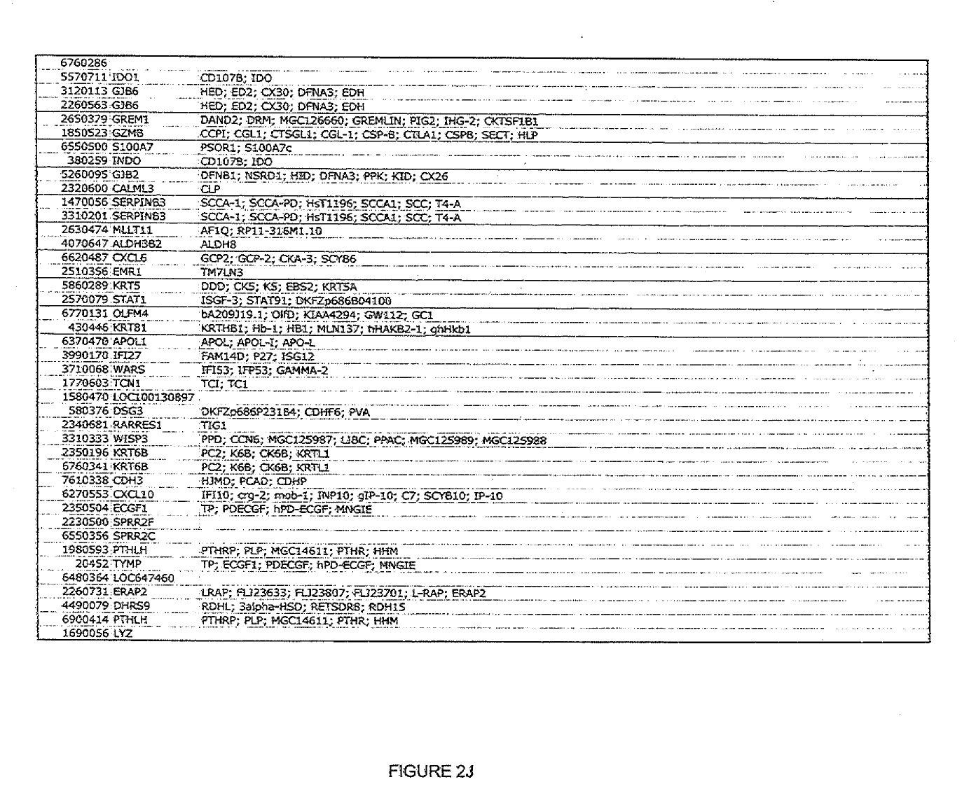

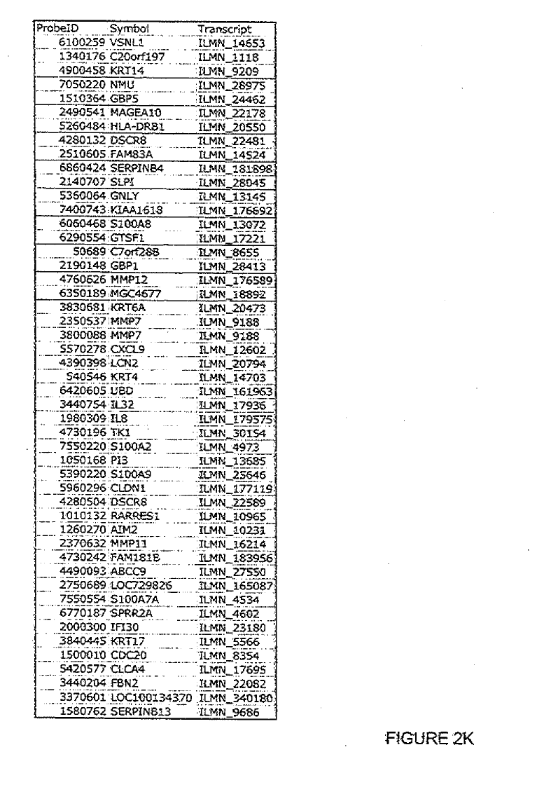

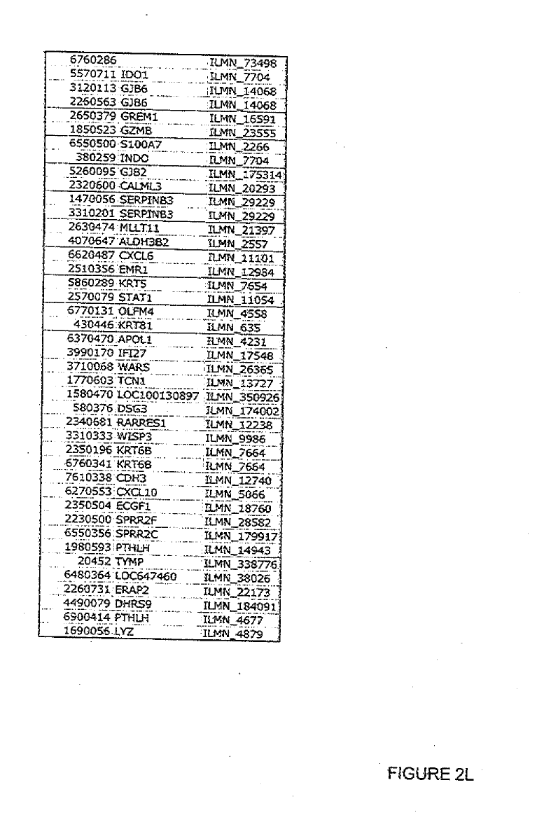

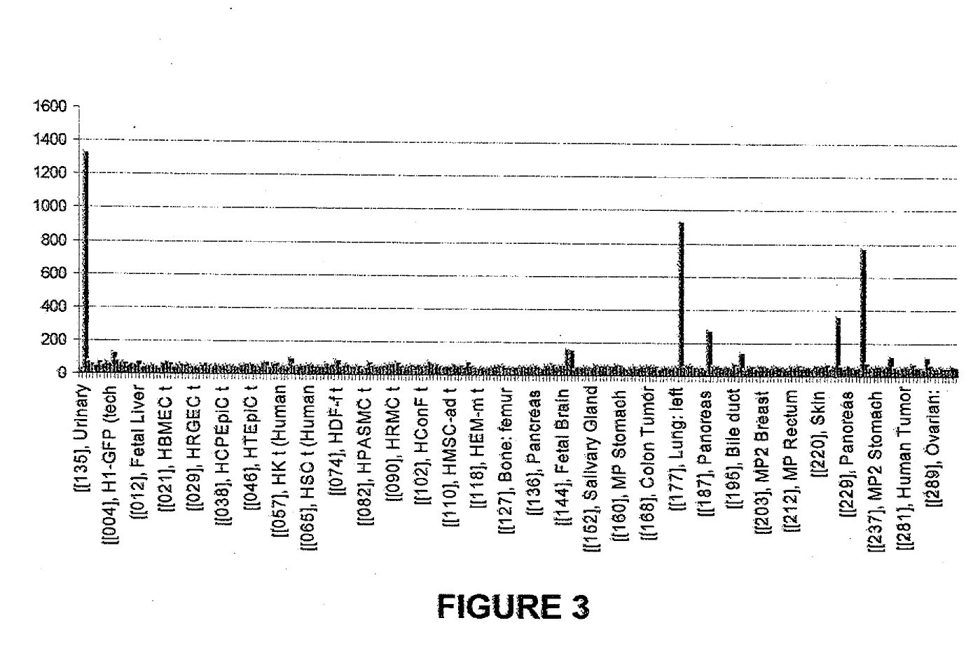

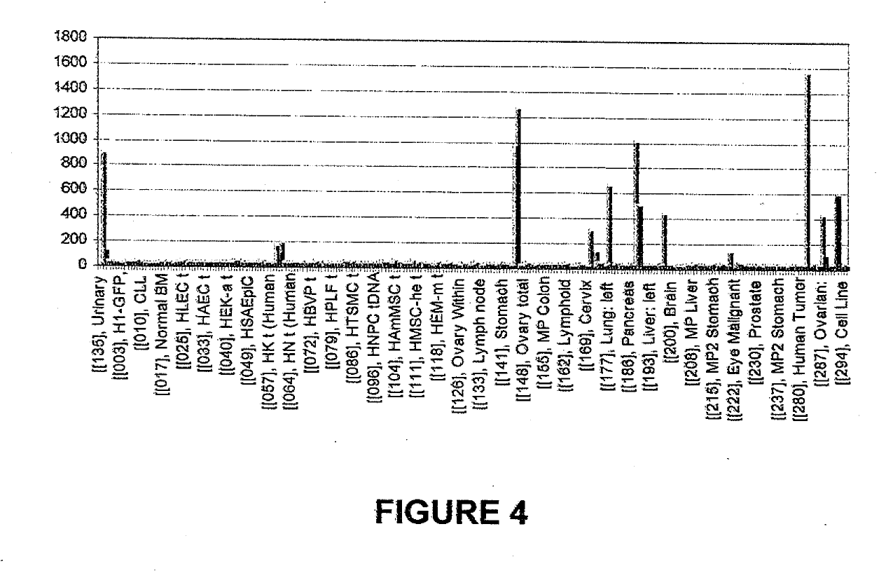

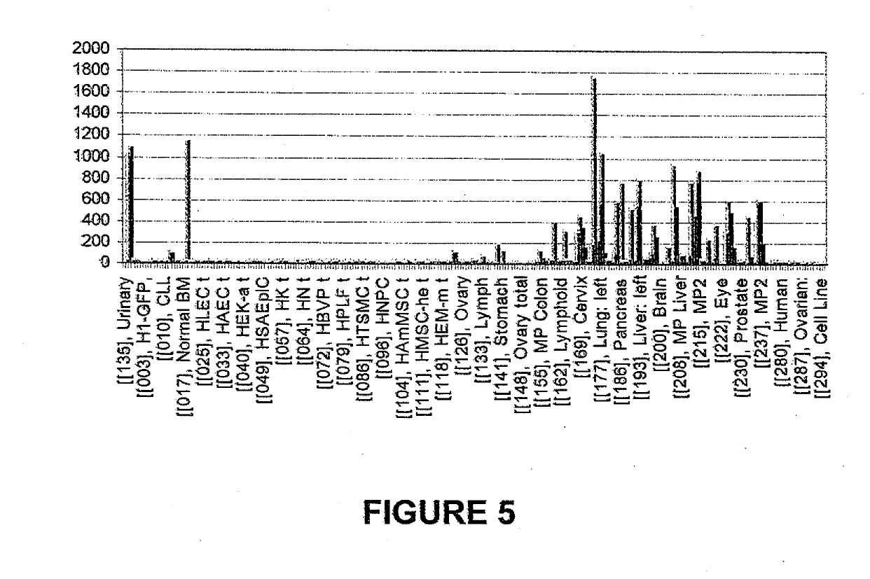

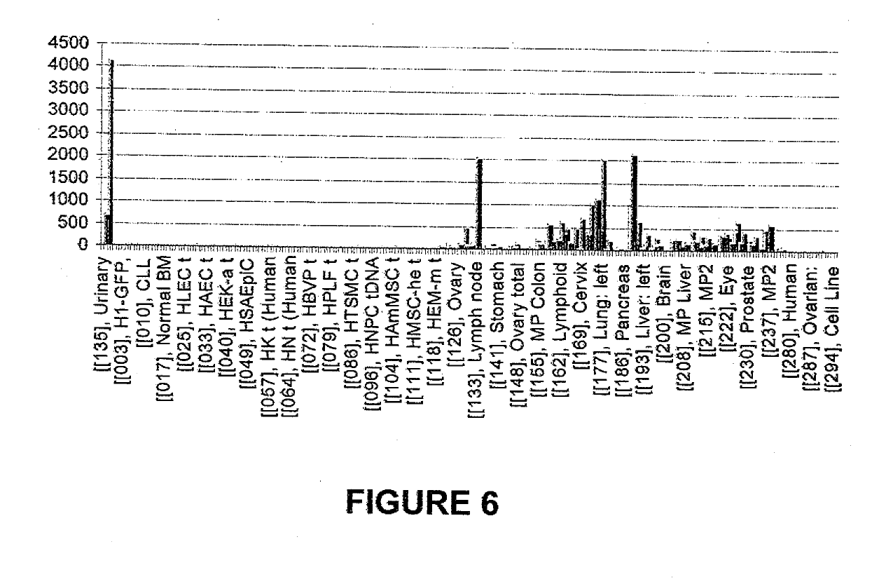

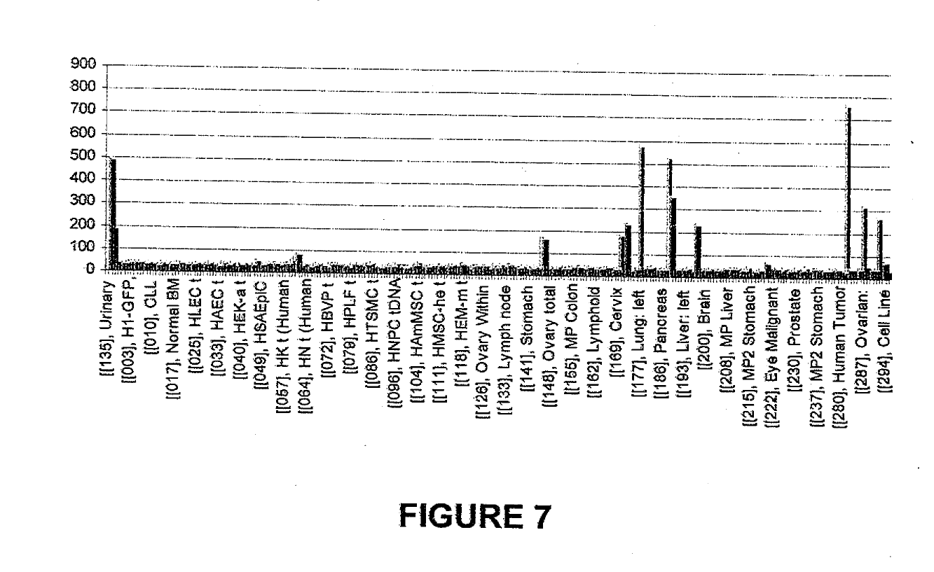

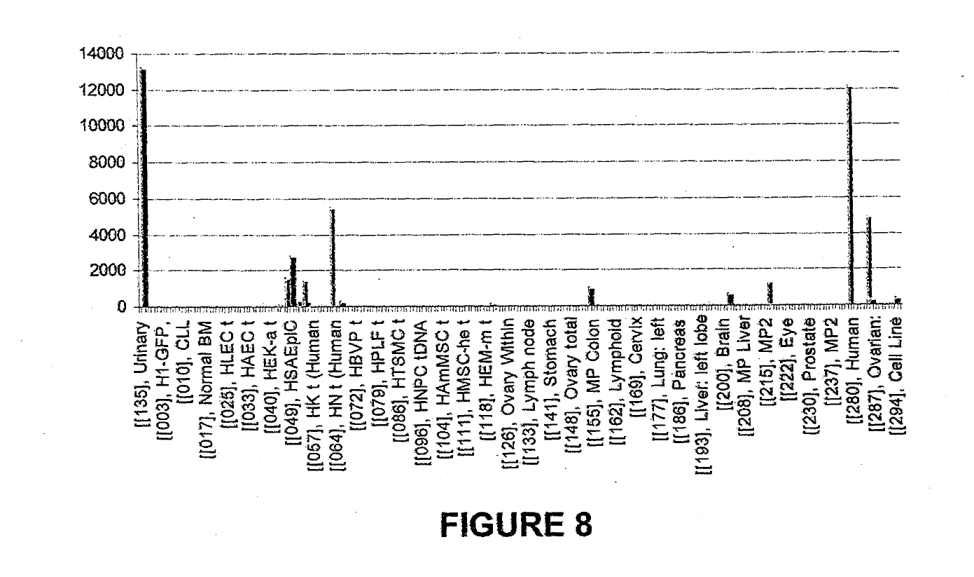

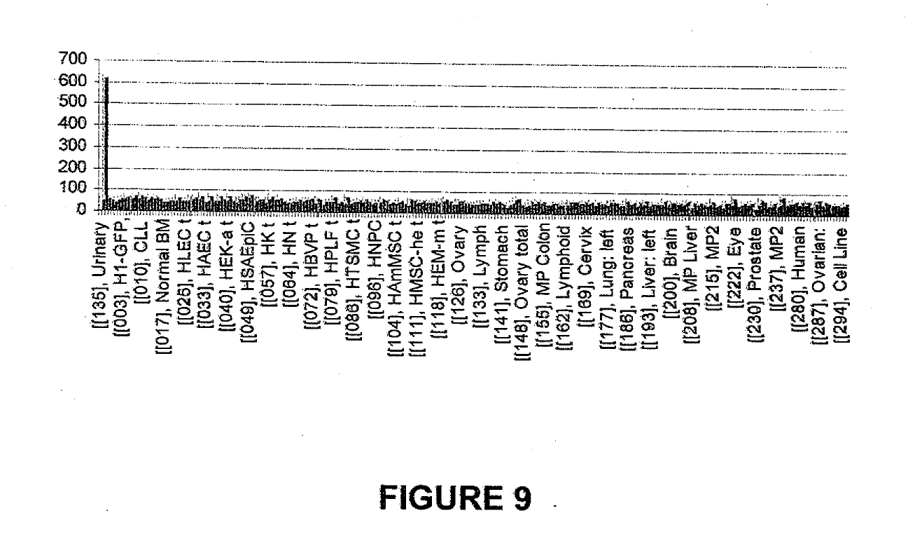

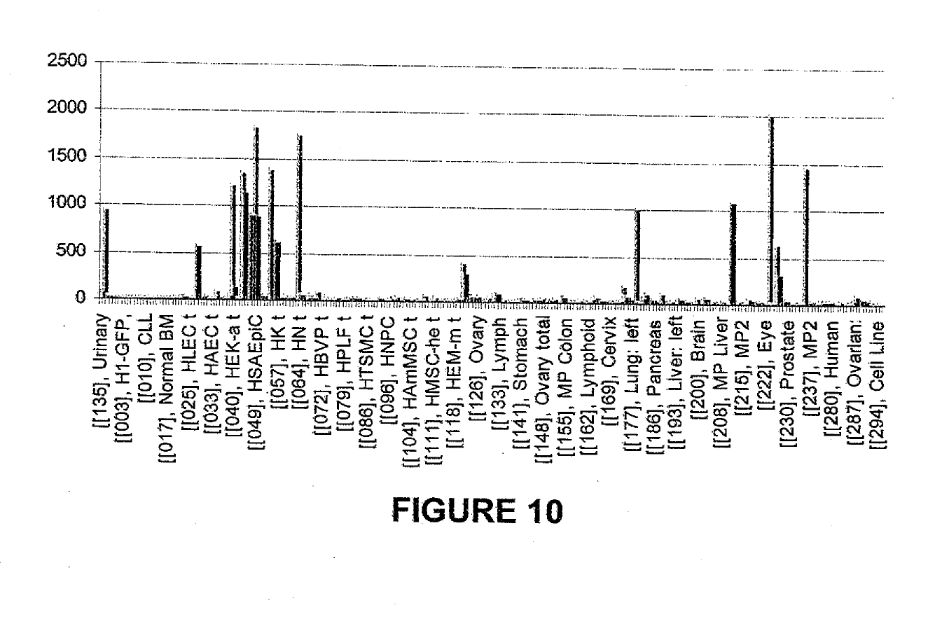

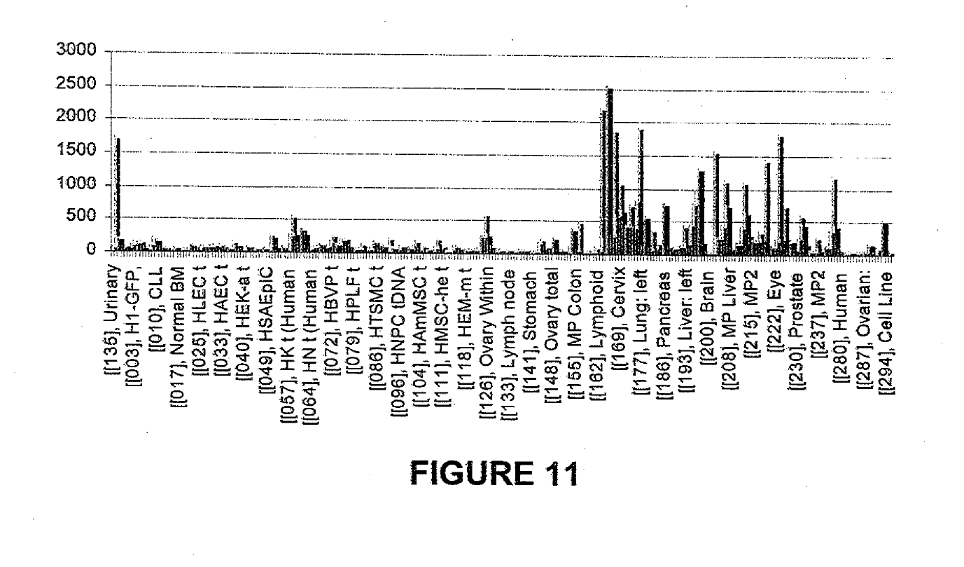

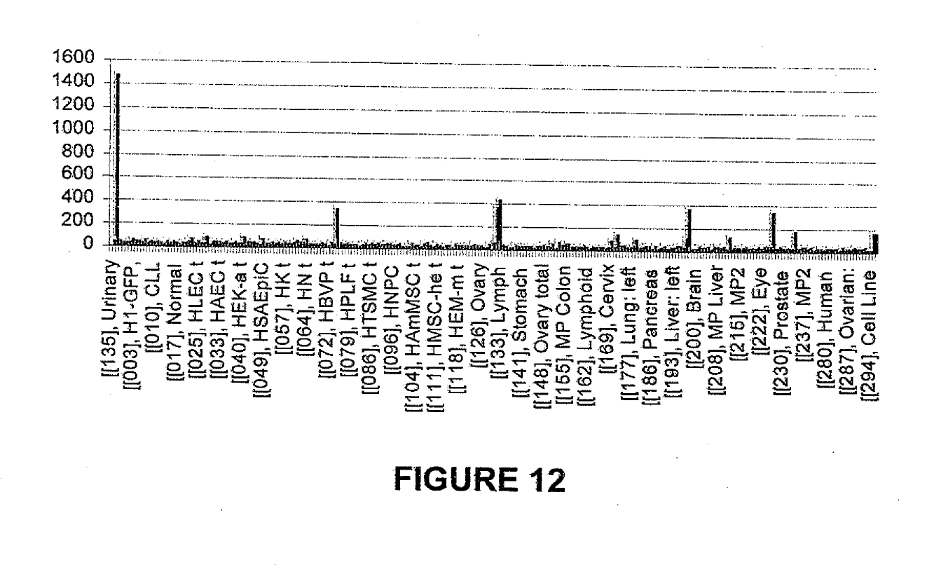

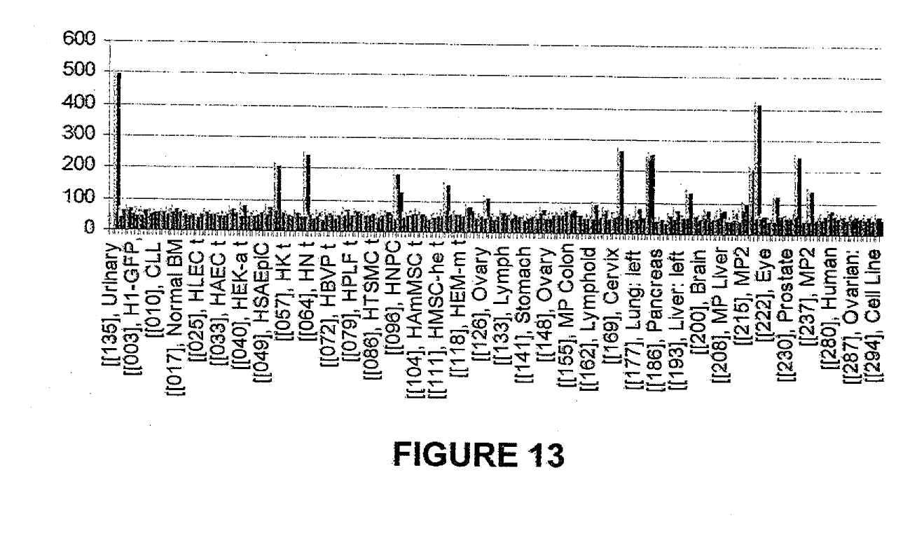

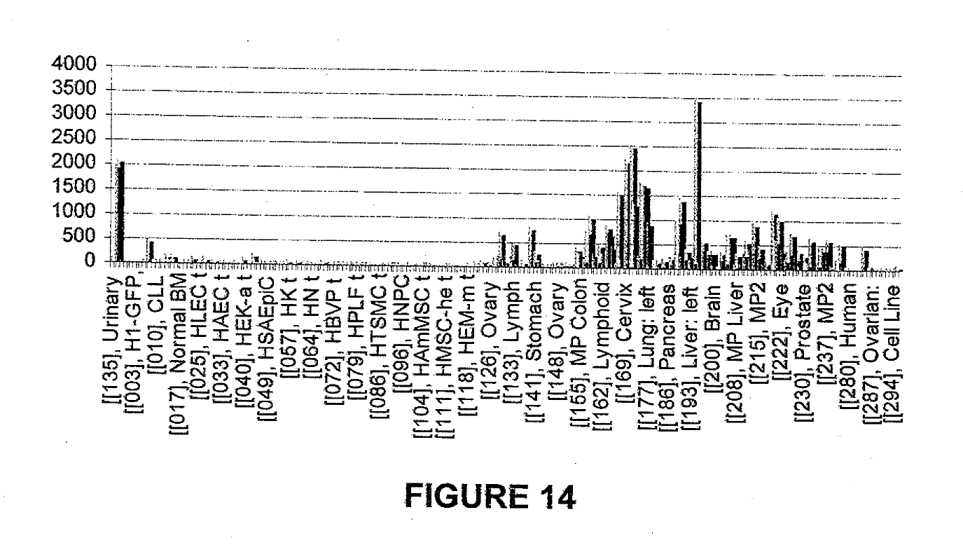

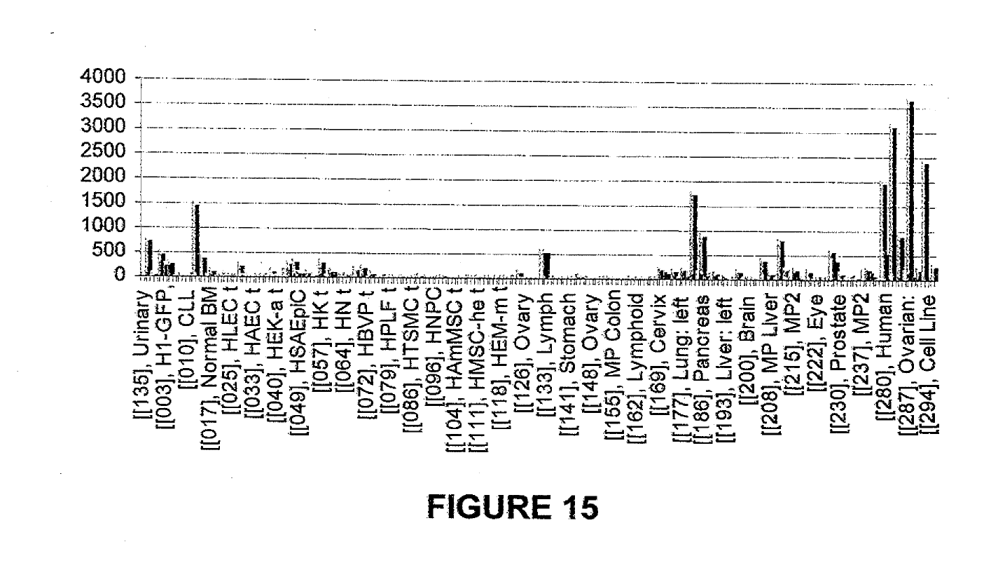

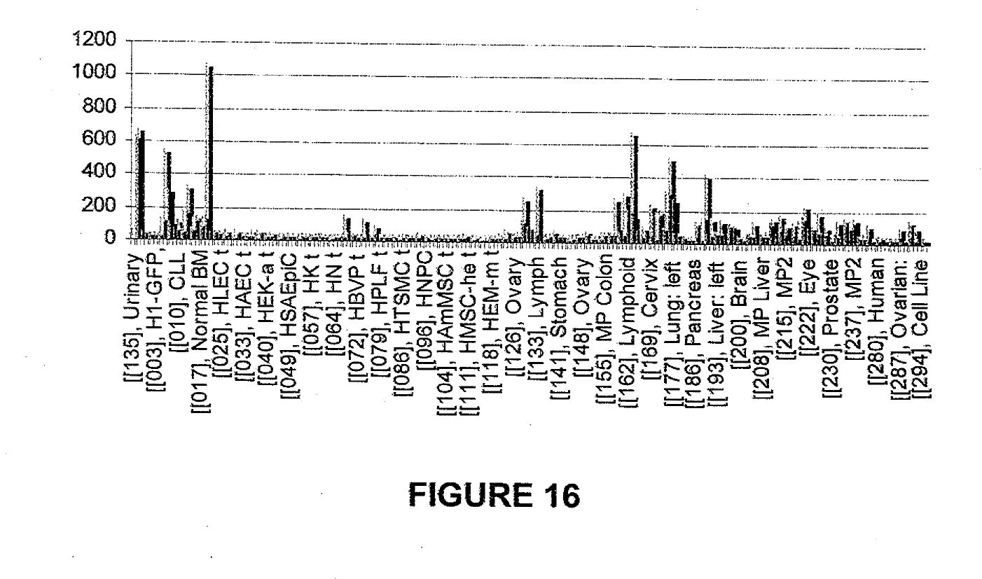

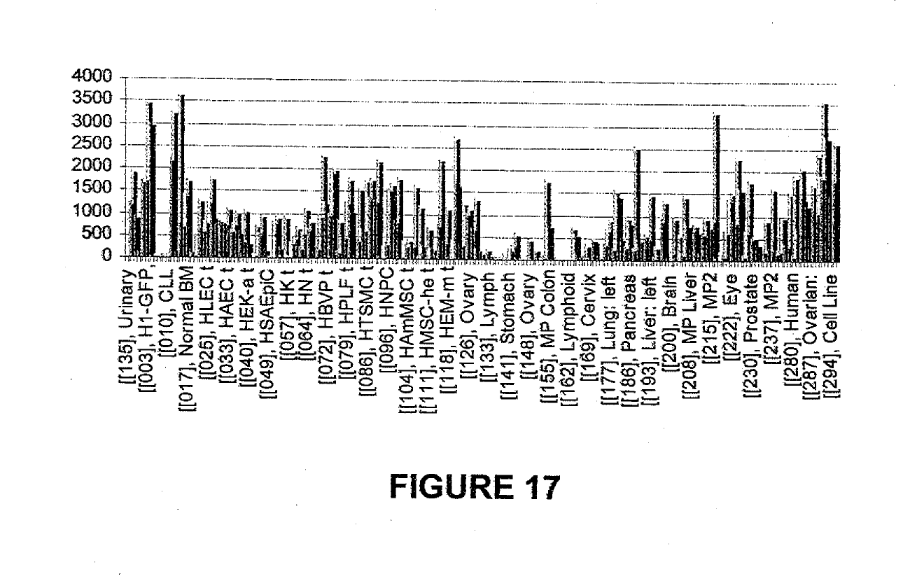

### DETAILED DESCRIPTION

Before the present compositions and methods are described, it is to be understood that this invention is not limited to the particular processes, compositions, or methodologies described, as these may vary. It is also to be understood that the terminology used in the description is for the purpose of describing the particular versions or embodiments only, and is not intended to limit the scope of the present invention which will be limited only by the appended claims. Unless defined otherwise, all technical and scientific terms used herein have the same meanings as commonly understood by one of ordinary skill in the art. Although any methods and materials similar or equivalent to those described herein can be used in the practice or testing of embodiments of the present invention, specific methods, devices, and materials are now described.

The invention provides for the rapid, accurate, and cost effective means to detect bladder cancer in a subject. The method comprises detecting one or more markers that are specifically expressed on bladder cancer tumors in a sample as disclosed infra. The sample may be a bodily fluid such as serum, or urine. Thus in some embodiments the invention provides for a non-invasive test for detecting bladder cancer in a subject. In other embodiments the sample may be a tissue or cell sample. Also provided are methods of screening for drugs having activity against bladder cancer, therapeutics for bladder cancer as well as compositions and kits useful in detecting, and prognosing bladder cancer.

### DEFINITIONS

As used herein, the singular forms “a,” “an,” and “the” include plural reference unless the context clearly dictates otherwise. Thus, for example, reference to a “therapeutic” is a reference to one or more therapeutics and equivalents thereof known to those skilled in the art, and so forth.

As used herein, the term “about” means plus or minus 10% of the numerical value of the number with which it is being used. Therefore, about 50% means in the range of 45% to 55%.

“Administering,” when used in conjunction with a therapeutic, means to administer a therapeutic directly into or onto a target tissue or to administer a therapeutic to a patient whereby the therapeutic positively impacts the tissue to which it is targeted. Thus, as used herein, the term “administering”, when used in conjunction with elastin digest, can include, but is not limited to, providing an elastin digest into or onto the target tissue; providing an elastin digest systemically to a patient by, e.g., intravenous injection whereby the therapeutic reaches the target tissue; providing an elastin digest in the form of the encoding sequence thereof to the target tissue (e.g., by so-called gene-therapy techniques). “Administering” a composition may be accomplished by oral administration, intravenous injection, intraperitoneal injection, intramuscular injection, subcutaneous injection, transdermal diffusion or electrophoresis, local injection, extended release delivery devices including locally implanted extended release devices such as bioerodible or reservoir-based implants, as protein therapeutics or as nucleic acid therapeutic via gene therapy vectors, topical administration, or by any of these methods in combination with other known techniques. Such combination techniques include heating, radiation and ultrasound.

The term “animal,” “patient” or “subject” as used herein includes, but is not limited to mammals, including humans and non-human primates, farm animals such as pigs, goats, horses, sheep, cows, rodents including rats and mice, rabbits, cats, dogs and the like. In some embodiments, the term “subject,” may refer to humans. In some embodiments, the term “subject,” may refer to a male. In some embodiments, the term “subject,” may refer to a female.

The term “bladder cancer” as used herein, may include transitional cell carcinoma, squamous cell carcinoma, adenocarcinoma, sarcoma, small cell carcinoma, secondary deposits from cancers elsewhere in the body or a combination thereof.

The term “inhibiting” includes the administration of a compound of the present invention to prevent the onset of the symptoms, alleviating the symptoms, or eliminating the disease, condition or disorder.

By “pharmaceutically acceptable”, it is meant the carrier, diluent or excipient must be compatible with the other ingredients of the formulation and not deleterious to the recipient thereof.

In some embodiments, the present disclosure provides for nucleic acid and protein sequences that are associated with cancer, herein termed “cancer associated” or “CA” sequences. In some embodiments, the present disclosure provides nucleic acid and protein sequences that are associated with cancers or carcinomas that originate in bladder or urinary tissue, herein termed “bladder cancer associated” sequences.

The term “pluripotent stem cells” refers to animal cells capable of differentiating into more than one differentiated cell type. Such cells include hES cells, hED cells, hEG cells, hEC cells, and adult-derived cells including mesenchymal stem cells, neuronal stem cells, and bone marrow-derived stem cells. Pluripotent stem cells may be genetically modified or not genetically modified. Genetically modified cells may include markers such as fluorescent proteins to facilitate their identification.

The term “embryonic stem cells” (ES cells) refers to cells derived from the inner cell mass of blastocysts, blastomeres, or morulae that have been serially passaged as cell lines while maintaining an undifferentiated state (e.g. expressing TERT, OCT4, and SSEA and TRA antigens specific for ES cells of the species). Established cell lines may be available from cell banks such as WiCell. The ES cells may be derived from in vitro fertilization of an egg cell with sperm or DNA, nuclear transfer, parthenogenesis, or by means to generate hES cells with hemizygosity or homozygosity in the MHC region. The term “human embryonic stem cells” (hES cells) refers to human ES cells.

The term “human embryonic germ cells” (hEG cells) refer to pluripotent stem cells derived from the primordial germ cells of fetal tissue or maturing or mature germ cells such as oocytes and spermatogonial cells, that can differentiate into various tissues in the body. The hEG cells may also be derived from pluripotent stem cells produced by gynogenetic or androgenetic means, i.e., methods wherein the pluripotent cells are derived from oocytes containing only DNA of male or female origin and therefore will comprise all female-derived or male-derived DNA (see U.S. application Nos. 60/161,987, filed Oct. 28, 1999; Ser. No. 09/697,297, filed Oct. 27, 2000; Ser. No. 09/995,659, filed Nov. 29, 2001; Ser. No. 10/374,512, filed Feb. 27, 2003; PCT application no. PCT/US/00/29551, filed Oct. 27, 2000; the disclosures of which are incorporated herein in their entirety).

The term human iPS cells refers to cells with properties similar to hES cells, including the ability to form all three germ layers when transplanted into immunocompromised mice wherein said iPS cells are derived from cells of varied somatic cell lineages following exposure to hES cell-specific transcription factors such as KLF4, SOX2, MYC, and OCT4 or the factors SOX2, OCT4, NANOG, and LIN28. Said iPS cells may be produced by the expression of these gene through vectors such as retroviral vectors as is known in the art, or through the introduction of these factors by permeabilization or other technologies taught by PCT application number PCT/US2006/030632 (WO2007/019398).

The term “differentiated cells” when used in reference to cells made by methods of this invention from pluripotent stem cells refer to cells having reduced potential to differentiate when compared to the parent pluripotent stem cells. The differentiated cells of this invention comprise cells that could differentiate further (i.e., they may not be terminally differentiated).

The term embryonal carcinoma (“EC”) cells, including human EC cells, refers to embryonal carcinoma cells such as TERA-1, TERA-2, and NTera-2.

As used herein, the term “naturally occurring” refers to sequences or structures that may be in a form normally found in nature. “Naturally occurring” may include sequences in a form normally found in any animal.

As used herein, the term “cancer associated sequences” refers to nucleotide or protein sequences that are either differentially expressed, activated, inactivated or altered in cancers as compared to normal tissue. Cancer associated sequences may include those that are up-regulated (i.e. expressed at a higher level), as well as those that are down-regulated (i.e. expressed at a lower level), in cancers when compared to a non-cancerous or normal sample. Cancer associated sequences can also include sequences that have been altered (i.e., translocations, truncated sequences or sequences with substitutions, deletions or insertions, including, but not limited to, point mutations) and show either the same expression profile or an altered profile. In some embodiments, the cancer associated sequences are from humans; however, as will be appreciated by those in the art, cancer associated sequences from other subjects may be useful in animal models of disease and drug evaluation; thus, other cancer associated sequences may be useful such as any subject, e.g., without limitation, sequences from vertebrates, including mammals, including rodents (rats, mice, hamsters, guinea pigs, etc.), primates, and farm animals (including sheep, goats, pigs, cows, horses, etc). Cancer associated sequences from other organisms may be obtained using the techniques outlined below.

The term “homology,” as used herein, refers to a degree of complementarity. There may be partial homology or complete homology. The word “identity” may substitute for the word “homology.” A partially complementary nucleic acid sequence that at least partially inhibits an identical sequence from hybridizing to a target nucleic acid is referred to as “substantially homologous.” The inhibition of hybridization of the completely complementary sequence to the target sequence may be examined using a hybridization assay (Southern or northern blot, solution hybridization, and the like) under conditions of reduced stringency. A substantially homologous sequence or hybridization probe will compete for and inhibit the binding of a completely homologous sequence to the target sequence under conditions of reduced stringency. This is not to say that conditions of reduced stringency are such that non-specific binding is permitted, as reduced stringency conditions require that the binding of two sequences to one another be a specific (i.e., a selective) interaction. The absence of non-specific binding may be tested by the use of a second target sequence which lacks even a partial degree of complementarity (e.g., less than about 30% homology or identity). In the absence of non-specific binding, the substantially homologous sequence or probe will not hybridize to the second non-complementary target sequence.

The phrases “percent homology,” “% homology,” “percent identity” or “% identity” refer to the percentage of sequence similarity found in a comparison of two or more amino acid or nucleic acid sequences. Percent identity can be determined electronically, e.g., by using the MEGALIGN program (LASERGENE software package, DNASTAR). The MEGALIGN program can create alignments between two or more sequences according to different methods, e.g., the Clustal Method. (Higgins, D. G. and P. M. Sharp (1988) Gene 73:237-244.) The Clustal algorithm groups sequences into clusters by examining the distances between all pairs. The clusters are aligned pairwise and then in groups. The percentage similarity between two amino acid sequences, e.g., sequence A and sequence B, is calculated by dividing the length of sequence A, minus the number of gap residues in sequence A, minus the number of gap residues in sequence B, into the sum of the residue matches between sequence A and sequence B, times one hundred. Gaps of low or of no homology between the two amino acid sequences are not included in determining percentage similarity. Percent identity between nucleic acid sequences can also be calculated by the Clustal Method, or by other methods known in the art, such as the Jotun Hein Method. (See, e.g., Hein, J. (1990) Methods Enzymol. 183:626-645.) Identity between sequences can also be determined by other methods known in the art, e.g., by varying hybridization conditions.

As used herein, a polynucleotide “derived from” a designated sequence refers to a polynucleotide sequence which is comprised of a sequence of approximately at least about 6 nucleotides, at least about 8 nucleotides, at least about 10-12 nucleotides, and at least about 15-20 nucleotides corresponding to a region of the designated nucleotide sequence. “Corresponding” means homologous to or complementary to the designated sequence. Preferably, the sequence of the region from which the polynucleotide is derived is homologous to or complementary to a sequence that is unique to a cancer associated gene.

In the broadest sense, use of “nucleic acid,” “polynucleotide” or “oligonucleotide” or equivalents herein means at least two nucleotides covalently linked together. In some embodiments, an oligonucleotide is all oligomer of 6, 8, 10, 12, 20, 30 or up to 100 nucleotides. In some embodiments, an oligonucleotide is an oligomer of at least 6, 8, 10, 12, 20, 30, 40, 50, 60, 70, 80, 90, 100, 150, 200, 300, 400, or 500 nucleotides. A “polynucleotide” or “oligonucleotide” may comprise DNA, RNA, PNA or a polymer of nucleotides linked by phosphodiester and/or any alternate bonds. The nucleic acid, polynucleotide or oligonucleotide may be modified by linking a detectable substance or label to it.

Similarly, a “recombinant protein” is a protein made using recombinant techniques, for example, but not limited to, through the expression of a recombinant nucleic acid as depicted above. A recombinant protein may be distinguished from naturally occurring protein by at least one or more characteristics. For example, the protein may be isolated or purified away from some or all of the proteins and compounds with which it is normally associated in its wild type host, and thus may be substantially pure. For example, an isolated protein is unaccompanied by at least some of the material with which it is normally associated in its natural state, preferably constituting at least about 0.5%, more preferably at least about 5% by weight of the total protein in a given sample. A substantially pure protein comprises about 50-75%, about 80%, or 90% by weight of the total protein. In some embodiments, a substantially pure protein comprises about 80-99%, 85-99%, 90-99%, 95-99%, or 97-99% by weight of the total protein. A recombinant protein can also include the production of a cancer associated protein from one organism (e.g. human) in a different organism (e.g. yeast, E. coli, and the like) or host cell (e.g. yeast, E. coli, and the like). Alternatively, the protein may be made at a significantly higher concentration than is normally seen, through the use of an inducible promoter or high expression promoter, such that the protein is made at increased concentration levels. Alternatively, the protein may be in a form not normally found in nature, as in the addition of an epitope tag e.g. a detectable substance or label, or amino acid substitutions, insertions and deletions, as discussed herein.

As used herein, the term “tag,” “sequence tag” or “primer tag sequence” refers to an oligonucleotide with specific nucleic acid sequence that serves to identify a batch of polynucleotides bearing such tags therein. Polynucleotides from the same biological source are covalently tagged with a specific sequence tag so that in subsequent analysis the polynucleotide can be identified according to its source of origin. The sequence tags also serve as primers for nucleic acid amplification reactions.

A “microarray” is a linear or two-dimensional array of, for example, discrete regions, each having a defined area, formed on the surface of a solid support. The density of the discrete regions on a microarray is determined by the total numbers of target polynucleotides to be detected on the surface of a single solid phase support, preferably at least about 50/cm², more preferably at least about 100/cm², even more preferably at least about 500/cm², and still more preferably at least about 1,000/cm². As used herein, a DNA microarray is an array of oligonucleotide primers placed on a chip or other surfaces used to identify, amplify, detect, or clone target polynucleotides. Since the position of each particular group of primers in the array is known, the identities of the target polynucleotides can be determined based on their binding to a particular position in the microarray.

The term “label” or “detectable substance” refers to a composition capable of producing a detectable signal indicative of the presence of the target polynucleotide in an assay sample. Suitable labels include radioisotopes, nucleotide chromophores, enzymes, substrates, fluorescent molecules, chemiluminescent moieties, magnetic particles, bioluminescent moieties, and the like. As such, a label is any composition detectable by a device or method, such as but not limited to, a spectroscopic, photochemical, biochemical, immunochemical, electrical, optical, chemical detection device or any other appropriate device. The label can also be detectable visually without the aid of a device. The term “label” is used to refer to any chemical group or moiety having a detectable physical property or any compound capable of causing a chemical group or moiety to exhibit a detectable physical property, such as an enzyme that catalyzes conversion of a substrate into a detectable product. The term “label” also encompasses compounds that inhibit the expression of a particular physical property. The label may also be a compound that is a member of a binding pair, the other member of which bears a detectable physical property.

The term “support” refers to conventional supports such as beads, particles, dipsticks, fibers, filters, membranes, and silane or silicate supports such as glass slides.

The term “amplify” is used in the broad sense to mean creating an amplification product which may include, for example, additional target molecules, or target-like molecules or molecules complementary to the target molecule, which molecules are created by virtue of the presence of the target molecule in the sample. In the situation where the target is a nucleic acid, an amplification product can be made enzymatically with DNA or RNA polymerases or reverse transcriptases, or any combination thereof.

As used herein, a “biological sample” refers to a sample of tissue or fluid isolated from a subject, including but not limited to, for example, blood, plasma, serum, spinal fluid, lymph fluid, skin, respiratory, intestinal and genitourinary tracts, tears, saliva, milk, cells (including but not limited to blood cells), tumors, organs, and also samples of in vitro cell culture constituents.

The term “biological sources” as used herein refers to the sources from which the target polynucleotides may be derived. The source can be of any form of “sample” as described above, including but not limited to, cell, tissue or fluid. “Different biological sources” can refer to different cells/tissues/organs of the same individual, or cells/tissues/organs from different individuals of the same species, or cells/tissues/organs from different species.

As used herein, the term “therapeutic” or “therapeutic agent” means an agent that can be used to treat, combat, ameliorate, prevent or improve an unwanted condition or disease of a patient. In part, embodiments of the present invention are directed to the treatment of cancer or the decrease in proliferation of cells. In some embodiments, the term “therapeutic” or “therapeutic agent” may refer to any molecule that associates with or affects the target marker, its expression or its function. In various embodiments, such therapeutics may include molecules such as, for example, a therapeutic cell, a therapeutic peptide, a therapeutic gene, a therapeutic compound, or the like, that associates with or affects the target marker, its expression or its function.

A “therapeutically effective amount” or “effective amount” of a composition is a predetermined amount calculated to achieve the desired effect, i.e., to inhibit, block, or reverse the activation, migration, or proliferation of cells. In some embodiments, the effective amount is a prophylactic amount. In some embodiments, the effective amount is an amount used to medically treat the disease or condition. The specific dose of a composition administered according to this invention to obtain therapeutic and/or prophylactic effects will, of course, be determined by the particular circumstances surrounding the case, including, for example, the composition administered, the route of administration, and the condition being treated. It will be understood that the effective amount administered will be determined by the physician in the light of the relevant circumstances including the condition to be treated, the choice of composition to be administered, and the chosen route of administration. A therapeutically effective amount of composition of this invention is typically an amount such that when it is administered in a physiologically tolerable composition, it is sufficient to achieve an effective systemic concentration or local concentration in the targeted tissue.

The terms “treat,” “treated,” or “treating” as used herein can refer to both therapeutic treatment or prophylactic or preventative measures, wherein the object is to prevent or slow down (lessen) an undesired physiological condition, disorder or disease, or to obtain beneficial or desired clinical results. In some embodiments, the term may refer to both treating and preventing. For the purposes of this disclosure, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects. Treatment also includes prolonging survival as compared to expected survival if not receiving treatment. Treat, treated, or treating may include inhibiting the growth a bladder cancer tumor and/or inhibiting the metastasis of a bladder cancer tumor.

Generally speaking, the term “tissue” refers to any aggregation of similarly specialized cells that are united in the performance of a particular function.

“Optional” or “optionally” means that the subsequently described structure, event or circumstance may or may not occur, and that the description includes instances where the event occurs and instances where it does not.

### Cancer Associated Nucleic Acid Sequences and Cancer Detection

Some embodiments herein are directed to one or more of sequences associated with cancers, such as, bladder cancer. A list of genes associated with bladder cancer is provided in Table 2 and the corresponding nucleic acid sequences are provided in Table 5.

In some embodiments, the cancer associated sequences are nucleic acids. As will be appreciated by those skilled in the art and is described herein, cancer associated sequences of embodiments herein may be useful in a variety of applications including diagnostic applications to detect nucleic acids or their expression levels in a subject, therapeutic applications or a combination thereof. Further, the cancer associated sequences of embodiments herein may be used in screening applications; for example, generation of biochips comprising nucleic acid probes to the cancer associated sequences.

In some embodiments, cancer associated sequences may include nucleic acid and/or amino acid sequences. In some embodiments, the cancer associated sequences may include sequences having at least about 60% homology with the disclosed sequences. In some embodiments, the cancer associated sequences may have at least about 65%, about 70%, about 75%, about 80%, about 85%, about 90%, about 95%, about 97%, about 99%, about 99.8% homology with the disclosed sequences. In some embodiments, the cancer associated sequences may be “mutant nucleic acids”. As used herein, “mutant nucleic acids” refers to deletion mutants, insertions, point mutations, substitutions, translocations.

A nucleic acid of the present invention may include phosphodiester bonds, although in some cases, as outlined below (for example, in antisense applications or when a nucleic acid is a candidate drug agent), nucleic acid analogs may have alternate backbones, comprising, for example, phosphoramidate (Beaucage et al., Tetrahedron 49(10):1925 (1993) and references therein; Letsinger, J. Org. Chem. 35:3800 (1970); Sprinzl et al., Eur. J. Biochem. 81:579 (1977); Letsinger et al., Nucl. Acids Res. 14:3487 (1986); Sawai et al., Chem. Lett. 805 (1984), Letsinger et al., J. Am. Chem. Soc. 110:4470 (1988); and Pauwels et al., Chemica Scripta 26:141 91986)), phosphorothioate (Mag et al., Nucleic Acids Res. 19:1437 (1991); and U.S. Pat. No. 5,644,048), phosphorodithioate (Briu et al., J. Am. Chem. Soc. 111:2321 (1989), O-methylphosphoroamidite linkages (see Eckstein, Oligonucleotides and Analogues: A Practical Approach, Oxford University Press), and peptide nucleic acid backbones and linkages (see Egholm, J. Am. Chem. Soc. 114:1895 (1992); Meier et al., Chem. Int. Ed. Engl. 31:1008 (1992); Nielsen, Nature, 365:566 (1993); Carlsson et al., Nature 380:207 (1996), all of which are incorporated by reference). Other analog nucleic acids include those with positive backbones (Denpcy et al., Proc. Natl. Acad. Sci. USA 92:6097 (1995); non-ionic backbones (U.S. Pat. Nos. 5,386,023, 5,637,684, 5,602,240, 5,216,141 and 4,469,863; Kiedrowshi et al., Angew. Chem. Intl. Ed. English 30:423 (1991); Letsinger et al., J. Am. Chem. Soc. 110:4470 (1988); Letsinger et al., Nucleoside & Nucleotide 13:1597 (1994); Chapters 2 and 3, ASC Symposium Series 580, “Carbohydrate Modifications in Antisense Research”, Ed. Y. S. Sanghui and P. Dan Cook; Mesmaeker et al., Bioorganic & Medicinal Chem. Lett. 4:395 (1994); Jeffs et al., J. Biomolecular NMR 34:17 (1994); Tetrahedron Lett. 37:743 (1996)) and non-ribose backbones, including those described in U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook. Nucleic acids containing one or more carbocyclic sugars are also included within one definition of nucleic acids (see Jenkins et al., Chem. Soc. Rev. (1995) pp 169-176). Several nucleic acid analogs are described in Rawls, C & E News Jun. 2, 1997 page 35. These modifications of the ribose-phosphate backbone may be done for a variety of reasons, for example to increase the stability and half-life of such molecules in physiological environments for use in anti-sense applications or as probes on a biochip.

As will be appreciated by those skilled in the art, such nucleic acid analogs can be used in some embodiments. In addition, mixtures of naturally occurring nucleic acids and analogs can be made; alternatively, mixtures of different nucleic acid analogs, and mixtures of naturally occurring nucleic acids and analogs may be made.

In some embodiments, the nucleic acids may be single stranded or double stranded or may contain portions of both double stranded or single stranded sequence. As will be appreciated by those skilled in the art, the depiction of a single strand also defines the sequence of the other strand; thus the sequences described herein also includes the complement of the sequence. The nucleic acid may be DNA, both genomic and cDNA, RNA, or a hybrid, where the nucleic acid contains any combination of deoxyribo- and ribo-nucleotides, and any combination of bases, including uracil, adenine, thymine, cytosine, guanine, inosine, xanthine, hypoxanthine, isocytosine, isoguanine, etc. As used herein, the term “nucleoside” includes nucleotides and nucleoside and nucleotide analogs, and modified nucleosides such as amino modified nucleosides. In addition, “nucleoside” includes non-naturally occurring analog structures. Thus, for example, the subject units of a peptide nucleic acid, each containing a base, are referred to herein as a nucleoside.

In some embodiments, the cancer associated sequences may be recombinant nucleic acids. By the term “recombinant nucleic acid” herein refers to nucleic acid molecules, originally formed in vitro, in general, by the manipulation of nucleic acid by polymerases and endonucleases, in a form not normally found in nature. Thus a recombinant nucleic acid may also be an isolated nucleic acid, in a linear form, or cloned in a vector formed in vitro by ligating DNA molecules that are not normally joined, are both considered recombinant for the purposes of this invention. It is understood that once a recombinant nucleic acid is made and reintroduced into a host cell or organism, it can replicate using the in vivo cellular machinery of the host cell rather than in vitro manipulations; however, such nucleic acids, once produced recombinantly, although subsequently replicated in vivo, are still considered recombinant or isolated for the purposes of the invention. As used herein, a “polynucleotide” or “nucleic acid” is a polymeric form of nucleotides of any length, either ribonucleotides or deoxyribonucleotides. This term includes double- and single-stranded DNA and RNA. It also includes known types of modifications, for example, labels which are known in the art, methylation, “caps”, substitution of one or more of the naturally occurring nucleotides with an analog, internucleotide modifications—such as, for example, those with uncharged linkages (e.g., phosphorothioates, phosphorodithioates, etc.), those containing pendant moieties, such as, for example proteins (including e.g., nucleases, toxins, antibodies, signal peptides, poly-L-lysine, etc.), those with intercalators (e.g., acridine, psoralen, etc.), those containing chelators (e.g., metals, radioactive metals, etc.), those containing alkylators, those with modified linkages (e.g., alpha anomeric nucleic acids, etc.), as well as unmodified forms of the polynucleotide.

In some embodiments, a method of identifying a target marker comprises the steps of: 1) obtaining a molecular profile of the mRNAs, miRNAs, proteins, or protein modifications of immortal pluripotent stem cells (such as embryonic stem (“ES”) cells, induced pluripotent stem (“iPS”) cells, and germ-line cells such as embryonal carcinoma (“EC”) cells); 2) ES, iPS, or EC-derived clonal embryonic progenitor (“EP”) cell lines malignant cancer cells including cultured cancer cell lines or human tumor tissues, and comparing those molecules to those present in mortal somatic cell types such as cultured clonal human embryonic progenitors, cultured somatic cells from fetal or adult sources, or normal tissue counterparts to malignant cancer cells. Target markers that are shared between pluripotent stem cells such as hES cells and malignant cancer cells, but are not present in a majority of somatic cell types may be candidate diagnostic markers and therapeutic targets.

Some embodiments are directed to a biochip comprising a nucleic acid segment which encodes a cancer associated protein, for example, but not limited to, selected from the sequences outlined in Table 2 (SEQ ID NOs: 1-55).

Also provided herein is a method for diagnosing or determining the propensity to cancers, e.g., bladder cancer. The method of diagnosing may comprise measuring the level of expression of a cancer associated marker disclosed herein in a suitable sample and comparing the level of expression with a non-cancerous or normal sample.

In some embodiments, an isolated nucleic acid comprises at least 10, 12, 15, 20 or 30 contiguous nucleotides of a sequence selected from the group consisting of the cancer associated polynucleotide sequences disclosed in Table 2 (SEQ ID NOs: 1-55).

In some embodiments, the polynucleotide, or its complement or a fragment thereof, further comprises a detectable label, is attached to a solid support, is prepared at least in part by chemical synthesis, is an antisense fragment, is single stranded, is double stranded or comprises a microarray.

Cancer associated sequences associated with bladder cancer are disclosed in Table 2. These sequences were extracted from hotpop, fold-change and filter analysis KCKC110608.1. Once expression was determined, the gene sequence results were further filtered by considering fold-change in bladder cancer vs. normal bladder; general specificity; secreted or not, level of expression in bladder cancer; and signal to noise ratio. The cancer associated polynucleotide sequences include SEQ ID NOs: 1-55 shown in Table 2. In some embodiments, the polynucleotide sequences may be mRNA sequences selected from: Homo sapiens melanoma antigen family A, 10 (MAGEA10), transcript variant 2; Homo sapiens Down syndrome critical region gene 8 (DSCR8), transcript variant 2; Homo sapiens matrix metallopeptidase 12 (macrophage elastase) (MMP12); Homo sapiens chemokine (C-X-C motif) ligand 9 (CXCL9); Homo sapiens Down syndrome critical region gene 8 (DSCR8), transcript variant 3; Homo sapiens keratin 81 (KRT81); Homo sapiens hypothetical protein LOC729826 (LOC729826); Homo sapiens parathyroid hormone-like hormone (PTHLH), transcript variant 3; Homo sapiens matrix metallopeptidase 11 (stromelysin 3) (MMP11); Homo sapiens S100 calcium binding protein A7 (S100A7); Homo sapiens WNT1 inducible signaling pathway protein 3 (WISP3), transcript variant 1; Homo sapiens chemokine (C-X-C motif) ligand 10 (CXCL10); Homo sapiens neuromedin U (NMU); Homo sapiens guanylate binding protein 5 (GBP5); Homo sapiens topoisomerase (DNA) II alpha 170 kDa (TOP2A); Homo sapiens serpin peptidase inhibitor, clade B (ovalbumin), member 4 (SERPINB4); Homo sapiens granulysin (GNLY), transcript variant 519; Homo sapiens gametocyte specific factor 1 (GTSF1); Homo sapiens peptidase inhibitor 3, skin-derived (SKALP) (PI3); Homo sapiens S100 calcium binding protein A7A (S100A7A); Homo sapiens indoleamine 2,3-dioxygenase 1 (IDO1); Homo sapiens gap junction protein, beta 6 (GJB6); Homo sapiens calmodulin-like 3 (CALML3); Homo sapiens serpin peptidase inhibitor, clade B (ovalbumin), member 3 (SERPINB3); Homo sapiens chemokine (C-X-C motif) ligand 6 (granulocyte chemotactic protein 2) (CXCL6); Homo sapiens olfactomedin 4 (OLFM4); Homo sapiens transcobalamin I (vitamin B12 binding protein, R binder family) (TCN1); Homo sapiens visinin-like 1 (VSNL1); Homo sapiens ubiquitin D (UBD); Homo sapiens absent in melanoma 2 (AIM2); Homo sapiens ATP-binding cassette, sub-family C (CFTR/MRP), member 9 (ABCC9), transcript variant SUR2B; Homo sapiens serpin peptidase inhibitor, clade B (ovalbumin), member 13 (SERPINB13); Homo sapiens indoleamine-pyrrole 2,3 dioxygenase (INDO); Homo sapiens keratin 5 (KRT5); Homo sapiens hypothetical LOC100130897 (LOC100130897); Homo sapiens keratin 14 (epidermolysis bullosa simplex, Dowling-Meara, Koebner) (KRT14); Homo sapiens family with sequence similarity 83, member A (FAM83A), transcript variant 1; Homo sapiens family with sequence similarity 181, member B (FAM181B); RST24587 Athersys RAGE Library Homo sapiens cDNA mRNA sequence; Homo sapiens granzyme B (granzyme 2, cytotoxic T-lymphocyte-associated serine esterase 1) (GZMB); Homo sapiens desmoglein 3 (pemphigus vulgaris antigen) (DSG3); Homo sapiens thymidine phosphorylase (TYMP), transcript variant 3; Homo sapiens keratin 6A (KRT6A); Homo sapiens keratin 6B (KRT6B); a polynucleotide derived therefrom or any combination thereof. In some embodiments, the bladder cancer associated sequences may be DNA sequences encoding the above mRNA or the cancer associated protein or cancer associated polypeptide expressed by the above mRNA. In some embodiments, the cancer associated sequence may be a mutant nucleic acid of the above disclosed sequences. In some embodiments, the cancer associated protein or polypeptide sequence may be selected from SEQ ID NOs: 56-110 or a homolog thereof. In some embodiments, the homolog may have at least about 60%, at least about 65%, at least about 70%, at least about 75%, at least about 80%, at least about 85%, at least about 90%, at least about 95%, at least about 97%, at least about 98%, at least about 99%, at least about 99.5% identity with the disclosed polypeptide sequence.

In some embodiments, a method for diagnosing cancer comprises a) determining the expression of one or more genes comprising a nucleic acid sequence selected from the group consisting of the human genomic and mRNA sequences described in Table 2, in a first sample type (e.g. tissue) of a first individual; and b) comparing said expression of said gene(s) from a second normal sample type from said first individual or a second unaffected individual; wherein a difference in said expression indicates that the first individual has cancer. In some embodiments, the expression is increased as compared to the normal sample. In some embodiments, the expression is decreased as compared to the normal sample.

In some embodiments, the present invention provides methods of diagnosing bladder cancer in a subject, the method comprising: a) determining the expression of one or more genes or gene products or homologs thereof; and b) comparing said expression of the one or more nucleic acid sequences from a second normal sample from said first subject or a second unaffected subject, wherein a difference in said expression indicates that the first subject has cancer, wherein the gene or the gene product is referred to as a gene selected from the group consisting of: MAGEA10, DSCR8, MMP12, CXCL9, DSCR8, KRT81, LOC729826, PTHLH, MMP1, S100A7, WISP3, CXCL10, NMU, GBP5, TOP2A, SERPINB4, GNLY, GTSF1, PI3, S100A7A, IDO1, GJB6, CALML3, SERPINB3, CXCL6, OLFM4, TCN1, VSNL1, UBD, AIM2, ABCC9, SERPINB13, INDO, KRT5, LOC100130897, KRT14, FAM83A, FAM181B, GZMB, DSG3, TYMP, KRT6A, KRT6B, HLA-DRB1, LCN2, KRT4, IFI30, LOC100134370, KIAA1618, S100A8, MMP7, MMP7, SPRR2A, and GJB2.

In some embodiments, the present invention provides methods of detecting bladder cancer in a test sample, comprising: (i) detecting a level of activity of at least one polypeptide that is a gene product; and (ii) comparing the level of activity of the polypeptide in the test sample with a level of activity of polypeptide in a normal sample, wherein an altered level of activity of the polypeptide in the test sample relative to the level of polypeptide activity in the normal sample is indicative of the presence of bladder cancer in the test sample, wherein said gene product is a product of a gene selected from the group consisting of MAGEA10, DSCR8, MMP12, CXCL9, DSCR8, KRT81, LOC729826, PTHLH, MMP11, S100A7, WISP3, CXCL10, NMU, GBP5, TOP2A, SERPINB4, GNLY, GTSF1, PI3, S100A7A, IDO1, GJB6, CALML3, SERPINB3, CXCL6, OLFM4, TCN1, VSNL1, UBD, AIM2, ABCC9, SERPINB13, INDO, KRT5, LOC100130897, KRT14, FAM83A, FAM181B, GZMB, DSG3, TYMP, KRT6A, KRT6B, HLA-DRB1, LCN2, KRT4, IFI30, LOC100134370, KIAA1618, S100A8, MMP7, MMP7, SPRR2A, and GJB2.

In certain embodiments the invention provides a panel of markers associated with bladder cancer comprising nucleic acid sequences, or fragments thereof of the genes: MMP11, MMP12, COL10A1, FCRLB, SFN, KRT6A, S100A2, S100A7 FCRLB, IL1A, KRT16, SLC1A6.

In other embodiments the invention provides a method of detecting bladder cancer in a subject comprising measuring the expression level of the genes MMP11, MMP12, COL10A1, FCRLB, SFN, KRT6A, S100A2, S100A7 IL1A, KRT16, SLC1A6. in a sample, comparing the expression level of the genes MMP11, MMP12, COL10A1, FCRLB, SFN, KRT6A, S100A2, S100A7, IL1A, KRT16, SLC1A6 in a non-cancerous sample such as normal bladder tissue, wherein elevated expression of at least one of the genes MMP11, MMP12, COL10A1, FCRLB, SFN, KRT6A, S100A2, S100A7, IL1A, KRT16, SLC1A6 relative to the non-cancerous sample indicates the subject has cancer. The method may also include comparing the expression level of the genes MMP11, MMP12, COL10A1, FCRLB, SFN, KRT6A, S100A2, S100A7, IL1A, KRT16, SLC1A6 to a known cancerous sample, e.g., a bladder cancer sample, wherein an expression level of at least one of the genes in the subject sample that is at least as high as the known cancer sample indicates the subject has cancer.

In still other embodiments the invention provides a method of detecting bladder cancer in a subject comprising measuring the expression level of the genes MMP11, MMP12, COL10A1, FCRLB, SFN, KRT6A, S100A2, S100A7, IL1A, KRT16, SLC1A6 in sample, comparing the expression level of the genes MMP11, MMP12, COL10A1, FCRLB, SFN, KRT6A, S100A2, S100A7, IL1A, KRT16, SLC1A6 in a non-cancerous sample such as normal bladder tissue, wherein elevated expression of a plurality of the genes MMP11, MMP12, COL10A1, FCRLB, SFN, KRT6A, S100A2, S100A7, IL1A, KRT16, SLC1A6 relative to the non-cancerous sample indicates the subject has cancer. The method may also include comparing the expression level of the genes MMP11, MMP12, COL10A 1, FCRLB, SFN, KRT6A, S100A2, S100A7, IL1A, KRT16, SLC1A6 to a known cancerous sample, e.g., a bladder cancer sample, wherein an expression level of a plurality of the genes in the subject sample that is at least as high as the known cancer sample indicates the subject has cancer.

In still other embodiments the invention provides a method of detecting bladder cancer in a subject comprising measuring the expression level of the genes MMP11, MMP12, COL10A1, FCRLB, SFN, KRT6A, S100A2, S100A7 in sample, comparing the expression level of the genes MMP11, MMP12, COL10A1, FCRLB, SFN, KRT6A, S100A2, S100A7, IL1A, KRT16, SLC1A6 in a non-cancerous sample such as normal bladder tissue, wherein elevated expression of the genes MMP11, MMP12, COL10A1, FCRLB, SFN, KRT6A, S100A2, S100A7, IL1A, KRT16, SLC1A6 relative to the non-cancerous sample indicates the subject has cancer. The method may also include comparing the expression level of the genes MMP11, MMP12, COL10A1, FCRLB, SFN, KRT6A, S100A2, S100A7, IL1A, KRT16, SLC1A6 to a known cancerous sample, e.g., a bladder cancer sample, wherein an expression level of the genes in the subject sample that is at least as high as the known cancer sample indicates the subject has cancer.

### Cancer Associated Proteins and Cancer Detection

Cancer associated sequences may also include proteins or peptides encoded by the nucleic acid sequences described above. A list of proteins or peptides associated with bladder cancer is provided in Table 3. The amino acid sequences encoding these proteins or peptides are provided in Table 6.

In some embodiments, the cancer associated sequence comprises a sequence of a naturally occurring protein. In some embodiments, the cancer associate sequence does not comprise a naturally occurring sequence. Thus in some embodiments the cancer associated sequence may encode a mutant protein or a fragment of a naturally occurring protein.

In some embodiments, the invention provides a method for detecting a cancer associated sequence with the expression of a polypeptide in a test sample, comprising detecting a level of expression of at least one polypeptide selected from the group consisting of SEQ ID NOs: 56-110 shown in Table 3, or a fragment thereof. In some embodiments, the method comprises comparing the level of expression of the polypeptide in the test sample with a level of expression of polypeptide in a normal sample, wherein an altered level of expression of the polypeptide, e.g. elevated expression, in the test sample relative to the level of polypeptide expression in the normal sample is indicative of the presence of cancer in the test sample. In some embodiments, the polypeptide expression is compared to a cancer sample, wherein the level of expression is at least the same as the cancer is indicative of the presence of cancer in the test sample. In some embodiments, the sample is a cell sample.

In some embodiments, the invention provides an isolated polypeptide, encoded within an open reading frame of a cancer associated sequence selected from the polynucleotide sequences of SEQ ID NOs: 56-110 shown in Table 3, or its complement. In some embodiments, the invention provides an isolated polypeptide, wherein said polypeptide comprises the amino acid sequence encoded by a polynucleotide selected from the group consisting of SEQ ID NOs: 1-55. In some embodiments, the invention provides an isolated polypeptide, wherein said polypeptide comprises the amino acid sequence encoded by a polypeptide selected from the group consisting of SEQ ID NOs: 56-110, shown in Table 3.

In some embodiments, the invention further provides an isolated polypeptide, comprising the amino acid sequence of an epitope of the amino acid sequence of a cancer associated polypeptide selected from the group consisting of SEQ ID. NOs: 56-110 shown in Table 3, wherein the polypeptide or fragment thereof may be attached to a solid support. In some embodiments the invention provides an isolated antibody (monoclonal or polyclonal) or antigen binding fragment thereof, that binds to such a polypeptide. The isolated antibody or antigen binding fragment thereof may be attached to a solid support, or further comprises a detectable label.

In some embodiments, the invention provides a method for detecting cancer by detecting the presence of an antibody in a test serum sample. In some embodiments, the antibody recognizes a polypeptide or an epitope thereof disclosed herein. In some embodiments, the antibody recognizes a polypeptide or epitope thereof encoded by a nucleic acid sequence disclosed herein. In some embodiments, the method comprises detecting a level of an antibody against an antigenic polypeptide selected from the group consisting of SEQ ID NOs: 56-110 shown in Table 3, or antigenic fragment thereof. In some embodiments, the method comprises comparing the level of the antibody in the test sample with a level of the antibody in the control sample, wherein an altered level of antibody in said test sample relative to the level of antibody in the control sample is indicative of the presence of cancer in the test sample. In some embodiments, the control sample is a sample derived from a normal cell or non-cancerous sample. In some embodiments, the control is derived from a cancer sample, and, therefore, in some embodiments, the method comprises comparing the levels of binding and/or the amount of antibody in the sample, wherein when the levels or amount are the same as the cancer control sample is indicative of the presence of cancer in the test sample.

In some embodiments, the invention also provides a method for detecting presence or absence of cancer cells in a subject. In some embodiments, the method comprises contacting one or more cells from the subject with an antibody as described herein. In some embodiments, the method comprises detecting a complex of a cancer associated protein (CAP) and the antibody, wherein detection of the complex indicates with the presence of cancer cells in the subject.

In still other embodiments the invention provides a method of detecting bladder cancer in a subject comprising measuring the protein expression level of the protein encoded for by the genes MMP11, MMP12, COL10A1, FCRLB, SFN, KRT6A, S100A2, S100A7, IL1A, KRT16, SLC1A6 in sample, comparing the expression level of the protein encoded for by the genes MMP11, MMP12, COL10A1, FCRLB, SFN, KRT6A, S100A2, S100A7, IL1A, KRT16, SLC1A6 in a non-cancerous sample such as normal bladder tissue, wherein elevated expression of at least one of the protein encoded for by the genes MMP11, MMP12, COL10A1, FCRLB, SFN, KRT6A, S100A2, S100A7, IL1A, KRT16, SLC1A6 relative to the non-cancerous sample indicates the subject has cancer. The method may also include comparing the expression level of the protein encoded for by the genes MMP11, MMP12, COL10A1, FCRLB, SFN, KRT6A, S100A2, S100A7, IL1A, KRT16, SLC1A6 to a known cancerous sample, e.g., a bladder cancer sample, wherein an expression level of at least one of the proteins encoded for by the genes in the subject sample that is at least as high as the known cancer sample indicates the subject has cancer.

In still other embodiments the invention provides a method of detecting bladder cancer in a subject comprising measuring the protein expression level of the protein encoded for by the genes MMP11, MMP12, COL10A1, FCRLB, SFN, KRT6A, S100A2, S100A7, IL1A, KRT16, SLC1A6 in sample, comparing the expression level of the protein encoded for by the genes MMP11, MMP12, COL10A1, FCRLB, SFN, KRT6A, S100A2, S100A7, IL1A, KRT16, SLC1A6 in a non-cancerous sample such as normal bladder tissue, wherein elevated expression of a plurality of the protein encoded for by the genes MMP11, MMP12, COL10A1, FCRLB, SFN, KRT6A, S100A2, S100A7, IL1A, KRT16, SLC1A6 relative to the non-cancerous sample indicates the subject has cancer. The method may also include comparing the expression level of the protein encoded for by the genes MMP11, MMP12, COL10A1, FCRLB, SFN, KRT6A, S100A2, S100A7, IL1A, KRT16, SLC1A6 to a known cancerous sample, e.g., a bladder cancer sample, wherein an expression level of a plurality of the proteins encoded for by the genes in the subject sample that is at least as high as the known cancer sample indicates the subject has cancer.

In still other embodiments the invention provides a method of detecting bladder cancer in a subject comprising measuring the protein expression level of the protein encoded for by the genes MMP11, MMP12, COL10A1, FCRLB, SFN, KRT6A, S100A2, S100A7, IL1A, KRT16, SLC1A6 in sample, comparing the expression level of the protein encoded for by the genes MMP11, MMP12, COL10A1, FCRLB, SFN, KRT6A, S100A2, S100A7, IL1A, KRT16, SLC1A6 in a non-cancerous sample such as normal bladder tissue, wherein elevated expression of the proteins encoded for by the genes MMP11, MMP12, COL10A1, FCRLB, SFN, KRT6A, S100A2, S100A7, IL1A, KRT16, SLC1A6 relative to the non-cancerous sample indicates the subject has cancer. The method may also include comparing the expression level of the protein encoded for by the genes MMP11, MMP12, COL10A1, FCRLB, SFN, KRT6A, S100A2, S100A7, IL1A, KRT16, SLC1A6 to a known cancerous sample, e.g., a bladder cancer sample, wherein an expression level of the proteins encoded for by the genes in the subject sample that is at least as high as the known cancer sample indicates the subject has cancer.

### Immune Response to Cancer Associated Proteins

Some embodiments are directed to the use of cancer associated polypeptides and polynucleotides encoding a cancer associated sequence, a fragment thereof, or a mutant thereof, and antigen presenting cells (such as, without limitation, dendritic cells), to elicit an immune response against cells expressing a cancer-associated polypeptide sequence, such as, without limitation, cancer cells, (in a subject, or in vitro) such as bladder cancer cells. In some embodiments, the method of eliciting an immune response against cells expressing a cancer associated sequence comprises (1) isolating a hematopoietic stem cell, (2) genetically modifying the cell to express a cancer associated sequence, (3) differentiating the cell into DCs; and (4) administering the DCs to the subject (e.g., human patient). In some embodiments, antigen presenting cells (APCs) may used to activate T lymphocytes in vivo or ex vivo, to elicit an immune response against cells expressing a cancer associated sequence. APCs are highly specialized cells and may include, without limitation, macrophages, monocytes, and dendritic cells (DCs). APCs may process antigens and display their peptide fragments on the cell surface together with molecules required for lymphocyte activation. In some embodiments, the APCs may be dendritic cells. DCs may be classified into subgroups, including, e.g., follicular dendritic cells, Langerhans dendritic cells, and epidermal dendritic cells. In some embodiments, dendritic cell precursor cells are isolated for transduction with a cancer associated sequence, and induced to differentiate into dendritic cells. The genetically modified DCs express the cancer associated sequence, and may display peptide fragments on the cell surface.

In some embodiments, the method of eliciting an immune response includes (1) isolating DCs (or isolation and differentiation of DC precursor cells), (2) pulsing the cells with a cancer associated sequence, and; (3) administering the DCs to the subject. These approaches are discussed in greater detail, infra. In some embodiments, the pulsed or expressing DCs may be used to activate T lymphocytes ex vivo. These general techniques and variations thereof may be within the skill of those in the art (see, e.g., WO97/29182; WO 97/04802; WO 97/22349; WO 96/23060; WO 98/01538; Hsu et al., 1996, Nature Med. 2:52-58), and that still other variations may be discovered in the future.

In some embodiments, the cancer associated sequence is contacted with a subject to stimulate an immune response. In some embodiments, the immune response is a therapeutic immune response. In some embodiments, the immune response is a prophylactic immune response. For example, the cancer associated sequence can be contacted with a subject under conditions effective to stimulate an immune response. The cancer associated sequence can be administered as, for example, a DNA molecule (e.g. DNA vaccine), RNA molecule, or polypeptide, or any combination thereof. Administering sequence to stimulate an immune responses are known, but the identity of which sequences to use was not known prior to the present disclosure. Any sequence or combination of sequences disclosed herein or a homolog thereof can be administered to a subject to stimulate an immune response.

In some embodiments, the cancer associated sequence comprises a sequence of a naturally occurring protein. In some embodiments, the cancer associate sequence does not comprise a naturally occurring sequence. As already noted, fragments of naturally occurring proteins may be used; in addition, the expressed polypeptide may comprise mutations such as deletions, insertions, or amino acid substitutions when compared to a naturally occurring polypeptide, so long as at least one peptide epitope can be processed by the DC and presented on a MHC class I or II surface molecule. In some embodiments, it may be desirable to use sequences other than “wild type,” in order to, for example, increase antigenicity of the peptide or to increase peptide expression levels. In some embodiments, the introduced cancer associated sequences may encode variants such as polymorphic variants (e.g., a variant expressed by a particular human patient) or variants characteristic of a particular cancer (e.g., a cancer in a particular subject).

In some embodiments, a cancer associated expression sequence may be introduced (transduced) into DCs or stem cells in any of a variety of standard methods, including transfection, recombinant vaccinia viruses, adeno-associated viruses (AAVs), retroviruses, etc.

In some embodiments, the transformed DCs of the invention may be introduced into the subject (e.g., without limitation, a human patient) where the DCs may induce an immune response. Typically, the immune response includes a cytotoxic T-lymphocyte (CTL) response against target cells bearing antigenic peptides (e.g., in a MHC class I/peptide complex). These target cells are typically cancer cells.

In some embodiments, when the DCs of the invention are to be administered to a subject, they may preferably isolated from, or derived from precursor cells from, that subject (i.e., the DCs may administered to an autologous subject). However, the cells may be infused into HLA-matched allogeneic, or HLA-mismatched allogeneic subject. In the latter case, immunosuppressive drugs may be administered to the subject.

In some embodiments, the cells tray be administered in any suitable manner. In some embodiments, the cell may be administered with a pharmaceutically acceptable carrier (e.g., saline). In some embodiments, the cells may be administered through intravenous, intra-articular, intramuscular, intradermal, intraperitoneal, or subcutaneous routes. Administration (i.e., immunization) may be repeated at time intervals. Infusions of DC may be combined with administration of cytokines that act to maintain DC number and activity (e.g., GM-CSF, IL-12).

In some embodiments, the dose administered to a subject may be a dose sufficient to induce an immune response as detected by assays which measure T cell proliferation, T lymphocyte cytotoxicity, and/or effect a beneficial therapeutic response in the patient over time, e.g., to inhibit growth of cancer cells or result in reduction in the number of cancer cells or the size of a tumor.

In some embodiments, DCs are obtained (either from a patient or by in vitro differentiation of precursor cells) and pulsed with antigenic peptides having a cancer associated sequence. The pulsing results in the presentation of peptides onto the surface MHC molecules of the cells. The peptide/MHC complexes displayed on the cell surface may be capable of inducing a MHC-restricted cytotoxic T-lymphocyte response against target cells expressing cancer associated polypeptides (e.g., without limitations, cancer cells).

In some embodiments, cancer associated sequences used for pulsing may have at least about 6 or 8 amino acids and fewer than about 30 amino acids or fewer than about 50 amino acid residues in length. In some embodiments, an immunogenic peptide sequence may have from about 8 to about 12 amino acids. In some embodiments, a mixture of human protein fragments may be used; alternatively a particular peptide of defined sequence may be used. The peptide antigens may be produced by de novo peptide synthesis, enzymatic digestion of purified or recombinant human peptides, by purification of the peptide sequence from a natural source (e.g., a subject or tumor cells from a subject), or expression of a recombinant polynucleotide encoding a human peptide fragment.

In some embodiments, the amount of peptide used for pulsing DC may depend on the nature, size and purity of the peptide or polypeptide. In some embodiments, an amount of from about 0.05 ug/ml to about 1 mg/ml, from about 0.05 ug/ml to about 500 ug/ml, from about 0.05 ug/ml to about 250 ug/ml, from about 0.5 ug/ml to about 1 mg/ml, from about 0.5 ug/ml to about 500 ug/ml, from about 0.5 ug/ml to about 250 ug/ml, or from about 1 ug/ml to about 100 ug/ml of peptide may be used. After adding the peptide antigen(s) to the cultured DC, the cells may then be allowed sufficient time to take up and process the antigen and express antigen peptides on the cell surface in association with either class I or class II MHC. In some embodiments, the time to take up and process the antigen may be about 18 to about 30 hours, about 20 to about 30 hours, or about 24 hours.

Numerous examples of systems and methods for predicting peptide binding motifs for different MHC Class I and II molecules have been described. Such prediction could be used for predicting peptide motifs that will bind to the desired MHC Class I or II molecules. Examples of such methods, systems, and databases that those of ordinary skill in the art might consult for such purpose include NIH's Center for Information Technology and Peptide Binding Motifs for MHC Class I and II Molecules; William E. Biddison, Roland Martin, Current Protocols in Immunology, Unit 1I (DOI: 10.1002/0471142735.ima01 is 36; Online Posting Date: May, 2001), which provides an overview of the use of peptide-binding motifs to predict interaction with a specific MHC class I or II allele, and gives examples for the use of MHC binding motifs to predict T-cell recognition.

Table I provides an exemplary result for a HLA peptide motif search at the NIH Center for Information Technology website, BioInformatics and Molecular Analysis Section. The fill length MAGEA10 peptide sequence (SEQ ID NO: 56 as shown in Table 3 and 5) was used as the search query.

In some embodiments, the present invention provides methods of eliciting an immune response against cells expressing a cancer associated sequence comprising contacting a subject with a a cancer associated sequence under conditions effective to elicit an immune response in the subject, wherein said cancer associated sequence comprises a sequence or fragment thereof a gene selected from the group consisting of MAGEA10, DSCR8, MMP12, CXCL9, DSCR8, KRT81, LOC729826, PTHLH, MMP11, S100A7, WISP3, CXCL10, NMU, GBP5, TOP2A, SERPINB4, GNLY, GTSF1, PI3, S100A7A, IDO1, GJB6, CALML3, SERPINB3, CXCL6, OLFM4, TCN1, VSNL1, UBD, AIM2, ABCC9, SERPINB13, INDO, KRT5, LOC100130897, KRT14, FAM83A, FAM181B, GZMB, DSG3, TYMP, KRT6A, KRT6B, HLA-DRB1, LCN2, KRT4, IFI30, LOC100134370, KIAA1618, S100A8, MMP7, MMP7, SPRR2A, and GJB2.

### Immunotherapy

In some embodiments, implementation of an immunotherapy strategy for treating, reducing the symptoms of, or preventing cancer or neoplasms, (e.g., a vaccine) may be achieved using many different techniques available to the skilled artisan.

Immunotherapy, or the use of antibodies for therapeutic purposes has been used in recent years to treat cancer. Passive immunotherapy involves the use of monoclonal antibodies in cancer treatments. See for example, Cancer: Principles and Practice of Oncology, 6th Edition (2001) Chapt. 20 pp. 495-508. Inherent therapeutic biological activity of these antibodies include direct inhibition of tumor cell growth or survival, and the ability to recruit the natural cell killing activity of the body's immune system. These agents may be administered alone or in conjunction with radiation or chemotherapeutic agents. Alternatively, antibodies may be used to make antibody conjugates where the antibody is linked to a toxic agent and directs that agent to the tumor by specifically binding to the tumor.

Some embodiments also provide for antigens (cancer-associated polypeptides) associated with a variety of cancers, including bladder cancer, as targets for diagnostic and/or therapeutic antibodies. These antigens may also be useful for drug discovery (e.g., small molecules) and for further characterization of cellular regulation, growth, and differentiation.

TABLE 1
User Parameters and Scoring Information

| | |
|---|---|
| method selected to limit number of | explicit |
| results | number |
| number of results requested | 20 |
| HLA molecule type selected | A_0201 |
| length selected for subsequences to be | 9 |
| scored | |
| echoing mode selected for input sequence | Y |
| echoing format | numbered |
| | lines |
| length of user's input peptide sequence | 369 |
| number of subsequence scores calculated | 361 |
| number of top scoring subsequences | 20 |
| reported back in scoring output table | |

Scoring Results

| | | | Score (Estimate |
|---|---|---|---|
| | | | of Half Time of |
| | | | Disassociation of |
| | Start | Subsequence | a Molecule Containing |
| Rank | Position | Residue Listing | This Subsequence) |
| 1 | 310 | SLLKFLAKV | 2249.173 |
| 2 | 183 | MLLVFGIDV | 1662.432 |
| 3 | 137 | KVTDLVQFT | 339.313 |
| 4 | 254 | GLYDGMEHL | 315.870 |
| 5 | 228 | ILILSIIFI | 224.357 |
| 6 | 296 | FLWGPRAHA | 189.678 |
| 7 | 245 | VIWEALNHH | 90.891 |
| 8 | 308 | KMSLLKFLA | 72.836 |
| 9 | 166 | KMYEDHFPL | 37.140 |
| 10 | 201 | FVLVTSLGL | 31.814 |
| 11 | 174 | LLFSEASEC | 31.249 |
| 12 | 213 | GMLSDVQSM | 30.534 |
| 13 | 226 | ILILLILSII | 16.725 |
| 14 | 225 | GILILILSI | 12.208 |
| 15 | 251 | NKMGLYCGH | 9.758 |
| 16 | 88 | QIACSSPSV | 9.563 |
| 17 | 66 | LIFSTPEEV | 7.966 |
| 18 | 220 | SHPKTGILI | 7.535 |
| 19 | 233 | IIFIEGYCT | 6.445 |
| 20 | 247 | WEALNMMGL | 4.395 |

Rank 1-20 are assigned SEQ ID NOS: 198-217 respectively

TABLE 2

| SEQ ID | | | mRNA |
|---|---|---|---|
| NO: | Symbol | Definition | Sequence |
| 1 | MAGEA10 | Homo sapiens melanoma antigen family A, 10 (MAGEA10), | NM_021048.3 |
| | | transcript variant 2, mRNA. | |
| 2 | DSCR8 | Homo sapiens Down syndrome critical region gene 8 | NM_203428.1 |
| | | (DSCR8), transcript variant 2, mRNA. | |
| 3 | MMP12 | Homo sapiens matrix metallopeptidase 12 (macrophage | NM_002426.2 |
| | | elastase) (MMP12), mRNA. | |
| 4 | CXCL9 | Homo sapiens chemokine (C-X-C motif) ligand 9 (CXCL9), | NM_002416.1 |
| | | mRNA. | |
| 5 | DSCR8 | Homo sapiens Down syndrome critical region gene 8 | NM_203429.1 |
| | | (DSCR8), transcript variant 3, mRNA. | |
| 6 | KRT81 | Homo sapiens keratin 81 (KRT81), mRNA. | NM_002281.2 |
| 7 | LOC729826 | PREDICTED: Homo sapiens hypothetical protein | XM_001131447.1 |
| | | LOC729826 (LOC729826), mRNA. | |
| 8 | PTHLH | Homo sapiens parathyroid hormone-like hormone (PTHLH), | NM_198964.1 |
| | | transcript variant 3, mRNA. | |
| 9 | MMP11 | Homo sapiens matrix metallopeptidase 11 (stromelysin 3) | NM_005940.3 |
| | | (MMP11), mRNA. | |
| 10 | S100A7 | Homo sapiens S100 calcium binding protein A7 (S100A7), | NM_002963.3 |
| | | mRNA. | |
| 11 | WISP3 | Homo sapiens WNT1 inducible signaling pathway protein 3 | NM_003880.2 |
| | | (WISP3), transcript variant 1, mRNA. | |
| 12 | CXCL10 | Homo sapiens chemokine (C-X-C motif) ligand 10 | NM_001565.2 |
| | | (CXCL10), mRNA. | |
| 13 | NMU | Homo sapiens neuromedin U (NMU), mRNA. | NM_006681.1 |
| 14 | GBP5 | Homo sapiens guanylate binding protein 5 (GBP5), mRNA. | NM_052942.2 |
| 15 | TOP2A | Homo sapiens topoisomerase (DNA) II alpha 170 kDa | NM_001067.2 |
| | | (TOP2A), mRNA. | |
| 16 | SERPINB4 | Homo sapiens serpin peptidase inhibitor, clade B | NM_002974.2 |
| | | (ovalbumin), member 4 (SERPINB4), mRNA. | |
| 17 | GNLY | Homo sapiens granulysin (GNLY), transcript variant 519, | NM_012483.1 |
| | | mRNA. | |
| 18 | GTSF1 | Homo sapiens gametocyte specific factor 1 (GTSF1), | NM_144594.1 |
| | | mRNA. | |
| 19 | PI3 | Homo sapiens peptidase inhibitor 3, skin-derived (SKALP) | NM_002638.2 |
| | | (PI3), mRNA. | |
| 20 | S100A7A | Homo sapiens S100 calcium binding protein A7A | NM_176823.3 |
| | | (S100A7A), mRNA. | |
| 21 | IDO1 | Homo sapiens indoleamine 2,3-dioxygenase 1 (IDO1), | NM_002164.4 |
| | | mRNA. | |
| 22 | GJB6 | Homo sapiens gap junction protein, beta 6 (GJB6), mRNA. | NM_006783.2 |
| 23 | CALML3 | Homo sapiens calmodulin-like 3 (CALML3), mRNA. | NM_005185.2 |
| 24 | SERPINB3 | Homo sapiens serpin peptidase inhibitor, clade B | NM_006919.1 |
| | | (ovalbumin), member 3 (SERPINB3), mRNA. | |
| 25 | CXCL6 | Homo sapiens chemokine (C-X-C motif) ligand 6 | NM_002993.2 |
| | | (granulocyte chemotactic protein 2) (CXCL6), mRNA. | |
| 26 | OLFM4 | Homo sapiens olfactomedin 4 (OLFM4), mRNA. | NM_006418.3 |
| 27 | TCN1 | Homo sapiens transcobalamin I (vitamin B12 binding | NM_001062.3 |
| | | protein, R binder family) (TCN1), mRNA. | |
| 28 | VSNL1 | Homo sapiens visinin-like 1 (VSNL1), mRNA. | NM_003385.4 |
| 29 | UBD | Homo sapiens ubiquitin D (UBD), mRNA. | NM_006398.2 |
| 30 | AIM2 | Homo sapiens absent in melanoma 2 (AIM2), mRNA. | NM_004833.1 |
| 31 | ABCC9 | Homo sapiens ATP-binding cassette, sub-family C | NM_020297.1 |
| | | (CFTR/MRP), member 9 (ABCC9), transcript variant | |
| | | SUR2B, mRNA. | |
| 32 | SERPINB13 | Homo sapiens serpin peptidase inhibitor, clade B | NM_012397.2 |
| | | (ovalbumin), member 13 (SERPINB13), mRNA. | |
| 33 | INDO | Homo sapiens indoleamine-pyrrole 2,3 dioxygenase (INDO), | NM_002164.3 |
| | | mRNA. | |
| 34 | KRT5 | Homo sapiens keratin 5 (KRT5), mRNA. | NM_000424.3 |
| 35 | LOC100130897 | PREDICTED: Homo sapiens hypothetical LOC100130897 | XM_001718498.1 |
| | | (LOC100130897), mRNA. | |
| 36 | KRT14 | Homo sapiens keratin 14 (epidermolysis bullosa simplex, | NM_000526.3 |
| | | Dowling-Meara, Koebner) (KRT14), mRNA. | |
| 37 | FAM83A | Homo sapiens family with sequence similarity 83, member | NM_032899.4 |
| | | A (FAM83A), transcript variant 1, mRNA. | |
| 38 | FAM181B | Homo sapiens family with sequence similarity 181, member | NM_175885.3 |
| | | B (FAM181B), mRNA. | |
| 39 | | RST24587 Athersys RAGE Library Homo sapiens cDNA, | BG205162 |
| | | mRNA sequence | |
| 40 | GZMB | Homo sapiens granzyme B (granzyme 2, cytotoxic T- | NM_004131.3 |
| | | lymphocyte-associated serine esterase 1) (GZMB), mRNA. | |
| 41 | DSG3 | Homo sapiens desmoglein 3 (pemphigus vulgaris antigen) | NM_001944.2 |
| | | (DSG3), mRNA. | |
| 42 | TYMP | Homo sapiens thymidine phosphorylase (TYMP), transcript | NM_001113756.1 |
| | | variant 3, mRNA. | |
| 43 | KRT6A | Homo sapiens keratin 6A (KRT6A), mRNA. | NM_005554.3 |
| 44 | KRT6B | Homo sapiens keratin 6B (KRT6B), mRNA. | NM_005555.3 |
| 45 | HLA-DRB1 | Homo sapiens major histocompatibility complex, class II, | NM_002124.1 |
| | | DR beta 1 (HLA-DRB1), mRNA. | |
| 46 | LCN2 | Homo sapiens lipocalin 2 (LCN2), mRNA. | NM_005564.3 |
| 47 | KRT4 | Homo sapiens keratin 4 (KRT4), mRNA. | NM_002272.2 |
| 48 | IFI30 | Homo sapiens interferon, gamma-inducible protein 30 | NM_006332.3 |
| | | (IFI30), mRNA. | |
| 49 | LOC100134370 | PREDICTED: Homo sapiens hypothetical protein | XM_001713687.1 |
| | | LOC100134370 (LOC100134370), mRNA. | |
| 50 | KIAA1618 | Homo sapiens KIAA1618 (KIAA1618), mRNA. | NM_020954.2 |
| 51 | S100A8 | Homo sapiens S100 calcium binding protein A8 (S100A8), | NM_002964.3 |
| | | mRNA. | |
| 52 | MMP7 | Homo sapiens matrix metallopeptidase 7 (matrilysin, | NM_002423.3 |
| | | uterine) (MMP7), mRNA. | |
| 53 | MMP7 | Homo sapiens matrix metallopeptidase 7 (matrilysin, | NM_002423.3 |
| | | uterine) (MMP7), mRNA. | |
| 54 | SPRR2A | Homo sapiens small proline-rich protein 2A (SPRR2A), | NM_005988.2 |
| | | mRNA. | |
| 55 | GJB2 | Homo sapiens gap junction protein, beta 2, 26 kDa (GJB2), | NM_004004.4 |
| | | mRNA. | |

TABLE 3

| SEQ ID | | | Peptide |
|---|---|---|---|
| NO: | Symbol | Definition | Sequence |
| 56 | MAGEA10 | Homo sapiens melanoma antigen family A, 10 (MAGEA10), transcript | NP_066386.1 |
| | | variant 2 | |
| 57 | DSCR8 | Homo sapiens Down syndrome critical region gene 8 (DSCR8), transcript | NP_982252.1 |
| | | variant 2 | |
| 58 | MMP12 | Homo sapiens matrix metallopeptidase 12 (macrophage elastase) (MMP12) | NP_002417.2 |
| 59 | CXCL9 | Homo sapiens chemokine (C-X-C motif) ligand 9 (CXCL9) | NP_002407.1 |
| 60 | DSCR8 | Homo sapiens Down syndrome critical region gene 8 (DSCR8), transcript | NP_982253.1 |
| | | variant 3. | |
| 61 | KRT81 | Homo sapiens keratin 81 (KRT81). | NP_002272.1 |
| 62 | LOC729826 | PREDICTED: Homo sapiens hypothetical protein LOC729826 | XP_001131447.1 |
| | | (LOC729826). | |
| 63 | PTHLH | Homo sapiens parathyroid hormone-like hormone (PTHLH), transcript | NP_945315.1 |
| | | variant 3 | |
| 64 | MMP11 | Homo sapiens matrix metallopeptidase 11 (stromelysin 3) (MMP11) | NP_005931.2 |
| 65 | S100A7 | Homo sapiens S100 calcium binding protein A7 (S100A7) | NP_002954.2 |
| 66 | WISP3 | Homo sapiens WNT1 inducible signaling pathway protein 3 (WISP3), | NP_003871.1 |
| | | transcript variant 1 | |
| 67 | CXCL10 | Homo sapiens chemokine (C-X-C motif) ligand 10 (CXCL10) | NP_001556.2 |
| 68 | NMU | Homo sapiens neuromedin U (NMU) | NP_006672.1 |
| 69 | GBP5 | Homo sapiens guanylate binding protein 5 (GBP5) | NP_443174.1 |
| 70 | TOP2A | Homo sapiens topoisomerase (DNA) II alpha 170 kDa (TOP2A) | |
| 71 | SERPINB4 | Homo sapiens serpin peptidase inhibitor, clade B (ovalbumin), member 4 | NP_002965.1 |
| | | (SERPINB4) | |
| 72 | GNLY | Homo sapiens granulysin (GNLY), transcript variant 519 | NP_036615.1 |
| 73 | GTSF1 | Homo sapiens gametocyte specific factor 1 (GTSF1) | NP_653195.1 |
| 74 | PI3 | Homo sapiens peptidase inhibitor 3, skin-derived (SKALP) (PI3) | NP_002629.1 |
| 75 | S100A7A | Homo sapiens S100 calcium binding protein A7A (S100A7A) | NP_789793.1 |
| 76 | IDO1 | Homo sapiens indoleamine 2,3-dioxygenase 1 (IDO1) | NP_002155.1 |
| 77 | GJB6 | Homo sapiens gap junction protein, beta 6 (GJB6) | NP_006774.2 |
| 78 | CALML3 | Homo sapiens calmodulin-like 3 (CALML3) | NP_005176.1 |
| 79 | SERPINB3 | Homo sapiens serpin peptidase inhibitor, clade B (ovalbumin), member 3 | NP_008850.1 |
| | | (SERPINB3) | |
| 80 | CXCL6 | Homo sapiens chemokine (C-X-C motif) ligand 6 (granulocyte chemotactic | NP_002984.1 |
| | | protein 2) (CXCL6) | |
| 81 | OLFM4 | Homo sapiens olfactomedin 4 (OLFM4) | NP_006409.3 |
| 82 | TCN1 | Homo sapiens transcobalamin I (vitamin B12 binding protein, R binder | NP_001053.2 |
| | | family) (TCN1) | |
| 83 | VSNL1 | Homo sapiens visinin-like 1 (VSNL1) | NP_003376.2 |
| 84 | UBD | Homo sapiens ubiquitin D (UBD) | NP_006389.1 |
| 85 | AIM2 | Homo sapiens absent in melanoma 2 (AIM2) | NP_004824.1 |
| 86 | ABCC9 | Homo sapiens ATP-binding cassette, sub-family C (CFTR/MRP), member | NP_064693.1 |
| | | 9 (ABCC9), transcript variant SUR2B | |
| 87 | SERPINB13 | Homo sapiens serpin peptidase inhibitor, clade B (ovalbumin), member 13 | NP_036529.1 |
| | | (SERPINB13) | |
| 88 | INDO | Homo sapiens indoleamine-pyrrole 2,3 dioxygenase (INDO) | NP_002155.1 |
| 89 | KRT5 | Homo sapiens keratin 5 (KRT5) | NP_000415.2 |
| 90 | LOC100130897 | PREDICTED: Homo sapiens hypothetical LOC100130897 | XP_001718550.1 |
| | | (LOC100130897) | |
| 91 | KRT14 | Homo sapiens keratin 14 (epidermolysis bullosa simplex, Dowling-Meara, | NP_000517.2 |
| | | Koebner) (KRT14) | |
| 92 | FAM83A | Homo sapiens family with sequence similarity 83, member A (FAM83A), | NP_116288.2 |
| | | transcript variant 1 | |
| 93 | FAM181B | Homo sapiens family with sequence similarity 181, member B (FAM181B) | NP_787081.2 |
| 94 | | RST24587 Athersys RAGE Library Homo sapiens cDNA | |
| 95 | GZMB | Homo sapiens granzyme B (granzyme 2, cytotoxic T-lymphocyte- | NP_004122.1 |
| | | associated serine esterase 1) (GZMB) | |
| 96 | DSG3 | Homo sapiens desmoglein 3 (pemphigus vulgaris antigen) (DSG3) | NP_001935.2 |
| 97 | TYMP | Homo sapiens thymidine phosphorylase (TYMP), transcript variant 3 | NP_001107228.1 |
| 98 | KRT6A | Homo sapiens keratin 6A (KRT6A) | NP_005545.1 |
| 99 | KRT6B | Homo sapiens keratin 6B (KRT6B) | NP_005546.2 |
| 100 | HLA-DRB1 | Homo sapiens major histocompatibility complex, class II, DR beta 1 (HLA- | NP_002115.1 |
| | | DRB1) | |
| 101 | LCN2 | Homo sapiens lipocalin 2 (LCN2) | NP_005555.2 |
| 102 | KRT4 | Homo sapiens keratin 4 (KRT4) | NP_002263.2 |
| 103 | IFI30 | Homo sapiens interferon, gamma-inducible protein 30 (IFI30) | NP_006323.2 |
| 104 | LOC100134370 | PREDICTED: Homo sapiens hypothetical protein LOC100134370 | XP_001713739.1 |
| | | (LOC100134370) | |
| 105 | KIAA1618 | Homo sapiens KIAA1618 (KIAA1618) | NP_066005.2 |
| 106 | S100A8 | Homo sapiens S100 calcium binding protein A8 (S100A8) | NP_002955.2 |
| 107 | MMP7 | Homo sapiens matrix metallopeptidase 7 (matrilysin, uterine) (MMP7) | NP_002414.1 |
| 108 | MMP7 | Homo sapiens matrix metallopeptidase 7 (matrilysin, uterine) (MMP7) | NP_002414.1 |
| 109 | SPRR2A | Homo sapiens small proline-rich protein 2A (SPRR2A) | NP_005979.1 |
| 110 | GJB2 | Homo sapiens gap junction protein, beta 2, 26 kDa (GJB2) | NP_003995.2 |

TABLE 4

| SEQ ID | | |
|---|---|---|
| No. | Probe Sequence | Symbol |
| 111 | GCCATGGCCAGTGCAAGTTCTAGCGCTACAGGTAGCTTCTCCTACCCTGA | MAGEA10 |
| 112 | TCCCACTTGGCAGGGGCCGTCTTGTCCACTCGTTTCTGTAAACATGGGTG | DSCR8 |
| 113 | TCTATTTGAAGCATGCTCTGTAAGTTGCTTCCTAACATCCTTGGACTGAG | MMP12 |
| 114 | TGATTGGTGCCCAGTTAGCCTCTGCAGGATGTGGAAACCTCCTTCCAGGG | CXCL9 |
| 115 | GAAGGCTGGCTCATACATTTTCCCAGACAGGAATTTGGCTGCCAACAGGG | DSCR8 |
| 116 | CAGTGGGAAAGGCCACCCTAGAAAGAAGTCCGCTGGCACCCATAGGAAGG | KRT81 |
| 117 | CCTGCAGACACCGGAGGCCTCTGCTGTGGCTGCCCACTGGCTGTGCTCAG | LOC729826 |
| 118 | TGGTTAGACTCTGGAGTGACTGGGAGTGGGCTAGAAGGGGACCACCTGTC | PTHLH |
| 119 | CAGGTCTTGGTAGGTGCCTGCATCTGTCTGCCTTCTGGCTGACAATCCTG | MMP11 |
| 120 | GCTGAGAGGTCCATAATAGGCATGATCGACATGTTTCACAAATACACCAG | S100A7 |
| 121 | GCTGTGGATTACATCTTGTGTGTGTCAGAGAAACTGCAGAGAACCTGGAG | WISP3 |
| 122 | GACTTCCACTGCCATCCTCCCAAGGGGCCCAAATTCTTTCAGTGGCTACC | CXCL10 |
| 123 | GCTGCAGCTCGTTCCTCACCTGCATGAGAGAAGAATGAAGAGATTCAGAG | NMU |
| 124 | GCAGGAACAACAGATGCAGGAACAGGCTGCACAGCTCAGCACAACATTCC | GBP5 |
| 125 | | TOP2A |
| 126 | GCATGACCTGGAGCCACGGTCTCTCAGTATCTAAAGTCCTACACAAGGCC | SERP1NB4 |
| 127 | CTACAGGTCCCCTCTGAGCCCTCTCACCTTGTCCTGTGGAAGAAGCACAG | GNLY |
| 128 | GGGGCACAACTCACTACTCTGACAACAACAGCCCTGCGAGCAACATAGTT | GTSF1 |
| 129 | CTGACTGCCCAGGAATCAAGAAGTGCTGTGAAGGCTCTTGCGGGATGGCC | PI3 |
| 130 | AGAGTTCTGACCAGCACCAGATAAGCTTCAGTGCTCTCCTTTCTTTGGCC | S100A7A |
| 131 | CGCCTGTGTGAAAGCTCTGGTCTCCCTGAGGAGCTACCATCTGCAAATCG | IDO1 |
| 132 | GCTGCGTCATAAGGAGACTTCTGTCTTCTCCAGAAGGCAATACCAACCTG | GJB6 |
| 133 | AAAACAGCACTGCCTTCCGCGCTGCCCCAGCTTGCCCCATTCCTTGTCCG | CALML3 |
| 134 | TGACCGGGAGCCGCGGTCTCGTGCTATCTGGAGTCCTACACAAGGCCTTT | SERPINB3 |
| 135 | GTGTGCTGTTGAGGGAGGTATCCTGTTGTTCTTACTCACTCTTCTCATAA | CXCL6 |
| 136 | TGTTCAAGTCCTAGTCTATAGGATTGGCAGTTTAAATGCTTTACTCCCCC | OLFM4 |
| 137 | TGAGTGGAGGCGAACCACTGAGCCAAGGAGCTGGTAGTTACGTTGTCCGC | TCN1 |
| 138 | GAGGGACCCTTGGCTCCTGTGTCTGGTCCACACACCACAGAAGCTTGTAT | VSNL1 |
| 139 | CCTCCTCCAGGTGCGAAGGTCCAGCTCAGTGGCACAAGTGAAAGCAATGA | UBD |
| 140 | GCTGGTGAAACCCCGAAGATCAACACGCTTCAAACTCAGCCCCTTGGAAC | AIM2 |
| 141 | CGAGTACACACTATTCTGACGGCAGACCTGGTTATTGTGATGAAGCGAGG | ABCC9 |
| 142 | CTAGGTTCACCAGTTGAGGGACATTTGGATTGTTCCCACTTCTTGGGCTG | SERPINB13 |
| 143 | CTGATTCCTGCAAGCCAGCAGCCAAAGGAGAATAAGACCTCTGAAGACCC | INDO |
| 144 | ACCACATTCTTTGGTTCCCAGGAGAGCCCCATTCCCAGCCCCTGGTCTCC | KRT5 |
| 145 | TGCTGCTGGAAGCCTCCAAAGTACTTAGTGTCTATTGTTTCCCCTGTGTG | LOC100130897 |
| 146 | GTGGACACAGATCCCACTGGAAGATCCCCTCTCCTGCCCAAGCACTTCAC | KRT14 |
| 147 | CAGCCTGGTCACCTCCTGAGGAATAAATGCTGAACCTCACAAGCCCCATC | FAM83A |
| 148 | GCTGGCTTCTGTAGCCACCTGTCCCTTCTATTTTTCAGCGAAGGTCAGTG | FAM181B |
| 149 | CCTGTGGCAAGCCAGCAAGATGGCCCTGGTGACAGCAAAAGAAACTGCAC | |
| 150 | ACAGGAAGCAAACTAAGCCCCCGCTGTAATGAAACACCTTCTCTGGAGCC | GZMB |
| 151 | CAGAAAGGGTGATCTGTCCCATTTCCAGTGTTCCTGGCAACCTAGCTGGC | DSG3 |
| 152 | AGAAACTCGTGGAGGGGCTGTCCGCTCTGGTGGTGGACGTTAAGTTCGGA | TYMP |
| 153 | GTGTTGTGAACCCCCACCCAGGCAGTATCCATGAAAGCACAAGTGACTAG | KRT6A |
| 154 | CTCTTGCAGTGTCCCTGAATGGCAAGTGATGTACCTTCTGATGCAGTCTG | KRT6B |
| 155 | GGACTTCAGCCAACAGGATTCCTGAGCTGAAATGCAGATGACCACATTCA | HLA-DRB1 |
| 156 | CCACATCGTCTTCCCTGTCCCAATCGACCAGTGTATCGACGGCTGAGTGC | LCN2 |
| 157 | CCAGGATGATCTTCTGTGCTGGGACAGGGACTCTGCCTCTTGGAGTTTGG | KRT4 |
| 158 | TGGAAGATCAGACCCAGCTCCTTACCCTTGTCTGCCAGTTGTACCAGGGC | IFI30 |
| 159 | CTACAGGCGCCTGCTGGAGGGCGAGGAGCATAGGCTGTGTGAAGGTGTTG | LOC100134370 |
| 160 | CCCCGTTTATCCATGTGTCCATTGACGGCCATCTATGTTGCTTCTTCGGC | KIAA1618 |
| 161 | TAACTTCCAGGAGTTCCTCATTCTGGTGATAAAGATGGGCGTGGCAGCCC | S100A8 |
| 162 | GCTCACTTCGATGAGGATGAACGCTGGACGGATGGTAGCAGTGTAGGGAT | MMP7 |
| 163 | GCAACTCATGAACTTGGCCATTCTTTGGGTATGGGACATTCCTCTGATCC | MMP7 |
| 164 | GCTCCACCTTCATCTTCTCATCAAAGCCTACCATGGATACACAGGGAGCT | SPRR2A |
| 165 | GATGAGCTTTGTCTACTTCAAAAGTTTGTTTGCTTACCCCTTCAGCCTCC | GJB2 |

### Cancer Therapeutics

In some embodiments, the cancer cell may be targeted specifically with a therapeutic based upon the differentially expressed gene or gene product. For example, in some embodiments, the differentially expressed gene product is an enzyme, which can convert a anticancer prodrug into its active form. Therefore, in normal cells, where the differentially expressed gene product is not expressed or expressed at significantly lower levels, the prodrug is either not activated or activated in a lesser amount, and is, therefore less toxic to normal cells. Therefore, the cancer prodrug can, in some embodiments, be given in a higher dosage so that the cancer cells can metabolize the prodrug, which will, for example, kill the cancer cell, and the normal cells will not metabolize the prodrug or not as well, and, therefore, be less toxic to the patient. An example of this is where tumor cells overexpress a metalloprotease, which is described in Atkinson et al., British Journal of Pharmacology (2008) 153, 1344-1352. Using proteases to target cancer cells is also described in Carl et al., PNAS, Vol. 77, No. 4, pp. 2224-2228, April 1980. For example, doxorubicin or other type of chemotherapeutic can be linked to a peptide sequence that is specifically cleaved or recognized by the differentially expressed gene product. The doxorubicin or other type of chemotherapeutic is then cleaved from the peptide sequence and is activated such that it can kill or inhibit the growth of the cancer cell whereas in the normal cell the chemotherapeutic is never internalized into the cell or is not metabolized as efficiently, and is, therefore, less toxic.

In some embodiments, a method of treating cancer may comprise gene knockdown of one or more cancer associated sequences described herein. Gene knockdown refers to techniques by which the expression of one or more of an organism's genes is reduced, either through genetic modification (a change in the DNA of one of the organism's chromosomes such as, without limitation, chromosomes encoding cancer associated sequences) or by treatment with a reagent such as a short DNA or RNA oligonucleotide with a sequence complementary to either an mRNA transcript or a gene. In some embodiments, the oligonucleotide used may be selected from RNase-H competent antisense, such as, without limitation, ssDNA oligonucleotides, ssRNA oligonucleotides, phosphorothioate oligonucleotides, or chimeric oligonucleotides; RNase-independent antisense, such as morpholino oligonucleotides, 2′-O-methyl phosphorothioate oligonucleotides, locked nucleic acid oligonucleotides, or peptide nucleic acid oligonucleotides; RNAi oligonucleotides, such as, without limitation, siRNA duplex oligonucleotides, or shRNA oligonucleotides; or any combination thereof. In some embodiments, a plasmid may be introduced into a cell, wherein the plasmid expresses either an antisense RNA transcript or an shRNA transcript. The oligo introduced or transcript expressed may interact with the target mRNA (ex. SEQ ID NOs. 1-55) by complementary base pairing (a sense-antisense interaction).

The specific mechanism of silencing may vary with the oligo chemistry. In some embodiments, the binding of a oligonucleotide described herein to the active gene or its transcripts may cause decreased expression through blocking of transcription, degradation of the mRNA transcript (e.g. by small interfering RNA (siRNA) or RNase-H dependent antisense) or blocking either mRNA translation, pre-mRNA splicing sites or nuclease cleavage sites used for maturation of other functional RNAs such as miRNA (e.g. by Morpholino oligonucleotides or other RNase-H independent antisense). For example, RNase-H competent antisense oligonucleotides (and antisense RNA transcripts) may form duplexes with RNA that are recognized by the enzyme RNase-H, which cleaves the RNA strand. As another example, RNase-independent oligonucleotides may bind to the mRNA and block the translation process application. In some embodiments, the oligonucleotides may bind in the 5′-UTR and halt the initiation complex as it travels from the 5′-cap to the start codon, preventing ribosome assembly. A single strand of RNAi oligonucleotides may be loaded into the RISC complex, which catalytically cleaves complementary sequences and inhibits translation of some mRNAs bearing partially-complementary sequences. The oligonucleotides may be introduced into a cell by any technique including, without limitation, electroporation, microinjection, salt-shock methods such as, for example, CaCl2 shock; transfection of anionic oligo by cationic lipids such as, for example, Lipofectamine; transfection of uncharged oligonucleotides by endosomal release agents such as, for example, Endo-Porter; or any combination thereof. In some embodiments, the oligonucleotides may be delivered from the blood to the cytosol using techniques selected from nanoparticle complexes, virally-mediated transfection, oligonucleotides linked to octaguanidinium dendrimers (Morpholino oligonucleotides), or any combination thereof.

In some embodiments, a method of treating bladder cancer may comprise treating cells to knockdown or inhibit expression of a gene encoding the mRNA disclosed in SEQ ID NOs. 1-55. The method may comprise culturing hES cell-derived clonal embryonic progenitor cell lines CM02 and EN13 (see U.S. Patent Publication 20080070303, entitled “Methods to accelerate the isolation of novel cell strains from pluripotent stem cells and cells obtained thereby”; and U.S. patent application Ser. No. 12/504,630 filed on Jul. 16, 2009 and titled “Methods to Accelerate the Isolation of Novel Cell Strains from Pluripotent Stem Cells and Cells Obtained Thereby”, each of which is incorporated by reference herein in its entirety) with a retrovirus expressing silencing RNA directed to a cancer-associated sequence. In some embodiments, the method may further comprise confirming down-regulation by qPCR. In some embodiments, the method further comprises cryopreserving the cells. In some embodiments, the method further comprises reprogramming the cells. In some embodiments, the method comprises cryopreserving or reprogramming the cells within two days by the exogenous administration of OCT4, MYC, KLF4, and SOX2 (see Takahashi and Yamanaka 2006 Aug. 25; 126(4):663-76; U.S. patent application Ser. No. 12/086,479, published as US2009/0068742 and entitled “Nuclear Reprogramming Factor”) and by the method described in PCT/US06/30632, published as WO/2007/019398 and entitled “Improved Methods of Reprogramming Animal Somatic Cells”. In some embodiments, the method may comprise culturing mammalian differentiated cells under conditions that promote the propagation of ES cells. In some embodiments, any convenient ES cell propagation condition may be used, e.g., on feeders or in feeder free media capable of propagating ES cells. In some embodiments, the method comprises identifying cells from ES colonies in the culture. Cells from the identified ES colony may then be evaluated for ES markers, e.g., Oct4, TRA 1-60, TRA 1-81, SSEA4, etc., and those having ES cell phenotype may be expanded. Control lines that have not been preconditioned by the knockdown may be reprogrammed in parallel to demonstrate the effectiveness of the preconditioning.

In some embodiments, a method for treating bladder cancer comprises administering to a subject in need thereof a therapeutic agent modulating the activity of a cancer associated protein (CAP), wherein said CAP is encoded by a nucleic acid comprising a nucleic acid sequence selected from the group consisting of the human nucleic acid sequences in Table 2 and further wherein the therapeutic agent binds to the cancer associated protein; wherein the cancer associated protein is selected from the group consisting of SEQ ID NOs: 56-110 shown in Table 3.

In some embodiments, a method of treating bladder cancer comprises administering an antibody (e.g. monoclonal antibody, human antibody, humanized antibody, chimeric antibody, and the like) that specifically binds to a cancer associated protein (CAP) that is expressed on a cell surface, wherein the cancer associated protein is selected from the group consisting of SEQ ID NOs: 56-110. In some embodiments, the antibody binds to an extracellular domain of the cancer associated protein. In some embodiments, the antibody binds to a cancer associated protein differentially expressed on a cancer cell surface relative to a normal cell surface, or, in some embodiments, to at least one human cancer cell line. In some embodiments, the antibody is linked to a therapeutic agent. Kits and pharmaceutical compositions for detecting a presence or an absence of cancer cells in a subject, and comprising such antibodies are also provided.

In some embodiments the invention provides a method for inhibiting growth of cancer cells in a subject. In some embodiments, the method comprises administering to the subject an effective amount of a pharmaceutical composition as described herein. In some embodiments the invention provides a method for delivering a therapeutic agent to cancer cells in a subject, the method comprising: administering to the subject an effective amount of a pharmaceutical composition according to according to the invention.

### Methods of Analyzing a Sample

The pattern of gene expression in a particular living cell may be characteristic of its current state. Nearly all differences in the state or type of a cell are reflected in the differences in RNA levels of one or more genes. Comparing expression patterns of uncharacterized genes may provide clues to their function. High throughput analysis of expression of hundreds or thousands of genes can help in (a) identification of complex genetic diseases, (b) analysis of differential gene expression over time, between tissues and disease states, and (c) drug discovery and toxicology studies. Increase or decrease in the levels of expression of certain genes correlate with cancer biology. For example, oncogenes are positive regulators of tumorigenesis, while tumor suppressor genes are negative regulators of tumorigenesis. (Marshall, Cell, 64: 313-326 (1991); Weinberg, Science, 254: 1138-1146 (1991)). Accordingly, some embodiments herein provide for polynucleotide and polypeptide sequences involved in cancer and, in particular, in oncogenesis.

Oncogenes are genes that can cause cancer. Carcinogenesis can occur by a wide variety of mechanisms, including infection of cells by viruses containing oncogenes, activation of protooncogenes in the host genome, and mutations of protooncogenes and tumor suppressor genes. Carcinogenesis is fundamentally driven by somatic cell evolution (i.e. mutation and natural selection of variants with progressive loss of growth control). The genes that serve as targets for these somatic mutations are classified as either protooncogenes or tumor suppressor genes, depending on whether their mutant phenotypes are dominant or recessive, respectively.

The detection of the expression level of the one or more markers disclosed infra may be by any means known in the art. For example where the marker is a protein associated with breast cancer an ELISA may used to detect the expression level of the marker. Other suitable assays for detecting the presence of a protein marker include a radio-immunoassay, a western blot, an immunoprecipitation assay, such as a bead based assay, e.g. a magnetic bead based assay. In some embodiments the marker may be isolated from the sample before detection, but in other embodiments it is not isolated from the sample. In some embodiments the protein marker may be expressed in a cellular context (i.e., on the surface of the cell or within the cell). In these instances immunoecytochemistry may be used to detect the marker. Alternatively, the flow cytometry can used to detect the marker. Where the marker is contained within the cell, the cells may be treated with a detergent to make the marker accessible to a detection reagent. Suitable detection reagents would include any molecule that specifically binds the marker, such as an antibody that specifically binds to an epitope on the marker.

Suitable agents for detecting a protein marker as disclosed infra include any specific binding partner of the breast cancer marker. For example the specific binding partner may be a protein that binds the breast cancer marker, such as an antibody. Other suitable specific binding partners may include a receptor that binds the breast cancer marker or an enzyme that specifically binds the breast cancer marker.

The cancer can also be diagnosed to a specific tissue type as well by visualizing the labeled molecule. The molecule can be visualized or detected using any method, such as but not limited to, MRI, CAT scan, PET scan, and the like. In some embodiments, an antibody can bind to the protein and then be detected. In some embodiments, the level of antibody binding can be quantified to determine whether the protein is overexpressed. Differential expression can also be determined by known methods. Accordingly, embodiments hereof provide a method for imaging structures in tissues and cells of a subject having cancer, is suspected of having cancer, or is undergoing a diagnostic procedure to determine if the person has cancer. If the imaging demonstrates that the cancer associated protein is overexpressed or differentially expressed then the patient is diagnosed as having cancer or suspected of having cancer. Other tests can also be done, such as but not limited to, a biopsy to confirm, or otherwise aid, the diagnosis.

The label molecules can also be labeled by, but not limited to, any radioisotopes that can be imaged with a PET or SPECT camera. For example, radiopharmaceuticals of various embodiments may be radiolabeled with radioisotopes such as, but not limited to, ⁷⁶Br, ¹²³I, ¹²⁵I, ¹³¹I, ^{99m}Tc, ¹¹C, ¹⁸F, or other gamma- or positron-emitting radionuclides. In other embodiments, the label molecules may be radiolabeled with a combination of radioisotopes.

In some embodiments the marker associated with breast cancer may be a nucleic acid, e.g. an mRNA molecule. The nucleic acid may be isolated from the sample. Detection of the nucleic acid may be by any means known in the art. For example the nucleic acid molecule may be detected by Southern blot or northern blot mass spectroscopy, microarray and the like. The nucleic acid may be detected using PCR, for example where the nucleic acid is an RNA molecule, such as an mRNA molecule, rtPCR may be used. The PCR may be quantitative PCR (e.g. qPCT) or real time PCR. The nucleic acid may be detected by in situ hybridization where the sample includes breast cancer cells.

The assays described above may include the use of a probe to detect the nucleic acid marker. Probes are described infra. Briefly, the probe may be a nucleic acid molecule ranging from 5-40, 10-35, 15-30 nucleotides long. The probe may be about 5, about 10, about 20, about 25, about 30, about 35 nucleotides long. The probe may include a portion of a gene encoding the breast cancer marker, or a complement of a gene encoding a breast cancer marker.

It will be appreciated that there are various methods of obtaining expression data and uses of the expression data. For example, the expression data that can be used to detect or diagnose a subject with cancer can be obtained experimentally. In some embodiments, obtaining the expression data comprises obtaining the sample and processing the sample to experimentally determine the expression data. The expression data can comprise expression data for one or more of the cancer associated sequences described herein. The expression data can be experimentally determined by, for example, using a microarray or quantitative amplification method such as, but not limited to, those described herein. In some embodiments, obtaining expression data associated with a sample comprises receiving the expression data from a third party that has processed the sample to experimentally determine the expression data.

The use of microarray analysis of gene expression allows the identification of sequences associated with cancer. These sequences may then be used in a number of different ways, including diagnosis, prognosis, screening for modulators (including both agonists and antagonists), antibody generation (for immunotherapy and imaging), etc. However, as will be appreciated by those skilled in the art, sequences that are identified in one type of cancer may have a strong likelihood of being involved in other types of cancers as well. Thus, while the sequences outlined herein are initially identified as correlated with bladder cancer, they may also be found in other types of cancers as well.

The comparison of gene expression on an mRNA level using Illumina gene expression microarrays hybridized to RNA probe sequences (SEQ ID NOs: 111-165, shown in Table 4) prepared from the diverse categories of cell types: 1) human embryonic stem (“ES”) cells, or gonadal tissues 2) ES, iPS, or EC-derived clonal embryonic progenitor (“EP”) cell lines, 3) nucleated blood cells including but not limited to CD34+ cells and CD133+ cells; 4) Normal mortal somatic adult-derived tissues and cultured cells including: skin fibroblasts, vascular endothelial cells, normal non-lymphoid and non-cancerous tissues, and the like, and 5) malignant cancer cells including cultured cancer cell lines or human tumor tissue and filters was performed to detect genes that are generally expressed (or not expressed) in categories 1, 3, and 5, or categories 1 and 5 but not expressed (or expressed) in categories 2 and 4. Therapies in these cancers based on this observation would be based on reducing the expression of the above referenced transcripts up-regulated in cancer, or otherwise reducing the expression of the gene products.

Gene Expression Assays: Measurement of the gene expression levels may be performed by any known methods in the art, including but not limited to quantitative PCR, or microarray gene expression analysis, bead array gene expression analysis and Northern analysis. The gene expression levels may be represented as relative expression normalized to the ADPRT (Accession number NM_{—}001618.2), GAPD (Accession number NM_{—}002046.2), or other housekeeping genes known in the art. In the case of microarrayed probes of mRNA expression, the gene expression data may also be normalized by a median of medians method. In this method, each array gives a different total intensity. Using the median value is a robust way of comparing cell lines (arrays) in an experiment. As an example, the median was found for each cell line and then the median of those medians became the value for normalization. The signal from the each cell line was made relative to each of the other cell lines.

RNA extraction. Cells from a suitable subject may be incubated with 0.05% trypsin and 0.5 mM EDTA, followed by collecting in DMEM (Gibco, Gaithersburg, Md.) with 0.5% BSA. Total RNA is purified from cells using the RNeasy Mini kit (Qiagen, Hilden, Germany).

Isolation of total and miRNA may be obtained from human embryonic stem cells and differentiated progeny cells. Total RNA or samples enriched for small RNA species were isolated from cell cultures that underwent serum starvation prior to harvesting RNA to approximate cellular growth arrest observed in many mature tissues. Cellular growth arrest was performed by changing to medium containing 0.5% serum for 5 days, with one medium change 2-3 days after the first addition of low serum medium. RNA were harvested according to the vendors instructions for Qiagen RNEasy kits to isolate total RNA or Ambion mirVana kits to isolate RNA enriched for small RNA species. The RNA concentrations were determined by spectrophotometry and RNA quality determined by denaturing agarose gel electrophoresis to visualize 28S and 18S RNA. Samples with clearly visible 28S and 18S bands without signs of degradation and at a ratio of approximately 2:1, 28S:18S, were used for subsequent miRNA analysis.

Assay for miRNA in samples isolated from human embryonic stem cells and differentiated progeny cells. The miRNAs were quantitated using a Human Panel TaqMan MicroRNA Assay from Applied Biosystems, Inc. This is a two-step assay that uses stem-loop primers for reverse transcription (RT) followed by real-time TaqMan®. A total of 330 miRNA assays were performed to quantitate the levels of miRNA in the H9 human embryonic stem cell line, a differentiated fibroblast cell line, and nine cell lines differentiated from human embryonic stem cells. The assay includes two steps, reverse transcription (RT) and quantitative PCR. Real-time PCR was performed on an Applied Biosystems 7500 Real-Time PCR System. The copy number per cell was estimated based on the standard curve of synthetic mir-16 miRNA and assuming a total RNA mass of approximately 15 pg/cell.

The reverse transcription reaction was performed using 1× cDNA archiving buffer, 3.35 units MMLV reverse transcriptase, 5 mM each dNTP, 1.3 units AB RNase inhibitor, 2.5 nM 330-plex reverse primer (RP), 3 ng of cellular RNA in a final volume of 5 μl. The reverse transcription reaction was performed on a BioRad or MJ thermocycler with a cycling profile of 20° C. for 30 sec; 42° C. for 30 see; 50° C. for 1 see, for 60 cycles followed by one cycle of 85° C. for 5 min.

Real-time PCR. Two microlitres of 1:400 diluted Pre-PCR product is used for a 20 ul reaction. All reactions are duplicated. Because the method is very robust, duplicate samples are sufficient and accurate enough to obtain values for miRNA expression levels. TaqMan universal PCR master mix of ABI is used according to manufacturer's suggestion. Briefly, 1× TaqMan Universal Master Mix (ABI), 1 uM Forward Primer, 1 uM Universal Reverse Primer and 0.2 uM TaqMan Probe is used for each real-time PCR. The conditions used are as follows: 95° C. for 10 min, followed by 40 cycles at 95° C. for 15 s, and 60° C. for 1 min. All the reactions are run on ABI Prism 7000 Sequence Detection System.

Microarray hybridization and data processing. cDNA samples and cellular total RNA (5 μg in each of eight individual tubes) are subjected to the One-Cycle Target Labeling procedure for biotin labeling by in vitro transcription (IVT) (Affymetrix, Santa Clara, Calif.) or using the Illumina Total Prep RNA Labelling kit. For analysis on Affymetix gene chips, the cRNA is subsequently fragmented and hybridized to the Human Genome U133 Plus 2.0 Array (Affymetrix) according to the manufacturer's instructions. The microarray image data are processed with the GeneChip Scanner 3000 (Affymetrix) to generate CEL data. The CEL data are then subjected to analysis with dChip software, which has the advantage of normalizing and processing multiple datasets simultaneously. Data obtained from the eight nonamplified controls from cells, from the eight independently amplified samples from the diluted cellular RNA, and from the amplified cDNA samples from 20 single cells are normalized separately within the respective groups, according to the program's default setting. The model based expression indices (MBEI) are calculated using the PM/MM difference mode with log-2 transformation of signal intensity and truncation of low values to zero. The absolute calls (Present, Marginal and Absent) are calculated by the Affymetrix Microarray Software 5.0 (MAS 5.0) algorithm using the dChip default setting. The expression levels of only the Present probes are considered for all quantitative analyses described below. The GEO accession number for the microarray data is GSE4309. For analysis on Illumina Human HT-12 v4 Expression Bead Chips, labeled cRNA are hybridized according to the manufacturer's instructions.

Calculation of coverage and accuracy. A true positive is defined as probes called Present in at least six of the eight nonamplified controls, and the true expression levels are defined as the log-averaged expression levels of the Present probes. The definition of coverage is (the number of truly positive probes detected in amplified samples)/(the number of truly positive probes). The definition of accuracy is (the number of truly positive probes detected in amplified samples)/(the number of probes detected in amplified samples). The expression levels of the amplified and nonamplified samples are divided by the class interval of 20.5 (20, 20.5, 21, 21.5 . . . ), where accuracy and coverage are calculated. These expression level bins are also used to analyze the frequency distribution of the detected probes.

Analysis of gene expression profiles of cells. The unsupervised clustering and class neighbor analyses of the microarray data from cells are performed using GenePattern software available online from MIT, which performs the signal-to-noise ratio analysis/T-test in conjunction with the permutation test to preclude the contribution of any sample variability, including those from methodology and/or biopsy, at high confidence. The analyses are conducted on the 14,128 probes for which at least 6 out of 20 single cells provided Present calls and at least 1 out of 20 samples provided expression levels >20 copies per cell. The expression levels calculated for probes with Absent/Marginal calls were truncated to zero. To calculate relative gene expression levels, the Ct values obtained with Q-PCR analyses are corrected using the efficiencies of the individual primer pairs quantified either with whole human genome (BD Biosciences) or plasmids that contain gene fragments. The relative expression levels are further transformed into copy numbers with a calibration line calculated using the spike RNAs included in the reaction mixture (log₁₀ [expression level]=1.05×log₁₀ [copy number]+4.65). The Chi-square test for independence is performed to evaluate the association of gene expressions with Gata4, which represents the difference between cluster 1 and cluster 2 determined by the unsupervised clustering and which is restricted to PE at later stages. The expression levels of individual genes measured with Q-PCR are classified into three categories: high (>100 copies per cell), middle (10-100 copies per cell), and low (<10 copies per cell). The Chi-square and P-values for independence from Gata4 expression are calculated based on this classification. Chi squared is defined as follows: χ2=ΣΣ(n fij−fi fj)²/n fi fj, where i and j represent expression level categories (high, middle or low) of the reference (Gata4) and the target gene, respectively; fi, fj, and fij represent the observed frequency of categories i, j and ij, respectively; and n represents the sample number (n=24). The degrees of freedom are defined as (r−1)×(c−1), where r and c represent available numbers of expression level categories of Gata4 and of the target gene, respectively.

### Expression of Cancer Associated Sequences in Cells

Electroporation may be used to introduce the cancer associated nucleic acids described herein into mammalian cells (Neumann, E. et al. (1982) EMBO J. 1, 841-845), plant and bacterial cells, and may also be used to introduce proteins (Marrero, M. B. et al. (1995) J. Biol. Chem. 270, 15734-15738; Nolkrantz, K. et al. (2002) Anal. Chem. 74, 4300-4305; Rui, M. et al. (2002) Life Sci. 71, 1771-1778). Cells (such as the cells of this invention) suspended in a buffered solution of the purified protein of interest are placed in a pulsed electrical field. Briefly, high-voltage electric pulses result in the formation of small (nanometer-sized) pores in the cell membrane. Proteins enter the cell via these small pores or during the process of membrane reorganization as the pores close and the cell returns to its normal state. The efficiency of delivery may be dependent upon the strength of the applied electrical field, the length of the pulses, temperature and the composition of the buffered medium. Electroporation is successful with a variety of cell types, even some cell lines that are resistant to other delivery methods, although the overall efficiency is often quite low. Some cell lines may remain refractory even to electroporation unless partially activated.

Microinjection may be used to introduce femtoliter volumes of DNA directly into the nucleus of a cell (Capecchi, M. R. (1980) Cell 22, 470-488) where it can be integrated directly into the host cell genome, thus creating an established cell line bearing the sequence of interest. Proteins such as antibodies (Abarzua, P. et al. (1995) Cancer Res. 55, 3490-3494; Theiss, C. and Meller, K. (2002) Exp. Cell Res. 281, 197-204) and mutant proteins (Naryanan, A. et al. (2003) J. Cell Sci. 116, 177-186) can also be directly delivered into cells via microinjection to determine their effects on cellular processes firsthand. Microinjection has the advantage of introducing macromolecules directly into the cell, thereby bypassing exposure to potentially undesirable cellular compartments such as low-pH endosomes.

Several proteins and small peptides have the ability to transduce or travel through biological membranes independent of classical receptor-mediated or endocytosis-mediated pathways. Examples of these proteins include the HIV-1 TAT protein, the herpes simplex virus 1 (HSV-1) DNA-binding protein VP22, and the Drosophila Antennapedia (Antp) homeotic transcription factor. In some embodiments, protein transduction domains (PTDs) from these proteins may be fused to other macromolecules, peptides or proteins such as, without limitation, a cancer associated polypepdtide to successfully transport the polypeptide into a cell (Schwarze, S. R. et al (2000) Trends Cell Biol. 10, 290-295). Exemplary advantages of using fusions of these transduction domains is that protein entry is rapid, concentration-dependent and appears to work with difficult cell types (Fenton, M. et al. (1998) J. Immunol. Methods 212, 41-48.).

In some embodiments, liposomes may be used as vehicles to deliver oligonucleotides, DNA (gene) constructs and small drug molecules into cells (Zabner, J. et al. (1995) J. Biol. Chem. 270, 18997-19007; Felgner, P. L. et al. (1987) Proc. Natl. Acad. Sci. USA 84, 7413-7417). Certain lipids, when placed in an aqueous solution and sonicated, form closed vesicles consisting of a circularized lipid bilayer surrounding an aqueous compartment. The vesicles or liposomes of embodiments herein may be formed in a solution containing the molecule to be delivered. In addition to encapsulating DNA in an aqueous solution, cationic liposomes may spontaneously and efficiently form complexes with DNA, with the positively charged head groups on the lipids interacting with the negatively charged backbone of the DNA. The exact composition and/or mixture of cationic lipids used can be altered, depending upon the macromolecule of interest and the cell type used (Felgner, J. H. et al. (1994) J. Biol. Chem. 269, 2550-2561). The cationic liposome strategy has also been applied successfully to protein delivery (Zelphati, O, et al. (2001) J. Biol. Chem. 276, 35103-35110). Because proteins are more heterogeneous than DNA, the physical characteristics of the protein, such as its charge and hydrophobicity, may influence the extent of its interaction with the cationic lipids.

### Screening Assays for Cancer Drugs

In some embodiments, a method of screening drug candidates includes comparing the level of expression of the cancer-associated sequence in the absence of the drug candidate to the level of expression in the presence of the drug candidate.

Some embodiments are directed to a method of screening for a therapeutic agent capable of binding to a cancer-associated sequence (nucleic acid or protein), the method comprising combining the cancer-associated sequence and a candidate therapeutic agent, and determining the binding of the candidate agent to the cancer-associated sequence.

Further provided herein is a method for screening for a therapeutic agent capable of modulating the activity of a cancer-associated sequence. In some embodiments, the method comprises combining the cancer-associated sequence and a candidate therapeutic agent, and determining the effect of the candidate agent on the bioactivity of the cancer-associated sequence. An agent that modulates the bioactivity of the cancer associate sequence is said to be a therapeutic agent capable of modulating the activity of the cancer-associated sequence

A method of screening for anticancer activity, the method comprising: (a) contacting a cell that expresses a cancer associated gene which transcribes a cancer associated sequence selected from SEQ ID NOs: 1-55, homologs thereof, combinations thereof, or fragments thereof with an anticancer drug candidate; (b) detecting an effect of the anticancer drug candidate on an expression of the cancer associated polynucleotide in the cell; and (c) comparing the level of expression in the absence of said drug candidate to the level of expression in the presence of the drug candidate; wherein an effect on the expression of the cancer associate polynucleotide indicates that the candidate has anticancer activity.

In some embodiments, a method of evaluating the effect of a candidate cancer drug may comprise administering the drug to a patient and removing a cell sample from the patient. The expression profile of the cell is then determined. In some embodiments, the method may further comprise comparing the expression profile of the patient to an expression profile of a healthy individual. In some embodiments, the expression profile comprises measuring the expression of one or more or any combination thereof of the sequences disclosed herein. In some embodiments, where the expression profile of one or more or any combination thereof of the sequences disclosed herein is modified (increased or decreased) the candidate cancer drug is said to be effective.

In some embodiments, the invention provides a method of screening for anticancer activity comprising: (a) providing a cell that expresses a cancer associated gene encoded by a nucleic acid sequence selected from the group consisting of the cancer associated sequences shown in Table 2 (SEQ ID NOs: 1-55), or fragment thereof, (b) contacting the cell, which can be derived from a cancer cell with an anticancer drug candidate; (c) monitoring an effect of the anticancer drug candidate on an expression of the cancer associated sequence in the cell sample, and optionally (d) comparing the level of expression in the absence of said drug candidate to the level of expression in the presence of the drug candidate. The drug candidate may be an inhibitor of transcription, a G-protein coupled receptor antagonist, a growth factor antagonist, a serine-threonine kinase antagonist, a tyrosine kinase antagonist. In some embodiments, where the candidate modulates the expression of the cancer associated sequence the candidate is said to have anticancer activity. In some embodiments, the anticancer activity is determined by measuring cell growth. In some embodiments, the candidate inhibits or retards cell growth and is said to have anticancer activity. In some embodiments, the candidate causes the cell to die, and thus, the candidate is said to have anticancer activity.

In some embodiments, the invention provides a method for screening for a therapeutic agent capable of modulating the activity of a cancer associated sequence, wherein said sequence can be encoded by a nucleic acid comprising a nucleic acid sequence selected from the group consisting of the polynucleotide sequences SEQ ID NOs: 1-55 shown in Table 2, said method comprising: a) combining said cancer associated sequence and a candidate therapeutic agent; and b) determining the effect of the candidate agent on the bioactivity of said cancer associated sequence. According to the method the therapeutic agent: affects the expression of the cancer associated sequence; affects the activity of the cancer associated sequence, wherein such activity is selected from the activities listed in Table 21. In some embodiments, the cancer associated sequence is a cancer associate protein (CAP). In some embodiments, the cancer associated sequence is a cancer associate nucleic acid molecule.

### Pharmaceutical Formulations and Administration

Modes of administration for a therapeutic (either alone or in combination with other pharmaceuticals) can be, but are not limited to, sublingual, injectable (including short-acting, depot, implant and pellet forms injected subcutaneously or intramuscularly), or by use of vaginal creams, suppositories, pessaries, vaginal rings, rectal suppositories, intrauterine devices, and transdermal forms such as patches and creams.

Specific modes of administration will depend on the indication. The selection of the specific route of administration and the dose regimen is to be adjusted or titrated by the clinician according to methods known to the clinician in order to obtain the optimal clinical response. The amount of therapeutic to be administered is that amount which is therapeutically effective. The dosage to be administered will depend on the characteristics of the subject being treated, e.g., the particular animal treated, age, weight, health, types of concurrent treatment, if any, and frequency of treatments, and can be easily determined by one of skill in the art (e.g., by the clinician).

Pharmaceutical formulations containing the therapeutic of the present invention and a suitable carrier can be solid dosage forms which include, but are not limited to, tablets, capsules, cachets, pellets, pills, powders and granules; topical dosage forms which include, but are not limited to, solutions, powders, fluid emulsions, fluid suspensions, semi-solids, ointments, pastes, creams, gels and jellies, and foams; and parenteral dosage forms which include, but are not limited to, solutions, suspensions, emulsions, and dry powder; comprising an effective amount of a polymer or copolymer of the present invention. It is also known in the art that the active ingredients can be contained in such formulations with pharmaceutically acceptable diluents, fillers, disintegrants, binders, lubricants, surfactants, hydrophobic vehicles, water soluble vehicles, emulsifiers, buffers, humectants, moisturizers, solubilizers, preservatives and the like. The means and methods for administration are known in the art and an artisan can refer to various pharmacologic references for guidance. For example, Modern Pharmaceutics, Banker & Rhodes, Marcel Dekker, Inc. (1979); and Goodman & Gilman's The Pharmaceutical Basis of Therapeutics, 6th Edition, MacMillan Publishing Co., New York (1980) can be consulted.

The compositions of the present invention can be formulated for parenteral administration by injection, e.g., by bolus injection or continuous infusion. The compositions can be administered by continuous infusion subcutaneously over a period of about 15 minutes to about 24 hours. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents.

For oral administration, the compositions can be formulated readily by combining the therapeutic with pharmaceutically acceptable carriers well known in the art. Such carriers enable the therapeutic of the invention to be formulated as tablets, pills, dragees, capsules, liquids, gels, syrups, slurries, suspensions and the like, for oral ingestion by a patient to be treated. Pharmaceutical preparations for oral use can be obtained by adding a solid excipient, optionally grinding the resulting mixture, and processing the mixture of granules, after adding suitable auxiliaries, if desired, to obtain tablets or dragee cores. Suitable excipients include, but are not limited to, fillers such as sugars, including, but not limited to, lactose, sucrose, mannitol, and sorbitol; cellulose preparations such as, but not limited to, maize starch, wheat starch, rice starch, potato starch, gelatin, gum tragacanth, methyl cellulose, hydroxypropylmethyl-cellulose, sodium carboxymethylcellulose, and polyvinylpyrrolidone (PVP). If desired, disintegrating agents can be added, such as, but not limited to, the cross-linked polyvinyl pyrrolidone, agar, or alginic acid or a salt thereof such as sodium alginate.

Dragee cores can be provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active therapeutic doses.

Pharmaceutical preparations which can be used orally include, but are not limited to, push-fit capsules made of gelatin, as well as soft, sealed capsules made of gelatin and a plasticizer, such as glycerol or sorbitol. The push-fit capsules can contain the active ingredients in admixture with filler such as, e.g., lactose, binders such as, e.g., starches, and/or lubricants such as, e.g., talc or magnesium stearate and, optionally, stabilizers. In soft capsules, the active therapeutic can be dissolved or suspended in suitable liquids, such as fatty oils, liquid paraffin, or liquid polyethylene glycols. In addition, stabilizers can be added. All formulations for oral administration should be in dosages suitable for such administration.

For buccal administration, the pharmaceutical compositions can take the form of, e.g., tablets or lozenges formulated in a conventional manner.

For administration by inhalation, the therapeutic for use according to the present invention is conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of, e.g., gelatin for use in an inhaler or insufflator can be formulated containing a powder mix of the therapeutic and a suitable powder base such as lactose or starch.

The compositions can also be formulated in rectal compositions such as suppositories or retention enemas, e.g., containing conventional suppository bases such as cocoa butter or other glycerides.

In addition to the formulations described previously, the therapeutic of the present invention can also be formulated as a depot preparation. Such long acting formulations can be administered by implantation (for example subcutaneously or intramuscularly) or by intramuscular injection.

Depot injections can be administered at about 1 to about 6 months or longer intervals. Thus, for example, the compositions can be formulated with suitable polymeric or hydrophobic materials (for example as an emulsion in an acceptable oil) or ion exchange resins, or as sparingly soluble derivatives, for example, as a sparingly soluble salt.

In transdermal administration, the compositions of the present invention, for example, can be applied to a plaster, or can be applied by transdermal, therapeutic systems that are consequently supplied to the organism.

Pharmaceutical compositions can include suitable solid or gel phase carriers or excipients. Examples of such carriers or excipients include but are not limited to calcium carbonate, calcium phosphate, various sugars, starches, cellulose derivatives, gelatin, and polymers such as, e.g., polyethylene glycols.

The compositions can also be administered in combination with other active ingredients, such as, for example, adjuvants, protease inhibitors, or other compatible drugs or compounds where such combination is seen to be desirable or advantageous in achieving the desired effects of the methods described herein.

In some embodiments, the disintegrant component comprises one or more of croscarmellose sodium, carmellose calcium, crospovidone, alginic acid, sodium alginate, potassium alginate, calcium alginate, an ion exchange resin, an effervescent system based on food acids and an alkaline carbonate component, clay, talc, starch, pregelatinized starch, sodium starch glycolate, cellulose floe, carboxymethylcellulose, hydroxypropylcellulose, calcium silicate, a metal carbonate, sodium bicarbonate, calcium citrate, or calcium phosphate.

In some embodiments, the diluent component may include one or more of mannitol, lactose, sucrose, maltodextrin, sorbitol, xylitol, powdered cellulose, microcrystalline cellulose, carboxymethylcellulose, carboxyethylcellulose, methylcellulose, ethylcellulose, hydroxyethylcellulose, methylhydroxyethylcellulose, starch, sodium starch glycolate, pregelatinized starch, a calcium phosphate, a metal carbonate, a metal oxide, or a metal aluminosilicate.

In some embodiments, the optional lubricant component, when present, comprises one or more of stearic acid, metallic stearate, sodium stearylfumarate, fatty acid, fatty alcohol, fatty acid ester, glycerylbehenate, mineral oil, vegetable oil, paraffin, leucine, silica, silicic acid, talc, propylene glycol fatty acid ester, polyethoxylated castor oil, polyethylene glycol, polypropylene glycol, polyalkylene glycol, polyoxyethylene-glycerol fatty ester, polyoxyethylene fatty alcohol ether, polyethoxylated sterol, polyethoxylated castor oil, polyethoxylated vegetable oil, or sodium chloride.

### Kits

In some embodiments, the invention provides a kit for diagnosing the presence of cancer in a test sample, said kit comprising at least one polynucleotide that selectively hybridizes to a cancer associated polynucleotide sequence shown in Table 2, or its complement. In another embodiment the invention provides an electronic library comprising a cancer associated polynucleotide, a cancer associated polypeptide, or fragment thereof, shown in Table 2. In other embodiments the invention provides a kit for diagnosing the presence of cancer in a test sample, said kit comprising at least one polypeptide or protein shown in Table 3. In further embodiments the invention provides at least one polynucleotide that selectively hybridizes to a cancer associated polynucleotide chosen from MMP11, MMP12, COL10A1, FCRLB, SFN, KRT6A, S100A2, S100A7. In still other embodiments the invention provides a plurality of polynucleotide that selectively hybridizes to a cancer associated polynucleotide chosen from MMP11, MMP12, COL10A1, FCRLB, SFN, KRT6A, S100A2, S100A7

The kits and systems for practicing the subject methods, as described above, may be configured to diagnose cancer in a subject, treat cancer in a subject, or perform basic research experiments on cancer cells (e.g., either derived directly from a subject, grown in vitro or ex vivo, or from an animal model of cancer. The various components of the kits may be present in separate containers or certain compatible components may be precombined into a single container, as desired.

The subject systems and kits may also include one or more other reagents for performing any of the subject methods. The reagents may include one or more matrices, solvents, sample preparation reagents, buffers, desalting reagents, enzymatic reagents, denaturing reagents, probes, polynucleotides, vectors (e.g., plasmid or viral vectors), etc., where calibration standards such as positive and negative controls may be provided as well. As such, the kits may include one or more containers such as vials or bottles, with each container containing a separate component for carrying out a sample processing or preparing step and/or for carrying out one or more steps for producing a normalized sample according to the present invention.

In addition to above-mentioned components, the subject kits typically further include instructions for using the components of the kit to practice the subject methods. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or sub-packaging) etc. In other embodiments, the instructions are present as an electronic storage data file present on a suitable computer readable storage medium, e.g. CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

In addition to the subject database, programming and instructions, the kits may also include one or more control samples and reagents, e.g., two or more control samples for use in testing the kit.

### Example 1

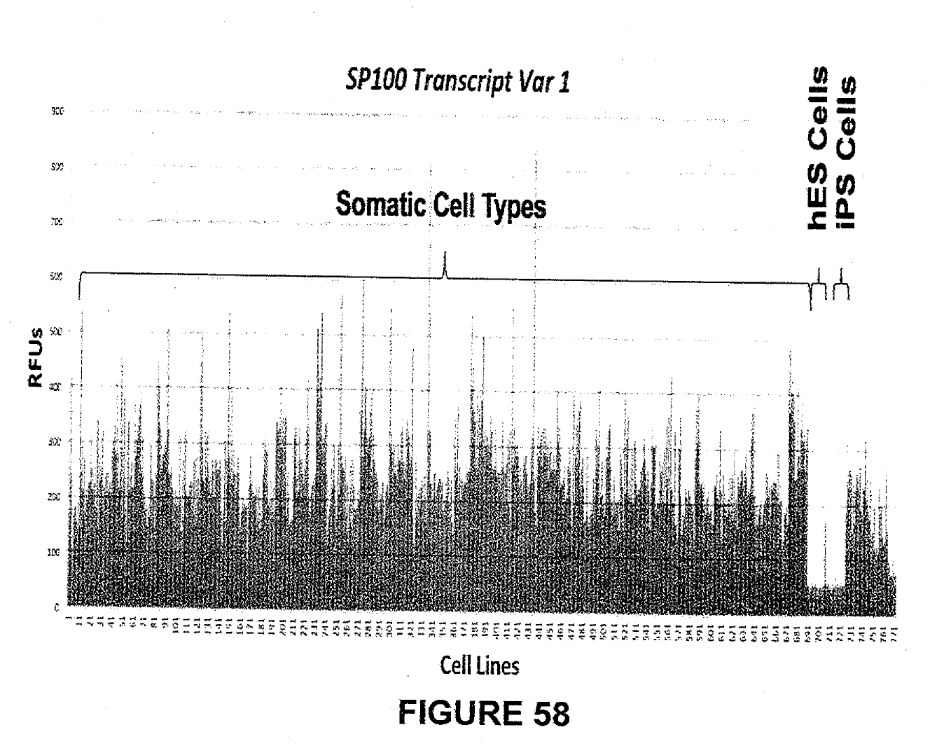

The Differential Expression of SP100—We utilized the screen of the present invention with a large gene expression microarray dataset performed on Illumina microarrays including >400 samples comprised of normal human cell lines including such cell types derived from all three embryonic germ layers as normal human astrocytes, normal human articular chondrocytes, normal bronchial epithelial cells, adult-derived stein cells such as mesenchymal, adipocyte, and dental pulp stem cells, hES-derived clonal embryonic progenitor lines, pluripotent stem (hESCs), hESCs, iPS lines and an EC line. As shown in FIG. 58, SP100 is expressed in essentially all somatic cell types but is not expressed at all in hES cell lines or established iPS cell lines.

### Example 2

Knockdown/inhibition of SP100 expression followed by transcriptional reprogramming accelerates reprogramming while reducing the accumulation of mutations. The hES cell-derived clonal embryonic progenitor cell lines CM02 and EN13 (see U.S. Patent Publication 20080070303, entitled “Methods to accelerate the isolation of novel cell strains from pluripotent stem cells and cells obtained thereby”; and U.S. patent application Ser. No. 12/504,630 filed on Jul. 16, 2009 and titled “Methods to Accelerate the Isolation of Novel Cell Strains from Pluripotent Stem Cells and Cells Obtained Thereby”, each of which is incorporated by reference herein in its entirety) are first cultured with retrovirus expressing silencing RNA directed to SP100 and the down-regulation is confirmed by qPCR. The cells are then cryopreserved or reprogrammed within two days by the exogenous administration of OCT4, MYC, KLF4, and SOX2 (see Takahashi and Yamanaka 2006 Aug. 25; 126(4):663-76; U.S. patent application Ser. No. 12/086,479, published as US2009/0068742 and entitled “Nuclear Reprogramming Factor”, each of which is incorporated herein by reference) and by the method described in PCT/US06/30632, published as WO/2007/019398 and entitled “Improved Methods of Reprogramming Animal Somatic Cells”, incorporated by reference herein in its entirety. The cells may also be conditioned to knockdown/inhibit the expression of the LMNA gene. Control lines that have not been preconditioned by the knockdown of SP100 or LMNA or both SP100 and LMNA are reprogrammed in parallel to demonstrate the shorted time to reprogramming to pluripotency and are sequenced to compare the accumulated mutations in the cells and the lower rate of mutations in the cells preconditioned to lower SP100, LMNA, or both gene products. LMNA expression has been shown previously to be low/absent from ES cells but present in many somatic cells.

### Example 3

Knockdown/inhibition of SP100 expression followed by culturing under conditions for propagating ES cells.—Differentiated mammalian cells (e.g., human cells) are treated to knockdown or inhibit SP100 gene expression (e.g., as described above). The cells may also be treated to knockdown/inhibit the expression of LMNA gene. The cells are cultured under conditions that promote the propagation of ES cells. Any convenient ES cell propagation condition can be used, e.g., on feeders or in feeder free media capable of propagating ES cells. ES colonies are identified in the culture. Cells from the identified ES colony are then evaluated for ES markers, e.g., Oct4, TRA 1-60, TRA 1-81, SSEA4, etc., and those having ES cell phenotype are expanded. In certain embodiments, LMNA-negative cells are used in the above protocol, such as peripheral mononuclear cells (e.g., CD34+ or CD133+ cells). Control lines that have not been preconditioned by the knockdown of SP100 or LMNA or both SP00 and LMNA can be reprogrammed in parallel to demonstrate the effectiveness of the preconditioning.

### Example 4

Additional genes differentially expressed in normal versus diverse cancer types. RNA was obtained from cultured diverse cultured human cell types, normal human tissues, and malignant human tumors and analyzed on Illumina gene expression microarrays. As shown in Table VI, genes are easily identified that provide novel diagnostics for cancer and targets for cancer therapy.

### Example 5

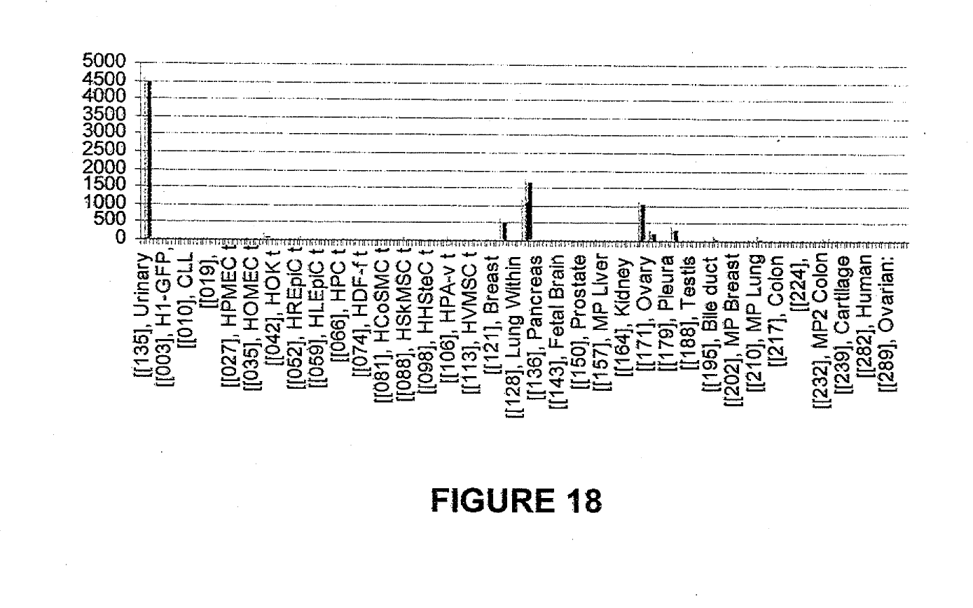

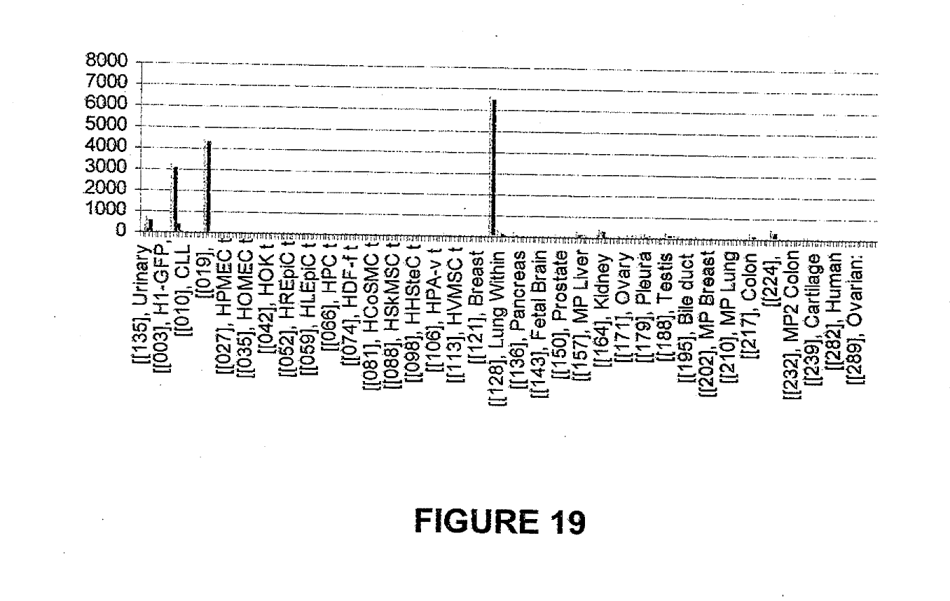

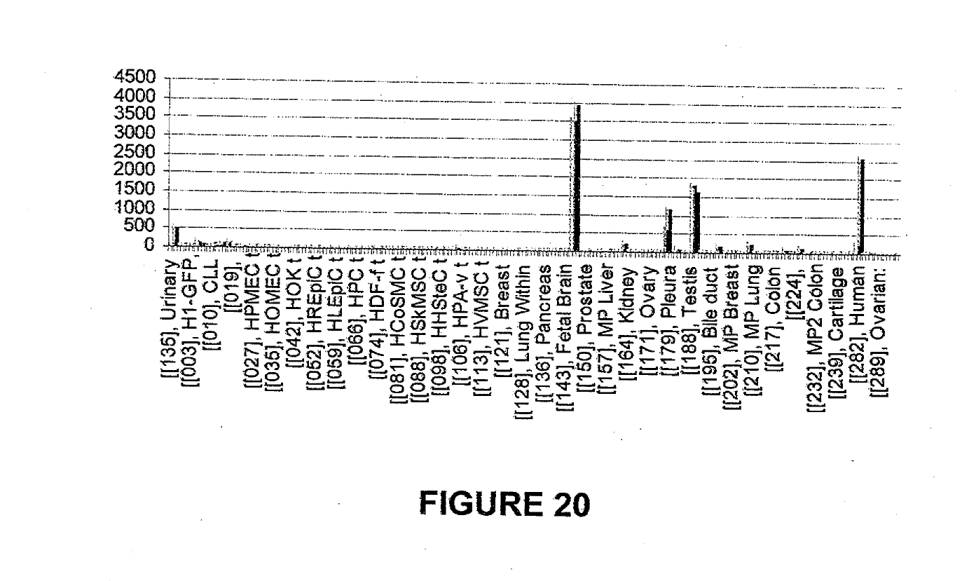

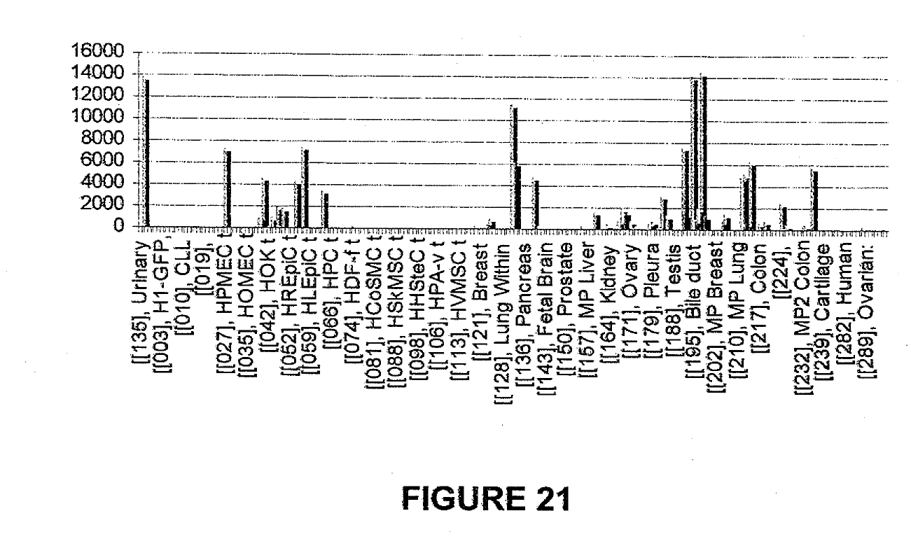

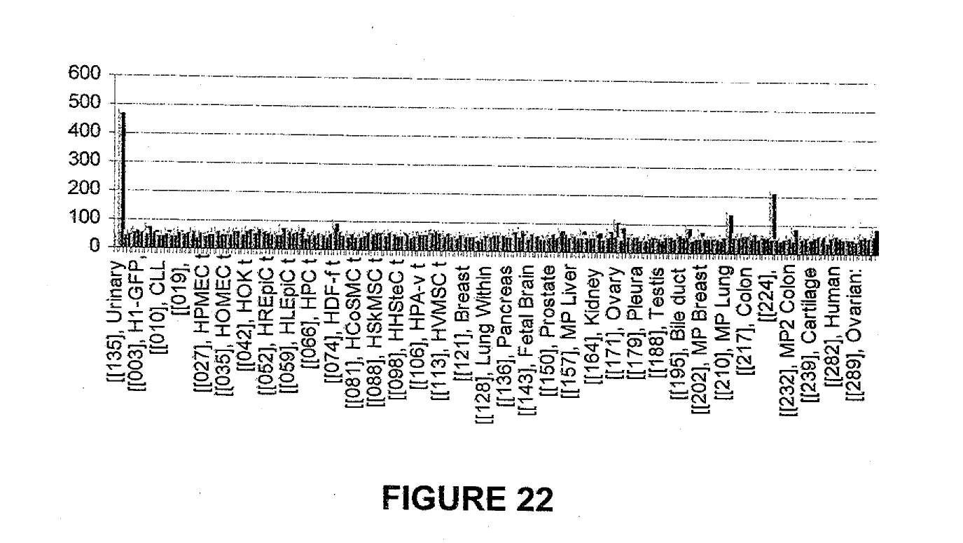

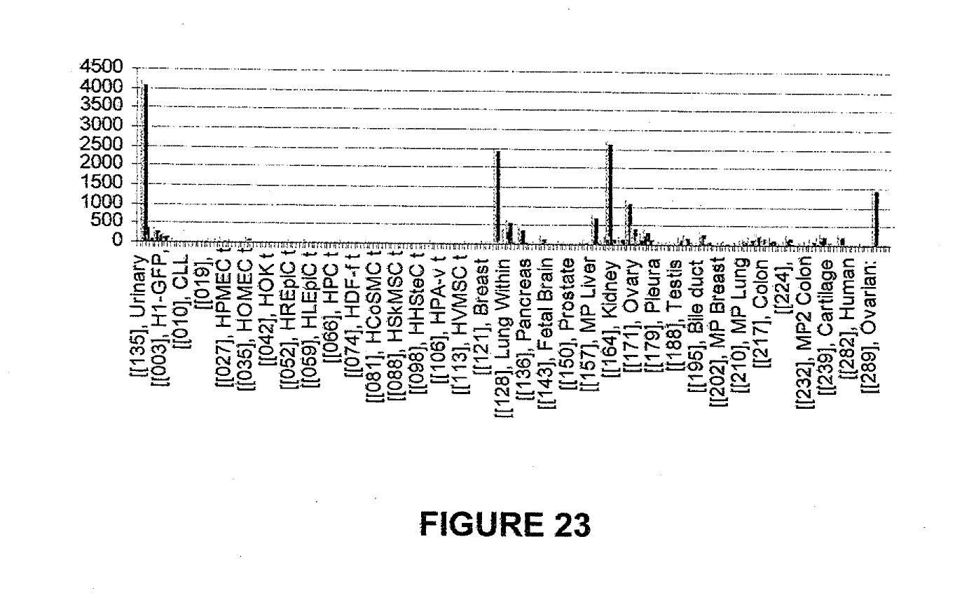

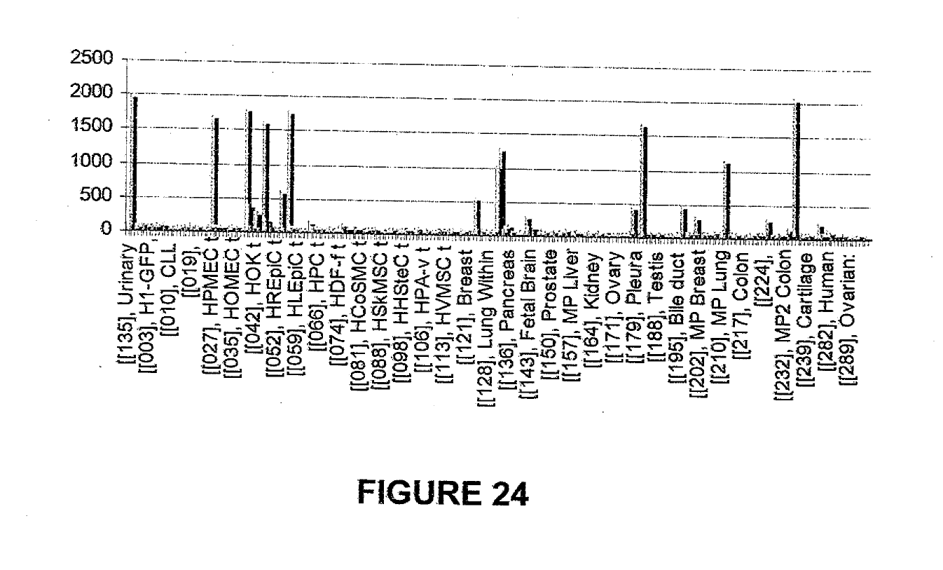

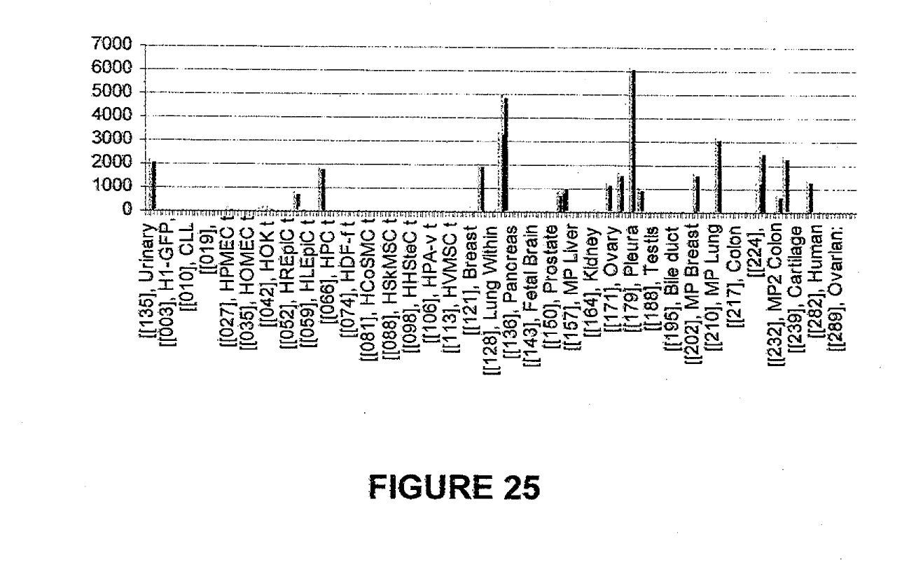

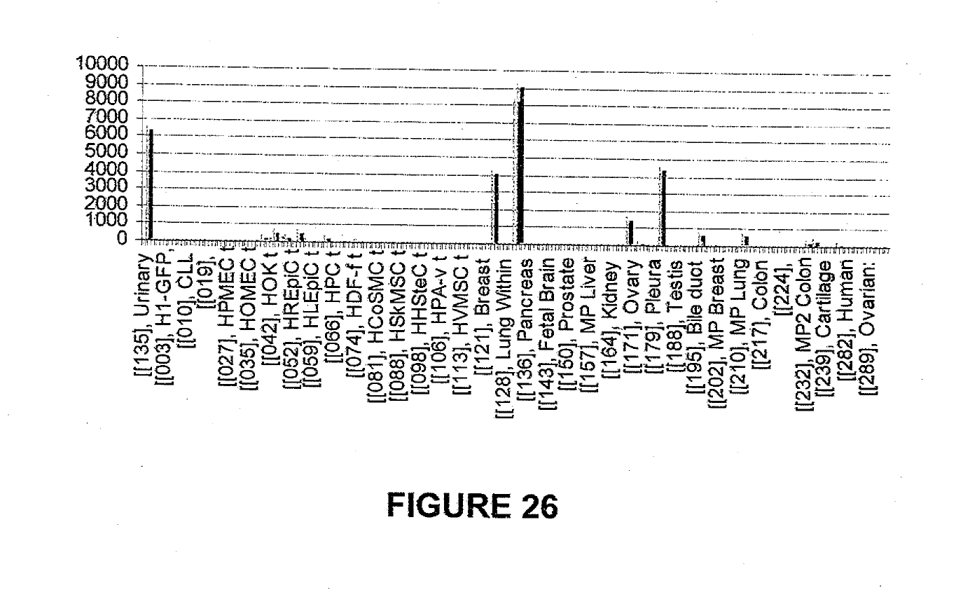

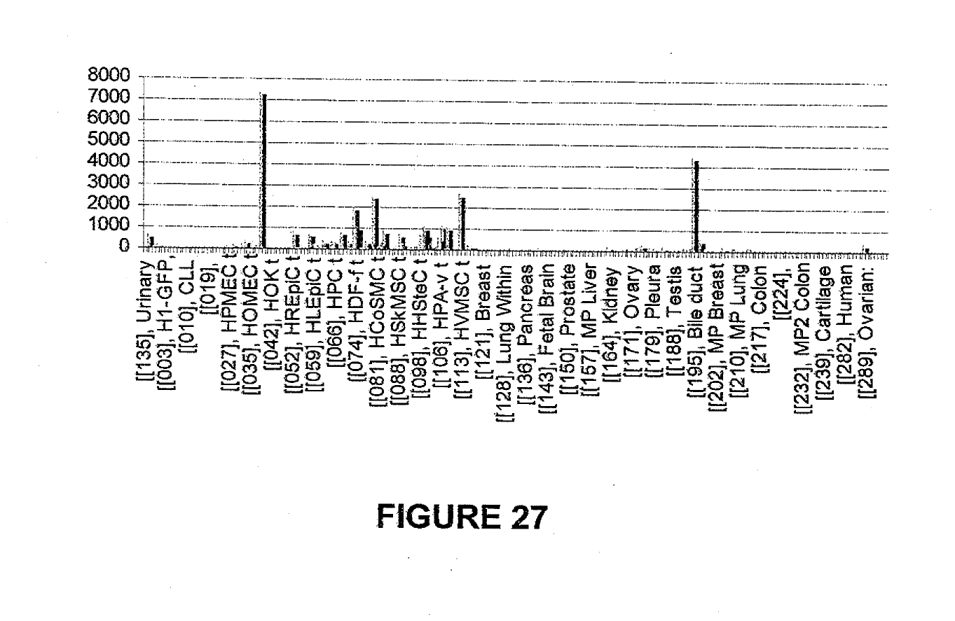

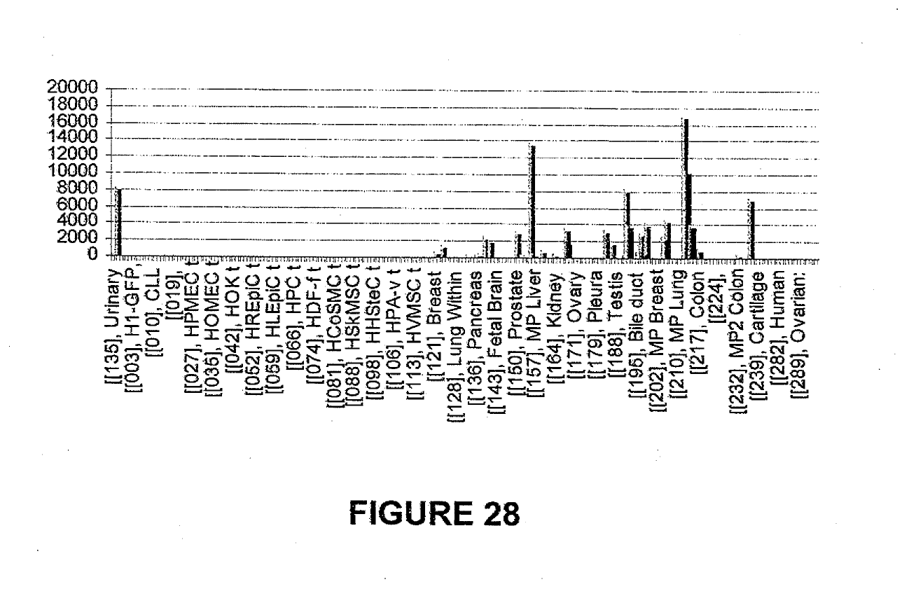

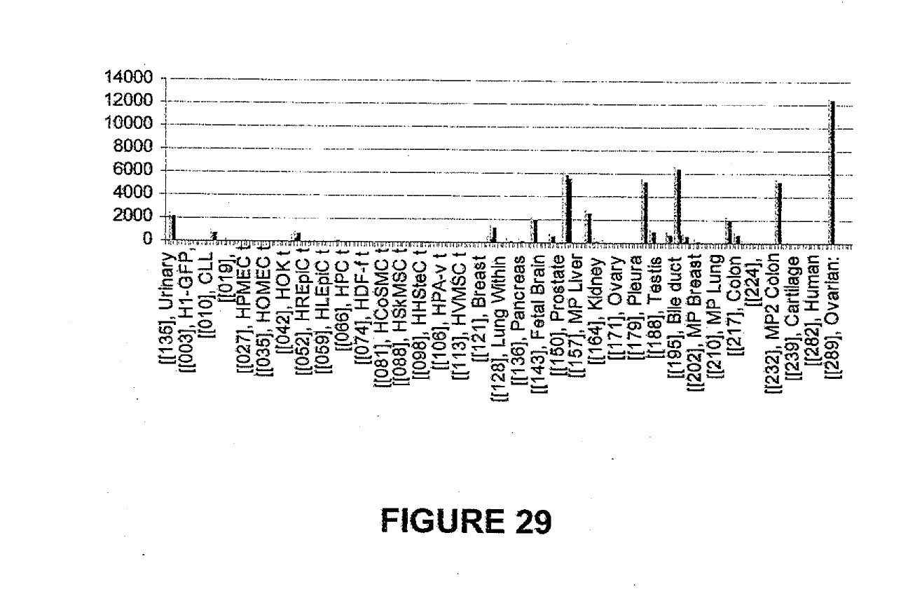

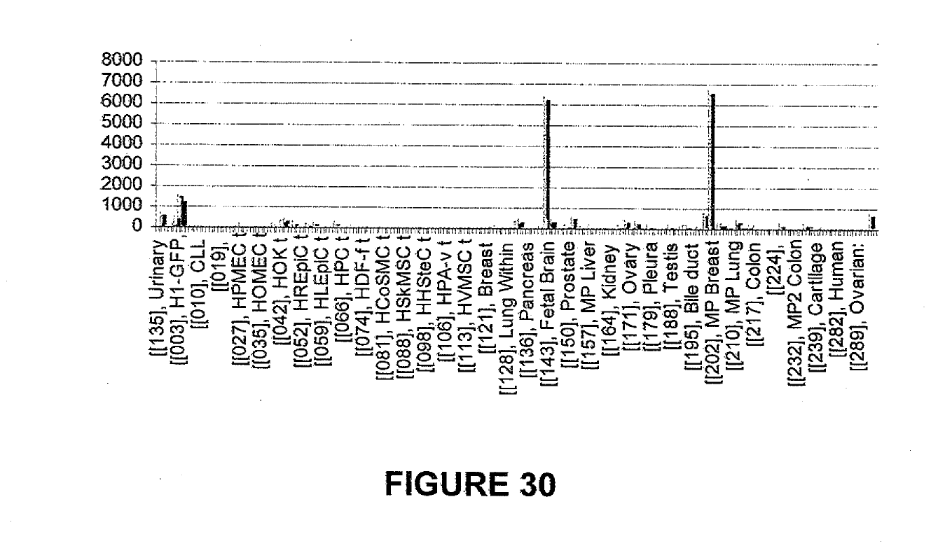

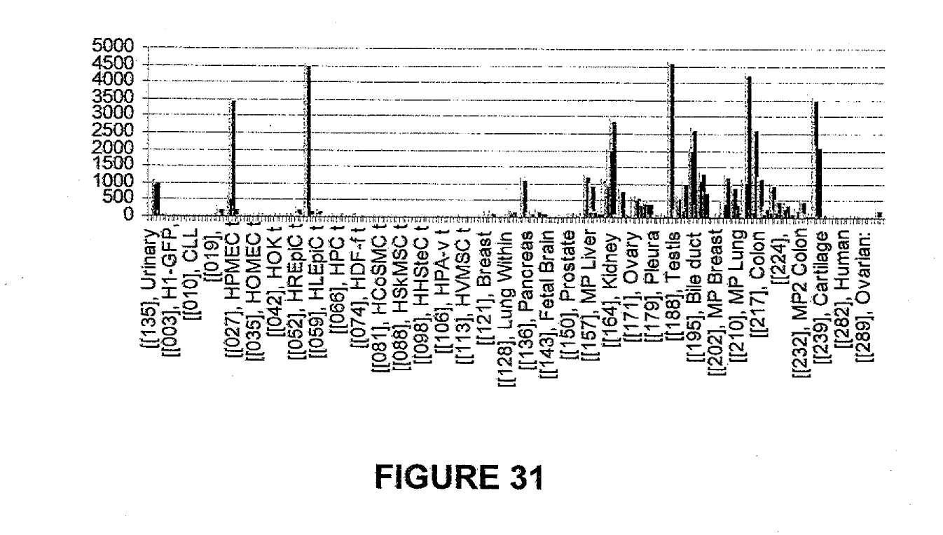

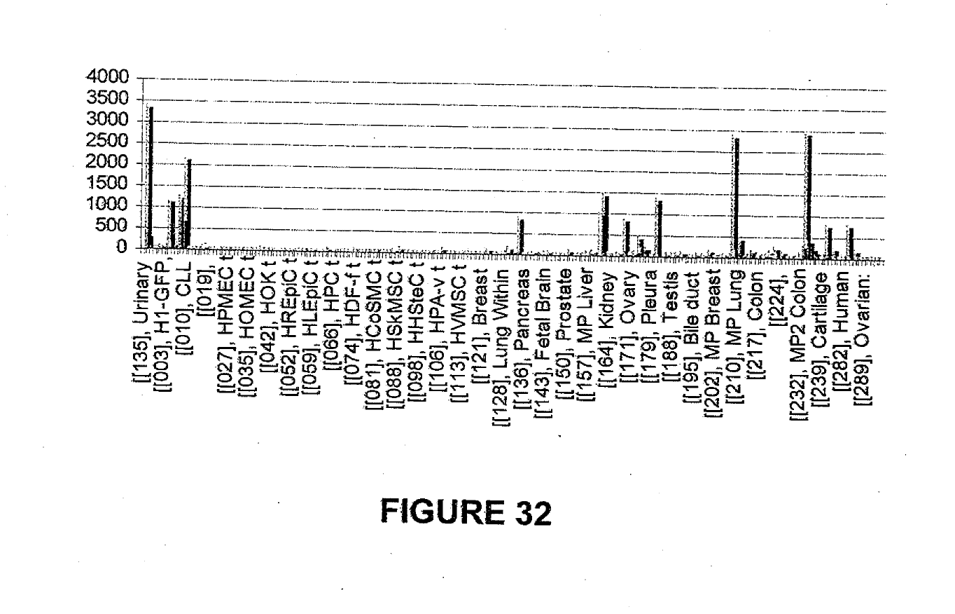

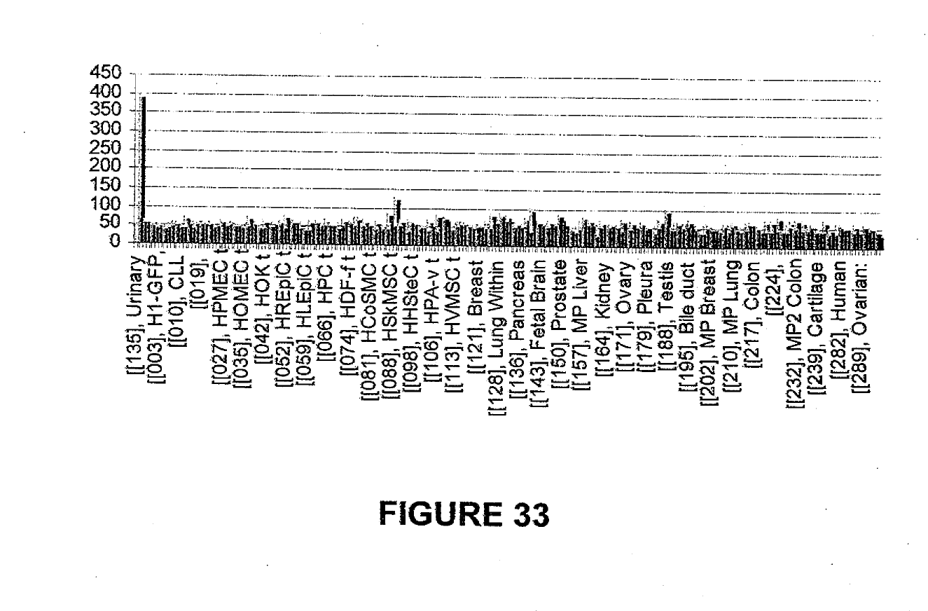

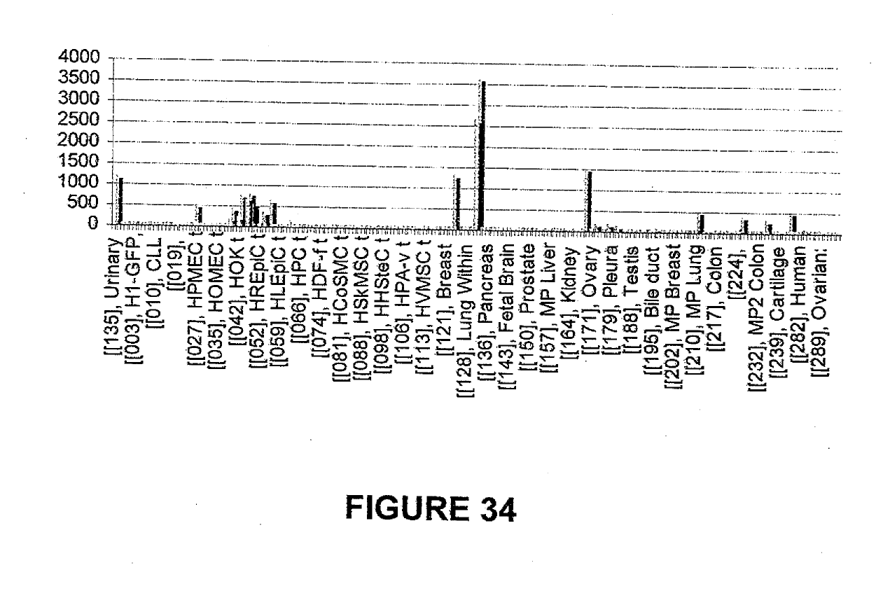

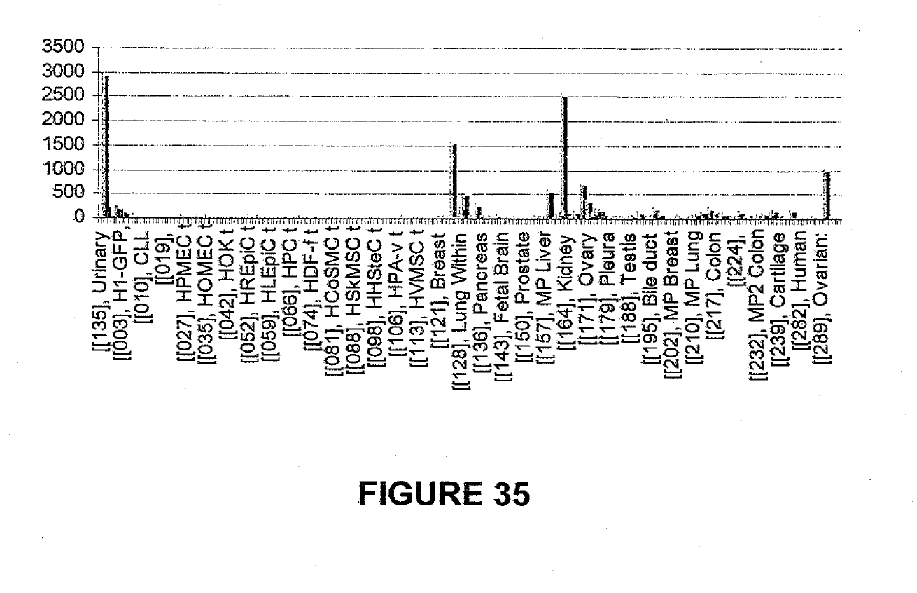

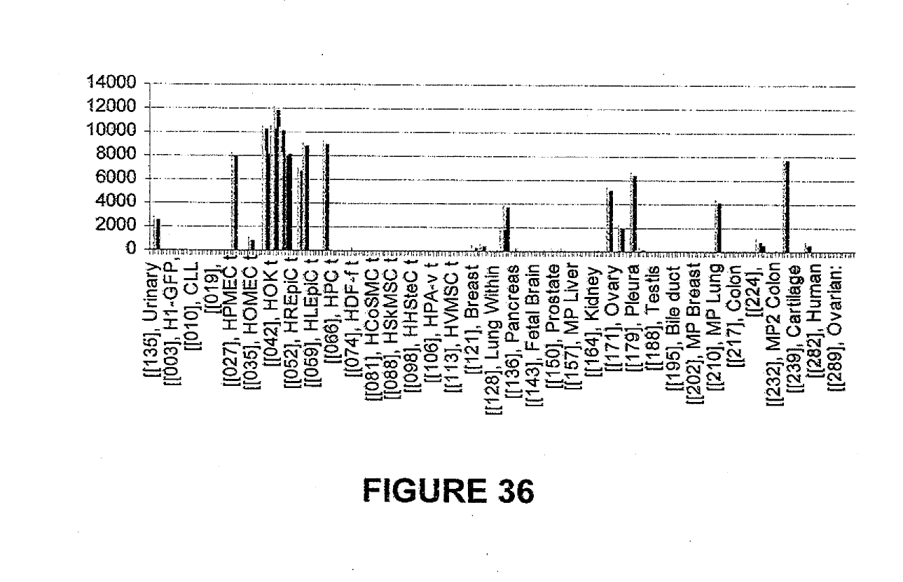

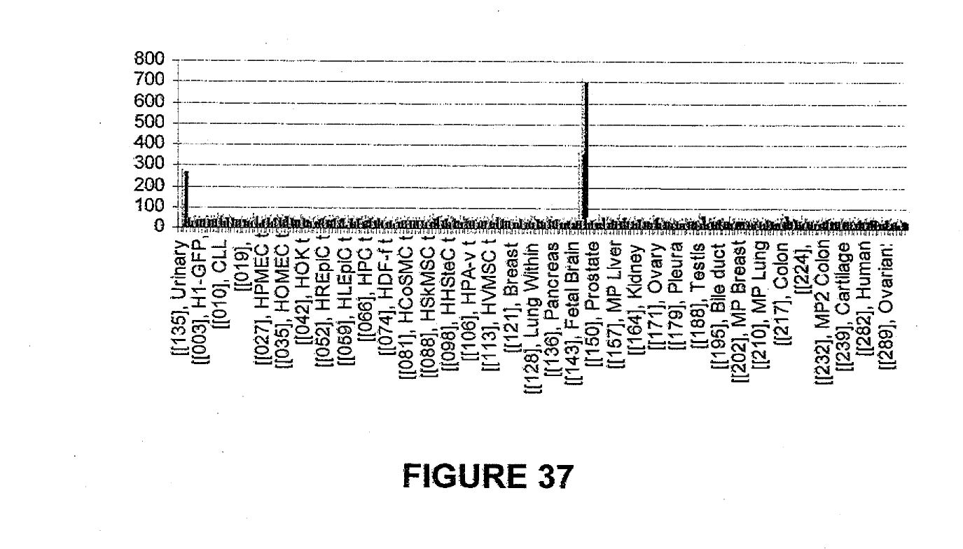

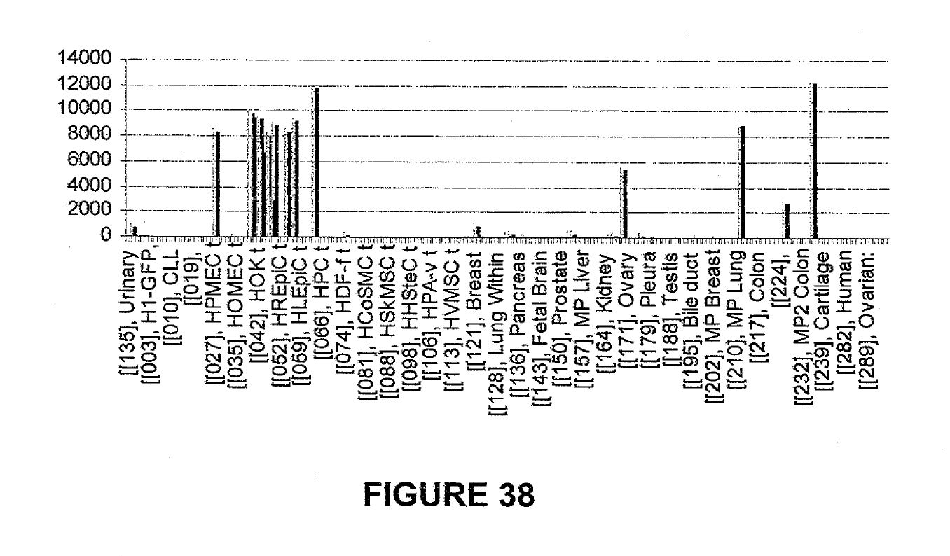

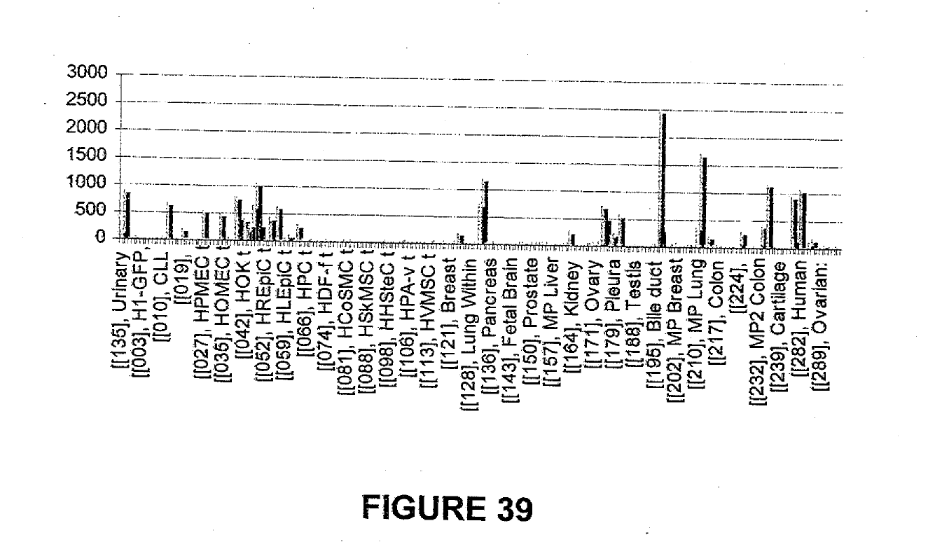

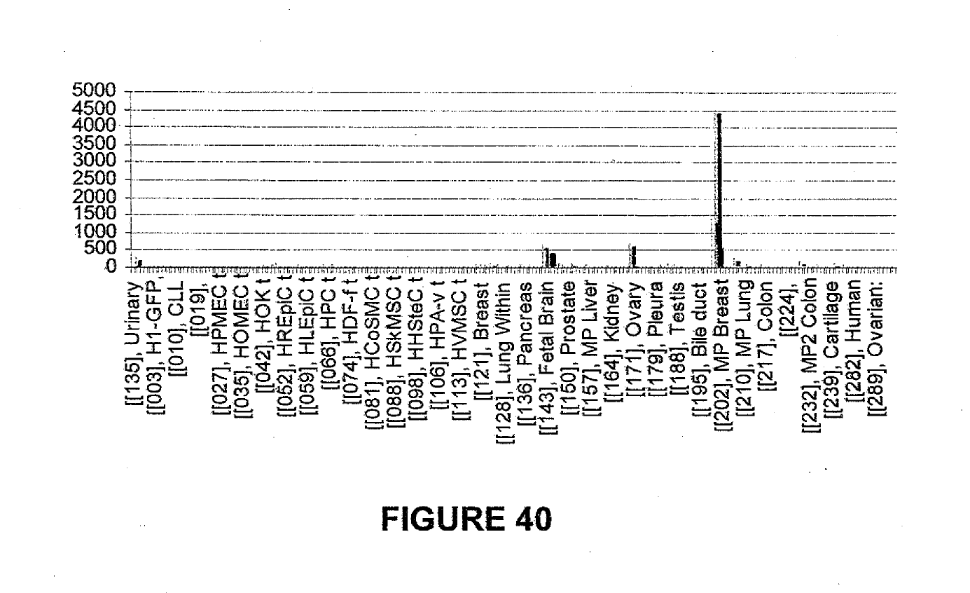

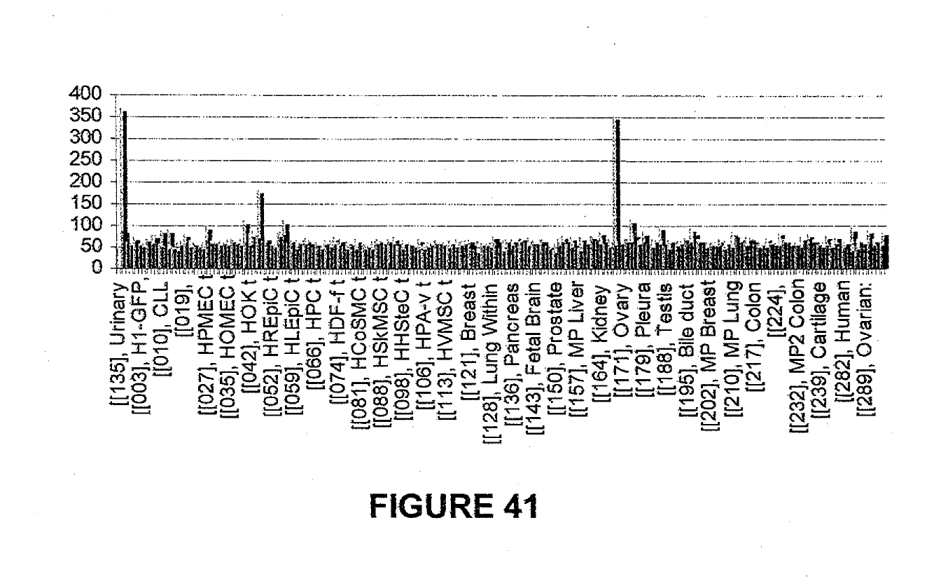

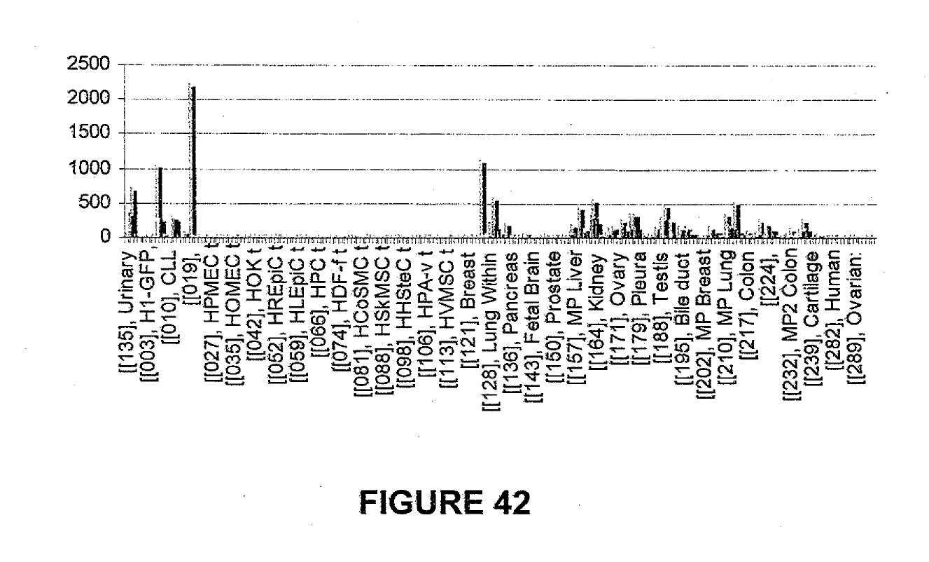

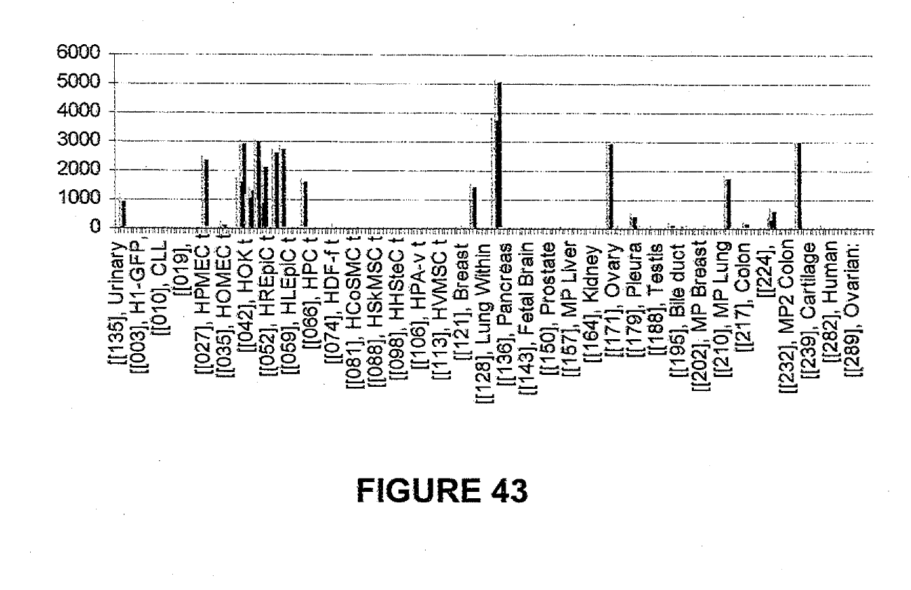

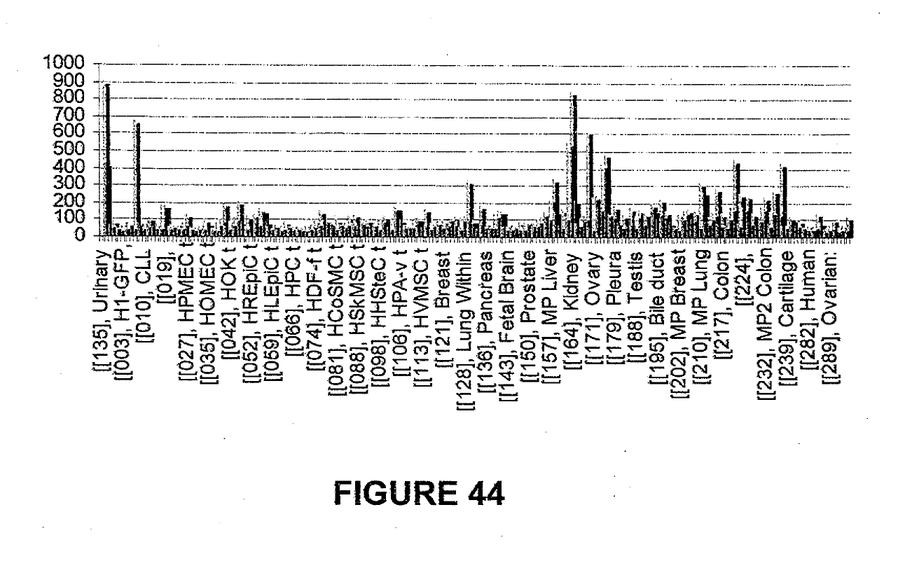

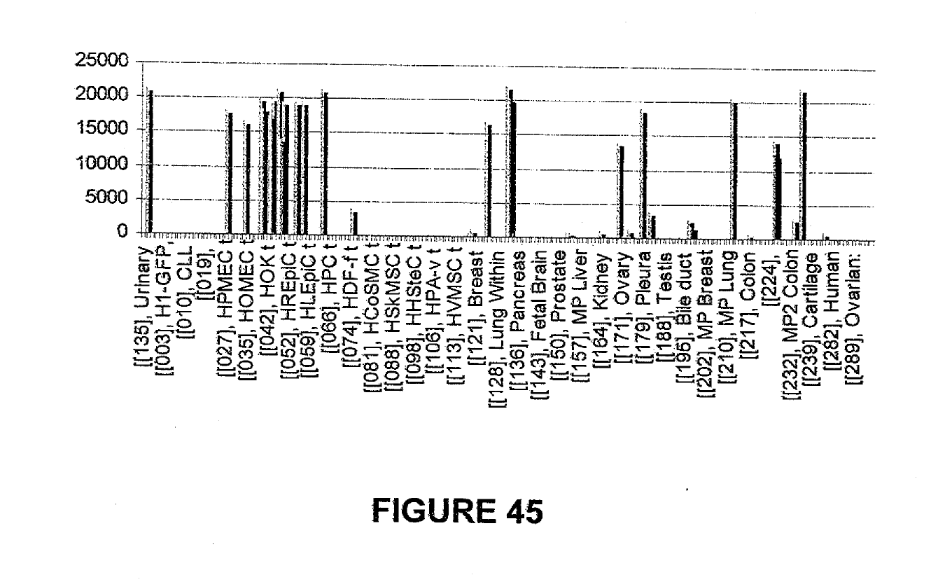

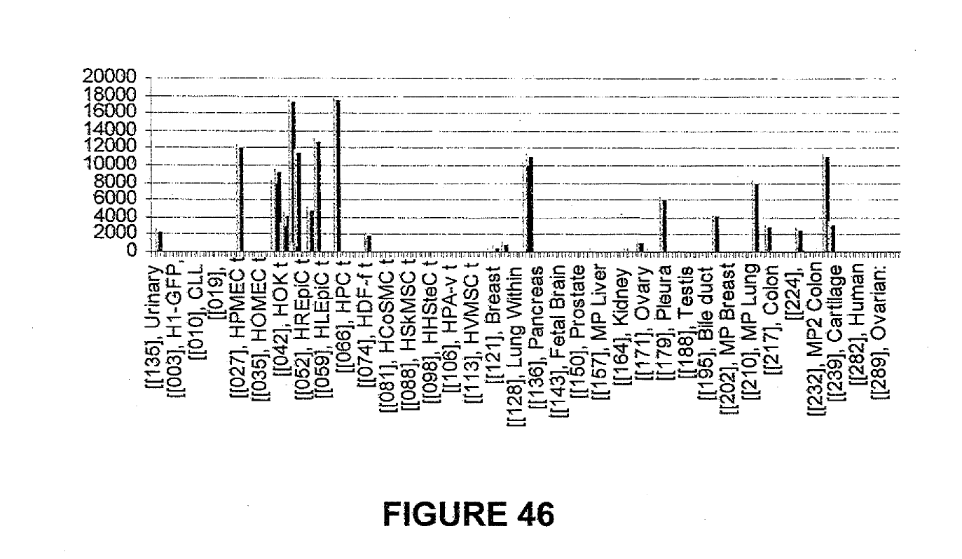

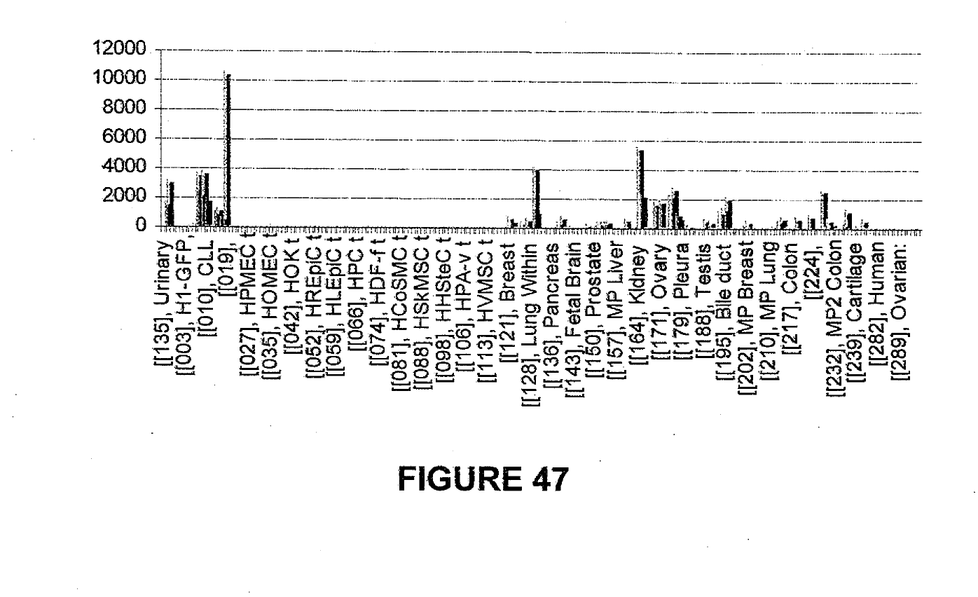

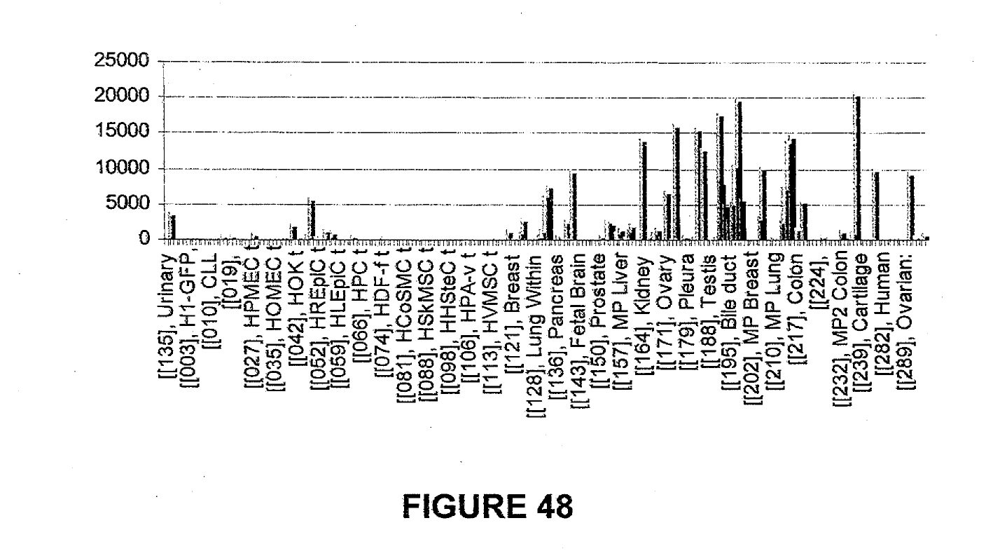

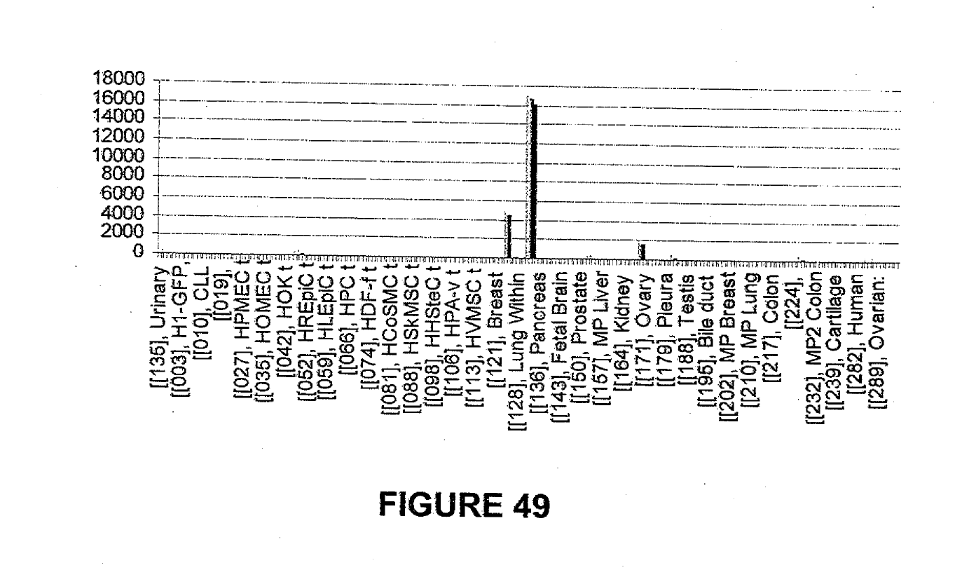

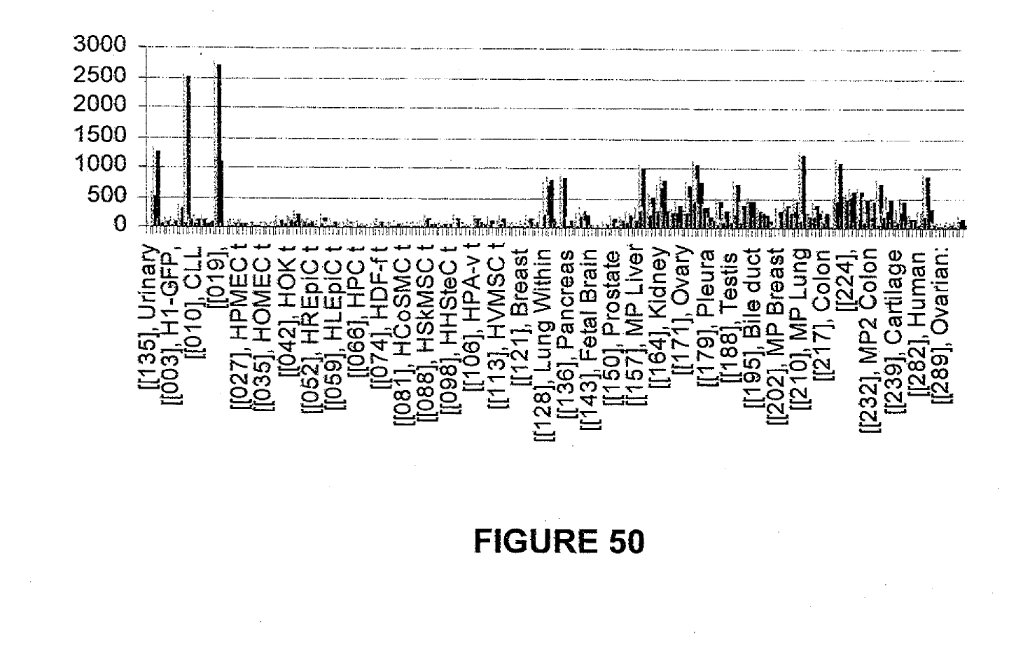

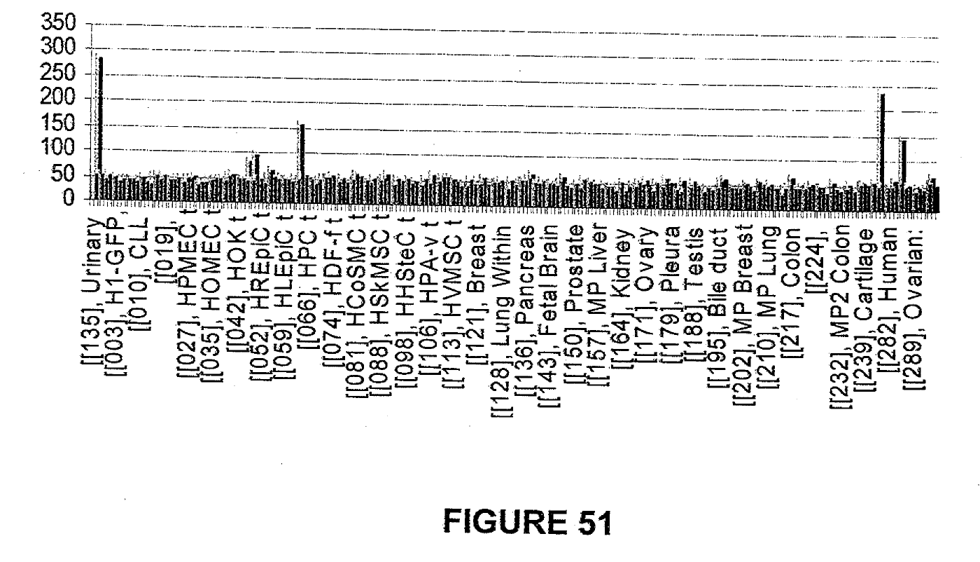

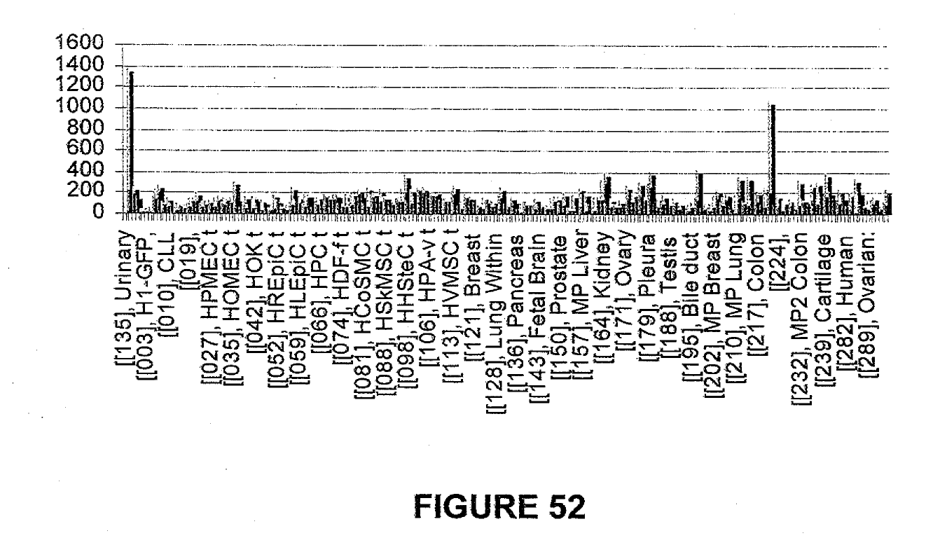

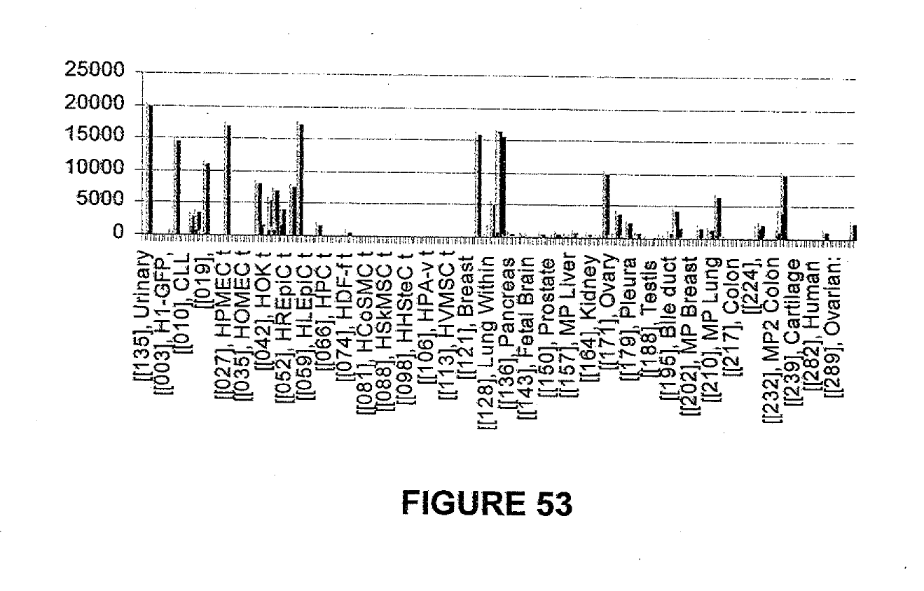

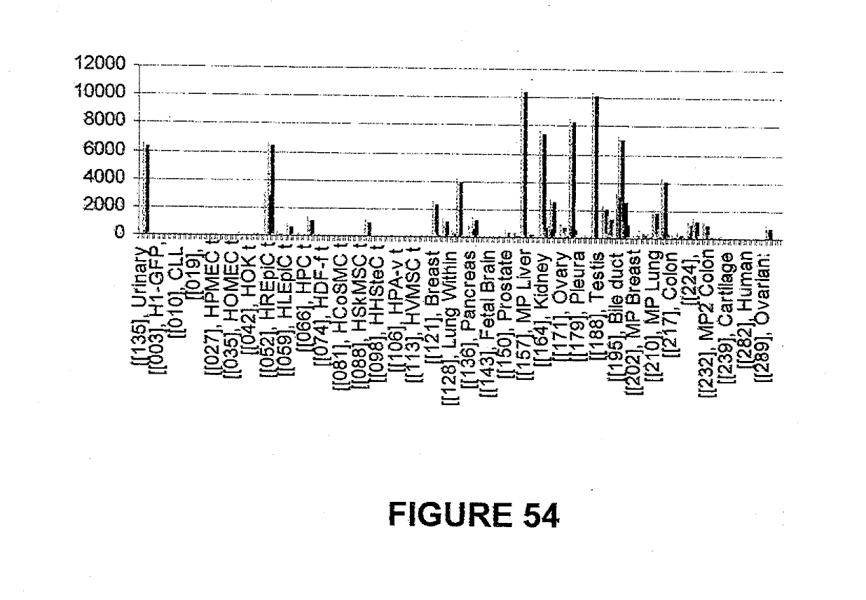

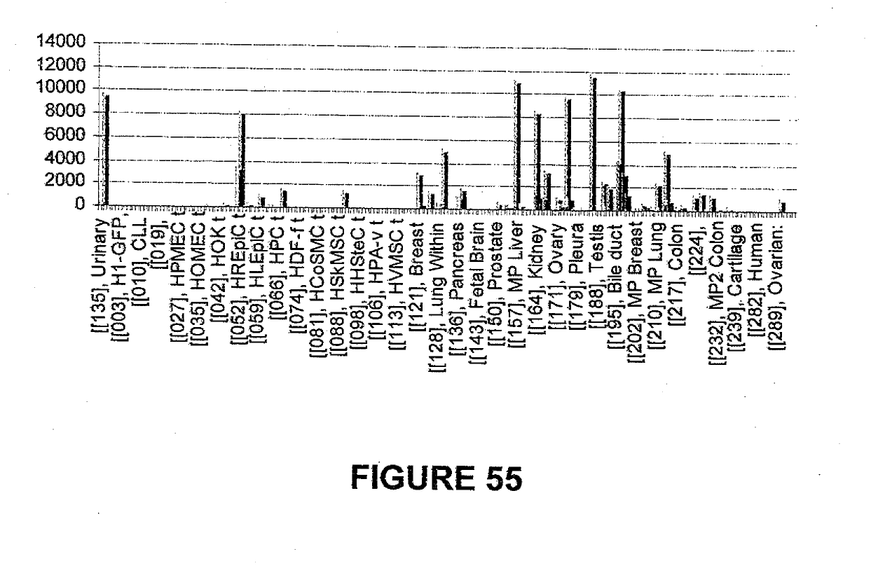

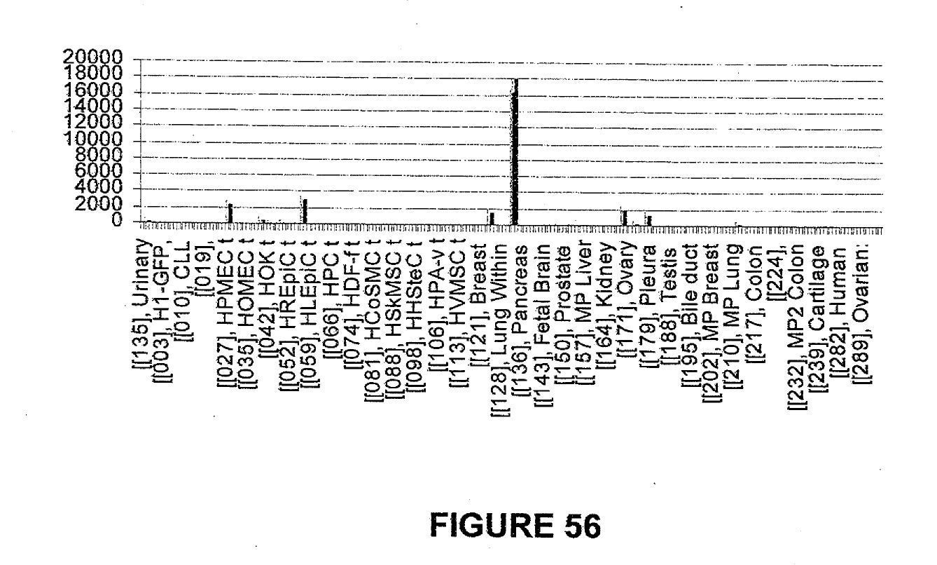

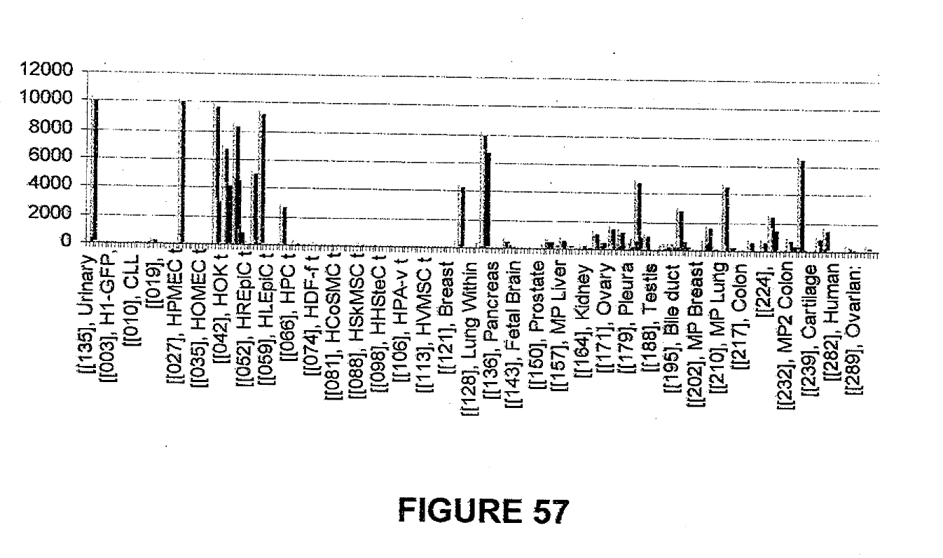

DSCR8 expression in diverse cancer types. RNA was obtained from cultured diverse cultured human cell types, normal human tissues, and malignant human tumors and analyzed on Illumina gene expression microarrays. The gene encoding the protein down syndrome critical region gene 8 DSCR8 also known as MMA-1a (Illumina Probe ID 4280132, accession number NM_{—}203428.1) was detected as a gene expressed in relatively higher levels in testis and diverse cancers compared to normal cultured somatic cell types and tissues. There are reports that DSCR8 is expressed in testis and in melanoma (de Wit, N. J. et al Expression profiling of MMA-1a and splice variant MMA-1b: new cancer/testis antigens identified in human melanoma. Int. J. Cancer 98:547-553) and uterine (Risinger, J. I. et al (2007) Global expression analysis of cancer/testis genes in uterine cancers reveals a high incidence of BORIS expression. Clin. Cancer Res. 13:1713-1719) cancer. Measurements of DSCR8 may be useful for screening or diagnosing a wide array of cancers. While these previous reports suggest DSCR8 is expressed in relatively specifically in testis compared to other human tissues and report that it is expressed in uterine cancers and melanomas, they do not report that the relative expression of DSCR8 is diagnostic of the malignant tumors described herein. Surprisingly, as shown in FIG. 18, while diverse cultured normal somatic cell types such as brain microvascular endothelial cells, dermal fibroblasts, smooth muscle cells, esophageal epithelial cells, urothelial cells, pulmonary epithelial cells, prostate epithelial cells, hepatocytes, astrocytes, as well as others and normal tissues tested express relatively low levels of signal (i.e. either background signal of <100 RFU or in the case of eye-derived cells low (<250 RFUs)), samples of normal testis, and diverse malignant tumors expressed the gene at relatively high levels (>250 RFU). Examples of such tumors are: endometrial adenocarcinoma (as predicted based on the art), small cell lung cancer, bladder carcinoma, seminoma of the testis, adenocarcinoma of the stomach, the myelogenous leukemia cell line K562, the ovarian cancer cell line OVCAR3, and the melanoma cell line G361 (as expected in the art). Since sensitive technologies exist to express to detect genes such as DSCR8, said nucleotide probes such as PCR primers or the oligonucleotide probe used in the microarray described herein (TCCCACTTGGCAGGGGCCGTCTTGTCCACTCGTTTCTGTAAACATGGGTG), (SEQ ID NO:190) as well as other detection techniques described herein including but not limited to the detection of the protein in tissue samples or blood using monoclonal or polyclonal antibodies, may be used in the unexpected manner described herein to screen for or to otherwise stage the wide array of cancers described above.

In addition, the specific expression of DSCR8 in varied malignancies may provide novel therapeutic strategies wherein the knockdown or inhibition of the activity of the protein encoded by DSCR8 or down-regulating the expression or translation of the gene may be used in reducing tumor mass and treating cancer.

### Example 6

qPCR was performed on bladder tumor tissue, normal bladder tissue and normal bladder tissue that was located adjacent to a bladder tumor. Positive controls were bladder tumors previously assayed by microarray.

Total RNA was extracted with the RNeasy Mini Kit (Qiagen) and cDNA generated using the SuperScript III reverse transcriptase in combination with random hexamer primers alone or in combination with oligo-dT primers (all reverse transcription components from Invitrogen/Life Technologies). PCRs were carried out on a 7900HT Sequence Detection System or a 7500 Real Time PCR System (Applied Biosystems/Life Technologies) utilizing SYBR Green or TaqMan chemistries. The primers used for the PCR reactions are listed in Tables 7 and 8, PCR parameters were: activation at 50° C. for 2 minutes; denature at 95° C. for 10 minutes; followed by 40-42 cycles of 95° C. for 15 seconds and 60° C. for 1 minute (72° C. for amplicons >than 120 bp) followed by dissociation at 95° C. for 15 seconds; 60° C. for 15 seconds, and 95° C. for 15 seconds.

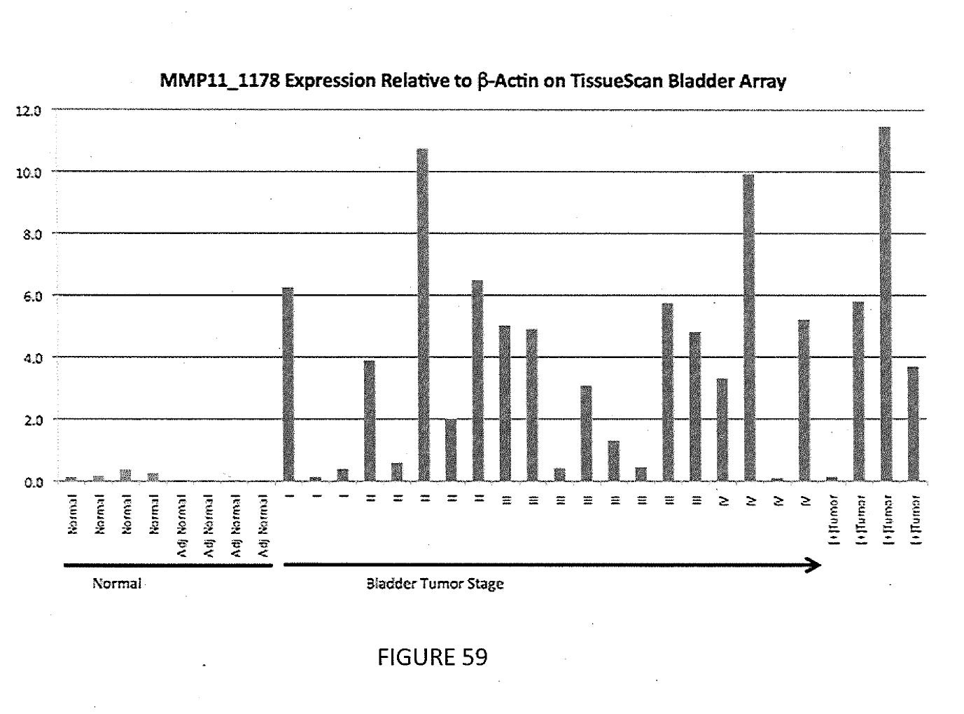

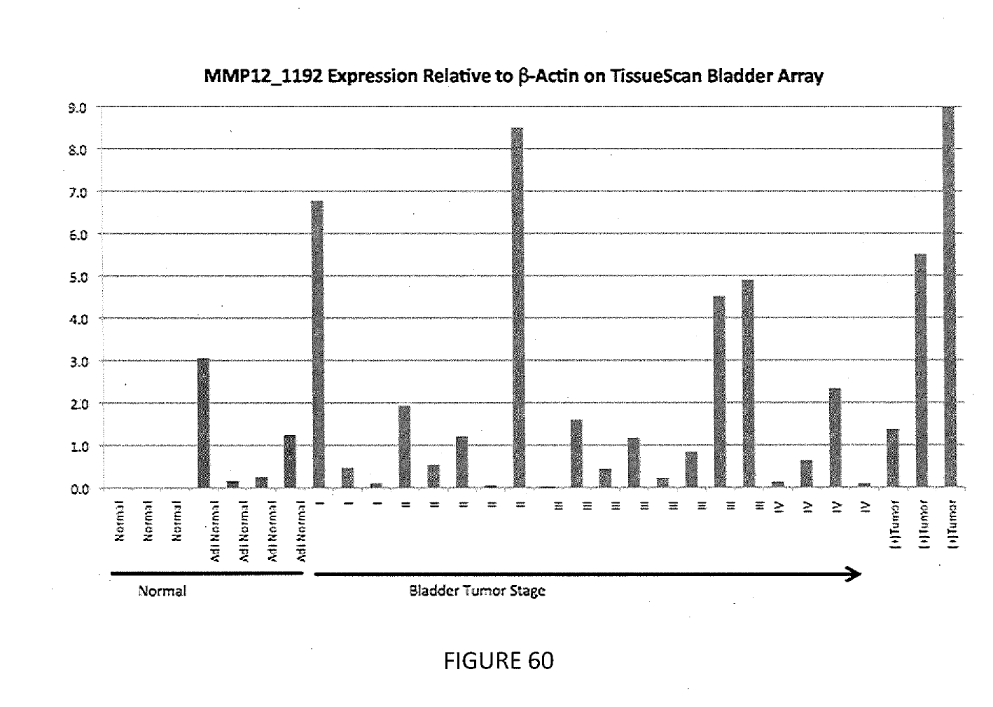

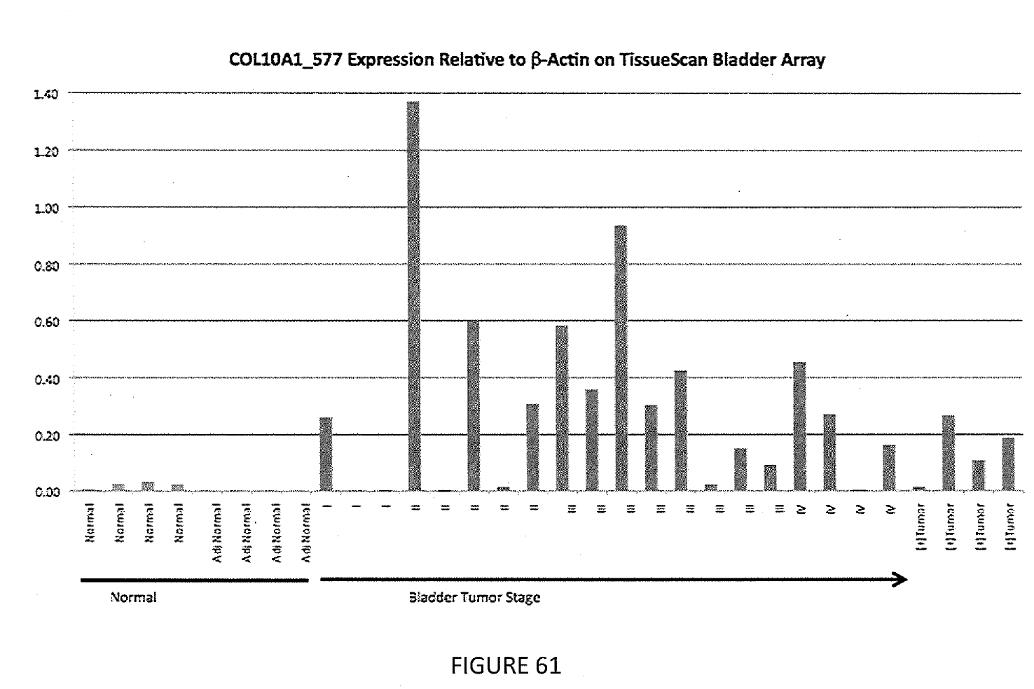

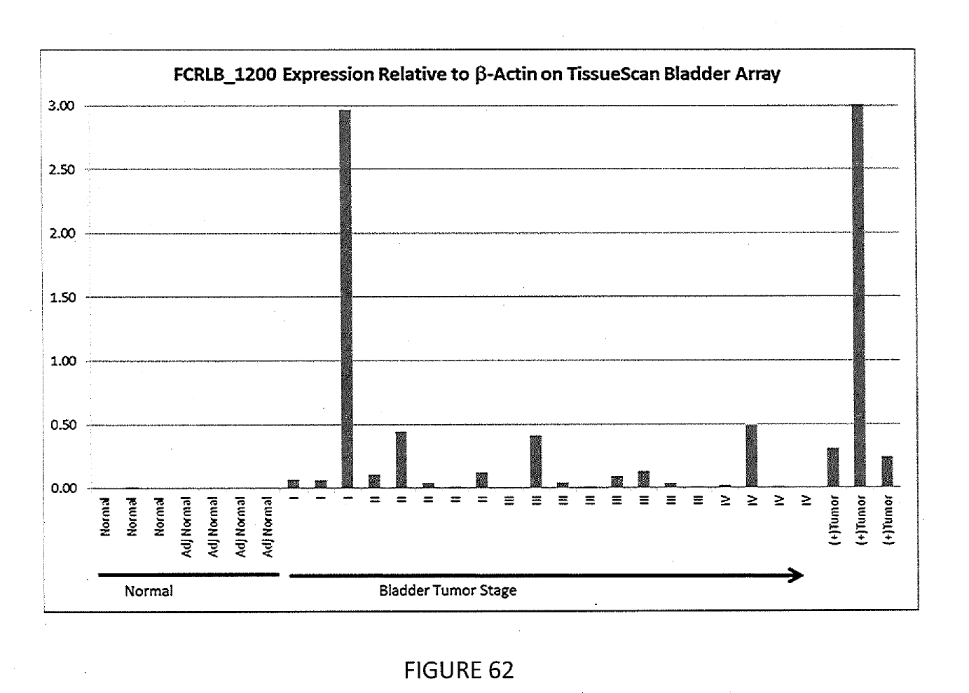

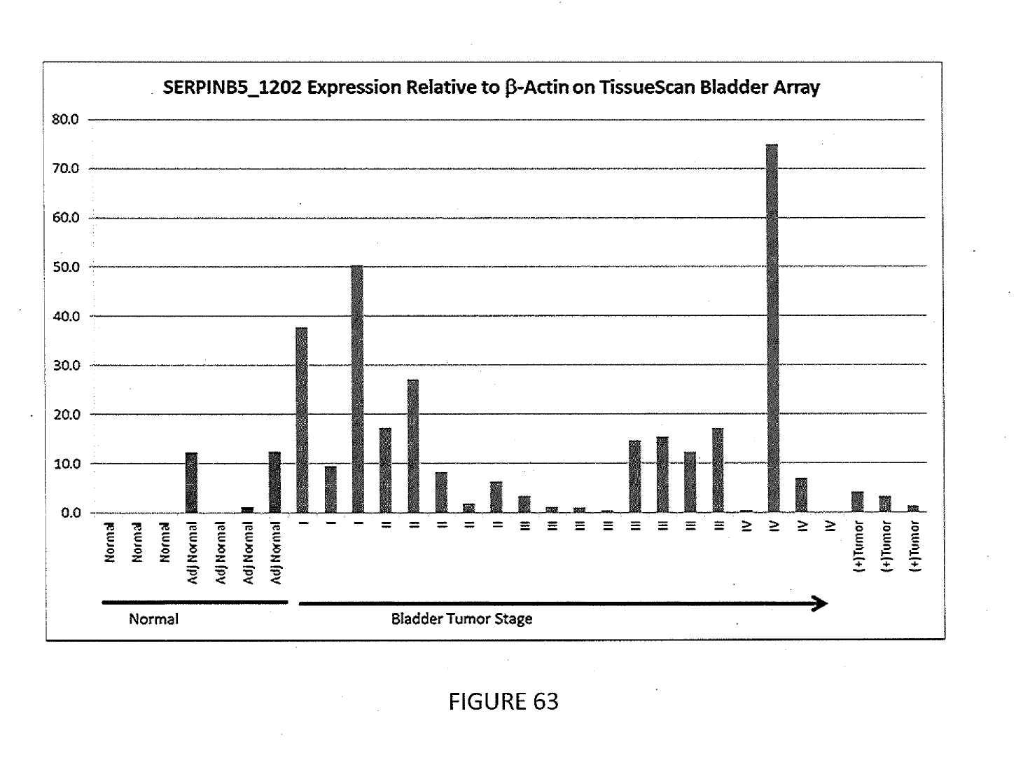

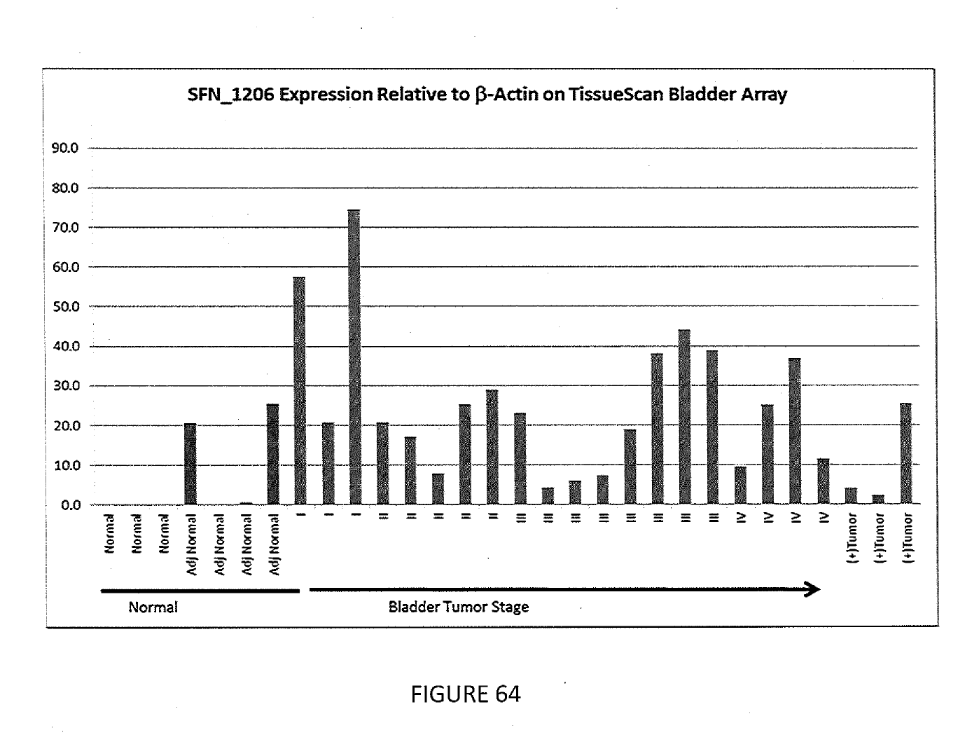

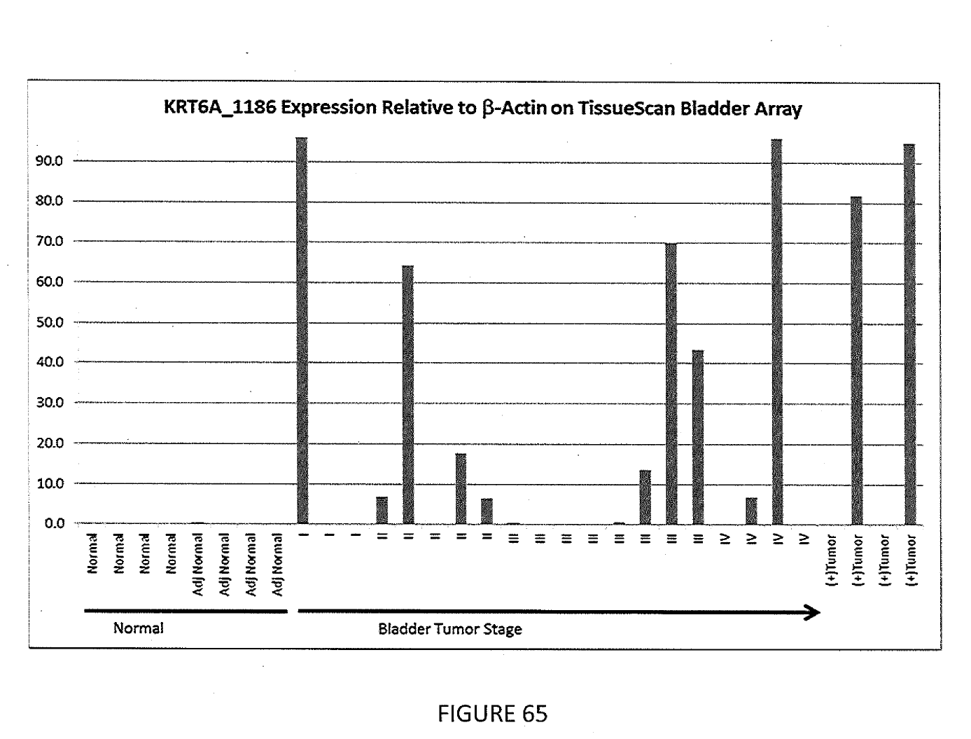

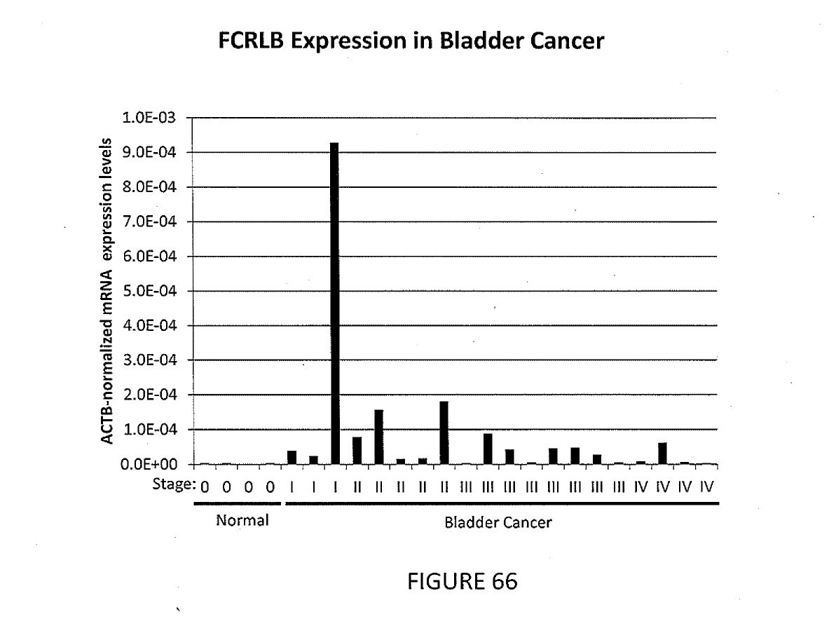

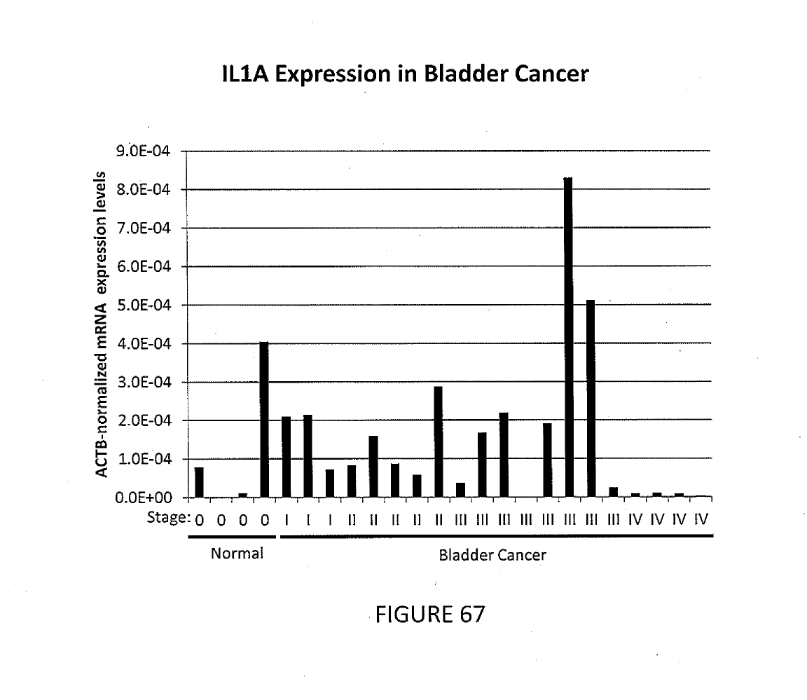

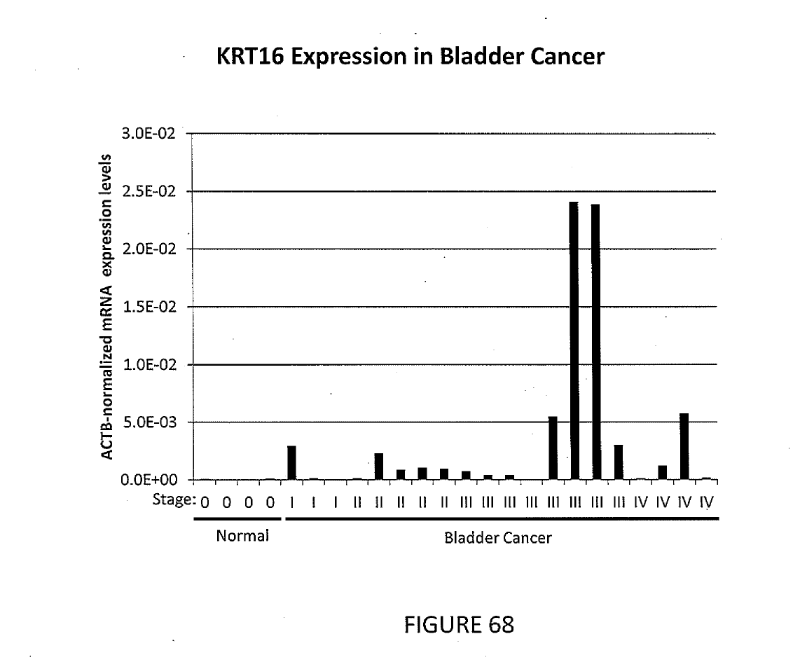

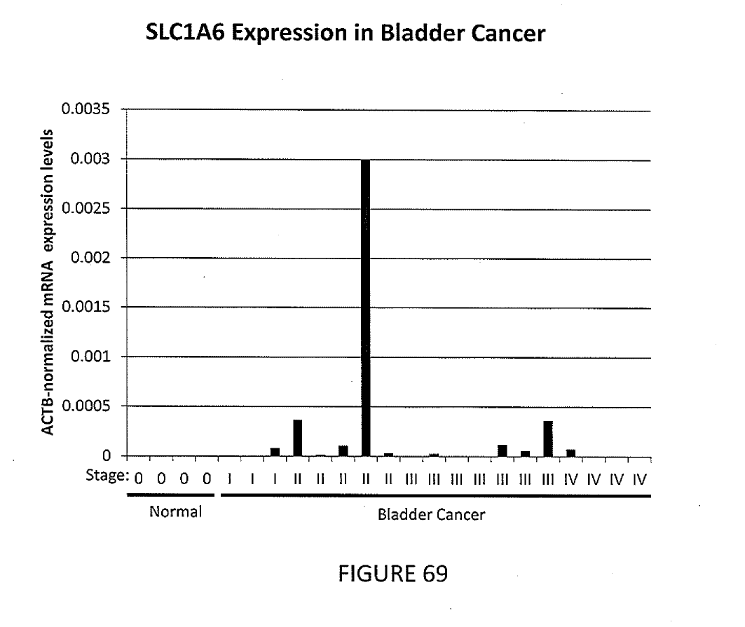

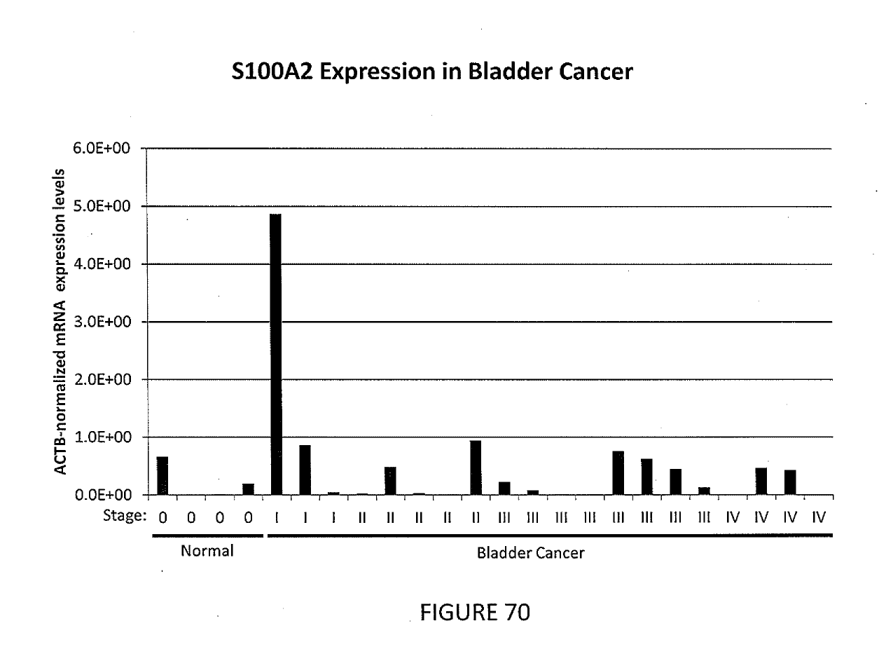

The results are provided in FIGS. 59-70 and showed that MMP-1, MMP-12, COL10A1, FCRLB, SERPINB5, SFN, KRT6A, FCRLB, IL1A, KRT16, SLC1A6, and S100A2 were all elevated relative to normal bladder tissue (normalized to β-actin expression). Moreover, the signal pattern seen for the positive controls previously analyzed by microarray, was the same obtained by microarray confirming that the PCR reaction worked.

### Example 7

The UPL System contains a relatively small number of short hydrolysis probes that cover an extensive proportion of the human mRNA transcriptome. UPL probes contain locked nucleic acids (LNAs) lowering the probes' melting temperatures. This allowed the probe and the longer, unmodified, primers to anneal at the same temperature.

Quantitative reverse transcription-polymerase chain reaction (qRT-PCR) was performed as follows:

Total RNA was extracted with the RNeasy Mini Kit (Qiagen) and cDNA generated using the SuperScript III reverse transcriptase in combination with random hexamer primers alone or in combination with oligo-dT primers (all reverse transcription components from Invitrogen/Life Technologies). PCRs were carried out on a 7900HT Sequence Detection System or a 7500 Real Time PCR System (Applied Biosystems/Life Technologies) utilizing SYBR® Green I (Applied Biosystems/Life Technologies) or TaqMan chemistries. TaqMan PCR was conducted with probes from the Universal Probe Library (UPL) (Roche) in combination with correspondingly designed primers. Primers: AAGCCTGCTGACGATGATG (Forward) (SEQ ID NO:191) and GCGAGGTAATGTATGCCCTTT (Reverse) (SEQ ID NO:192) were used with UPL 60. The results were normalized to β-actin expression levels.

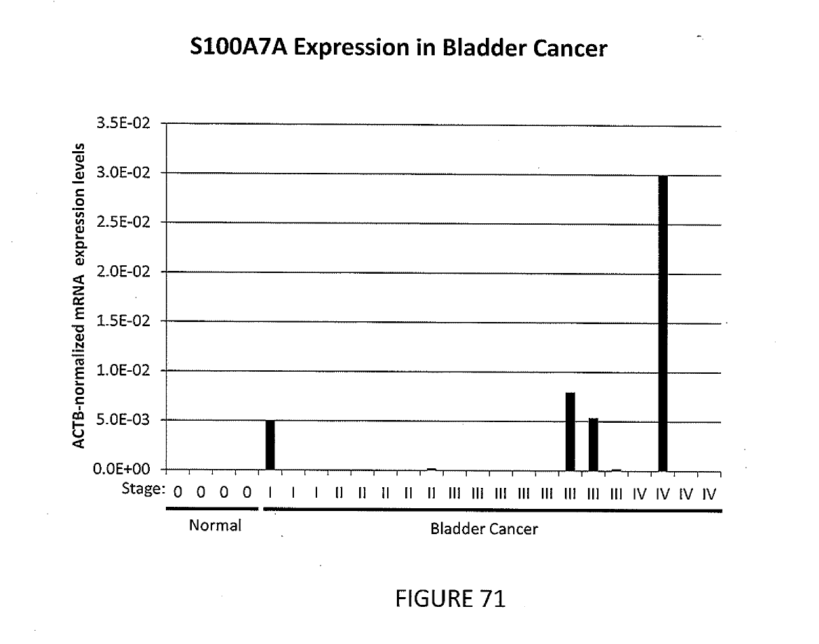

The result, indicating that S100A7A was elevated in bladder cancer, is shown in FIG. 71.

### Example 8

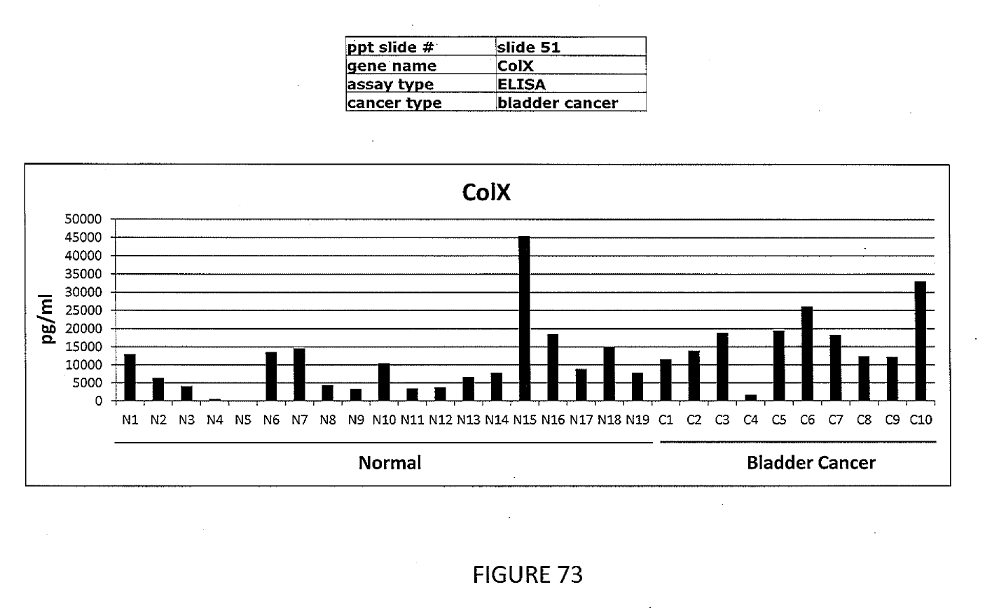

Example 8 provides ELISA data for MMP12, ColX and MMP11 (FIGS. 71-73).

Levels of the three protein markers were assayed in serum using a USCN ELISA kit (USCN) according to the manufacturer's instructions. In brief, 100 μL of the blank, standards, and samples with specified dilutions were added to the appropriate wells of a 96 well plate followed by 2 hours of incubation at 37° C. After removal of the liquid, 100 ul of Detection Reagent A was added to each well and incubated for 1 hour at 37° C. After removal of Reagent A, each well was washed 3 times with 350 uL of wash solution. 100 uL of Detection Reagent B was added to each well and then incubated for 30 minutes at 37° C. After removal of Reagent B, each well was washed 5 times with 350 uL of wash solution. 90 uL of Substrate solution was added to each well and incubated for 15-25 minutes at 37° C. 50 uL of Stop Solution was added to each well. The plate was read either on the Molecular Devices SpectraMax250 or the BioTek Synergy H1 plate reader at 450 nm. A standard curve was derived from the standards supplied in the kit and the sample values were extrapolated from this curve.

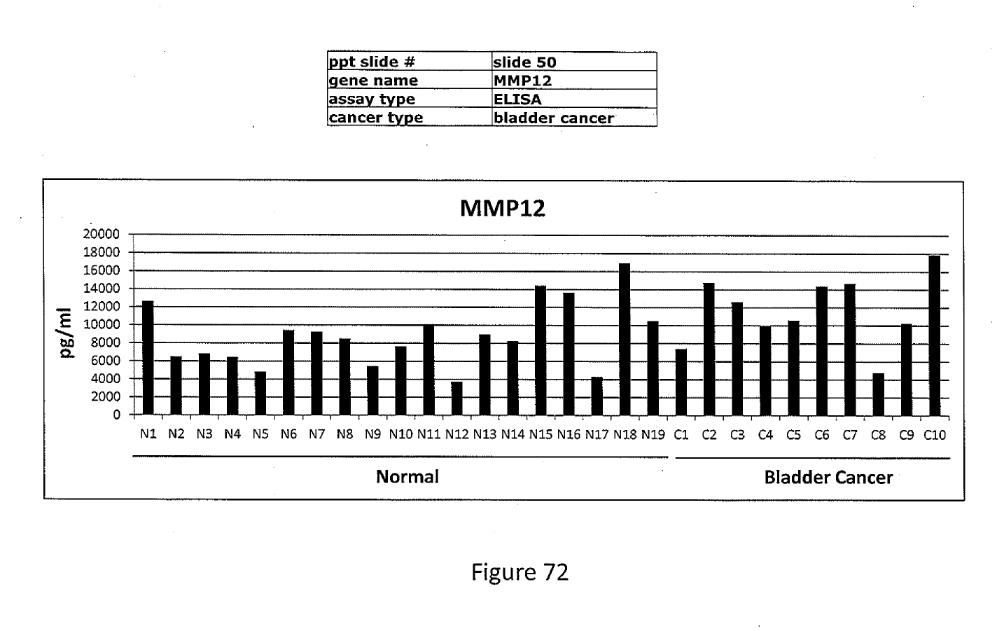

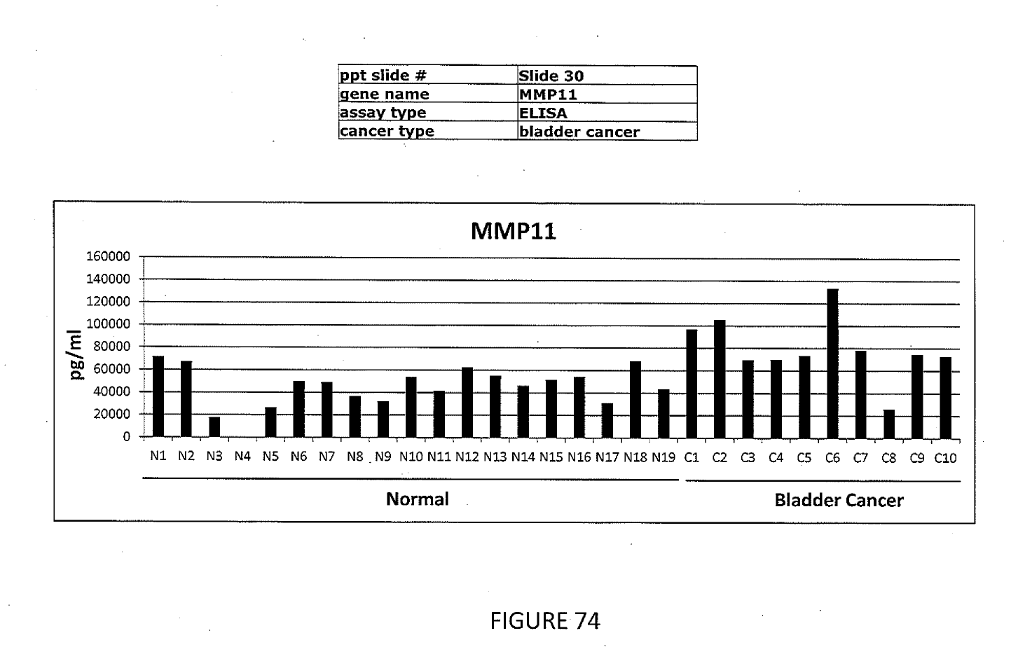

The results are shown in FIGS. 72-74 that MMP12, ColX and MMP11 are all elevated in bladder cancer samples.

### Example 9

Human urine samples from healthy subjects and cancer patients were analyzed by qPCR for expression of the markers COL10A1, MMP11, SFN, FCRLB, as described below.

RNA was extracted from cells in voided urine with the ZR Urine RNA Isolation Kit™ (Zymo Research) then reverse-transcribed using SuperScript III reverse transcriptase in the presence of random hexamer and oligo-dT primers (Invitrogen/Life Technologies). Following PCR with 50 cycles, products were analyzed on pre-cast 4% Agarose (HR) gels containing ethidium bromide (E-Gel®, Invitrogen/Life Technologies). Urine specimens: all from male individuals, three with bladder cancer (1-3), and three healthy controls (A-C). GAPDH served as loading and/or positive control. The following primers were used: COL10A1: ES577-COL10A1-F and ES578-COL10A1-R, MMP11: JK1178-MMP11-F and JK1179-MMP111-R, SFN: JK1206-SFN-F and JK1207-SFN-R, FCRLB: JK1200-FCRLB-F and JK1201-FCRLB-R, GAPDH: ES312-GAPD-F2 and ES313-GAPD-R2.

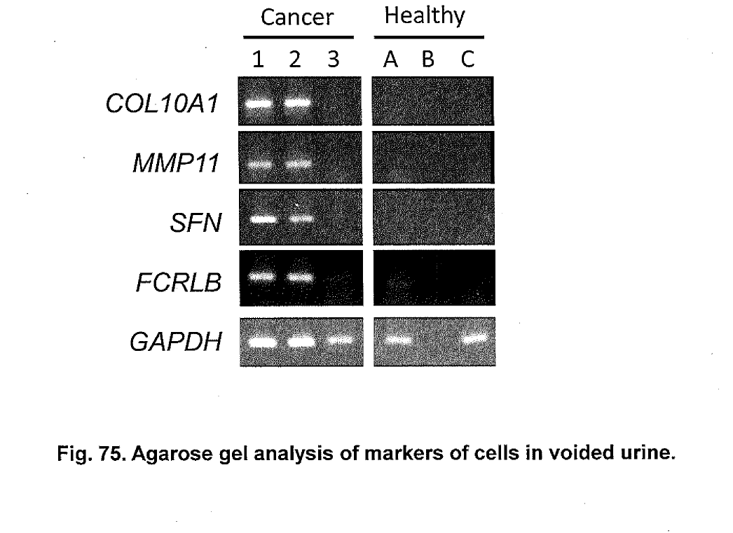

The results shown in FIG. 75 indicate that elevated levels of the markers COL10A1, MMP11, SFN, FCRLB are seen in the urine of cancer patients relative to healthy patients.

### Example 10

qPCR was performed on bladder tumor tissue, normal bladder tissue and normal bladder tissue that was located adjacent to a bladder tumor. Positive controls were bladder tumors previously assayed by microarray.

Total RNA was extracted with the RNeasy Mini Kit (Qiagen) and cDNA generated using the SuperScript III reverse transcriptase in combination with random hexamer primers alone or in combination with oligo-dT primers (all reverse transcription components from Invitrogen/Life Technologies). PCRs were carried out on a 7900HT Sequence Detection System or a 7500 Real Time PCR System (Applied Biosystems/Life Technologies) utilizing SYBR Green or TaqMan chemistries. The primers used for the PCR reactions were: ACTGGTGGCAGGGGCTTCTAGC (SEQ ID NO:196) (Forward primer) and GCCATCTAAAGTAACTAAACCCATAGAC (SEQ ID NO: 197) (REVERSE PRIMER). PCR parameters were: activation at 50° C. for 2 minutes; denature at 95° C. for 10 minutes; followed by 40-42 cycles of 95° C. for 15 seconds and 60° C. for 1 minute (72° C. for amplicons >than 120 bp) followed by dissociation at 95° C. for 15 seconds; 60° C. for 15 seconds, and 95° C. for 15 seconds.

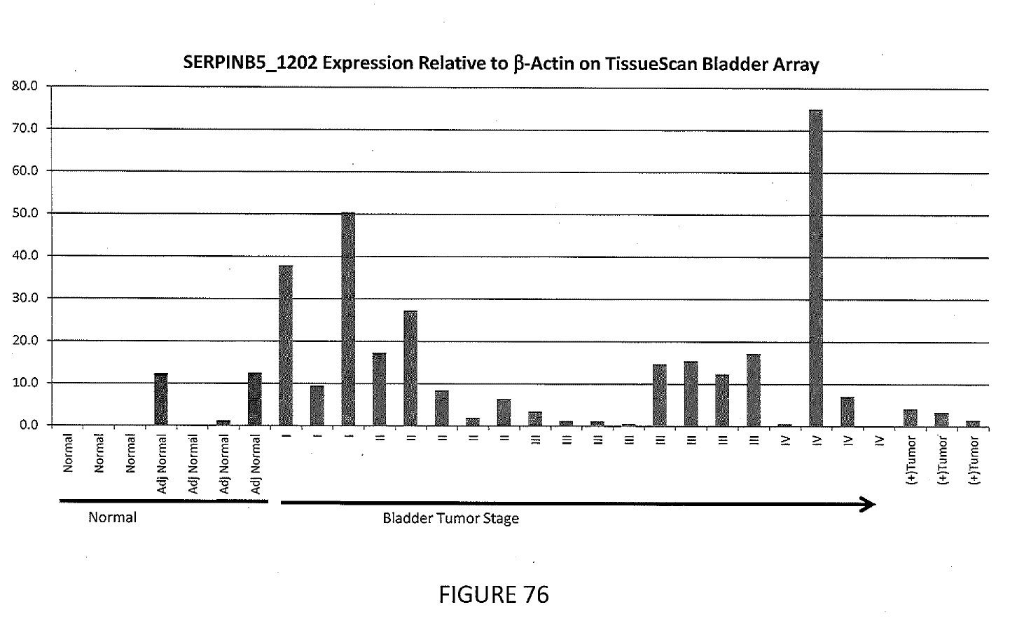

The results are provided in FIG. 76 and showed that SERPINB5 were all elevated relative to normal bladder tissue (normalized to β-actin expression). Moreover, the signal pattern seen for the positive controls previously analyzed by microarray, was the same obtained by microarray confirming that the PCR reaction worked.

Although the present invention has been described in considerable detail with reference to certain preferred embodiments thereof, other versions are possible. Therefore the spirit and scope of the appended claims should not be limited to the description and the preferred versions contained within this specification.

TABLE 5
SEQUENCES
Cancer-associated sequences disclosed in this application may
include the sequences disclosed below as well as any homologs,
complementary sequences, or combinations thereof.

Homo sapiens melanoma antigen family A, 10 (MAGEA10), transcript variant 2,
mRNA
NCBI Reference Sequence: NM_021048.3 (SEQ ID. NO. 1)

| | | |
|---|---|---|
| 1 | gagaagcgag gttctcgttc tgagggacag gcttgagatc ggctgaagag agcgggccca | |
| 61 | ggctctgtga ggaggcaagg gaggtgagaa ccttgctctc agagggtgac tcaagtcaac | 121 |
| | acagggaacc cctcttttct acagacacag tgggtcgcag gatctgacaa gagtccaggt | 181 |
| | tctcagggga cagggagagc aagaggtcaa gagctgtggg acaccacaga gcagcactga | 241 |
| | aggagaagac ctgcctgtgg gtccccatcg cccaagtcct gcccacactc ccacctgcta | 301 |
| | ccctgatcag agtcatcatg cctcgagctc caaagcgtca gcgctgcatg cctgaagaag | 361 |
| | atcttcaatc ccaaagtgag acacagggcc tcgagggtgc acaggctccc ctggctgtgg | 421 |
| | aggaggatgc ttcatcatcc acttccacca gctcctcttt tccatcctct tttccctcct | 481 |
| | cctcctcttc ctcctcctcc tcctgctatc ctctaatacc aagcacccca gaggaggttt | 541 |
| | ctgctgatga tgagacacca aatcctcccc agagtgctca gatagcctgc tcctccccct | 601 |
| | cggtcgttgc ttcccttcca ttagatcaat ctgatgaggg ctccagcagc caaaaggagg | 661 |
| | agagtccaag caccctacag gtcctgccag acagtgagtc tttacccaga agtgagatag | 721 |
| | atgaaaaggt gactgatttg gtgcagtttc tgctcttcaa gtatcaaatg aaggagccga | 781 |
| | tcacaaaggc agaaatactg gagagtgtca taaaaaatta tgaagaccac ttccctttgt | 841 |
| | tgtttagtga agcctccgag tgcatgctgc tggtctttgg cattgatgta aaggaagtgg | 901 |
| | atcccactgg ccactccttt gtccttgtca cctccctggg cctcacctat gatgggatgc | 961 |
| | tgagtgatgt ccagagcatg cccaagactg gcattctcat acttatccta agcataatct | 1021 |
| | tcatagaggg ctactgcacc cctgaggagg tcatctggga agcactgaat atgatggggc | 1081 |
| | tgtatgatgg gatggagcac ctcatttatg gggagcccag gaagctgctc acccaagatt | 1141 |
| | gggtgcagga aaactacctg gagtaccggc aggtgcctgg cagtgatcct gcacggtatg | 1201 |
| | agtttctgtg gggtccaagg gctcatgctg aaattaggaa gatgagtctc ctgaaatttt | 1261 |
| | tggccaaggt aaatgggagt gatccaagat ccttcccact gtggtatgag gaggctttga | 1321 |
| | aagatgagga agagagagcc caggacagaa ttgccaccac agatgatact actgccatgg | 1381 |
| | ccagtgcaag ttctagcgct acaggtagct tctcctaccc tgaataaagt aagacagatt | 1441 |
| | cttcactgtg ttttaaaagg caagtcaaat accacatgat tttactcata tgtggaatct | 1501 |
| | aaaaaaaaaa aaaaaaaaa | |

Homo sapiens Down syndrome critical region gene 8 (DSCR8), transcript variant
2, mRNA
NCBI Reference Sequence: NM_203428.1 (SEQ ID. NO. 2)

| | | |
|---|---|---|
| 1 | accccaccct aatcttgtta tgcaaatagg cttcccactt ggcaggggcc gtcttgtcca | |
| 61 | ctcgtttctg taaacatggg tggcaaaaag agaagatgga gctgccattt agaacatgcc | 121 |
| | taatcccagc ttcatcttgc tgagcaaaaa tgaaggagcc tggacccaac tttgttactg | 181 |
| | tgagaaaggg tcttcattca ttcaagatgg catttgttaa gcacctacta caaaccttgg | 241 |
| | aaatcaagaa agttctggaa tgatgaagct gttcatgcca agaccgaaag tgctggccca | 301 |
| | gtatgagtcc attcagttca tgccgtgaca attttcttgg aactcctttt tattgttagt | 361 |
| | tctcacttgt ttccatattt agtgaatgta catttaattg caaagctgtc attaataaaa | 421 |
| | attcttatag tacctcaaaa a | |

Homo sapiens matrix metallopeptidase 12 (macrophage elastase) (MMP12), mRNA
NCBI Reference Sequence: NM_002426.2 (SEQ ID. NO. 3)

| | | |
|---|---|---|
| 1 | agaaaggaac acagtaaact gaattgatcc gtttagaagt ttacaatgaa gtttcttcta | |
| 61 | atactgctcc tgcaggccac tgcttctgga gctcttcccc tgaacagctc tacaagcctg | 121 |
| | gaaaaaaata atgtgctatt tggtgaaaga tacttagaaa aattttatgg ccttgagata | 181 |
| | aacaaacttc cagtgacaaa aatgaaatat agtggaaact taatgaagga aaaatctcaa | 241 |
| | gaaatgcagc acttcttggg tctgaaagtg accgggcaac tggacacatc taccctggag | 301 |
| | atgatgcacg cacctcgatg tggagtcccc gatgtccatc atttcaggga aatgccaggg | 361 |
| | gggcccgtat ggaggaaaca ttatatcacc tacagaatca ataattacac acctgacatg | 421 |
| | aaccgtgagg atgttgacta cgcaatccgg aaagctttcc aagtatggag taatgttacc | 481 |
| | cccttgaaat tcagcaagat taacacaggc atggctgaca ttttggtggt ttttgcccgt | 541 |
| | ggagctcatg gagacttcca tgcttttgat ggcaaaggtg gaatcctagc ccatgctttt | 601 |
| | ggacctggat ctggcattgg aggggatgca catttcgatg aggacgaatt ctggactaca | 661 |
| | cattcaggag gcacaaactt gttcctcact gctgttcacg agattggcca ttccttaggt | 721 |
| | cttggccatt ctagtgatcc aaaggccgta atgttcccca cctacaaata tgttgacatc | 781 |
| | aacacatttc gcctctctgc tgatgacata cgtggcattc agtccctgta tggagaccca | 841 |
| | aaagagaacc aacgattgcc aaatcctgac aattcagaac cagctctctg tgaccccaat | 901 |
| | ttgagttttg atgctgtcac taccgtggga aataagatct ttttcttcaa agacaggttc | 961 |
| | ttctggctga aggtttctga gagaccaaag accagtgtta atttaatttc ttccttatgg | 1021 |
| | ccaaccttgc catctggcat tgaagctgct tatgaaattg aagccagaaa tcaagttttt | 1081 |
| | ctttttaaag atgacaaata ctggttaatt agcaatttaa gaccagagcc aaattatccc | 1141 |
| | aagagcatac attcttttgg ttttcctaac tttgtgaaaa aaattgatgc agctgttttt | 1201 |
| | aacccacgtt tttataggac ctacttcttt gtagataacc agtattggag gtatgatgaa | 1261 |
| | aggagacaga tgatggaccc tggttatccc aaactgatta ccaagaactt ccaaggaatc | 1321 |
| | gggcctaaaa ttgatgcagt cttctactct aaaaacaaat actactattt cttccaagga | 1381 |
| | tctaaccaat ttgaatatga cttcctactc caacgtatca ccaaaacact gaaaagcaat | 1441 |
| | agctggtttg gttgttagaa atggtgtaat taatggtttt tgttagttca cttcagctta | 1501 |
| | ataagtattt attgcatatt tgctatgtcc tcagtgtacc actacttaga gatatgtatc | 1561 |
| | ataaaaataa aatctgtaaa ccataggtaa tgattatata aaatacataa tatttttcaa | 1621 |
| | ttttgaaaac tctaattgtc cattcttgct tgactctact attaagtttg aaaatagtta | 1681 |
| | ccttcaaagg ccaagagaat tctatttgaa gcatgctctg taagttgctt cctaacatcc | 1741 |
| | ttggactgag aaattatact tacttctggc ataactaaaa ttaagtatat atattttggc | 1801 |
| | tcaaataaaa ttgaaaaaaa aatca | |

Homo sapiens chemokine (C—X—C motif) ligand 9 (CXCL9), mRNA
NCBI Reference Sequence: NM_002416.1 (SEQ ID. NO. 4)

| | | |
|---|---|---|
| 1 | atccaataca ggagtgactt ggaactccat tctatcacta tgaagaaaag tggtgttctt | |
| 61 | ttcctcttgg gcatcatctt gctggttctg attggagtgc aaggaacccc agtagtgaga | 121 |
| | aagggtcgct gttcctgcat cagcaccaac caagggacta tccacctaca atccttgaaa | 181 |
| | gaccttaaac aatttgcccc aagcccttcc tgcgagaaaa ttgaaatcat tgctacactg | 241 |
| | aagaatggag ttcaaacatg tctaaaccca gattcagcag atgtgaagga actgattaaa | 301 |
| | aagtgggaga aacaggtcag ccaaaagaaa aagcaaaaga atgggaaaaa acatcaaaaa | 361 |
| | aagaaagttc tgaaagttcg aaaatctcaa cgttctcgtc aaaagaagac tacataagag | 421 |
| | accacttcac caataagtat tctgtgttaa aaatgttcta ttttaattat accgctatca | 481 |
| | ttccaaagga ggatggcata taatacaaag gcttattaat ttgactagaa aatttaaaac | 541 |
| | attactctga aattgtaact aaagttagaa agttgatttt aagaatccaa acgttaagaa | 601 |
| | ttgttaaagg ctatgattgt ctttgttctt ctaccaccca ccagttgaat ttcatcatgc | 661 |
| | ttaaggccat gattttagca atacccatgt ctacacagat gttcacccaa ccacatccca | 721 |
| | ctcacaacag ctgcctggaa gagcagccct aggcttccac gtactgcagc ctccagagag | 781 |
| | tatctgaggc acatgtcagc aagtcctaag cctgttagca tgctggtgag ccaagcagtt | 841 |
| | tgaaattgag ctggacctca ccaagctgct gtggccatca acctctgtat ttgaatcagc | 901 |
| | ctacaggcct cacacacaat gtgtctgaga gattcatgct gattgttatt gggtatcacc | 961 |
| | actggagatc accagtgtgt ggctttcaga gcctcctttc tggctttgga agccatgtga | 1021 |
| | ttccatcttg cccgctcagg ctgaccactt tatttctttt tgttcccctt tgcttcattc | 1081 |
| | aagtcagctc ttctccatcc taccacaatg cagtgccttt cttctctcca gtgcacctgt | 1141 |
| | catatgctct gatttatctg agtcaactcc tttctcatct tgtccccaac accccacaga | 1201 |
| | agtgctttct tctcccaatt catcctcact cagtccagct tagttcaagt cctgcctctt | 1261 |
| | aaataaacct ttttggacac acaaattatc ttaaaactcc tgtttcactt ggttcagtac | 1321 |
| | cacatgggtg aacactcaat ggttaactaa ttcttgggtg tttatcctat ctctccaacc | 1381 |
| | agattgtcag ctccttgagg gcaagagcca cagtatattt ccctgtttct tccacagtgc | 1441 |
| | ctaataatac tgtggaacta ggttttaata attttttaat tgatgttgtt atgggcagga | 1501 |
| | tggcaaccag accattgtct cagagcaggt gctggctctt tcctggctac tccatgttgg | 1561 |
| | ctagcctctg gtaacctctt acttattatc ttcaggacac tcactacagg gaccagggat | 1621 |
| | gatgcaacat ccttgtcttt ttatgacagg atgtttgctc agcttctcca acaataagaa | 1681 |
| | gcacgtggta aaacacttgc ggatattctg gactgttttt aaaaaatata cagtttaccg | 1741 |
| | aaaatcatat aatcttacaa tgaaaaggac tttatagatc agccagtgac caaccttttc | 1801 |
| | ccaaccatac aaaaattcct tttcccgaag gaaaagggct ttctcaataa gcctcagctt | 1861 |
| | tctaagatct aacaagatag ccaccgagat ccttatcgaa actcatttta ggcaaatatg | 1921 |
| | agttttattg tccgtttact tgtttcagag tttgtattgt gattatcaat taccacacca | 1981 |
| | tctcccatga agaaagggaa cggtgaagta ctaagcgcta gaggaagcag ccaagtcggt | 2041 |
| | tagtggaagc atgattggtg cccagttagc ctctgcagga tgtggaaacc tccttccagg | 2101 |
| | ggaggttcag tgaattgtgt aggagaggtt gtctgtggcc agaatttaaa cctatactca | 2161 |
| | ctttcccaaa ttgaatcact gctcacactg ctgatgattt agagtgctgt ccggtggaga | 2221 |
| | tcccacccga acgtcttatc taatcatgaa actccctagt tccttcatgt aacttccctg | 2281 |
| | aaaaatctaa gtgtttcata aatttgagag tctgtgaccc acttaccttg catctcacag | 2341 |
| | gtagacagta tataactaac aaccaaagac tacatattgt cactgacaca cacgttataa | 2401 |
| | tcatttatca tatatataca tacatgcata cactctcaaa gcaaataatt tttcacttca | 2461 |
| | aaacagtatt gacttgtata ccttgtaatt tgaaatattt tctttgttaa aatagaatgg | 2521 |
| | tatcaataaa tagaccatta atcag | |

Homo sapiens Down syndrome critical region gene 8 (DSCR8), transcript variant
3, mRNA. NM_203429.1 (SEQ ID. NO. 5)

| | | |
|---|---|---|
| 1 | accccaccct aatcttgtta tgcaaatagg cttcccactt ggcaggggcc gtcttgtcca | |
| 61 | ctcgtttctg taaacatggg tggcaaaaag agaagatgga gctgccattt agaacatgcc | 121 |
| | taatcccagc ttcatcttgc tgagcaaaaa tgaaggagcc tggacccaac tttgttactg | 181 |
| | tgagaaaggg tcttcattca ttcaagatgg catttgttaa gcacctactg tgagtagatg | 241 |
| | atctcctgtc aaagacagtt aacaaatcct cggaatattg cttcatgtac agttattgga | 301 |
| | gatgagtaac ttacattctc ttaattgtaa tggttccttg gaaagtcatc gtggaaaatg | 361 |
| | aaggctggct catacatttt cccagacagg aatttggctg ccaacaggga attctaaaca | 421 |
| | actaaaaact ccagatgatg aatgcacaac ataatgatgg ttaaattaaa aaaaaaaaag | 481 |
| | agcacgacaa accttggaaa tcaagaaagt tctggaatga tgaagctgtt catgccaaga | 541 |
| | ccgaaagtgc tggcccagta tgagtccatt cagttcatgc cgtgacaatt ttcttggaac | 601 |
| | tcctttttat tgttagttct cacttgtttc catatttagt gaatgtacat ttaattgcaa | 661 |
| | agctgtcatt aataaaaatt cttatagtac ctcaaaaa | |

Homo sapiens keratin 81 (KRT81), mRNA
NCBI Reference Sequence: NM_002281.2 (SEQ ID. NO. 6)

| | | |
|---|---|---|
| 1 | actccaggtc ccctatcctg tcctctgcaa cccaaacgtc caggaggatc atgacctgcg | |
| 61 | gatcaggatt tggtgggcgc gccttcagct gcatctcggc ctgcgggccg cgccccggcc | 121 |
| | gctgctgcat caccgccgcc ccctaccgtg gcatctcctg ctaccgcggc ctcaccgggg | 181 |
| | gcttcggcag ccacagcgtg tgcggaggct ttcgggccgg ctcctgcgga cgcagcttcg | 241 |
| | gctaccgctc cgggggcgtg tgcgggccca gtcccccatg catcaccacc gtgtcggtca | 301 |
| | acgagagcct cctcacgccc ctcaacctgg agatcgaccc caacgcgcag tgcgtgaagc | 361 |
| | aggaggagaa ggagcagatc aagtccctca acagcaggtt cgcggccttc atcgacaagg | 421 |
| | tgcgcttcct ggagcagcag aacaaactgc tggagacaaa gctgcagttc taccagaacc | 481 |
| | gcgagtgttg ccagagcaac ctggagcccc tgtttgaggg ctacatcgag actctgcggc | 541 |
| | gggaggccga gtgcgtggag gccgacagcg ggaggctggc ctcagagctt aaccacgtgc | 601 |
| | aggaggtgct ggagggctac aagaagaagt atgaggagga ggtttctctg agagcaacag | 661 |
| | ctgagaacga gtttgtggct ctgaagaagg atgtggactg cgcctacctc cgcaagtcag | 721 |
| | acctggaggc caacgtggag gccctgatcc aggagatcga cttcctgagg cggctgtatg | 781 |
| | aggaggagat ccgcattctc cagtcgcaca tctcagacac ctccgtggtt gtcaagctgg | 841 |
| | acaacagccg ggacctgaac atggactgca tcattgccga gattaaggca cagtatgacg | 901 |
| | acattgtcac ccgcagccgg gccgaggccg agtcctggta ccgcagcaag tgtgaggaga | 961 |
| | tgaaggccac ggtgatcagg cacggggaga ccctgcgccg caccaaggag gagatcaatg | 1021 |
| | agctgaaccg catgatccaa aggctgacgg ccgaggtgga gaatgccaag tgccagaact | 1081 |
| | ccaagctgga ggccgcggtg gctcagtctg agcagcaggg tgaggcagcc ctcagtgatg | 1141 |
| | cccgctgcaa gctggccgag ctggagggcg ccctgcagaa ggccaagcag gacatggcct | 1201 |
| | gcctgatcag ggagtaccag gaggtgatga actccaagct gggcctggac atcgagatcg | 1261 |
| | ccacctacag gcgcctgctg gagggcgagg agcagaggct atgtgaaggc attggggctg | 1321 |
| | tgaatgtctg tgtcagcagc tcccggggcg gggtcgtgtg cggggacctc tgcgtgtcag | 1381 |
| | gctcccggcc agtgactggc agtgtctgca gcgctccgtg caacgggaac gtggcggtga | 1441 |
| | gcaccggcct gtgtgcgccc tgcggccaat tgaacaccac ctgcggaggg ggttcctgcg | 1501 |
| | gcgtgggctc ctgtggtatc agctccctgg gtgtggggtc ttgcggcagc agctgccgga | 1561 |
| | aatgttaggc accccaactc aagtcccagg ccccaggcat ctttgcctgc cctgccttgc | 1621 |
| | ttggcccagt cagtcaggcg cctggagaag tgctcagcta cttctcctgc actttgaaag | 1681 |
| | acccctccca ctcctggcct cacatttctc tgtgtgatcc cccacttctg ggctctgcca | 1741 |
| | ccccacagtg ggaaaggcca ccctagaaag aagtccgctg gcacccatag gaaggggcct | 1801 |
| | caggagcagg aagggccagg accagaacct tgcccacggc aactgccttc ctgcctctcc | 1861 |
| | ccttcctcct ctgctcttga tctgtgtttc aataaattaa tgtagccaaa aaaaaaaaaa | 1921 |
| | aaaaa | |

PREDICTED: Homo sapiens hypothetical protein LOC729826 (LOC729826), mRNA
NCBI Reference Sequence: XM_001131447.1 (SEQ ID. NO. 7)

| | | |
|---|---|---|
| 1 | aagagtaaaa tgtctcttta tctggaactt acatgatgat ttttccaaca aaataatttc | |
| 61 | caaatagatc acatagaaaa tgtctctttt aatatacttt acagtagtaa aactataaca | 121 |
| | tctcaattgt tttttttaaa taaatttgaa taatggttta ggtcatttga taaatatcac | 181 |
| | cttgtaacta ataagatgaa acaaggctaa ataggaccaa ttaagcacgt gatttaaata | 241 |
| | tcgatatgta gtgagtaaaa gagtaatcca actaccagta gaagattact acatttagta | 301 |
| | ctataacaag tataacactg ttcctaaaaa aaagtgcttt cttatgttta agatttattt | 361 |
| | taatgtcaaa cacaaataat tagatcttta aatgaacaat ttgggagtta aatccattgc | 421 |
| | ttctgatttt tatagatttt atggtctagg aaatctatac tgtctgattt gatcccattt | 481 |
| | aactgtaaga tttttacaca tgttgcactc tactagctgg caggaaaatt attttaatcg | 541 |
| | actgaatgaa agtattattt catgtaaaag tttattatat caaggaaatg atttaggtca | 601 |
| | gaagctagaa tctatataaa gtcagctttt gaaaataaag acagacaaat ctttttttac | 661 |
| | attattataa aagagctaag ttgcaaacaa ctatccttga gaccagacca ttttttttta | 721 |
| | agctgaaatt ttctaataat tgcaatggca aaacaccatt tgcaatttct tccctcccac | 781 |
| | ctcccaggtg gttcaacaat tccacttcca aacagcattt cccatcagtt tttaaaagct | 841 |
| | acttacaaag tgttattcta ctaccacttt taaatacatc aagcacttcc aaatatctag | 901 |
| | aaagactaga tatttcatat aacttgtcca ccacatacac atcactgtta aataaaattg | 961 |
| | cacacacata acaatggtta tcatctgagg tatcttctaa atgtggccat tttggccttg | 1021 |
| | aatcattccc tcctcccttc cttctctgcc ttcaatccag tggacaagta caggcacatg | 1081 |
| | taatgcttag agatggtcga acaaattcct atgcaaaagt ctttacagaa gacaagtttt | 1141 |
| | cctatgaatt ttaacacaaa gcgtacaaaa tatgctaatt ttactacttt gtcatacact | 1201 |
| | ggcaacctct ttaacaacta gagactagat gttgaaaaat taggactatt tgtccattat | 1261 |
| | atatactata tacagagcaa aacaaaatgc acaaaacgta tagaaaaatg gtgtctgaaa | 1321 |
| | atgtccaagt atgaacacac tagtatatta cctcttgcaa tttcttccct cctacctcct | 1381 |
| | ctaaaccatt gaacaagtat acacattact atactgctca caaaggtggc ttcacaattc | 1441 |
| | aatttccaaa agcatttcct atgaatttta gcaaaaagat atttacaaag tggtatttta | 1501 |
| | ctacctatac atttaacata catcgggcac ttctaaacat ctagatagac tagatgtttc | 1561 |
| | aagtaaggag ttaatttgtc tactatgtat acagcagtct tgaataaact gcaaacatgt | 1621 |
| | aacaacagtt ataatttgaa agagtcttcc aaatgtgaac attctggcct agaacccttc | 1681 |
| | ccatcgccat caacccagaa gacatcaaat tttcagaaga caatctttcc taggacttgt | 1741 |
| | aaaacaaaat gtacaaaata tattagttta ctaactctac ttttgtcata cactggcaac | 1801 |
| | ctctttaaca tccagaaaga ctagatgttg tcaattagga ctcgtctgtc ctttatgtac | 1861 |
| | attatataca cagataagta aaacaaaatg cacagacata catcttgcct cgctgtaaac | 1921 |
| | aggatggcat agagctctct gcacctcccc ctcctctctc ctcccctgaa ccactgcaca | 1981 |
| | aacacaatga gtattactca acaggtgatt tgaccattcc ccccaaaaaa tatttcctat | 2041 |
| | gaattgtaac aaaaaggtat ttacaaaatg tgattttgct acctctaatt ttaacatatc | 2101 |
| | aggcacttca gaacatctaa aaagaagaga catttcaaaa aagcttagca ttgtcaacta | 2161 |
| | tatacacagt agtgaggaat aaaatgcaca caaaacaatg gatagaatat gaaaatgtct | 2221 |
| | tctaaatatg accagtctag catagaacct tcttctcttc cttctcaggt cttccagctc | 2281 |
| | catgtcatct aacccactta acaaacgtga acgtatcgct tccagaggcc gtcttaacaa | 2341 |
| | ttccatttcc aaaagtcatc tccagaagac atgtattttc tacgatttct tttaaacaaa | 2401 |
| | tgagaattta caagatgtgt aactttctaa ctcttttatc ataactcgac aacctctttc | 2461 |
| | catctagaag ggctagatgt gacaaatgtt ttctattaaa aggttggggt ggagttgaga | 2521 |
| | gcagcttttt catattatat acacaggcct tccataaacg gccagtaaat cttcccagag | 2581 |
| | ggtggtgggc atttccaact ggccaaacgt ggcctgtcat tctaccattt ctctcttccg | 2641 |
| | acagcaaagt ctggtagaat gaagaccaac cgcccgatgg ccgctaaccg ttccacccgt | 2701 |
| | cgttgttcgg gacttcgctc acctttcagg ccccttaagg cctttgtccg ttgtcgtcag | 2761 |
| | gactaggtag gtctcgccca atggcgacag agtggtcacc cgggaaccgg atctgcgcgg | 2821 |
| | ctccgtggcc gaaagaggcg gccaagcctg cttgcgtccc taggccgcct tccgggccgt | 2881 |
| | ccacgcctta atggcctccg ccgcgcggcg ttcgagcggc cgccatactt cccggcccac | 2941 |
| | cacgcccggc gccgcccaaa ggcgctgcgt cctggcggct ctgcgggggt ttcgtcgagg | 3001 |
| | cccagcaggc ttgggtcggg agacccgggt gccggcgggg gccgggctgg gagacgccac | 3061 |
| | ggccgccatc agtcaccgag gtggggtggg aaagagaggt tcgctgcggc ttcaaggtct | 3121 |
| | gagcacagcc agtgggcagc cacagcagag gcctccggtg tctgcagggc agagggctcg | 3181 |
| | gcctgtcccg aggcccccca gttcatccgc cggcccgggg ccagagggcc ctgaaggcgc | 3241 |
| | gggctgcgtt ctgcgtctct ccgcgatctc tgccggaccg gaactaagac cagaccattt | 3301 |
| | tcttctggag taa | |

Homo sapiens parathyroid hormone-like hormone (PTHLH), transcript variant 3,
mRNA
NCBI Reference Sequence: NM_198964.1 (SEQ ID. NO. 8)

| | | |
|---|---|---|
| 1 | ctggttcgca aagaagctga cttcagaggg ggaaactttc ttcttttagg aggcggttag | |
| 61 | ccctgttcca cgaacccagg agaactgctg gccagattaa ttagacattg ctatgggaga | 121 |
| | cgtgtaaaca cactacttat cattgatgca tatataaaac cattttattt tcgctattat | 181 |
| | ttcagaggaa gcgcctctga tttgtttctt ttttcccttt ttgctctttc tggctgtgtg | 241 |
| | gtttggagaa agcacagttg gagtagccgg ttgctaaata agtcccgagc gcgagcggag | 301 |
| | acgatgcagc ggagactggt tcagcagtgg agcgtcgcgg tgttcctgct gagctacgcg | 361 |
| | gtgccctcct gcgggcgctc ggtggagggt ctcagccgcc gcctcaaaag agctgtgtct | 421 |
| | gaacatcagc tcctccatga caaggggaag tccatccaag atttacggcg acgattcttc | 481 |
| | cttcaccatc tgatcgcaga aatccacaca gctgaaatca gagctacctc ggaggtgtcc | 541 |
| | cctaactcca agccctctcc caacacaaag aaccaccccg tccgatttgg gtctgatgat | 601 |
| | gagggcagat acctaactca ggaaactaac aaggtggaga cgtacaaaga gcagccgctc | 661 |
| | aagacacctg ggaagaaaaa gaaaggcaag cccgggaaac gcaaggagca ggaaaagaaa | 721 |
| | aaacggcgaa ctcgctctgc ctggttagac tctggagtga ctgggagtgg gctagaaggg | 781 |
| | gaccacctgt ctgacacctc cacaacgtcg ctggagctcg attcacggta acaggcttct | 841 |
| | ctggcccgta gcctcagcgg ggtgctctca gctgggtttt ggagcctccc ttctgccttg | 901 |
| | gcttggacaa acctagaatt ttctcccttt atgtatctct atcgattgtg tagcaattga | 961 |
| | cagagaataa ctcagaatat tgtctgcctt aaagcagtac ccccctacca cacacacccc | 1021 |
| | tgtcctccag caccatagag aggcgctaga gcccattcct ctttctccac cgtcacccaa | 1081 |
| | catcaatcct ttaccactct accaaataat ttcatattca agcttcagaa gctagtgacc | 1141 |
| | atcttcataa tttgctggag aagtgtgttt cttcccctta ctctcacacc tgggcaaact | 1201 |
| | ttcttcagtg tttttcattt cttacgttct ttcacttcaa gggagaatat agaagcattt | 1261 |
| | gatattatct acaaacactg cagaacagca tcatgtcata aacgattctg agccattcac | 1321 |
| | actttttatt taattaaatg tatttaatta aatctcaaat ttattttaat gtaaagaact | 1381 |
| | taaattatgt tttaaacaca tgccttaaat ttgtttaatt aaatttaact ctggtttcta | 1441 |
| | ccagctcata caaaataaat ggtttctgaa aatgtttaag tattaactta caaggatata | 1501 |
| | ggtttttctc atgtatcttt ttgttcattg gcaagatgaa ataatttttc tagggtaatg | 1561 |
| | ccgtaggaaa aataaaactt cacatttatg tggcttgttt atccttagct cacagattga | 1621 |
| | ggtaataatg acactcctag actttgggat caaataactt agggccaagt cttgggtctg | 1681 |
| | aatttattta agttcacaac ctagggcaag ttactctgcc tttctaagac tcacttacat | 1741 |
| | cttctgtgaa atataattgt accaacctca tagagtttgg tgtcaactaa atgagattat | 1801 |
| | atgtggacta aatatctgtc atatagtaaa cactcaataa attgcaacat attaaaaaaa | 1861 |
| | aa | |

Homo sapiens matrix metallopeptidase 11 (stromelysin 3) (MPH), mRNA
NCBI Reference Sequence: NM_005940.3 (SEQ ID. NO. 9)

| | | |
|---|---|---|
| 1 | aagcccagca gccccggggc ggatggctcc ggccgcctgg ctccgcagcg cggccgcgcg | |
| 61 | cgccctcctg cccccgatgc tgctgctgct gctccagccg ccgccgctgc tggcccgggc | 121 |
| | tctgccgccg gacgcccacc acctccatgc cgagaggagg gggccacagc cctggcatgc | 181 |
| | agccctgccc agtagcccgg cacctgcccc tgccacgcag gaagcccccc ggcctgccag | 241 |
| | cagcctcagg cctccccgct gtggcgtgcc cgacccatct gatgggctga gtgcccgcaa | 301 |
| | ccgacagaag aggttcgtgc tttctggcgg gcgctgggag aagacggacc tcacctacag | 361 |
| | gatccttcgg ttcccatggc agttggtgca ggagcaggtg cggcagacga tggcagaggc | 421 |
| | cctaaaggta tggagcgatg tgacgccact cacctttact gaggtgcacg agggccgtgc | 481 |
| | tgacatcatg atcgacttcg ccaggtactg gcatggggac gacctgccgt ttgatgggcc | 541 |
| | tgggggcatc ctggcccatg ccttcttccc caagactcac cgagaagggg atgtccactt | 601 |
| | cgactatgat gagacctgga ctatcgggga tgaccagggc acagacctgc tgcaggtggc | 661 |
| | agcccatgaa tttggccacg tgctggggct gcagcacaca acagcagcca aggccctgat | 721 |
| | gtccgccttc tacacctttc gctacccact gagtctcagc ccagatgact gcaggggcgt | 781 |
| | tcaacaccta tatggccagc cctggcccac tgtcacctcc aggaccccag ccctgggccc | 841 |
| | ccaggctggg atagacacca atgagattgc accgctggag ccagacgccc cgccagatgc | 901 |
| | ctgtgaggcc tcctttgacg cggtctccac catccgaggc gagctctttt tcttcaaagc | 961 |
| | gggctttgtg tggcgcctcc gtgggggcca gctgcagccc ggctacccag cattggcctc | 1021 |
| | tcgccactgg cagggactgc ccagccctgt ggacgctgcc ttcgaggatg cccagggcca | 1081 |
| | catttggttc ttccaaggtg ctcagtactg ggtgtacgac ggtgaaaagc cagtcctggg | 1141 |
| | ccccgcaccc ctcaccgagc tgggcctggt gaggttcccg gtccatgctg ccttggtctg | 1201 |
| | gggtcccgag aagaacaaga tctacttctt ccgaggcagg gactactggc gtttccaccc | 1261 |
| | cagcacccgg cgtgtagaca gtcccgtgcc ccgcagggcc actgactgga gaggggtgcc | 1321 |
| | ctctgagatc gacgctgcct tccaggatgc tgatggctat gcctacttcc tgcgcggccg | 1381 |
| | cctctactgg aagtttgacc ctgtgaaggt gaaggctctg gaaggcttcc cccgtctcgt | 1441 |
| | gggtcctgac ttctttggct gtgccgagcc tgccaacact ttcctctgac catggcttgg | 1501 |
| | atgccctcag gggtgctgac ccctgccagg ccacgaatat caggctagag acccatggcc | 1561 |
| | atctttgtgg ctgtgggcac caggcatggg actgagccca tgtctcctca gggggatggg | 1621 |
| | gtggggtaca accaccatga caactgccgg gagggccacg caggtcgtgg tcacctgcca | 1681 |
| | gcgactgtct cagactgggc agggaggctt tggcatgact taagaggaag ggcagtcttg | 1741 |
| | ggcccgctat gcaggtcctg gcaaacctgg ctgccctgtc tccatccctg tccctcaggg | 1801 |
| | tagcaccatg gcaggactgg gggaactgga gtgtccttgc tccatccctg ttgtgaggtt | 1861 |
| | ccttccaggg gctggcactg aagcaagggt gctggggccc catggccttc agccctggct | 1921 |
| | gagcaactgg gctgtagggc agggccactt cctgaggtca ggtcttggta ggtgcctgca | 1981 |
| | tctgtctgcc ttctggctga caatcctgga aatctgttct ccagaatcca ggccaaaaag | 2041 |
| | ttcacagtca aatggggagg ggtattcttc atgcaggaga ccccaggccc tggaggctgc | 2101 |
| | aacatacctc aatcctgtcc caggccggat cctcctgaag cccttttcgc agcactgcta | 2161 |
| | tcctccaaag ccattgtaaa tgtgtgtaca gtgtgtataa accttcttct tctttttttt | 2221 |
| | tttttaaact gaggattgtc attaaacaca gttgttttct aaaaaaaaaa aaaaaa | |

Homo sapiens S100 calcium binding protein A7 (S100A7), mRNA
NCBI Reference Sequence: NM_002963.3 (SEQ ID. NO. 10)

| | | |
|---|---|---|
| 1 | gtccaaacac acacatctca ctcatccttc tactcgtgac gcttcccagc tctggctttt | |
| 61 | tgaaagcaaa gatgagcaac actcaagctg agaggtccat aataggcatg atcgacatgt | 121 |
| | ttcacaaata caccagacgt gatgacaaga ttgagaagcc aagcctgctg acgatgatga | 181 |
| | aggagaactt ccccaacttc cttagtgcct gtgacaaaaa gggcacaaat tacctcgccg | 241 |
| | atgtctttga gaaaaaggac aagaatgagg ataagaagat tgatttttct gagtttctgt | 301 |
| | ccttgctggg agacatagcc acagactacc acaagcagag ccatggagca gcgccctgtt | 361 |
| | ccgggggcag ccagtgaccc agccccacca atgggcctcc agagacccca ggaacaataa | 421 |
| | aatgtcttct cccaccagaa aaaaaaaaaa | |

Homo sapiens WNT1 inducible signaling pathway protein 3 (WISP3), transcript
variant 1, mRNA
NCBI Reference Sequence: NM_003880.2 (SEQ ID. NO. 11)

| | | |
|---|---|---|
| 1 | cctgagtccc gggaggaaag tgctcgccca ttcctgacct gtgacacgct cactgcgaag | |
| 61 | gcaggttatt agaagagtcc catgaaaggt ggctccacgg tcccagcgac atgcaggggc | 121 |
| | tcctcttctc cactcttctg cttgctggcc tggcacagtt ctgctgcagg gtacagggca | 181 |
| | ctggaccatt agatacaaca cctgaaggaa ggcctggaga agtgtcagat gcacctcagc | 241 |
| | gtaaacagtt ttgtcactgg ccctgcaaat gccctcagca gaagccccgt tgccctcctg | 301 |
| | gagtgagcct ggtgagagat ggctgtggat gctgtaaaat ctgtgccaag caaccagggg | 361 |
| | aaatctgcaa tgaagctgac ctctgtgacc cacacaaagg gctgtattgt gactactcag | 421 |
| | tagacaggcc taggtacgag actggagtgt gtgcatacct tgtagctgtt gggtgcgagt | 481 |
| | tcaaccaggt acattatcat aatggccaag tgtttcagcc caaccccttg ttcagctgcc | 541 |
| | tctgtgtgag tggggccatt ggatgcacac ctctgttcat accaaagctg gctggcagtc | 601 |
| | actgctctgg agctaaaggt ggaaagaagt ctgatcagtc aaactgtagc ctggaaccat | 661 |
| | tactacagca gctttcaaca agctacaaaa caatgccagc ttatagaaat ctcccactta | 721 |
| | tttggaaaaa aaaatgtctt gtgcaagcaa caaaatggac tccctgctcc agaacatgtg | 781 |
| | ggatgggaat atctaacagg gtgaccaatg aaaacagcaa ctgtgaaatg agaaaagaga | 841 |
| | aaagactgtg ttacattcag ccttgcgaca gcaatatatt aaagacaata aagattccca | 901 |
| | aaggaaaaac atgccaacct actttccaac tctccaaagc tgaaaaattt gtcttttctg | 961 |
| | gatgctcaag tactcagagt tacaaaccca ctttttgtgg aatatgcttg gataagagat | 1021 |
| | gctgtatccc taataagtct aaaatgatta ctattcaatt tgattgccca aatgaggggt | 1081 |
| | catttaaatg gaagatgctg tggattacat cttgtgtgtg tcagagaaac tgcagagaac | 1141 |
| | ctggagatat attttctgag ctcaagattc tgtaaaacca agcaaatggg ggaaaagtta | 1201 |
| | gtcaatcctg tcatataata aaaaaattag tgagtaaaaa aaaaaaaaaa aaaaaaaaaa | 1261 |
| | aaaaaaaaaa aaaaaaaaaa aaaaaagaaa aaaaaaaaaa aaaaaaa | |

Homo sapiens chemokine (C—X—C motif) ligand 10 (CXCL10), mRNA
NCBI Reference Sequence: NM_001565.2 (SEQ ID. NO. 12)

| | | |
|---|---|---|
| 1 | gggggagaca ttcctcaatt gcttagacat attctgagcc tacagcagag gaacctccag | |
| 61 | tctcagcacc atgaatcaaa ctgccattct gatttgctgc cttatctttc tgactctaag | 121 |
| | tggcattcaa ggagtacctc tctctagaac tgtacgctgt acctgcatca gcattagtaa | 181 |
| | tcaacctgtt aatccaaggt ctttagaaaa acttgaaatt attcctgcaa gccaattttg | 241 |
| | tccacgtgtt gagatcattg ctacaatgaa aaagaagggt gagaagagat gtctgaatcc | 301 |
| | agaatcgaag gccatcaaga atttactgaa agcagttagc aaggaaaggt ctaaaagatc | 361 |
| | tccttaaaac cagaggggag caaaatcgat gcagtgcttc caaggatgga ccacacagag | 421 |
| | gctgcctctc ccatcacttc cctacatgga gtatatgtca agccataatt gttcttagtt | 481 |
| | tgcagttaca ctaaaaggtg accaatgatg gtcaccaaat cagctgctac tactcctgta | 541 |
| | ggaaggttaa tgttcatcat cctaagctat tcagtaataa ctctaccctg gcactataat | 601 |
| | gtaagctcta ctgaggtgct atgttcttag tggatgttct gaccctgctt caaatatttc | 661 |
| | cctcaccttt cccatcttcc aagggtacta aggaatcttt ctgctttggg gtttatcaga | 721 |
| | attctcagaa tctcaaataa ctaaaaggta tgcaatcaaa tctgcttttt aaagaatgct | 781 |
| | ctttacttca tggacttcca ctgccatcct cccaaggggc ccaaattctt tcagtggcta | 841 |
| | cctacataca attccaaaca catacaggaa ggtagaaata tctgaaaatg tatgtgtaag | 901 |
| | tattcttatt taatgaaaga ctgtacaaag tagaagtctt agatgtatat atttcctata | 961 |
| | ttgttttcag tgtacatgga ataacatgta attaagtact atgtatcaat gagtaacagg | 1021 |
| | aaaattttaa aaatacagat agatatatgc tctgcatgtt acataagata aatgtgctga | 1081 |
| | atggttttca aaataaaaat gaggtactct cctggaaata ttaagaaaga ctatctaaat | 1141 |
| | gttgaaagat caaaaggtta ataaagtaat tataactaaa aaaa | |

Homo sapiens neuromedin U (NMU), mRNA
NCBI Reference Sequence: NM_006681.1 (SEQ ID. NO. 13)

| | | |
|---|---|---|
| 1 | agtcctgcgt ccgggccccg aggcgcagca gggcaccagg tggagcacca gctacgcgtg | |
| 61 | gcgcagcgca gcgtccctag caccgagcct cccgcagccg ccgagatgct gcgaacagag | 121 |
| | agctgccgcc ccaggtcgcc cgccggacag gtggccgcgg cgtccccgct cctgctgctg | 181 |
| | ctgctgctgc tcgcctggtg cgcgggcgcc tgccgaggtg ctccaatatt acctcaagga | 241 |
| | ttacagcctg aacaacagct acagttgtgg aatgagatag atgatacttg ttcgtctttt | 301 |
| | ctgtccattg attctcagcc tcaggcatcc aacgcactgg aggagctttg ctttatgatt | 361 |
| | atgggaatgc taccaaagcc tcaggaacaa gatgaaaaag ataatactaa aaggttctta | 421 |
| | tttcattatt cgaagacaca gaagttgggc aagtcaaatg ttgtgtcgtc agttgtgcat | 481 |
| | ccgttgctgc agctcgttcc tcacctgcat gagagaagaa tgaagagatt cagagtggac | 541 |
| | gaagaattcc aaagtccctt tgcaagtcaa agtcgaggat attttttatt caggccacgg | 601 |
| | aatggaagaa ggtcagcagg gttcatttaa aatggatgcc agctaatttt ccacagagca | 661 |
| | atgctatgga atacaaaatg tactgacatt ttgttttctt ctgaaaaaaa tccttgctaa | 721 |
| | atgtactctg ttgaaaatcc ctgtgttgtc aatgttctca gttgtaacaa tgttgtaaat | 781 |
| | gttcaatttg ttgaaaatta aaaaatctaa aaataaa | |

Homo sapiens guanylate binding protein 5 (GBP5), mRNA
NCBI Reference Sequence: NM_052942.2 (SEQ ID. NO. 14)

| | | |
|---|---|---|
| 1 | ctccaggctg tggaaccttt gttctttcac tctttgcaat aaatcttgct gctgctcact | |
| 61 | ctttgggtcc acactgcctt tatgagctgt aacactcact gggaatgtct gcagcttcac | 121 |
| | tcctgaagcc agcgagacca cgaacccacc aggaggaaca aacaactcca gacgcgcagc | 181 |
| | cttaagagct gtaacactca ccgcgaaggt ctgcagcttc actcctgagc cagccagacc | 241 |
| | acgaacccac cagaaggaag aaactccaaa cacatccgaa catcagaagg agcaaactcc | 301 |
| | tgacacgcca cctttaagaa ccgtgacact caacgctagg gtccgcggct tcattcttga | 361 |
| | agtcagtgag accaagaacc caccaattcc ggacacgcta attgttgtag atcatcactt | 421 |
| | caaggtgccc atatctttct agtggaaaaa ttattctggc ctccgctgca tacaaatcag | 481 |
| | gcaaccagaa ttctacatat ataaggcaaa gtaacatcct agacatggct ttagagatcc | 541 |
| | acatgtcaga ccccatgtgc ctcatcgaga actttaatga gcagctgaag gttaatcagg | 601 |
| | aagctttgga gatcctgtct gccattacgc aacctgtagt tgtggtagcg attgtgggcc | 661 |
| | tctatcgcac tggcaaatcc tacctgatga acaagctggc tgggaagaac aagggcttct | 721 |
| | ctgttgcatc tacggtgcag tctcacacca agggaatttg gatatggtgt gtgcctcatc | 781 |
| | ccaactggcc aaatcacaca ttagttctgc ttgacaccga gggcctggga gatgtagaga | 841 |
| | aggctgacaa caagaatgat atccagatct ttgcactggc actcttactg agcagcacct | 901 |
| | ttgtgtacaa tactgtgaac aaaattgatc agggtgctat cgacctactg cacaatgtga | 961 |
| | cagaactgac agatctgctc aaggcaagaa actcacccga ccttgacagg gttgaagatc | 1021 |
| | ctgctgactc tgcgagcttc ttcccagact tagtgtggac tctgagagat ttctgcttag | 1081 |
| | gcctggaaat agatgggcaa cttgtcacac cagatgaata cctggagaat tccctaaggc | 1141 |
| | caaagcaagg tagtgatcaa agagttcaaa atttcaattt gccccgtctg tgtatacaga | 1201 |
| | agttctttcc aaaaaagaaa tgctttatct ttgacttacc tgctcaccaa aaaaagcttg | 1261 |
| | cccaacttga aacactgcct gatgatgagc tagagcctga atttgtgcaa caagtgacag | 1321 |
| | aattctgttc ctacatcttt agccattcta tgaccaagac tcttccaggt ggcatcatgg | 1381 |
| | tcaatggatc tcgtctaaag aacctggtgc tgacctatgt caatgccatc agcagtgggg | 1441 |
| | atctgccttg catagagaat gcagtcctgg ccttggctca gagagagaac tcagctgcag | 1501 |
| | tgcaaaaggc cattgcccac tatgaccagc aaatgggcca gaaagtgcag ctgcccatgg | 1561 |
| | aaaccctcca ggagctgctg gacctgcaca ggaccagtga gagggaggcc attgaagtct | 1621 |
| | tcatgaaaaa ctctttcaag gatgtagacc aaagtttcca gaaagaattg gagactctac | 1681 |
| | tagatgcaaa acagaatgac atttgtaaac ggaacctgga agcatcctcg gattattgct | 1741 |
| | cggctttact taaggatatt tttggtcctc tagaagaagc agtgaagcag ggaatttatt | 1801 |
| | ctaagccagg aggccataat ctcttcattc agaaaacaga agaactgaag gcaaagtact | 1861 |
| | atcgggagcc tcggaaagga atacaggctg aagaagttct gcagaaatat ttaaagtcca | 1921 |
| | aggagtctgt gagtcatgca atattacaga ctgaccaggc tctcacagag acggaaaaaa | 1981 |
| | agaagaaaga ggcacaagtg aaagcagaag ctgaaaaggc tgaagcgcaa aggttggcgg | 2041 |
| | cgattcaaag gcagaacgag caaatgatgc aggagaggga gagactccat caggaacaag | 2101 |
| | tgagacaaat ggagatagcc aaacaaaatt ggctggcaga gcaacagaaa atgcaggaac | 2161 |
| | aacagatgca ggaacaggct gcacagctca gcacaacatt ccaagctcaa aatagaagcc | 2221 |
| | ttctcagtga gctccagcac gcccagagga ctgttaataa cgatgatcca tgtgttttac | 2281 |
| | tctaaagtgc taaatatggg agtttccttt ttttactctt tgtcactgat gacacaacag | 2341 |
| | aaaagaaact gtagaccttg ggacaatcaa catttaaata aactttataa ttattttttc | 2401 |
| | aaactttaaa aaaaaaaaaa aaaaaaaaaa a | |

Homo sapiens topoisomerase (DNA) II alpha 170 kDa (TOP2A), mRNA
NCBI Reference Sequence: NM_001067.2 (SEQ ID. NO. 15)

| | | |
|---|---|---|
| 1 | aggttcaagt ggagctctcc taaccgacgc gcgtctgtgg agaagcggct tggtcggggg | |
| 61 | tggtctcgtg gggtcctgcc tgtttagtcg ctttcagggt tcttgagccc cttcacgacc | 121 |
| | gtcaccatgg aagtgtcacc attgcagcct gtaaatgaaa atatgcaagt caacaaaata | 181 |
| | aagaaaaatg aagatgctaa gaaaagactg tctgttgaaa gaatctatca aaagaaaaca | 241 |
| | caattggaac atattttgct ccgcccagac acctacattg gttctgtgga attagtgacc | 301 |
| | cagcaaatgt gggtttacga tgaagatgtt ggcattaact atagggaagt cacttttgtt | 361 |
| | cctggtttgt acaaaatctt tgatgagatt ctagttaatg ctgcggacaa caaacaaagg | 421 |
| | gacccaaaaa tgtcttgtat tagagtcaca attgatccgg aaaacaattt aattagtata | 481 |
| | tggaataatg gaaaaggtat tcctgttgtt gaacacaaag ttgaaaagat gtatgtccca | 541 |
| | gctctcatat ttggacagct cctaacttct agtaactatg atgatgatga aaagaaagtg | 601 |
| | acaggtggtc gaaatggcta tggagccaaa ttgtgtaaca tattcagtac caaatttact | 661 |
| | gtggaaacag ccagtagaga atacaagaaa atgttcaaac agacatggat ggataatatg | 721 |
| | ggaagagctg gtgagatgga actcaagccc ttcaatggag aagattatac atgtatcacc | 781 |
| | tttcagcctg atttgtctaa gtttaaaatg caaagcctgg acaaagatat tgttgcacta | 841 |
| | atggtcagaa gagcatatga tattgctgga tccaccaaag atgtcaaagt ctttcttaat | 901 |
| | ggaaataaac tgccagtaaa aggatttcgt agttatgtgg acatgtattt gaaggacaag | 961 |
| | ttggatgaaa ctggtaactc cttgaaagta atacatgaac aagtaaacca caggtgggaa | 1021 |
| | gtgtgtttaa ctatgagtga aaaaggcttt cagcaaatta gctttgtcaa cagcattgct | 1081 |
| | acatccaagg gtggcagaca tgttgattat gtagctgatc agattgtgac taaacttgtt | 1141 |
| | gatgttgtga agaagaagaa caagggtggt gttgcagtaa aagcacatca ggtgaaaaat | 1201 |
| | cacatgtgga tttttgtaaa tgccttaatt gaaaacccaa cctttgactc tcagacaaaa | 1261 |
| | gaaaacatga ctttacaacc caagagcttt ggatcaacat gccaattgag tgaaaaattt | 1321 |
| | atcaaagctg ccattggctg tggtattgta gaaagcatac taaactgggt gaagtttaag | 1381 |
| | gcccaagtcc agttaaacaa gaagtgttca gctgtaaaac ataatagaat caagggaatt | 1441 |
| | cccaaactcg atgatgccaa tgatgcaggg ggccgaaact ccactgagtg tacgcttatc | 1501 |
| | ctgactgagg gagattcagc caaaactttg gctgtttcag gccttggtgt ggttgggaga | 1561 |
| | gacaaatatg gggttttccc tcttagagga aaaatactca atgttcgaga agcttctcat | 1621 |
| | aagcagatca tggaaaatgc tgagattaac aatatcatca agattgtggg tcttcagtac | 1681 |
| | aagaaaaact atgaagatga agattcattg aagacgcttc gttatgggaa gataatgatt | 1741 |
| | atgacagatc aggaccaaga tggttcccac atcaaaggct tgctgattaa ttttatccat | 1801 |
| | cacaactggc cctctcttct gcgacatcgt tttctggagg aatttatcac tcccattgta | 1861 |
| | aaggtatcta aaaacaagca agaaatggca ttttacagcc ttcctgaatt tgaagagtgg | 1921 |
| | aagagttcta ctccaaatca taaaaaatgg aaagtcaaat attacaaagg tttgggcacc | 1981 |
| | agcacatcaa aggaagctaa agaatacttt gcagatatga aaagacatcg tatccagttc | 2041 |
| | aaatattctg gtcctgaaga tgatgctgct atcagcctgg cctttagcaa aaaacagata | 2101 |
| | gatgatcgaa aggaatggtt aactaatttc atggaggata gaagacaacg aaagttactt | 2161 |
| | gggcttcctg aggattactt gtatggacaa actaccacat atctgacata taatgacttc | 2221 |
| | atcaacaagg aacttatctt gttctcaaat tctgataacg agagatctat cccttctatg | 2281 |
| | gtggatggtt tgaaaccagg tcagagaaag gttttgttta cttgcttcaa acggaatgac | 2341 |
| | aagcgagaag taaaggttgc ccaattagct ggatcagtgg ctgaaatgtc ttcttatcat | 2401 |
| | catggtgaga tgtcactaat gatgaccatt atcaatttgg ctcagaattt tgtgggtagc | 2461 |
| | aataatctaa acctcttgca gcccattggt cagtttggta ccaggctaca tggtggcaag | 2521 |
| | gattctgcta gtccacgata catctttaca atgctcagct ctttggctcg attgttattt | 2581 |
| | ccaaaaaaag atgatcacac gttgaagttt ttatatgatg acaaccagcg tgttgagcct | 2641 |
| | gaatggtaca ttcctattat tcccatggtg ctgataaatg gtgctgaagg aatcggtact | 2701 |
| | gggtggtcct gcaaaatccc caactttgat gtgcgtgaaa ttgtaaataa catcaggcgt | 2761 |
| | ttgatggatg gagaagaacc tttgccaatg cttccaagtt acaagaactt caagggtact | 2821 |
| | attgaagaac tggctccaaa tcaatatgtg attagtggtg aagtagctat tcttaattct | 2881 |
| | acaaccattg aaatctcaga gcttcccgtc agaacatgga cccagacata caaagaacaa | 2941 |
| | gttctagaac ccatgttgaa tggcaccgag aagacacctc ctctcataac agactatagg | 3001 |
| | gaataccata cagataccac tgtgaaattt gttgtgaaga tgactgaaga aaaactggca | 3061 |
| | gaggcagaga gagttggact acacaaagtc ttcaaactcc aaactagtct cacatgcaac | 3121 |
| | tctatggtgc tttttgacca cgtaggctgt ttaaagaaat atgacacggt gttggatatt | 3181 |
| | ctaagagact tttttgaact cagacttaaa tattatggat taagaaaaga atggctccta | 3241 |
| | ggaatgcttg gtgctgaatc tgctaaactg aataatcagg ctcgctttat cttagagaaa | 3301 |
| | atagatggca aaataatcat tgaaaataag cctaagaaag aattaattaa agttctgatt | 3361 |
| | cagaggggat atgattcgga tcctgtgaag gcctggaaag aagcccagca aaaggttcca | 3421 |
| | gatgaagaag aaaatgaaga gagtgacaac gaaaaggaaa ctgaaaagag tgactccgta | 3481 |
| | acagattctg gaccaacctt caactatctt cttgatatgc ccctttggta tttaaccaag | 3541 |
| | gaaaagaaag atgaactctg caggctaaga aatgaaaaag aacaagagct ggacacatta | 3601 |
| | aaaagaaaga gtccatcaga tttgtggaaa gaagacttgg ctacatttat tgaagaattg | 3661 |
| | gaggctgttg aagccaagga-aaaacaagat gaacaagtcg gacttcctgg gaaagggggg | 3721 |
| | aaggccaagg ggaaaaaaac acaaatggct gaagttttgc cttctccgcg tggtcaaaga | 3781 |
| | gtcattccac gaataaccat agaaatgaaa gcagaggcag aaaagaaaaa taaaaagaaa | 3841 |
| | attaagaatg aaaatactga aggaagccct caagaagatg gtgtggaact agaaggccta | 3901 |
| | aaacaaagat tagaaaagaa acagaaaaga gaaccaggta caaagacaaa gaaacaaact | 3961 |
| | acattggcat ttaagccaat caaaaaagga aagaagagaa atccctggtc tgattcagaa | 4021 |
| | tcagatagga gcagtgacga aagtaatttt gatgtccctc cacgagaaac agagccacgg | 4081 |
| | agagcagcaa caaaaacaaa attcacaatg gatttggatt cagatgaaga tttctcagat | 4141 |
| | tttgatgaaa aaactgatga tgaagatttt gtcccatcag atgctagtcc acctaagacc | 4201 |
| | aaaacttccc caaaacttag taacaaagaa ctgaaaccac agaaaagtgt cgtgtcagac | 4261 |
| | cttgaagctg atgatgttaa gggcagtgta ccactgtctt caagccctcc tgctacacat | 4321 |
| | ttcccagatg aaactgaaat tacaaaccca gttcctaaaa agaatgtgac agtgaagaag | 4381 |
| | acagcagcaa aaagtcagtc ttccacctcc actaccggtg ccaaaaaaag ggctgcccca | 4441 |
| | aaaggaacta aaagggatcc agctttgaat tctggtgtct ctcaaaagcc tgatcctgcc | 4501 |
| | aaaaccaaga atcgccgcaa aaggaagcca tccacttctg atgattctga ctctaatttt | 4561 |
| | gagaaaattg tttcgaaagc agtcacaagc aagaaatcca agggggagag tgatgacttc | 4621 |
| | catatggact ttgactcagc tgtggctcct cgggcaaaat ctgtacgggc aaagaaacct | 4681 |
| | ataaagtacc tggaagagtc agatgaagat gatctgtttt aaaatgtgag gcgattattt | 4741 |
| | taagtaatta tcttaccaag cccaagactg gttttaaagt tacctgaagc tcttaacttc | 4801 |
| | ctcccctctg aatttagttt ggggaaggtg tttttagtac aagacatcaa agtgaagtaa | 4861 |
| | agcccaagtg ttctttagct ttttataata ctgtctaaat agtgaccatc tcatgggcat | 4921 |
| | tgttttcttc tctgctttgt ctgtgttttg agtctgcttt cttttgtctt taaaacctga | 4981 |
| | tttttaagtt cttctgaact gtagaaatag ctatctgatc acttcagcgt aaagcagtgt | 5041 |
| | gtttattaac catccactaa gctaaaacta gagcagtttg atttaaaagt gtcactcttc | 5101 |
| | ctccttttct actttcagta gatatgagat agagcataat tatctgtttt atcttagttt | 5161 |
| | tatacataat ttaccatcag atagaacttt atggttctag tacagatact ctactacact | 5221 |
| | cagcctctta tgtgccaagt ttttctttaa gcaatgagaa attgctcatg ttcttcatct | 5281 |
| | tctcaaatca tcagaggcca aagaaaaaca ctttggctgt gtctataact tgacacagtc | 5341 |
| | aatagaatga agaaaattag agtagttatg tgattatttc agctcttgac ctgtcccctc | 5401 |
| | tggctgcctc tgagtctgaa tctcccaaag agagaaacca atttctaaga ggactggatt | 5461 |
| | gcagaagact cggggacaac atttgatcca agatcttaaa tgttatattg ataaccatgc | 5521 |
| | tcagcaatga gctattagat tcattttggg aaatctccat aatttcaatt tgtaaacttt | 5581 |
| | gttaagacct gtctacattg ttatatgtgt gtgacttgag taatgttatc aacgtttttg | 5641 |
| | taaatattta ctatgttttt ctattagcta aattccaaca attttgtact ttaataaa | |

Homo sapiens serpin peptidase inhibitor, clade B (ovalbumin), member 4
(SERPINB4), mRNA
NCBI Reference Sequence: NM_002974.2 (SEQ ID. NO. 16)

| | | |
|---|---|---|
| 1 | ccttcattcc acagacacac acagcctctc tgcccacctc tgcttcctct aggaacacag | |
| 61 | gagttccaga tcacatcgag ttcaccatga attcactcag tgaagccaac accaagttca | 121 |
| | tgttcgatct gttccaacag ttcagaaaat caaaagagaa caacatcttc tattccccta | 181 |
| | tcagcatcac atcagcatta gggatggtcc tcttaggagc caaagacaac actgcacaac | 241 |
| | aaattagcaa ggttcttcac tttgatcaag tcacagagaa caccacagaa aaagctgcaa | 301 |
| | catatcatgt tgataggtca ggaaatgttc atcaccagtt tcaaaagctt ctgactgaat | 361 |
| | tcaacaaatc cactgatgca tatgagctga agatcgccaa caagctcttc ggagaaaaga | 421 |
| | cgtatcaatt tttacaggaa tatttagatg ccatcaagaa attttaccag accagtgtgg | 481 |
| | aatctactga ttttgcaaat gctccagaag aaagtcgaaa gaagattaac tcctgggtgg | 541 |
| | aaagtcaaac gaatgaaaaa attaaaaacc tatttcctga tgggactatt ggcaatgata | 601 |
| | cgacactggt tcttgtgaac gcaatctatt tcaaagggca gtgggagaat aaatttaaaa | 661 |
| | aagaaaacac taaagaggaa aaattttggc caaacaagaa tacatacaaa tctgtacaga | 721 |
| | tgatgaggca atacaattcc tttaattttg ccttgctgga ggatgtacag gccaaggtcc | 781 |
| | tggaaatacc atacaaaggc aaagatctaa gcatgattgt gctgctgcca aatgaaatcg | 841 |
| | atggtctgca gaagcttgaa gagaaactca ctgctgagaa attgatggaa tggacaagtt | 901 |
| | tgcagaatat gagagagaca tgtgtcgatt tacacttacc tcggttcaaa atggaagaga | 961 |
| | gctatgacct caaggacacg ttgagaacca tgggaatggt gaatatcttc aatggggatg | 1021 |
| | cagacctctc aggcatgacc tggagccacg gtctctcagt atctaaagtc ctacacaagg | 1081 |
| | cctttgtgga ggtcactgag gagggagtgg aagctgcagc tgccaccgct gtagtagtag | 1141 |
| | tcgaattatc atctccttca actaatgaag agttctgttg taatcaccct ttcctattct | 1201 |
| | tcataaggca aaataagacc aacagcatcc tcttctatgg cagattctca tccccataga | 1261 |
| | tgcaattagt ctgtcactcc atttagaaaa tgttcaccta gaggtgttct ggtaaactga | 1321 |
| | ttgctggcaa caacagattc tcttggctca tatttctttt ctatctcatc ttgatgatga | 1381 |
| | tagtcatcat caagaattta atgattaaaa tagcatgcct ttctctcttt ctcttaataa | 1441 |
| | gcccacatat aaatgtactt ttccttccag aaaaatttcc cttgaggaaa aatgtccaag | 1501 |
| | ataagatgaa tcatttaata ccgtgtcttc taaatttgaa atataattct gtttctgacc | 1561 |
| | tgttttaaat gaaccaaacc aaatcatact ttctcttcaa atttagcaac ctagaaacac | 1621 |
| | acatttcttt gaatttaggt gatacctaaa tccttcttat gtttctaaat tttgtgattc | 1681 |
| | tataaaacac atcatcaata aaataatgac ataaaatcaa aaaaaaaaaa aaaaaa | |

Homo sapiens granulysin (GNLY), transcript variant 519, mRNA
NCBI Reference Sequence: NM_012483.1 (SEQ ID. NO. 17)

| | | |
|---|---|---|
| 1 | cctgggccct cctgctcctt gcagccatgc tcctgggcaa cccagcccct gcctccgcat | |
| 61 | ctgcgtggtg aaggccattg gcctcatcgg tggatctgcg tttcctcggg cccacactgt | 121 |
| | ctaggattgt gcggggctgg tgagagaaca agatctcttc cgtgttcaag gcagacttcc | 181 |
| | tgccccctgc accctgctct ctcccgggcc ttgaggtcag tgtgagcccc aagggcaaga | 241 |
| | acacttctgg aagggagagt ggatttggct gggcctctgg atggaaggtc tggtcttctc | 301 |
| | tcgtctgagc cctgagtact acgacccggc aagagcccac ctgcgtgatg gggagaaatc | 361 |
| | ctgcccgtgc gggcaggagg gcccccaggg tgacctgttg accaaaacac aggagctggg | 421 |
| | ccgtgactac aggacctgtc tgacgatagt ccaaaaactg aagaagatgg tggataagcc | 481 |
| | cacccagaga agtgtttcca atgctgcgac ccgggtgtgt aggacgggga ggtcacgatg | 541 |
| | gcgcgacgtc tgcagaaatt tcatgaggag gtatcagtct agagttatcc aaggcctcgt | 601 |
| | ggccggagaa actgcccagc agatctgtga ggacctcagg ttgtgtatac cttctacagg | 661 |
| | tcccctctga gccctctcac cttgtcctgt ggaagaagca caggctcctg tcctcagatc | 721 |
| | ccgggaacgt cagcaacctc tgccggctcc tcgcttcctc gatccagaat ccactctcca | 781 |
| | gtctccctcc cctgactccc tctgctgtcc tcccctctca ggggaataaa gtgtcaagca | 841 |
| | agattttagc cgc | |

Homo sapiens gametocyte specific factor 1 (GTSF1), mRNA
NCBI Reference Sequence: NM_144594.1 (SEQ ID. NO. 18)

| | | |
|---|---|---|
| 1 | agcggagggg tgtgtccacc gagcacttgg attcagcttc ttcatttcca acatggaaga | |
| 61 | aacttacacc gactccctgg accctgagaa gctattgcaa tgcccctatg acaaaaacca | 121 |
| | tcaaatcagg gcttgcaggt ttccttatca tcttatcaag tgcagaaaga atcatcctga | 181 |
| | tgttgcaagc aaattggcta cttgtccctt caatgctcgc caccaggttc ctcgagctga | 241 |
| | aattagtcat catatctcaa gctgtgatga cagaagttgt attgagcaag atgttgtcaa | 301 |
| | ccaaaccagg agccttagac aagagactct ggctgagagc acttggcagt gccctccttg | 361 |
| | cgatgaagac tgggataaag atttgtggga gcagaccagc accccatttg cctggggcac | 421 |
| | aactcactac tctgacaaca acagccctgc gagcaacata gttacagaac ataagaataa | 481 |
| | cctggcttca ggcatgcgag ttcccaaatc tctgccgtat gttctgccat ggaaaaacaa | 541 |
| | tggaaatgca cagtaactga atacctatct catcaaatgc cagaccctag aagactgttg | 601 |
| | cttcttcttc taccagtggg ttctcatttt cctcctaatc taattataga atagtaaact | 661 |
| | ccctgtgact ttccaaactg acaagcacac ttttttcctc cccccttgaa tcctcattta | 721 |
| | atgcaagaac cctcatactc agaagcttcc aaataaacct ttgatacaga aaaaaaaaaa | 781 |
| | aaaaa | |

Homo sapiens peptidase inhibitor 3, skin-derived (SKALP) (PI3), mRNA
NCBI Reference Sequence: NM_002638.2 (SEQ ID. NO. 19)

| | | |
|---|---|---|
| 1 | aggccaagct ggactgcata aagattggta tggccttagc tcttagccaa acaccttcct | |
| 61 | gacaccatga gggccagcag cttcttgatc gtggtggtgt tcctcatcgc tgggacgctg | 121 |
| | gttctagagg cagctgtcac gggagttcct gttaaaggtc aagacactgt caaaggccgt | 181 |
| | gttccattca atggacaaga tcccgttaaa ggacaagttt cagttaaagg tcaagataaa | 241 |
| | gtcaaagcgc aagagccagt caaaggtcca gtctccacta agcctggctc ctgccccatt | 301 |
| | atcttgatcc ggtgcgccat gttgaatccc cctaaccgct gcttgaaaga tactgactgc | 361 |
| | ccaggaatca agaagtgctg tgaaggctct tgcgggatgg cctgtttcgt tccccagtga | 421 |
| | gagggagccg gtccttgctg cacctgtgcc gtccccagag ctacaggccc catctggtcc | 481 |
| | taagtccctg ctgcccttcc ccttcccaca ctgtccattc ttcctcccat tcaggatgcc | 541 |
| | cacggctgga gctgcctctc tcatccactt tccaataaag agttccttct gctccaaaaa | 601 |
| | aaaaaaaaaa aaaaaaaaaa aaa | |

Homo sapiens S100 calcium binding protein A7A (S100A7A), mRNA
NCBI Reference Sequence: NM_176823.3 (SEQ ID. NO. 20)

| | | |
|---|---|---|
| 1 | atctcactca tccttctact cgtgacactt cccagttctg gctttttgaa agcaaagatg | |
| 61 | agcaacactc aagctgagag gtccataata ggcatgatcg acatgtttca caaatacacc | 121 |
| | ggacgtgatg gcaagattga gaagccaagc ctgctgacga tgatgaagga gaacttcccc | 181 |
| | aatttcctca gtgcctgtga caaaaagggc atacattacc tcgccactgt ctttgagaaa | 241 |
| | aaggacaaga atgaggataa gaagattgat ttttctgagt ttctgtcctt gctgggagac | 301 |
| | atagccgcag actaccacaa gcagagccat ggagcggcgc cctgttctgg gggaagccag | 361 |
| | tgatccagcc ccaccaaggg gcctccagag accccaggaa caataagtgt ctcctcccac | 421 |
| | cagacacttg ccttatttct tcttctcttt ggtgacctac attgtcaaaa ctaccaattc | 481 |
| | caggttaact ttgttggaga atttccccca cccccatcca gtgggtcacc caggagtaat | 541 |
| | gtccctccag caacgttccc cctatggcct ccagcagagc tgatctgcct ctcacacagg | 601 |
| | tcctggtgtc tgcctctgca ccgttcccta aatgcagcca ccttggcagg ttccaggtgg | 661 |
| | aagttggtag aaggcccctg ccaggtcaca gcaatgctct ccttgtcaag gcatggacca | 721 |
| | gggtcattca gacacattca gatactgcac tgagaaggag ctggcatctc tcagtgtgct | 781 |
| | cctgccctcc cactcctgcc ccagctgttc tccagggctt ggggaaacag aaaccactca | 841 |
| | catagggatt cctggatggc ttcaggttca gcgcccttgg ggctatgaat gggaggctca | 901 |
| | gcagtgccct gaggatgggc ttccttgtcc tgtggcctct gctccagggg cagtgtcctt | 961 |
| | tccctgtgct gtgtgcttgt gtgcatgtgc ctatgtgggt gaccctgtgg aagtgagaag | 1021 |
| | gagtcactgt gatgcttagc tgtcctaaat gatggtttgc tcaatgccag gactgggttt | 1081 |
| | ctggtgatga atgaatattc cagattttga ggagctctaa gtggtccagg agtccaagta | 1141 |
| | agcagtctgg ctggaataag gcagcatcac ggaaattcg taaggactga cacagagagc | 1201 |
| | tcatgctgac tgtgatgaga aattgcagca cctctatatc gcaggtaatg gagtagtttg | 1261 |
| | ttattggtag tctactccag gccaggcagt gtgttatggg ctgaggatgc agaaacaggc | 1321 |
| | aggacacagt gctgtcctag cagtgcactg gcgggtctct ccatgcaggc cacaacacag | 1381 |
| | ggtcagtgtt cacctggtgt cacttccagg caatgttctg tgcagccgct cttagtattc | 1441 |
| | ttccttgagg ctcacatcat gtgtccctat cactcttact actctggtca gtctccagct | 1501 |
| | aacctctcaa tcaggcaaac attcttcttg gaggaatcag gcaaacatct caaaaattct | 1561 |
| | ctttccatcc taccagcagc agtgtgtaag atgggctatt tgttctttgg aatgactgct | 1621 |
| | ccactccaca ctcacacctc tattcacaga ccagcatctc ctctccttat caggaacatt | 1681 |
| | ccttcctgaa catattctgc acctcgtcag ccttcaggac tgatctgcca ttttcacctc | 1741 |
| | taaatcccca tgtctgacca ttagttttct tctatttcct tctctccctt tctcattctc | 1801 |
| | attccacctg ttcttggaac tcacggagac ctacagtccc tgggctttca ttttctcctc | 1861 |
| | ccagccccct gctgccttct ctatgcagcc tgccctccat catccacccc agaattgctc | 1921 |
| | tctttcctct cttagctctg ttgcccactt tccttgggcc ataccttccc tgcagatctc | 1981 |
| | cagcccagaa ccatcttccc ctgttgtcct cctctctcct ccaccgggac tgctggtcac | 2041 |
| | tgcttagaac cgtcatgcca gggtcccaaa agtgtgggtg cctgacttcc tctctgtgca | 2101 |
| | gcactctctg aatccctcct attcaccttg ctgctgttat tccccgaatg cgcaacatac | 2161 |
| | cccccatcaa tatatctcag tatttcatgt ctcaatacca atcttttaaa ctactgcctc | 2221 |
| | taccagaaat gtcttttaat acttcttctg tctcattaac attacatttc aaggctgagc | 2281 |
| | tttaatgtca gtgtctctta gacattcaga gggtgaacca ccatcccttc accccaaaga | 2341 |
| | aatgatctct gcttcatttg tgcctccctc accatgaccc cactcttacc atagtggcta | 2401 |
| | cattacttca gattccccta atgtctttcc agccagactt ggaatcatgg agggaaaaca | 2461 |
| | ttgttacctc ggatctcctg gttacccagc acatagtagt actggattcc agctcataat | 2521 |
| | aagtactcta tatcattttt caatataaaa tgtatttgtg caaattctag tcaatactac | 2581 |
| | tttatgtaaa cagcagtgta aaatccaaaa acttccagtc ctggaggcag gttgtgcagc | 2641 |
| | ttaggggagg accccagaat ctggacccca gagtctggaa gcaggccaga aaggataagg | 2701 |
| | caaatgactg aacagttccc tcaggactca cgtactgatc tcccaaaaga agagagggtc | 2761 |
| | tccctggggt ggggttgctg gaccttcaat ccatcgctac agtccagaag gcaattggcc | 2821 |
| | actcctaatg tgggcctgcc ctccctttat ttttccagtt cttatttcac ctgataatat | 2881 |
| | tccgtccaat tggcaatggc acataaaaat taggatggag tgtgtggaca aatacttctt | 2941 |
| | catcttcttg tctaggtttt agaaatcacc ttctcaaggg agccttgtct aatgttcctg | 3001 |
| | agactatttc acactctcca tgcttatgtc aatgcaggac tcatcacatc tattcggata | 3061 |
| | ttctgtttac acacccatgt catcccagag aggtgatcac agggcaggga cacatgtgtg | 3121 |
| | gcatacagtt cctagttaag atcccaaatc ctgagatatt gctgatttgc tatggcaggt | 3181 |
| | cgtcaagaga actgtgtcat tccaaactca ccaaggtggc ttatagaaca gaagcagatg | 3241 |
| | gatatgaaga ggagagggga ccagaccatc tccgcaacca cagcccagag ctccagtcac | 3301 |
| | cagatagaaa attgatttga tttcatccaa tattccttcg aaagagtgtc aaggaatagg | 3361 |
| | gtggggcaat gtgtcattct gcattggaag gaggacattt tagagcaagg cctaagggca | 3421 |
| | caggtattag tgtcatattg atcagaattc aacctttgtt ctaacacata ctagagcaag | 3481 |
| | aatttacttg atttggaata attaatagct actggacatt atattggtac taaagagaaa | 3541 |
| | gaatacttga cagctctatg cccacactca cattacagct gatgtgaaag agattctgga | 3601 |
| | aatccaaatg ttccccagaa attctgatat caaaacattc caataacttt tttttttcag | 3661 |
| | gcgcagtctc actctgtcgc ccgggctgga gtgctgtgag ctgtccgtgg tgctgaattc | 3721 |
| | actgtgacgt cactcctgtc tctctttgct ttcttctgac tgacatttat tcagccttct | 3781 |
| | ctacaggaat ctcttatgtt cccccacatg caggtggttt ttcagtaggc tcctgaagag | 3841 |
| | tgatctcaac tttccaggaa gaaaagaggg caaagggaac aatgtgaaaa gaagcagaaa | 3901 |
| | atcataaaag accatgtgtt tgataaacaa ccagattgtt tctggttccc tgccactata | 3961 |
| | aaaacaccat gagagcatac tcatacatgt tcccttataa atctgcgagg tagtttcttt | 4021 |
| | ggtattcttg cccaggaaat gggttgattc atcacagatt ttatatatat actttttttt | 4081 |
| | aactaagtgt gagataatat cttattgttt ttgtaacttg cattttacaa gagttctgac | 4141 |
| | cagcaccaga taagcttcag tgctctcctt tctttggcct taatattatt ggattaaaga | 4201 |
| | attactgcct ctcactagga gcatcattta tttaccatta ttttcaattt catattaaaa | 4261 |
| | ctcaatttct agtagagtc | |

Homo sapiens indoleamine 2,3-dioxygenase 1 (IDO1), mRNA
NCBI Reference Sequence: NM_002164.4 (SEQ ID. NO. 21)

| | | |
|---|---|---|
| 1 | aatttctcac tgcccctgtg ataaactgtg gtcactggct gtggcagcaa ctattataag | |
| 61 | atgctctgaa aactcttcag acactgaggg gcaccagagg agcagactac aagaatggca | 121 |
| | cacgctatgg aaaactcctg gacaatcagt aaagagtacc atattgatga agaagtgggc | 181 |
| | tttgctctgc caaatccaca ggaaaatcta cctgattttt ataatgactg gatgttcatt | 241 |
| | gctaaacatc tccgtgagtt catagagtct ggccagcttc gagaaagagt tgagaagtta | 301 |
| | aacatgctca gcattgatca tctcacagac cacaagtcac agcgccttgc acgtctagtt | 361 |
| | ctgggatgca tcaccatggc atatgtgtgg ggcaaaggtc atggagatgt ccgtaaggtc | 421 |
| | ttgccaagaa atattgctgt tccttactgc caactctcca agaaactgga actgcctcct | 481 |
| | attttggttt atgcagactg tgtcttggca aactggaaga aaaaggatcc taataagccc | 541 |
| | ctgacttatg agaacatgga cgttttgttc tcatttcgtg atggagactg cagtaaagga | 601 |
| | ttcttcctgg tctctctatt ggtggaaata gcagctgctt ctgcaatcaa agtaattcct | 661 |
| | actgtattca aggcaatgca aatgcaagaa cgggacactt tgctaaaggc gctgttggaa | 721 |
| | atagcttctt gcttggagaa agcccttcaa gtgtttcacc aaatccacga tcatgtgaac | 781 |
| | ccaaaagcat ttttcagtgt tcttcgcata tatttgtctg gctggaaagg caacccccag | 841 |
| | ctatcagacg gtctggtgta tgaagggttc tgggaagacc caaaggagtt tgcagggggc | 901 |
| | agtgcaggcc aaagcagcgt ctttcagtgc tttgacgtcc tgctgggcat ccagcagact | 961 |
| | gctggtggag gacatgctgc tcagttcctc caggacatga gaagatatat gccaccagct | 1021 |
| | cacaggaact tcctgtgctc attagagtca aatccctcag tccgtgagtt tgtcctttca | 1081 |
| | aaaggtgatg ctggcctgcg ggaagcttat gacgcctgtg tgaaagctct ggtctccctg | 1141 |
| | aggagctacc atctgcaaat cgtgactaag tacatcctga ttcctgcaag ccagcagcca | 1201 |
| | aaggagaata agacctctga agacccttca aaactggaag ccaaaggaac tggaggcact | 1261 |
| | gatttaatga atttcctgaa gactgtaaga agtacaactg agaaatccct tttgaaggaa | 1321 |
| | ggttaatgta acccaacaag agcacatttt atcatagcag agacatctgt atgcattcct | 1381 |
| | gtcattaccc attgtaacag agccacaaac taatactatg caatgtttta ccaataatgc | 1441 |
| | aatacaaaag acctcaaaat acctgtgcat ttcttgtagg aaaacaacaa aaggtaatta | 1501 |
| | tgtgtaatta tactagaagt tttgtaatct gtatcttatc attggaataa aatgacattc | 1561 |
| | aataaataaa aa | |

Homo sapiens gap junction protein, beta 6 (GJB6), mRNA
NCBI Reference Sequence: NM_006783.2 (SEQ ID. NO. 22)

| | | |
|---|---|---|
| 1 | ctgggaagac gctggtcagt tcacctgccc cactggttgt tttttaaaca aattctgata | |
| 61 | caggcgacat cctcactgac cgagcaaaga ttgacattcg tatcatcact gtgcaccatt | 121 |
| | ggcttctagg cactccagtg gggtaggaga aggaggtctg aaaccctcgc agagggatct | 181 |
| | tgccctcatt ctttgggtct gaaacactgg cagtcgttgg aaacaggact cagggataaa | 241 |
| | ccagcgcaat ggattggggg acgctgcaca ctttcatcgg gggtgtcaac aaacactcca | 301 |
| | ccagcatcgg gaaggtgtgg atcacagtca tctttatttt ccgagtcatg atcctcgtgg | 361 |
| | tggctgccca ggaagtgtgg ggtgacgagc aagaggactt cgtctgcaac acactgcaac | 421 |
| | cgggatgcaa aaatgtgtgc tatgaccact ttttcccggt gtcccacatc cggctgtggg | 481 |
| | ccctccagct gatcttcgtc tccaccccag cgctgctggt ggccatgcat gtggcctact | 541 |
| | acaggcacga aaccactcgc aagttcaggc gaggagagaa gaggaatgat ttcaaagaca | 601 |
| | tagaggacat taaaaagcag aaggttcgga tagaggggtc gctgtggtgg acgtacacca | 661 |
| | gcagcatctt tttccgaatc atctttgaag cagcctttat gtatgtgttt tacttccttt | 721 |
| | acaatgggta ccacctgccc tgggtgttga aatgtgggat tgacccctgc cccaaccttg | 781 |
| | ttgactgctt tatttctagg ccaacagaga agaccgtgtt taccattttt atgatttctg | 841 |
| | cgtctgtgat ttgcatgctg cttaacgtgg cagagttgtg ctacctgctg ctgaaagtgt | 901 |
| | gttttaggag atcaaagaga gcacagacgc aaaaaaatca ccccaatcat gccctaaagg | 961 |
| | agagtaagca gaatgaaatg aatgagctga tttcagatag tggtcaaaat gcaatcacag | 1021 |
| | gtttcccaag ctaaacattt caaggtaaaa tgtagctgcg tcataaggag acttctgtct | 1081 |
| | tctccagaag gcaataccaa cctgaaagtt ccttctgtag cctgaagagt ttgtaaatga | 1141 |
| | ctttcataat aaatagacac ttgagttaac tttttgtagg atacttgctc cattcataca | 1201 |
| | caacgtaatc aaatatgtgg tccatctctg aaaacaagag actgcttgac aaaggagcat | 1261 |
| | tgcagtcact ttgacaggtt ccttttaagt ggactctctg acaaagtggg tactttctga | 1321 |
| | aaatttatat aactgttgtt gataaggaac atttatccag gaattgatac ttttattagg | 1381 |
| | aaaagatatt tttataggct tggatgtttt tagttctgac tttgaattta tataaagtat | 1441 |
| | ttttataatg actggtcttc cttacctgga aaaacatgcg atgttagttt tagaattaca | 1501 |
| | ccacaagtat ctaaatttgg aacttacaaa gggtctatct tgtaaatatt gttttgcatt | 1561 |
| | gtctgttggc aaatttgtga actgtcatga tacgcttaag gtggaaagtg ttcattgcac | 1621 |
| | aatatatttt tactgctttc tgaatgtaga cggaacagtg tggaagcaga aggctttttt | 1681 |
| | aactcatccg tttgccaatc attgcaaaca actgaaatgt ggatgtgatt gcctcaataa | 1741 |
| | agctcgtccc cattgcttaa gccttcaaaa aaaaaaaaaa aaaaaaaaaa aaaaaaaaaa | 1801 |
| | aaaaa | |

Homo sapiens calmodulin-like 3 (CALML3), mRNA
NCBI Reference Sequence: NM_005185.2 (SEQ ID. NO. 23)

| | | |
|---|---|---|
| 1 | gagacagccc gccggccgcc cggatctcca cctgccaccc cagagctggg acagcagccg | |
| 61 | ggctgcggca ctgggaggga gaccccacag tggcctcttc tgccacccac gcccccaccc | 121 |
| | ctggcatggc cgaccagctg actgaggagc aggtcacaga attcaaggag gccttctccc | 181 |
| | tgtttgacaa ggatggggac ggctgcatca ccacccgcga gctgggcacg gtcatgcggt | 241 |
| | ccctgggcca gaaccccacg gaggccgagc tgcgggacat gatgagtgag atcgaccggg | 301 |
| | acggcaacgg caccgtggac ttccccgagt tcctgggcat gatggccagg aagatgaagg | 361 |
| | acacggacaa cgaggaggag atccgcgagg ccttccgcgt gttcgacaag gacggcaacg | 421 |
| | gcttcgtcag cgccgccgag ctgcgacacg tcatgacccg gctgggggag aagctgagtg | 481 |
| | acgaggagtt ggacgagatg atccgggccg cggacacgga cggagacgga caggtgaact | 541 |
| | acgaggagtt tgtccgtgtg ctggtgtcca agtgaggccg gcgcccacca tgctcctggg | 601 |
| | cgcccacgcg gcccacaggg caagaacccg gggcctcccg cctcctcccc catccccctg | 661 |
| | cctcccctgg gcactgtggc ttcctcctgc gcctggttga ttcagcccac ctctctgcat | 721 |
| | cccgcttccc gcgtctcttc tctgcactcc tgccgacctt cccacctgct catctgaatg | 781 |
| | acacggaacg ctcccactgc aggcaaaccg tgacgccctc cccactcggg agaagcagag | 841 |
| | ctgaccttag gaccgagcac cagggcaggt tgcgctgact ctgcggccct ccaggacgga | 901 |
| | caccgggtga ccccttaggg ctctcaggca agatccctaa gaggcaccca atgcccaggc | 961 |
| | caggggggct gcagccctca gcccccgcca ggattcccgc aggctcctgg actggaagct | 1021 |
| | ccctccgcgg tcggattctg gagggtggga ggcatcttgg cctgcagtaa gcggtgctga | 1081 |
| | cggggactct ggccacagag gtcaggcctc ctgaaaacag cactgccttc cgcgctgccc | 1141 |
| | cagcttgccc cattccttgt ccgccaaccc accgtgattc atcttctgaa gctgggagtg | 1201 |
| | aaactgggtc agctgtaacc tgttcctatt catctggaag gagggaggct tggatgagca | 1261 |
| | ggggatgaga gctgcaggga aataaatgag atattcgtcc tt | |

Homo sapiens serpin peptidase inhibitor, clade B (ovalbumin), member 3
(SERPINB3), mRNA
NCBI Reference Sequence: NM_006919.1 (SEQ ID. NO. 24)

| | | |
|---|---|---|
| 1 | ctctctgccc acctctgctt cctctaggaa cacaggagtt ccagatcaca tcgagttcac | |
| 61 | catgaattca ctcagtgaag ccaacaccaa gttcatgttc gacctgttcc aacagttcag | 121 |
| | aaaatcaaaa gagaacacca tcttctattc ccctatcagc atcacatcag cattagggat | 181 |
| | ggtcctctta ggagccaaag acaacactgc acaacagatt aagaaggttc ttcactttga | 241 |
| | tcaagtcaca gagaacacca caggaaaagc tgcaacatat catgttgata ggtcaggaaa | 301 |
| | tgttcatcac cagtttcaaa agcttctgac tgaattcaac aaatccactg atgcatatga | 361 |
| | gctgaagatc gccaacaagc tcttcggaga aaaaacgtat ctatttttac aggaatattt | 421 |
| | agatgccatc aagaaatttt accagaccag tgtggaatct gttgattttg caaatgctcc | 481 |
| | agaagaaagt cgaaagaaga ttaactcctg ggtggaaagt caaacgaatg aaaaaattaa | 541 |
| | aaacctaatt cctgaaggta atattggcag caataccaca ttggttcttg tgaacgcaat | 601 |
| | ctatttcaaa gggcagtggg agaagaaatt taataaagaa gatactaaag aggaaaaatt | 661 |
| | ttggccaaac aagaatacat acaagtccat acagatgatg aggcaataca catcttttca | 721 |
| | ttttgcctcg ctggaggatg tacaggccaa ggtcctggaa ataccataca aaggcaaaga | 781 |
| | tctaagcatg attgtgttgc tgccaaatga aatcgatggt ctccagaagc ttgaagagaa | 841 |
| | actcactgct gagaaattga tggaatggac aagtttgcag aatatgagag agacacgtgt | 901 |
| | cgatttacac ttacctcggt tcaaagtgga agagagctat gacctcaagg acacgttgag | 961 |
| | aaccatggga atggtggata tcttcaatgg ggatgcagac ctctcaggca tgaccgggag | 1021 |
| | ccgcggtctc gtgctatctg gagtcctaca caaggccttt gtggaggtta cagaggaggg | 1081 |
| | agcagaagct gcagctgcca ccgctgtagt aggattcgga tcatcaccta cttcaactaa | 1141 |
| | tgaagagttc cattgtaatc accctttcct attcttcata aggcaaaata agaccaacag | 1201 |
| | catcctcttc tatggcagat tctcatcccc gtagatgcaa ttagtctgtc actccatttg | 1261 |
| | gaaaatgttc acctgcagat gttctggtaa actgattgct ggcaacaaca gattctcttg | 1321 |
| | gctcatattt cttttctttc tcatcttgat gatgatcgtc atcatcaaga atttaatgat | 1381 |
| | taaaatagca tgcctttctc tctttctctt aataagccca catataaatg tactttttct | 1441 |
| | tccagaaaaa ttctccttga ggaaaaatgt ccaaaataag atgaatcact taataccgta | 1501 |
| | tcttctaaat ttgaaatata attctgtttg tgacctgttt taaatgaacc aaaccaaatc | 1561 |
| | atactttttc tttgaattta gcaacctaga aacacacatt tctttgaatt taggtgatac | 1621 |
| | ctaaatcctt cttatgtttc taaattttgt gattctataa aacacatcat caataaaata | 1681 |
| | gtgacataaa atca | |

Homo sapiens chemokine (C—X—C motif) ligand 6 (granulocyte chemotactic
protein 2) (CXCL6), mRNA
NCBI Reference Sequence: NM_002993.2 (SEQ ID. NO. 25)

| | | |
|---|---|---|
| 1 | ccagtctccg cgcctccacc cagctcagga acccgcgaac cctctcttga ccactatgag | |
| 61 | cctcccgtcc agccgcgcgg cccgtgtccc gggtccttcg ggctccttgt gcgcgctgct | 121 |
| | cgcgctgctg ctcctgctga cgccgccggg gcccctcgcc agcgctggtc ctgtctctgc | 181 |
| | tgtgctgaca gagctgcgtt gcacttgttt acgcgttacg ctgagagtaa accccaaaac | 241 |
| | gattggtaaa ctgcaggtgt tccccgcagg cccgcagtgc tccaaggtgg aagtggtagc | 301 |
| | ctccctgaag aacgggaagc aagtttgtct ggacccggaa gccccttttc taaagaaagt | 361 |
| | catccagaaa attttggaca gtggaaacaa gaaaaactga gtaacaaaaa agaccatgca | 421 |
| | tcataaaatt gcccagtctt cagcggagca gttttctgga gatccctgga cccagtaaga | 481 |
| | ataagaagga agggttggtt tttttccatt ttctacatgg attccctact ttgaagagtg | 541 |
| | tgggggaaag cctacgcttc tccctgaagt ttacagctca gctaatgaag tactaatata | 601 |
| | gtatttccac tatttactgt tattttacct gataagttat tgaacccttt ggcaattgac | 661 |
| | catattgtga gcaaagaatc actggttatt agtctttcaa tgaatattga attgaagata | 721 |
| | actattgtat ttctatcata cattccttaa agtcttaccg aaaaggctgt ggatttcgta | 781 |
| | tggaaataat gttttattag tgtgctgttg agggaggtat cctgttgttc ttactcactc | 841 |
| | ttctcataaa ataggaaata ttttagttct gttttcttgg ggaatatgtt actctttacc | 901 |
| | ctaggatgct atttaagttg tactgtatta gaacactggg tgtgtcatac cgttatctgt | 961 |
| | gcagaatata tttccttatt cagaatttct aaaaatttaa gttctgtaag ggctaatata | 1021 |
| | ttctcttcct atggttttag atgtttgatg tcttcttagt atggcataat gtcatgattt | 1081 |
| | actcattaaa ctttgatttt gtatgctatt ttttcactat aggatgacta taattctggt | 1141 |
| | cactaaatat acactttaga tagatgaaga agcccaaaaa cagataaatt cctgattgct | 1201 |
| | aatttacata gaaatgtatt ctcttggttt tttaaataaa agcaaaatta acaatgatct | 1261 |
| | gtgctctgca aagttttgaa aatatatttg aacaatttga atataaattc atcatttagt | 1321 |
| | cctcaaaata tatacagcat tgctaagatt ttcagatatc tattgtggat cttttaaagg | 1381 |
| | ttttgaccat tttgttatga ggaattatac atgtatcaca ttcactatat taaaattgca | 1441 |
| | cttttatttt ttcctgtgtg tcatgttggt ttttggtact tgtattgtca tttggagaaa | 1501 |
| | caataaaaga tttctaaacc aaaaaaaaaa aaaaaaaaa | |

Homo sapiens olfactomedin 4 (OLFM4), mRNA
NCBI Reference Sequence: NM_006418.3 (SEQ ID. NO. 26)

| | | |
|---|---|---|
| 1 | atgaggcccg gcctctcatt tctcctagcc cttctgttct tccttggcca agctgcaggg | |
| 61 | gatttggggg atgtgggacc tccaattccc agccccggct tcagctcttt cccaggtgtt | 121 |
| | gactccagct ccagcttcag ctccagctcc aggtcgggct ccagcttcag ccgcagctta | 181 |
| | ggcagcggag gttctgtgtc ccagttgttt tccaatttca ccggctccgt ggatgaccgt | 241 |
| | gggacctgcc agtgctctgt ttccctgcca gacaccacct ttcccgtgga cagagtggaa | 301 |
| | cgcttggaat tcacagctca tgttctttct cagaagtttg agaaagaact ttccaaagtg | 361 |
| | agggaatatg tccaattaat tagtgtgtat gaaaagaaac tgttaaacct aactgtccga | 421 |
| | attgacatca tggagaagga taccatttct tacactgaac tggacttcga gctgatcaag | 481 |
| | gtagaagtga aggagatgga aaaactggtc atacagctga aggagagttt tggtggaagc | 541 |
| | tcagaaattg ttgaccagct ggaggtggag ataagaaata tgactctctt ggtagagaag | 601 |
| | cttgagacac tagacaaaaa caatgtcctt gccattcgcc gagaaatcgt ggctctgaag | 661 |
| | accaagctga aagagtgtga ggcctctaaa gatcaaaaca cccctgtcgt ccaccctcct | 721 |
| | cccactccag ggagctgtgg tcatggtggt gtggtgaaca tcagcaaacc gtctgtggtt | 781 |
| | cagctcaact ggagagggtt ttcttatcta tatggtgctt ggggtaggga ttactctccc | 841 |
| | cagcatccaa acaaaggact gtattgggtg gcgccattga atacagatgg gagactgttg | 901 |
| | gagtattata gactgtacaa cacactggat gatttgctat tgtatataaa tgctcgagag | 961 |
| | ttgaggatca cctatggcca aggtagtggt acagcagttt acaacaacaa catgtacgtc | 1021 |
| | aacatgtaca acaccgggaa tattgccaga gttaacctga ccaccaacac gattgctgtg | 1081 |
| | actcaaactc tccctaatgc tgcctataat aaccgctttt catatgctaa tgttgcttgg | 1141 |
| | caagatattg actttgctgt ggatgagaat ggattgtggg ttatttattc aactgaagcc | 1201 |
| | agcactggta acatggtgat tagtaaactc aatgacacca cacttcaggt gctaaacact | 1261 |
| | tggtatacca agcagtataa accatctgct tctaacgcct tcatggtatg tggggttctg | 1321 |
| | tatgccaccc gtactatgaa caccagaaca gaagagattt tttactatta tgacacaaac | 1381 |
| | acagggaaag agggcaaact agacattgta atgcataaga tgcaggaaaa agtgcagagc | 1441 |
| | attaactata acccttttga ccagaaactt tatgtctata acgatggtta ccttctgaat | 1501 |
| | tatgatcttt ctgtcttgca gaagccccag taagctgttt aggagttagg gtgaaagaga | 1561 |
| | aaatgtttgt tgaaaaaata gtcttctcca cttacttaga tatctgcagg ggtgtctaaa | 1621 |
| | agtgtgttca ttttgcagca atgtttaggt gcatagttct accacactag agatctagga | 1681 |
| | catttgtctt gatttggtga gttctcttgg gaatcatctg cctcttcagg cgcattttgc | 1741 |
| | aataaagtct gtctagggtg ggattgtcag aggtctaggg gcactgtggg cctagtgaag | 1801 |
| | cctactgtga ggaggcttca ctagaagcct taaattagga attaaggaac ttaaaactca | 1861 |
| | gtatggcgtc tagggattct ttgtacagga aatattgccc aatgactagt cctcatccat | 1921 |
| | gtagcaccac taattcttcc atgcctggaa gaaacctggg gacttagtta ggtagattaa | 1981 |
| | tatctggagc tcctcgaggg accaaatctc caactttttt ttcccctcac tagcacctgg | 2041 |
| | aatgatgctt tgtatgtggc agataagtaa atttggcatg cttatatatt ctacatctgt | 2101 |
| | aaagtgctga gttttatgga gagaggcctt tttatgcatt aaattgtaca tggcaaataa | 2161 |
| | atcccagaag gatctgtaga tgaggcacct gctttttctt ttctctcatt gtccacctta | 2221 |
| | ctaaaagtca gtagaatctt ctacctcata acttccttcc aaaggcagct cagaagatta | 2281 |
| | gaaccagact tactaaccaa ttccaccccc caccaacccc cttctactgc ctactttaaa | 2341 |
| | aaaattaata gttttctatg gaactgatct aagattagaa aaattaattt tctttaattt | 2401 |
| | cattatgaac ttttatttac atgactctaa gactataaga aaatctgatg gcagtgacaa | 2461 |
| | agtgctagca tttattgtta tctaataaag accttggagc atatgtgcaa cttatgagtg | 2521 |
| | tatcagttgt tgcatgtaat ttttgccttt gtttaagcct ggaacttgta agaaaatgaa | 2581 |
| | aatttaattt ttttttctag gacgagctat agaaaagcta ttgagagtat ctagttaatc | 2641 |
| | agtgcagtag ttggaaacct tgctggtgta tgtgatgtgc ttctgtgctt ttgaatgact | 2701 |
| | ttatcatcta gtctttgtct atttttcctt tgatgttcaa gtcctagtct ataggattgg | 2761 |
| | cagtttaaat gctttactcc cccttttaaa ataaatgatt aaaatgtgct ttgaaaaaaa | 2821 |
| | aaaaaaaaaa aaaaaaaaaa aaaa | |

Homo sapiens transcobalamin I (vitamin B12 binding protein, R binder family)
(TCN1), mRNA
NCBI Reference Sequence: NM_001062.3 (SEQ ID. NO. 27)

| | | |
|---|---|---|
| 1 | ggctgaggca acctgaagga ggagctctca ttaccttctg cccatcactt aataaatagc | |
| 61 | cagccaattc atcaacattc tggtacactg ttggagagat gagacagtca caccagctgc | 121 |
| | ccctagtggg gctcttactg ttttctttta ttccaagcca actatgcgag atttgtgagg | 181 |
| | taagtgaaga aaactacatc cgcctaaaac ctctgttgaa tacaatgatc cagtcaaact | 241 |
| | ataacagggg aaccagcgct gtcaatgttg tgttgtccct caaacttgtt ggaatccaga | 301 |
| | tccaaaccct gatgcaaaag atgatccaac aaatcaaata caatgtgaaa agcagattgt | 361 |
| | cagatgtaag ctcgggagag cttgccttga ttatactggc tttgggagta tgtcgtaacg | 421 |
| | ctgaggaaaa cttaatatat gattaccacc tgatcgacaa gctagaaaat aaattccaag | 481 |
| | cagaaattga aaatatggaa gcacacaatg gcactcccct gactaactac taccagctca | 541 |
| | gcctggacgt tttggccttg tgtctgttca atgggaacta ctcaaccgcc gaagttgtca | 601 |
| | accacttcac tcctgaaaat aaaaactatt attttggtag ccagttctca gtagatactg | 661 |
| | gtgcaatggc tgtcctggct ctgacctgtg tgaagaagag tctaataaat gggcagatca | 721 |
| | aagcagatga aggcagttta aagaacatca gtatttatac aaagtcactg gtagaaaaga | 781 |
| | ttctgtctga gaaaaaagaa aatggtctca ttggaaacac atttagcaca ggagaagcca | 841 |
| | tgcaggccct ctttgtatca tcagactatt ataatgaaaa tgactggaat tgccaacaaa | 901 |
| | ctctgaatac agtgctcacg gaaatttctc aaggagcatt cagcaatcca aacgctgcag | 961 |
| | cccaggtctt acctgccctg atgggaaaga ccttcttgga tattaacaaa gactcttctt | 1021 |
| | gcgtctctgc ttcaggtaac ttcaacatct ccgctgatga gcctataact gtgacacctc | 1081 |
| | ctgactcaca atcatatatc tccgtcaatt actctgtgag aatcaatgaa acatatttca | 1141 |
| | ccaatgtcac tgtgctaaat ggttctgtct tcctcagtgt gatggagaaa gcccagaaaa | 1201 |
| | tgaatgatac tatatttggt ttcacaatgg aggagcgctc atgggggccc tatatcacct | 1261 |
| | gtattcaggg cctatgtgcc aacaataatg acagaaccta ctgggaactt ctgagtggag | 1321 |
| | gcgaaccact gagccaagga gctggtagtt acgttgtccg caatggagaa aacttggagg | 1381 |
| | ttcgctggag caaatactaa taagcccaaa ctttcctcag ctgcataaaa tccatttgca | 1441 |
| | gtggagttcc atgtttattg tccttatgcc ttcttcttca tttatcccag tacgagcagg | 1501 |
| | agagttaata acctcccctt ctctctctac atgttcaata aaagttgttg aaagattaac | 1561 |
| | aactataaaa aaaaaaa | |

Homo sapiens visinin-like 1 (VSNL1), mRNA
NCBI Reference Sequence: NM_003385.4 (SEQ ID. NO. 28)

| | | |
|---|---|---|
| 1 | aggcggcttt tggtcacagg ctcccgagtt ctcctagctg gggctgcgga gctgggggga | |
| 61 | gggaagagag gaaaggggag ggggtgcctg gagaggcgga ggctcgcgcg cctgcgcatc | 121 |
| | cagctccagg gaccctaggt tttctatggg attcccaatc tgcagcagag atttacccga | 181 |
| | gcgtgttgcg gcagcggctg ggcttgcaag gcgcgatcca agagggattt aagcagccca | 241 |
| | gagctccaga gaaaaagaga gcgagagaga accacacaca gagacggctt aagcgtttac | 301 |
| | ccgaattaaa tatatatttt taaaaagaac tgttgagttt tatcattttc gttaagtgac | 361 |
| | cgtgcgcagc gctgtaactg caggatgggg aagcagaata gcaaactggc ccctgaagtg | 421 |
| | atggaggacc tggtgaagag cacagagttt aatgagcatg aactcaagca gtggtacaaa | 481 |
| | ggatttctca aggactgtcc aagtgggagg ctaaatctcg aggaatttca gcagctctat | 541 |
| | gtgaagttct ttccttatgg agacgcctcc aagtttgccc agcatgcctt ccgaaccttc | 601 |
| | gacaagaatg gggacggcac cattgacttc cgagagttca tctgcgctct gtccatcacc | 661 |
| | tccaggggca gctttgagca gaagctgaac tgggccttca atatgtatga cctggatggt | 721 |
| | gatggcaaga tcacccgagt ggagatgctg gagatcatcg aggctatcta caaaatggta | 781 |
| | ggcactgtga tcatgatgaa aatgaatgag gatggcctga cgcctgagca gcgagtagac | 841 |
| | aagattttca gcaagatgga taagaacaaa gatgaccaga ttacactgga tgaattcaaa | 901 |
| | gaagctgcaa agagcgaccc ttccattgta ttacttctgc agtgcgacat ccagaaatga | 961 |
| | gctgatgtca atgctatgga ctgcacaaaa gtctcaatgt tccattcagt ctgcagctat | 1021 |
| | tcacacacac acacacacac acacacacac acacacacac acacacaaat attgcttgga | 1081 |
| | ctacctataa atggacttgc ttcttgtgtt tgaaacactc gtgtgcatga gaatgtcatt | 1141 |
| | tgctaatgaa ttttaaaagc atatataaaa caaaacaaac aacctgccac aatgtgatat | 1201 |
| | gtgtaatatc atttcataaa aatccctctt cctccaaagc ctgggcagaa atgtgctgca | 1261 |
| | aagagttata tgacttcttg ttcatgtttt gctaatgctc gtatctcctt gattacataa | 1321 |
| | tgttagtagc actgagaccc ccatggtaat gtaacttaat tataagctat gtcactaccc | 1381 |
| | tcctgtaaaa tactattgga cagacacaga gggacccttg gctcctgtgt ctggtccaca | 1441 |
| | caccacagaa gcttgtatta tcagtgaata taaatgtact acatttgcat gccttttggg | 1501 |
| | tttgccttaa ttcttacctc atttgcatcc tatcgatctg gaaagagctg ttttggatga | 1561 |
| | atgcagtata aaatgtaaaa accctgctaa atgacttatt gattaagtat atctatctat | 1621 |
| | atatacatat acacaaagat attatttatc gaaagtaaaa aagatggaaq tgtattggtt | 1681 |
| | tctgtttgaa ttttcaaagg cttccaatgt ggtggcaata aatgtcccaa ataaatttat | 1741 |
| | aacaattgat tttcccccta attcttattt tataatttta aaattgcagc agttgctagc | 1801 |
| | aacaacttac taaatctact cttaaatata caactttgga atttgaagaa ttaatgacaa | 1861 |
| | caaaagggaa aaaagcaact ttccaacttt tcatccaggc tcccaaaaga gggacaacga | 1921 |
| | acatggcatg tgaaaagtaa aacagatttg ttcattccga aaaaaaaatg ttcattctat | 1981 |
| | gacaataaat tttatctcag tgtgaaaaaa aaaa | |

Homo sapiens ubiquitin D (UBD), mRNA
NCBI Reference Sequence: NM_006398.2 (SEQ ID. NO. 29)

| | | |
|---|---|---|
| 1 | gattgcttga ggagagaagt atgtgatcag aaagcattct ttgtctatta actcctgccc | |
| 61 | agcaaaagtg aaagaaaatt catgggagca tgcaagaaca aagagcacag caaagctgga | 121 |
| | caaacacagc aatccaggca ggggatttcc aactcaactc tggtatgtaa gctgcatgca | 181 |
| | aagtcctttt tctgtctctg gtttctggcc ccttgtctgc agagatggct cccaatgctt | 241 |
| | cctgcctctg tgtgcatgtc cgttccgagg aatgggattt aatgaccttt gatgccaacc | 301 |
| | catatgacag cgtgaaaaaa atcaaagaac atgtccggtc taagaccaag gttcctgtgc | 361 |
| | aggaccaggt tcttttgctg ggctccaaga tcttaaagcc acggagaagc ctctcatctt | 421 |
| | acggcattga caaagagaag accatccacc ttaccctgaa agtggtgaag cccagtgatg | 481 |
| | aggagctgcc cttgtttctt gtggagtcag gtgatgaggc aaagaggcac ctcctccagg | 541 |
| | tgcgaaggtc cagctcagtg gcacaagtga aagcaatgat cgagactaag acgggtataa | 601 |
| | tccctgagac ccagattgtg acttgcaatg gaaagagact ggaagatggg aagatgatgg | 661 |
| | cagattacgg catcagaaag ggcaacttac tcttcctggc atcttattgt attggagggt | 721 |
| | gaccaccctg ggcatggggt gttggcaggg gtcaaaaagc ttatttcttt taatctctta | 781 |
| | ctcaacgaac acatcttctg atgatttccc aaaattaatg agaatgagat gagtagagta | 841 |
| | agatttgggt gggatgggta ggatgaagta tattgcccaa ctctatgttt ctttgattct | 901 |
| | aacacaatta attaagtgac atgattttta ctaatgtatt actgagacta gtaaataaat | 961 |
| | ttttaagcca a | |

Homo sapiens absent in melanoma 2 (AIM2), mRNA
NCBI Reference Sequence: NM_004833.1 (SEQ ID. NO. 30)

| | | |
|---|---|---|
| 1 | tcagccaatt agagctccag ttgtcactcc tacccacact gggcctgggg gtgaagggaa | |
| 61 | gtgtttatta ggggtacatg tgaagccgtc cagaagtgtc agagtctttg tagctttgaa | 121 |
| | agtcacctag gttatttggg catgctctcc tgagtcctct gctagttaag ctctctgaaa | 181 |
| | agaaggtggc agacccggtt tgctgatcgc cccagggatc aggaggctga tcccaaagtt | 241 |
| | gtcagatgga gagtaaatac aaggagatac tcttgctaac aggcctggat aacatcactg | 301 |
| | atgaggaact ggataggttt aagttctttc tttcagacga gtttaatatt gccacaggca | 361 |
| | aactacatac tgcaaacaga atacaagtag ctaccttgat gattcaaaat gctggggcgg | 421 |
| | tgtctgcagt gatgaagacc attcgtattt ttcagaagtt gaattatatg cttttggcaa | 481 |
| | aacgtcttca ggaggagaag gagaaagttg ataagcaata caaatcggta acaaaaccaa | 541 |
| | agccactaag tcaagctgaa atgagtcctg ctgcatctgc agccbtcaga aatgatgtcg | 601 |
| | caaagcaacg tgctgcacca aaagtctctc ctcatgttaa gcctgaacag aaacagatgg | 661 |
| | tggcccagca ggaatctatc agagaagggt ttcagaagcg ctgtttgcca qttatggtac | 721 |
| | tgaaagcaaa gaagcccttc acgtttgaga cccaagaagg caagcaggag atgtttcatg | 781 |
| | ctacagtggc tacagaaaag gaattcttct ttgtaaaagt ttttaataca ctgctgaaag | 841 |
| | ataaattcat tccaaagaga ataattataa tagcaagata ttatcggcac agtggtttct | 901 |
| | tagaggtaaa tagcgcctca cgtgtgttag atgctgaatc tgaccaaaag gttaatgtcc | 961 |
| | cgctgaacat tatcagaaaa gctggtgaaa ccccgaagat caacacgctt caaactcagc | 1021 |
| | cccttggaac aattgtgaat ggtttgtttg tagtccagaa ggtaacagaa aagaagaaaa | 1081 |
| | acatattatt tgacctaagt gacaacactg ggaaaatgga agtactgggg gttagaaacg | 1141 |
| | aggacacaat gaaatgtaag gaaggagata aggttcgact tacattcttc acactgtcaa | 1201 |
| | aaaatggaga aaaactacag ctgacatctg gagttcatag caccataaag gttattaagg | 1261 |
| | ccaaaaaaaa aacatagaga agtaaaaagg accaattcaa gccaactggt ctaagcagca | 1321 |
| | tttaattgaa gaatatgtga tacagcctct tcaatcagat tgtaagttac ctgaaagctg | 1381 |
| | cagttcacag gctcctctct ccaccaaatt aggatagaat aattgctgga taaacaaatt | 1441 |
| | cagaatatca acagatgatc acaataaaca tctgtttctc attcc | |

Homo sapiens ATP-binding cassette, sub-family C (CFTR/MRP), member 9 (ABCC9),
transcript variant SUR2B, mRNA
NCBI Reference Sequence: NM_020297.1 (SEQ ID. NO. 31)

| | | |
|---|---|---|
| 1 | atgagccttt cattttgtgg taacaacatt tcttcatata atatcaacga tggtgtacta | |
| 61 | caaaattcct gctttgtgga tgccctcaac ctggtccctc atgtctttct gttgtttatc | 121 |
| | acttttccaa tattgtttat tgggtggggg agccaaagct caaaagtaca aattcaccac | 181 |
| | aacacatggc ttcattttcc gggacataac ctgagatgga ttcttacatt cgctctcctg | 241 |
| | tttgtgcatg tctgtgaaat agcagaaggc attgtttcag actcgcggcg ggaatcaagg | 301 |
| | cacctccacc tctttatgcc agccgtgatg ggattcgttg ccactacaac atcgatagtg | 361 |
| | tattatcata atatcgaaac atcaaatttt cctaaattac ttttagccct gttcctgtat | 421 |
| | tgggtaatgg cctttattac aaaaacaata aaattggtta agtactgtca gtctggcttg | 481 |
| | gacatatcaa acctgcgttt ctgcatcaca ggcatgatgg tcatcttgaa tgggctcttg | 541 |
| | atggctgtgg agatcaatgt cattcgagtc aggagatatg tatttttcat gaatcctcag | 601 |
| | aaagtaaagc ctcctgaaga cctccaggat ctgggagtga gatttcttca accatttgtg | 661 |
| | aatttgctgt caaaagcaac atactggtgg atgaacacac ttattatatc tgctcacaaa | 721 |
| | aagcctattg atctgaaggc aattggaaaa ttgccaatag caatgagagc agtaacaaat | 781 |
| | tatgtttgcc tgaaagatgc atatgaagaa caaaagaaaa aagttgcaga tcatccaaat | 841 |
| | cggactccat ctatatggct tgcaatgtac agagcttttg ggcgaccaat tctacttagt | 901 |
| | agcacattcc gctatctggc tgatttactg ggttttgctg gacctctttg tatttctgga | 961 |
| | atagttcagc gtgtgaatga aacccagaat gggacaaata acacaactgg aatttcagaa | 1021 |
| | accctctcat caaaggaatt tcttgaaaac gcttacgttc tagcagttct tctcttcttg | 1081 |
| | gctcttattc tgcaaaggac atttttgcag gcttcctact atgtaaccat agagactggc | 1141 |
| | attaacctcc gtggagctct gctggccatg atttataata aaatccttag gctctctacg | 1201 |
| | tctaacttat ccatggggga gatgactctg gggcagatca acaacttagt cgccattgaa | 1261 |
| | actaatcaac tcatgtggtt tttgttcctg tgtcccaatc tatgggctat gcctgttcag | 1321 |
| | atcataatgg gcgtgattct gctctataat ttacttggat caagtgcatt ggtcggtgca | 1381 |
| | gctgtcattg tgctccttgc gccaattcag tactttattg ctacaaagtt ggcagaggct | 1441 |
| | cagaaaagta cacttgatta ttccactgag agactcaaga aaacaaatga aatattgaaa | 1501 |
| | ggcatcaaac ttctaaaatt gtatgcctgg gaacacattt tctgcaaaag tgtggaggaa | 1561 |
| | acaagaatga aagaactatc tagtctcaaa acctttgcac tatatacatc actctccatc | 1621 |
| | ttcatgaatg cagcaattcc catagcagct gttcttgcta catttgtgac ccatgcgtat | 1681 |
| | gccagtggaa acaatctgaa acctgcagag gcctttgctt cactgtctct cttccatatc | 1741 |
| | ctggtcacac cactgtccct gctcttcacg gtggtcagat ttgcagtcaa agccatcata | 1801 |
| | agtgttcaaa agctgaatga gtttctcttg agtgatgaga ttggtgacga cagttggcga | 1861 |
| | actggtgaaa gttcgcttcc ttttgagtcc tgtaagaagc acactggagt tcagccaaaa | 1921 |
| | actataaaca ggaaacagcc tggaagatat cacctggaca gctatgagca atcaacacgg | 1981 |
| | cgtctacgtc ccgcagaaac agaggacatt gcaataaagg tcacaaatgg atacttttca | 2041 |
| | tggggcagtg gtttagctac attatccaat atagatattc gaattccaac aggtcagtta | 2101 |
| | accatgattg tgggccaagt aggatgtggg aagtcctctc ttctccttgc catcctcggt | 2161 |
| | gagatgcaga cattggaagg aaaagttcac tggagcaatg taaatgaatc tgagccttct | 2221 |
| | tttgaagcaa ccagaagtag gaacaggtac tctgtggcat atgcagctca aaagccttgg | 2281 |
| | ctattaaatg ctacagtaga agaaaatatt acttttggaa gtccttttaa caaacagagg | 2341 |
| | tacaaagctg tcacagatgc ctgttctctt cagccagata ttgacttatt accatttgga | 2401 |
| | gatcaaactg aaattggaga gaggggcatc aacctgagtg ggggacagag gcagagaatc | 2461 |
| | tgtgtggcac gagcgctgta tcaaaacacc aacattgtct ttttggatga tccattctca | 2521 |
| | gccctggaca ttcacttgag tgatcattta atgcaggagg ggattttgaa attcctgcaa | 2581 |
| | gatgacaaaa ggacactcgt tcttgtgact cacaaattac agtatctgac gcatgctgac | 2641 |
| | tggatcatag ccatgaaaga tggaagtgtc ctaagagaag gaactttgaa ggacattcaa | 2701 |
| | accaaagatg ttgagcttta tgaacactgg aaaacactta tgaatcggca agatcaagaa | 2761 |
| | ttagaaaagg atatggaagc tgaccaaact actttagaga ggaaaactct ccgacgggcc | 2821 |
| | atgtattcaa gagaagccaa agcccagatg gaggacgaag acgaagagga agaagaggag | 2881 |
| | gaagatgagg atgataacat gtccactgta atgaggctca ggactaaaat gccatggaaa | 2941 |
| | acctgctggc gctacctgac atctggagga ttcttcctgc tcatcctgat gattttctct | 3001 |
| | aagcttttga agcattcggt cattgtagct atagactatt ggctggccac atggacatcg | 3061 |
| | gagtacagta taaacaatac tggaaaagct gatcagacct actatgtggc tggctttagc | 3121 |
| | atactctgtg gagcaggcat tttcctttgc cttgttacat ccctcactgt agaatggatg | 3181 |
| | ggtctcacag ctgccaaaaa tcttcaccac aaccttctca ataagataat ccttggacca | 3241 |
| | ataaggtttt ttgataccac acccctggga ctgattctca atcgcttttc agctgatact | 3301 |
| | aatatcattg atcagcacat ccctccaacc ttggaatctc taactcgctc aacactgctc | 3361 |
| | tgcctgtctg ccattgggat gatttcttat gctactcctg tgttcctggt tgctctcctg | 3421 |
| | ccccttggtg ttgcctttta ttttatccag aaatactttc gggttgcctc taaggacctc | 3481 |
| | caggaactcg acgatagtac ccagctccct ctgctctgtc acttctcaga aacagcagaa | 3541 |
| | ggactcacca ccattcgggc ctttaggcat gaaaccagat ttaaacaacg tatgctggaa | 3601 |
| | ctgacggata caaacaacat tgcctactta tttctctcag ctgccaacag atggctggag | 3661 |
| | gtcaggacgg attatctggg agcttgcatt gtcctcactg catctatagc atccattagt | 3721 |
| | gggtcttcca attctggatt ggtaggcttg ggtcttctgt atgcacttac gataaccaat | 3781 |
| | tatttgaatt gggttgtgag gaacttggct gacctggagg tccagatggg tgcagtgaag | 3841 |
| | aaggtgaaca gtttcctgac tatggagtca gagaactatg aaggcacaat ggatccttct | 3901 |
| | caagttccag aacattggcc acaagaaggg gagatcaaga tacatgatct gtgtgtcaga | 3961 |
| | tatgaaaata atctgaaacc tgttcttaag cacgtcaagg cttacatcaa acctggacaa | 4021 |
| | aaggtgggca tatgtggtcg cactggcagt gggaaatcat cgttatctct ggctttcttc | 4081 |
| | agaatggttg atatatttga tggaaaaatt gtcattgatg ggatagacat ttccaaatta | 4141 |
| | ccactgcaca cactacgttc tagactttca atcattctgc aggatccaat actattcagt | 4201 |
| | ggttccatta gatttaattt agatccagag tgcaaatgca cagatgacag actctgggaa | 4261 |
| | gccttagaaa ttgctcagct gaagaatatg gtcaaatctc tacctggagg tctagatgcg | 4321 |
| | gttgtcactg aaggtgggga gaattttagc gtgggacaga gacagctatt ttgccttgcc | 4381 |
| | agggcctttg tccgcaaaag cagcattctt attatggatg aggcaacagc ttccattgac | 4441 |
| | atggccacag agaatatttt gcaaaaagta gtaatgacag cctttgcaga ccggaccgtg | 4501 |
| | gtgacaatgg ctcatcgagt acacactatt ctgagggcag acctggttat tgtgatgaag | 4561 |
| | cgaggaaata ttttagaata tgacactcca gaaagcctct tggctcagga aaatggagta | 4621 |
| | tttgcttctt ttgttcgcgc agacatgtga | |

Homo sapiens serpin peptidase inhibitor, glade B (ovalbumin), member 13
(SERPINB13), mRNA
NCBI Reference Sequence: NM_012397.2 (SEQ ID. NO. 32)

| | | |
|---|---|---|
| 1 | ctataaatta aggatcccag ctacttaatt gacttatgct tcctagttcg ttgcccagcc | |
| 61 | accaccgtct ctccaaaaac ccgaggtctc gctaaaatca tcatggattc acttggcgcc | 121 |
| | gtcagcactc gacttgggtt tgatcttttc aaagagctga agaaaacaaa tgatggcaac | 181 |
| | atcttctttt cccctgtggg catcttgact gcaattggca tggtcctcct ggggacccga | 241 |
| | ggagccaccg cttcccagtt ggaggaggtg tttcactctg aaaaagagac gaagagctca | 301 |
| | agaataaagg ctgaagaaaa agaggtgatt gagaacacag aagcagtaca tcaacaattc | 361 |
| | caaaagtttt tgactgaaat aagcaaactc actaatgatt atgaactgaa cataaccaac | 421 |
| | aggctgtttg gagaaaaaac atacctcttc cttcaaaaat acttagatta tgttgaaaaa | 481 |
| | tattatcatg catctctgga acctgttgat tttgtaaatg cagccgatga aagtcgaaag | 541 |
| | aagattaatt cctgggttga aagcaaaaca aatgaaaaaa tcaaggactt gttcccagat | 601 |
| | ggctctatta gtagctctac caagctggtg ctggtgaaca tggtttattt taaagggcaa | 661 |
| | tgggacaggg agtttaagaa agaaaatact aaggaagaga aattttggat gaataagagc | 721 |
| | acaagtaaat ctgtacagat gatgacacag agccattcct ttagcttcac tttcctggag | 781 |
| | gacttgcagg ccaaaattct agggattcca tataaaaaca acgacctaag catgtttgtg | 841 |
| | cttctgccca acgacatcga tggcctggag aagataatag ataaaataag tcctgagaaa | 901 |
| | ttggtagagt ggactagtcc agggcatatg gaagaaagaa aggtgaatct gcacttgccc | 961 |
| | cggtttgagg tggaggacgg ttacgatcta gaggcggtcc tggctgccat ggggatgggc | 1021 |
| | gatgccttca gtgagcacaa agccgactac tcgggaatgt cgtcaggctc cgggttgtac | 1081 |
| | gcccagaagt tcctgcacag ttcctttgtg gcagtaactg aggaaggcac cgaggctgca | 1141 |
| | gctgccaccg gcataggctt tactgtcaca tccgccccag gtcatgaaaa tgttcactgc | 1201 |
| | aatcatccct tcctgttctt catcaggcac aatgaatcca acagcatcct cttcttcggc | 1261 |
| | agattttctt ctccttaaga tgatcttttc catggcattg ctgcttttag caaaaaacaa | 1321 |
| | ctaccagtgt tactcatatg attatgaaaa tcgtccattc ttttaaatgt tgtctcactt | 1381 |
| | gcatttccag tcttggccat caaatcaatg atttaatgac tccaataatg tgtgtgttta | 1441 |
| | taaccatcct cgaaagtgaa atgtcctttt ttttgtgcca tgcgtaaggt gagtcaaacc | 1501 |
| | aaacctcatt gataatctcc cctttggttt cctttgaaag taaattggta tcttgtagtt | 1561 |
| | ttgtgcacac gaaaggagag aaagtttctc cagtaaagag tacgaactag taattttggg | 1621 |
| | gggtctctct aattctggta ttttgacatg ttataatacg caagtaaaat aaaacaatag | 1681 |
| | tttactcagc tcatgttact attccccaac agatattgtg gcaaatcaca cataggaaag | 1741 |
| | aggatttggg aatacagtag caaaacataa attaaaactc aaatgcccag gacaaaataa | 1801 |
| | aacaatatac cagatggaga ggatgcccgt attttcatct tccattctaa cattatccat | 1861 |
| | tgttagatgc ataagcattt tgatattgtg taataaatgt ggtatttgag aagataaatg | 1921 |
| | atgtagttga tcagtaatcc tcctctatca cctttttaga ctttgtaagg taaatatttg | 1981 |
| | gactaacttt tagaaaagtt tccctttttt tctccattta catttttctg gttttttttt | 2041 |
| | ttttttttga gtgaggtacg agtattacca aatgatattt tctgaagatg ctttttggaa | 2101 |
| | agctctgaat ctatacctaa tgctcttaat tattggcttg tttcattttt ttcctccagt | 2161 |
| | ttttaacaag atcacataac tggcttattt ttaacagctt tgtcaaacta caatttacat | 2221 |
| | gccgtaaaat gtacacactg taattttata attcattgac ttttagtaaa ttttctagcg | 2281 |
| | ttatgcatcg ccacaatcca gttttagaat atttccatga ccctaagaag tttcctcatg | 2341 |
| | tctattaata ttcccaatcc taggcaccac tgagttgttt tctgtcttta taagtttttc | 2401 |
| | tttctacatc ttatataaat ggaatcataa tacatgtagt attttgtgtc tggcgtcttg | 2461 |
| | cacttagcat ggtgttcttg aggttcatct gttgtagtat gtattgatac ttaggatttt | 2521 |
| | tttattgccg aatactattc cattgcatgg aaaagaccta ttttatttct aggttcacca | 2581 |
| | gttgagggac atttggattg ttcccacttc ttgggctgtt aggaataatg ttgctctgaa | 2641 |
| | catgtaaata aagatctttg tgttcacata tgtttttcat tttctgttgg ggagattccc | 2701 |
| | taggctagaa attgctgggc catatgaaaa atcaatagtt agctttgtaa gaaacagtca | 2761 |
| | aactgttttc ccaacgtgac attttatatt cccaccagga atgtttaaaa ctagtgtctt | 2821 |
| | caaatcctca ccaacatcca ggattgtgtc tttatgatta tagccatttt tgtaggtaca | 2881 |
| | aagtggcatc tcatggtggt tttaatttgc atttccataa tatctaatta ggttgagctt | 2941 |
| | tttttatgtg cttattggcc atttgtttga ctttgtttgg tgaaatgtat acaaatcatt | 3001 |
| | tgctcatttt taatttgggt tgtctgtctt gtcttctcat tttattgagt taaatgagtt | 3061 |
| | cttaataatc tctggcttac aagtccttaa tttatcaaat atatgatacg tggacatttc | 3121 |
| | ctcataaaaa aaaaaaaaaa aaa | |

Homo sapiens indoleamine-pyrrole 2,3 dioxygenase (INDO), mRNA
NCBI Reference Sequence: NM_002164.3 (SEQ ID. NO. 33)

| | | |
|---|---|---|
| 1 | aatttctcac tgcccctgtg ataaactgtg gtcactggct gtggcagcaa ctattataag | |
| 61 | atgctctgaa aactcttcag acactgaggg gcaccagagg agcagactac aagaatggca | 121 |
| | cacgctatgg aaaactcctg gacaatcagt aaagagtacc atattgatga agaagtgggc | 181 |
| | tttgctctgc caaatccaca ggaaaatcta cctgattttt ataatgactg gatgttcatt | 241 |
| | gctaaacatc tgcctgatct catagagtct ggccagcttc gagaaagagt tgagaagtta | 301 |
| | aacatgctca gcattgatca tctcacagac cacaagtcac agcgccttgc acgtctagtt | 361 |
| | ctgggatgca tcaccatggc atatgtgtgg ggcaaaggtc atggagatgt ccgtaaggtc | 421 |
| | ttgccaagaa atattgctgt tccttactgc caactctcca agaaactgga actgcctcct | 481 |
| | attttggttt atgcagactg tgtcttggca aactggaaga aaaaggatcc taataagccc | 541 |
| | ctgacttatg agaacatgga cgttttgttc tcatttcgtg atggagactg cagtaaagga | 601 |
| | ttcttcctgg tctctctatt ggtggaaata gcagctgctt ctgcaatcaa agtaattcct | 661 |
| | actgtattca aggcaatgca aatgcaagaa cgggacactt tgctaaaggc gctgttggaa | 721 |
| | atagcttctt gcttggagaa agcccttcaa gtgtttcacc aaatccacga tcatgtgaac | 781 |
| | ccaaaagcat ttttcagtgt tcttcgcata tatttgtctg gctggaaagg caacccccag | 841 |
| | ctatcagacg gtctggtgta tgaagggttc tgggaagacc caaaggagtt tgcagggggc | 901 |
| | agtgcaggcc aaagcagcgt ctttcagtgc tttgacgtcc tgctgggcat ccagcagact | 961 |
| | gctggtggag gacatgctgc tcagttcctc caggacatga gaagatatat gccaccagct | 1021 |
| | cacaggaact tcctgtgctc attagagtca aatccctcag tccgtgagtt tgtcctttca | 1081 |
| | aaaggtgatg ctggcctgcg ggaagcttat gacgcctgtg tgaaagctct ggtctccctg | 1141 |
| | aggagctacc atctgcaaat cgtgactaag tacatcctga ttcctgcaag ccagcagcca | 1201 |
| | aaggagaata agacctctga agacccttca aaactggaag ccaaaggaac tggaggcact | 1261 |
| | gatttaatga atttcctgaa gactgtaaga agtacaactg agaaatccct tttgaaggaa | 1321 |
| | ggttaatgta acccaacaag agcacatttt atcatagcag agacatctgt atgcattcct | 1381 |
| | gtcattaccc attgtaacag agccacaaac taatactatg caatgtttta ccaataatgc | 1441 |
| | aatacaaaag acctcaaaat acctgtgcat ttcttgtagg aaaacaacaa aaggtaatta | 1501 |
| | tgtgtaatta tactagaagt tttgtaatct gtatcttatc attggaataa aatgacattc | 1561 |
| | aataaataaa aaaaaaaaaa aaaaaaaaaa aaaaaaaaaa aaaaaaaaaa aaaaaaaaaa | 1621 |
| | aaaaaaaaaa aaaaaaaaaa aaaaaaaaaa aa | |

Homo sapiens keratin 5 (KRT5), mRNA
NCBI Reference Sequence: NM_000424.3 (SEQ ID. NO. 34)

| | | |
|---|---|---|
| 1 | tcgacagctc tctcgcccag cccagttctg gaagggataa aaagggggca tcaccgttcc | |
| 61 | tgggtaacag agccaccttc tgcgtcctgc tgagctctgt tctctccagc acctcccaac | 121 |
| | ccactagtgc ctggttctct tgctccacca ggaacaagcc accatgtctc gccagtcaag | 181 |
| | tgtgtccttc cggagcgggg gcagtcgtag cttcagcacc gcctctgcca tcaccccgtc | 241 |
| | tgtctcccgc accagcttca cctccgtgtc ccggtccggg ggtggcggtg gtggtggctt | 301 |
| | cggcagggtc agccttgcgg gtgcttgtgg agtgggtggc tatggcagcc ggagcctcta | 361 |
| | caacctgggg ggctccaaga ggatatccat cagcactagt ggtggcagct tcaggaaccg | 421 |
| | gtttggtgct ggtgctggag gcggctatgg ctttggaggt ggtgccggta gtggatttgg | 481 |
| | tttcggcggt ggagctggtg gtggctttgg gctcggtggc ggagctggct ttggaggtgg | 541 |
| | cttcggtggc cctggctttc ctgtctgccc tcctggaggt atccaagagg tcactgtcaa | 601 |
| | ccagagtctc ctgactcccc tcaacctgca aatcgacccc agcatccaga gggtgaggac | 661 |
| | cgaggagcgc gagcagatca agaccctcaa caataagttt gcctccttca tcgacaaggt | 721 |
| | gcggttcctg gagcagcaga acaaggttct ggacaccaag tggaccctgc tgcaggagca | 781 |
| | gggcaccaag actgtgaggc agaacctgga gccgttgttc gagcagtaca tcaacaacct | 841 |
| | caggaggcag ctggacagca tcgtggggga acggggccgc ctggactcag agctgagaaa | 901 |
| | catgcaggac ctggtggaag acttcaagaa caagtatgag gatgaaatca acaagcgtac | 961 |
| | cactgctgag aatgagtttg tgatgctgaa gaaggatgta gatgctgcct acatgaacaa | 1021 |
| | ggtggagctg gaggccaagg ttgatgcact gatggatgag attaacttca tgaagatgtt | 1081 |
| | ctttgatgcg gagctgtccc agatgcagac gcatgtctct gacacctcag tggtcctctc | 1141 |
| | catggacaac aaccgcaacc tggacctgga tagcatcatc gctgaggtca aggcccagta | 1201 |
| | tgaggagatt gccaaccgca gccggacaga agccgagtcc tggtatcaga ccaagtatga | 1261 |
| | ggagctgcag cagacagctg gccggcatgg cgatgacctc cgcaacacca agcatgagat | 1321 |
| | ctctgagatg aaccggatga tccagaggct gagagccgag attgacaatg tcaagaaaca | 1381 |
| | gtgcgccaat ctgcagaacg ccattgcgga tgccgagcag cgtggggagc tggccctcaa | 1441 |
| | ggatgccagg aacaagctgg ccgagctgga ggaggccctg cagaaggcca agcaggacat | 1501 |
| | ggcccggctg ctgcgtgagt accaggagct catgaacacc aagctggccc tggacgtgga | 1561 |
| | gatcgccact taccgcaagc tgctggaggg cgaggaatgc agactcagtg gagaaggagt | 1621 |
| | tggaccagtc aacatctctg ttgtcacaag cagtgtttcc tctggatatg gcagtggcag | 1681 |
| | tggctatggc ggtggcctcg gtggaggtct tggcggcggc ctcggtggag gtcttgccgg | 1741 |
| | aggtagcagt ggaagctact actccagcag cagtgggggt gtcggcctag gtggtgggct | 1801 |
| | cagtgtgggg ggctctggct tcagtgcaag cagtggccga gggctggggg tgggctttgg | 1861 |
| | cagtggcggg ggtagcagct ccagcgtcaa atttgtctcc accacctcct cctcccggaa | 1921 |
| | gagcttcaag agctaagaac ctgctgcaag tcactgcctt ccaagtgcag caacccagcc | 1981 |
| | catggagatt gcctcttcta ggcagttgct caagccatgt tttatccttt tctggagagt | 2041 |
| | agtctagacc aagccaattg cagaaccaca ttctttggtt cccaggagag ccccattccc | 2101 |
| | agcccctggt ctcccgtgcc gcagttctat attctgcttc aaatcagcct tcaggtttcc | 2161 |
| | cacagcatgg cccctgctga cacgagaacc caaagttttc ccaaatctaa atcatcaaaa | 2221 |
| | cagaatcccc accccaatcc caaattttgt tttggttcta actacctcca gaatgtgttc | 2281 |
| | aataaaatgc ttttataata taaaaaaaaa aaaaaaaaaa | |

PREDICTED: Homo sapiens hypothetical LOC100130897 (LOC100130897), mRNA
NCBI Reference Sequence: XM_001718498.1 (SEQ ID. NO. 35)

| | | |
|---|---|---|
| 1 | atgacaccga ctcttttgct cacggtgact gtcccgaggg cggcgggtag cgccgggcag | |
| 61 | cgccgggctc cagggctccc gcgctccagt ggcccagcct gggcggagag cagagcgcgg | 121 |
| | cccccgcggc cccgcggcct cgagccccgg cacccccctg gctccccggc cctgcgcccc | 181 |
| | accgaccgca cgtgctcctc ctcctcggcg ggagtaggcg gcggggtcgg aggagcgcag | 241 |
| | ccggggtcgg tcccgctggg ccagcacctc gctcttgagc ggggacgaac tctcggacac | 301 |
| | gggcgtgtcg gccggcgaga tccgcctccg cttggcctgc tcgtaaatcc ccgagtcgct | 361 |
| | ggagtcgatg gacttgatcg aggagggcgt ctcgatccag ctggaatcgg acaggtcctt | 421 |
| | gggcttggcg tcctcggcgg cgccgggcgg cagcggcgag cgctcggcgg ccaggccctc | 481 |
| | ggcctcctcg cccaggtagg gattggcgcc ggccatgcgc gcggcggccg cggcgctgtt | 541 |
| | gggccagcag ggcagcaccg agcccgactt ggtgccgcag tactgcgggg gactgcgggc | 601 |
| | gccccagccc gacgggtcgg cgtagtagcc gagcgggcgg ccagtgcagc ctgcagcctg | 661 |
| | cagcggcagc gccttcacgc ccgccgccgc gtaagagagc agcgtggccg cgttgcccgc | 721 |
| | gaagtccgtg gccgtgtcat aggccgaggc cgcgaagtcc agccggttgt tggccggcgt | 781 |
| | cacaaaccag cgttgcggcg agggcgcgcc cgggtcctcg gcctgctgcg gcgacagcag | 841 |
| | cccgttggtg tgcggcacgc tgcggtccgt acccggcccg gggcccgcgc ccgcgcccgg | 901 |
| | gtggaagcgg gccttggcgt agttgctcac gaactggtcc tgcaggaaag agccggccat | 961 |
| | ggcgtagcgg gccccgggca cgatctgcga gcgcggcgag tcgttgggcg agggggtcag | 1021 |
| | gcggtccatg tcacagccgg tgtagatcct gcgggattgt ttcagaaacc cctaggaaaa | 1081 |
| | agccgcgcac gcagtaatca tgaaagactg gccttcaccc gtgttctgga acccgaggtt | 1141 |
| | tgctgctgga agcctccaaa gtacttagtg tctattgttt cccctgtgtg aaactttcac | 1201 |
| | tcccacctct actaatacaa acaagaatta ctactctgaa | |

Homo sapiens keratin 14 (epidermolysis bullosa simplex, Dowling-Meara,
Koebner) (KRT14), mRNA
NCBI Reference Sequence: NM_000526.3 (SEQ ID. NO. 36)

| | | |
|---|---|---|
| 1 | acccgagcac cttctcttca ctcagccaac tgctcgctcg ctcacctccc tcctctgcac | |
| 61 | catgactacc tgcagccgcc agttcacctc ctccagctcc atgaagggct cctgcggcat | 121 |
| | cgggggcggc atcgggggcg gctccagccg catctcctcc gtcctggccg gagggtcctg | 181 |
| | ccgcgccccc agcacctacg ggggcggcct gtctgtctca tcctcccgct tctcctctgg | 241 |
| | gggagcctac gggctggggg gcggctatgg cggtggcttc agcagcagca gcagcagctt | 301 |
| | tggtagtggc tttgggggag gatatggtgg tggccttggt gctggcttgg gtggtggctt | 361 |
| | tggtggtggc tttgctggtg gtgatgggct tctggtgggc agtgagaagg tgaccatgca | 421 |
| | gaacctcaat gaccgcctgg cctcctacct ggacaaggtg cgtgctctgg aggaggccaa | 481 |
| | cgccgacctg gaagtgaaga tccgtgactg gtaccagagg cagcggcctg ctgagatcaa | 541 |
| | agactacagt ccctacttca agaccattga ggacctgagg aacaagattc tcacagccac | 601 |
| | agtggacaat gccaatgtcc ttctgcagat tgacaatgcc cgtctggccg cggatgactt | 661 |
| | ccgcaccaag tatgagacag agttgaacct gcgcatgagt gtggaagccg acatcaatgg | 721 |
| | cctgcgcagg gtgctggacg aactgaccct ggccagagct gacctggaga tgcagattga | 781 |
| | gagcctgaag gaggagctgg cctacctgaa gaagaaccac gaggaggaga tgaatgccct | 841 |
| | gagaggccag gtgggtggag atgtcaatgt ggagatggac gctgcacctg gcgtggacct | 901 |
| | gagccgcatt ctgaacgaga tgcgtgacca gtatgagaag atggcagaga agaaccgcaa | 961 |
| | ggatgccgag gaatggttct tcaccaagac agaggagctg aaccgcgagg tggccaccaa | 1021 |
| | cagcgagctg gtgcagagcg gcaagagcga gatctcggag ctccggcgca ccatgcagaa | 1081 |
| | cctggagatt gagctgcagt cccagctcag catgaaagca tccctggaga acagcctgga | 1141 |
| | ggagaccaaa ggtcgctact gcatgcagct ggcccagatc caggagatga ttggcagcgt | 1201 |
| | ggaggagcag ctggcccagc tccgctgcga gatggagcag cagaaccagg agtacaagat | 1261 |
| | cctgctggac gtgaagacgc ggctggagca ggagatcgcc acctaccgcc gcctgctgga | 1321 |
| | gggcgaggac gcccacctct cctcctccca gttctcctct ggatcgcagt catccagaga | 1381 |
| | tgtgacctcc tccagccgcc aaatccgcac caaggtcatg gatgtgcacg atggcaaggt | 1441 |
| | ggtgtccacc cacgagcagg tccttcgcac caagaactga ggctgcccag ccccgctcag | 1501 |
| | gcctaggagg ccccccgtgt ggacacagat cccactggaa gatcccctct cctgcccaag | 1561 |
| | cacttcacag ctggaccctg cttcaccctc accccctcct ggcaatcaat acagcttcat | 1621 |
| | tatctgagtt gcat | |

Homo sapiens family with sequence similarity 83, member A (FAM83A),
transcript variant 1, mRNA
NCBI Reference Sequence: NM_032899.4 (SEQ ID. NO. 37)

| | | |
|---|---|---|
| 1 | ggaaagccgg ctcaccttcg cctccccctg cggctgggag gagaggaaat atcccatggc | |
| 61 | tgactgtgcc aaggaggtgt ctgagccagc cctcccggcc cgagggcagg gcaggtggcc | 121 |
| | ctgagagata agccaatccc gcagctgcag atgaggagtt ctgagaagca ttgctcagga | 181 |
| | cagcggtaaa tcacttcttg gaggtgccct gcacgccggt cctgggagca ggcggcctcc | 241 |
| | cgggggtgcg ggagccccac tcctccgtgg tgtgttccat ttgcttccca catctggagg | 301 |
| | agctgacgtg ccagcctccc ccagcaccac ccagggacgg gaggcatgag ccggtcaagg | 361 |
| | cacctgggca aaatccggaa gcgtctggaa gatgtcaaga gccagtgggt ccggccagcc | 421 |
| | agggctgact ttagtgacaa cgagagtgcc cggctggcca cggacgccct cttggatggg | 481 |
| | ggttctgaag cctactggcg ggtgctcagc caggaaggcg aggtggactt cttgtcctcg | 541 |
| | gtggaggccc agtacatcca ggcccaggcc agggagcccc cgtgtccccc agacaccctg | 601 |
| | ggaggggcgg aagcaggccc taagggactg gactccagct ccctacagtc cggcacctac | 661 |
| | ttccctgtgg cctcagaggg cagcgagccg gccctactgc acagctgggc ctcagctgag | 721 |
| | aagccctacc tgaaggaaaa atccagcgcc actgtgtact tccagaccgt caagcacaac | 781 |
| | aacatcagag acctcgtccg ccgctgcatc acccggacta gccaggtcct ggtcatcctg | 841 |
| | atggatgtgt tcacggatgt ggagatcttc tgtgacattc tagaggcagc caacaagcgt | 901 |
| | ggggtgttcg tttgtgtgct cctggaccag ggaggtgtga agctcttcca ggagatgtgt | 961 |
| | gacaaagtcc agatctctga cagtcacctc aagaacattt ccatccggag tgtggaagga | 1021 |
| | gagatatact gtgccaagtc aggcaggaaa ttcgctggcc aaatccggga gaagttcatc | 1081 |
| | atctcggact ggagatttgt cctgtctgga tcttacagct tcacctggct ctgcggacac | 1141 |
| | gtgcaccgga acatcctctc caagttcaca ggccaggcgg tggagctgtt tgacgaggag | 1201 |
| | ttccgccacc tctacgcctc ctccaagcct gtgatgggcc tgaagtcccc gcggctggtc | 1261 |
| | gcccccgtcc cgcccggagc agccccggcc aatggccgcc ttagcagcag cagtggctcc | 1321 |
| | gccagtgacc gcacgtcctc caaccccttc agcggccgct cggcaggcag ccaccccggt | 1381 |
| | acccgaagtg tgtccgcgtc ttcagggccc tgtagccccg cggccccaca cccgcctcca | 1441 |
| | ccgccccggt tccagcccca ccaaggccct tggggagccc cgagtcccca ggcccacctc | 1501 |
| | tccccgcggc cccacgacgg cccgcccgcc gctgtctaca gcaacctggg ggcctacagg | 1561 |
| | cccacgcggc tgcagctgga gcagctgggc ctggtgccga ggctgactcc aacctggagg | 1621 |
| | cccttcctgc aggcctcccc tcacttctga aggtcccatc ccctgctgcc ctccgcaggc | 1681 |
| | ccagggctgg gcactccctg agacccaaag acccacctca acgacgagtg gcgttgagcc | 1741 |
| | acttcccttt gaaaagacac tcaaaatcac tgccatggtt caatgttccc aggccccagg | 1801 |
| | ccatccactt gccggccccc accagttctt gggttccccg ctctagtttg acctgtgcag | 1861 |
| | cacattccag aaggttccag ggaggttgtg gggcagctag aggacaaaat catgaaaaca | 1921 |
| | gagtccctgt cttccagaga tcatccgggg ctttaatatt aatggccccc aaaactccgt | 1981 |
| | aagaagcagg aaatgcagcc caagttttac aaatgggtaa acagaggcac tgagagatag | 2041 |
| | atggtagttt ggtacttctg gttcccagtg cccaggaatg gtccactccc aagaaattca | 2101 |
| | ggaaagaaag actgaggaga aggtgtggga acattctgga tgtttcggga gagttgggga | 2161 |
| | aactcctcct cttaggaaag gctaatacta gggtatcctt gggcccaatg aattaggggt | 2221 |
| | gaggccccag aacccgttat ctatgagttg tatgggggag ccatctgaag ctgtagccac | 2281 |
| | cagggatgca gctagctgag gagtttgggg tgttgggttg gacaaggcag gttagtagac | 2341 |
| | tcagattctt gcttcaaaga gccttgggct ggcctggagg tccctggagt ctagactgga | 2401 |
| | cctaggagct tgagttgtca ggggccagga ctggccccac tgcagtgccc aggccagtct | 2461 |
| | tgagcagcag ggagggctca gctgtcccca gatccaggtg cctctgacca gcctggtcac | 2521 |
| | ctcctgagga ataaatgctg aacctcacaa gccccatcat tcatttcttc tcaattcaca | 2581 |
| | gtgcccctct ttgtttctgg ggtggaacta ggtcctgagg gcacagccta gctgagtgca | 2641 |
| | aagaaatata ggatgcttag aaagcataca ggaggggcca ggcgtggtgg ctcatgcctg | 2701 |
| | taatcccaga actttgggat gccaaggtgg ttggattacc tgagatcagg tggattacct | 2761 |
| | ggtctcgaga ccagcctgac caatatggtg aaaccccgtc tctactaaaa atacaaaaat | 2821 |
| | taggctgaga caggagaatt gcttgaaccc aggaagcaga ggttgcaatg agctgagatt | 2881 |
| | gcatcactgc actccagcat gggcaacaaa gcaagactcc gtcacagaaa aaaaaaaaaa | 2941 |
| | aaaaaa | |

Homo sapiens family with sequence similarity 181, member B (FAM181B), mRNA.
NCBI Reference Sequence: NM_175885.3 (SEQ ID. NO. 38)

| | | |
|---|---|---|
| 1 | agcgcagcga gccaggcccg gaactagtag gctgcgccgc gcgcgccgcg ccggggcggg | |
| 61 | agctgggtct gggcggcggg caggagctgg cgggggcgca cgggcagcgc tgcggacagc | 121 |
| | ccgggagccg cggcgatggc ggtgcaggcg gcgctcctca gcacgcaccc tttcgtgccc | 181 |
| | ttcggcttcg ggggctcccc ggacgggcta gggggcgcct tcggagccct ggacaagggc | 241 |
| | tgctgtttcg aggacgatga gaccggggct ccggcgggtg cgctgctgtc gggagccgaa | 301 |
| | ggaggggacg tgcgcgaggc cacccgcgat ctactcagct tcattgactc ggcgtccagc | 361 |
| | aacatcaagc tggcgctgga caagccgggc aagtcgaagc ggaaggtgaa ccaccgcaag | 421 |
| | tacctgcaga agcagatcaa gcgctgcagc ggcctcatgg gcgccgcgcc ccccggcccg | 481 |
| | ccctccccga gcgccgccga cacgccagcc aaacggccgc tggccgcccc tagcgccccg | 541 |
| | acagtcgcgg ccccggccca cggcaaggct gccccccggc gggaggcgtc gcaggccgcc | 601 |
| | gcggccgcca gcttgcaaag ccgaagtctg gccgcgctct tcgactcgct gcgccacgtc | 661 |
| | cccgggggtg ccgagccggc ggggggtgag gtggctgcgc cggcggccgg gctaggaggt | 721 |
| | gcgggcactg ggggcgcggg aggggacgtg gcaggccccg cgggggccac ggcgatccca | 781 |
| | ggggccagga aggtcccgct gcgggcacgc aatctgcctc cgtccttctt cacggagccg | 841 |
| | tcccgggcag gcggcggcgg gtgtggcccg tcggggccgg acgtgagctt gggcgacctg | 901 |
| | gagaagggcg cggaggccgt ggagttcttt gagctgctgg ggcccgacta cggcgccggc | 961 |
| | acggaggcgg cagtcttgct tgccgccgag cctctcgacg tgttccccgc cggagcctcc | 1021 |
| | gtactgcggg gacccccgga gctggagccc ggcctctttg agccgccgcc ggcagtggtg | 1081 |
| | ggaaacctac tgtaccccga gccctggagc gtcccgggct gctccccgac caaaaagagc | 1141 |
| | cccctgactg ccccccgcgg cggcttgacc ttgaacgagc ccttgagccc cctgtacccc | 1201 |
| | gccgctgcgg attctcccgg cggggaggac gggcggggcc atttggcctc tttcgccccc | 1261 |
| | ttctttccag actgcgccct gcccccgccg ccgccgcccc atcaggtgtc ctacgattac | 1321 |
| | agcgcgggct acagccgcac cgcctattcc agcctttgga gatccgacgg ggtttgggaa | 1381 |
| | ggggcgcggg gggaggaggg ggcgcaccgg gactgacttc gaggcacgct tcccttcatt | 1441 |
| | agagacggct gtggagagcg ccgcgcctcc gtgggtttct cctaaatctg aagaacgatg | 1501 |
| | ggaaaatgca cgtggagatg aaaccagatt tttaaaaatt caattaataa aagcaacttc | 1561 |
| | agaaaaaaga gatgaagacg agttggggat tgtttaatca caacctcaag tgttaaaaca | 1621 |
| | aaaacaaaca aacacgtttg taggttctta ctggaccaga ggagtcaaga aaccaagatg | 1681 |
| | gtttggggta tggggtgggg acggcaaaag gggtaagagc tggcttctgt agccacctgt | 1741 |
| | cccttctatt tttcagcgaa ggtcagtgta tttagtgtaa ttaccccttc taaacagtgt | 1801 |
| | cctagtccct cccttccctc tccttgagtg cattttgaat taaagcctat attgaaaaga | 1861 |
| | aaaaaaaaaa aaaaa | |

RST24587 Athersys RAGE Library Homo sapiens cDNA, mRNA sequence
GenBank: BG205162.1 (SEQ ID. NO. 39)
SEQUENCE
TTTCCGAGGCGTNCCTNCTNCCCTTTTNACCTCGGGACTCANCGTCTTCCTCACAGCACT
TCCATGTCATCTGCCCCGTGAAATCAGCCTAACGCCGTTTCTCAATGACGTGGATCGCCC
TAGGCCACCGCAACCTTCCGGAAGCTCTCTCAGCTCAGTTCCCATTCTCCCACCATCTCT
TGGTTCTCCTTCTACCTCACCGGTTGCTAGTCCTCCGCTTCGCAGCTGAAAATGTGCCCG
GGGCCTACTGTGGGCCTAGCCAGGCCTGCTTACGCAGTGCGGTTTCCCATGAATGATGCC
CAGTCATTATCACATAACCTGTGGCAAGCCAGCAAGATGGCCCTGGTGACAGCAAAAGAA
ACTGCACTAGGACCTGAATGTAGATCTCAGTCATGTTCCTTACTAACAGCACGTTTTGCA
ACCATGCGTTAAAGAAACATCTGACTCACAACAAAATTTTAAAGGGTTTATTTGAGTGAA
AAGCAATTTATGAATTGGGGAACACCTGACTGAAAGAGCGTTAGTATTCCAGAGACAAAA
CATCAAGTGCAAGTTTTTATTGGGAAAATGTAGAAGCACAATAAAGAAATTATTTTGATT
GGTTAAAAAAAAAAAAA
Homo sapiens granzyme B (granzyme 2, cytotoxic T-lymphocyte-associated serine
esterase 1) (GZMB), mRNA
NCBI Reference Sequence: NM_004131.3 (SEQ ID. NO. 40)

| | | |
|---|---|---|
| 1 | ccaagagcta aaagagagta agggggaaac aacagcagct ccaaccaggg cagccttcct | |
| 61 | gagaagatgc aaccaatcct gcttctgctg gccttcctcc tgctgcccag ggcagatgca | 121 |
| | ggggagatca tcgggggaca tgaggccaag ccccactccc gcccctacat ggcttatctt | 181 |
| | atgatctggg atcagaagtc tctgaagagg tgcggtggct tcctgataca agacgacttc | 241 |
| | gtgctgacag ctgctcactg ttggggaagc tccataaatg tcaccttggg ggcccacaat | 301 |
| | atcaaagaac aggagccgac ccagcagttt atccctgtga aaagacccat cccccatcca | 361 |
| | gcctataatc ctaagaactt ctccaacgac atcatgctac tgcagctgga gagaaaggcc | 421 |
| | aagcggacca gagctgtgca gcccctcagg ctacctagca acaaggccca ggtgaagcca | 481 |
| | gggcagacat gcagtgtggc cggctggggg cagacggccc ccctgggaaa acactcacac | 541 |
| | acactacaag aggtgaagat gacagtgcag gaagatcgaa agtgcgaatc tgacttacgc | 601 |
| | cattattacg acagtaccat tgagttgtgc gtgggggacc cagagattaa aaagacttcc | 661 |
| | tttaaggggg actctggagg ccctcttgtg tgtaacaagg tggcccaggg cattgtctcc | 721 |
| | tatggacgaa acaatggcat gcctccacga gcctgcacca aagtctcaag ctttgtacac | 781 |
| | tggataaaga aaaccatgaa acgctactaa ctacaggaag caaactaagc ccccgctgta | 841 |
| | atgaaacacc ttctctggag ccaagtccag atttacactg ggagaggtgc cagcaactga | 901 |
| | ataaatacct cttagctgag tggaaaaaaa aaaaaaaaaa aaaaaaaaaa aaaaa | |

DSG3 (SEQ ID. NO. 41)

| | | |
|---|---|---|
| 1 | aaagcagcag agacgctgca gagggctttt cttagacatc aactgcagac ggctggcagg | |
| 61 | atagaagcag cggctcactt ggactttttc accagggaaa tcagagacaa tgatggggct | 121 |
| | cttccccaga actacagggg ctctggccat cttcgtggtg gtcatattgg ttcatggaga | 181 |
| | attgcgaata gagactaaag gtcaatatga tgaagaagag atgactatgc aacaagctaa | 241 |
| | aagaaggcaa aaacgtgaat gggtgaaatt tgccaaaccc tgcagagaag gagaagataa | 301 |
| | ctcaaaaaga aacccaattg ccaagattac ttcagattac caagcaaccc agaaaatcac | 361 |
| | ctaccgaatc tctggagtgg gaatcgatca gccgcctttt ggaatctttg ttgttgacaa | 421 |
| | aaacactgga gatattaaca taacagctat agtcgaccgg gaggaaactc caagcttcct | 481 |
| | gatcacatgt cgggctctaa atgcccaagg actagatgta gagaaaccac ttatactaac | 541 |
| | ggttaaaatt ttggatatta atgataatcc tccagtattt tcacaacaaa ttttcatggg | 601 |
| | tgaaattgaa gaaaatagtg cctcaaactc actggtgatg atactaaatg ccacagatgc | 661 |
| | agatgaacca aaccacttga attctaaaat tgccttcaaa attgtctctc aggaaccagc | 721 |
| | aggcacaccc atgttcctcc taagcagaaa cactggggaa gtccgtactt tgaccaattc | 781 |
| | tcttgaccga gagcaagcta gcagctatcg tctggttgtg agtggtgcag acaaagatgg | 841 |
| | agaaggacta tcaactcaat gtgaatgtaa tattaaagtg aaagatgtca acgataactt | 901 |
| | cccaatgttt agagactctc agtattcagc acgtattgaa gaaaatattt taagttctga | 961 |
| | attacttcga tttcaagtaa cagatttgga tgaagagtac acagataatt ggcttgcagt | 1021 |
| | atatttcttt acctctggga atgaaggaaa ttggtttgaa atacaaactg atcctagaac | 1081 |
| | taatgaaggc atcctgaaag tggtgaaggc tctagattat gaacaactac aaagcgtgaa | 1141 |
| | acttagtatt gctgtcaaaa acaaagctga atttcaccaa tcagttatct ctcgataccg | 1201 |
| | agttcagtca accccagtca caattcaggt aataaatgta agagaaggaa ttgcattccg | 1261 |
| | tcctgcttcc aagacattta ctgtgcaaaa aggcataagt agcaaaaaat tggtggatta | 1321 |
| | tatcctggga acatatcaag ccatcgatga ggacactaac aaagctgcct caaatgtcaa | 1381 |
| | atatgtcatg ggacgtaacg atggtggata cctaatgatt gattcaaaaa ctgctgaaat | 1441 |
| | caaatttgtc aaaaatatga accgagattc tactttcata gttaacaaaa caatcacagc | 1501 |
| | tgaggttctg gccatagatg aatacacggg taaaacttct acaggcacgg tatatgttag | 1561 |
| | agtacccgat ttcaatgaca attgtccaac agctgtcctc gaaaaagatg cagtttgcag | 1621 |
| | ttcttcacct tccgtggttg tctccgctag aacactgaat aatagataca ctggccccta | 1681 |
| | tacatttgca ctggaagatc aacctgtaaa gttgcctgcc gtatggagta tcacaaccct | 1741 |
| | caatgctacc tcggccctcc tcagagccca ggaacagata cctcctggag tataccacat | 1801 |
| | ctccctggta cttacagaca gtcagaacaa tcggtgtgag atgccacgca gcttgacact | 1861 |
| | ggaagtctgt cagtgtgaca acaggggcat ctgtggaact tcttacccaa ccacaagccc | 1921 |
| | tgggaccagg tatggcaggc cgcactcagg gaggctgggg cctgccgcca tcggcctgct | 1981 |
| | gctccttggt ctcctgctgc tgctgttggc cccccttctg ctgttgacct gtgactgtgg | 2041 |
| | ggcaggttct actgggggag tgacaggtgg ttttatccca gttcctgatg gctcagaagg | 2101 |
| | aacaattcat cagtggggaa ttgaaggagc ccatcctgaa gacaaggaaa tcacaaatat | 2161 |
| | ttgtgtgcct cctgtaacag ccaatggagc cgatttcatg gaaagttctg aagtttgtac | 2221 |
| | aaatacgtat gccagaggca cagcggtgga aggcacttca ggaatggaaa tgaccactaa | 2281 |
| | gcttggagca gccactgaat ctggaggtgc tgcaggcttt gcaacaggga cagtgtcagg | 2341 |
| | agctgcttca ggattcggag cagccactgg agttggcatc tgttcctcag ggcagtctgg | 2401 |
| | aaccatgaga acaaggcatt ccactggagg aaccaataag gactacgctg atggggcgat | 2461 |
| | aagcatgaat tttctggact cctacttttc tcagaaagca tttgcctgtg cggaggaaga | 2521 |
| | cgatggccag gaagcaaatg actgcttgtt gatctatgat aatgaaggcg cagatgccac | 2581 |
| | tggttctcct gtgggctccg tgggttgttg cagttttatt gctgatgacc tggatgacag | 2641 |
| | cttcttggac tcacttggac ccaaatttaa aaaacttgca gagataagcc ttggtgttga | 2701 |
| | tggtgaaggc aaagaagttc agccaccctc taaagacagc ggttatggga ttgaatcctg | 2761 |
| | tggccatccc atagaagtcc agcagacagg atttgttaag tgccagactt tgtcaggaag | 2821 |
| | tcaaggagct tctgctttgt ccacctctgg gtctgtccag ccagctgttt ccatccctga | 2881 |
| | ccctctgcag catggtaact atttagtaac ggagacttac tcggcttctg gttccctcgt | 2941 |
| | gcaaccttcc actgcaggct ttgatccact tctcacacaa aatgtgatag tgacagaaag | 3001 |
| | ggtgatctgt cccatttcca gtgttcctgg caacctagct ggcccaacgc agctacgagg | 3061 |
| | gtcacatact atgctctgta cagaggatcc ttgctcccgt ctaatatgac cagaatgagc | 3121 |
| | tggaatacca cactgaccaa atctggatct ttggactaaa gtattcaaaa tagcatagca | 3181 |
| | aagctcactg tattgggcta ataatttggc acttattagc ttctctcata aactgatcac | 3241 |
| | gattataaat taaatgtttg ggttcatacc ccaaaagcaa tatgttgtca ctcctaattc | 3301 |
| | tcaagtacta ttcaaattgt agtaaatctt aaagtttttc aaaaccctaa aatcatattc | 3361 |
| | gccaggaaat tttcctaaac attcttaagc ttctattttt cccctgccaa aggaaggtgt | 3421 |
| | ttatcatttt aaaatgcaat gtgatttagt ggattaagca ggagcgctgg ttattgtctc | 3481 |
| | cattgccttt tcttatatca ttgataatga tgtaagaatc acaaggggcc gggcgcggtg | 3541 |
| | gctcacgcct gtaatcccag cactttggga ggccgaggca ggtggatcat gaggtcagga | 3601 |
| | gatcgagacc atcctggcta acaaggtgaa accccgtctc tactaaaaat acaaaaaatt | 3661 |
| | agccgggcgc agtggcgggc gcctgtagtc ccagctactc gggaggctga ggcaggagaa | 3721 |
| | tggcatgaac ccgggaagcg gagcttgcag tgagccgaga ttgcgccact gcagtccgca | 3781 |
| | gtccggcctg ggcgacagag cgagactccg tctcaaaaaa aaaaaaaaaa aaagaatcac | 3841 |
| | aaggtatttg ctaaagcatt ttgagctgct tggaaaaagg gaagtagttg cagtagagtt | 3901 |
| | tcttccatct tcttggtgct gggaagccat atatgtgtct tttactcaag ctaaggggta | 3961 |
| | taagcttatg tgttgaattt gctacatcta tatttcacat attctcacaa taagagaatt | 4021 |
| | ttgaaataga aatatcatag aacatttaag aaagtttagt ataaataata ttttgtgtgt | 4081 |
| | tttaatccct ttgaagggat ctatccaaag aaaatatttt acactgagct ccttcctaca | 4141 |
| | cgtctcagta acagatcctg tgttagtctt tgaaaatagc tcatttttta aatgtcagtg | 4201 |
| | agtagatgta gcatacatat gatgtataat gacgtgtatt atgttaacaa tgtctgcaga | 4261 |
| | ttttgtagga atacaaaaca tggccttttt tataagcaaa acgggccaat gactagaata | 4321 |
| | acacataggg caatctgtga atatgtatta taagcagcat tccagaaaag tagttggtga | 4381 |
| | aataattttc aagtcaaaaa gggatatgga aagggaatta tgagtaacct ctatttttta | 4441 |
| | agccttgctt ttaaattaaa cagctacagc catttaagcc ttgaggataa taaagcttga | 4501 |
| | gagtaataat gttaggttag caaaggttta gatgtatcac ttcatgcatg ctaccatgat | 4561 |
| | agtaatgcag ctcttcgagt catttctggt cattcaagat attcaccctt ttgcccatag | 4621 |
| | aaagcaccct acctcacctg cttactgaca ttgtcttagc tgatcacaag atcattatca | 4681 |
| | gcctccatta ttccttactg tatataaaat acagagtttt atattttcct ttcttcgttt | 4741 |
| | ttcaccatat tcaaaaccta aatttgtttt tgcagatgga atgcaaagta atcaagtgtt | 4801 |
| | tgtgctttca cctagaaggg tgtggtcctg aaggaaagag gtcccctaaa tatcccccac | 4861 |
| | cctggtgctc ctccctctcc ctggtaccct gactaccagg aagtcaggtg ctagagcagc | 4921 |
| | tggagaagtg caggcagcct gtgcttccac agatgggggt gctgctgcaa caaggctttc | 4981 |
| | aatgtgccca tcttaggtgg gagaagctag atcctgtgca gcagcctggt aagtcctgag | 5041 |
| | gaggttccat tgctcttcct gctgctgtcc tttgcttctc aacggtggct cgctctacag | 5101 |
| | tctagagcac atgcagctaa cttgtgcctc tgcttatgca tgagggttaa attaacaacc | 5161 |
| | ataaccttca tttgaagttc aaaggtgtat tcaggatcct caaagcattt taaccttgcc | 5221 |
| | gcttaaaacc caatttaccg tgaaatggga attttgctgc attgttaaac tgtagtggaa | 5281 |
| | accatgctat agtaataaag gttatataag agagaaattg aaattaaatg tgtttttaaa | 5341 |
| | tttcaaaaaa aaatcaatct ttaggatgac ttaaaaattg atttgccatg taaaatgtat | 5401 |
| | ctgcattttt tacacaaaac ttgttttaag cataaaattt taaaactgta ctacttgatg | 5461 |
| | tattatacat tttgaaccat atgtattaaa ccataaacag tataatgttg ttataataaa | 5521 |
| | acaggcaata aatttataaa taaaagctga aaaaaaaaaa | |

Homo sapiens thymidine phosphorylase (TYMP), transcript variant 3, mRNA
NCBI Reference Sequence: NM_001113756.1 (SEQ ID. NO. 42)

| | | |
|---|---|---|
| 1 | cgaggggcgg acaccggaga gacacgggaa aggggtcggg acaggagcac gtggctcaga | |
| 61 | caccgacgcc gggaggccgc agaccccgga cgtgtcaggc atccccgcag gcccggagcg | 121 |
| | atggcagcct tgatgacccc gggaaccggg gccccacccg cgcctggtga cttctccggg | 181 |
| | gaagggagcc agggacttcc cgacccttcg ccagagccca agcagctccc ggagctgatc | 241 |
| | cgcatgaagc gagacggagg ccgcctgagc gaagcggaca tcaggggctt cgtggccgct | 301 |
| | gtggtgaatg ggagcgcgca gggcgcacag atcggggcca tgctgatggc catccgactt | 361 |
| | cggggcatgg atctggagga gacctcggtg ctgacccagg ccctggctca gtcgggacag | 421 |
| | cagctggagt ggccagaggc ctggcgccag cagcttgtgg acaagcattc cacagggggt | 481 |
| | gtgggtgaca aggtcagcct ggtcctcgca cctgccctgg cggcatgtgg ctgcaaggtg | 541 |
| | ccaatgatca gcggacgtgg tctggggcac acaggaggca ccttggataa gctggagtct | 601 |
| | attcctggat tcaatgtcat ccagagccca gagcagatgc aagtgctgct ggaccaggcg | 661 |
| | ggctgctgta tcgtgggtca gagtgagcag ctggttcctg cggacggaat cctatatgca | 721 |
| | gccagagatg tgacagccac cgtggacagc ctgccactca tcacagcctc cattctcagt | 781 |
| | aagaaactcg tggaggggct gtccgctctg gtggtggacg ttaagttcgg aggggccgcc | 841 |
| | gtcttcccca accaggagca ggcccgggag ctggcaaaga cgctggttgg cgtgggagcc | 901 |
| | agcctagggc ttcgggtcgc ggcagcgctg accgccatgg acaagcccct gggtcgctgc | 961 |
| | gtgggccacg ccctggaggt ggaggaggcg ctgctctgca tggacggcgc aggcccgcca | 1021 |
| | gacttaaggg acctggtcac cacgctcggg ggcgccctgc tctggctcag cggacacgcg | 1081 |
| | gggactcagg cccagggcgc tgcccgggtg gccgcggcgc tggacgacgg ctcggccctt | 1141 |
| | ggccgcttcg agcggatgct ggcggcgcag ggcgtggatc ccggtctggc ccgagccctg | 1201 |
| | tgctcgggaa gtcccgcaga acgccggcag ctgctgcctc gcgcccggga gcaggaggag | 1261 |
| | ctgctggcgc ccgcagatgg caccgtggag ctggtccggg cgctgccgct ggcgctggtg | 1321 |
| | ctgcacgagc tcggggccgg gcgcagccgc gctggggagc cgctccgcct gggggtgggc | 1381 |
| | gcagagctgc tggtcgacgt gggtcagagg ctgcgccgtg ggaccccctg gctccgcgtg | 1441 |
| | caccgggacg gccccgcgct cagcggcccg cagagccgcg ccctgcagga ggcgctcgta | 1501 |
| | ctctccgacc gcgcgccatt cgccgccccc tcgcccttcg cagagctcgt tctgccgccg | 1561 |
| | cagcaataaa gctcctttgc cgcgaaa | |

Homo sapiens keratin 6A (KRT6A), mRNA
NCBI Reference Sequence: NM_005554.3 (SEQ ID. NO. 43)

| | | |
|---|---|---|
| 1 | atatttcata cctttctaga aactgggtgt gatctcactg ttggtaaagc ccagcccttc | |
| 61 | ccaacctgca agctcacctt ccaggactgg gcccagccca tgctctccat atataagctg | 121 |
| | ctgccccgag cctgattcct agtcctgctt ctcttccctc tctcctccag cctctcacac | 181 |
| | tctcctcagc tctctcatct cctggaacca tggccagcac atccaccacc atcaggagcc | 241 |
| | acagcagcag ccgccggggt ttcagtgcca actcagccag gctccctggg gtcagccgct | 301 |
| | ctggcttcag cagcgtctcc gtgtcccgct ccaggggcag tggtggcctg ggtggtgcat | 361 |
| | gtggaggagc tggctttggc agccgcagtc tgtatggcct ggggggctcc aagaggatct | 421 |
| | ccattggagg gggcagctgt gccatcagtg gcggctatgg cagcagagcc ggaggcagct | 481 |
| | atggctttgg tggcgccggg agtggatttg gtttcggtgg tggagccggc attggctttg | 541 |
| | gtctgggtgg tggagccggc cttgctggtg gctttggggg ccctggcttc cctgtgtgcc | 601 |
| | cccctggagg catccaagag gtcaccgtca accagagtct cctgactccc ctcaacctgc | 661 |
| | aaatcgatcc caccatccag cgggtgcggg ctgaggagcg tgaacagatc aagaccctca | 721 |
| | acaacaagtt tgcctccttc atcgacaagg tgcggttcct ggagcagcag aacaaggttc | 781 |
| | tggaaacaaa gtggaccctg ctgcaggagc agggcaccaa gactgtgagg cagaacctgg | 841 |
| | agccgttgtt cgagcagtac atcaacaacc tcaggaggca gctggacagc attgtcgggg | 901 |
| | aacggggccg cctggactca gagctcagag gcatgcagga cctggtggag gacttcaaga | 961 |
| | acaaatatga ggatgaaatc aacaagcgca cagcagcaga gaatgaattt gtgactctga | 1021 |
| | agaaggatgt ggatgctgcc tacatgaaca aggttgaact gcaagccaag gcagacactc | 1081 |
| | tcacagacga gatcaacttc ctgagagcct tgtatgatgc agagctgtcc cagatgcaga | 1141 |
| | cccacatctc agacacatct gtggtgctgt ccatggacaa caaccgcaac ctggacctgg | 1201 |
| | acagcatcat cgctgaggtc aaggcccaat atgaggagat tgctcagaga agccgggctg | 1261 |
| | aggctgagtc ctggtaccag accaagtacg aggagctgca ggtcacagca ggcagacatg | 1321 |
| | gggacgacct gcgcaacacc aagcaggaga ttgctgagat caaccgcatg atccagaggc | 1381 |
| | tgagatctga gatcgaccac gtcaagaagc agtgcgccaa cctgcaggcc gccattgctg | 1441 |
| | atgctgagca gcgtggggag atggccctca aggatgccaa gaacaagctg gaagggctgg | 1501 |
| | aggatgccct gcagaaggcc aagcaggacc tggcccggct gctgaaggag taccaggagc | 1561 |
| | tgatgaatgt caagctggcc ctggacgtgg agatcgccac ctaccgcaag ctgctggagg | 1621 |
| | gtgaggagtg caggctgaat ggcgaaggcg ttggacaagt caacatctct gtggtgcagt | 1681 |
| | ccaccgtctc cagtggctat ggcggtgcca gtggtgtcgg cagtggctta ggcctgggtg | 1741 |
| | gaggaagcag ctactcctat ggcagtggtc ttggcgttgg aggtggcttc agttccagca | 1801 |
| | gtggcagagc cattgggggt ggcctcagct ctgttggagg cggcagttcc accatcaagt | 1861 |
| | acaccaccac ctcctcctcc agcaggaaga gctataagca ctaaagtgcg tctgctagct | 1921 |
| | ctcggtccca cagtcctcag gcccctctct ggctgcagag ccctctcctc aggttgcctt | 1981 |
| | tcctctcctg gcctccagtc tcccctgctg tcccaggtag agctgggtat ggatgcttag | 2041 |
| | tgccctcact tcttctctct ctctctatac catctgagca cccattgctc accatcagat | 2101 |
| | caacctctga ttttacatca tgatgtaatc accactggag cttcactgtt actaaattat | 2161 |
| | taatttcttg cctccagtgt tctatctctg aggctgagca ttataagaaa atgacctctg | 2221 |
| | ctccttttca ttgcagaaaa ttgccagggg cttatttcag aacaacttcc acttactttc | 2281 |
| | cactggctct caaactctct aacttataag tgttgtgaac ccccacccag gcagtatcca | 2341 |
| | tgaaagcaca agtgactagt cctatgatgt acaaagcctg tatctctgtg atgatttctg | 2401 |
| | tgctcttcgc tgtttgcaat tgctaaataa agcagattta taataca | |

Homo sapiens keratin 6B (KRT6B), mRNA
NCBI Reference Sequence: NM_005555.3 (SEQ ID. NO. 44)

| | | |
|---|---|---|
| 1 | cgcctccagc ctctcacact ctcctaagcc ctctcatctc ctggaaccat ggccagcaca | |
| 61 | tccaccacca tcaggagcca cagcagcagc cgccggggtt tcagtgccaa ctcagccagg | 121 |
| | ctccctgggg tcagccgctc tggcttcagc agcatctccg tgtcccgctc caggggcagt | 181 |
| | ggtggcctgg gtggcgcatg tggaggagct ggctttggca gccgcagtct gtatggcctg | 241 |
| | gggggctcca agaggatctc cattggaggg ggcagctgtg ccatcagtgg cggctatggc | 301 |
| | agcagagccg gaggcagcta tggctttggt ggcgccggga gtggatttgg tttcggtggt | 361 |
| | ggagccggca ttggctttgg tctgggtggt ggagccggcc ttgctggtgg ctttgggggc | 421 |
| | cctggcttcc ctgtgtgccc ccctggaggc atccaagagg tcactgtcaa ccagagtctc | 481 |
| | ctgactcccc tcaacctgca aattgacccc gccatccagc gggtgcgggc cgaggagcgt | 541 |
| | gagcagatca agaccctcaa caacaagttt gcctccttca tcgacaaggt gcggttccta | 601 |
| | gagcagcaga acaaggttct ggacaccaag tggaccctgc tgcaggagca gggcaccaag | 661 |
| | actgtgaggc agaacctgga gccgttgttc gagcagtaca tcaacaacct caggaggcag | 721 |
| | ctggacaaca tcgtggggga acggggtcgt ctggactcgg agctgagaaa catgcaggac | 781 |
| | ctggtggagg acctcaagaa caaatatgag gatgaaatca acaagcgcac agcagcagag | 841 |
| | aatgaatttg tgactctgaa gaaggatgtg gatgctgcct acatgaacaa ggttgaactg | 901 |
| | caagccaagg cagacactct tacagatgag atcaacttcc tgagagcctt gtatgatgca | 961 |
| | gagctgtccc agatgcagac ccacatctca gacacatccg tggtgatatc catggacaac | 1021 |
| | aaccgcaacc tggacctgga cagcatcatc gctgaggtca aggcccaata tgaggagatt | 1081 |
| | gctcagagga gcagggctga ggctgagtcc tggtaccaga caaagtacga ggagctgcag | 1141 |
| | atcacagcag gcagacatgg ggacgacctg cgcaacacca agcaggagat tgctgagatc | 1201 |
| | aaccgcatga tccagaggct gagatctgag atcgaccacg tcaagaagca gtgtgccaac | 1261 |
| | ctacaggccg ccattgctga tgctgagcag cgtggggaga tggccctcaa ggatgctaag | 1321 |
| | aacaagctgg aagggctgga ggatgccctg cagaaggcca agcaggacct ggcccggctg | 1381 |
| | ctgaaggagt accaggagct gatgaacgtc aagctggccc tggatgtgga gatcgccacc | 1441 |
| | taccgcaagc tgctggaggg cgaggagtgc aggctgaatg gcgaaggcgt tggacaagtc | 1501 |
| | aacatctctg tagtgcagtc caccgtctcc agtggctatg gcggtgccag cggtgtcggc | 1561 |
| | agtggcttag gcctgggtgg aggaagcagc tactcctatg gcagtggtct tggcgttgga | 1621 |
| | ggcggcttta gttccagcag cggcagagcc actgggggtg gcctcagctc tgttggaggc | 1681 |
| | ggcagttcca ccatcaagta caccaccacc tcctcctcca gcaggaagag ctacaagcac | 1741 |
| | tgaagtgctg ccgccagctc tcagtcccac agctctcagg cccctctctg gcagcagagc | 1801 |
| | cctctcctca ggttgcttgt cctcccctgg cctccagtct cccctgccct cccgggtaga | 1861 |
| | gctgggatgc cctcactttt cttctcatca atacctgttc cactgagctc ctgttgctta | 1921 |
| | ccatcaagtc aacagttatc agcactcaga catgcgaatg tcctttttag ttcccgtatt | 1981 |
| | attacaggta tctgagtctg ccataattct gagaagaaaa tgacctatat ccccataaga | 2041 |
| | actgaaactc agtctaggtc cagctgcaga tgaggagtcc tctctttaat tgctaaccat | 2101 |
| | cctgcccatt atagctacac tcaggagttc tcatctgaca agtcagttgt cctgatcttc | 2161 |
| | tcttgcagtg tccctgaatg gcaagtgatg taccttctga tgcagtctgc attcctgcac | 2221 |
| | tgctttctct gctctctttg ccttcttttg ttctgttgaa taaagcatat tgagaatgtg | 2281 |
| | aaaaaaaaaa aaaaaaaaaa aaaaaaaaaa aaaaaaaaaa aaaaaaaaaa | |

Homo sapiens major histocompatibility complex, class II, DR beta 1 (HLA-
DRB1), mRNA
NCBI Reference Sequence: NM_002124.1 (SEQ ID. NO. 45)

| | | |
|---|---|---|
| 1 | tagttctccc tgagtgagac ttgcctgctt ctctggcccc tggtcctgtc ctgttctcca | |
| 61 | gcatggtgtg tctgaagctc cctggaggct cctgcatgac agcgctgaca gtgacactga | 121 |
| | tggtgctgag ctccccactg gctttggctg gggacacccg accacgtttc ttgtggcagc | 181 |
| | ttaagtttga atgtcatttc ttcaatggga cggagcgggt gcggttgctg gaaagatgca | 241 |
| | tctataacca agaggagtcc gtgcgcttcg acagcgacgt gggggagtac cgggcggtga | 301 |
| | cggagctggg gcggcctgat gccgagtact ggaacagcca gaaggacctc ctggagcaga | 361 |
| | ggcgggccgc ggtggacacc tactgcagac acaactacgg ggttggtgag agcttcacag | 421 |
| | tgcagcggcg agttgagcct aaggtgactg tgtatccttc aaagacccag cccctgcagc | 481 |
| | accacaacct cctggtctgc tctgtgagtg gtttctatcc aggcagcatt gaagtcaggt | 541 |
| | ggttccggaa cggccaggaa gagaaggctg gggtggtgtc cacaggcctg atccagaatg | 601 |
| | gagattggac cttccagacc ctggtgatgc tggaaacagt tcctcggagt ggagaggttt | 661 |
| | acacctgcca agtggagcac ccaagtgtga cgagccctct cacagtggaa tggagagcac | 721 |
| | ggtctgaatc tgcacagagc aagatgctga gtggagtcgg gggcttcgtg ctgggcctgc | 781 |
| | tcttccttgg ggccgggctg ttcatctact tcaggaatca gaaaggacac tctggacttc | 841 |
| | agccaacagg attcctgagc tgaaatgcag atgaccacat tcaaggaaga accttctgtc | 901 |
| | ccagctttgc agaatgaaaa gctttcctgc ttggcagtta ttcttccaca agagagggct | 961 |
| | ttctcaggac ctggttgcta ctggttcggc aactgcagaa aatgtcctcc cttgtggctt | 1021 |
| | cctcagctcc tgcccttggc ctgaagtccc agcattgatg acagcgcctc atcttcaact | 1081 |
| | tttgtgctcc cctttgccta aaccgtatgg cctcccgtgc atctgtactc accctgtacg | 1141 |
| | acaaacacat tacattatta aatgtttctc aaagatggag | |

Homo sapiens lipocalin 2 (LCN2), mRNA
NCBI Reference Sequence: NM_005564.3 (SEQ ID. NO. 46)

| | | |
|---|---|---|
| 1 | actcgccacc tcctcttcca cccctgccag gcccagcagc caccacagcg cctgcttcct | |
| 61 | cggccctgaa atcatgcccc taggtctcct gtggctgggc ctagccctgt tgggggctct | 121 |
| | gcatgcccag gcccaggact ccacctcaga cctgatccca gccccacctc tgagcaaggt | 181 |
| | ccctctgcag cagaacttcc aggacaacca attccagggg aagtggtatg tggtaggcct | 241 |
| | ggcagggaat gcaattctca gagaagacaa agacccgcaa aagatgtatg ccaccatcta | 301 |
| | tgagctgaaa gaagacaaga gctacaatgt cacctccgtc ctgtttagga aaaagaagtg | 361 |
| | tgactactgg atcaggactt ttgttccagg ttgccagccc ggcgagttca cgctgggcaa | 421 |
| | cattaagagt taccctggat taacgagtta cctcgtccga gtggtgagca ccaactacaa | 481 |
| | ccagcatgct atggtgttct tcaagaaagt ttctcaaaac agggagtact tcaagatcac | 541 |
| | cctctacggg agaaccaagg agctgacttc ggaactaaag gagaacttca tccgcttctc | 601 |
| | caaatctctg ggcctccctg aaaaccacat cgtcttccct gtcccaatcg accagtgtat | 661 |
| | cgacggctga gtgcacaggt gccgccagct gccgcaccag cccgaacacc attgagggag | 721 |
| | ctgggagacc ctccccacag tgccacccat gcagctgctc cccaggccac cccgctgatg | 781 |
| | gagccccacc ttgtctgcta aataaacatg tgccctcagg ccaaaaaaaa aaaaaa | |

Homo sapiens keratin 4 (KRT4), mRNA
NCBI Reference Sequence: NM_002272.2 (SEQ ID. NO. 47)

| | | |
|---|---|---|
| 1 | gacttgctcc ggtttgcaga gctaggaggt ggcaggctgt gcgctcaaac tcaggctgtc | |
| 61 | taactccaca ttctgtgggg tgagaggatg ggtgatgggg tgtcttttct ggaggaggga | 121 |
| | ggtgctgtga gcctagcgag atggaggtac agtgggtgtg ggcctggagc gctgggccca | 181 |
| | ggcaggggct tctgattagg aagccctggg gcaccagttc aggttctccc agagagtagt | 241 |
| | gtgatgggat ccagtaacct gtgccctcca gatgacttct gtaggtgtgt ttagtgacat | 301 |
| | gctcaacggg tgcgggaagg atgggcttgt gccaagggcc aagcccagag atgtttcaga | 361 |
| | tttttccctt tatgcccctg caaccaagcc ctgctgctcc aggacatata agagacgaag | 421 |
| | gctgagggct ccagcactca ccggcctggg ccctgtcact tctctgatag ctcccagctc | 481 |
| | gctctctgca gccatgattg ccagacagca gtgtgtccga ggcgggcccc ggggcttcag | 541 |
| | ctgtggctcg gccattgtag gcggtggcaa gagaggtgcc ttcagctcag tctccatgtc | 601 |
| | tggaggtgct ggccgatgct cttctggggg atttggcagc agaagcctct acaacctcag | 661 |
| | ggggaacaaa agcatctcca tgagtgtggc tgggtcacga caaggtgcct gctttggggg | 721 |
| | tgctggaggc tttggcactg gtggctttgg tggtggattt gggggctcct tcagtggtaa | 781 |
| | gggtggccct ggcttccccg tctgccccgc tgggggaatt caggaggtca ccatcaacca | 841 |
| | gagcttgctc acccccctcc acgtggagat tgaccctgag atccagaaag tccggacgga | 901 |
| | agagcgcgaa cagatcaagc tcctcaacaa caagtttgcc tccttcatcg acaaggtgca | 961 |
| | gttcttagag caacagaata aggtcctgga gaccaaatgg aacctgctcc agcagcagac | 1021 |
| | gaccaccacc tccagcaaaa accttgagcc cctctttgag acctacctca gtgtcctgag | 1081 |
| | gaagcagcta gataccttgg gcaatgacaa agggcgcctg cagtctgagc tgaagaccat | 1141 |
| | gcaggacagc gtggaggact tcaagactaa gtatgaagag gagatcaaca aacgcacagc | 1201 |
| | agccgagaat gactttgtgg tcctaaagaa ggacgtggat gctgcctacc tgaacaaggt | 1261 |
| | ggagttggag gccaaggtgg acagtcttaa tgacgagatc aacttcctga aggtcctcta | 1321 |
| | tgatgcggag ctgtcccaga tgcagaccca tgtcagcgac acgtccgtgg tcctttccat | 1381 |
| | ggacaacaac cgcaacctgg acctggacag cattattgcc gaggtccgtg cccagtacga | 1441 |
| | ggagattgcc cagaggagca aggctgaggc tgaagccctg taccagacca aggtccagca | 1501 |
| | gctccagatc tcggttgacc aacatggtga caacctgaag aacaccaaga gtgaaattgc | 1561 |
| | agagctcaac aggatgatcc agaggctgcg ggcagagatc gagaacatca agaagcagtg | 1621 |
| | ccagactctt caggtatccg tggctgatgc agagcagcga ggtgagaatg cccttaaaga | 1681 |
| | tgcccacagc aagcgcgtag agctggaggc tgccctgcag caggccaagg aggagctggc | 1741 |
| | acgaatgctg cgtgagtacc aggagctcat gagtgtgaag ctggccttgg acatcgagat | 1801 |
| | cgccacctac cgcaaactgc tggagggcga ggagtacaga atgtctggag aatgccagag | 1861 |
| | tgccgtgagc atctctgtgg tcagcggtag caccagcact ggaggcatca gcggaggatt | 1921 |
| | aggaagtggc tccgggtttg gcctgagtag tggctttggc tccggctctg gaagtggctt | 1981 |
| | tgggtttggt ggcagtgtct ctggcagttc cagcagcaag atcatctcta ccaccaccct | 2041 |
| | gaacaagaga cgatagagga gacgaggtcc ctgcagctca ctgtgtccag ctgggcccag | 2101 |
| | cactggtgtc tctgtgcttc cttcacttca cctccatcct ctgtctctgg ggctcatctt | 2161 |
| | actagtatcc cctccactat cccatgggct ctctctgccc caggatgatc ttctgtgctg | 2221 |
| | ggacagggac tctgcctctt ggagtttggt agctacttct tgatttgggc ctggtgaccc | 2281 |
| | acctggaatg ggaaggatgt cagctgacct ctcacctccc atggacagag aagaaaatga | 2341 |
| | ccaggagtgt catctccaga attattgggg tcacatatgt cccttcccag tccaatgcca | 2401 |
| | tctcccacta gatcctgtat tatccatcta catcagaacc aaactacttc tccaacaccc | 2461 |
| | ggcagcactt ggccctgcaa gcttaggatg agaaccactt agtgtcccat tctactcctc | 2521 |
| | tcattccctc ttatccatct gcaggtgaat cttcaataaa atgcttttgt cattca | |

Homo sapiens interferon, gamma-inducible protein 30 (IFI30), mRNA
NCBI Reference Sequence: NM_006332.3 (SEQ ID. NO. 48)

| | | |
|---|---|---|
| 1 | ggaccgccgc ctggttaaag gcgcttattt cccaggcagc cgctgcagtc gccacacctt | |
| 61 | tgcccctgct gcgatgaccc tgtcgccact tctgctgttc ctgccaccgc tgctgctgct | 121 |
| | gctggacgtc cccacggcgg cggtgcaggc gtcccctctg caagcgttag acttctttgg | 181 |
| | gaatgggcca ccagttaact acaagacagg caatctatac ctgcgggggc ccctgaagaa | 241 |
| | gtccaatgca ccgcttgtca atgtgaccct ctactatgaa gcactgtgcg gtggctgccg | 301 |
| | agccttcctg atccgggagc tcttcccaac atggctgttg gtcatggaga tcctcaatgt | 361 |
| | cacgctggtg ccctacggaa acgcacagga acaaaatgtc agtggcaggt gggagttcaa | 421 |
| | gtgccagcat ggagaagagg agtgcaaatt caacaaggtg gaggcctgcg tgttggatga | 481 |
| | acttgacatg gagctagcct tcctgaccat tgtctgcatg gaagagtttg aggacatgga | 541 |
| | gagaagtctg ccactatgcc tgcagctcta cgccccaggg ctgtcgccag acactatcat | 601 |
| | ggagtgtgca atgggggacc gcggcatgca gctcatgcac gccaacgccc agcggacaga | 661 |
| | tgctctccag ccaccacacg agtatgtgcc ctgggtcacc gtcaatggga aacccttgga | 721 |
| | agatcagacc cagctcctta cccttgtctg ccagttgtac cagggcaaga agccggatgt | 781 |
| | ctgcccttcc tcaaccagct ccctcaggag tgtttgcttc aagtgatggc cggtgagctg | 841 |
| | cggagagctc atggaaggcg agtgggaacc cggctgcctg cctttttttc tgatccagac | 901 |
| | cctcggcacc tgctacttac caactggaaa attttatgca tcccatgaag cccagataca | 961 |
| | caaaattcca ccccatgatc aagaatcctg ctccactaag aatggtgcta aagtaaaact | 1021 |
| | agtttaataa gcaaaaaaaa aaaaaaaaaa | |

PREDICTED: Homo sapiens hypothetical protein LOC100134370 (LOC100134370),
mRNA
NCBI Reference Sequence: XM_001713687.1 (SEQ ID. NO. 49)

| | | |
|---|---|---|
| 1 | gttccatcct ctgccatcta ctccactgtt cagacacctc ctaacctccg tcatgacctg | |
| 61 | tggcttcaac tccataggct gtgggttccg ccctggaaac ttcagctgtg tctctgcctg | 121 |
| | cgggccccgg ccaagccgct gctgcatcac cgccgccccc taccgcggca tctcctgcta | 181 |
| | ccgcggcctc accgggggct ttggcagcca cagcgtgtgc gggggcttcc gcgccggctc | 241 |
| | ctgcggacgc agcttcggct accgctccgg gggcgtgtgc ggacccagcc ccccatgcat | 301 |
| | caccaccgtg tcggtcaacg agagcctcct cacgcccctc aacctggaga tagaccccaa | 361 |
| | cgcgcagtgc gtgaagcagg aggagaagga gcagatcaag tccctcaaca gcagattcgc | 421 |
| | ggccttcatc gacaaggtgc gcttcctgga gcagcagaac aagctgctgg agacaaagct | 481 |
| | gcagttctac caaaaccgcg agtgctgcca gagtaacctg gagcccctgt ttgctggcta | 541 |
| | catcgagact ctgcggcggg aggccgagtg cgtggaggct gacagtggga ggctggcctc | 601 |
| | agagctcaat cacgtgcagg aggtgctgga gggctacaag aagaagtatg aagaagaagt | 661 |
| | agcacttcga gccacagcag agaacgagtt tgtggctcta aagaaggatg tggactgcgc | 721 |
| | ctacctccgc aagtcagacc tggaggccaa cgtggaggcc ctgatccagg agattgactt | 781 |
| | cctgaggcgg ctgtacgagg aggagatccg cattctccaa tcccacatct cagacacctc | 841 |
| | cgtggttgtc aagctggaca acagccggga cctgaacatg gactgcatgg ttgctgagat | 901 |
| | caaggcacag tatgatgaca ttgccacccg tagccgggct gaggccgagt cctggtatcg | 961 |
| | cagcaagtgt gaggagatga aggccacagt gatcaggcac ggggagaccc tgcgccgcac | 1021 |
| | caaggaggag atcaatgagc tgaaccgcat gatccagagg ctgacagccg aggtggagaa | 1081 |
| | tgccaagtgc cagaactcca agctggaggc cgcggtggcc cagtctgagc agcagggtga | 1141 |
| | ggcggccctc agtgatgccc gctgcaagct ggccgagctg gagggcgccc tgcagaaggc | 1201 |
| | caagcaagac atggcctgcc tgatcaggga gtaccaggag gtgatgaact ccaagctagg | 1261 |
| | cctggatatc gagatcgcca cctacaggcg cctgctggag ggcgaggagc ataggctgtg | 1321 |
| | tgaaggtgtt gaagctgtga atgtctgtgt cagcagctcc cggggtgggg ttgtgtgcgg | 1381 |
| | ggacctctgc gtgtcgggct cccggccggt gacgggcagc gtctgcagtg ccccctgcaa | 1441 |
| | cgggaacctg gtggtgagca ctggtttgtg caagccctgt ggccagctga acaccacctg | 1501 |
| | tggagggggc tcctgcggcc aggggaggta ttaagtggcc caaaagagag ccaggggagc | 1561 |
| | cccttctgcc tgccagacgt gccactgccc caccaccagc tgaaaacagc agcacatcgc | 1621 |
| | tggcttttcc ccttgtgttc tgagaataca ccatcggctc attcccacca gcggctcctc | 1681 |
| | cccacctttc atcccactgg aaaggggtct gtggctgggg aatagaccca ttccttcccc | 1741 |
| | tgtctcagcc ttcagcccct cccggggaga agggccttgc ttccctggaa gaagcactgt | 1801 |
| | gagactgttc cccctgcctc tctggcctct tgtctcccct tttccaataa acttggggac | 1861 |
| | ctgc | |

Homo sapiens ring finger protein 213 (RNF213), transcript variant 2, mRNA
NCBI Reference Sequence: NM_020954.2 (SEQ ID. NO. 50)

| | | |
|---|---|---|
| 1 | cagcgcgcgg caggcggcga gctcgggggc cgcagaaaat gaaactgaag ccgtggtcac | |
| 61 | gtgacaggac atgtagtata tagcaggctg ccagcgactc ctgctcttgc ttctggatct | 121 |
| | gcagggcagt cccagcagga cccatggagt gtccttcgtg ccagcatgtc tccaaggagg | 181 |
| | aaacccccaa gttctgcagc cagtgcggag agaggctgcc tcctgcagcc cccatagcag | 241 |
| | attctgagaa caataactcc acaatggcgt cggcctcgga gggtgaaatg gagtgtgggc | 301 |
| | aggagctgaa ggaggaaggg ggcccgtgct tgttcccggg ctcagacagt tggcaagaaa | 361 |
| | accccgagga gccctgttcc aaagcctcct ggaccgtcca agaaagcaaa aagaagaaaa | 421 |
| | ggaagaagaa aaagaagggg aacaagtccg cttcctcaga gctggcttcc ttgccccttt | 481 |
| | ctcctgccag cccctgtcac ctgactttgc tttcaaaccc gtggcctcag gacacagccc | 541 |
| | tgccccacag ccaagcccag cagagtggcc ccactggcca gccgagccag cccccaggca | 601 |
| | cagccaccac gccactggag ggtgacggcc tctccgcgcc caccgaggtt ggcgacagcc | 661 |
| | ccctgcaggc ccaggctttg ggagaggcag gagtggccac aggaagtgag gctcagagca | 721 |
| | gcccgcaatt ccaggaccac acggaagggg aggaccagga cgcttccatc ccctctgggg | 781 |
| | gcagaggcct gtcccaggag gggaccggtc cccccacctc tgctggtgaa ggccattcta | 841 |
| | ggactgaaga tgctgcccag gagctcctgt tgcctgagtc aaaaggaggc agctctgagc | 901 |
| | ccgggacaga actgcagacc accgagcaac aggcaggggc ctcagcctct atggcagttg | 961 |
| | atgctgtagc tgagccagcc aatgcagtta aaggggccgg gaaggaaatg aaagagaaga | 1021 |
| | cccagagaat gaaacagcca ccagcaacca ctcctccttt caaaacacac tgccaggaag | 1081 |
| | ctgagaccaa gaccaaggac gagatggctg ctgctgaaga aaaagtcggt aagaatgaac | 1141 |
| | aaggggagcc tgaagacctc aagaagccag aggggaagaa cagaagtgca gctgctgtga | 1201 |
| | aaaacgagaa ggagcaaaaa aaccaggaag cagatgtcca ggaagtgaag gcaagcacgc | 1261 |
| | tgagcccggg tggaggagtc accgtgttct tccacgccat catctctctt catttcccat | 1321 |
| | tcaatcctga cctccataaa gtcttcatca gaggaggaga agaatttggg gagtcaaaat | 1381 |
| | gggacagcaa tatctgtgag ctgcactaca ccagagactt gggtcatgac cgcgttcttg | 1441 |
| | ttgaaggcat tgtctgcatt tccaagaagc acctagataa atacattcct tacaagtacg | 1501 |
| | tcatttataa tggggaatct tttgagtatg agttcattta caagcaccag cagaagaagg | 1561 |
| | gcgagtacgt caaccgctgt ctgttcataa aatcttcact tctgggctca ggagactggc | 1621 |
| | atcagtacta tgacatagtt tatatgaagc ctcatgggag actccagaaa gtcatgaacc | 1681 |
| | acatcacaga cgggccgagg aaggacctgg tgaaggggaa gcagattgcc gctgcgctca | 1741 |
| | tgctggacag caccttcagc atcctgcaga cctgggacac catcaacctg aacagcttct | 1801 |
| | tcacccagtt cgagcagttt tgctttgtcc tgcaacagcc tatgatttat gaaggacagg | 1861 |
| | cacagctgtg gaccgatttg cagtacaggg agaaagaggt gaagagatac ctgtggcaac | 1921 |
| | atctgaaaaa acacgtggta ccattgccgg acggaaaaag cacggacttt ttgcctgtgg | 1981 |
| | actgcccagt gaggagtaaa ctgaaaacag gcctgattgt cctttttgta gtggaaaaaa | 2041 |
| | ttgagctttt attagaaggc agcctggact ggttgtgtca cctcctaacc tcagatgcca | 2101 |
| | gctcaccaga tgagtttcac cgtgacctaa gccacatcct tgggatacct cagagctggc | 2161 |
| | ggctgtacct ggtgaacctg tgccaaagat gcatggacac aaggacgtac acctggctgg | 2221 |
| | gcgccctgcc tgtcctgcac tgctgtatgg agctggcccc gcggcacaag gatgcctgga | 2281 |
| | gacagcctga ggacacctgg gccgctctgg agggactctc cttctcaccg ttccgggaac | 2341 |
| | aaatgctaga tacgagttcc ctacttcagt ttatgagaga gaagcagcat ttgctgagca | 2401 |
| | tagacgagcc tctcttccgg tcctggttta gtctgctacc tctgagtcac ctggttatgt | 2461 |
| | atatggaaaa cttcattgag cacctgggtc gttttcctgc tcatatcctg gactgtcttt | 2521 |
| | cagggattta ctaccggctt ccgggacttg agcaagtctt gaatacgcag gatgttcagg | 2581 |
| | atgttcagaa cgttcagaac attttagaaa tgctgttgcg actcctggac acttaccggg | 2641 |
| | acaagattcc cgaggaggcc ttgtcaccat cctacctgac tgtgtgtctg aaactgcatg | 2701 |
| | aagccatctg cagcagcaca aagctactta agttttacga gctgccagcc ttatctgccg | 2761 |
| | agattgtctg cagaatgatt agacttctat ctctggtgga ttctgcagga cagagagatg | 2821 |
| | aaactggaaa taattcagtc caaacagtct tccaagggac ccttgctgct acgaaaaggt | 2881 |
| | ggctccgaga agtttttaca aagaacatgc tcacatcttc aggtgcctca ttcacatacg | 2941 |
| | tcaaggaaat tgaggtctgg aggcggctgg tggaaatcca attccccgcg gagcatggct | 3001 |
| | ggaaggagtc gttgctggga gacatggaat ggaggctcac aaaggaggaa cccctctccc | 3061 |
| | agatcactgc ctactgcaat agttgctggg acaccaaagg cttagaggac agtgtggcca | 3121 |
| | agaccttcga gaaatgcatc attgaagccg tgagctcagc ctgccaggtg aacaatctct | 3181 |
| | cctcctggga aacggattcg ggctcacagc tgtgttctgc catgacccag ctaagggcta | 3241 |
| | tgaagcaccc gctgggtctc agctcctccg ctaactcaga gattgggaag tgggcaccct | 3301 |
| | cctccctcgc caagggcaat ggcgctgaaa tctagttctc tccggattcc tcagtgtgct | 3361 |
| | gcacagtccc tgctgctcgc accatcctgc atgtgttcca tatggaatca cggccgtgcg | 3421 |
| | cgtgtggcac aagtcacacg ggcttgcagg ccgttcctca gatggccctg tcatcactgt | 3481 |
| | ggctgctggt ttgattgatt gttaacactt gctcagtagg tgtgcgggaa gagactccaa | 3541 |
| | aggttgacag aacatttatg gaagcaaaat atgtgaaatg gaaaattgta tcaatttatt | 3601 |
| | tagctctttt cggcaaaggg gaagagattg tgcccccctg tctcccagga acagtctcgc | 3661 |
| | aggcaatgcc acatgaggaa gctccctgct ggccacggct gccctgctca catttcctaa | 3721 |
| | ttggacactt aacccctgta caagcacagc cttgcggcca caggggaagt ccagaaacat | 3781 |
| | tgaggtcatt gaattccggg gaccaagggg ttctaatttt ttaagtgact gatacctttg | 3841 |
| | ataaggtttt cctttccctt tttcgttaac tctttgttga gatattgttc gtatgccata | 3901 |
| | cggtacatct gtcaaaagtt ccagttcagc aggttttggg gtagtcacag atatgtacag | 3961 |
| | tcatcaccac agttaatgac agagcatttt catcacttca aagagaaacc cggccccttt | 4021 |
| | agccatcatc ctcctcccct ctagtcaccc actcccctct gcaggcataa acaattgctg | 4081 |
| | aacataaaca actgcttctg tggctttctc tgttctgact gtcatatgaa tggaatcata | 4141 |
| | tcatatgtgg ccttttgggt atggcttatt tcactgagca taatgttttt ttgttgttgg | 4201 |
| | tggtggtggt tgtttgttgt ttttgagaca gagtttcact ctttttgccc aggctggagt | 4261 |
| | gcaatggtgc gatcctggct caccgcaacc gctgcctccc gggttcaagt gattctcctg | 4321 |
| | cctcagcctc ccaagtgctg gaattacagc tactttttgt ttaaagagtc ttttaattgt | 4381 |
| | ttaaagaaca atgtgccgcc acactcggtt ccttttgtat ttttagaaga gacagggttt | 4441 |
| | ctccatgttg gtcaggctgg tctcgaactc ccgacctcag gtgatccacc caccttggcc | 4501 |
| | tcccgaagtg ccgagattac aggtgtgagc caccgcgccc ggccgagcat aatgttttga | 4561 |
| | aagaccgctc aggctggaca cggttgctca cgcctgtaat cacggcactt tgggagccca | 4621 |
| | ggagttcaag acaagcctgg gtaacagagt aagaccctgt ctctataaaa actaaaaaat | 4681 |
| | aaacaaaaaa aattagccag gcatggccgg gcacacctgt ggtcccagct acttgggagg | 4741 |
| | ctgaggtggg aggctccctt gggcccagaa ggtcaaggca gcagtgagcc atgatcacac | 4801 |
| | cactgcactt caacctgggg gacagagcaa gaccctgtct caaaaagcaa taacaacaaa | 4861 |
| | agtccatcca tattgtagct tgtgtccgtt tacgttaagt attccttacc caaaatgctt | 4921 |
| | gagaccagaa gtgttttgga tttcagatgt tttcaaattt tggaatattt gcatttacat | 4981 |
| | aatgagatgt cttccagatg ggacccagag tctaaccaca aaattcactt gtttcatata | 5041 |
| | catcttatac acatagcctg aaggtaattt tatacaatat ttttaacaat tttgtgcatg | 5101 |
| | agacaaagtt tgtattaagt atttgtatgt tgaatttttc acttgtggca tcattatact | 5161 |
| | caaaaagttt gtgttttgga gtattttgga tttggggatt aggaatgctc aacctatatt | 5221 |
| | tcattttttt ccatggccaa atattccccg tttatccatg tgtccattga cggccatcta | 5281 |
| | tgttgcttct tcggctatta taaatctgct gatacaaaaa aaaaaa | |

Homo sapiens S100 calcium binding protein A8 (S100A8), mRNA
NCBI Reference Sequence: NM_002964.3 (SEQ ID. NO. 51)

| | | |
|---|---|---|
| 1 | atgtctcttg tcagctgtct ttcagaagac ctggtggggc aagtccgtgg gcatcatgtt | |
| 61 | gaccgagctg gagaaagcct tgaactctat catcgacgtc taccacaagt actccctgat | 121 |
| | aaaggggaat ttccatgccg tctacaggga tgacctgaag aaattgctag agaccgagtg | 181 |
| | tcctcagtat atcaggaaaa agggtgcaga cgtctggttc aaagagttgg atatcaacac | 241 |
| | tgatggtgca gttaacttcc aggagttcct cattctggtg ataaagatgg gcgtggcagc | 301 |
| | ccacaaaaaa agccatgaag aaagccacaa agagtagctg agttactggg cccagaggct | 361 |
| | gggcccctgg acatgtacct gcagaataat aaagtcatca atacctcaaa aaaaaaaaaa | 421 |
| | aaaaaaaa | |

Homo sapiens matrix metallopeptidase 7 (matrilysin, uterine) (MMP7), mRNA
NCBI Reference Sequence: NM_002423.3 (SEQ ID. NOs. 52, 53)

| | | |
|---|---|---|
| 1 | accaaatcaa ccataggtcc aagaacaatt gtctctggac ggcagctatg cgactcaccg | |
| 61 | tgctgtgtgc tgtgtgcctg ctgcctggca gcctggccct gccgctgcct caggaggcgg | 121 |
| | gaggcatgag tgagctacag tgggaacagg ctcaggacta tctcaagaga ttttatctct | 181 |
| | atgactcaga aacaaaaaat gccaacagtt tagaagccaa actcaaggag atgcaaaaat | 241 |
| | tctttggcct acctataact ggaatgttaa actcccgcgt catagaaata atgcagaagc | 301 |
| | ccagatgtgg agtgccagat gttgcagaat actcactatt tccaaatagc ccaaaatgga | 361 |
| | cttccaaagt ggtcacctac aggatcgtat catatactcg agacttaccg catattacag | 421 |
| | tggatcgatt agtgtcaaag gctttaaaca tgtggggcaa agagatcccc ctgcatttca | 481 |
| | ggaaagttgt atggggaact gctgacatca tgattggctt tgcgcgagga gctcatgggg | 541 |
| | actcctaccc atttgatggg ccaggaaaca cgctggctca tgcctttgcg cctgggacag | 601 |
| | gtctcggagg agatgctcac ttcgatgagg atgaacgctg gacggatggt agcagtctag | 661 |
| | ggattaactt cctgtatgct gcaactcatg aacttggcca ttctttgggt atgggacatt | 721 |
| | cctctgatcc taatgcagtg atgtatccaa cctatggaaa tggagatccc caaaatttta | 781 |
| | aactttccca ggatgatatt aaaggcattc agaaactata tggaaagaga agtaattcaa | 841 |
| | gaaagaaata gaaacttcag gcagaacatc cattcattca ttcattggat tgtatatcat | 901 |
| | tgttgcacaa tcagaattga taagcactgt tcctccactc catttagcaa ttatgtcacc | 961 |
| | cttttttatt gcagttggtt tttgaatgtc tttcactcct tttaaggata aactccttta | 1021 |
| | tggtgtgact gtgtcttatt catctatact tgcagtgggt agatgtcaat aaatgttaca | 1081 |
| | tacacaaata aataaaatgt ttattccatg gtaaatttaa aaaaaaaaaa aaaaaaaaaa | 1141 |
| | aaaaaaa | |

Homo sapiens small proline-rich protein 2A (SPRR2A), mRNA
NCBI Reference Sequence: NM_005988.2 (SEQ ID. NO. 54)

| | | |
|---|---|---|
| 1 | aaacccctgg tacctgagca ctgatctgcc ttggagaacc tgatcctgag actccagcag | |
| 61 | gatgtcttat caacagcagc agtgcaagca gccctgccag ccacctcctg tgtgccccac | 121 |
| | gccaaagtgc ccagagccat gtccaccccc gaagtgccct gagccctgcc caccaccaaa | 181 |
| | gtgtccacag ccctgcccac ctcagcagtg ccagcagaaa tatcctcctg tgacaccttc | 241 |
| | cccaccctgc cagtcaaagt atccaccgaa gagcaagtaa cagcttcaga attcatcagg | 301 |
| | accaagaaag gataaggata tttggctcac ctcgttccac agctccacct tcatcttctc | 361 |
| | atcaaagcct accatggata cacagggagc ttctttctcc ttagccagta atctgcccat | 421 |
| | gatgatccct gacagcaaaa agtttctttt ctgaggctgc catactgcca ctgtccaggt | 481 |
| | ggagactgag caaaggaagt cctgggctgt gccagctccc agagcttcgg aagaaagagc | 541 |
| | agcagctctc tccctgggaa ccatcagaga attctgttga tgtgttctgt gtctgtctgt | 601 |
| | cacctggtca cgagcttcta ccacctttgc aattgtcact tatctttcac tccctgaata | 661 |
| | aagtatctat gcatataaaa aaaaaaaaaa | |

Homo sapiens gap junction protein, beta 2, 26 kDa (GJB2), mRNA
NCBI Reference Sequence: NM_004004.4 (SEQ ID. NO. 55)

| | | |
|---|---|---|
| 1 | ggggtgcggt taaaaggcgc cacggcggga gacaggtgtt gcggccccgc agcgcccgcg | |
| 61 | cgctcctctc cccgactcgg agcccctcgg cggcgcccgg cccaggaccc gcctaggagc | 121 |
| | gcaggagccc cagcgcagag accccaacgc cgagaccccc gccccggccc cgccgcgctt | 181 |
| | cctcccgacg cagagcaaac cgcccagagt agaagatgga ttggggcacg ctgcagacga | 241 |
| | tcctgggggg tgtgaacaaa cactccacca gcattggaaa gatctggctc accgtcctct | 301 |
| | tcatttttcg cattatgatc ctcgttgtgg ctgcaaagga ggtgtgggga gatgagcagg | 361 |
| | ccgactttgt ctgcaacacc ctgcagccag gctgcaagaa cgtgtgctac gatcactact | 421 |
| | tccccatctc ccacatccgg ctatgggccc tgcagctgat cttcgtgtcc acgccagcgc | 481 |
| | tcctagtggc catgcacgtg gcctaccgga gacatgagaa gaagaggaag ttcatcaagg | 541 |
| | gggagataaa gagtgaattt aaggacatcg aggagatcaa aacccagaag gtccgcatcg | 601 |
| | aaggctccct gtggtggacc tacacaagca gcatcttctt ccgggtcatc ttcgaagccg | 661 |
| | ccttcatgta cgtcttctat gtcatgtacg acggcttctc catgcagcgg ctggtgaagt | 721 |
| | gcaacgcctg gccttgtccc aacactgtgg actgctttgt gtcccggccc acggagaaga | 781 |
| | ctgtcttcac agtgttcatg attgcagtgt ctggaatttg catcctgctg aatgtcactg | 841 |
| | aattgtgtta tttgctaatt agatattgtt ctgggaagtc aaaaaagcca gtttaacgca | 901 |
| | ttgcccagtt gttagattaa gaaatagaca gcatgagagg gatgaggcaa cccgtgctca | 961 |
| | gctgtcaagg ctcagtcgct agcatttccc aacacaaaga ttctgacctt aaatgcaacc | 1021 |
| | atttgaaacc cctgtaggcc tcaggtgaaa ctccagatgc cacaatggag ctctgctccc | 1081 |
| | ctaaagcctc aaaacaaagg cctaattcta tgcctgtctt aattttcttt cacttaagtt | 1141 |
| | agttccactg agaccccagg ctgttagggg ttattggtgt aaggtacttt catattttaa | 1201 |
| | acagaggata tcggcatttg tttctttctc tgaggacaag agaaaaaagc caggttccac | 1261 |
| | agaggacaca gagaaggttt gggtgtcctc ctggggttct ttttgccaac tttccccacg | 1321 |
| | ttaaaggtga acattggttc tttcatttgc tttggaagtt ttaatctcta acagtggaca | 1381 |
| | aagttaccag tgccttaaac tctgttacac tttttggaag tgaaaacttt gtagtatgat | 1441 |
| | aggttatttt gatgtaaaga tgttctggat accattatat gttccccctg tttcagaggc | 1501 |
| | tcagattgta atatgtaaat ggtatgtcat tcgctactat gatttaattt gaaatatggt | 1561 |
| | cttttggtta tgaatacttt gcagcacagc tgagaggctg tctgttgtat tcattgtggt | 1621 |
| | catagcacct aacaacattg tagcctcaat cgagtgagac agactagaag ttcctagtga | 1681 |
| | tggcttatga tagcaaatgg cctcatgtca aatatttaga tgtaattttg tgtaagaaat | 1741 |
| | acagactgga tgtaccacca actactacct gtaatgacag gcctgtccaa cacatctccc | 1801 |
| | ttttccatga ctgtggtagc cagcatcgga aagaacgctg atttaaagag gtcgcttggg | 1861 |
| | aattttattg acacagtacc atttaatggg gaggacaaaa tggggcaggg gagggagaag | 1921 |
| | tttctgtcgt taaaaacaga tttggaaaga ctggactcta aagtctgttg attaaagatg | 1981 |
| | agctttgtct acttcaaaag tttgtttgct taccccttca gcctccaatt ttttaagtga | 2041 |
| | aaatatagct aataacatgt gaaaagaata gaagctaagg tttagataaa tattgagcag | 2101 |
| | atctatagga agattgaacc tgaatattgc cattatgctt gacatggttt ccaaaaaatg | 2161 |
| | gtactccaca tatttcagtg agggtaagta ttttcctgtt gtcaagaata gcattgtaaa | 2221 |
| | agcattttgt aataataaag aatagcttta atgatatgct tgtaactaaa ataattttgt | 2281 |
| | aatgtatcaa atacatttaa aacattaaaa tataatctct ataataattt (SEQ ID NO: 192) | |
| 1 | accaggcaac accattgaag gctcatatgt aaaaatccat gccttccttt ctcccaatct | |
| 61 | ccattcccaa acttagccac tggcttctgg ctgaggcctt acgcatacct cccggggctt | |
| 121 | gcacacacct tcttctacag aagacacacc ttgggcatat cctacagaag accaggcttc | |
| 181 | tctctggtcc ttggtagagg gctactttac tgtaacaggg ccagggtgga gagttctctc | |
| 241 | ctgaagctcc atcccctcta taggaaatgt gttgacaata ttcagaagag taagaggatc | |
| 301 | aagacttctt tgtgctcaaa taccactgtt ctcttctcta ccctgcccta accaggagct | |
| 361 | tgtcacccca aactctgagg tgatttatgc cttaatcaag caaacttccc tcttcagaaa | |
| 421 | agatggctca ttttccctca aaagttgcca ggagctgcca agtattctgc caattcaccc | |
| 481 | tggagcacaa tcaacaaatt cagccagaac acaactacag ctactattag aactattatt | |
| 541 | attaataaat tcctctccaa atctagcccc ttgacttcgg atttcacgat ttctcccttc | |
| 601 | ctcctagaaa cttgataagt ttcccgcgct tccctttttc taagactaca tgtttgtcat | |
| 661 | cttataaagc aaaggggtga ataaatgaac caaatcaata acttctggaa tatctgcaaa | |
| 721 | caacaataat atcagctatg ccatctttca ctattttagc cagtatcgag ttgaatgaac | |
| 781 | atagaaaaat acaaaactga attcttccct gtaaattccc cgttttgacg acgcacttgt | |
| 841 | agccacgtag ccacgcctac ttaagacaat tacaaaaggc gaagaagact gactcaggct | |
| 901 | taagctgcca gccagagagg gagtcatttc attggcgttt gagtcagcaa agaagtcaag | |
| 961 | atggccaaag ttccagacat gtttgaagac ctgaagaact gttacagtga aaatgaagaa | |
| 1021 | gacagttcct ccattgatca tctgtctctg aatcagaaat ccttctatca tgtaagctat | |
| 1081 | ggcccactcc atgaaggctg catggatcaa tctgtgtctc tgagtatctc tgaaacctct | |
| 1141 | aaaacatcca agcttacctt caaggagagc atggtggtag tagcaaccaa cgggaaggtt | |
| 1201 | ctgaagaaga gacggttgag tttaagccaa tccatcactg atgatgacct ggaggccatc | |
| 1261 | gccaatgact cagaggaaga aatcatcaag cctaggtcag caccttttag cttcctgagc | |
| 1321 | aatgtgaaat acaactttat gaggatcatc aaatacgaat tcatcctgaa tgacgccctc | |
| 1381 | aatcaaagta taattcgagc caatgatcag tacctcacgg ctgctgcatt acataatctg | |
| 1441 | gatgaagcag tgaaatttga catgggtgct tataagtcat caaaggatga tgctaaaatt | |
| 1501 | accgtgattc taagaatctc aaaaactcaa ttgtatgtga ctgcccaaga tgaagaccaa | |
| 1561 | ccagtgctgc tgaaggagat gcctgagata cccaaaacca tcacaggtag tgagaccaac | |
| 1621 | ctcctcttct tctgggaaac tcacggcact aagaactatt tcacatcagt tgcccatcca | |
| 1681 | aacttgttta ttgccacaaa gcaagactac tgggtgtgct tggcaggggg gccaccctct | |
| 1741 | atcactgact ttcagatact ggaaaaccag gcgtaggtct ggagtctcac ttgtctcact | |
| 1801 | tgtgcagtgt tgacagttca tatgtaccat gtacatgaag aagctaaatc ctttactgtt | |
| 1861 | agtcatttgc tgagcatgta ctgagccttg taattctaaa tgaatgttta cactctttgt | |
| 1921 | aagagtggaa ccaacactaa catataatgt tgttatttaa agaacaccct atattttgca | |
| 1981 | tagtaccaat cattttaatt attattcttc ataacaattt taggaggacc agagctactg | |
| 2041 | actatggcta ccaaaaagac tctacccata ttacagatgg gcaaattaag gcataagaaa | |
| 2101 | actaagaaat atgcacaata gcagttgaaa caagaagcca cagacctagg atttcatgat | |
| 2161 | ttcatttcaa ctgtttgcct tctactttta agttgctgat gaactcttaa tcaaatagca | |
| 2221 | taagtttctg ggacctcagt tttatcattt tcaaaatgga gggaataata cctaagcctt | |
| 2281 | cctgccgcaa cagtttttta tgctaatcag ggaggtcatt ttggtaaaat acttcttgaa | |
| 2341 | gccgagcctc aagatgaagg caaagcacga aatgttattt tttaattatt atttatatat | |
| 2401 | gtatttataa atatatttaa gataattata atatactata tttatgggaa ccccttcatc | |
| 2461 | ctctgagtgt gaccaggcat cctccacaat agcagacagt gttttctggg ataagtaagt | |
| 2521 | ttgatttcat taatacaggg cattttggtc caagttgtgc ttatcccata gccaggaaac | |
| 2581 | tctgcattct agtacttggg agacctgtaa tcatataata aatgtacatt aattaccttg | |
| 2641 | agccagtaat tggtccgatc tttgactctt ttgccattaa acttacctgg gcattcttgt | |
| 2701 | ttcaattcca cctgcaatca agtcctacaa gctaaaatta gatgaactca actttgacaa | |
| 2761 | ccatgagacc actgttatca aaactttctt ttctggaatg taatcaatgt ttcttctagg | |
| 2821 | ttctaaaaat tgtgatcaga ccataatgtt acattattat caacaatagt gattgataga | |
| 2881 | gtgttatcag tcataactaa ataaagcttg caacaaaatt ctctgacaaa aaaaaaaaaa | |
| 2941 | aaa | |

Homo sapiens interleukin 1, alpha (IL1A), mRNA.
ACCESSION NM_000575 (SEQ ID NO: 193)

| | |
|---|---|
| 1 | agttaggagg gccccgcctt ccccagctgc atataaaggt ctctggggtt ggaggcagcc |
| 61 | acagcacgct ctcagccttc ctgagcacct ttccttcttt cagccaactg ctcactcgct |
| 121 | cacctccctc cttggcacca tgaccacctg cagccgccag ttcacctcct ccagctccat |
| 181 | gaagggctcc tgcggcatcg gaggcggcat cgggggcggc tccagccgca tctcctccgt |
| 241 | cctggccgga gggtcctgcc gtgcccccag cacctacggg ggcggcctgt ctgtctcctc |
| 301 | tcgcttctcc tctgggggag cctgcgggct ggggggcggc tatggcggtg gcttcagcag |
| 361 | cagcagcagc tttggtagtg gcttcggggg aggatatggt ggtggccttg gtgctggctt |
| 421 | cggtggtggc ttgggtgctg gctttggtgg tggttttgct ggtggtgatg ggcttctggt |
| 481 | gggcagtgag aaggtgacca tgcagaacct caatgaccgc ctggcctcct acctggacaa |
| 541 | ggtgcgtgct ctggaggagg ccaacgccga cctggaagtg aagatccgtg actggtacca |
| 601 | gaggcagcgg cccagtgaga tcaaagacta cagtccctac ttcaagacca tcgaggacct |
| 661 | gaggaacaag atcattgcgg ccaccattga gaatgcgcag cccattttgc agattgacaa |
| 721 | tgccaggctg gcagccgatg acttcaggac caagtatgag catgaactgg ccctgcggca |
| 781 | gactgtggag gccgacgtca atggcctgcg ccgggtgttg gatgagctga ccctggccag |
| 841 | gactgacctg gagatgcaga tcgaaggcct gaaggaggag ctggcctacc tgaggaagaa |
| 901 | ccacgaggag gagatgcttg ctctgagagg tcagaccggc ggagatgtga acgtggagat |
| 961 | ggatgctgca cctggcgtgg acctgagccg catcctgaat gagatgcgtg accagtacga |
| 1021 | gcagatggca gagaaaaacc gcagagacgc tgagacctgg ttcctgagca agaccgagga |
| 1081 | gctgaacaaa gaagtggcct ccaacagcga actggtacag agcagccgca gtgaggtgac |
| 1141 | ggagctccgg agggtgctcc agggcctgga gattgagctg cagtcccagc tcagcatgaa |
| 1201 | agcatccctg gagaacagcc tggaggagac caaaggccgc tactgcatgc agctgtccca |
| 1261 | gatccaggga ctgattggca gtgtggagga gcagctggcc cagctacgct gtgagatgga |
| 1321 | gcagcagagc caggagtacc agatcttgct ggatgtgaag acgcggctgg agcaggagat |
| 1381 | tgccacctac cgccgcctgc tggagggcga ggatgcccac ctttcctccc agcaagcatc |
| 1441 | tggccaatcc tattcttccc gcgaggtctt cacctcctcc tcgtcctctt cgagccgtca |
| 1501 | gacccggccc atcctcaagg agcagagctc atccagcttc agccagggcc agagctccta |
| 1561 | gaactgagct gcctctacca cagcctcctg cccaccagct ggcctcacct cctgaaggcc |
| 1621 | cgggtcagga ccctgctctc ctggcgcagt tcccagctat ctcccctgct cctctgctgg |
| 1681 | tggtgggcta ataaagctga ctttctggtt gatgcaaaaa |

Homo sapiens keratin 16 (KRT16), mRNA.
NM_005557 (SEQ ID NO: 194)

| | |
|---|---|
| 1 | gatagaccat gagcagccat ggcaacagcc tgttccttcg ggagagcggc cagcggctgg |
| 61 | gccgggtggg ctggctgcag cggctgcagg aaagcctgca gcagagagca ctgcgcacgc |
| 121 | gcctgcgcct gcagaccatg accctcgagc acgtgctgcg cttcctgcgc cgaaacgcct |
| 181 | tcattctgct gacggtcagc gccgtggtca ttggggtcag cctggccttt gccctgcgcc |
| 241 | catatcagct cacctaccgc cagatcaagt acttctcttt tcctggagag cttctgatga |
| 301 | ggatgctgca gatgctggtg ttacctctca ttgtctccag cctggtcaca ggtatggcat |
| 361 | ccctggacaa caaggccacg gggcggatgg ggatgcgggc agctgtgtac tacatggtga |
| 421 | ccaccatcat cgcggtcttc atcggcatcc tcatggtcac catcatccat cccgggaagg |
| 481 | gctccaagga ggggctgcac cgggagggcc ggatcgagac catccccaca gctgatgcct |
| 541 | tcatggacct gatcagaaat atgtttccac caaaccttgt ggaggcctgc ttcaaacagt |
| 601 | tcaagacgca gtacagcacg agggtggtaa ccaggaccat ggtgaggaca gagaacgggt |
| 661 | ctgagccggg tgcctccatg cctcctccat tctcagtgga gaacggaacc agcttcctgg |
| 721 | aaaatgtcac tcgggccttg ggtaccctgc aggagatgct gagctttgag gagactgtac |
| 781 | ccgtgcctgg ctccgccaat ggcatcaacg ccctgggcct cgtggtcttc tctgtggcct |
| 841 | ttgggctggt cattggtggc atgaaacaca agggcagagt cctcagggac ttcttcgaca |
| 901 | gcctcaatga ggctattatg aggctggtgg gcatcattat ctggtatgca cctgtgggca |
| 961 | tcctgttcct gattgctggg aagattctgg agatggaaga catggccgtc ctggggggtc |
| 1021 | agctgggcat gtacaccctg accgtcatcg tgggcctgtt cctccatgcc ggcattgtcc |
| 1081 | ttcccctcat ctacttcctc gtcactcacc ggaacccctt ccccttcatt gggggcatgc |
| 1141 | tacaagccct catcaccgct atgggcacgt cttccagctc ggcaacgctg cccatcacct |
| 1201 | tccgctgcct ggaggagggc ctgggtgtgg accgccgcat caccaggttc gtcctgcccg |
| 1261 | tgggcgccac ggtcaacatg gatggcactg ccctctacga ggccctggct gccatcttca |
| 1321 | ttgctcaagt taacaactac gagctcaacc tgggtcagat cacaaccatc agcatcacgg |
| 1381 | ccacagcagc cagtgttggg gctgctggca tcccccaggc gggtctggtc accatggtca |
| 1441 | ttgtgcttac gtcggtcggc ttgcccacgg aagacatcac gctcatcatc gccgtggact |
| 1501 | ggttccttga ccggcttcgc acaatgacca acgtactggg ggactcaatt ggagcggccg |
| 1561 | tcatcgagca cttgtctcag cgggagctgg agcttcagga agctgagctt accctcccca |
| 1621 | gcctggggaa accctacaag tccctcatgg cacaggagaa gggggcatcc cggggacggg |
| 1681 | gaggcaacga gagtgctatg tgaggggcct ccagctctg |

Homo sapiens solute carrier family 1 (high affinity
aspartate/glutamate transporter), member 6 (SLC1A6), mRNA.
ACCESSION NM_005071 (SEQ ID NO: 195)

TABLE 6
AMINO ACID SEQUENCES

```
>gi|4504411|ref|NP_002115.1| major histocompatibility complex, class II, DR
beta 1 precursor [Homo sapiens]
MVCLKLPGGSCMTALTVTLMVLSSPLALAGDTRPRFLWQLRFECHFFNGTERVRLLERCIYNQEESVRFD
SDVGEYRAVTELGRPDAEYWNSQKDLLEQRRAAVDTYCRHNYGVGESFTVQRRVEPKVTVYPSKTQPLQH
HNLLVCSVSGFYPGSIEVRWFRNGQEEKAGVVSTGLIQNGDWTFQTLVMLETVPRSGEVYTCQVEHPSVT
SPLTVEWRARSESAQSKMLSGVGGFVLGLLFLGAGLFIYFRNQKGHSGLQPTGFLS (SEQ ID NO: 100)
>gi|4504577|ref|NP_002155.1| indoleamine 2,3-dioxygenase 1 [Homo sapiens]
MAHAMENSWTISKEYHIDEEVGFALPNPQENLPDFYNDWMFIAKHLPDLIESGQLRERVEKLNMLSIDHL
TDHKSQRLARLVLGCITMAYVWGKGHGDVRKVLPRNIAVPYCQLSKKLELPPILVYADCVLANWKKKDPN
KPLTYENMDVLFSFRDGDCSKGFFLVSLLVEIAAASAIKVIPTVFKAMQMQERDTLLKALLEIASCLEKA
LQVFHQIHDHVNPKAFFSVLRIYLSGWKGNPQLSDGLVYEGFWEDPKEFAGGSAGQSSVFQCFDVLLGIQ
QTAGGGHAAQFLQDMRRYMPPAHRNFLCSLESNPSVREFVLSKGDAGLREAYDACVKALVSLRSYHLQIV
TKYILIPASQQPKENKTSEDPSKLEAKGTGGTDLMNFLKTVRSTTEKSLLKEG (SEQ ID NO: 88)
>gi|4504931|ref|NP_002272.1| keratin, hair, basic, 1 [Homo sapiens]
MTCGSGEGGRAFSCISACGPRPGRCCITAAPYRGISCYRGLTGGEGSHSVCGGFRAGSCGRSEGYRSGGV
CGPSPPCITTVSVNESLLTPLNLEIDPNAQCVKQEEKEQIKSLNSRFAAFIDKVRFLEQQNKLLETKLQF
YQNRECCQSNLEPLFEGYIETLRREAECVEADSGRLASELNHVQEVLEGYKKKYEEEVSLRATAENEFVA
LKKDVDCAYLRKSDLEANVEALIQEIDFLRRLYEEETRILQSHISDTSVVVKLDNSRDLNMDCIIAEIKA
QYDDIVTRSRAEAESWYRSKCEEMKATVIRHGETLRRTKEEINELNRMIQRLTAEVENAKCQNSKLEAAV
AQSEQQGEAALSDARCKLAELEGALQKAKQDMACLIREYQEVMNSKLGLDIEIATYRRLLEGEEQRLCEG
IGAVNVCVSSSRGGVVCGDLCVSGSRPVTGSVCSAPCNGNVAVSTGLCAPCGQLNTTCGGGSCGVGSCGI
SSLGVGSCGSSCRKC (SEQ ID NO: 61)
>gi|4505187|ref|NP_002407.1| C-X-C motif chemokine 9 precursor [Homo sapiens]
MKKSGVLELLGITLINLIVQGTPVVRKGRCSCISTNQGTIHLQSLKDLKQEAPSPSCEKIETIATLKNC
VQTCLNPDSADVNELIKKWEKQVSQKKKUNGKKHQKKKVLKVRKSQRSRQKKTT (SEQ ID NO: 59)
>gi|4505219|ref|NP_002414.1| matrilysin preproprotein [Homo sapiens]
MRLTVLCAVCLLPGSLALPIPQEAGCMSELQWEQAQDYLKRFYLYDSETKNANSLEAKLKEMQKFFGLPI
TGMLNSRVIEIMQKPRCGVPDVAEYSLFPNSPKWTSKVVTYR1VSYTRDLPHITVDRLVSKALNMWGKEI
PLHERKVVWGTADIMIGEARGAHGDSYPEDGPGNTLAHAPAPGTGLGGDAHEDEDERWTDGSSLGINFLY
AATHELGHSLGMGHSSDPNAVMYPTYGNGDPQNFKLSQDDIKGIQKLYGKRSNSRKK (SEQ ID NO: 107)
>gi|4505219|ref|NP_002414.1| matrilysin preproprotein [Homo sapiens]
MRLTVLCAVCLLPGSLALPIPQEAGGMSELQWEQAQDYLKRFYLYDSETKNANSLEAKLREMMEGLPI
TGMLNSRVIEIMQKPRCGVEDVAEYSLEPNSPKWTSKVVTYRIVSYTRDLPHITVDRINSKALNMWGKEI
PLHFRKVVWGTADIMIGFARGAHODSYPEDGPGNTLAHAFAPOTGLOGDAHEDEDERWTDOSSLGINFLY
AATHELGHSLOMGHSSDPNAVMYPTYGNGDPQNFKLSQDDIKGIQKLYGKRSNSRKK (SEQ ID NO: 108)
>gi|4505787|ref|NP_002629.1| elafin preproprotein [Homo sapiens]
MRASSFLIVVVFLIAGTLVLEAAVTGVEWKGQDTVKGRVPFNGQDPVKGQVSVKGQDKVKAUPVKGPVS
TKPGSCPIILIRCAMLNPPNRCLKDTDCPGIKKCCEGSCGMACFVPQ (SEQ ID NO: 74)
>gi|4506851|ref|NP_002984.1| C-X-C motif chemokine 6 [Homo sapiens]
MSLPSSRAARVPGPSGSIZALLALLLLLTPPGPLASAGPVSAVLTELRCTCLRVTLRVNPKTIGKLQVFP
AGPQCSKVEVVASLKNOKQVCLDPEAPFLKKVIQKILDSGNKKN (SEQ ID NO: 80)
>gi|4507925|ref|NP_003871.1| WNT1-inducible-signaling pathway protein 3
isoform 1 [Homo sapiens]
MOLLFSTLLLAGLAQFCCRVOGTGPLDTTPEGRPGEVSDAPQRKUCHWPCKCPQQKPRCPPOVSLVRD
GCGCCKICAKQPGETCNEADLCDPHKGLYCDYSVDRPRYETGVCAYLVAVGCEFNQVHYHNGOVFONPL
FSCLCVSGAIGCTPLFIPKLAGSHCSOAKGGKKSDQSNCSLEPLLQQLSTSYKTMPAYRNLPLIWKKKCL
VQATKWTPCSRTCOMGISNRVTNENSNCEMRKEKRLCYIQPCDSNILKTIKTPKGKTCQPTFQLSKAEKF
VFSGCSSTOSYKPTFCGICLDKRCCIPNKSKMITTQFDCPNEGSFKWKMLWITSCVCQRNCREPGDIFSE
LKIL (SEQ ID NO: 66)
>gi|4757734|ref|NP_004824.1| interferon-inducible protein AIM2 [Homo sapiens]
MESKYKEILLLTOLDNITDEELDRFKFFLSDEFNIATGKLEITANHIQVATLMIQNAGAVSAVMKTIRIFQ
KLNYMLLAKRLQEEKEKVDKQYKSVTKPKPLSQAEMSPAASAAIRNDVAKQRAAPKVSPHVKPEQKQMVA
QQESIREGFQKRCLPVMVLKAKKPFTFETQEGKQEMEHATVATEKFFFFVKVFNTLLKDKFIPKRIIIIA
RYYRHSGFLEVNSASRVLDAESDOKVNVPLNIIRKAGETPKINTLOTOPLGTIVNGLEVVQKVTEKKKNI
LFDLSDNTGKMEVLGVRNEDTMKCKEGDKVRLTFFTLSKNGEKLOLTSGVHSTIKVIKAKKKT (SEQ ID
NO: 85)
>gi|4758494|ref|NP_004122.1| granzyme 13 precursor [Homo sapiens]
MQPILLLLAFLLLPRADA-6EIIGGHEAKPHSRPYMAYLMIWDOKSLKRCOGFLIQDDEVLTAAHCWOSSI
NVTLGAHNIKEQEPTQUIPVKRPIPHPAYNPKNESNDIMLLQLERKAKRTRAVULRLPSNKAQVKIDGQ
TCSVAGWGQTAPLOKHSHTWEVKMTVQEDRKCESDLRHYYDSTIELCVGDPEIKKTSFKODSOCPLVCN
KVAQGIVSYCRNNGMPPRACTKVSSFVHWIKKTMKRY (SEQ ID NO: 95)
>gi|4885111|ref|NP_005176.1| calmodulin-like protein 3 [Homo sapiens]
MADOLTEEQVTEFKEAFSEFDKDODGCITTRELGTVMRSLGQNPTEAELRDMMSEIDRDONGTVDFPEFL
GMMARKMKDTDNEEEIREAFRVEDKDGNOFVSAAELRHVMTRLGEKLSDEEVDEMIRAADTDGDGQVNYE
EFVRVLVSK (SEQ ID NO: 78)
>gi|5031839|ref|NP_005545.1| keratin, type II cytoskeletal 6A [Homo sapiens]
MASTSTTIRSHSSSRROF-S-ANSARLPOVSRSGESSVSVSRSRGSGOLOGACGGAGEGSRSLYGLGGSKRI
STOGGSCAISGGYGSRAGGSYGEGGAGSGFGEGGGAGIGFOLOGGAGLAGGEGGPGFETCPPGGIQEVTV
NOSLLTPLNLQIDPTIQRVRAEEREQIKTLNNKFASFIDKVRFLEQQNKVLETKWTIALQFQGTKTVROL
EPLFEQYINNLRRQLDSIVGERGRLDSELROMQDLVEDEKNKYEDEINKRTAAENEFVTLKKDVDAAYMN
KVELQAKADTLTDEINFLRALYDAELSQMOTHISDTSVVLSMDNNRNLDLDSIIAEVKAWEEIAQRSRA
EAESWYOTKYEELQVTAGRHGDDLRNTKQEIAEINRMIQRLRSEIDHVKKOCANLQAAIADAEQRGEMAL
KDAKNKLEGLEDAWKAKQDLARLLKEYQELMNVKLALDVEIATYRKLLEGEECRLNGEGVGQVNISVW
STVSSOYGGASGVGSGLOLGGGSSYSYGSOLGVGGGESSSSGRAIGGOLSSVGCOSSTIKYTTTSSSSRK
SYKH (SEQ ID NO: 98)
>gi|5174693|ref|NP_005979.1| small proline-rich protein 2A [Homo sapiens]
MSYQQQQCKQPCQPPPVC-FTPKCPEPCPPPKCPEPCPPPKCPQPCPPQQCQQKYPPVTPSPPCOSKYITK
SK (SEQ ID NO: 109)
>gi|5454144|ref|NP_006389.1| ubiquitin D [Homo sapiens]
MAFNASCLCVHVRSEEWDEMTFDANPYDSVKKIKEHVRSKTKVIWODQVULGSKILKPRRSLSSYGIDK
EKTIHLTLKVVKPSDEELPLELVESODEAKRHLLQVRRSSSVAQVKAMIFTKTGIIPETQIVTCNGKRLE
DOKMMADYGIRKGNLLFLASYCIGG (SEQ ID NO: 84)
>gi|5902072|ref|NP_008850.1| serpin B3 [Homo sapiens]
MNSLSEANTKFMFDLFQQFRKSKENNIFYSPISITSALGMVLLGAKDNTAQQIKKVLHFDQVTENTTGKA
ATYHVDRSGNVHHQFQKLLTEFNKSTDAYELKIANKLFGEKTYLFWEYLDAIKKEYQTSVESVDFANAP
EESRKKINSWVESQTNEKIKNLIPEGNIGSNTTLVLVNAIYFKGQWEKKFNKEDTKEEKEWPNKNTYKSI
QMMRWTSFHFASLEDWAKVLEIPYKGKDLSMIVLLPNEIDGLQKLEEKLTAEKLMEWTSLQNMRETRV
DLHLPREKVEESYDLKDTLRTMGMVDIFNGDADLSGMTGSRGLVLSGVLHKAFVEVTEEGAEAAAATAVV
GFGSSPTSTNEEFHCNHPFLFFIRQNKTNSILFYGRFSSP (SEQ ID NO: 79)
>gi|7108346|ref|NP_036615.1| granulysin isoform 519 [Homo sapiens]
MEGINFSRLSPEYYDPARABLRDGEKSCPCGQEGPQGDLLTKTQELGRDYRTCLTIVOKLKKMVDKPTQR
SVSNAATRVCRTGRSRWRDVCRNFMRRYQSRVIQGLVAGETAQQICEDLRLCIPSTGPL (SEQ ID NO: 72)
>gi|8393956|ref|NP_036529.1| serpin B13 [Homo sapiens]
MDSLGAVSTRLGFDLFKEEKKTNDGNIFFSPVGILTAIGMVLLGTRGATASQLEEVFHSEKETKSSRIKA
EEKEVIENTEAVHQQFQKFLTEISKLTNDYELNITNRLFGEKTYLFLOKYLDYVEKYYHASLEPVDEVNA
ADESRKKINSWVESKTNEKIKDLFPDGSISSSTKINLVNMVYFKGQWDREFKKENTKEEKEWMNKSTSKS
VQMMTQSHSFSFTFLEDLOAKILGIPYKNNDLSMFVLLPNDIDOLEKIIDKISPEKLVENTSPGHMEERK
VNLHLPRFEVEDGYDLEAVLAAMCMCDAFSEHKADYSGMSSGSGLYAQKFIASSEVAVTEEGTEAAAATG
IGFTVTSAPGHENVHCNHPFLFFIRHNESNSILFFGRFSSP (SEQ ID NO: 87)
>gi|10567820|ref|NP_066386.1| melanoma-associated antigen 10 [Homo sapiens]
MPRAPKRQRCMPEEDLQSQSETQCLEGAQAPLAVEEDASSSTSTSSSFPSSFPSSSSSSSSSCYPLIPST
PEEVSADDETPNPPQSAWACSSPSVVASLPLDQSDEGSSSUEESPSTLQVLPDSESLPRSEIDEKVTD
LVQFLLFKYQMKEPITKAEILESVIKNYEDHFPLLFSEASECMLLVFGIDVKEVDPTGHSFVLVTSLGLT
YDGMLSDVQSMPKTGILILILSIIFIEGYCTPEEVIWEALNMMGLYDGMEHLIYGEPRKLLTQDWVQENY
LEYRQVPGSDPARYEFLWGPRAHAEIRKMSLLKFLAKVNGSDPRSFPLWYEEALKDEEERAQDRIATTDD
TTAMASASSSATGSFSYPE (SEQ ID NO: 56)
>gi|10947122|ref|NP_064693.1| ATP-binding cassette, sub-family C, member 9
isoform SUR2E [Homo sapiens]
MSLSFCGNNISSYNINDGVLQNSCFVDALNLVPHVFLLFITFPILFIGWGSQSSKVQIHHNTWLHFPGHN
LRWILTFALLFVHVCEIAEGIVSDSRRESRHLHLEMPAVMGFVATTTSIVYYHNIETSNFPKLLLALFLY
WVMAFITKTIKLVKYCQSGLDISNLRFCITGMMVILNGLLMAVEINVIRVRRYVFFMNPQKVICPPEDLQD
LGVRFLUFVNLLSKATYWWMNTLIISAHKKPIDLKAIGKLPIAMRAVTNYVCLKDAYEEQKKKVADHPN
RTPSIWLAMYRAFGRPILLSSTFRYLADLLGFAGPLCISGIVQRVNETQNGTNNTTGISETLSSKEFLEN
AYVLAVLLFLALILORTFWASYYVTIETGINLRGALLAMIYNKILRLSTSNLSMGEMTLGQINNLVAIE
TNOLMWELFLCPNLWAMPVQIIMGVILLYNLLGSSALVGAAVIVLLAPIQYFIATKLAEAQKSTLDYSTE
RLKKTNEILKGIKLLKLYAWEHIECKSVEETRMKELSSLKTFALYTSLSIFMNAAIPIAAVLATFVTHAY
ASGNNLKPAEAFASLSLFHILVTPLSLLFTVVRFAVKAIISVQKLNEFLLSDEIGDDSWRTGESSLPFES
CKKHTGVQPKTINRKQPGRYHLDSYEQSTRRLRPAETEDTAIKVTNGYFSWGSGLATLSNIDIRIPTGQL
TMIVGQVGCGKSSLLLATLGEMQTLEGKVHWSNVNESEPSFEATRSRNRYSVAYAAOKPWLLNATVEENI
TEGSPFNKQRYKAVTDACSLUDIDLLPFGDQTEIGERGINLSGGQRQRICVARALYQNTNIVFLDDPFS
ALDIHLSDHLMQEGILKFLODDKRTLVINTHKLQYLTHADWIIAMKDGSVLREGTLKDIQTKDVELYEHW
KTLMNRQDQELEKDMEADQTTLERKTLRRAMYSREAKAQMEDEDEEEEEEEDEDDNMSTVMRLRTKMPWK
TCWRYLTSGGEFLLILMIFSKLLKHSVIVAIDYWLATWTSEYSINNTGKADQTYYVAGFSILCGAGIFLC
INTSLTVEWMGLTAAKNLHHNLLNKIILGPIRFFDTTPLGLILNRFSADTNIIDQHIPPTLESLTRSTLL
CLSAIGMISYATPVFLVALLPLGVAFYFIQKYFRVASKDLQELDDSTQLPLLCHFSETAEGLTTIRAFRH
ETRFKQRMLELTDTNNIAYLFLSAANRWLEVRTDYLCACIVLTASIASISGSSNSGLVGLGLLYALTITN
YLNWVVRNLADLEVQMGAVKKVNSFLTMESENYEGTMDPSUPEHWPOEGEIKIHDLCVRYENNLKPVLK
HVKAYIKPGQKVGICGRTGSGKSSLSLAFFRMVDIFDGKIVIDGIDISKLPLHTLRSRLSIILQDPILFS
GSTRFNLDPECKCTDDRLWEALEIAOLKNMVKSLPGGLDAVVTEGGENFSVGQRQLFCLARAFVRKSSIL
IMDEATASIDMATENILQKVVMTAFADBTVVTMAHRVHTILTADLVIVMKRGNILEYDTPESLLAQENGV
FASEVRADM (SEQ ID NO: 86)
>gi|15431310|ref|NP_000517.21 keratin, type I cytoskeletal 14 sapiens)
MTTCSRQFTSSSSMKESCGiGGGIGGGSSRISSVLAGGSCRAPSTYGGGLSVSSSRFSSGGAYGLGGGYG
GGFSSSSSSEGSGEGGGYGGGLGAGLGGGEGGGFAGGDGLLVGSEKVTMOLNDRLASYLDKVRALEEAN
ADLEVKIRDWYQRQRPAEIKDYSPYEKTIEDLRNKILTATVDNANVLLQIDNARLAADDERTKYETELNL
RMSVEADINGLRRVLDELTLARADLEMQIESLKEELAYLKKNHEEEMNALRGQVGGDVNVEMDAAPGVDL
SRILNEMROQYEKMAEKNRKDAEEWPFTKTEELNREVATNSELVQSGKSEISELRRTMQNLEIELQSQLS
MKASLENSLEETKGRYCMQLAQIQEMIGSVEEQLAQLRCEMEQQNQEYKILLDVKTRLEQETATYRRLLE
GEDAHLSSSUSSOSQSSRDVTSSSRQIRTKVMDVHDGKVVSTHEQVLRTKN (SEQ ID NO: 91)
>gi|16418425|ref|NP 443174.1| guanylate-binding protein 5 [Homo sapiens]
MALEIHMSDPMCLIENFNEQLKVNQEALEILSAITUVVVVAIVGLYRTGKSYLMNKLAGKNKGESVAST
VQSHTKGIWIWCVPHPNWPNHTLVLLDTEGLGDVEKADNKNDINFALALLLSSTEVYNTVNKIDQGAID
LLHNVTELTDLLKARNSPDLDRVEDPADSASEEPDLVWTLRDFCLGLEIDGQLVTPDEYLENSLRPKQGS
DQRVQNFNLPRLCIUFFPKKKCETEDLPAHQKKLAQLETLPDDELEPEFVQQVTEECSYIESHSMTKTI,
PGGIMVNGSRLKNLVLTYVNAISSGDLPCIENAVLALAQRENSAAVQKAIAHYDQQMGQKVQLPMETLQE
LLDLHRTSEREAIEVEMKNSFKDVDQSFQKELETLLOAKQNDICKRNLEASSDYCSALLKDIEGPLEEAV
KWIYSKPGGHNLFIQKTEELKAKYYREPRKGIQAEEVLQKYLKSKESVSHAILOTDQALTETEKKKKEA
QVKAEAEKAEAQRLAAIQRQNEQMMQERERLHQEQVROMEIAKOWLAEQQKMQEQQMQEQAAQLSTTFQ
AQNRSLLSELQHAQRTVNNDDPCVLL (SEQ ID NO: 69)
>gi|21071008|ref|NP_001053.21 transcobalamin-1 precursor [Homo sapiens]
MROSHQLPINGULFSFIPSQLCEICEVSEENYIRLKPLLNTMIQSNYNRGTSAVNVVLSLKLVGIQIQT
LMQKMIQQZKYNVKSRLSDVSSGELALIILALGVCRNAEENLIYDYHLIDKLENKFQAEIENMEAHNGTP
LTNYYQLSLDVLALCLENGNYSTAEVVNHETPENKNYYFGSQFSVDTGAMAVLALTCVMSLINGQIKAD
EGSLKNISIYTKSLVEKILSEKKENGLIGNTESTGEAMOALEVSSDYYNENDWNCQQTLNTVLTETSQGA
FSNPNAAAQVLPALMGKTFLDINKDSSCVSASGNENTSADEPTTVTPPDSQSYTSVNYSVRINETYETNV
TVLNGSVELSVMEKAUMNDTIFGETMEERSWGPYITCIQOLCANNNDRTYWELLSGGEPLSQGAGSYVV
RNGENLEVRWSKY (SEQ ID NO: 82)
>gi|21361559|ref|NP_003376.21 visinin-like protein 1 [Homo sapiens]
MGKONSKLAPEVMEDLVKSTEENEHELKQWYKGELKDCPSGRLNLEEFINLYVKFEPYGDASKFAQHAFR
TEDKNGDGTIDEREFICALSITSRGSFEQKLNWAFNMYDLDGDGKITRVEMLEIIEAIYKMVGTVIMMKM
NEDGLTPEQRVDKIFSKMDKNKDDQITLDEFEEAAKSDPSIVLLLQCDIQK
>gi|21389379|ref|NP 653195.1| gametocyte specific factor 1 [Homo sapiens]
MEETYTDSLDPEKLLQCPYDKNHQIRACREPYHLIKCRKNHPDVASKLATCPFNARHQVPRAEISHHISS
CDDRSCIEQDVVNQTRSLRQETLAESTWQCPPCDEDWDKDLWEQTSTPFAWGTTHYSDNNSPASNIVTEH
KNNLASGMRVPKSLPYVLPWKNNGNAQ (SEQ ID NO: 73)
>gi|21614544|ref|NP_002955.21 protein S100-A8 [Homo sapiens]
MLTELEKALNSIIDVYHKY-S-LIKGNEHAVYRDDLKKLLETECPQYIRKKGADVWFKELDINTDGAVNEQE
FLILVIKMGVAAHKKSHEESHKE (SEQ ID NO: 106)
>gi|28076869|ref|NP_002965.1| serpin B4 [Homo sapiens]
MNSLSEANTKFMFDLEQURKSKENNIFYSPISITSALGMVLLGAKDNTAQQISKVLHEDQVTENTTEKA
ATYHVDRSGNVHHQFQKLLTEENKSTDAYELKIANKLFGEKTYQFWEYLDAIKKEYOTSVESTDFANAP
EESRKKINSWVESONEKIKNLEPDGTIGNDTTLVLVNAIYFKGQWENKFKKENTKEEKEWPNKNTYKSV
QMMRUNSENFALLEDVQAKVLEIPYKGKDLSMIVLLPNEIDGLQKLEEKLTAEKLMEWTSLQNMRETCV
DLHLPRFKMEESYDLKDTLRTMGMVNIENGDADLSGMTWSHGLSVSKVLHKAFVEVTEEGVEAAAATAVV
VVELSSPSTNEEFCCNHPFLFFIRQNKTNSILFYGRESSP (SEQ ID NO: 71)
>gi|28827815|ref|NP_789793.1| protein S100-A7A [Homo sapiens]
MSNTQAERSIIGMIDMFHKYTORDOKTEKPSLLTMMKENFPNELSACDKKGIHYLATVFEKKDKNEDKKI
DESEFLSLLGDTAADYHKQSHGAAPCSGGSQ (SEQ ID NO: 75)
>gi|29150261|ref|NP_006323.21 gamma-interferon-inducible lysosomal thiol
reductase preproprotein [Homo sapiens]
MTLSPLLIJELPPLULLDVPTAAVQASPLQALDFFGNGPPVNYKTGNLYLRGPLKKSNAPINNVTLYYEA
LOGGCRAFLIRELEPTWLLVMEILNVTLVPYGNAQEQNVSGRWEEKCQHGEEECKENKVEACVLDELDME
LAFLTIVCMEEFEDMERSLPLCLQLYAPGLSPDTIMECAMGDRGMQLMHANAQRTDALQPPHEYVPWVTV
NGKPLEDQTQLLTLVCQLYQGKKPDVCPSSTSSLRSVCFK (SEQ ID NO: 103)
>gi|32313593|ref|NP_006409.31 olfactomedin-4 precursor [Homo sapiens]
MRPGLSELLALLEFLGQAA6DLGDVGPPIPSPGESSFPGVDSSSSESSSSRSGSSSSRSLGSGGSVSQLF
SNFTGSVDDRGTCQCSVSLPDTTFPVDRVERLEFTAHVLSQKFEKELSKVREYVQLISVYEKKLLNLTVR
IDTMEKDTISYTELDFELIKVEVIKEMEKLVIQLKESEGGSSEIVDQLEVEIRNMTLLVEKLETLDKNNVL
AIRREIVALKTKLKECEASKDQNTPVVHPPPTPGSCGBGGVVNISKPSVVQLNWRGESYLYGAWGRDYSP
QHPNKGLYWVAPLNTDGRLLEYYRLYNTLDDLLLYINARELRITYGQGSGTAVYNNNMYVNMYNTGNIAR
VNLTTNTIAVTOLPNAAYNNRFSYANVAWOIDFAVDENGLWVIYSTEASTGNMVISKLNDTTLQVLNT
WYTKUKPSASNAFMVCGVLYATRTMNTRTEEIFYYYDTNIGKEGKLDIVMHKMQEKVQSINYNPFDQKL
YVYNDGYLLNYDLSVLQKPQ (SEQ ID NO: 81)
>gi|38455402|ref|NP_005555.21 neutrophil gelatinase-associated lipocalin
precursor [Homo sapiens]
MPLGLLWLGLALLGALHAQAODSTSDLIPAPPLSKVPLQQNFQDNQFQGKWYVVGLAGNAILREDKDPQK
MYATIYELKEDKSYNVISVLERKKKCDYWIRTFVPGCQPGEFTLGNIKSYPGLTSYLVRVVSTNYNQHAM
VFEKKVSQNREYFKITLYGRTKELTSELKENFIRFSKSLGLPENHIVEPVPIDQCIDG (SEQ ID NO: 101)
>gi|39995089|ref|NP_945315.1| parathyroid hormone-related protein isoform 2
preproprotein [Homo sapiens]
MQRRINQQWSVAVELLSYAVPSCGRSVEGLSRRLKRAVSEHQLLHDKGKSIQDLRRRFELHHLIAEIHTA
EIRATSEVSPNSKPSPNTKNHPVREGSDDEGRYLTUTNKVETYKEULKTPGKKKKGKPGKRKEQEKKK
RRTRSAWLDSGVTGSGLEGDHLSDTSTTSLELDSR (SEQ ID NO: 63)
>gi|40254837|ref|NP_006774.21 gap junction beta-6 protein [Homo sapiens]
MDWGTLHTFIGGVNKHSTSIGKWITVIFIERVMILVVAAQEVWGDEQEDEVCNTLQPGCKNVCYDHFFP
VSHIRLWALQLIFVSTPALLVAMHVAYYRHETTRKERRGEKRNDETDIEDIKKQKVRIEGSIMWTYTSSI
FFRIIFEAAFMYVEYFLYNGYHLPWVLKCGTDPCPNINDCFISRPTEKTVETIFMISASVICMLLNVAEL
CYLLIWCFRRSKRAQTQKNHPNHALKESKONEMNELISDSGWAITGEPS (SEQ ID NO: 77)
>gi|40254997|ref|NP_116288.21 hypothetical protein LOC84985 isoform a
[Homo sapiens]
MSRSRHLGKIRKRLEDVKSQWVRPARADFSDNESARLATDALLOGGSEAYWRVLSQEGEVDEISSVEAQY
IQAQAREPPCPPDTLOGAEAGPKGLDSSSLQSGTYFPVASEGSEPALLHSWASAEKPYLKEKSSATVYFQ
TVKHNNIRDTAVRRCITRTSQVIMILMDANTDVEIFCDILEAANKRGVENCVLLDQGGVKLEQEMCDKVQI
SDSHLKNISIRSVEGEIYCAKSGRKFAGQIREKFITSDWRFVLSGSYSFTWLOGHVHRNILSKFTWAVE
LEDEEFRHLYASSKEWMGLKSPRLVAPVPPGAAPANGRLSSSSGSASDRTSSNPFSGRSAGSHPGTRSVS
ASSGPCSPAAPHPPPPPREQPHQGPWGAPSPQAHLSPRPHDGPPAAVYSNLGAYRPTRLQI,EQLGINPRL
TPTWRPFLQASPHE (SEQ ID NO: 92)
>gi|42558283|ref|NP_003995.21 gap junction beta-2 protein [Homo sapiens]
MDWGTLQTILGOVNISHSTSYGKIWLTVLFIFRIMILVVAAKEVWGDEQADFVCNTLQPGCKNVCYDHYFP
ISHIRLWALQLIFVSTPALINAMHVAYRRHEKKRKFIKGEIKSEEKDIEETKTUVRIEGSLWWTYTSSI
FERVIFEAAFMYVEYVMYDGFSMQRINKCNAWPCPNTVDCFVSRPTEKTVFTVFMIAVSGICILLNWEL
CYLLIRYCSGKSKEKPV (SEQ ID NO: 110)
>gi|44680117|ref|NP_982252.1| Down syndrome critical region gene 8 isoform b
[Homo sapiens]
MKEPGPNFVTVRKGLHSFKMAFVKHLLULETKIWLE (SEQ ID NO: 57)
>gi|44680119|ref|NP_982253.1| Down syndrome critical region gene 8 isoform c
[Homo sapiens]
MKEPGPNFVTVRKGLHSFKMAFVKHLL (SEQ ID NO: 60)
>giJ54873602|ref|NP_787081.21 hypothetical protein LOC220382 [Homo sapiens]
MAVQAALLSTHETVPMFGdSPDGLOGAFGALDKGCCFEDDETGAPAGALLSGAEGGDVREATHDLLSFI
DSASSNIKLALDKPOKSKRKVNHRKYLOWIKRCSGLMGAAPPGPMPSAADTPAKRPLAAPSAPTVAAP
AHOKAAPRREAWAAAAASLORSLAALFDSLRHVPGGAEPAGGEVAAPAAGIAGGAGTOGAGODVAGPAG
ATAIPGARKVPLRARNLPPSFFTEPSRAGGGGCGPSGPDVSLODLEKGAEAVEFFELIAGPDYGAGTEAAV
LLAAEPLDVFPAGASVLRGPPELEPOLFEPPPAVVONLLYPEPWSVPGCSPTKKSPLTAPRGGLTLNEPL
SPLYPAAADSPOGEDGRGHLASPAPFFPDCALPPPPPPHIVSYDYSAGYSRTAYSSIMSDGVWEGAPGE
EGAHRD (SEQ ID NO: 93)
>gi|66529203|ref|NP_066005.21 protein AL017 isoform 2 [Homo sapiens]
MECPSCQHVSKEETPKFCSOGERLPPAAPIADSENNNSTMASASEGEMECGQELKEEGGPCLEPGSDSW
OENPEEPCSKASWTWESKKKKRKKKKKGNKSASSELASLPLSPASPCHLTLLSNMPQDTALPHSQAQQ
SOPTGQPSUPGTATTPLEGDGLSAPTEVGDSPLQAQALGEAGVATGSEAQSSPQFQDHTEGEDQDASIP
SGGRGLSQEGTGPPTSAGEGHSRTEDAAQELLLPESMOSSEPGTELOTTEQQAGASASMAVDAVAEPAN
AVKGAGKEMKEKTQRMKUPATTPPFKTHCQEAETKTKDEMAAAEEKVGKNEQGEPEDLKKPEGKNRSAA
AVKNEKEQKNQEADVQEVKASTLSPGGGVTVFFHAIISLHFPPNETLHKVFIRGGEEFGESKIIDSNICEL
HYTRDLGHDRVLVEGIVCISKKHLDKYIPYKYVIYNGESFEYEFIYKHQQKKGEYVNRCLFIKSSLLGSG
DWHQYYDIVYMKPHGRLQKVMNHITDOPRKDLVKGKQTAAALMLDSTFSILQTWDTINLNSETTQFEQFC
FVLQIUMIYEGQAQTAWTDLUREKEVICRYLWQHLKKHVVPLPDGKSTDELPVDCPVRSKLKTGLIVLFVV
EKIELLLEGSLDWLCHLLTSDASSPDEFHRDLSHILGIPOWRLYLVNLCQRCMDTRTYTWLGALPVTAHC
CMELAPRHKDAWRQPEDTWAALEGLSFSPFREQMLDTSSLLQFMREKQHIALSIDEPLFRSWFSLLPLSHL
VMYMENFIEHLGREPAHILDCLSGITYRLPOLEWLNTQDVQDVQNVQNILEMLLRLI,DTYRDKIPEEAL
SPSYLTVCLKLHEAICSSTEMLKFYELPALSAEIVCRMIRLI,SLVDSAGQRDETGNNSVQTVFQGTLAAT
KRWLREVFTKNMLTSSGASFTYVKEIEVWRRINEIQFPAEHOWKESLLGDMEWRLTKEEPLSQITAYCNS
CWDTKGLEDSVAKTFEKCIIEAVSSACQVNNIASSWETDSGSQLCSAMTQLRAMKHPLGLSSSANSEIGKW
APSSLAKONGAEI (SEQ ID NO: 105)
>gi|73858572|ref|NP_002417.21 macrophage metalloelastase preproprotein
[Homo sapiens]
MKFLLITALQATASGALPLNSSTSLEKNNVLFGERYLEKEYGLEINKLEWTKMKYSGNI,MKEKWEMQHF
LGLKVTGQLDTSTLEMMHAPRCGVPDVHHFREMPGGPVWRKHYTTYRINNYTPDMNREDVDYAIRKAFQV
WSNVTPLKFSKINTGMADILVVFARGAHODFHAFDGKGGILAHAFGPGSGIGGDAHFDEDEFWTTHSGGT
NLFLTAVHEIGHSLOLGHSSDPKAVMETTYKYVDINTFRLSADDIRGIOSLYGDPKENQRLPNETNSEPA
LCDPNLSFDAVTTVGNKIFFFKDRFFWLKVSERPKTSVNLISSIMPTLPSGTEAAYEIEARNQVFLFKDD
KYWLISNLRPEPNYPKSIHSFGFPNFVKKIDAAVFNPRFYRTYFFVDNQYWRYDERRQMMDPGYPKLITK
NFQGIGPKIDAVFYSKNKYYYFFQGSNQFEYDFLDDRITKTLKSNSWFGC (SEQ ID NO: 58)
>gi|l09255249|ref|NP_002263.21 keratin, type II cytoskeletal 4 [Homo sapiens]
MTSVGVESDMLNGCGKDGIXT'RAKPRDVSDFSLYAPATKPCCSRTYKRRRLRAPALTGLGPVTSLIAPSS
LSAAMIARQQCVROGPROFSCGSATVGGGECRGAFSSVSMSGGAGRCSSGGEGSRSLYNLRGNKSISMSVA
GSRWACFGGAGGFGTGGFGGGFGGSFSGKGGPGFPVCPAGGIOEVTINQSLLTPLHVEIDETIQKVRTE
EREQIKLLNNKFASFIDKVQFLEQQNKVLETKWNLLQWTTTTSSKNLEPLFETYLSVLRKQLDTLGNDK
GRWSELKTWDSVEDFKTKYEEEINKRTAAENDEVVLKKDVDAAYLNKVELEAKVDSLNDEINELKVLY
DAELSQMQTHVSDTSVVLSMDNNRNLDLDSIIAEVRAINEEIAQRSKAEAEALYQTKVOQLQISVDQHGD
NLKNTKSEIAELNRMIQRLRAEIENIKKOCQTLWSVADAEQRGENALKDAHSKRVELEAALQQAKEELA
RMLREYQELMSVKLALDIEIATYRKLLEGEEYRMSGECQSAVSISVVSGSTSTGGISCGLGSGSGFGLSS
GFGSGSGSGFGEGGSVSGSSSSKIISTTTLNKRR (SEQ ID NO: 102)
>gi|113416509|ref|XP_001131447.1| PREDICTED: hypothetical protein
[Homo sapiens]
MASAARRSSGRHTSRPTTPGAAQRRCVLAALRGFRRGPAGLGRETRVPAGAGLGDATAAISHRGGVGKRG
SLRLQGLSTASOQPQQRPPVSAGQRARPVPRPPSSSAGPGPEGPEGAGCVLRLSAISAGPELRETHELLE
(SEQ ID NO: 62)
>gi|115298657|ref|NP_002954.21 protein S100-A7 [Homo sapiens]
MSNTQAERSIIGMIDMEHKYYRRDDKTEKPSLLTMMKENFPNFLSACDKKGTNYLADVFEKKDKNEDKKI
DESEFLSLLODIATDYHKQSHGAAPCSGGSQ (SEQ ID NO: 65)
>gi|119395754|ref|NP_000415.21 keratin, type II cytoskeletal 5 [Homo sapiens]
MSRQSSVSERSGGSRSFSTASAITPSVSRTSFISVSRSCGGCCGOFCRVSLAGAGOVGGYGSRSLYNLGG
SKRISISTSGGSFRNREGAGAGGGYGEGGGAGSGFOFGGGAOGGFOLGGGAGEGGOEGGPGFPVCPPGGI
QEVTVNQSLLTPLNLQIDPSIQRVRTEEREQIKTLNNKFASEIDKVRFLEQQNKVLDTKWTLLQEQGTKT
VRQNLEPLFEQYINNLRRQLDSTVGERGRLDSELRNMOLVEDEKNKYEDEINKRTTAENEFVMLKKDVD
AAYMNKVELEAKVDALMDEINFMKMFFDAELSQMQTHVSDTSVVLSMDNNRNLDLDSIIAEVKAWEEIA
NRSRTEAESWYOTKYEELQUAGRHGDDLRNTKHEISEMNRMIQRLRAEIDNVKKQGANLQNAIADAEQR
GELALKDARNKLAELEEALQKAKQDMARLLREYQELMNTKLALDVEIATYRKLLEGEECRLSGEGVGPVN
ISVVTSSVSSUGSGSCYGGGLGGGLGGGLGGGLAGGSSGSYYSSSSGGVGLGGGLSVGGSGESASSGRG
LOVGFOSGGGSSSSVKEVSTTSSSRKSEKS (SEQ ID NO: 89)
>gi|119703753|ref|NP_005546.21 keratin, type II cytoskeletal 68
[Homo sapiens]
MASTSTTIRSHSSSRRGESANSARLPGVSRSGESSISVSRSRGSGGLGGACGGAGEGSRSLYGLCCSKRI
SIGGGSCAISGGYGSRAGGSYGEGGAGSGEGEGGGAGIGFCLCGGACLAGGEGOPCFPVCPPGGINVTV
NQSLLTPLNLQIDPAIQRVRAEEREQTKILNNKFASFIDKVRFLEQQNKVLDTKWTLLQEQGTKTVROL
EPLFEQYINNLRRQLDNIVGERGRLDSELRNMQDLVEDLKNKYEDEINKRTAAENEFVTLICKDVDAAYMN
KVELQAKADTLTDEINFLRALYDAELSQMOTHISDTSVVLSMDNNRNLDLDSIIAEVKAWEEIAQRSRA
EAESWYQTKYEELQITAGRHGDDLRNTKQEIAEINRMIQRLRSEIDHVKKQCANLQAAIADAEQRGEMAL
KDAKNKLECLEDAWKAKQDLARLLKEITELMNVKLALDVEIATYRKLLEGEECRLNGEGVGQVNISVW
STVSSGYGGASGVGSGLGLGGGSSYSYGSGLGVGGGESSSSGRATGGGLSSVGGGSSTIKYTTTSSSSRK
SYKH (SEQ ID NO: 99)
>gi|119964718|ref|NP_001935.21 desmoglein-3 preproprotein [Homo sapiens]
MMGLEPRTTGALAIFVVVILVHGELRIETKGQYDEEEMTMQQAKRRQKREWVKFAKPCREGEDNSKRNPI
AKITSDYQATUITYRISGVGIDQPPEGIFVVDKNTGDINITAIVDREETPSFLITCRALNAWLDVEKP
LILTVKILDINDNPPVESQQIFMGEIEENSASNSLVMILNATDADEPNHLNSKIAFKIVSQEPAGTPMFL
LSRNIGEVRTLTNSLDREQASSYRLVVSGADKDGEGLSTQCECNIKVKDVNDNFPMFRDSUSARIEENI
LSSELLRFUTDLDEEYTDNWLAVYFFTSONEONWEEIQTDPRTNEGILKVVKALDYEQLQSVKLSIAVK
NKAEFHQSVISRYRVQSTPVTIQVINVREGTAFRPASKTFTVQKGISSKKLVDYILGTWAIDEDTNKAA
SNVKYVMGRNDGGYLMIDSKTAEIKEVKNMNRDSTFIVNKTITAEVLAIDEYTGKTSTGTVYVRVPDFND
NCPTAVLEKDAVCSSSPSVVVSARTLNNRYTGPYTFALEDQPVKLPAVWSITTLNATSALLRAQEQIPPG
VYHISINLTDSONRCEMPRSLTLEVCQCDNRGIGGTSYPTTSPGTRYGRPHSGRLGPAAIGLLLLGLLL
LLIJAPLLLLICDCGAGSTCOVTGGFIPVPDGSEGTIHOWGIEGAHPEDKEITNICVPPVTANGADEMESS
EVCINTYARGTAVEGTSGMEMITKLGAATESGGAAGFATGTVSGAASGFGAATGVGICSSGQSGTMRTRH
STGGTNKDYADGAISMNFLDSYESQKAFACAEEDDGQEANDCLLIYDNEGADATGSPVCSVOCCSEIADD
LDDSELDSLGPKEKKLAEISLGVDGECKEVQPPSKDSGYGIESCGHPIEVQQTGFVKCQTLSGSQGASAL
STSGSWPAVSIPDPLUCNYLVTETYSASGSLVUSTAGFDPLLTQNVIVIERVICPISSVPGNLAGPT
QLRGSHTMLCTEDPCSRLI (SEQ ID NO: 96)
>gi|149999382|ref|NP_001556.21 C-X-C motif chemokine 10 precursor
[Homo sapiens]
MNQTAILICCLIFLTLSGIQGVPLSRTVRCTCISISNQPVNPRSLEKLEIIPASQFCPRVEIIATMKKKG
EKRCLNPESKAIKNLLKAVSKERSKRSP (SEQ ID NO: 67)
>gi|166158925|ref|NP_001107228.1| thymidine phosphorylase precursor
[Homo sapiens]
MAALMTPGTGAPPAPGDFSGEGSQGLPDPSPEPKQLPELIRMKRDGGRLSEADIRGEVAAVVNGSAQGAQ
IGAMLMAIRLRGMDLEETSVLTQALAQSGQQLEWPEAWRQQLVDKHSTGGVGDKVSLVLAPALAACGCKV
PMTSGRGLGHTGGTLDKLESIPGENVIQSPEQMQVLLDQAGCCIVGQSEQLVPADGILYAARDVTATVDS
LPLITASILSKKLVEGLSALVVDVKFGGAAVFPNQEQARELAKTINGVGASLGLRVAAALTAMDKPLGRC
VGHALEVEEALLCMDGAGPPDLRDLVTTLGGALLWLSGHAGTQAWAARVAAALDDGSALGRFERMLAAQ
GVDPGLARALCSGSPAERRQLLPRAREQEELLAPADGTVELVRALPLALVLHELGAGRSRAGEPLRLGVG
AELLVDVGQRLRRGTPWLRVHRDGPALSGPQSRALQEALVLSDRAPFAAPSPFAELVLPPQQ (SEQ ID NO: 97)
>gi|1691647831refj0001718550.1| PREDICTED: hypothetical protein [Homo sapiens]
MTPTLLLTVTVPRAAGSAGQRRAPGLPRSSGPAWAESRARPPRPRGLEPRHPPGSPALRPTDRTCSSSSA
GVGGGVGGAQPGSVPLGQHLALERGRTLGHGRVGRRDPPPLGLLVNPRVAGVDGLDRGGRLDPAGIGQVL
GLGVLGGAGRQRRALGGQALGLLAQVGIGAGHARGGRGAVGPAGQHRARLGAAVLRGTAGAPARRVGVVA
ERAASAACSLOQRLHARRRVREQRGRVAREVRGRVIGRGREVQPVVGRRHKPALRRGRARVLGLLRRQQ
PVGVRHAAVRTRPGARARARVEAGLGVVAHELVLQERAGHGVAGPGHDLRARRVVGRGGQAVHVTAGVDP
AGLFQKPLGKSRARSNHERLAFTRVLEPEVCCWKPPKYLVSIVSPV (SEQ ID NO: 90)
>gi|169204721|ref|XP_001713739.1| PREDICTED: hypothetical protein
[Homo sapiens]
MTCGENSIGCGFRPGNESCVSACGPRPSRCCITAAPYRGISCYRGLTGGEGSHSVCGGFRAGSCGRSEGY
RSGGVCGPSPPCITTVSVNESLLTPLNLEIDPNAQCVKQEEKEQIKSLNSRFAAFIDKVRFLEQQNKLLE
TKLUYQNRECCQSNLEPLFAGYIETLRREAECVEADSGRLASELNHVOEVLEGYKKRYEEEVALRATAE
NEEVALKKDVDCAYLRKSDLEANVEALIQEIDFLRRLYEEEIRILQSHISDTSVVVKLDNSRDLNMDCMV
AEIKAQYDDIATRSRAEAESWYRSKCEEMKATVIRHGETLRRTKEEINELNRMIQRLTAEVENAKCONSK
LEAAVAQSEQQGEAALSDARCKLAELEGALQKAKUMACLIREYQEVMNSKLGLDIEIATYRRLLEGEEH
RLCEGVEAVNVCVSSSRGGVVCGDLCVSGSRPVTGSVCSAPCNGNLVVSTGLCKPCGQLNTTCGGGSCGQ
GRY (SEQ ID NO: 104)
ILIA [Homo sapiens].
ACCESSION CAG33695
    1 MAKVPDMFED LKNCYSENEE DSSSIDHLSL NQKSFYHVSY GPLHEGCMDQ SVSLSISETS
  61 KTSKLTFKES MVVVATNGKV LKKRRLSLSQ SITDDDLEAI ANDSEEEIIK PRSAPFSFLS
121 NVKYNFMRII KYEFILNDAL NOSIIRANDQ YLTAAALHNL DEAVKFDMGA YKSSKDDAKI
181 TVILRISKTQ LYVTAQDEDQ PVLLKEMPEI PKTITGSETN LLFFWETHGT KNYFTSVAHP
241 NLFIATKQDY WVCLAGGPPS ITDFQILENQ A (SEQ ID NO: 166)
keratin, type I cytoskeletal 16 [Homo sapiens].
ACCESSION NP_005548
    1 MTTCSRQETS SSSMKGSCGI GGGIGGGSSR ISSVLAGGSC RAPSTYGGGL SVSSRFSSGG
  61 ACGLGGGYGG GFSSSSSFGS GFGGGYGGGL GAGFGGGLGA GEGGGFAGGD GLLVGSEKVT
121 MQNLNDRLAS YLDKVRALEE ANADLEVKIR DWYQRQRPSE IKDYSPYFKT IEDLRNKIIA
181 ATIENAQPIL QIDNARLAAD DFRTKYEHEL ALRQTVEADV NGLRRVLDEL TLARTDLEMQ
241 IEGLKEELAY LRKNHEEEML ALRGQTGGDV NVEMDAAPGV DLSRILNEMR DQYEQMAEKN
301 RRDAETWFLS KTEELNKEVA SNSELVQSSR SEVTELRRVL QGLEIELQSQ LSMKASLENS
361 LEETKGRYCM QLSQIQGLIG SVEEQLAQLR CEMEQQSQEY QILLDVKTRL EQEIATYRRL
421 LEGEDAHLSS QQASGQSYSS REVFTSSSSS SSRQTRPILK EQSSSSFSQG QSS (SEQ ID NO: 167)
solute carrier family 1 (high affinity aspartate/glutamate transporter),
member 6 [Bos taurus]
ACCESSION DAA28190
    1 MSSHGNSLFL RESGQRLGRV GWLQRLQESL QQRALRMRLR LQTMTREHVL RFLRRNAFIL
  61 LTVSAVVIGV SLAFALRPYQ LSYRQIKYFS FPGELLMRML QMLVLPLIVS SLVTGMASLD
121 NKATGRMGMR AAVYYMVTTV IAVFIGILMV TIIHPGKGSK EGLHREGRIE TIPTADAFMD
181 LVRNMFPPNL VEACFKQFKT QYSTRLVTRT VVRTDNGSEL GTSMPPLSSL ENGTGLLENV
241 TRALGTLQEV LSFEETVPVP GSANGINALG LVVFSVAFGL VIGGMKHKGR VLRDFFDSLN
301 EAIMRLVGII IWYAPVGILF LIAGKILEME DMAVLGGQLG MYTLTVIVGL FVHAGGILPL
361 IYFLITHRNP FPFIGGILQA LITAMGTSSS SATLPITFRC LEEGLGVDRR ITRFVLPVGA
421 TVNMDGTALY EALAAIFIAQ VNNYELNLGQ ITTISITATA AS (SEQ ID NO: 168)
```

TABLE 7

| Gene | GenBank (NCBI) Accession | FORWARD PRIMER | |
|---|---|---|---|
| Symbol | number | NAME | FORWARD PRIMER (5′->3′) |
| FCRLB | NM_001002901.2 | FCRLB-F1 | AGTGCAAGAGCTGTTCCGGGC (SEQ ID |
| | | | NO: 169) |
| IL1A | NM_000575.3 | UPL501_IL1A_F2 | GGTTGAGTTTAAGCCAATCCA (SEQ ID NO: 170) |
| KRT16 | NM_005557.3 | UPL509_KRT16-F1 | ATCGAGGACCTGAGGAACAA (SEQ ID NO: 171) |
| S100A2 | NM_005978.3 | S100A2-F1 | TCTGCCACCTGGTCTGCCACA (SEQ ID NO: 172) |
| S100A7A | NM_176823.3 | UPL507_S100A7A-F2 | AAGCCTGCTGACGATGATG (SEQ ID NO: 173) |
| SLC1A6 | NM_005071.1 | UPL511_SLC1A6-F1 | CTATGGGCACGTCTTCCAG (SEQ ID NO: 174) |
| KRT6A | NM_005554.3 | JK1186-KRT6A-F | TGAGGAGTGCAGGCTGAATGGC (SEQ ID |
| | | | NO: 175) |
| MMP12 | NM_002426.2 | JK1192-MMP12-F | TCTGGACTACACATTCAGGAGGCAC (SEQ ID |
| | | | NO: 176) |
| MMP11 | NM_005940.3 | JK1178-MMP11-F | ACCGCTGGAGCCAGACGCC (SEQ ID NO: 177) |
| COL10A1 | NM_000493.3 | ES577-COL10A1-F | GGGCCTCAATGGACCCACCG (SEQ ID NO: 178) |
| SFN | NM_006142.3 | JK1206-SFN-F | GTGGAGAGGGACTGGCAGAGC (SEQ ID |
| | | | NO: 179) |

TABLE 8

| Gene | GenBank (NCBI) Accession | | |
|---|---|---|---|
| Symbol | number | REVERSE PRIMER NAME | REVERSE PRIMER (5′->3′) |
| FCRLB | NM_001002901.2 | FCRLB-R1 | TACTCGGCGCCCCAGTCGAA (SEQ ID NO: 180) |
| IL1A | NM_000575.3 | UPL502_IL1A_R2 | TGCTGACCTAGGCTTGATGA (SEQ ID NO: 181) |
| KRT16 | NM_005557.3 | UPL510_KRT16-R1 | GGGCCAGTTCATGCTCATAC (SEQ ID NO: 182) |
| S100A2 | NM_005978.3 | S100A2-R1 | AGTGACCAGCACAGCCAGCG (SEQ ID NO: 183) |
| S100A7A | NM_176823.3 | UPL508_S100A7A-R2 | GCGAGGTAATGTATGCCCTTT (SEQ ID NO: 184) |
| SLC1A6 | NM_005071.1 | UPL512_SLC1A6-R1 | GGACGAACCTGGTGATGC (SEQ ID NO: 185) |
| KRT6A | NM_005554.3 | JK1187-KRT6A-R | CAATGGCTCTGCCACTGCTGGAAC (SEQ ID |
| | | | NO: 186) |
| MMP12 | NM_002426.2 | JK1193-MMP12-R | GTCACAGAGAGCTGGTTCTGAATTGTC (SEQ ID |
| | | | NO: 187) |
| MMP11 | NM_005940.3 | JK1179-MMP11-R | CGAGAGGCCAATGCTGGGTAGC (SEQ ID NO: 188) |
| COL10A1 | NM_000493.3 | ES578-COL10A1-R | CTGGGCCTTTGGCCTGCCTT (SEQ ID NO: 218) |
| SFN | NM_006142.3 | JK1207-SFN-R | GGGACACTCCTCAATTCCTACGATC (SEQ ID |
| | | | NO: 189) |

We claim:

1-13. (canceled)

14. A kit comprising a plurality of agents that bind to a plurality of markers chosen from COL10A1, FCRLB, AIM2, KRT6A, and MMP11.

15. The kit of claim 14 comprising a plurality of agents that binds to each of the following markers COL10A1, FCRLB, AIM2, KRT6A, and MMP11.

16. The kit of claim 14, wherein the plurality of agents are proteins and/or peptides.

17. The kit of claim 16, wherein the proteins are antibodies and/or antibody fragments.

18. The kit of claim 14, wherein the plurality of agents binds to COL10 and AIM2.

19. The kit of claim 14, wherein the plurality of agents are nucleic acid oligonucleotides.

20. The kit of claim 19, wherein the nucleic acid oligonucleotides are DNA oligonucleotides.

21. The kit of claim 19, wherein the nucleic acid oligonucleotides bind to a DNA sequence encoding a plurality of markers chosen from COL10A1, FCRLB, AIM2, KRT6A, and MMP11.

22. The kit of claim 15, wherein the plurality of agents are proteins and/or peptides.

23. The kit of claim 22, wherein the proteins are antibodies and/or antibody fragments.

24. The kit of claim 15, wherein the plurality of agents are nucleic acid oligonucleotides.

25. The kit of claim 24, wherein the nucleic acid oligonucleotides are DNA oligonucleotides.

26. The kit of claim 24, wherein the nucleic acid oligonucleotides bind to a DNA sequence encoding a plurality of markers chosen from COL10A1, FCRLB, AIM2, KRT6A, and MMP11.

27. The use of the kit of claim 14 to detect bladder cancer.

28. The use of the kit of claim 15 to detect bladder cancer.


SEQUENCE LISTING (208 sequences)

SEQ ID NO: 1 - DNA, 1519 bp, Homo sapiens
gagaagcgaggttctcgttctgagggacaggcttgagatcggctgaagagagcgggcccaggctctgtgaggaggcaagggaggtgagaaccttgctctcagagggtgactcaagtcaacacagggaacccctcttttctacagacacagtgggtcgcaggatctgacaagagtccaggttctcaggggacagggagagcaagaggtcaagagctgtgggacaccacagagcagcactgaaggagaagacctgcctgtgggtccccatcgcccaagtcctgcccacactcccacctgctaccctgatcagagtcatcatgcctcgagctccaaagcgtcagcgctgcatgcctgaagaagatcttcaatcccaaagtgagacacagggcctcgagggtgcacaggctcccctggctgtggaggaggatgcttcatcatccacttccaccagctcctcttttccatcctcttttccctcctcctcctcttcctcctcctcctcctgctatcctctaataccaagcaccccagaggaggtttctgctgatgatgagacaccaaatcctccccagagtgctcagatagcctgctcctccccctcggtcgttgcttcccttccattagatcaatctgatgagggctccagcagccaaaaggaggagagtccaagcaccctacaggtcctgccagacagtgagtctttacccagaagtgagatagatgaaaaggtgactgatttggtgcagtttctgctcttcaagtatcaaatgaaggagccgatcacaaaggcagaaatactggagagtgtcataaaaaattatgaagaccacttccctttgttgtttagtgaagcctccgagtgcatgctgctggtctttggcattgatgtaaaggaagtggatcccactggccactcctttgtccttgtcacctccctgggcctcacctatgatgggatgctgagtgatgtccagagcatgcccaagactggcattctcatacttatcctaagcataatcttcatagagggctactgcacccctgaggaggtcatctgggaagcactgaatatgatggggctgtatgatgggatggagcacctcatttatggggagcccaggaagctgctcacccaagattgggtgcaggaaaactacctggagtaccggcaggtgcctggcagtgatcctgcacggtatgagtttctgtggggtccaagggctcatgctgaaattaggaagatgagtctcctgaaatttttggccaaggtaaatgggagtgatccaagatccttcccactgtggtatgaggaggctttgaaagatgaggaagagagagcccaggacagaattgccaccacagatgatactactgccatggccagtgcaagttctagcgctacaggtagcttctcctaccctgaataaagtaagacagattcttcactgtgttttaaaaggcaagtcaaataccacatgattttactcatatgtggaatctaaaaaaaaaaaaaaaaaaa

SEQ ID NO: 2 - DNA, 441 bp, Homo sapiens
accccaccctaatcttgttatgcaaataggcttcccacttggcaggggccgtcttgtccactcgtttctgtaaacatgggtggcaaaaagagaagatggagctgccatttagaacatgcctaatcccagcttcatcttgctgagcaaaaatgaaggagcctggacccaactttgttactgtgagaaagggtcttcattcattcaagatggcatttgttaagcacctactacaaaccttggaaatcaagaaagttctggaatgatgaagctgttcatgccaagaccgaaagtgctggcccagtatgagtccattcagttcatgccgtgacaattttcttggaactcctttttattgttagttctcacttgtttccatatttagtgaatgtacatttaattgcaaagctgtcattaataaaaattcttatagtacctcaaaaa

SEQ ID NO: 3 - DNA, 1825 bp, Homo sapiens
agaaaggaacacagtaaactgaattgatccgtttagaagtttacaatgaagtttcttctaatactgctcctgcaggccactgcttctggagctcttcccctgaacagctctacaagcctggaaaaaaataatgtgctatttggtgaaagatacttagaaaaattttatggccttgagataaacaaacttccagtgacaaaaatgaaatatagtggaaacttaatgaaggaaaaaatccaagaaatgcagcacttcttgggtctgaaagtgaccgggcaactggacacatctaccctggagatgatgcacgcacctcgatgtggagtccccgatgtccatcatttcagggaaatgccaggggggcccgtatggaggaaacattatatcacctacagaatcaataattacacacctgacatgaaccgtgaggatgttgactacgcaatccggaaagctttccaagtatggagtaatgttacccccttgaaattcagcaagattaacacaggcatggctgacattttggtggtttttgcccgtggagctcatggagacttccatgcttttgatggcaaaggtggaatcctagcccatgcttttggacctggatctggcattggaggggatgcacatttcgatgaggacgaattctggactacacattcaggaggcacaaacttgttcctcactgctgttcacgagattggccattccttaggtcttggccattctagtgatccaaaggccgtaatgttccccacctacaaatatgttgacatcaacacatttcgcctctctgctgatgacatacgtggcattcagtccctgtatggagacccaaaagagaaccaacgcttgccaaatcctgacaattcagaaccagctctctgtgaccccaatttgagttttgatgctgtcactaccgtgggaaataagatctttttcttcaaagacaggttcttctggctgaaggtttctgagagaccaaagaccagtgttaatttaatttcttccttatggccaaccttgccatctggcattgaagctgcttatgaaattgaagccagaaatcaagtttttctttttaaagatgacaaatactggttaattagcaatttaagaccagagccaaattatcccaagagcatacattcttttggttttcctaactttgtgaaaaaaattgatgcagctgtttttaacccacgtttttataggacctacttctttgtagataaccagtattggaggtatgatgaaaggagacagatgatggaccctggttatcccaaactgattaccaagaacttccaaggaatcgggcctaaaattgatgcagtcttctactctaaaaacaaatactactatttcttccaaggatctaaccaatttgaatatgacttcctactccaacgtatcaccaaaacactgaaaagcaatagctggtttggttgttagaaatggtgtaattaatggtttttgttagttcacttcagcttaataagtatttattgcatatttgctatgtcctcagtgtaccactacttagagatatgtatcataaaaataaaatctgtaaaccataggtaatgattatataaaatacataatatttttcaattttgaaaactctaattgtccattcttgcttgactctactattaagtttgaaaatagttaccttcaaaggccaagagaattctatttgaagcatgctctgtaagttgcttcctaacatccttggactgagaaattatacttacttctggcataactaaaattaagtatatatattttggctcaaataaaattgaaaaaaaaatca

SEQ ID NO: 4 - DNA, 2545 bp, Homo sapiens
atccaatacaggagtgacttggaactccattctatcactatgaagaaaagtggtgttcttttcctcttgggcatcatcttgctggttctgattggagtgcaaggaaccccagtagtgagaaagggtcgctgttcctgcatcagcaccaaccaagggactatccacctacaatccttgaaagaccttaaacaatttgccccaagcccttcctgcgagaaaattgaaatcattgctacactgaagaatggagttcaaacatgtctaaacccagattcagcagatgtgaaggaactgattaaaaagtgggagaaacaggtcagccaaaagaaaaagcaaaagaatgggaaaaaacatcaaaaaaagaaagttctgaaagttcgaaaatctcaacgttctcgtcaaaagaagactacataagagaccacttcaccaataagtattctgtgttaaaaatgttctattttaattataccgctatcattccaaaggaggatggcatataatacaaaggcttattaatttgactagaaaatttaaaacattactctgaaattgtaactaaagttagaaagttgattttaagaatccaaacgttaagaattgttaaaggctatgattgtctttgttcttctaccacccaccagttgaatttcatcatgcttaaggccatgattttagcaatacccatgtctacacagatgttcacccaaccacatcccactcacaacagctgcctggaagagcagccctaggcttccacgtactgcagcctccagagagtatctgaggcacatgtcagcaagtcctaagcctgttagcatgctggtgagccaagcagtttgaaattgagctggacctcaccaagctgctgtggccatcaacctctgtatttgaatcagcctacaggcctcacacacaatgtgtctgagagattcatgctgattgttattgggtatcaccactggagatcaccagtgtgtggctttcagagcctcctttctggctttggaagccatgtgattccatcttgcccgctcaggctgaccactttatttctttttgttcccctttgcttcattcaagtcagctcttctccatcctaccacaatgcagtgcctttcttctctccagtgcacctgtcatatgctctgatttatctgagtcaactcctttctcatcttgtccccaacaccccacagaagtgctttcttctcccaattcatcctcactcagtccagcttagttcaagtcctgcctcttaaataaacctttttggacacacaaattatcttaaaactcctgtttcacttggttcagtaccacatgggtgaacactcaatggttaactaattcttgggtgtttatcctatctctccaaccagattgtcagctccttgagggcaagagccacagtatatttccctgtttcttccacagtgcctaataatactgtggaactaggttttaataattttttaattgatgttgttatgggcaggatggcaaccagaccattgtctcagagcaggtgctggctctttcctggctactccatgttggctagcctctggtaacctcttacttattatcttcaggacactcactacagggaccagggatgatgcaacatccttgtctttttatgacaggatgtttgctcagcttctccaacaataagaagcacgtggtaaaacacttgcggatattctggactgtttttaaaaaatatacagtttaccgaaaatcatataatcttacaatgaaaaggactttatagatcagccagtgaccaaccttttcccaaccatacaaaaattccttttcccgaaggaaaagggctttctcaataagcctcagctttctaagatctaacaagatagccaccgagatccttatcgaaactcattttaggcaaatatgagttttattgtccgtttacttgtttcagagtttgtattgtgattatcaattaccacaccatctcccatgaagaaagggaacggtgaagtactaagcgctagaggaagcagccaagtcggttagtggaagcatgattggtgcccagttagcctctgcaggatgtggaaacctccttccaggggaggttcagtgaattgtgtaggagaggttgtctgtggccagaatttaaacctatactcactttcccaaattgaatcactgctcacactgctgatgatttagagtgctgtccggtggagatcccacccgaacgtcttatctaatcatgaaactccctagttccttcatgtaacttccctgaaaaatctaagtgtttcataaatttgagagtctgtgacccacttaccttgcatctcacaggtagacagtatataactaacaaccaaagactacatattgtcactgacacacacgttataatcatttatcatatatatacatacatgcatacactctcaaagcaaataatttttcacttcaaaacagtattgacttgtataccttgtaatttgaaatattttctttgttaaaatagaatggtatcaataaatagaccattaatcag

SEQ ID NO: 5 - DNA, 698 bp, Homo sapiens
accccaccctaatcttgttatgcaaataggcttcccacttggcaggggccgtcttgtccactcgtttctgtaaacatgggtggcaaaaagagaagatggagctgccatttagaacatgcctaatcccagcttcatcttgctgagcaaaaatgaaggagcctggacccaactttgttactgtgagaaagggtcttcattcattcaagatggcatttgttaagcacctactgtgagtagatgatctcctgtcaaagacagttaacaaatcctcggaatattgcttcatgtacagttattggagatgagtaacttacattctcttaattgtaatggttccttggaaagtcatcgtggaaaatgaaggctggctcatacattttcccagacaggaatttggctgccaacagggaattctaaacaactaaaaactccagatgatgaatgcacaacataatgatggttaaattaaaaaaaaaaaagagcacgacaaaccttggaaatcaagaaagttctggaatgatgaagctgttcatgccaagaccgaaagtgctggcccagtatgagtccattcagttcatgccgtgacaattttcttggaactcctttttattgttagttctcacttgtttccatatttagtgaatgtacatttaattgcaaagctgtcattaataaaaattcttatagtacctcaaaaa

SEQ ID NO: 6 - DNA, 1925 bp, Homo sapiens
actccaggtcccctatcctgtcctctgcaacccaaacgtccaggaggatcatgacctgcggatcaggatttggtgggcgcgccttcagctgcatctcggcctgcgggccgcgccccggccgctgctgcatcaccgccgccccctaccgtggcatctcctgctaccgcggcctcaccgggggcttcggcagccacagcgtgtgcggaggctttcgggccggctcctgcggacgcagcttcggctaccgctccgggggcgtgtgcgggcccagtcccccatgcatcaccaccgtgtcggtcaacgagagcctcctcacgcccctcaacctggagatcgaccccaacgcgcagtgcgtgaagcaggaggagaaggagcagatcaagtccctcaacagcaggttcgcggccttcatcgacaaggtgcgcttcctggagcagcagaacaaactgctggagacaaagctgcagttctaccagaaccgcgagtgttgccagagcaacctggagcccctgtttgagggctacatcgagactctgcggcgggaggccgagtgcgtggaggccgacagcgggaggctggcctcagagcttaaccacgtgcaggaggtgctggagggctacaagaagaagtatgaggaggaggtttctctgagagcaacagctgagaacgagtttgtggctctgaagaaggatgtggactgcgcctacctccgcaagtcagacctggaggccaacgtggaggccctgatccaggagatcgacttcctgaggcggctgtatgaggaggagatccgcattctccagtcgcacatctcagacacctccgtggttgtcaagctggacaacagccgggacctgaacatggactgcatcattgccgagattaaggcacagtatgacgacattgtcacccgcagccgggccgaggccgagtcctggtaccgcagcaagtgtgaggagatgaaggccacggtgatcaggcacggggagaccctgcgccgcaccaaggaggagatcaatgagctgaaccgcatgatccaaaggctgacggccgaggtggagaatgccaagtgccagaactccaagctggaggccgcggtggctcagtctgagcagcagggtgaggcagccctcagtgatgcccgctgcaagctggccgagctggagggcgccctgcagaaggccaagcaggacatggcctgcctgatcagggagtaccaggaggtgatgaactccaagctgggcctggacatcgagatcgccacctacaggcgcctgctggagggcgaggagcagaggctatgtgaaggcattggggctgtgaatgtctgtgtcagcagctcccggggcggggtcgtgtgcggggacctctgcgtgtcaggctcccggccagtgactggcagtgtctgcagcgctccgtgcaacgggaacgtggcggtgagcaccggcctgtgtgcgccctgcggccaattgaacaccacctgcggagggggttcctgcggcgtgggctcctgtggtatcagctccctgggtgtggggtcttgcggcagcagctgccggaaatgttaggcaccccaactcaagtcccaggccccaggcatctttgcctgccctgccttgcttggcccagtcagtcaggcgcctggagaagtgctcagctacttctcctgcactttgaaagacccctcccactcctggcctcacatttctctgtgtgatcccccacttctgggctctgccaccccacagtgggaaaggccaccctagaaagaagtccgctggcacccataggaaggggcctcaggagcaggaagggccaggaccagaaccttgcccacggcaactgccttcctgcctctccccttcctcctctgctcttgatctgtgtttcaataaattaatgtagccaaaaaaaaaaaaaaaaaa

SEQ ID NO: 7 - DNA, 3313 bp, Homo sapiens
aagagtaaaatgtctctttatctggaacttacatgatgatttttccaacaaaataatttccaaatagatcacatagaaaatgtctcttttaatatactttacagtagtaaaactataacatctcaattgttttttttaaataaatttgaataatggtttaggtcatttgataaatatcaccttgtaactaataagatgaaacaaggctaaataggaccaattaagcacgtgatttaaatatcgatatgtagtgagtaaaagagtaatccaactaccagtagaagattactacatttagtactataacaagtataacactgttcctaaaaaaaagtgctttcttatgtttaagatttattttaatgtcaaacacaaataattagatctttaaatgaacaatttgggagttaaatccattgcttctgatttttatagattttatggtctaggaaatctatactgtctgatttgatcccatttaactgtaagatttttacacatgttgcactctactagctggcaggaaaattattttaatcgactgaatgaaagtattatttcatgtaaaagtttattatatcaaggaaatgatttaggtcagaagctagaatctatataaagtcagcttttgaaaataaagacagacaaatctttttttacattattataaaagagctaagttgcaaacaactatccttgagaccagaccatttttttttaagctgaaattttctaataattgcaatggcaaaacaccatttgcaatttcttccctcccacctcccaggtggttcaacaattccacttccaaacagcatttcccatcagtttttaaaagctacttacaaagtgttattctactaccacttttaaatacatcaagcacttccaaatatctagaaagactagatatttcatataacttgtccaccacatacacatcactgttaaataaaattgcacacacataacaatggttatcatctgaggtatcttctaaatgtggccattttggccttgaatcattccctcctcccttccttctctgccttcaatccagtggacaagtacaggcacatgtaatgcttagagatggtcgaacaaattcctatgcaaaagtctttacagaagacaagttttcctatgaattttaacacaaagcgtacaaaatatgctaattttactactttgtcatacactggcaacctctttaacaactagagactagatgttgaaaaattaggactatttgtccattatatatactatatacagagcaaaacaaaatgcacaaaacgtatagaaaaatggtgtctgaaaatgtccaagtatgaacacactagtatattacctcttgcaatttcttccctcctacctcctctaaaccattgaacaagtatacacattactatactgctcacaaaggtggcttcacaattcaatttccaaaagcatttcctatgaattttagcaaaaagatatttacaaagtggtattttactacctatacatttaacatacatcgggcacttctaaacatctagatagactagatgtttcaagtaaggagttaatttgtctactatgtatacagcagtcttgaataaactgcaaacatgtaacaacagttataatttgaaagagtcttccaaatgtgaacattctggcctagaacccttcccatcgccatcaacccagaagacatcaaattttcagaagacaatctttcctaggacttgtaaaacaaaatgtacaaaatatattagtttactaactctacttttgtcatacactggcaacctctttaacatccagaaagactagatgttgtcaattaggactcgtctgtcctttatgtacattatatacacagataagtaaaacaaaatgcacagacatacatcttgcctcgctgtaaacaggatggcatagagctctctgcacctccccctcctctctcctcccctgaaccactgcacaaacacaatgagtattactcaacaggtgatttgaccattccccccaaaaaatatttcctatgaattgtaacaaaaaggtatttacaaaatgtgattttgctacctctaattttaacatatcaggcacttcagaacatctaaaaagaagagacatttcaaaaaagcttagcattgtcaactatatacacagtagtgaggaataaaatgcacacaaaacaatggatagaatatgaaaatgtcttctaaatatgaccagtctagcatagaaccttcttctcttccttctcaggtcttccagctccatgtcatctaacccacttaacaaacgtgaacgtatcgcttccagaggccgtcttaacaattccatttccaaaagtcatctccagaagacatgtattttctacgatttcttttaaacaaatgagaatttacaagatgtgtaactttctaactcttttatcataactcgacaacctctttccatctagaagggctagatgtgacaaatgttttctattaaaaggttggggtggagttgagagcagctttttcatattatatacacaggccttccataaacggccagtaaatcttcccagagggtggtgggcatttccaactggccaaacgtggcctgtcattctaccatttctctcttccgacagcaaagtctggtagaatgaagaccaaccgcccgatggccgctaaccgttccacccgtcgttgttcgggacttcgctcacctttcaggccccttaaggcctttgtccgttgtcgtcaggactaggtaggtctcgcccaatggcgacagagtggtcacccgggaaccggatctgcgcggctccgtggccgaaagaggcggccaagcctgcttgcgtccctaggccgccttccgggccgtccacgccttaatggcctccgccgcgcggcgttcgagcggccgccatacttcccggcccaccacgcccggcgccgcccaaaggcgctgcgtcctggcggctctgcgggggtttcgtcgaggcccagcaggcttgggtcgggagacccgggtgccggcgggggccgggctgggagacgccacggccgccatcagtcaccgaggtggggtgggaaagagaggttcgctgcggcttcaaggtctgagcacagccagtgggcagccacagcagaggcctccggtgtctgcagggcagagggctcggcctgtcccgaggccccccagttcatccgccggcccggggccagagggccctgaaggcgcgggctgcgttctgcgtctctccgcgatctctgccggaccggaactaagaccagaccattttcttctggagtaa

SEQ ID NO: 8 - DNA, 1860 bp, Homo sapiens
ctggttcgcaaagaagctgacttcagagggggaaactttcttcttttaggaggcggttagccctgttccacgaacccaggagaactgctggccagattaattagacattgctatgggagacgtgtaaacacactacttatcattgatgcatatataaaaccattttattttcgctattatttcagaggaagcgcctctgatttgtttcttttttccctttttgctctttctggctgtgtggtttggagaaagcacagttggagtagccggttgctaaataagtcccgagcgcgagcggagacgatgcagcggagactggttcagcagtggagcgtcgcggtgttcctgctgagctacgcggtgccctcctgcgggcgctcggtggagggtctcagccgccgcctcaaaagagctgtgtctgaacatcagctcctccatgacaaggggaagtccatccaagatttacggcgacgattcttccttcaccatctgatcgcagaaatccacacagctgaaatcagagctacctcggaggtgtcccctaactccaagccctctcccaacacaaagaaccaccccgtccgatttgggtctgatgatgagggcagatacctaactcaggaaactaacaaggtggagacgtacaaagagcagccgctcaagacacctgggaagaaaaagaaaggcaagcccgggaaacgcaaggagcaggaaaagaaaaaacggcgaactcgctctgcctggttagactctggagtgactgggagtgggctagaaggggaccacctgtctgacacctccacaacgtcgctggagctcgattcacggtaacaggcttctctggcccgtagcctcagcggggtgctctcagctgggttttggagcctcccttctgccttggcttggacaaacctagaattttctccctttatgtatctctatcgattgtgtagcaattgacagagaataactcagaatattgtctgccttaaagcagtacccccctaccacacacacccctgtcctccagcaccatagagaggcgctagagcccattcctctttctccaccgtcacccaacatcaatcctttaccactctaccaaataatttcatattcaagcttcagaagctagtgaccatcttcataatttgctggagaagtgtgtttcttccccttactctcacacctgggcaaactttcttcagtgtttttcatttcttacgttctttcacttcaagggagaatatagaagcatttgatattatctacaaacactgcagaacagcatcatgtcataaacgattctgagccattcacactttttatttaattaaatgtatttaattaaatctcaaatttattttaatgtaaagaacttaaattatgttttaaacacatgccttaaatttgtttaattaaatttaactctggtttctaccagctcatacaaaataaatggtttctgaaaatgtttaagtattaacttacaaggatataggtttttctcatgtatctttttgttcattggcaagatgaaataatttttctagggtaatgccgtaggaaaaataaaacttcacatttatgtggcttgtttatccttagctcacagattgaggtaataatgacactcctagactttgggatcaaataacttagggccaagtcttgggtctgaatttatttaagttcacaacctagggcaagttactctgcctttctaagactcacttacatcttctgtgaaatataattgtaccaacctcatagagtttggtgtcaactaaatgagattatatgtggactaaatatctgtcatatagtaaacactcaataaattgcaacatattaaaaaaa

SEQ ID NO: 9 - DNA, 2276 bp, Homo sapiens
aagcccagcagccccggggcggatggctccggccgcctggctccgcagcgcggccgcgcgcgccctcctgcccccgatgctgctgctgctgctccagccgccgccgctgctggcccgggctctgccgccggacgcccaccacctccatgccgagaggagggggccacagccctggcatgcagccctgcccagtagcccggcacctgcccctgccacgcaggaagccccccggcctgccagcagcctcaggcctccccgctgtggcgtgcccgacccatctgatgggctgagtgcccgcaaccgacagaagaggttcgtgctttctggcgggcgctgggagaagacggacctcacctacaggatccttcggttcccatggcagttggtgcaggagcaggtgcggcagacgatggcagaggccctaaaggtatggagcgatgtgacgccactcacctttactgaggtgcacgagggccgtgctgacatcatgatcgacttcgccaggtactggcatggggacgacctgccgtttgatgggcctgggggcatcctggcccatgccttcttccccaagactcaccgagaaggggatgtccacttcgactatgatgagacctggactatcggggatgaccagggcacagacctgctgcaggtggcagcccatgaatttggccacgtgctggggctgcagcacacaacagcagccaaggccctgatgtccgccttctacacctttcgctacccactgagtctcagcccagatgactgcaggggcgttcaacacctatatggccagccctggcccactgtcacctccaggaccccagccctgggcccccaggctgggatagacaccaatgagattgcaccgctggagccagacgccccgccagatgcctgtgaggcctcctttgacgcggtctccaccatccgaggcgagctctttttcttcaaagcgggctttgtgtggcgcctccgtgggggccagctgcagcccggctacccagcattggcctctcgccactggcagggactgcccagccctgtggacgctgccttcgaggatgcccagggccacatttggttcttccaaggtgctcagtactgggtgtacgacggtgaaaagccagtcctgggccccgcacccctcaccgagctgggcctggtgaggttcccggtccatgctgccttggtctggggtcccgagaagaacaagatctacttcttccgaggcagggactactggcgtttccaccccagcacccggcgtgtagacagtcccgtgccccgcagggccactgactggagaggggtgccctctgagatcgacgctgccttccaggatgctgatggctatgcctacttcctgcgcggccgcctctactggaagtttgaccctgtgaaggtgaaggctctggaaggcttcccccgtctcgtgggtcctgacttctttggctgtgccgagcctgccaacactttcctctgaccatggcttggatgccctcaggggtgctgacccctgccaggccacgaatatcaggctagagacccatggccatctttgtggctgtgggcaccaggcatgggactgagcccatgtctcctcagggggatggggtggggtacaaccaccatgacaactgccgggagggccacgcaggtcgtggtcacctgccagcgactgtctcagactgggcagggaggctttggcatgacttaagaggaagggcagtcttgggcccgctatgcaggtcctggcaaacctggctgccctgtctccatccctgtccctcagggtagcaccatggcaggactgggggaactggagtgtccttgctgtatccctgttgtgaggttccttccaggggctggcactgaagcaagggtgctggggccccatggccttcagccctggctgagcaactgggctgtagggcagggccacttcctgaggtcaggtcttggtaggtgcctgcatctgtctgccttctggctgacaatcctggaaatctgttctccagaatccaggccaaaaagttcacagtcaaatggggaggggtattcttcatgcaggagaccccaggccctggaggctgcaacatacctcaatcctgtcccaggccggatcctcctgaagcccttttcgcagcactgctatcctccaaagccattgtaaatgtgtgtacagtgtgtataaaccttcttcttctttttttttttttaaactgaggattgtcattaaacacagttgttttctaaaaaaaaaaaaaaaa

SEQ ID NO: 10 - DNA, 450 bp, Homo sapiens
gtccaaacacacacatctcactcatccttctactcgtgacgcttcccagctctggctttttgaaagcaaagatgagcaacactcaagctgagaggtccataataggcatgatcgacatgtttcacaaatacaccagacgtgatgacaagattgagaagccaagcctgctgacgatgatgaaggagaacttccccaacttccttagtgcctgtgacaaaaagggcacaaattacctcgccgatgtctttgagaaaaaggacaagaatgaggataagaagattgatttttctgagtttctgtccttgctgggagacatagccacagactaccacaagcagagccatggagcagcgccctgttccgggggcagccagtgacccagccccaccaatgggcctccagagaccccaggaacaataaaatgtcttctcccaccagaaaaaaaaaaaa

SEQ ID NO: 11 - DNA, 1307 bp, Homo sapiens
cctgagtcccgggaggaaagtgctcgcccattcctgacctgtgacacgctcactgcgaaggcaggttattagaagagtcccatgaaaggtggctccacggtcccagcgacatgcaggggctcctcttctccactcttctgcttgctggcctggcacagttctgctgcagggtacagggcactggaccattagatacaacacctgaaggaaggcctggagaagtgtcagatgcacctcagcgtaaacagttttgtcactggccctgcaaatgccctcagcagaagccccgttgccctcctggagtgagcctggtgagagatggctgtggatgctgtaaaatctgtgccaagcaaccaggggaaatctgcaatgaagctgacctctgtgacccacacaaagggctgtattgtgactactcagtagacaggcctaggtacgagactggagtgtgtgcataccttgtagctgttgggtgcgagttcaaccaggtacattatcataatggccaagtgtttcagcccaaccccttgttcagctgcctctgtgtgagtggggccattggatgcacacctctgttcataccaaagctggctggcagtcactgctctggagctaaaggtggaaagaagtctgatcagtcaaactgtagcctggaaccattactacagcagctttcaacaagctacaaaacaatgccagcttatagaaatctcccacttatttggaaaaaaaaatgtcttgtgcaagcaacaaaatggactccctgctccagaacatgtgggatgggaatatctaacagggtgaccaatgaaaacagcaactgtgaaatgagaaaagagaaaagactgtgttacattcagccttgcgacagcaatatattaaagacaataaagattcccaaaggaaaaacatgccaacctactttccaactctccaaagctgaaaaatttgtcttttctggatgctcaagtactcagagttacaaacccactttttgtggaatatgcttggataagagatgctgtatccctaataagtctaaaatgattactattcaatttgattgcccaaatgaggggtcatttaaatggaagatgctgtggattacatcttgtgtgtgtcagagaaactgcagagaacctggagatatattttctgagctcaagattctgtaaaaccaagcaaatgggggaaaagttagtcaatcctgtcatataataaaaaaattagtgagtaaaaaaaaaaaaaaaaaaaaaaaaaaaaaaaaaaaaaaaaaaaaaaaaaaagaaaaaaaaaaaaaaaaaaaa

SEQ ID NO: 12 - DNA, 1184 bp, Homo sapiens
gggggagacattcctcaattgcttagacatattctgagcctacagcagaggaacctccagtctcagcaccatgaatcaaactgccattctgatttgctgccttatctttctgactctaagtggcattcaaggagtacctctctctagaactgtacgctgtacctgcatcagcattagtaatcaacctgttaatccaaggtctttagaaaaacttgaaattattcctgcaagccaattttgtccacgtgttgagatcattgctacaatgaaaaagaagggtgagaagagatgtctgaatccagaatcgaaggccatcaagaatttactgaaagcagttagcaaggaaaggtctaaaagatctccttaaaaccagaggggagcaaaatcgatgcagtgcttccaaggatggaccacacagaggctgcctctcccatcacttccctacatggagtatatgtcaagccataattgttcttagtttgcagttacactaaaaggtgaccaatgatggtcaccaaatcagctgctactactcctgtaggaaggttaatgttcatcatcctaagctattcagtaataactctaccctggcactataatgtaagctctactgaggtgctatgttcttagtggatgttctgaccctgcttcaaatatttccctcacctttcccatcttccaagggtactaaggaatctttctgctttggggtttatcagaattctcagaatctcaaataactaaaaggtatgcaatcaaatctgctttttaaagaatgctctttacttcatggacttccactgccatcctcccaaggggcccaaattctttcagtggctacctacatacaattccaaacacatacaggaaggtagaaatatctgaaaatgtatgtgtaagtattcttatttaatgaaagactgtacaaagtagaagtcttagatgtatatatttcctatattgttttcagtgtacatggaataacatgtaattaagtactatgtatcaatgagtaacaggaaaattttaaaaatacagatagatatatgctctgcatgttacataagataaatgtgctgaatggttttcaaaataaaaatgaggtactctcctggaaatattaagaaagactatctaaatgttgaaagatcaaaaggttaataaagtaattataactaaaaaaa

SEQ ID NO: 13 - DNA, 817 bp, Homo sapiens
agtcctgcgtccgggccccgaggcgcagcagggcaccaggtggagcaccagctacgcgtggcgcagcgcagcgtccctagcaccgagcctcccgcagccgccgagatgctgcgaacagagagctgccgccccaggtcgcccgccggacaggtggccgcggcgtccccgctcctgctgctgctgctgctgctcgcctggtgcgcgggcgcctgccgaggtgctccaatattacctcaaggattacagcctgaacaacagctacagttgtggaatgagatagatgatacttgttcgtcttttctgtccattgattctcagcctcaggcatccaacgcactggaggagctttgctttatgattatgggaatgctaccaaagcctcaggaacaagatgaaaaagataatactaaaaggttcttatttcattattcgaagacacagaagttgggcaagtcaaatgttgtgtcgtcagttgtgcatccgttgctgcagctcgttcctcacctgcatgagagaagaatgaagagattcagagtggacgaagaattccaaagtccctttgcaagtcaaagtcgaggatattttttattcaggccacggaatggaagaaggtcagcagggttcatttaaaatggatgccagctaattttccacagagcaatgctatggaatacaaaatgtactgacattttgttttcttctgaaaaaaatccttgctaaatgtactctgttgaaaatccctgtgttgtcaatgttctcagttgtaacaatgttgtaaatgttcaatttgttgaaaattaaaaaatctaaaaataaa

SEQ ID NO: 14 - DNA, 2431 bp, Homo sapiens
ctccaggctgtggaacctttgttctttcactctttgcaataaatcttgctgctgctcactctttgggtccacactgcctttatgagctgtaacactcactgggaatgtctgcagcttcactcctgaagccagcgagaccacgaacccaccaggaggaacaaacaactccagacgcgcagccttaagagctgtaacactcaccgcgaaggtctgcagcttcactcctgagccagccagaccacgaacccaccagaaggaagaaactccaaacacatccgaacatcagaaggagcaaactcctgacacgccacctttaagaaccgtgacactcaacgctagggtccgcggcttcattcttgaagtcagtgagaccaagaacccaccaattccggacacgctaattgttgtagatcatcacttcaaggtgcccatatctttctagtggaaaaattattctggcctccgctgcatacaaatcaggcaaccagaattctacatatataaggcaaagtaacatcctagacatggctttagagatccacatgtcagaccccatgtgcctcatcgagaactttaatgagcagctgaaggttaatcaggaagctttggagatcctgtctgccattacgcaacctgtagttgtggtagcgattgtgggcctctatcgcactggcaaatcctacctgatgaacaagctggctgggaagaacaagggcttctctgttgcatctacggtgcagtctcacaccaagggaatttggatatggtgtgtgcctcatcccaactggccaaatcacacattagttctgcttgacaccgagggcctgggagatgtagagaaggctgacaacaagaatgatatccagatctttgcactggcactcttactgagcagcacctttgtgtacaatactgtgaacaaaattgatcagggtgctatcgacctactgcacaatgtgacagaactgacagatctgctcaaggcaagaaactcacccgaccttgacagggttgaagatcctgctgactctgcgagcttcttcccagacttagtgtggactctgagagatttctgcttaggcctggaaatagatgggcaacttgtcacaccagatgaatacctggagaattccctaaggccaaagcaaggtagtgatcaaagagttcaaaatttcaatttgccccgtctgtgtatacagaagttctttccaaaaaagaaatgctttatctttgacttacctgctcaccaaaaaaagcttgcccaacttgaaacactgcctgatgatgagctagagcctgaatttgtgcaacaagtgacagaattctgttcctacatctttagccattctatgaccaagactcttccaggtggcatcatggtcaatggatctcgtctaaagaacctggtgctgacctatgtcaatgccatcagcagtggggatctgccttgcatagagaatgcagtcctggccttggctcagagagagaactcagctgcagtgcaaaaggccattgcccactatgaccagcaaatgggccagaaagtgcagctgcccatggaaaccctccaggagctgctggacctgcacaggaccagtgagagggaggccattgaagtcttcatgaaaaactctttcaaggatgtagaccaaagtttccagaaagaattggagactctactagatgcaaaacagaatgacatttgtaaacggaacctggaagcatcctcggattattgctcggctttacttaaggatatttttggtcctctagaagaagcagtgaagcagggaatttattctaagccaggaggccataatctcttcattcagaaaacagaagaactgaaggcaaagtactatcgggagcctcggaaaggaatacaggctgaagaagttctgcagaaatatttaaagtccaaggagtctgtgagtcatgcaatattacagactgaccaggctctcacagagacggaaaaaaagaagaaagaggcacaagtgaaagcagaagctgaaaaggctgaagcgcaaaggttggcggcgattcaaaggcagaacgagcaaatgatgcaggagagggagagactccatcaggaacaagtgagacaaatggagatagccaaacaaaattggctggcagagcaacagaaaatgcaggaacaacagatgcaggaacaggctgcacagctcagcacaacattccaagctcaaaatagaagccttctcagtgagctccagcacgcccagaggactgttaataacgatgatccatgtgttttactctaaagtgctaaatatgggagtttcctttttttactctttgtcactgatgacacaacagaaaagaaactgtagaccttgggacaatcaacatttaaataaactttataattattttttcaaactttaaaaaaaaaaaaaaaaaaaaaaaa

SEQ ID NO: 15 - DNA, 5698 bp, Homo sapiens
aggttcaagtggagctctcctaaccgacgcgcgtctgtggagaagcggcttggtcgggggtggtctcgtggggtcctgcctgtttagtcgctttcagggttcttgagccccttcacgaccgtcaccatggaagtgtcaccattgcagcctgtaaatgaaaatatgcaagtcaacaaaataaagaaaaatgaagatgctaagaaaagactgtctgttgaaagaatctatcaaaagaaaacacaattggaacatattttgctccgcccagacacctacattggttctgtggaattagtgacccagcaaatgtgggtttacgatgaagatgttggcattaactatagggaagtcacttttgttcctggtttgtacaaaatctttgatgagattctagttaatgctgcggacaacaaacaaagggacccaaaaatgtcttgtattagagtcacaattgatccggaaaacaatttaattagtatatggaataatggaaaaggtattcctgttgttgaacacaaagttgaaaagatgtatgtcccagctctcatatttggacagctcctaacttctagtaactatgatgatgatgaaaagaaagtgacaggtggtcgaaatggctatggagccaaattgtgtaacatattcagtaccaaatttactgtggaaacagccagtagagaatacaagaaaatgttcaaacagacatggatggataatatgggaagagctggtgagatggaactcaagcccttcaatggagaagattatacatgtatcacctttcagcctgatttgtctaagtttaaaatgcaaagcctggacaaagatattgttgcactaatggtcagaagagcatatgatattgctggatccaccaaagatgtcaaagtctttcttaatggaaataaactgccagtaaaaggatttcgtagttatgtggacatgtatttgaaggacaagttggatgaaactggtaactccttgaaagtaatacatgaacaagtaaaccacaggtgggaagtgtgtttaactatgagtgaaaaaggctttcagcaaattagctttgtcaacagcattgctacatccaagggtggcagacatgttgattatgtagctgatcagattgtgactaaacttgttgatgttgtgaagaagaagaacaagggtggtgttgcagtaaaagcacatcaggtgaaaaatcacatgtggatttttgtaaatgccttaattgaaaacccaacctttgactctcagacaaaagaaaacatgactttacaacccaagagctttggatcaacatgccaattgagtgaaaaatttatcaaagctgccattggctgtggtattgtagaaagcatactaaactgggtgaagtttaaggcccaagtccagttaaacaagaagtgttcagctgtaaaacataatagaatcaagggaattcccaaactcgatgatgccaatgatgcagggggccgaaactccactgagtgtacgcttatcctgactgagggagattcagccaaaactttggctgtttcaggccttggtgtggttgggagagacaaatatggggttttccctcttagaggaaaaatactcaatgttcgagaagcttctcataagcagatcatggaaaatgctgagattaacaatatcatcaagattgtgggtcttcagtacaagaaaaactatgaagatgaagattcattgaagacgcttcgttatgggaagataatgattatgacagatcaggaccaagatggttcccacatcaaaggcttgctgattaattttatccatcacaactggccctctcttctgcgacatcgttttctggaggaatttatcactcccattgtaaaggtatctaaaaacaagcaagaaatggcattttacagccttcctgaatttgaagagtggaagagttctactccaaatcataaaaaatggaaagtcaaatattacaaaggtttgggcaccagcacatcaaaggaagctaaagaatactttgcagatatgaaaagacatcgtatccagttcaaatattctggtcctgaagatgatgctgctatcagcctggcctttagcaaaaaacagatagatgatcgaaaggaatggttaactaatttcatggaggatagaagacaacgaaagttacttgggcttcctgaggattacttgtatggacaaactaccacatatctgacatataatgacttcatcaacaaggaacttatcttgttctcaaattctgataacgagagatctatcccttctatggtggatggtttgaaaccaggtcagagaaaggttttgtttacttgcttcaaacggaatgacaagcgagaagtaaaggttgcccaattagctggatcagtggctgaaatgtcttcttatcatcatggtgagatgtcactaatgatgaccattatcaatttggctcagaattttgtgggtagcaataatctaaacctcttgcagcccattggtcagtttggtaccaggctacatggtggcaaggattctgctagtccacgatacatctttacaatgctcagctctttggctcgattgttatttccaccaaaagatgatcacacgttgaagtttttatatgatgacaaccagcgtgttgagcctgaatggtacattcctattattcccatggtgctgataaatggtgctgaaggaatcggtactgggtggtcctgcaaaatccccaactttgatgtgcgtgaaattgtaaataacatcaggcgtttgatggatggagaagaacctttgccaatgcttccaagttacaagaacttcaagggtactattgaagaactggctccaaatcaatatgtgattagtggtgaagtagctattcttaattctacaaccattgaaatctcagagcttcccgtcagaacatggacccagacatacaaagaacaagttctagaacccatgttgaatggcaccgagaagacacctcctctcataacagactatagggaataccatacagataccactgtgaaatttgttgtgaagatgactgaagaaaaactggcagaggcagagagagttggactacacaaagtcttcaaactccaaactagtctcacatgcaactctatggtgctttttgaccacgtaggctgtttaaagaaatatgacacggtgttggatattctaagagacttttttgaactcagacttaaatattatggattaagaaaagaatggctcctaggaatgcttggtgctgaatctgctaaactgaataatcaggctcgctttatcttagagaaaatagatggcaaaataatcattgaaaataagcctaagaaagaattaattaaagttctgattcagaggggatatgattcggatcctgtgaaggcctggaaagaagcccagcaaaaggttccagatgaagaagaaaatgaagagagtgacaacgaaaaggaaactgaaaagagtgactccgtaacagattctggaccaaccttcaactatcttcttgatatgcccctttggtatttaaccaaggaaaagaaagatgaactctgcaggctaagaaatgaaaaagaacaagagctggacacattaaaaagaaagagtccatcagatttgtggaaagaagacttggctacatttattgaagaattggaggctgttgaagccaaggaaaaacaagatgaacaagtcggacttcctgggaaaggggggaaggccaaggggaaaaaaacacaaatggctgaagttttgccttctccgcgtggtcaaagagtcattccacgaataaccatagaaatgaaagcagaggcagaaaagaaaaataaaaagaaaattaagaatgaaaatactgaaggaagccctcaagaagatggtgtggaactagaaggcctaaaacaaagattagaaaagaaacagaaaagagaaccaggtacaaagacaaagaaacaaactacattggcatttaagccaatcaaaaaaggaaagaagagaaatccctggtctgattcagaatcagataggagcagtgacgaaagtaattttgatgtccctccacgagaaacagagccacggagagcagcaacaaaaacaaaattcacaatggatttggattcagatgaagatttctcagattttgatgaaaaaactgatgatgaagattttgtcccatcagatgctagtccacctaagaccaaaacttccccaaaacttagtaacaaagaactgaaaccacagaaaagtgtcgtgtcagaccttgaagctgatgatgttaagggcagtgtaccactgtcttcaagccctcctgctacacatttcccagatgaaactgaaattacaaacccagttcctaaaaagaatgtgacagtgaagaagacagcagcaaaaagtcagtcttccacctccactaccggtgccaaaaaaagggctgccccaaaaggaactaaaagggatccagctttgaattctggtgtctctcaaaagcctgatcctgccaaaaccaagaatcgccgcaaaaggaagccatccacttctgatgattctgactctaattttgagaaaattgtttcgaaagcagtcacaagcaagaaatccaagggggagagtgatgacttccatatggactttgactcagctgtggctcctcgggcaaaatctgtacgggcaaagaaacctataaagtacctggaagagtcagatgaagatgatctgttttaaaatgtgaggcgattattttaagtaattatcttaccaagcccaagactggttttaaagttacctgaagctcttaacttcctcccctctgaatttagtttggggaaggtgtttttagtacaagacatcaaagtgaagtaaagcccaagtgttctttagctttttataatactgtctaaatagtgaccatctcatgggcattgttttcttctctgctttgtctgtgttttgagtctgctttcttttgtctttaaaacctgatttttaagttcttctgaactgtagaaatagctatctgatcacttcagcgtaaagcagtgtgtttattaaccatccactaagctaaaactagagcagtttgatttaaaagtgtcactcttcctccttttctactttcagtagatatgagatagagcataattatctgttttatcttagttttatacataatttaccatcagatagaactttatggttctagtacagatactctactacactcagcctcttatgtgccaagtttttctttaagcaatgagaaattgctcatgttcttcatcttctcaaatcatcagaggccaaagaaaaacactttggctgtgtctataacttgacacagtcaatagaatgaagaaaattagagtagttatgtgattatttcagctcttgacctgtcccctctggctgcctctgagtctgaatctcccaaagagagaaaccaatttctaagaggactggattgcagaagactcggggacaacatttgatccaagatcttaaatgttatattgataaccatgctcagcaatgagctattagattcattttgggaaatctccataatttcaatttgtaaactttgttaagacctgtctacattgttatatgtgtgtgacttgagtaatgttatcaacgtttttgtaaatatttactatgtttttctattagctaaattccaacaattttgtactttaataaa

SEQ ID NO: 16 - DNA, 1736 bp, Homo sapiens
ccttcattccacagacacacacagcctctctgcccacctctgcttcctctaggaacacaggagttccagatcacatcgagttcaccatgaattcactcagtgaagccaacaccaagttcatgttcgatctgttccaacagttcagaaaatcaaaagagaacaacatcttctattcccctatcagcatcacatcagcattagggatggtcctcttaggagccaaagacaacactgcacaacaaattagcaaggttcttcactttgatcaagtcacagagaacaccacagaaaaagctgcaacatatcatgttgataggtcaggaaatgttcatcaccagtttcaaaagcttctgactgaattcaacaaatccactgatgcatatgagctgaagatcgccaacaagctcttcggagaaaagacgtatcaatttttacaggaatatttagatgccatcaagaaattttaccagaccagtgtggaatctactgattttgcaaatgctccagaagaaagtcgaaagaagattaactcctgggtggaaagtcaaacgaatgaaaaaattaaaaacctatttcctgatgggactattggcaatgatacgacactggttcttgtgaacgcaatctatttcaaagggcagtgggagaataaatttaaaaaagaaaacactaaagaggaaaaattttggccaaacaagaatacatacaaatctgtacagatgatgaggcaatacaattcctttaattttgccttgctggaggatgtacaggccaaggtcctggaaataccatacaaaggcaaagatctaagcatgattgtgctgctgccaaatgaaatcgatggtctgcagaagcttgaagagaaactcactgctgagaaattgatggaatggacaagtttgcagaatatgagagagacatgtgtcgatttacacttacctcggttcaaaatggaagagagctatgacctcaaggacacgttgagaaccatgggaatggtgaatatcttcaatggggatgcagacctctcaggcatgacctggagccacggtctctcagtatctaaagtcctacacaaggcctttgtggaggtcactgaggagggagtggaagctgcagctgccaccgctgtagtagtagtcgaattatcatctccttcaactaatgaagagttctgttgtaatcaccctttcctattcttcataaggcaaaataagaccaacagcatcctcttctatggcagattctcatccccatagatgcaattagtctgtcactccatttagaaaatgttcacctagaggtgttctggtaaactgattgctggcaacaacagattctcttggctcatatttcttttctatctcatcttgatgatgatagtcatcatcaagaatttaatgattaaaatagcatgcctttctctctttctcttaataagcccacatataaatgtacttttccttccagaaaaatttcccttgaggaaaaatgtccaagataagatgaatcatttaataccgtgtcttctaaatttgaaatataattctgtttctgacctgttttaaatgaaccaaaccaaatcatactttctcttcaaatttagcaacctagaaacacacatttctttgaatttaggtgatacctaaatccttcttatgtttctaaattttgtgattctataaaacacatcatcaataaaataatgacataaaatcaaaaaaaaaaaaaaaaaa

SEQ ID NO: 17 - DNA, 853 bp, Homo sapiens
cctgggccctcctgctccttgcagccatgctcctgggcaacccagcccctgcctccgcatctgcgtggtgaaggccattggcctcatcggtggatctgcgtttcctcgggcccacactgtctaggattgtgcggggctggtgagagaacaagatctcttccgtgttcaaggcagacttcctgccccctgcaccctgctctctcccgggccttgaggtcagtgtgagccccaagggcaagaacacttctggaagggagagtggatttggctgggcctctggatggaaggtctggtcttctctcgtctgagccctgagtactacgacccggcaagagcccacctgcgtgatggggagaaatcctgcccgtgcgggcaggagggcccccagggtgacctgttgaccaaaacacaggagctgggccgtgactacaggacctgtctgacgatagtccaaaaactgaagaagatggtggataagcccacccagagaagtgtttccaatgctgcgacccgggtgtgtaggacggggaggtcacgatggcgcgacgtctgcagaaatttcatgaggaggtatcagtctagagttatccaaggcctcgtggccggagaaactgcccagcagatctgtgaggacctcaggttgtgtataccttctacaggtcccctctgagccctctcaccttgtcctgtggaagaagcacaggctcctgtcctcagatcccgggaacgtcagcaacctctgccggctcctcgcttcctcgatccagaatccactctccagtctccctcccctgactccctctgctgtcctcccctctcaggggaataaagtgtcaagcaagattttagccgc

SEQ ID NO: 18 - DNA, 785 bp, Homo sapiens
agcggaggggtgtgtccaccgagcacttggattcagcttcttcatttccaacatggaagaaacttacaccgactccctggaccctgagaagctattgcaatgcccctatgacaaaaaccatcaaatcagggcttgcaggtttccttatcatcttatcaagtgcagaaagaatcatcctgatgttgcaagcaaattggctacttgtcccttcaatgctcgccaccaggttcctcgagctgaaattagtcatcatatctcaagctgtgatgacagaagttgtattgagcaagatgttgtcaaccaaaccaggagccttagacaagagactctggctgagagcacttggcagtgccctccttgcgatgaagactgggataaagatttgtgggagcagaccagcaccccatttgcctggggcacaactcactactctgacaacaacagccctgcgagcaacatagttacagaacataagaataacctggcttcaggcatgcgagttcccaaatctctgccgtatgttctgccatggaaaaacaatggaaatgcacagtaactgaatacctatctcatcaaatgccagaccctagaagactgttgcttcttcttctaccagtgggttctcattttcctcctaatctaattatagaatagtaaactccctgtgactttccaaactgacaagcacacttttttcctccccccttgaatcctcatttaatgcaagaaccctcatactcagaagcttccaaataaacctttgatacagaaaaaaaaaaaaaaaa

SEQ ID NO: 19 - DNA, 623 bp, Homo sapiens
aggccaagctggactgcataaagattggtatggccttagctcttagccaaacaccttcctgacaccatgagggccagcagcttcttgatcgtggtggtgttcctcatcgctgggacgctggttctagaggcagctgtcacgggagttcctgttaaaggtcaagacactgtcaaaggccgtgttccattcaatggacaagatcccgttaaaggacaagtttcagttaaaggtcaagataaagtcaaagcgcaagagccagtcaaaggtccagtctccactaagcctggctcctgccccattatcttgatccggtgcgccatgttgaatccccctaaccgctgcttgaaagatactgactgcccaggaatcaagaagtgctgtgaaggctcttgcgggatggcctgtttcgttccccagtgagagggagccggtccttgctgcacctgtgccgtccccagagctacaggccccatctggtcctaagtccctgctgcccttccccttcccacactgtccattcttcctcccattcaggatgcccacggctggagctgcctctctcatccactttccaataaagagttccttctgctccaaaaaaaaaaaaaaaaaaaaaaaaaaaa

SEQ ID NO: 20 - DNA, 4279 bp, Homo sapiens
atctcactcatccttctactcgtgacacttcccagttctggctttttgaaagcaaagatgagcaacactcaagctgagaggtccataataggcatgatcgacatgtttcacaaatacaccggacgtgatggcaagattgagaagccaagcctgctgacgatgatgaaggagaacttccccaatttcctcagtgcctgtgacaaaaagggcatacattacctcgccactgtctttgagaaaaaggacaagaatgaggataagaagattgatttttctgagtttctgtccttgctgggagacatagccgcagactaccacaagcagagccatggagcggcgccctgttctgggggaagccagtgatccagccccaccaaggggcctccagagaccccaggaacaataagtgtctcctcccaccagacacttgccttatttcttcttctctttggtgacctacattgtcaaaactaccaattccaggttaactttgttggagaatttcccccacccccatccagtgggtcacccaggagtaatgtccctccagcaacgttccccctatggcctccagcagagctgatctgcctctcacacaggtcctggtgtctgcctctgcaccgttccctaaatgcagccaccttggcaggttccaggtggaagttggtagaaggcccctgccaggtcacagcaatgctctccttgtcaaggcatggaccagggtcattcagacacattcagatactgcactgagaaggagctggcatctctcagtgtgctcctgccctcccactcctgccccagctgttctccagggcttggggaaacagaaaccactcacatagggattcctggatggcttcaggttcagcgcccttggggctatgaatgggaggctcagcagtgccctgaggatgggcttccttgtcctgtggcctctgctccaggggcagtgtcctttccctgtgctgtgtgcttgtgtgcatgtgcctatgtgggtgaccctgtggaagtgagaaggagtcactgtgatgcttagctgtcctaaatgatggtttgctcaatgccaggactgggtttctggtgatgaatgaatattccagattttgaggagctctaagtggtccaggagtccaagtaagcagtctggctggaataaggcagcatcacggaaattctgtaaggactgacacagagagctcatgctgactgtgatgagaaattgcagcacctctatctcgcaggtaatggagtagtttgttattggtagtctactccaggccaggcagtgtgttatgggctgaggatgcagaaacaggcaggacacagtgctgtcctagcagtgcactggcgggtctctccatgcaggccacaacacagggtcagtgttcacctggtgtcacttccaggcaatgttctgtgcagccgctcttagtattcttccttgaggctcacatcatgtgtccctatcactcttactactctggtcagtctccagctaacctctcaatcaggcaaacattcttcttggaggaatcaggcaaacatctcaaaaattctctttccatcctaccagcagcagtgtgtaagatgggctatttgttctttggaatgactgctccactccacactcacacctctattcacagaccagcatctcctctccttatcaggaacattccttcctgaacatattctgcacctcgtcagccttcaggactgatctgccattttcacctctaaatccccatgtctgaccattagttttcttctctttccttctctccctttctcattctcattccacctgttcttggaactcacggagacctacagtccctgggctttcattttctcctcccagccccctgctgccttctctatgcagcctgccctccatcatccaccccagaattgctctctttcctctcttagctctgttgcccactttccttgggccataccttccctgcagatctccagcccagaaccatcttcccctgttgtcctcctctctcctccaccgggactgctggtcactgcttagaaccgtcatgccagggtcccaaaagtgtgggtgcctgacttcctctctgtgcagcactctctgaatccctcctattcaccttgctgctgttattccccgaatgcgcaacataccccccatcaatatatctcagtatttcatgtctcaataccaatcttttaaactactgcctctaccagaaatgtcttttaatacttcttctgtctcattaacattacatttcaaggctgagctttaatgtcagtgtctcttagacattcagagggtgaaccaccatcccttcaccccaaagaaatgatctctgcttcatttgtgcctccctcaccatgaccccactcttaccatagtggctacattacttcagattcccctaatgtctttccagccagacttggaatcatggagggaaaacattgttacctcggatctcctggttacccagcacatagtagtactggattccagctcataataagtactctatatcatttttcaatataaaatgtatttgtgcaaattctagtcaatactactttatgtaaacagcagtgtaaaatccaaaaacttccagtcctggaggcaggttgtgcagcttaggggaggaccccagaatctggaccccagagtctggaagcaggccagaaaggataaggcaaatgactgaacagttccctcaggactcacgtactgatctcccaaaagaagagagggtctccctggggtggggttgctggaccttcaatccatcgctacagtccagaaggcaattggccactcctaatgtgggcctgccctccctttatttttccagttcttatttcacctgataatattccgtccaattggcaatggcacataaaaattaggatggagtgtgtggacaaatacttcttcatcttcttgtctaggttttagaaatcaccttctcaagggagccttgtctaatgttcctgagactatttcacactctccatgcttatgtcaatgcaggactcatcacatctattcggatattctgtttacacacccatgtcatcccagagaggtgatcacagggcagggacacatgtgtggcatacagttcctagttaagatcccaaatcctgagatattgctgatttgctatggcaggtcgtcaagagaactgtgtcattccaaactcaccaaggtggcttatagaacagaagcagatggatatgaagaggagaggggaccagaccatctccgcaaccacagcccagagctccagtcaccagatagaaaattgatttgatttcatccaatattccttcgaaagagtgtcaaggaatagggtggggcaatgtgtcattctgcattggaaggaggacattttagagcaaggcctaagggcacaggtattagtgtcatattgatcagaattcaacctttgttctaacacatactagagcaagaatttacttgatttggaataattaatagctactggacattatattggtactaaagagaaagaatacttgacagctctatgcccacactcacattacagctgatgtgaaagagattctggaaatccaaatgttccccagaaattctgatatcaaaacattccaataacttttttttttcaggcgcagtctcactctgtcgcccgggctggagtgctgtgagctgtccgtggtgctgaattcactgtgacgtcactcctgtctctctttgctttcttctgactgacatttattcagccttctctacaggaatctcttatgttcccccacatgcaggtggtttttcagtaggctcctgaagagtgatctcaactttccaggaagaaaagagggcaaagggaacaatgtgaaaagaagcagaaaatcataaaagaccatgtgtttgataaacaaccagattgtttctggttccctgccactataaaaacaccatgagagcatactcatacatgttcccttataaatctgcgaggtagtttctttggtattcttgcccaggaaatgggttgattcatcacagattttatatatatacttttttttaactaagtgtgagataatatcttattgtttttgtaacttgcattttacaagagttctgaccagcaccagataagcttcagtgctctcctttctttggccttaatattattggattaaagaattactgcctctcactaggagcatcatttatttaccattattttcaatttcatattaaaactcaatttctagtagagtc

SEQ ID NO: 21 - DNA, 1572 bp, Homo sapiens
aatttctcactgcccctgtgataaactgtggtcactggctgtggcagcaactattataagatgctctgaaaactcttcagacactgaggggcaccagaggagcagactacaagaatggcacacgctatggaaaactcctggacaatcagtaaagagtaccatattgatgaagaagtgggctttgctctgccaaatccacaggaaaatctacctgatttttataatgactggatgttcattgctaaacatctgcctgatctcatagagtctggccagcttcgagaaagagttgagaagttaaacatgctcagcattgatcatctcacagaccacaagtcacagcgccttgcacgtctagttctgggatgcatcaccatggcatatgtgtggggcaaaggtcatggagatgtccgtaaggtcttgccaagaaatattgctgttccttactgccaactctccaagaaactggaactgcctcctattttggtttatgcagactgtgtcttggcaaactggaagaaaaaggatcctaataagcccctgacttatgagaacatggacgttttgttctcatttcgtgatggagactgcagtaaaggattcttcctggtctctctattggtggaaatagcagctgcttctgcaatcaaagtaattcctactgtattcaaggcaatgcaaatgcaagaacgggacactttgctaaaggcgctgttggaaatagcttcttgcttggagaaagcccttcaagtgtttcaccaaatccacgatcatgtgaacccaaaagcatttttcagtgttcttcgcatatatttgtctggctggaaaggcaacccccagctatcagacggtctggtgtatgaagggttctgggaagacccaaaggagtttgcagggggcagtgcaggccaaagcagcgtctttcagtgctttgacgtcctgctgggcatccagcagactgctggtggaggacatgctgctcagttcctccaggacatgagaagatatatgccaccagctcacaggaacttcctgtgctcattagagtcaaatccctcagtccgtgagtttgtcctttcaaaaggtgatgctggcctgcgggaagcttatgacgcctgtgtgaaagctctggtctccctgaggagctaccatctgcaaatcgtgactaagtacatcctgattcctgcaagccagcagccaaaggagaataagacctctgaagacccttcaaaactggaagccaaaggaactggaggcactgatttaatgaatttcctgaagactgtaagaagtacaactgagaaatcccttttgaaggaaggttaatgtaacccaacaagagcacattttatcatagcagagacatctgtatgcattcctgtcattacccattgtaacagagccacaaactaatactatgcaatgttttaccaataatgcaatacaaaagacctcaaaatacctgtgcatttcttgtaggaaaacaacaaaaggtaattatgtgtaattatactagaagttttgtaatctgtatcttatcattggaataaaatgacattcaataaataaaaa

SEQ ID NO: 22 - DNA, 1805 bp, Homo sapiens
ctgggaagacgctggtcagttcacctgccccactggttgttttttaaacaaattctgatacaggcgacatcctcactgaccgagcaaagattgacattcgtatcatcactgtgcaccattggcttctaggcactccagtggggtaggagaaggaggtctgaaaccctcgcagagggatcttgccctcattctttgggtctgaaacactggcagtcgttggaaacaggactcagggataaaccagcgcaatggattgggggacgctgcacactttcatcgggggtgtcaacaaacactccaccagcatcgggaaggtgtggatcacagtcatctttattttccgagtcatgatcctcgtggtggctgcccaggaagtgtggggtgacgagcaagaggacttcgtctgcaacacactgcaaccgggatgcaaaaatgtgtgctatgaccactttttcccggtgtcccacatccggctgtgggccctccagctgatcttcgtctccaccccagcgctgctggtggccatgcatgtggcctactacaggcacgaaaccactcgcaagttcaggcgaggagagaagaggaatgatttcaaagacatagaggacattaaaaagcagaaggttcggatagaggggtcgctgtggtggacgtacaccagcagcatctttttccgaatcatctttgaagcagcctttatgtatgtgttttacttcctttacaatgggtaccacctgccctgggtgttgaaatgtgggattgacccctgccccaaccttgttgactgctttatttctaggccaacagagaagaccgtgtttaccatttttatgatttctgcgtctgtgatttgcatgctgcttaacgtggcagagttgtgctacctgctgctgaaagtgtgttttaggagatcaaagagagcacagacgcaaaaaaatcaccccaatcatgccctaaaggagagtaagcagaatgaaatgaatgagctgatttcagatagtggtcaaaatgcaatcacaggtttcccaagctaaacatttcaaggtaaaatgtagctgcgtcataaggagacttctgtcttctccagaaggcaataccaacctgaaagttccttctgtagcctgaagagtttgtaaatgactttcataataaatagacacttgagttaactttttgtaggatacttgctccattcatacacaacgtaatcaaatatgtggtccatctctgaaaacaagagactgcttgacaaaggagcattgcagtcactttgacaggttccttttaagtggactctctgacaaagtgggtactttctgaaaatttatataactgttgttgataaggaacatttatccaggaattgatacttttattaggaaaagatatttttataggcttggatgtttttagttctgactttgaatttatataaagtatttttataatgactggtcttccttacctggaaaaacatgcgatgttagttttagaattacaccacaagtatctaaatttggaacttacaaagggtctatcttgtaaatattgttttgcattgtctgttggcaaatttgtgaactgtcatgatacgcttaaggtggaaagtgttcattgcacaatatatttttactgctttctgaatgtagacggaacagtgtggaagcagaaggcttttttaactcatccgtttgccaatcattgcaaacaactgaaatgtggatgtgattgcctcaataaagctcgtccccattgcttaagccttcaaaaaaaaaaaaaaaaaaaaaaaaaaaaaaaaaaaaaaa

SEQ ID NO: 23 - DNA, 1302 bp, Homo sapiens
gagacagcccgccggccgcccggatctccacctgccaccccagagctgggacagcagccgggctgcggcactgggagggagaccccacagtggcctcttctgccacccacgcccccacccctggcatggccgaccagctgactgaggagcaggtcacagaattcaaggaggccttctccctgtttgacaaggatggggacggctgcatcaccacccgcgagctgggcacggtcatgcggtccctgggccagaaccccacggaggccgagctgcgggacatgatgagtgagatcgaccgggacggcaacggcaccgtggacttccccgagttcctgggcatgatggccaggaagatgaaggacacggacaacgaggaggagatccgcgaggccttccgcgtgttcgacaaggacggcaacggcttcgtcagcgccgccgagctgcgacacgtcatgacccggctgggggagaagctgagtgacgaggaggtggacgagatgatccgggccgcggacacggacggagacggacaggtgaactacgaggagtttgtccgtgtgctggtgtccaagtgaggccggcgcccaccatgctcctgggcgcccacgcggcccacagggcaagaacccggggcctcccgcctcctcccccatccccctgcctcccctgggcactgtggcttcctcctgcgcctggttgattcagcccacctctctgcatcccgcttcccgcgtctcttctctgcactcctgccgaccttcccacctgctcatctgaatgacacggaacgctcccactgcaggcaaaccgtgacgccctccccactcgggagaagcagagctgaccttaggaccgagcaccagggcaggttgcgctgactctgcggccctccaggacggacaccgggtgaccccttagggcacccaggcaagatccctaagaggcacccaatgcccaggccaggggggctgcagccctcagcccccgccaggattcccgcaggctcctggactggaagctccctccgcggtcggattctggagggtgggaggcatcttggcctgcagtaagcggtgctgacggggactctggccacagaggtcaggcctcctgaaaacagcactgccttccgcgctgccccagcttgccccattccttgtccgccaacccaccgtgattcatcttctgaagctgggagtgaaactgggtcagctgtaacctgttcctattcatctggaaggagggaggcttggatgagcaggggatgagagctgcagggaaataaatgagatattcgtcctt

SEQ ID NO: 24 - DNA, 1694 bp, Homo sapiens
ctctctgcccacctctgcttcctctaggaacacaggagttccagatcacatcgagttcaccatgaattcactcagtgaagccaacaccaagttcatgttcgacctgttccaacagttcagaaaatcaaaagagaacaacatcttctattcccctatcagcatcacatcagcattagggatggtcctcttaggagccaaagacaacactgcacaacagattaagaaggttcttcactttgatcaagtcacagagaacaccacaggaaaagctgcaacatatcatgttgataggtcaggaaatgttcatcaccagtttcaaaagcttctgactgaattcaacaaatccactgatgcatatgagctgaagatcgccaacaagctcttcggagaaaaaacgtatctatttttacaggaatatttagatgccatcaagaaattttaccagaccagtgtggaatctgttgattttgcaaatgctccagaagaaagtcgaaagaagattaactcctgggtggaaagtcaaacgaatgaaaaaattaaaaacctaattcctgaaggtaatattggcagcaataccacattggttcttgtgaacgcaatctatttcaaagggcagtgggagaagaaatttaataaagaagatactaaagaggaaaaattttggccaaacaagaatacatacaagtccatacagatgatgaggcaatacacatcttttcattttgcctcgctggaggatgtacaggccaaggtcctggaaataccatacaaaggcaaagatctaagcatgattgtgttgctgccaaatgaaatcgatggtctccagaagcttgaagagaaactcactgctgagaaattgatggaatggacaagtttgcagaatatgagagagacacgtgtcgatttacacttacctcggttcaaagtggaagagagctatgacctcaaggacacgttgagaaccatgggaatggtggatatcttcaatggggatgcagacctctcaggcatgaccgggagccgcggtctcgtgctatctggagtcctacacaaggcctttgtggaggttacagaggagggagcagaagctgcagctgccaccgctgtagtaggattcggatcatcacctacttcaactaatgaagagttccattgtaatcaccctttcctattcttcataaggcaaaataagaccaacagcatcctcttctatggcagattctcatccccgtagatgcaattagtctgtcactccatttggaaaatgttcacctgcagatgttctggtaaactgattgctggcaacaacagattctcttggctcatatttcttttctttctcatcttgatgatgatcgtcatcatcaagaatttaatgattaaaatagcatgcctttctctctttctcttaataagcccacatataaatgtactttttcttccagaaaaattctccttgaggaaaaatgtccaaaataagatgaatcacttaataccgtatcttctaaatttgaaatataattctgtttgtgacctgttttaaatgaaccaaaccaaatcatactttttctttgaatttagcaacctagaaacacacatttctttgaatttaggtgatacctaaatccttcttatgtttctaaattttgtgattctataaaacacatcatcaataaaatagtgacataaaatca

SEQ ID NO: 25 - DNA, 1539 bp, Homo sapiens
ccagtctccgcgcctccacccagctcaggaacccgcgaaccctctcttgaccactatgagcctcccgtccagccgcgcggcccgtgtcccgggtccttcgggctccttgtgcgcgctgctcgcgctgctgctcctgctgacgccgccggggcccctcgccagcgctggtcctgtctctgctgtgctgacagagctgcgttgcacttgtttacgcgttacgctgagagtaaaccccaaaacgattggtaaactgcaggtgttccccgcaggcccgcagtgctccaaggtggaagtggtagcctccctgaagaacgggaagcaagtttgtctggacccggaagccccttttctaaagaaagtcatccagaaaattttggacagtggaaacaagaaaaactgagtaacaaaaaagaccatgcatcataaaattgcccagtcttcagcggagcagttttctggagatccctggacccagtaagaataagaaggaagggttggtttttttccattttctacatggattccctactttgaagagtgtgggggaaagcctacgcttctccctgaagtttacagctcagctaatgaagtactaatatagtatttccactatttactgttattttacctgataagttattgaaccctttggcaattgaccatattgtgagcaaagaatcactggttattagtctttcaatgaatattgaattgaagataactattgtatttctatcatacattccttaaagtcttaccgaaaaggctgtggatttcgtatggaaataatgttttattagtgtgctgttgagggaggtatcctgttgttcttactcactcttctcataaaataggaaatattttagttctgttttcttggggaatatgttactctttaccctaggatgctatttaagttgtactgtattagaacactgggtgtgtcataccgttatctgtgcagaatatatttccttattcagaatttctaaaaatttaagttctgtaagggctaatatattctcttcctatggttttagatgtttgatgtcttcttagtatggcataatgtcatgatttactcattaaactttgattttgtatgctattttttcactataggatgactataattctggtcactaaatatacactttagatagatgaagaagcccaaaaacagataaattcctgattgctaatttacatagaaatgtattctcttggttttttaaataaaagcaaaattaacaatgatctgtgctctgcaaagttttgaaaatatatttgaacaatttgaatataaattcatcatttagtcctcaaaatatatacagcattgctaagattttcagatatctattgtggatcttttaaaggttttgaccattttgttatgaggaattatacatgtatcacattcactatattaaaattgcacttttattttttcctgtgtgtcatgttggtttttggtacttgtattgtcatttggagaaacaataaaagatttctaaaccaaaaaaaaaaaaaaaaaaa

SEQ ID NO: 26 - DNA, 2844 bp, Homo sapiens
atgaggcccggcctctcatttctcctagcccttctgttcttccttggccaagctgcaggggatttgggggatgtgggacctccaattcccagccccggcttcagctctttcccaggtgttgactccagctccagcttcagctccagctccaggtcgggctccagctccagccgcagcttaggcagcggaggttctgtgtcccagttgttttccaatttcaccggctccgtggatgaccgtgggacctgccagtgctctgtttccctgccagacaccacctttcccgtggacagagtggaacgcttggaattcacagctcatgttctttctcagaagtttgagaaagaactttccaaagtgagggaatatgtccaattaattagtgtgtatgaaaagaaactgttaaacctaactgtccgaattgacatcatggagaaggataccatttcttacactgaactggacttcgagctgatcaaggtagaagtgaaggagatggaaaaactggtcatacagctgaaggagagttttggtggaagctcagaaattgttgaccagctggaggtggagataagaaatatgactctcttggtagagaagcttgagacactagacaaaaacaatgtccttgccattcgccgagaaatcgtggctctgaagaccaagctgaaagagtgtgaggcctctaaagatcaaaacacccctgtcgtccaccctcctcccactccagggagctgtggtcatggtggtgtggtgaacatcagcaaaccgtctgtggttcagctcaactggagagggttttcttatctatatggtgcttggggtagggattactctccccagcatccaaacaaaggactgtattgggtggcgccattgaatacagatgggagactgttggagtattatagactgtacaacacactggatgatttgctattgtatataaatgctcgagagttgcggatcacctatggccaaggtagtggtacagcagtttacaacaacaacatgtacgtcaacatgtacaacaccgggaatattgccagagttaacctgaccaccaacacgattgctgtgactcaaactctccctaatgctgcctataataaccgcttttcatatgctaatgttgcttggcaagatattgactttgctgtggatgagaatggattgtgggttatttattcaactgaagccagcactggtaacatggtgattagtaaactcaatgacaccacacttcaggtgctaaacacttggtataccaagcagtataaaccatctgcttctaacgccttcatggtatgtggggttctgtatgccacccgtactatgaacaccagaacagaagagattttttactattatgacacaaacacagggaaagagggcaaactagacattgtaatgcataagatgcaggaaaaagtgcagagcattaactataacccttttgaccagaaactttatgtctataacgatggttaccttctgaattatgatctttctgtcttgcagaagccccagtaagctgtttaggagttagggtgaaagagaaaatgtttgttgaaaaaatagtcttctccacttacttagatatctgcaggggtgtctaaaagtgtgttcattttgcagcaatgtttaggtgcatagttctaccacactagagatctaggacatttgtcttgatttggtgagttctcttgggaatcatctgcctcttcaggcgcattttgcaataaagtctgtctagggtgggattgtcagaggtctaggggcactgtgggcctagtgaagcctactgtgaggaggcttcactagaagccttaaattaggaattaaggaacttaaaactcagtatggcgtctagggattctttgtacaggaaatattgcccaatgactagtcctcatccatgtagcaccactaattcttccatgcctggaagaaacctggggacttagttaggtagattaatatctggagctcctcgagggaccaaatctccaacttttttttcccctcactagcacctggaatgatgctttgtatgtggcagataagtaaatttggcatgcttatatattctacatctgtaaagtgctgagttttatggagagaggcctttttatgcattaaattgtacatggcaaataaatcccagaaggatctgtagatgaggcacctgctttttcttttctctcattgtccaccttactaaaagtcagtagaatcttctacctcataacttccttccaaaggcagctcagaagattagaaccagacttactaaccaattccaccccccaccaacccccttctactgcctactttaaaaaaattaatagttttctatggaactgatctaagattagaaaaattaattttctttaatttcattatgaacttttatttacatgactctaagactataagaaaatctgatggcagtgacaaagtgctagcatttattgttatctaataaagaccttggagcatatgtgcaacttatgagtgtatcagttgttgcatgtaatttttgcctttgtttaagcctggaacttgtaagaaaatgaaaatttaatttttttttctaggacgagctatagaaaagctattgagagtatctagttaatcagtgcagtagttggaaaccttgctggtgtatgtgatgtgcttctgtgcttttgaatgactttatcatctagtctttgtctatttttcctttgatgttcaagtcctagtctataggattggcagtttaaatgctttactcccccttttaaaataaatgattaaaatgtgctttgaaaaaaaaaaaaaaaaaaaaaaaaaaaaaaa

SEQ ID NO: 27 - DNA, 1577 bp, Homo sapiens
ggctgaggcaacctgaaggaggagctctcattaccttctgcccatcacttaataaatagccagccaattcatcaacattctggtacactgttggagagatgagacagtcacaccagctgcccctagtggggctcttactgttttcttttattccaagccaactatgcgagatttgtgaggtaagtgaagaaaactacatccgcctaaaacctctgttgaatacaatgatccagtcaaactataacaggggaaccagcgctgtcaatgttgtgttgtccctcaaacttgttggaatccagatccaaaccctgatgcaaaagatgatccaacaaatcaaatacaatgtgaaaagcagattgtcagatgtaagctcgggagagcttgccttgattatactggctttgggagtatgtcgtaacgctgaggaaaacttaatatatgattaccacctgatcgacaagctagaaaataaattccaagcagaaattgaaaatatggaagcacacaatggcactcccctgactaactactaccagctcagcctggacgttttggccttgtgtctgttcaatgggaactactcaaccgccgaagttgtcaaccacttcactcctgaaaataaaaactattattttggtagccagttctcagtagatactggtgcaatggctgtcctggctctgacctgtgtgaagaagagtctaataaatgggcagatcaaagcagatgaaggcagtttaaagaacatcagtatttatacaaagtcactggtagaaaagattctgtctgagaaaaaagaaaatggtctcattggaaacacatttagcacaggagaagccatgcaggccctctttgtatcatcagactattataatgaaaatgactggaattgccaacaaactctgaatacagtgctcacggaaatttctcaaggagcattcagcaatccaaacgctgcagcccaggtcttacctgccctgatgggaaagaccttcttggatattaacaaagactcttcttgcgtctctgcttcaggtaacttcaacatctccgctgatgagcctataactgtgacacctcctgactcacaatcatatatctccgtcaattactctgtgagaatcaatgaaacatatttcaccaatgtcactgtgctaaatggttctgtcttcctcagtgtgatggagaaagcccagaaaatgaatgatactatatttggtttcacaatggaggagcgctcatgggggccctatatcacctgtattcagggcctatgtgccaacaataatgacagaacctactgggaacttctgagtggaggcgaaccactgagccaaggagctggtagttacgttgtccgcaatggagaaaacttggaggttcgctggagcaaatactaataagcccaaactttcctcagctgcataaaatccatttgcagtggagttccatgtttattgtccttatgccttcttcttcatttatcccagtacgagcaggagagttaataacctccccttctctctctacatgttcaataaaagttgttgaaagattaacaactataaaaaaaaaaa

SEQ ID NO: 28 - DNA, 2014 bp, Homo sapiens
aggcggcttttggtcacaggctcccgagttctcctagctggggctgcggagctggggggagggaagagaggaaaggggagggggtgcctggagaggcggaggctcgcgcgcctgcgcatccagctccagggaccctaggttttctatgggattcccaatctgcagcagagatttacccgagcgtgttgcggcagcggctgggcttgcaaggcgcgatccaagagggatttaagcagcccagagctccagagaaaaagagagcgagagagaaccacacacagagacggcttaagcgtttacccgaattaaatatatatttttaaaaagaactgttgagttttatcattttcgttaagtgaccgtgcgcagcgctgtaactgcaggatggggaagcagaatagcaaactggcccctgaagtgatggaggacctggtgaagagcacagagtttaatgagcatgaactcaagcagtggtacaaaggatttctcaaggactgtccaagtgggaggctaaatctcgaggaatttcagcagctctatgtgaagttctttccttatggagacgcctccaagtttgcccagcatgccttccgaaccttcgacaagaatggggacggcaccattgacttccgagagttcatctgcgctctgtccatcacctccaggggcagctttgagcagaagctgaactgggccttcaatatgtatgacctggatggtgatggcaagatcacccgagtggagatgctggagatcatcgaggctatctacaaaatggtaggcactgtgatcatgatgaaaatgaatgaggatggcctgacgcctgagcagcgagtagacaagattttcagcaagatggataagaacaaagatgaccagattacactggatgaattcaaagaagctgcaaagagcgacccttccattgtattacttctgcagtgcgacatccagaaatgagctgatgtcaatgctatggactgcacaaaagtctcaatgttccattcagtctgcagctattcacacacacacacacacacacacacacacacacacacacacacacaaatattgcttggactacctataaatggacttgcttcttgtgtttgaaacactcgtgtgcatgagaatgtcatttgctaatgaattttaaaagcatatataaaacaaaacaaacaacctgccacaatgtgatatgtgtaatatcatttcataaaaatccctcttcctccaaagcctgggcagaaatgtgctgcaaagagttatatgacttcttgttcatgttttgctaatgctcgtatctccttgattacataatgttagtagcactgagacccccatggtaatgtaacttaattataagctatgtcactaccctcctgtaaaatactattggacagacacagagggacccttggctcctgtgtctggtccacacaccacagaagcttgtattatcagtgaatataaatgtactacatttgcatgccttttgggtttgccttaattcttacctcatttgcatcctatcgatctggaaagagctgttttggatgaatgcagtataaaatgtaaaaaccctgctaaatgacttattgattaagtatatctatctatatatacatatacacaaagatattatttatcgaaagtaaaaaagatggaagtgtattggtttctgtttgaattttcaaaggcttccaatgtggtggcaataaatgtcccaaataaatttataacaattgattttccccctaattcttattttataattttaaaattgcagcagttgctagcaacaacttactaaatctactcttaaatatacaactttggaatttgaagaattaatgacaacaaaagggaaaaaagcaactttccaacttttcatccaggctcccaaaagagggacaacgaacatggcatgtgaaaagtaaaacagatttgttcattccgaaaaaaaaatgttcattctatgacaataaattttatctcagtgtgaaaaaaaaaa

SEQ ID NO: 29 - DNA, 971 bp, Homo sapiens
gattgcttgaggagagaagtatgtgatcagaaagcattctttgtctattaactcctgcccagcaaaagtgaaagaaaattcatgggagcatgcaagaacaaagagcacagcaaagctggacaaacacagcaatccaggcaggggatttccaactcaactctggtatgtaagctgcatgcaaagtcctttttctgtctctggtttctggccccttgtctgcagagatggctcccaatgcttcctgcctctgtgtgcatgtccgttccgaggaatgggatttaatgacctttgatgccaacccatatgacagcgtgaaaaaaatcaaagaacatgtccggtctaagaccaaggttcctgtgcaggaccaggttcttttgctgggctccaagatcttaaagccacggagaagcctctcatcttacggcattgacaaagagaagaccatccaccttaccctgaaagtggtgaagcccagtgatgaggagctgcccttgtttcttgtggagtcaggtgatgaggcaaagaggcacctcctccaggtgcgaaggtccagctcagtggcacaagtgaaagcaatgatcgagactaagacgggtataatccctgagacccagattgtgacttgcaatggaaagagactggaagatgggaagatgatggcagattacggcatcagaaagggcaacttactcttcctggcatcttattgtattggagggtgaccaccctgggcatggggtgttggcaggggtcaaaaagcttatttcttttaatctcttactcaacgaacacatcttctgatgatttcccaaaattaatgagaatgagatgagtagagtaagatttgggtgggatgggtaggatgaagtatattgcccaactctatgtttctttgattctaacacaattaattaagtgacatgatttttactaatgtattactgagactagtaaataaatttttaagccaa

SEQ ID NO: 30 - DNA, 1485 bp, Homo sapiens
tcagccaattagagctccagttgtcactcctacccacactgggcctgggggtgaagggaagtgtttattaggggtacatgtgaagccgtccagaagtgtcagagtctttgtagctttgaaagtcacctaggttatttgggcatgctctcctgagtcctctgctagttaagctctctgaaaagaaggtggcagacccggtttgctgatcgccccagggatcaggaggctgatcccaaagttgtcagatggagagtaaatacaaggagatactcttgctaacaggcctggataacatcactgatgaggaactggataggtttaagttctttctttcagacgagtttaatattgccacaggcaaactacatactgcaaacagaatacaagtagctaccttgatgattcaaaatgctggggcggtgtctgcagtgatgaagaccattcgtatttttcagaagttgaattatatgcttttggcaaaacgtcttcaggaggagaaggagaaagttgataagcaatacaaatcggtaacaaaaccaaagccactaagtcaagctgaaatgagtcctgctgcatctgcagccatcagaaatgatgtcgcaaagcaacgtgctgcaccaaaagtctctcctcatgttaagcctgaacagaaacagatggtggcccagcaggaatctatcagagaagggtttcagaagcgctgtttgccagttatggtactgaaagcaaagaagcccttcacgtttgagacccaagaaggcaagcaggagatgtttcatgctacagtggctacagaaaaggaattcttctttgtaaaagtttttaatacactgctgaaagataaattcattccaaagagaataattataatagcaagatattatcggcacagtggtttcttagaggtaaatagcgcctcacgtgtgttagatgctgaatctgaccaaaaggttaatgtcccgctgaacattatcagaaaagctggtgaaaccccgaagatcaacacgcttcaaactcagccccttggaacaattgtgaatggtttgtttgtagtccagaaggtaacagaaaagaagaaaaacatattatttgacctaagtgacaacactgggaaaatggaagtactgggggttagaaacgaggacacaatgaaatgtaaggaaggagataaggttcgacttacattcttcacactgtcaaaaaatggagaaaaactacagctgacatctggagttcatagcaccataaaggttattaaggccaaaaaaaaaacatagagaagtaaaaaggaccaattcaagccaactggtctaagcagcatttaattgaagaatatgtgatacagcctcttcaatcagattgtaagttacctgaaagctgcagttcacaggctcctctctccaccaaattaggatagaataattgctggataaacaaattcagaatatcaacagatgatcacaataaacatctgtttctcattcc

SEQ ID NO: 31 - DNA, 4650 bp, Homo sapiens
atgagcctttcattttgtggtaacaacatttcttcatataatatcaacgatggtgtactacaaaattcctgctttgtggatgccctcaacctggtccctcatgtctttctgttgtttatcacttttccaatattgtttattgggtgggggagccaaagctcaaaagtacaaattcaccacaacacatggcttcattttccgggacataacctgagatggattcttacattcgctctcctgtttgtgcatgtctgtgaaatagcagaaggcattgtttcagactcgcggcgggaatcaaggcacctccacctctttatgccagccgtgatgggattcgttgccactacaacatcgatagtgtattatcataatatcgaaacatcaaattttcctaaattacttttagccctgttcctgtattgggtaatggcctttattacaaaaacaataaaattggttaagtactgtcagtctggcttggacatatcaaacctgcgtttctgcatcacaggcatgatggtcatcttgaatgggctcttgatggctgtggagatcaatgtcattcgagtcaggagatatgtatttttcatgaatcctcagaaagtaaagcctcctgaagacctccaggatctgggagtgagatttcttcaaccatttgtgaatttgctgtcaaaagcaacatactggtggatgaacacacttattatatctgctcacaaaaagcctattgatctgaaggcaattggaaaattgccaatagcaatgagagcagtaacaaattatgtttgcctgaaagatgcatatgaagaacaaaagaaaaaagttgcagatcatccaaatcggactccatctatatggcttgcaatgtacagagcttttgggcgaccaattctacttagtagcacattccgctatctggctgatttactgggttttgctggacctctttgtatttctggaatagttcagcgtgtgaatgaaacccagaatgggacaaataacacaactggaatttcagaaaccctctcatcaaaggaatttcttgaaaacgcttacgttctagcagttcttctcttcttggctcttattctgcaaaggacatttttgcaggcttcctactatgtaaccatagagactggcattaacctccgtggagctctgctggccatgatttataataaaatccttaggctctctacgtctaacttatccatgggggagatgactctggggcagatcaacaacttagtcgccattgaaactaatcaactcatgtggtttttgttcctgtgtcccaatctatgggctatgcctgttcagatcataatgggcgtgattctgctctataatttacttggatcaagtgcattggtcggtgcagctgtcattgtgctccttgcgccaattcagtactttattgctacaaagttggcagaggctcagaaaagtacacttgattattccactgagagactcaagaaaacaaatgaaatattgaaaggcatcaaacttctaaaattgtatgcctgggaacacattttctgcaaaagtgtggaggaaacaagaatgaaagaactatctagtctcaaaacctttgcactatatacatcactctccatcttcatgaatgcagcaattcccatagcagctgttcttgctacatttgtgacccatgcgtatgccagtggaaacaatctgaaacctgcagaggcctttgcttcactgtctctcttccatatcctggtcacaccactgtccctgctcttcacggtggtcagatttgcagtcaaagccatcataagtgttcaaaagctgaatgagtttctcttgagtgatgagattggtgacgacagttggcgaactggtgaaagttcgcttccttttgagtcctgtaagaagcacactggagttcagccaaaaactataaacaggaaacagcctggaagatatcacctggacagctatgagcaatcaacacggcgtctacgtcccgcagaaacagaggacattgcaataaaggtcacaaatggatacttttcatggggcagtggtttagctacattatccaatatagatattcgaattccaacaggtcagttaaccatgattgtgggccaagtaggatgtgggaagtcctctcttctccttgccatcctcggtgagatgcagacattggaaggaaaagttcactggagcaatgtaaatgaatctgagccttcttttgaagcaaccagaagtaggaacaggtactctgtggcatatgcagctcaaaagccttggctattaaatgctacagtagaagaaaatattacttttggaagtccttttaacaaacagaggtacaaagctgtcacagatgcctgttctcttcagccagatattgacttattaccatttggagatcaaactgaaattggagagaggggcatcaacctgagtgggggacagaggcagagaatctgtgtggcacgagcgctgtatcaaaacaccaacattgtctttttggatgatccattctcagccctggacattcacttgagtgatcatttaatgcaggaggggattttgaaattcctgcaagatgacaaaaggacactcgttcttgtgactcacaaattacagtatctgacgcatgctgactggatcatagccatgaaagatggaagtgtcctaagagaaggaactttgaaggacattcaaaccaaagatgttgagctttatgaacactggaaaacacttatgaatcggcaagatcaagaattagaaaaggatatggaagctgaccaaactactttagagaggaaaactctccgacgggccatgtattcaagagaagccaaagcccagatggaggacgaagacgaagaggaagaagaggaggaagatgaggatgataacatgtccactgtaatgaggctcaggactaaaatgccatggaaaacctgctggcgctacctgacatctggaggattcttcctgctcatcctgatgattttctctaagcttttgaagcattcggtcattgtagctatagactattggctggccacatggacatcggagtacagtataaacaatactggaaaagctgatcagacctactatgtggctggctttagcatactctgtggagcaggcattttcctttgccttgttacatccctcactgtagaatggatgggtctcacagctgccaaaaatcttcaccacaaccttctcaataagataatccttggaccaataaggttttttgataccacacccctgggactgattctcaatcgcttttcagctgatactaatatcattgatcagcacatccctccaaccttggaatctctaactcgctcaacactgctctgcctgtctgccattgggatgatttcttatgctactcctgtgttcctggttgctctcctgccccttggtgttgccttttattttatccagaaatactttcgggttgcctctaaggacctccaggaactcgacgatagtacccagctccctctgctctgtcacttctcagaaacagcagaaggactcaccaccattcgggcctttaggcatgaaaccagatttaaacaacgtatgctggaactgacggatacaaacaacattgcctacttatttctctcagctgccaacagatggctggaggtcaggacggattatctgggagcttgcattgtcctcactgcatctatagcatccattagtgggtcttccaattctggattggtaggcttgggtcttctgtatgcacttacgataaccaattatttgaattgggttgtgaggaacttggctgacctggaggtccagatgggtgcagtgaagaaggtgaacagtttcctgactatggagtcagagaactatgaaggcacaatggatccttctcaagttccagaacattggccacaagaaggggagatcaagatacatgatctgtgtgtcagatatgaaaataatctgaaacctgttcttaagcacgtcaaggcttacatcaaacctggacaaaaggtgggcatatgtggtcgcactggcagtgggaaatcatcgttatctctggctttcttcagaatggttgatatatttgatggaaaaattgtcattgatgggatagacatttccaaattaccactgcacacactacgttctagactttcaatcattctgcaggatccaatactattcagtggttccattagatttaatttagatccagagtgcaaatgcacagatgacagactctgggaagccttagaaattgctcagctgaagaatatggtcaaatctctacctggaggtctagatgcggttgtcactgaaggtggggagaattttagcgtgggacagagacagctattttgccttgccagggcctttgtccgcaaaagcagcattcttattatggatgaggcaacagcttccattgacatggccacagagaatattttgcaaaaagtagtaatgacagcctttgcagaccggaccgtggtgacaatggctcatcgagtacacactattctgacggcagacctggttattgtgatgaagcgaggaaatattttagaatatgacactccagaaagcctcttggctcaggaaaatggagtatttgcttcttttgttcgcgcagacatgtga

SEQ ID NO: 32 - DNA, 3143 bp, Homo sapiens
ctataaattaaggatcccagctacttaattgacttatgcttcctagttcgttgcccagccaccaccgtctctccaaaaacccgaggtctcgctaaaatcatcatggattcacttggcgccgtcagcactcgacttgggtttgatcttttcaaagagctgaagaaaacaaatgatggcaacatcttcttttcccctgtgggcatcttgactgcaattggcatggtcctcctggggacccgaggagccaccgcttcccagttggaggaggtgtttcactctgaaaaagagacgaagagctcaagaataaaggctgaagaaaaagaggtgattgagaacacagaagcagtacatcaacaattccaaaagtttttgactgaaataagcaaactcactaatgattatgaactgaacataaccaacaggctgtttggagaaaaaacatacctcttccttcaaaaatacttagattatgttgaaaaatattatcatgcatctctggaacctgttgattttgtaaatgcagccgatgaaagtcgaaagaagattaattcctgggttgaaagcaaaacaaatgaaaaaatcaaggacttgttcccagatggctctattagtagctctaccaagctggtgctggtgaacatggtttattttaaagggcaatgggacagggagtttaagaaagaaaatactaaggaagagaaattttggatgaataagagcacaagtaaatctgtacagatgatgacacagagccattcctttagcttcactttcctggaggacttgcaggccaaaattctagggattccatataaaaacaacgacctaagcatgtttgtgcttctgcccaacgacatcgatggcctggagaagataatagataaaataagtcctgagaaattggtagagtggactagtccagggcatatggaagaaagaaaggtgaatctgcacttgccccggtttgaggtggaggacggttacgatctagaggcggtcctggctgccatggggatgggcgatgccttcagtgagcacaaagccgactactcgggaatgtcgtcaggctccgggttgtacgcccagaagttcctgcacagttcctttgtggcagtaactgaggaaggcaccgaggctgcagctgccaccggcataggctttactgtcacatccgccccaggtcatgaaaatgttcactgcaatcatcccttcctgttcttcatcaggcacaatgaatccaacagcatcctcttcttcggcagattttcttctccttaagatgatcgttgccatggcattgctgcttttagcaaaaaacaactaccagtgttactcatatgattatgaaaatcgtccattcttttaaatgttgtctcacttgcatttccagtcttggccatcaaatcaatgatttaatgactccaataatgtgtgtgtttataaccatcctcgaaagtgaaatgtccttttttttgtgccatgcgtaaggtgagtcaaaccaaacctcattgataatctcccctttggtttcctttgaaagtaaattggtatcttgtagttttgtgcacacgaaaggagagaaagtttctccagtaaagagtacgaactagtaattttggggggtctctctaattctggtattttgacatgttataatacgcaagtaaaataaaacaatagtttactcagctcatgttactattccccaacagatattgtggcaaatcacacataggaaagaggatttgggaatacagtagcaaaacataaattaaaactcaaatgcccaggacaaaataaaacaatataccagatggagaggatgcccgtattttcatcttccattctaacattatccattgttagatgcataagcattttgatattgtgtaataaatgtggtatttgagaagataaatgatgtagttgatcagtaatcctcctctatcacctttttagactttgtaaggtaaatatttggactaacttttagaaaagtttccctttttttctccatttacatttttctggtttttttttttttttttgagtgaggtacgagtattaccaaatgatattttctgaagatgctttttggaaagctctgaatctatacctaatgctcttaattattggcttgtttcatttttttcctccagtttttaacaagatcacataactggcttatttttaacagctttgtcaaactacaatttacatgccgtaaaatgtacacactgtaattttataattcattgacttttagtaaattttctagcgttatgcatcgccacaatccagttttagaatatttccatgaccctaagaagtttcctcatgtctattaatattcccaatcctaggcaccactgagttgttttctgtctttataagtttttctttctacatcttatataaatggaatcataatacatgtagtattttgtgtctggcgtcttgcacttagcatggtgttcttgaggttcatctgttgtagtatgtattgatacttaggatttttttattgccgaatactattccattgcatggaaaagacctattttatttctaggttcaccagttgagggacatttggattgttcccacttcttgggctgttaggaataatgttgctctgaacatgtaaataaagatctttgtgttcacatatgtttttcattttctgttggggagattccctaggctagaaattgctgggccatatgaaaaatcaatagttagctttgtaagaaacagtcaaactgttttcccaacgtgacattttatattcccaccaggaatgtttaaaactagtgtcttcaaatcctcaccaacatccaggattgtgtctttatgattatagccatttttgtaggtacaaagtggcatctcatggtggttttaatttgcatttccataatatctaattaggttgagctttttttatgtgcttattggccatttgtttgactttgtttggtgaaatgtatacaaatcatttgctcatttttaatttgggttgtctgtcttgtcttctcattttattgagttaaatgagttcttaataatctctggcttacaagtccttaatttatcaaatatatgatacgtggacatttcctcataaaaaaaaaaaaaaaaaa

SEQ ID NO: 33 - DNA, 1652 bp, Homo sapiens
aatttctcactgcccctgtgataaactgtggtcactggctgtggcagcaactattataagatgctctgaaaactcttcagacactgaggggcaccagaggagcagactacaagaatggcacacgctatggaaaactcctggacaatcagtaaagagtaccatattgatgaagaagtgggctttgctctgccaaatccacaggaaaatctacctgatttttataatgactggatgttcattgctaaacatctgcctgatctcatagagtctggccagcttcgagaaagagttgagaagttaaacatgctcagcattgatcatctcacagaccacaagtcacagcgccttgcacgtctagttctgggatgcatcaccatggcatatgtgtggggcaaaggtcatggagatgtccgtaaggtcttgccaagaaatattgctgttccttactgccaactctccaagaaactggaactgcctcctattttggtttatgcagactgtgtcttggcaaactggaagaaaaaggatcctaataagcccctgacttatgagaacatggacgttttgttctcatttcgtgatggagactgcagtaaaggattcttcctggtctctctattggtggaaatagcagctgcttctgcaatcaaagtaattcctactgtattcaaggcaatgcaaatgcaagaacgggacactttgctaaaggcgctgttggaaatagcttcttgcttggagaaagcccttcaagtgtttcaccaaatccacgatcatgtgaacccaaaagcatttttcagtgttcttcgcatatatttgtctggctggaaaggcaacccccagctatcagacggtctggtgtatgaagggttctgggaagacccaaaggagtttgcagggggcagtgcaggccaaagcagcgtctttcagtgctttgacgtcctgctgggcatccagcagactgctggtggaggacatgctgctcagttcctccaggacatgagaagatatatgccaccagctcacaggaacttcctgtgctcattagagtcaaatccctcagtccgtgagtttgtcctttcaaaaggtgatgctggcctgcgggaagcttatgacgcctgtgtgaaagctctggtctccctgaggagctaccatctgcaaatcgtgactaagtacatcctgattcctgcaagccagcagccaaaggagaataagacctctgaagacccttcaaaactggaagccaaaggaactggaggcactgatttaatgaatttcctgaagactgtaagaagtacaactgagaaatcccttttgaaggaaggttaatgtaacccaacaagagcacattttatcatagcagagacatctgtatgcattcctgtcattacccattgtaacagagccacaaactaatactatgcaatgttttaccaataatgcaatacaaaagacctcaaaatacctgtgcatttcttgtaggaaaacaacaaaaggtaattatgtgtaattatactagaagttttgtaatctgtatcttatcattggaataaaatgacattcaataaataaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 34 - DNA, 2320 bp, Homo sapiens
tcgacagctctctcgcccagcccagttctggaagggataaaaagggggcatcaccgttcctgggtaacagagccaccttctgcgtcctgctgagctctgttctctccagcacctcccaacccactagtgcctggttctcttgctccaccaggaacaagccaccatgtctcgccagtcaagtgtgtccttccggagcgggggcagtcgtagcttcagcaccgcctctgccatcaccccgtctgtctcccgcaccagcttcacctccgtgtcccggtccgggggtggcggtggtggtggcttcggcagggtcagccttgcgggtgcttgtggagtgggtggctatggcagccggagcctctacaacctggggggctccaagaggatatccatcagcactagtggtggcagcttcaggaaccggtttggtgctggtgctggaggcggctatggctttggaggtggtgccggtagtggatttggtttcggcggtggagctggtggtggctttgggctcggtggcggagctggctttggaggtggcttcggtggccctggctttcctgtctgccctcctggaggtatccaagaggtcactgtcaaccagagtctcctgactcccctcaacctgcaaatcgaccccagcatccagagggtgaggaccgaggagcgcgagcagatcaagaccctcaacaataagtttgcctccttcatcgacaaggtgcggttcctggagcagcagaacaaggttctggacaccaagtggaccctgctgcaggagcagggcaccaagactgtgaggcagaacctggagccgttgttcgagcagtacatcaacaacctcaggaggcagctggacagcatcgtgggggaacggggccgcctggactcagagctgagaaacatgcaggacctggtggaagacttcaagaacaagtatgaggatgaaatcaacaagcgtaccactgctgagaatgagtttgtgatgctgaagaaggatgtagatgctgcctacatgaacaaggtggagctggaggccaaggttgatgcactgatggatgagattaacttcatgaagatgttctttgatgcggagctgtcccagatgcagacgcatgtctctgacacctcagtggtcctctccatggacaacaaccgcaacctggacctggatagcatcatcgctgaggtcaaggcccagtatgaggagattgccaaccgcagccggacagaagccgagtcctggtatcagaccaagtatgaggagctgcagcagacagctggccggcatggcgatgacctccgcaacaccaagcatgagatctctgagatgaaccggatgatccagaggctgagagccgagattgacaatgtcaagaaacagtgcgccaatctgcagaacgccattgcggatgccgagcagcgtggggagctggccctcaaggatgccaggaacaagctggccgagctggaggaggccctgcagaaggccaagcaggacatggcccggctgctgcgtgagtaccaggagctcatgaacaccaagctggccctggacgtggagatcgccacttaccgcaagctgctggagggcgaggaatgcagactcagtggagaaggagttggaccagtcaacatctctgttgtcacaagcagtgtttcctctggatatggcagtggcagtggctatggcggtggcctcggtggaggtcttggcggcggcctcggtggaggtcttgccggaggtagcagtggaagctactactccagcagcagtgggggtgtcggcctaggtggtgggctcagtgtggggggctctggcttcagtgcaagcagtggccgagggctgggggtgggctttggcagtggcgggggtagcagctccagcgtcaaatttgtctccaccacctcctcctcccggaagagcttcaagagctaagaacctgctgcaagtcactgccttccaagtgcagcaacccagcccatggagattgcctcttctaggcagttgctcaagccatgttttatccttttctggagagtagtctagaccaagccaattgcagaaccacattctttggttcccaggagagccccattcccagcccctggtctcccgtgccgcagttctatattctgcttcaaatcagccttcaggtttcccacagcatggcccctgctgacacgagaacccaaagttttcccaaatctaaatcatcaaaacagaatccccaccccaatcccaaattttgttttggttctaactacctccagaatgtgttcaataaaatgcttttataatataaaaaaaaaaaaaaaaaaa

SEQ ID NO: 35 - DNA, 1240 bp, Homo sapiens
atgacaccgactcttttgctcacggtgactgtcccgagggcggcgggtagcgccgggcagcgccgggctccagggctcccgcgctccagtggcccagcctgggcggagagcagagcgcggcccccgcggccccgcggcctcgagccccggcacccccctggctccccggccctgcgccccaccgaccgcacgtgctcctcctcctcggcgggagtaggcggcggggtcggaggagcgcagccggggtcggtcccgctgggccagcacctcgctcttgagcggggacgaactctcggacacgggcgtgtcggccggcgagatccgcctccgcttggcctgctcgtaaatccccgagtcgctggagtcgatggacttgatcgaggagggcgtctcgatccagctggaatcggacaggtccttgggcttggcgtcctcggcggcgccgggcggcagcggcgagcgctcggcggccaggccctcggcctcctcgcccaggtagggattggcgccggccatgcgcgcggcggccgcggcgctgttgggccagcagggcagcaccgagcccgacttggtgccgcagtactgcgggggactgcgggcgccccagcccgacgggtcggcgtagtagccgagcgggcggccagtgcagcctgcagcctgcagcggcagcgccttcacgcccgccgccgcgtaagagagcagcgtggccgcgttgcccgcgaagtccgtggccgtgtcataggccgaggccgcgaagtccagccggttgttggccggcgtcacaaaccagcgttgcggcgagggcgcgcccgggtcctcggcctgctgcggcgacagcagcccgttggtgtgcggcacgctgcggtccgtacccggcccggggcccgcgcccgcgcccgggtggaagcgggccttggcgtagttgctcacgaactggtcctgcaggaaagagccggccatggcgtagcgggccccgggcacgatctgcgagcgcggcgagtcgttgggcgagggggtcaggcggtccatgtcacagccggtgtagatcctgcgggattgtttcagaaacccctaggaaaaagccgcgcacgcagtaatcatgaaagactggccttcacccgtgttctggaacccgaggtttgctgctggaagcctccaaagtacttagtgtctattgtttcccctgtgtgaaactttcactcccacctctactaatacaaacaagaattactactctgaa

SEQ ID NO: 36 - DNA, 1634 bp, Homo sapiens
acccgagcaccttctcttcactcagccaactgctcgctcgctcacctccctcctctgcaccatgactacctgcagccgccagttcacctcctccagctccatgaagggctcctgcggcatcgggggcggcatcgggggcggctccagccgcatctcctccgtcctggccggagggtcctgccgcgcccccagcacctacgggggcggcctgtctgtctcatcctcccgcttctcctctgggggagcctacgggctggggggcggctatggcggtggcttcagcagcagcagcagcagctttggtagtggctttgggggaggatatggtggtggccttggtgctggcttgggtggtggctttggtggtggctttgctggtggtgatgggcttctggtgggcagtgagaaggtgaccatgcagaacctcaatgaccgcctggcctcctacctggacaaggtgcgtgctctggaggaggccaacgccgacctggaagtgaagatccgtgactggtaccagaggcagcggcctgctgagatcaaagactacagtccctacttcaagaccattgaggacctgaggaacaagattctcacagccacagtggacaatgccaatgtccttctgcagattgacaatgcccgtctggccgcggatgacttccgcaccaagtatgagacagagttgaacctgcgcatgagtgtggaagccgacatcaatggcctgcgcagggtgctggacgaactgaccctggccagagctgacctggagatgcagattgagagcctgaaggaggagctggcctacctgaagaagaaccacgaggaggagatgaatgccctgagaggccaggtgggtggagatgtcaatgtggagatggacgctgcacctggcgtggacctgagccgcattctgaacgagatgcgtgaccagtatgagaagatggcagagaagaaccgcaaggatgccgaggaatggttcttcaccaagacagaggagctgaaccgcgaggtggccaccaacagcgagctggtgcagagcggcaagagcgagatctcggagctccggcgcaccatgcagaacctggagattgagctgcagtcccagctcagcatgaaagcatccctggagaacagcctggaggagaccaaaggtcgctactgcatgcagctggcccagatccaggagatgattggcagcgtggaggagcagctggcccagctccgctgcgagatggagcagcagaaccaggagtacaagatcctgctggacgtgaagacgcggctggagcaggagatcgccacctaccgccgcctgctggagggcgaggacgcccacctctcctcctcccagttctcctctggatcgcagtcatccagagatgtgacctcctccagccgccaaatccgcaccaaggtcatggatgtgcacgatggcaaggtggtgtccacccacgagcaggtccttcgcaccaagaactgaggctgcccagccccgctcaggcctaggaggccccccgtgtggacacagatcccactggaagatcccctctcctgcccaagcacttcacagctggaccctgcttcaccctcaccccctcctggcaatcaatacagcttcattatctgagttgcat

SEQ ID NO: 37 - DNA, 2946 bp, Homo sapiens
ggaaagccggctcaccttcgcctccccctgcggctgggaggagaggaaatatcccatggctgactgtgccaaggaggtgtctgagccagccctcccggcccgagggcagggcaggtggccctgagagataagccaatcccgcagctgcagatgaggagttctgagaagcattgctcaggacagcggtaaatcacttcttggaggtgccctgcacgccggtcctgggagcaggcggcctcccgggggtgcgggagccccactcctccgtggtgtgttccatttgcttcccacatctggaggagctgacgtgccagcctcccccagcaccacccagggacgggaggcatgagccggtcaaggcacctgggcaaaatccggaagcgtctggaagatgtcaagagccagtgggtccggccagccagggctgactttagtgacaacgagagtgcccggctggccacggacgccctcttggatgggggttctgaagcctactggcgggtgctcagccaggaaggcgaggtggacttcttgtcctcggtggaggcccagtacatccaggcccaggccagggagcccccgtgtcccccagacaccctgggaggggcggaagcaggccctaagggactggactccagctccctacagtccggcacctacttccctgtggcctcagagggcagcgagccggccctactgcacagctgggcctcagctgagaagccctacctgaaggaaaaatccagcgccactgtgtacttccagaccgtcaagcacaacaacatcagagacctcgtccgccgctgcatcacccggactagccaggtcctggtcatcctgatggatgtgttcacggatgtggagatcttctgtgacattctagaggcagccaacaagcgtggggtgttcgtttgtgtgctcctggaccagggaggtgtgaagctcttccaggagatgtgtgacaaagtccagatctctgacagtcacctcaagaacatttccatccggagtgtggaaggagagatatactgtgccaagtcaggcaggaaattcgctggccaaatccgggagaagttcatcatctcggactggagatttgtcctgtctggatcttacagcttcacctggctctgcggacacgtgcaccggaacatcctctccaagttcacaggccaggcggtggagctgtttgacgaggagttccgccacctctacgcctcctccaagcctgtgatgggcctgaagtccccgcggctggtcgcccccgtcccgcccggagcagccccggccaatggccgccttagcagcagcagtggctccgccagtgaccgcacgtcctccaaccccttcagcggccgctcggcaggcagccaccccggtacccgaagtgtgtccgcgtcttcagggccctgtagccccgcggccccacacccgcctccaccgccccggttccagccccaccaaggcccttggggagccccgagtccccaggcccacctctccccgcggccccacgacggcccgcccgccgctgtctacagcaacctgggggcctacaggcccacgcggctgcagctggagcagctgggcctggtgccgaggctgactccaacctggaggcccttcctgcaggcctcccctcacttctgaaggtcccatcccctgctgccctccgcaggcccagggctgggcactccctgagacccaaagacccacctcaacgacgagtggcgttgagccacttccctttgaaaagacactcaaaatcactgccatggttcaatgttcccaggccccaggccatccacttgccggcccccaccagttcttgggttccccgctctagtttgacctgtgcagcacattccagaaggttccagggaggttgtggggcagctagaggacaaaatcatgaaaacagagtccctgtcttccagagatcatccggggctttaatattaatggcccccaaaactccgtaagaagcaggaaatgcagcccaagttttacaaatgggtaaacagaggcactgagagatagatggtagtttggtacttctggttcccagtgcccaggaatggtccactcccaagaaattcaggaaagaaagactgaggagaaggtgtgggaacattctggatgtttcgggagagttggggaaactcctcctcttaggaaaggctaatactagggtatccttgggcccaatgaattaggggtgaggccccagaacccgttatctatgagttgtatgggggagccatctgaagctgtagccaccagggatgcagctagctgaggagtttggggtgttgggttggacaaggcaggttagtagactcagattcttgcttcaaagagccttgggctggcctggaggtccctggagtctagactggacctaggagcttgagttgtcaggggccaggactggccccactgcagtgcccaggccagtcttgagcagcagggagggctcagctgtccccagatccaggtgcctctgaccagcctggtcacctcctgaggaataaatgctgaacctcacaagccccatcattcatttcttctcaattcacagtgcccctctttgtttctggggtggaactaggtcctgagggcacagcctagctgagtgcaaagaaatataggatgcttagaaagcatacaggaggggccaggcgtggtggctcatgcctgtaatcccagaactttgggatgccaaggtggttggattacctgagatcaggtggattacctggtctcgagaccagcctgaccaatatggtgaaaccccgtctctactaaaaatacaaaaattaggctgagacaggagaattgcttgaacccaggaagcagaggttgcaatgagctgagattgcatcactgcactccagcatgggcaacaaagcaagactccgtcacagaaaaaaaaaaaaaaaaaaa

SEQ ID NO: 38 - DNA, 1875 bp, Homo sapiens
agcgcagcgagccaggcccggaactagtaggctgcgccgcgcgcgccgcgccggggcgggagctgggtctgggcggcgggcaggagctggcgggggcgcacgggcagcgctgcggacagcccgggagccgcggcgatggcggtgcaggcggcgctcctcagcacgcaccctttcgtgcccttcggcttcgggggctccccggacgggctagggggcgccttcggagccctggacaagggctgctgtttcgaggacgatgagaccggggctccggcgggtgcgctgctgtcgggagccgaaggaggggacgtgcgcgaggccacccgcgatctactcagcttcattgactcggcgtccagcaacatcaagctggcgctggacaagccgggcaagtcgaagcggaaggtgaaccaccgcaagtacctgcagaagcagatcaagcgctgcagcggcctcatgggcgccgcgccccccggcccgccctccccgagcgccgccgacacgccagccaaacggccgctggccgcccctagcgccccgacagtcgcggccccggcccacggcaaggctgccccccggcgggaggcgtcgcaggccgccgcggccgccagcttgcaaagccgaagtctggccgcgctcttcgactcgctgcgccacgtccccgggggtgccgagccggcggggggtgaggtggctgcgccggcggccgggctaggaggtgcgggcactgggggcgcgggaggggacgtggcaggccccgcgggggccacggcgatcccaggggccaggaaggtcccgctgcgggcacgcaatctgcctccgtccttcttcacggagccgtcccgggcaggcggcggcgggtgtggcccgtcggggccggacgtgagcttgggcgacctggagaagggcgcggaggccgtggagttctttgagctgctggggcccgactacggcgccggcacggaggcggcagtcttgcttgccgccgagcctctcgacgtgttccccgccggagcctccgtactgcggggacccccggagctggagcccggcctctttgagccgccgccggcagtggtgggaaacctactgtaccccgagccctggagcgtcccgggctgctccccgaccaaaaagagccccctgactgccccccgcggcggcttgaccttgaacgagcccttgagccccctgtaccccgccgctgcggattctcccggcggggaggacgggcggggccatttggcctctttcgcccccttctttccagactgcgccctgcccccgccgccgccgccccatcaggtgtcctacgattacagcgcgggctacagccgcaccgcctattccagcctttggagatccgacggggtttgggaaggggcgccgggggaggagggggcgcaccgggactgacttcgaggcacgcttcccttcattagagacggctgtggagagcgccgcgcctccgtgggtttctcctaaatctgaagaacgatgggaaaatgcacgtggagatgaaaccagatttttaaaaattcaattaataaaagcaacttcagaaaaaagagatgaagacgagttggggattgtttaatcacaacctcaagtgttaaaacaaaaacaaacaaacacgtttgtaggttcttactggaccagaggagtcaagaaaccaagatggtttggggtatggggtggggacggcaaaaggggtaagagctggcttctgtagccacctgtcccttctatttttcagcgaaggtcagtgtatttagtgtaattaccccttctaaacagtgtcctagtccctcccttccctctccttgagtgcattttgaattaaagcctatattgaaaagaaaaaaaaaaaaaaaa

SEQ ID NO: 39 - DNA, 617 bp, Homo sapiens
  misc_feature (13)..(13): n is a, c, g, or t
tttccgaggcgtncctnctncccttttnacctcgggactcancgtcttcctcacagcacttccatgtcatctgccccgtgaaatcagcctaacgccgtttctcaatgacgtggatcgccctaggccaccgcaaccttccggaagctctctcagctcagttcccattctcccaccatctcttggttctccttctacctcaccggttgctagtcctccgcttcgcagctgaaaatgtgcccggggcctactgtgggcctagccaggcctgcttacgcagtgcggtttcccatgaatgatgcccagtcattatcacataacctgtggcaagccagcaagatggccctggtgacagcaaaagaaactgcactaggacctgaatgtagatctcagtcatgttccttactaacagcacgttttgcaaccatgcgttaaagaaacatctgactcacaacaaaattttaaagggtttatttgagtgaaaagcaatttatgaattggggaacacctgactgaaagagcgttagtattccagagacaaaacatcaagtgcaagtttttattgggaaaatgtagaagcacaataaagaaattattttgattggttaaaaaaaaaaaaa

SEQ ID NO: 40 - DNA, 955 bp, Homo sapiens
ccaagagctaaaagagagtaagggggaaacaacagcagctccaaccagggcagccttcctgagaagatgcaaccaatcctgcttctgctggccttcctcctgctgcccagggcagatgcaggggagatcatcgggggacatgaggccaagccccactcccgcccctacatggcttatcttatgatctgggatcagaagtctctgaagaggtgcggtggcttcctgatacaagacgacttcgtgctgacagctgctcactgttggggaagctccataaatgtcaccttgggggcccacaatatcaaagaacaggagccgacccagcagtttatccctgtgaaaagacccatcccccatccagcctataatcctaagaacttctccaacgacatcatgctactgcagctggagagaaaggccaagcggaccagagctgtgcagcccctcaggctacctagcaacaaggcccaggtgaagccagggcagacatgcagtgtggccggctgggggcagacggcccccctgggaaaacactcacacacactacaagaggtgaagatgacagtgcaggaagatcgaaagtgcgaatctgacttacgccattattacgacagtaccattgagttgtgcgtgggggacccagagattaaaaagacttcctttaagggggactctggaggccctcttgtgtgtaacaaggtggcccagggcattgtctcctatggacgaaacaatggcatgcctccacgagcctgcaccaaagtctcaagctttgtacactggataaagaaaaccatgaaacgctactaactacaggaagcaaactaagcccccgctgtaatgaaacaccttctctggagccaagtccagatttacactgggagaggtgccagcaactgaataaatacctcttagctgagtggaaaaaaaaaaaaaaaaaaaaaaaaaaaaaaaa

SEQ ID NO: 41 - DNA, 5560 bp, Homo sapiens
aaagcagcagagacgctgcagagggcttttcttagacatcaactgcagacggctggcaggatagaagcagcggctcacttggactttttcaccagggaaatcagagacaatgatggggctcttccccagaactacaggggctctggccatcttcgtggtggtcatattggttcatggagaattgcgaatagagactaaaggtcaatatgatgaagaagagatgactatgcaacaagctaaaagaaggcaaaaacgtgaatgggtgaaatttgccaaaccctgcagagaaggagaagataactcaaaaagaaacccaattgccaagattacttcagattaccaagcaacccagaaaatcacctaccgaatctctggagtgggaatcgatcagccgccttttggaatctttgttgttgacaaaaacactggagatattaacataacagctatagtcgaccgggaggaaactccaagcttcctgatcacatgtcgggctctaaatgcccaaggactagatgtagagaaaccacttatactaacggttaaaattttggatattaatgataatcctccagtattttcacaacaaattttcatgggtgaaattgaagaaaatagtgcctcaaactcactggtgatgatactaaatgccacagatgcagatgaaccaaaccacttgaattctaaaattgccttcaaaattgtctctcaggaaccagcaggcacacccatgttcctcctaagcagaaacactggggaagtccgtactttgaccaattctcttgaccgagagcaagctagcagctatcgtctggttgtgagtggtgcagacaaagatggagaaggactatcaactcaatgtgaatgtaatattaaagtgaaagatgtcaacgataacttcccaatgtttagagactctcagtattcagcacgtattgaagaaaatattttaagttctgaattacttcgatttcaagtaacagatttggatgaagagtacacagataattggcttgcagtatatttctttacctctgggaatgaaggaaattggtttgaaatacaaactgatcctagaactaatgaaggcatcctgaaagtggtgaaggctctagattatgaacaactacaaagcgtgaaacttagtattgctgtcaaaaacaaagctgaatttcaccaatcagttatctctcgataccgagttcagtcaaccccagtcacaattcaggtaataaatgtaagagaaggaattgcattccgtcctgcttccaagacatttactgtgcaaaaaggcataagtagcaaaaaattggtggattatatcctgggaacatatcaagccatcgatgaggacactaacaaagctgcctcaaatgtcaaatatgtcatgggacgtaacgatggtggatacctaatgattgattcaaaaactgctgaaatcaaatttgtcaaaaatatgaaccgagattctactttcatagttaacaaaacaatcacagctgaggttctggccatagatgaatacacgggtaaaacttctacaggcacggtatatgttagagtacccgatttcaatgacaattgtccaacagctgtcctcgaaaaagatgcagtttgcagttcttcaccttccgtggttgtctccgctagaacactgaataatagatacactggcccctatacatttgcactggaagatcaacctgtaaagttgcctgccgtatggagtatcacaaccctcaatgctacctcggccctcctcagagcccaggaacagatacctcctggagtataccacatctccctggtacttacagacagtcagaacaatcggtgtgagatgccacgcagcttgacactggaagtctgtcagtgtgacaacaggggcatctgtggaacttcttacccaaccacaagccctgggaccaggtatggcaggccgcactcagggaggctggggcctgccgccatcggcctgctgctccttggtctcctgctgctgctgttggccccccttctgctgttgacctgtgactgtggggcaggttctactgggggagtgacaggtggttttatcccagttcctgatggctcagaaggaacaattcatcagtggggaattgaaggagcccatcctgaagacaaggaaatcacaaatatttgtgtgcctcctgtaacagccaatggagccgatttcatggaaagttctgaagtttgtacaaatacgtatgccagaggcacagcggtggaaggcacttcaggaatggaaatgaccactaagcttggagcagccactgaatctggaggtgctgcaggctttgcaacagggacagtgtcaggagctgcttcaggattcggagcagccactggagttggcatctgttcctcagggcagtctggaaccatgagaacaaggcattccactggaggaaccaataaggactacgctgatggggcgataagcatgaattttctggactcctacttttctcagaaagcatttgcctgtgcggaggaagacgatggccaggaagcaaatgactgcttgttgatctatgataatgaaggcgcagatgccactggttctcctgtgggctccgtgggttgttgcagttttattgctgatgacctggatgacagcttcttggactcacttggacccaaatttaaaaaacttgcagagataagccttggtgttgatggtgaaggcaaagaagttcagccaccctctaaagacagcggttatgggattgaatcctgtggccatcccatagaagtccagcagacaggatttgttaagtgccagactttgtcaggaagtcaaggagcttctgctttgtccacctctgggtctgtccagccagctgtttccatccctgaccctctgcagcatggtaactatttagtaacggagacttactcggcttctggttccctcgtgcaaccttccactgcaggctttgatccacttctcacacaaaatgtgatagtgacagaaagggtgatctgtcccatttccagtgttcctggcaacctagctggcccaacgcagctacgagggtcacatactatgctctgtacagaggatccttgctcccgtctaatatgaccagaatgagctggaataccacactgaccaaatctggatctttggactaaagtattcaaaatagcatagcaaagctcactgtattgggctaataatttggcacttattagcttctctcataaactgatcacgattataaattaaatgtttgggttcataccccaaaagcaatatgttgtcactcctaattctcaagtactattcaaattgtagtaaatcttaaagtttttcaaaaccctaaaatcatattcgccaggaaattttcctaaacattcttaagcttctatttttcccctgccaaaggaaggtgtttatcattttaaaatgcaatgtgatttagtggattaagcaggagcgctggttcttgtctccattgccttttcttatatcattgataatgatgtaagaatcacaaggggccgggcgcggtggctcacgcctgtaatcccagcactttgggaggccgaggcaggtggatcatgaggtcaggagatcgagaccatcctggctaacaaggtgaaaccccgtctctactaaaaatacaaaaaattagccgggcgcagtggcgggcgcctgtagtcccagctactcgggaggctgaggcaggagaatggcatgaacccgggaagcggagcttgcagtgagccgagattgcgccactgcagtccgcagtccggcctgggcgacagagcgagactccgtctcaaaaaaaaaaaaaaaaaaagaatcacaaggtatttgctaaagcattttgagctgcttggaaaaagggaagtagttgcagtagagtttcttccatcttcttggtgctgggaagccatatatgtgtcttttactcaagctaaggggtataagcttatgtgttgaatttgctacatctatatttcacatattctcacaataagagaattttgaaatagaaatatcatagaacatttaagaaagtttagtataaataatattttgtgtgttttaatccctttgaagggatctatccaaagaaaatattttacactgagctccttcctacacgtctcagtaacagatcctgtgttagtctttgaaaatagctcattttttaaatgtcagtgagtagatgtagcatacatatgatgtataatgacgtgtattatgttaacaatgtctgcagattttgtaggaatacaaaacatggccttttttataagcaaaacgggccaatgactagaataacacatagggcaatctgtgaatatgtattataagcagcattccagaaaagtagttggtgaaataattttcaagtcaaaaagggatatggaaagggaattatgagtaacctctattttttaagccttgcttttaaattaaacagctacagccatttaagccttgaggataataaagcttgagagtaataatgttaggttagcaaaggtttagatgtatcacttcatgcatgctaccatgatagtaatgcagctcttcgagtcatttctggtcattcaagatattcacccttttgcccatagaaagcaccctacctcacctgcttactgacattgtcttagctgatcacaagatcattatcagcctccattattccttactgtatataaaatacagagttttatattttcctttcttcgtttttcaccatattcaaaacctaaatttgtttttgcagatggaatgcaaagtaatcaagtgtttgtgctttcacctagaagggtgtggtcctgaaggaaagaggtcccctaaatatcccccaccctggtgctcctccctctccctggtaccctgactaccaggaagtcaggtgctagagcagctggagaagtgcaggcagcctgtgcttccacagatgggggtgctgctgcaacaaggctttcaatgtgcccatcttaggtgggagaagctagatcctgtgcagcagcctggtaagtcctgaggaggttccattgctcttcctgctgctgtcctttgcttctcaacggtggctcgctctacagtctagagcacatgcagctaacttgtgcctctgcttatgcatgagggttaaattaacaaccataaccttcatttgaagttcaaaggtgtattcaggatcctcaaagcattttaaccttgccgcttaaaacccaatttaccgtgaaatgggaattttgctgcattgttaaactgtagtggaaaccatgctatagtaataaaggttatataagagagaaattgaaattaaatgtgtttttaaatttcaaaaaaaaatcaatctttaggatgacttaaaaattgatttgccatgtaaaatgtatctgcattttttacacaaaacttgttttaagcataaaattttaaaactgtactacttgatgtattatacattttgaaccatatgtattaaaccataaacagtataatgttgttataataaaacaggcaataaatttataaataaaagctgaaaaaaaaaaa

SEQ ID NO: 42 - DNA, 1587 bp, Homo sapiens
cgaggggcggacaccggagagacacgggaaaggggtcgggacaggagcacgtggctcagacaccgacgccgggaggccgcagaccccggacgtgtcaggcatccccgcaggcccggagcgatggcagccttgatgaccccgggaaccggggccccacccgcgcctggtgacttctccggggaagggagccagggacttcccgacccttcgccagagcccaagcagctcccggagctgatccgcatgaagcgagacggaggccgcctgagcgaagcggacatcaggggcttcgtggccgctgtggtgaatgggagcgcgcagggcgcacagatcggggccatgctgatggccatccgacttcggggcatggatctggaggagacctcggtgctgacccaggccctggctcagtcgggacagcagctggagtggccagaggcctggcgccagcagcttgtggacaagcattccacagggggtgtgggtgacaaggtcagcctggtcctcgcacctgccctggcggcatgtggctgcaaggtgccaatgatcagcggacgtggtctggggcacacaggaggcaccttggataagctggagtctattcctggattcaatgtcatccagagcccagagcagatgcaagtgctgctggaccaggcgggctgctgtatcgtgggtcagagtgagcagctggttcctgcggacggaatcctatatgcagccagagatgtgacagccaccgtggacagcctgccactcatcacagcctccattctcagtaagaaactcgtggaggggctgtccgctctggtggtggacgttaagttcggaggggccgccgtcttccccaaccaggagcaggcccgggagctggcaaagacgctggttggcgtgggagccagcctagggcttcgggtcgcggcagcgctgaccgccatggacaagcccctgggtcgctgcgtgggccacgccctggaggtggaggaggcgctgctctgcatggacggcgcaggcccgccagacttaagggacctggtcaccacgctcgggggcgccctgctctggctcagcggacacgcggggactcaggcccagggcgctgcccgggtggccgcggcgctggacgacggctcggcccttggccgcttcgagcggatgctggcggcgcagggcgtggatcccggtctggcccgagccctgtgctcgggaagtcccgcagaacgccggcagctgctgcctcgcgcccgggagcaggaggagctgctggcgcccgcagatggcaccgtggagctggtccgggcgctgccgctggcgctggtgctgcacgagctcggggccgggcgcagccgcgctggggagccgctccgcctgggggtgggcgcagagctgctggtcgacgtgggtcagaggctgcgccgtgggaccccctggctccgcgtgcaccgggacggccccgcgctcagcggcccgcagagccgcgccctgcaggaggcgctcgtactctccgaccgcgcgccattcgccgccccctcgcccttcgcagagctcgttctgccgccgcagcaataaagctcctttgccgcgaaa

SEQ ID NO: 43 - DNA, 2447 bp, Homo sapiens
atatttcatacctttctagaaactgggtgtgatctcactgttggtaaagcccagcccttcccaacctgcaagctcaccttccaggactgggcccagcccatgctctccatatataagctgctgccccgagcctgattcctagtcctgcttctcttccctctctcctccagcctctcacactctcctcagctctctcatctcctggaaccatggccagcacatccaccaccatcaggagccacagcagcagccgccggggtttcagtgccaactcagccaggctccctggggtcagccgctctggcttcagcagcgtctccgtgtcccgctccaggggcagtggtggcctgggtggtgcatgtggaggagctggctttggcagccgcagtctgtatggcctggggggctccaagaggatctccattggagggggcagctgtgccatcagtggcggctatggcagcagagccggaggcagctatggctttggtggcgccgggagtggatttggtttcggtggtggagccggcattggctttggtctgggtggtggagccggccttgctggtggctttgggggccctggcttccctgtgtgcccccctggaggcatccaagaggtcaccgtcaaccagagtctcctgactcccctcaacctgcaaatcgatcccaccatccagcgggtgcgggctgaggagcgtgaacagatcaagaccctcaacaacaagtttgcctccttcatcgacaaggtgcggttcctggagcagcagaacaaggttctggaaacaaagtggaccctgctgcaggagcagggcaccaagactgtgaggcagaacctggagccgttgttcgagcagtacatcaacaacctcaggaggcagctggacagcattgtcggggaacggggccgcctggactcagagctcagaggcatgcaggacctggtggaggacttcaagaacaaatatgaggatgaaatcaacaagcgcacagcagcagagaatgaatttgtgactctgaagaaggatgtggatgctgcctacatgaacaaggttgaactgcaagccaaggcagacactctcacagacgagatcaacttcctgagagccttgtatgatgcagagctgtcccagatgcagacccacatctcagacacatctgtggtgctgtccatggacaacaaccgcaacctggacctggacagcatcatcgctgaggtcaaggcccaatatgaggagattgctcagagaagccgggctgaggctgagtcctggtaccagaccaagtacgaggagctgcaggtcacagcaggcagacatggggacgacctgcgcaacaccaagcaggagattgctgagatcaaccgcatgatccagaggctgagatctgagatcgaccacgtcaagaagcagtgcgccaacctgcaggccgccattgctgatgctgagcagcgtggggagatggccctcaaggatgccaagaacaagctggaagggctggaggatgccctgcagaaggccaagcaggacctggcccggctgctgaaggagtaccaggagctgatgaatgtcaagctggccctggacgtggagatcgccacctaccgcaagctgctggagggtgaggagtgcaggctgaatggcgaaggcgttggacaagtcaacatctctgtggtgcagtccaccgtctccagtggctatggcggtgccagtggtgtcggcagtggcttaggcctgggtggaggaagcagctactcctatggcagtggtcttggcgttggaggtggcttcagttccagcagtggcagagccattgggggtggcctcagctctgttggaggcggcagttccaccatcaagtacaccaccacctcctcctccagcaggaagagctataagcactaaagtgcgtctgctagctctcggtcccacagtcctcaggcccctctctggctgcagagccctctcctcaggttgcctttcctctcctggcctccagtctcccctgctgtcccaggtagagctgggtatggatgcttagtgccctcacttcttctctctctctctataccatctgagcacccattgctcaccatcagatcaacctctgattttacatcatgatgtaatcaccactggagcttcactgttactaaattattaatttcttgcctccagtgttctatctctgaggctgagcattataagaaaatgacctctgctccttttcattgcagaaaattgccaggggcttatttcagaacaacttccacttactttccactggctctcaaactctctaacttataagtgttgtgaacccccacccaggcagtatccatgaaagcacaagtgactagtcctatgatgtacaaagcctgtatctctgtgatgatttctgtgctcttcgctgtttgcaattgctaaataaagcagatttataataca

SEQ ID NO: 44 - DNA, 2330 bp, Homo sapiens
cgcctccagcctctcacactctcctaagccctctcatctcctggaaccatggccagcacatccaccaccatcaggagccacagcagcagccgccggggtttcagtgccaactcagccaggctccctggggtcagccgctctggcttcagcagcatctccgtgtcccgctccaggggcagtggtggcctgggtggcgcatgtggaggagctggctttggcagccgcagtctgtatggcctggggggctccaagaggatctccattggagggggcagctgtgccatcagtggcggctatggcagcagagccggaggcagctatggctttggtggcgccgggagtggatttggtttcggtggtggagccggcattggctttggtctgggtggtggagccggccttgctggtggctttgggggccctggcttccctgtgtgcccccctggaggcatccaagaggtcactgtcaaccagagtctcctgactcccctcaacctgcaaattgaccccgccatccagcgggtgcgggccgaggagcgtgagcagatcaagaccctcaacaacaagtttgcctccttcatcgacaaggtgcggttcctagagcagcagaacaaggttctggacaccaagtggaccctgctgcaggagcagggcaccaagactgtgaggcagaacctggagccgttgttcgagcagtacatcaacaacctcaggaggcagctggacaacatcgtgggggaacggggtcgtctggactcggagctgagaaacatgcaggacctggtggaggacctcaagaacaaatatgaggatgaaatcaacaagcgcacagcagcagagaatgaatttgtgactctgaagaaggatgtggatgctgcctacatgaacaaggttgaactgcaagccaaggcagacactcttacagatgagatcaacttcctgagagccttgtatgatgcagagctgtcccagatgcagacccacatctcagacacatccgtggtgctatccatggacaacaaccgcaacctggacctggacagcatcatcgctgaggtcaaggcccaatatgaggagattgctcagaggagcagggctgaggctgagtcctggtaccagacaaagtacgaggagctgcagatcacagcaggcagacatggggacgacctgcgcaacaccaagcaggagattgctgagatcaaccgcatgatccagaggctgagatctgagatcgaccacgtcaagaagcagtgtgccaacctacaggccgccattgctgatgctgagcagcgtggggagatggccctcaaggatgctaagaacaagctggaagggctggaggatgccctgcagaaggccaagcaggacctggcccggctgctgaaggagtaccaggagctgatgaacgtcaagctggccctggatgtggagatcgccacctaccgcaagctgctggagggcgaggagtgcaggctgaatggcgaaggcgttggacaagtcaacatctctgtagtgcagtccaccgtctccagtggctatggcggtgccagcggtgtcggcagtggcttaggcctgggtggaggaagcagctactcctatggcagtggtcttggcgttggaggcggctttagttccagcagcggcagagccactgggggtggcctcagctctgttggaggcggcagttccaccatcaagtacaccaccacctcctcctccagcaggaagagctacaagcactgaagtgctgccgccagctctcagtcccacagctctcaggcccctctctggcagcagagccctctcctcaggttgcttgtcctcccctggcctccagtctcccctgccctcccgggtagagctgggatgccctcacttttcttctcatcaatacctgttccactgagctcctgttgcttaccatcaagtcaacagttatcagcactcagacatgcgaatgtcctttttagttcccgtattattacaggtatctgagtctgccataattctgagaagaaaatgacctatatccccataagaactgaaactcagtctaggtccagctgcagatgaggagtcctctctttaattgctaaccatcctgcccattatagctacactcaggagttctcatctgacaagtcagttgtcctgatcttctcttgcagtgtccctgaatggcaagtgatgtaccttctgatgcagtctgcattcctgcactgctttctctgctctctttgccttcttttgttctgttgaataaagcatattgagaatgtgaaaaaaaaaaaaaaaaaaaaaaaaaaaaaaaaaaaaaaaaaaaaaaaaaa

SEQ ID NO: 45 - DNA, 1180 bp, Homo sapiens
tagttctccctgagtgagacttgcctgcttctctggcccctggtcctgtcctgttctccagcatggtgtgtctgaagctccctggaggctcctgcatgacagcgctgacagtgacactgatggtgctgagctccccactggctttggctggggacacccgaccacgtttcttgtggcagcttaagtttgaatgtcatttcttcaatgggacggagcgggtgcggttgctggaaagatgcatctataaccaagaggagtccgtgcgcttcgacagcgacgtgggggagtaccgggcggtgacggagctggggcggcctgatgccgagtactggaacagccagaaggacctcctggagcagaggcgggccgcggtggacacctactgcagacacaactacggggttggtgagagcttcacagtgcagcggcgagttgagcctaaggtgactgtgtatccttcaaagacccagcccctgcagcaccacaacctcctggtctgctctgtgagtggtttctatccaggcagcattgaagtcaggtggttccggaacggccaggaagagaaggctggggtggtgtccacaggcctgatccagaatggagattggaccttccagaccctggtgatgctggaaacagttcctcggagtggagaggtttacacctgccaagtggagcacccaagtgtgacgagccctctcacagtggaatggagagcacggtctgaatctgcacagagcaagatgctgagtggagtcgggggcttcgtgctgggcctgctcttccttggggccgggctgttcatctacttcaggaatcagaaaggacactctggacttcagccaacaggattcctgagctgaaatgcagatgaccacattcaaggaagaaccttctgtcccagctttgcagaatgaaaagctttcctgcttggcagttattcttccacaagagagggctttctcaggacctggttgctactggttcggcaactgcagaaaatgtcctcccttgtggcttcctcagctcctgcccttggcctgaagtcccagcattgatgacagcgcctcatcttcaacttttgtgctcccctttgcctaaaccgtatggcctcccgtgcatctgtactcaccctgtacgacaaacacattacattattaaatgtttctcaaagatggag

SEQ ID NO: 46 - DNA, 836 bp, Homo sapiens
actcgccacctcctcttccacccctgccaggcccagcagccaccacagcgcctgcttcctcggccctgaaatcatgcccctaggtctcctgtggctgggcctagccctgttgggggctctgcatgcccaggcccaggactccacctcagacctgatcccagccccacctctgagcaaggtccctctgcagcagaacttccaggacaaccaattccaggggaagtggtatgtggtaggcctggcagggaatgcaattctcagagaagacaaagacccgcaaaagatgtatgccaccatctatgagctgaaagaagacaagagctacaatgtcacctccgtcctgtttaggaaaaagaagtgtgactactggatcaggacttttgttccaggttgccagcccggcgagttcacgctgggcaacattaagagttaccctggattaacgagttacctcgtccgagtggtgagcaccaactacaaccagcatgctatggtgttcttcaagaaagtttctcaaaacagggagtacttcaagatcaccctctacgggagaaccaaggagctgacttcggaactaaaggagaacttcatccgcttctccaaatctctgggcctccctgaaaaccacatcgtcttccctgtcccaatcgaccagtgtatcgacggctgagtgcacaggtgccgccagctgccgcaccagcccgaacaccattgagggagctgggagaccctccccacagtgccacccatgcagctgctccccaggccaccccgctgatggagccccaccttgtctgctaaataaacatgtgccctcaggccaaaaaaaaaaaaaa

SEQ ID NO: 47 - DNA, 2576 bp, Homo sapiens
gacttgctccggtttgcagagctaggaggtggcaggctgtgcgctcaaactcaggctgtctaactccacattctgtggggtgagaggatgggtgatggggtgtcttttctggaggagggaggtgctgtgagcctagcgagatggaggtacagtgggtgtgggcctggagcgctgggcccaggcaggggcttctgattaggaagccctggggcaccagttcaggttctcccagagagtagtgtgatgggatccagtaacctgtgccctccagatgacttctgtaggtgtgtttagtgacatgctcaacgggtgcgggaaggatgggcttgtgccaagggccaagcccagagatgtttcagatttttccctttatgcccctgcaaccaagccctgctgctccaggacatataagagacgaaggctgagggctccagcactcaccggcctgggccctgtcacttctctgatagctcccagctcgctctctgcagccatgattgccagacagcagtgtgtccgaggcgggccccggggcttcagctgtggctcggccattgtaggcggtggcaagagaggtgccttcagctcagtctccatgtctggaggtgctggccgatgctcttctgggggatttggcagcagaagcctctacaacctcagggggaacaaaagcatctccatgagtgtggctgggtcacgacaaggtgcctgctttgggggtgctggaggctttggcactggtggctttggtggtggatttgggggctccttcagtggtaagggtggccctggcttccccgtctgccccgctgggggaattcaggaggtcaccatcaaccagagcttgctcacccccctccacgtggagattgaccctgagatccagaaagtccggacggaagagcgcgaacagatcaagctcctcaacaacaagtttgcctccttcatcgacaaggtgcagttcttagagcaacagaataaggtcctggagaccaaatggaacctgctccagcagcagacgaccaccacctccagcaaaaaccttgagcccctctttgagacctacctcagtgtcctgaggaagcagctagataccttgggcaatgacaaagggcgcctgcagtctgagctgaagaccatgcaggacagcgtggaggacttcaagactaagtatgaagaggagatcaacaaacgcacagcagccgagaatgactttgtggtcctaaagaaggacgtggatgctgcctacctgaacaaggtggagttggaggccaaggtggacagtcttaatgacgagatcaacttcctgaaggtcctctatgatgcggagctgtcccagatgcagacccatgtcagcgacacgtccgtggtcctttccatggacaacaaccgcaacctggacctggacagcattattgccgaggtccgtgcccagtacgaggagattgcccagaggagcaaggctgaggctgaagccctgtaccagaccaaggtccagcagctccagatctcggttgaccaacatggtgacaacctgaagaacaccaagagtgaaattgcagagctcaacaggatgatccagaggctgcgggcagagatcgagaacatcaagaagcagtgccagactcttcaggtatccgtggctgatgcagagcagcgaggtgagaatgcccttaaagatgcccacagcaagcgcgtagagctggaggctgccctgcagcaggccaaggaggagctggcacgaatgctgcgtgagtaccaggagctcatgagtgtgaagctggccttggacatcgagatcgccacctaccgcaaactgctggagggcgaggagtacagaatgtctggagaatgccagagtgccgtgagcatctctgtggtcagcggtagcaccagcactggaggcatcagcggaggattaggaagtggctccgggtttggcctgagtagtggctttggctccggctctggaagtggctttgggtttggtggcagtgtctctggcagttccagcagcaagatcatctctaccaccaccctgaacaagagacgatagaggagacgaggtccctgcagctcactgtgtccagctgggcccagcactggtgtctctgtgcttccttcacttcacctccatcctctgtctctggggctcatcttactagtatcccctccactatcccatgggctctctctgccccaggatgatcttctgtgctgggacagggactctgcctcttggagtttggtagctacttcttgatttgggcctggtgacccacctggaatgggaaggatgtcagctgacctctcacctcccatggacagagaagaaaatgaccaggagtgtcatctccagaattattggggtcacatatgtcccttcccagtccaatgccatctcccactagatcctgtattatccatctacatcagaaccaaactacttctccaacacccggcagcacttggccctgcaagcttaggatgagaaccacttagtgtcccattctactcctctcattccctcttatccatctgcaggtgaatcttcaataaaatgcttttgtcattca

SEQ ID NO: 48 - DNA, 1050 bp, Homo sapiens
ggaccgccgcctggttaaaggcgcttatttcccaggcagccgctgcagtcgccacacctttgcccctgctgcgatgaccctgtcgccacttctgctgttcctgccaccgctgctgctgctgctggacgtccccacggcggcggtgcaggcgtcccctctgcaagcgttagacttctttgggaatgggccaccagttaactacaagacaggcaatctatacctgcgggggcccctgaagaagtccaatgcaccgcttgtcaatgtgaccctctactatgaagcactgtgcggtggctgccgagccttcctgatccgggagctcttcccaacatggctgttggtcatggagatcctcaatgtcacgctggtgccctacggaaacgcacaggaacaaaatgtcagtggcaggtgggagttcaagtgccagcatggagaagaggagtgcaaattcaacaaggtggaggcctgcgtgttggatgaacttgacatggagctagccttcctgaccattgtctgcatggaagagtttgaggacatggagagaagtctgccactatgcctgcagctctacgccccagggctgtcgccagacactatcatggagtgtgcaatgggggaccgcggcatgcagctcatgcacgccaacgcccagcggacagatgctctccagccaccacacgagtatgtgccctgggtcaccgtcaatgggaaacccttggaagatcagacccagctccttacccttgtctgccagttgtaccagggcaagaagccggatgtctgcccttcctcaaccagctccctcaggagtgtttgcttcaagtgatggccggtgagctgcggagagctcatggaaggcgagtgggaacccggctgcctgcctttttttctgatccagaccctcggcacctgctacttaccaactggaaaattttatgcatcccatgaagcccagatacacaaaattccaccccatgatcaagaatcctgctccactaagaatggtgctaaagtaaaactagtttaataagcaaaaaaaaaaaaaaaaaa

SEQ ID NO: 49 - DNA, 1864 bp, Homo sapiens
gttccatcctctgccatctactccactgttcagacacctcctaacctccgtcatgacctgtggcttcaactccataggctgtgggttccgccctggaaacttcagctgtgtctctgcctgcgggccccggccaagccgctgctgcatcaccgccgccccctaccgcggcatctcctgctaccgcggcctcaccgggggctttggcagccacagcgtgtgcgggggcttccgcgccggctcctgcggacgcagcttcggctaccgctccgggggcgtgtgcggacccagccccccatgcatcaccaccgtgtcggtcaacgagagcctcctcacgcccctcaacctggagatagaccccaacgcgcagtgcgtgaagcaggaggagaaggagcagatcaagtccctcaacagcagattcgcggccttcatcgacaaggtgcgcttcctggagcagcagaacaagctgctggagacaaagctgcagttctaccaaaaccgcgagtgctgccagagtaacctggagcccctgtttgctggctacatcgagactctgcggcgggaggccgagtgcgtggaggctgacagtgggaggctggcctcagagctcaatcacgtgcaggaggtgctggagggctacaagaagaagtatgaagaagaagtagcacttcgagccacagcagagaacgagtttgtggctctaaagaaggatgtggactgcgcctacctccgcaagtcagacctggaggccaacgtggaggccctgatccaggagattgacttcctgaggcggctgtacgaggaggagatccgcattctccaatcccacatctcagacacctccgtggttgtcaagctggacaacagccgggacctgaacatggactgcatggttgctgagatcaaggcacagtatgatgacattgccacccgtagccgggctgaggccgagtcctggtatcgcagcaagtgtgaggagatgaaggccacagtgatcaggcacggggagaccctgcgccgcaccaaggaggagatcaatgagctgaaccgcatgatccagaggctgacagccgaggtggagaatgccaagtgccagaactccaagctggaggccgcggtggcccagtctgagcagcagggtgaggcggccctcagtgatgcccgctgcaagctggccgagctggagggcgccctgcagaaggccaagcaagacatggcctgcctgatcagggagtaccaggaggtgatgaactccaagctaggcctggatatcgagatcgccacctacaggcgcctgctggagggcgaggagcataggctgtgtgaaggtgttgaagctgtgaatgtctgtgtcagcagctcccggggtggggttgtgtgcggggacctctgcgtgtcgggctcccggccggtgacgggcagcgtctgcagtgccccctgcaacgggaacctggtggtgagcactggtttgtgcaagccctgtggccagctgaacaccacctgtggagggggctcctgcggccaggggaggtattaagtggcccaaaagagagccaggggagccccttctgcctgccagacgtgccactgccccaccaccagctgaaaacagcagcacatcgctggcttttccccttgtgttctgagaatacaccatcggctcattcccaccagcggctcctccccacctttcatcccactggaaaggggtctgtggctggggaatagacccattccttcccctgtctcagccttcagcccctcccggggagaagggccttgcttccctggaagaagcactgtgagactgttccccctgcctctctggcctcttgtctccccttttccaataaacttggggacctgc

SEQ ID NO: 50 - DNA, 5326 bp, Homo sapiens
cagcgcgcggcaggcggcgagctcgggggccgcagaaaatgaaactgaagccgtggtcacgtgacaggacatgtagtatatagcaggctgccagcgactcctgctcttgcttctggatctgcagggcagtcccagcaggacccatggagtgtccttcgtgccagcatgtctccaaggaggaaacccccaagttctgcagccagtgcggagagaggctgcctcctgcagcccccatagcagattctgagaacaataactccacaatggcgtcggcctcggagggtgaaatggagtgtgggcaggagctgaaggaggaagggggcccgtgcttgttcccgggctcagacagttggcaagaaaaccccgaggagccctgttccaaagcctcctggaccgtccaagaaagcaaaaagaagaaaaggaagaagaaaaagaaggggaacaagtccgcttcctcagagctggcttccttgcccctttctcctgccagcccctgtcacctgactttgctttcaaacccgtggcctcaggacacagccctgccccacagccaagcccagcagagtggccccactggccagccgagccagcccccaggcacagccaccacgccactggagggtgacggcctctccgcgcccaccgaggttggcgacagccccctgcaggcccaggctttgggagaggcaggagtggccacaggaagtgaggctcagagcagcccgcaattccaggaccacacggaaggggaggaccaggacgcttccatcccctctgggggcagaggcctgtcccaggaggggaccggtccccccacctctgctggtgaaggccattctaggactgaagatgctgcccaggagctcctgttgcctgagtcaaaaggaggcagctctgagcccgggacagaactgcagaccaccgagcaacaggcaggggcctcagcctctatggcagttgatgctgtagctgagccagccaatgcagttaaaggggccgggaaggaaatgaaagagaagacccagagaatgaaacagccaccagcaaccactcctcctttcaaaacacactgccaggaagctgagaccaagaccaaggacgagatggctgctgctgaagaaaaagtcggtaagaatgaacaaggggagcctgaagacctcaagaagccagaggggaagaacagaagtgcagctgctgtgaaaaacgagaaggagcaaaaaaaccaggaagcagatgtccaggaagtgaaggcaagcacgctgagcccgggtggaggagtcaccgtgttcttccacgccatcatctctcttcatttcccattcaatcctgacctccataaagtcttcatcagaggaggagaagaatttggggagtcaaaatgggacagcaatatctgtgagctgcactacaccagagacttgggtcatgaccgcgttcttgttgaaggcattgtctgcatttccaagaagcacctagataaatacattccttacaagtacgtcatttataatggggaatcttttgagtatgagttcatttacaagcaccagcagaagaagggcgagtacgtcaaccgctgtctgttcataaaatcttcacttctgggctcaggagactggcatcagtactatgacatagtttatatgaagcctcatgggagactccagaaagtcatgaaccacatcacagacgggccgaggaaggacctggtgaaggggaagcagattgccgctgcgctcatgctggacagcaccttcagcatcctgcagacctgggacaccatcaacctgaacagcttcttcacccagttcgagcagttttgctttgtcctgcaacagcctatgatttatgaaggacaggcacagctgtggaccgatttgcagtacagggagaaagaggtgaagagatacctgtggcaacatctgaaaaaacacgtggtaccattgccggacggaaaaagcacggactttttgcctgtggactgcccagtgaggagtaaactgaaaacaggcctgattgtcctttttgtagtggaaaaaattgagcttttattagaaggcagcctggactggttgtgtcacctcctaacctcagatgccagctcaccagatgagtttcaccgtgacctaagccacatccttgggatacctcagagctggcggctgtacctggtgaacctgtgccaaagatgcatggacacaaggacgtacacctggctgggcgccctgcctgtcctgcactgctgtatggagctggccccgcggcacaaggatgcctggagacagcctgaggacacctgggccgctctggagggactctccttctcaccgttccgggaacaaatgctagatacgagttccctacttcagtttatgagagagaagcagcatttgctgagcatagacgagcctctcttccggtcctggtttagtctgctacctctgagtcacctggttatgtatatggaaaacttcattgagcacctgggtcgttttcctgctcatatcctggactgtctttcagggatttactaccggcttccgggacttgagcaagtcttgaatacgcaggatgttcaggatgttcagaacgttcagaacattttagaaatgctgttgcgactcctggacacttaccgggacaagattcccgaggaggccttgtcaccatcctacctgactgtgtgtctgaaactgcatgaagccatctgcagcagcacaaagctacttaagttttacgagctgccagccttatctgccgagattgtctgcagaatgattagacttctatctctggtggattctgcaggacagagagatgaaactggaaataattcagtccaaacagtcttccaagggacccttgctgctacgaaaaggtggctccgagaagtttttacaaagaacatgctcacatcttcaggtgcctcattcacatacgtcaaggaaattgaggtctggaggcggctggtggaaatccaattccccgcggagcatggctggaaggagtcgttgctgggagacatggaatggaggctcacaaaggaggaacccctctcccagatcactgcctactgcaatagttgctgggacaccaaaggcttagaggacagtgtggccaagaccttcgagaaatgcatcattgaagccgtgagctcagcctgccaggtgaacaatctctcctcctgggaaacggattcgggctcacagctgtgttctgccatgacccagctaagggctatgaagcacccgctgggtctcagctcctccgctaactcagagattgggaagtgggcaccctcctccctcgccaagggcaatggcgctgaaatctagttctctccggattcctcagtgtgctgcacagtccctgctgctcgcaccatcctgcatgtgttccatatggaatcacggccgtgcgcgtgtggcacaagtcacacgggcttgcaggccgttcctcagatggccctgtcatcactgtggctgctggtttgattgattgttaacacttgctcagtaggtgtgcgggaagagactccaaaggttgacagaacatttatggaagcaaaatatgtgaaatggaaaattgtatcaatttatttagctcttttcggcaaaggggaagagattgtgcccccctgtctcccaggaacagtctcgcaggcaatgccacatgaggaagctccctgctggccacggctgccctgctcacatttcctaattggacacttaacccctgtacaagcacagccttgcggccacaggggaagtccagaaacattgaggtcattgaattccggggaccaaggggttctaattttttaagtgactgatacctttgataaggttttcctttccctttttcgttaactctttgttgagatattgttcgtatgccatacggtacatctgtcaaaagttccagttcagcaggttttggggtagtcacagatatgtacagtcatcaccacagttaatgacagagcattttcatcacttcaaagagaaacccggcccctttagccatcatcctcctcccctctagtcacccactcccctctgcaggcataaacaattgctgaacataaacaactgcttctgtggctttctctgttctgactgtcatatgaatggaatcatatcatatgtggccttttgggtatggcttatttcactgagcataatgtttttttgttgttggtggtggtggttgtttgttgtttttgagacagagtttcactctttttgcccaggctggagtgcaatggtgcgatcctggctcaccgcaaccgctgcctcccgggttcaagtgattctcctgcctcagcctcccaagtgctggaattacagctactttttgtttaaagagtcttttaattgtttaaagaacaatgtgccgccacactcggttccttttgtatttttagaagagacagggtttctccatgttggtcaggctggtctcgaactcccgacctcaggtgatccacccaccttggcctcccgaagtgccgagattacaggtgtgagccaccgcgcccggccgagcataatgttttgaaagaccgctcaggctggacacggttgctcacgcctgtaatcacggcactttgggagcccaggagttcaagacaagcctgggtaacagagtaagaccctgtctctataaaaactaaaaaataaacaaaaaaaattagccaggcatggccgggcacacctgtggtcccagctacttgggaggctgaggtgggaggctcccttgggcccagaaggtcaaggcagcagtgagccatgatcacaccactgcacttcaacctgggggacagagcaagaccctgtctcaaaaagcaataacaacaaaagtccatccatattgtagcttgtgtccgtttacgttaagtattccttacccaaaatgcttgagaccagaagtgttttggatttcagatgttttcaaattttggaatatttgcatttacataatgagatgtcttccagatgggacccagagtctaaccacaaaattcacttgtttcatatacatcttatacacatagcctgaaggtaattttatacaatatttttaacaattttgtgcatgagacaaagtttgtattaagtatttgtatgttgaatttttcacttgtggcatcattatactcaaaaagtttggattttgtagtattttggatttggggattaggaatgctcaacctatatttcatttttttccatggccaaatattccccgtttatccatgtgtccattgacggccatctatgttgcttcttcggctattataaatctgctgatacaaaaaaaaaaa

SEQ ID NO: 51 - DNA, 428 bp, Homo sapiens
atgtctcttgtcagctgtctttcagaagacctggtggggcaagtccgtgggcatcatgttgaccgagctggagaaagccttgaactctatcatcgacgtctaccacaagtactccctgataaaggggaatttccatgccgtctacagggatgacctgaagaaattgctagagaccgagtgtcctcagtatatcaggaaaaagggtgcagacgtctggttcaaagagttggatatcaacactgatggtgcagttaacttccaggagttcctcattctggtgataaagatgggcgtggcagcccacaaaaaaagccatgaagaaagccacaaagagtagctgagttactgggcccagaggctgggcccctggacatgtacctgcagaataataaagtcatcaatacctcaaaaaaaaaaaaaaaaaaaaa

SEQ ID NO: 52 - DNA, 1147 bp, Homo sapiens
accaaatcaaccataggtccaagaacaattgtctctggacggcagctatgcgactcaccgtgctgtgtgctgtgtgcctgctgcctggcagcctggccctgccgctgcctcaggaggcgggaggcatgagtgagctacagtgggaacaggctcaggactatctcaagagattttatctctatgactcagaaacaaaaaatgccaacagtttagaagccaaactcaaggagatgcaaaaattctttggcctacctataactggaatgttaaactcccgcgtcatagaaataatgcagaagcccagatgtggagtgccagatgttgcagaatactcactatttccaaatagcccaaaatggacttccaaagtggtcacctacaggatcgtatcatatactcgagacttaccgcatattacagtggatcgattagtgtcaaaggctttaaacatgtggggcaaagagatccccctgcatttcaggaaagttgtatggggaactgctgacatcatgattggctttgcgcgaggagctcatggggactcctacccatttgatgggccaggaaacacgctggctcatgcctttgcgcctgggacaggtctcggaggagatgctcacttcgatgaggatgaacgctggacggatggtagcagtctagggattaacttcctgtatgctgcaactcatgaacttggccattctttgggtatgggacattcctctgatcctaatgcagtgatgtatccaacctatggaaatggagatccccaaaattttaaactttcccaggatgatattaaaggcattcagaaactatatggaaagagaagtaattcaagaaagaaatagaaacttcaggcagaacatccattcattcattcattggattgtatatcattgttgcacaatcagaattgataagcactgttcctccactccatttagcaattatgtcacccttttttattgcagttggtttttgaatgtctttcactccttttaaggataaactcctttatggtgtgactgtgtcttattcatctatacttgcagtgggtagatgtcaataaatgttacatacacaaataaataaaatgtttattccatggtaaatttaaaaaaaaaaaaaaaaaaaaaaaaaaaaa

SEQ ID NO: 53 - DNA, 690 bp, Homo sapiens
aaacccctggtacctgagcactgatctgccttggagaacctgatcctgagactccagcaggatgtcttatcaacagcagcagtgcaagcagccctgccagccacctcctgtgtgccccacgccaaagtgcccagagccatgtccacccccgaagtgccctgagccctgcccaccaccaaagtgtccacagccctgcccacctcagcagtgccagcagaaatatcctcctgtgacaccttccccaccctgccagtcaaagtatccaccgaagagcaagtaacagcttcagaattcatcaggaccaagaaaggataaggatatttggctcacctcgttccacagctccaccttcatcttctcatcaaagcctaccatggatacacagggagcttctttctccttagccagtaatctgcccatgatgatccctgacagcaaaaagtttcttttctgaggctgccatactgccactgtccaggtggagactgagcaaaggaagtcctgggctgtgccagctcccagagcttcggaagaaagagcagcagctctctccctgggaaccatcagagaattctgttgatgtgttctgtgtctgtctgtcacctggtcacgagcttctaccacctttgcaattgtcacttatctttcactccctgaataaagtatctatgcatataaaaaaaaaaaaaa

SEQ ID NO: 54 - DNA, 2330 bp, Homo sapiens
ggggtgcggttaaaaggcgccacggcgggagacaggtgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcagagcaaaccgcccagagtagaagatggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccacgccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtctggaatttgcatcctgctgaatgtcactgaattgtgttatttgctaattagatattgttctgggaagtcaaaaaagccagtttaacgcattgcccagttgttagattaagaaatagacagcatgagagggatgaggcaacccgtgctcagctgtcaaggctcagtcgctagcatttcccaacacaaagattctgaccttaaatgcaaccatttgaaacccctgtaggcctcaggtgaaactccagatgccacaatggagctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagaggctgtctgttgtattcattgtggtcatagcacctaacaacattgtagcctcaatcgagtgagacagactagaagttcctagtgatggcttatgatagcaaatggcctcatgtcaaatatttagatgtaattttgtgtaagaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaagtctgttgattaaagatgagctttgtctacttcaaaagtttgtttgcttaccccttcagcctccaattttttaagtgaaaatatagctaataacatgtgaaaagaatagaagctaaggtttagataaatattgagcagatctataggaagattgaacctgaatattgccattatgcttgacatggtttccaaaaaatggtactccacatatttcagtgagggtaagtattttcctgttgtcaagaatagcattgtaaaagcattttgtaataataaagaatagctttaatgatatgcttgtaactaaaataattttgtaatgtatcaaatacatttaaaacattaaaatataatctctataataattt

SEQ ID NO: 55 - protein, 266 aa, Homo sapiens
MVCLKLPGGSCMTALTVTLMVLSSPLALAGDTRPRFLWQLKFECHFFNGTERVRLLERCIYNQEESVRFDSDVGEYRAVTELGRPDAEYWNSQKDLLEQRRAAVDTYCRHNYGVGESFTVQRRVEPKVTVYPSKTQPLQHHNLLVCSVSGFYPGSIEVRWFRNGQEEKAGVVSTGLIQNGDWTFQTLVMLETVPRSGEVYTCQVEHPSVTSPLTVEWRARSESAQSKMLSGVGGFVLGLLFLGAGLFIYFRNQKGHSGLQPTGFLS

SEQ ID NO: 56 - protein, 403 aa, Homo sapiens [sequence omitted]

SEQ ID NO: 57 - protein, 505 aa, Homo sapiens
MTCGSGFGGRAFSCISACGPRPGRCCITAAPYRGISCYRGLTGGFGSHSVCGGFRAGSCGRSFGYRSGGVCGPSPPCITTVSVNESLLTPLNLEIDPNAQCVKQEEKEQIKSLNSRFAAFIDKVRFLEQQNKLLETKLQFYQNRECCQSNLEPLFEGYIETLRREAECVEADSGRLASELNHVQEVLEGYKKKYEEEVSLRATAENEFVALKKDVDCAYLRKSDLEANVEALIQEIDFLRRLYEEEIRILQSHISDTSVVVKLDNSRDLNMDCIIAEIKAQYDDIVTRSRAEAESWYRSKCEEMKATVIRHGETLRRTKEEINELNRMIQRLTAEVENAKCQNSKLEAAVAQSEQQGEAALSDARCKLAELEGALQKAKQDMACLIREYQEVMNSKLGLDIEIATYRRLLEGEEQRLCEGIGAVNVCVSSSRGGVVCGDLCVSGSRPVTGSVCSAPCNGNVAVSTGLCAPCGQLNTTCGGGSCGVGSCGISSLGVGSCGSSCRKC

SEQ ID NO: 58 - protein, 125 aa, Homo sapiens
MKKSGVLFLLGIILLVLIGVQGTPVVRKGRCSCISTNQGTIHLQSLKDLKQFAPSPSCEKIEIIATLKNGVQTCLNPDSADVKELIKKWEKQVSQKKKQKNGKKHQKKKVLKVRKSQRSRQKKTT

SEQ ID NO: 59 - protein, 267 aa, Homo sapiens
MRLTVLCAVCLLPGSLALPLPQEAGGMSELQWEQAQDYLKRFYLYDSETKNANSLEAKLKEMQKFFGLPITGMLNSRVIEIMQKPRCGVPDVAEYSLFPNSPKWTSKVVTYRIVSYTRDLPHITVDRLVSKALNMWGKEIPLHFRKVVWGTADIMIGFARGAHGDSYPFDGPGNTLAHAFAPGTGLGGDAHFDEDERWTDGSSLGINFLYAATHELGHSLGMGHSSDPNAVMYPTYGNGDPQNFKLSQDDIKGIQKLYGKRSNSRKK

SEQ ID NO: 60 - protein, 267 aa, Homo sapiens; identical to SEQ ID NO: 59

SEQ ID NO: 61 - protein, 117 aa, Homo sapiens
MRASSFLIVVVFLIAGTLVLEAAVTGVPVKGQDTVKGRVPFNGQDPVKGQVSVKGQDKVKAQEPVKGPVSTKPGSCPIILIRCAMLNPPNRCLKDTDCPGIKKCCEGSCGMACFVPQ

SEQ ID NO: 62 - protein, 114 aa, Homo sapiens
MSLPSSRAARVPGPSGSLCALLALLLLLTPPGPLASAGPVSAVLTELRCTCLRVTLRVNPKTIGKLQVFPAGPQCSKVEVVASLKNGKQVCLDPEAPFLKKVIQKILDSGNKKN

SEQ ID NO: 63 - protein, 354 aa, Homo sapiens
MQGLLFSTLLLAGLAQFCCRVQGTGPLDTTPEGRPGEVSDAPQRKQFCHWPCKCPQQKPRCPPGVSLVRDGCGCCKICAKQPGEICNEADLCDPHKGLYCDYSVDRPRYETGVCAYLVAVGCEFNQVHYHNGQVFQPNPLFSCLCVSGAIGCTPLFIPKLAGSHCSGAKGGKKSDQSNCSLEPLLQQLSTSYKTMPAYRNLPLIWKKKCLVQATKWTPCSRTCGMGISNRVTNENSNCEMRKEKRLCYIQPCDSNILKTIKIPKGKTCQPTFQLSKAEKFVFSGCSSTQSYKPTFCGICLDKRCCIPNKSKMITIQFDCPNEGSFKWKMLWITSCVCQRNCREPGDIFSELKIL

SEQ ID NO: 64 - protein, 343 aa, Homo sapiens
MESKYKEILLLTGLDNITDEELDRFKFFLSDEFNIATGKLHTANRIQVATLMIQNAGAVSAVMKTIRIFQKLNYMLLAKRLQEEKEKVDKQYKSVTKPKPLSQAEMSPAASAAIRNDVAKQRAAPKVSPHVKPEQKQMVAQQESIREGFQKRCLPVMVLKAKKPFTFETQEGKQEMFHATVATEKEFFFVKVFNTLLKDKFIPKRIIIIARYYRHSGFLEVNSASRVLDAESDQKVNVPLNIIRKAGETPKINTLQTQPLGTIVNGLFVVQKVTEKKKNILFDLSDNTGKMEVLGVRNEDTMKCKEGDKVRLTFFTLSKNGEKLQLTSGVHSTIKVIKAKKKT

SEQ ID NO: 65 - protein, 149 aa, Homo sapiens
MADQLTEEQVTEFKEAFSLFDKDGDGCITTRELGTVMRSLGQNPTEAELRDMMSEIDRDGNGTVDFPEFLGMMARKMKDTDNEEEIREAFRVFDKDGNGFVSAAELRHVMTRLGEKLSDEEVDEMIRAADTDGDGQVNYEEFVRVLVSK

SEQ ID NO: 66 - protein, 564 aa, Homo sapiens
MASTSTTIRSHSSSRRGFSANSARLPGVSRSGFSSVSVSRSRGSGGLGGACGGAGFGSRSLYGLGGSKRISIGGGSCAISGGYGSRAGGSYGFGGAGSGFGFGGGAGIGFGLGGGAGLAGGFGGPGFPVCPPGGIQEVTVNQSLLTPLNLQIDPTIQRVRAEEREQIKTLNNKFASFIDKVRFLEQQNKVLETKWTLLQEQGTKTVRQNLEPLFEQYINNLRRQLDSIVGERGRLDSELRGMQDLVEDFKNKYEDEINKRTAAENEFVTLKKDVDAAYMNKVELQAKADTLTDEINFLRALYDAELSQMQTHISDTSVVLSMDNNRNLDLDSIIAEVKAQYEEIAQRSRAEAESWYQTKYEELQVTAGRHGDDLRNTKQEIAEINRMIQRLRSEIDHVKKQCANLQAAIADAEQRGEMALKDAKNKLEGLEDALQKAKQDLARLLKEYQELMNVKLALDVEIATYRKLLEGEECRLNGEGVGQVNISVVQSTVSSGYGGASGVGSGLGLGGGSSYSYGSGLGVGGGFSSSSGRAIGGGLSSVGGGSSTIKYTTTSSSSRKSYKH

SEQ ID NO: 67 - protein, 72 aa, Homo sapiens
MSYQQQQCKQPCQPPPVCPTPKCPEPCPPPKCPEPCPPPKCPQPCPPQQCQQKYPPVTPSPPCQSKYPPKSK

SEQ ID NO: 68 - protein, 165 aa, Homo sapiens
MAPNASCLCVHVRSEEWDLMTFDANPYDSVKKIKEHVRSKTKVPVQDQVLLLGSKILKPRRSLSSYGIDKEKTIHLTLKVVKPSDEELPLFLVESGDEAKRHLLQVRRSSSVAQVKAMIETKTGIIPETQIVTCNGKRLEDGKMMADYGIRKGNLLFLASYCIGG

SEQ ID NO: 69 - protein, 390 aa, Homo sapiens
MNSLSEANTKFMFDLFQQFRKSKENNIFYSPISITSALGMVLLGAKDNTAQQIKKVLHFDQVTENTTGKAATYHVDRSGNVHHQFQKLLTEFNKSTDAYELKIANKLFGEKTYLFLQEYLDAIKKFYQTSVESVDFANAPEESRKKINSWVESQTNEKIKNLIPEGNIGSNTTLVLVNAIYFKGQWEKKFNKEDTKEEKFWPNKNTYKSIQMMRQYTSFHFASLEDVQAKVLEIPYKGKDLSMIVLLPNEIDGLQKLEEKLTAEKLMEWTSLQNMRETRVDLHLPRFKVEESYDLKDTLRTMGMVDIFNGDADLSGMTGSRGLVLSGVLHKAFVEVTEEGAEAAAATAVVGFGSSPTSTNEEFHCNHPFLFFIRQNKTNSILFYGRFSSP

SEQ ID NO: 70 - protein, 129 aa, Homo sapiens
MEGLVFSRLSPEYYDPARAHLRDGEKSCPCGQEGPQGDLLTKTQELGRDYRTCLTIVQKLKKMVDKPTQRSVSNAATRVCRTGRSRWRDVCRNFMRRYQSRVIQGLVAGETAQQICEDLRLCIPSTGPL

SEQ ID NO: 71 - protein, 391 aa, Homo sapiens
MDSLGAVSTRLGFDLFKELKKTNDGNIFFSPVGILTAIGMVLLGTRGATASQLEEVFHSEKETKSSRIKAEEKEVIENTEAVHQQFQKFLTEISKLTNDYELNITNRLFGEKTYLFLQKYLDYVEKYYHASLEPVDFVNAADESRKKINSWVESKTNEKIKDLFPDGSISSSTKLVLVNMVYFKGQWDREFKKENTKEEKFWMNKSTSKSVQMMTQSHSFSFTFLEDLQAKILGIPYKNNDLSMFVLLPNDIDGLEKIIDKISPEKLVEWTSPGHMEERKVNLHLPRFEVEDGYDLEAVLAAMGMGDAFSEHKADYSGMSSGSGLYAQKFLHSSFVAVTEEGTEAAAATGIGFTVTSAPGHENVHCNHPFLFFIRHNESNSILFFGRFSSP

SEQ ID NO: 72 - protein, 369 aa, Homo sapiens
MPRAPKRQRCMPEEDLQSQSETQGLEGAQAPLAVEEDASSSTSTSSSFPSSFPSSSSSSSSSCYPLIPSTPEEVSADDETPNPPQSAQIACSSPSVVASLPLDQSDEGSSSQKEESPSTLQVLPDSESLPRSEIDEKVTDLVQFLLFKYQMKEPITKAEILESVIKNYEDHFPLLFSEASECMLLVFGIDVKEVDPTGHSFVLVTSLGLTYDGMLSDVQSMPKTGILILILSIIFIEGYCTPEEVIWEALNMMGLYDGMEHLIYGEPRKLLTQDWVQENYLEYRQVPGSDPARYEFLWGPRAHAEIRKMSLLKFLAKVNGSDPRSFPLWYEEALKDEEERAQDRIATTDDTTAMASASSSATGSFSYPE

SEQ ID NO: 73 - protein, 1549 aa, Homo sapiens
MSLSFCGNNISSYNINDGVLQNSCFVDALNLVPHVFLLFITFPILFIGWGSQSSKVQIHHNTWLHFPGHNLRWILTFALLFVHVCEIAEGIVSDSRRESRHLHLFMPAVMGFVATTTSIVYYHNIETSNFPKLLLALFLYWVMAFITKTIKLVKYCQSGLDISNLRFCITGMMVILNGLLMAVEINVIRVRRYVFFMNPQKVKPPEDLQDLGVRFLQPFVNLLSKATYWWMNTLIISAHKKPIDLKAIGKLPIAMRAVTNYVCLKDAYEEQKKKVADHPNRTPSIWLAMYRAFGRPILLSSTFRYLADLLGFAGPLCISGIVQRVNETQNGTNNTTGISETLSSKEFLENAYVLAVLLFLALILQRTFLQASYYVTIETGINLRGALLAMIYNKILRLSTSNLSMGEMTLGQINNLVAIETNQLMWFLFLCPNLWAMPVQIIMGVILLYNLLGSSALVGAAVIVLLAPIQYFIATKLAEAQKSTLDYSTERLKKTNEILKGIKLLKLYAWEHIFCKSVEETRMKELSSLKTFALYTSLSIFMNAAIPIAAVLATFVTHAYASGNNLKPAEAFASLSLFHILVTPLSLLFTVVRFAVKAIISVQKLNEFLLSDEIGDDSWRTGESSLPFESCKKHTGVQPKTINRKQPGRYHLDSYEQSTRRLRPAETEDIAIKVTNGYFSWGSGLATLSNIDIRIPTGQLTMIVGQVGCGKSSLLLAILGEMQTLEGKVHWSNVNESEPSFEATRSRNRYSVAYAAQKPWLLNATVEENITFGSPFNKQRYKAVTDACSLQPDIDLLPFGDQTEIGERGINLSGGQRQRICVARALYQNTNIVFLDDPFSALDIHLSDHLMQEGILKFLQDDKRTLVLVTHKLQYLTHADWIIAMKDGSVLREGTLKDIQTKDVELYEHWKTLMNRQDQELEKDMEADQTTLERKTLRRAMYSREAKAQMEDEDEEEEEEEDEDDNMSTVMRLRTKMPWKTCWRYLTSGGFFLLILMIFSKLLKHSVIVAIDYWLATWTSEYSINNTGKADQTYYVAGFSILCGAGIFLCLVTSLTVEWMGLTAAKNLHHNLLNKIILGPIRFFDTTPLGLILNRFSADTNIIDQHIPPTLESLTRSTLLCLSAIGMISYATPVFLVALLPLGVAFYFIQKYFRVASKDLQELDDSTQLPLLCHFSETAEGLTTIRAFRHETRFKQRMLELTDTNNIAYLFLSAANRWLEVRTDYLGACIVLTASIASISGSSNSGLVGLGLLYALTITNYLNWVVRNLADLEVQMGAVKKVNSFLTMESENYEGTMDPSQVPEHWPQEGEIKIHDLCVRYENNLKPVLKHVKAYIKPGQKVGICGRTGSGKSSLSLAFFRMVDIFDGKIVIDGIDISKLPLHTLRSRLSIILQDPILFSGSIRFNLDPECKCTDDRLWEALEIAQLKNMVKSLPGGLDAVVTEGGENFSVGQRQLFCLARAFVRKSSILIMDEATASIDMATENILQKVVMTAFADRTVVTMAHRVHTILTADLVIVMKRGNILEYDTPESLLAQENGVFASFVRADM

SEQ ID NO: 74 - protein, 472 aa, Homo sapiens
MTTCSRQFTSSSSMKGSCGIGGGIGGGSSRISSVLAGGSCRAPSTYGGGLSVSSSRFSSGGAYGLGGGYGGGFSSSSSSFGSGFGGGYGGGLGAGLGGGFGGGFAGGDGLLVGSEKVTMQNLNDRLASYLDKVRALEEANADLEVKIRDWYQRQRPAEIKDYSPYFKTIEDLRNKILTATVDNANVLLQIDNARLAADDFRTKYETELNLRMSVEADINGLRRVLDELTLARADLEMQIESLKEELAYLKKNHEEEMNALRGQVGGDVNVEMDAAPGVDLSRILNEMRDQYEKMAEKNRKDAEEWFFTKTEELNREVATNSELVQSGKSEISELRRTMQNLEIELQSQLSMKASLENSLEETKGRYCMQLAQIQEMIGSVEEQLAQLRCEMEQQNQEYKILLDVKTRLEQEIATYRRLLEGEDAHLSSSQFSSGSQSSRDVTSSSRQIRTKVMDVHDGKVVSTHEQVLRTKN

SEQ ID NO: 75 - protein, 586 aa, Homo sapiens
MALEIHMSDPMCLIENFNEQLKVNQEALEILSAITQPVVVVAIVGLYRTGKSYLMNKLAGKNKGFSVASTVQSHTKGIWIWCVPHPNWPNHTLVLLDTEGLGDVEKADNKNDIQIFALALLLSSTFVYNTVNKIDQGAIDLLHNVTELTDLLKARNSPDLDRVEDPADSASFFPDLVWTLRDFCLGLEIDGQLVTPDEYLENSLRPKQGSDQRVQNFNLPRLCIQKFFPKKKCFIFDLPAHQKKLAQLETLPDDELEPEFVQQVTEFCSYIFSHSMTKTLPGGIMVNGSRLKNLVLTYVNAISSGDLPCIENAVLALAQRENSAAVQKAIAHYDQQMGQKVQLPMETLQELLDLHRTSEREAIEVFMKNSFKDVDQSFQKELETLLDAKQNDICKRNLEASSDYCSALLKDIFGPLEEAVKQGIYSKPGGHNLFIQKTEELKAKYYREPRKGIQAEEVLQKYLKSKESVSHAILQTDQALTETEKKKKEAQVKAEAEKAEAQRLAAIQRQNEQMMQERERLHQEQVRQMEIAKQNWLAEQQKMQEQQMQEQAAQLSTTFQAQNRSLLSELQHAQRTVNNDDPCVLL

SEQ ID NO: 76 - protein, 433 aa, Homo sapiens
MRQSHQLPLVGLLLFSFIPSQLCEICEVSEENYIRLKPLLNTMIQSNYNRGTSAVNVVLSLKLVGIQIQTLMQKMIQQIKYNVKSRLSDVSSGELALIILALGVCRNAEENLIYDYHLIDKLENKFQAEIENMEAHNGTPLTNYYQLSLDVLALCLFNGNYSTAEVVNHFTPENKNYYFGSQFSVDTGAMAVLALTCVKKSLINGQIKADEGSLKNISIYTKSLVEKILSEKKENGLIGNTFSTGEAMQALFVSSDYYNENDWNCQQTLNTVLTEISQGAFSNPNAAAQVLPALMGKTFLDINKDSSCVSASGNFNISADEPITVTPPDSQSYISVNYSVRINETYFTNVTVLNGSVFLSVMEKAQKMNDTIFGFTMEERSWGPYITCIQGLCANNNDRTYWELLSGGEPLSQGAGSYVVRNGENLEVRWSKY

SEQ ID NO: 77 - protein, 191 aa, Homo sapiens
MGKQNSKLAPEVMEDLVKSTEFNEHELKQWYKGFLKDCPSGRLNLEEFQQLYVKFFPYGDASKFAQHAFRTFDKNGDGTIDFREFICALSITSRGSFEQKLNWAFNMYDLDGDGKITRVEMLEIIEAIYKMVGTVIMMKMNEDGLTPEQRVDKIFSKMDKNKDDQITLDEFKEAAKSDPSIVLLLQCDIQK

SEQ ID NO: 78 - protein, 167 aa, Homo sapiens
MEETYTDSLDPEKLLQCPYDKNHQIRACRFPYHLIKCRKNHPDVASKLATCPFNARHQVPRAEISHHISSCDDRSCIEQDVVNQTRSLRQETLAESTWQCPPCDEDWDKDLWEQTSTPFAWGTTHYSDNNSPASNIVTEHKNNLASGMRVPKSLPYVLPWKNNGNAQ

SEQ ID NO: 79 - protein, 93 aa, Homo sapiens
MLTELEKALNSIIDVYHKYSLIKGNFHAVYRDDLKKLLETECPQYIRKKGADVWFKELDINTDGAVNFQEFLILVIKMGVAAHKKSHEESHKE

SEQ ID NO: 80 - protein, 390 aa, Homo sapiens
MNSLSEANTKFMFDLFQQFRKSKENNIFYSPISITSALGMVLLGAKDNTAQQISKVLHFDQVTENTTEKAATYHVDRSGNVHHQFQKLLTEFNKSTDAYELKIANKLFGEKTYQFLQEYLDAIKKFYQTSVESTDFANAPEESRKKINSWVESQTNEKIKNLFPDGTIGNDTTLVLVNAIYFKGQWENKFKKENTKEEKFWPNKNTYKSVQMMRQYNSFNFALLEDVQAKVLEIPYKGKDLSMIVLLPNEIDGLQKLEEKLTAEKLMEWTSLQNMRETCVDLHLPRFKMEESYDLKDTLRTMGMVNIFNGDADLSGMTWSHGLSVSKVLHKAFVEVTEEGVEAAAATAVVVVELSSPSTNEEFCCNHPFLFFIRQNKTNSILFYGRFSSP

SEQ ID NO: 81 - protein, 101 aa, Homo sapiens
MSNTQAERSIIGMIDMFHKYTGRDGKIEKPSLLTMMKENFPNFLSACDKKGIHYLATVFEKKDKNEDKKIDFSEFLSLLGDIAADYHKQSHGAAPCSGGSQ

SEQ ID NO: 82 - protein, 250 aa, Homo sapiens
MTLSPLLLFLPPLLLLLDVPTAAVQASPLQALDFFGNGPPVNYKTGNLYLRGPLKKSNAPLVNVTLYYEALCGGCRAFLIRELFPTWLLVMEILNVTLVPYGNAQEQNVSGRWEFKCQHGEEECKFNKVEACVLDELDMELAFLTIVCMEEFEDMERSLPLCLQLYAPGLSPDTIMECAMGDRGMQLMHANAQRTDALQPPHEYVPWVTVNGKPLEDQTQLLTLVCQLYQGKKPDVCPSSTSSLRSVCFK

SEQ ID NO: 83 - protein, 510 aa, Homo sapiens
MRPGLSFLLALLFFLGQAAGDLGDVGPPIPSPGFSSFPGVDSSSSFSSSSRSGSSSSRSLGSGGSVSQLFSNFTGSVDDRGTCQCSVSLPDTTFPVDRVERLEFTAHVLSQKFEKELSKVREYVQLISVYEKKLLNLTVRIDIMEKDTISYTELDFELIKVEVKEMEKLVIQLKESFGGSSEIVDQLEVEIRNMTLLVEKLETLDKNNVLAIRREIVALKTKLKECEASKDQNTPVVHPPPTPGSCGHGGVVNISKPSVVQLNWRGFSYLYGAWGRDYSPQHPNKGLYWVAPLNTDGRLLEYYRLYNTLDDLLLYINARELRITYGQGSGTAVYNNNMYVNMYNTGNIARVNLTTNTIAVTQTLPNAAYNNRFSYANVAWQDIDFAVDENGLWVIYSTEASTGNMVISKLNDTTLQVLNTWYTKQYKPSASNAFMVCGVLYATRTMNTRTEEIFYYYDTNTGKEGKLDIVMHKMQEKVQSINYNPFDQKLYVYNDGYLLNYDLSVLQKPQ

SEQ ID NO: 84 - protein, 198 aa, Homo sapiens
MPLGLLWLGLALLGALHAQAQDSTSDLIPAPPLSKVPLQQNFQDNQFQGKWYVVGLAGNAILREDKDPQKMYATIYELKEDKSYNVTSVLFRKKKCDYWIRTFVPGCQPGEFTLGNIKSYPGLTSYLVRVVSTNYNQHAMVFFKKVSQNREYFKITLYGRTKELTSELKENFIRFSKSLGLPENHIVFPVPIDQCIDG

SEQ ID NO: 85 - protein, 175 aa, Homo sapiens
MQRRLVQQWSVAVFLLSYAVPSCGRSVEGLSRRLKRAVSEHQLLHDKGKSIQDLRRRFFLHHLIAEIHTAEIRATSEVSPNSKPSPNTKNHPVRFGSDDEGRYLTQETNKVETYKEQPLKTPGKKKKGKPGKRKEQEKKKRRTRSAWLDSGVTGSGLEGDHLSDTSTTSLELDSR

SEQ ID NO: 86 - protein, 261 aa, Homo sapiens
MDWGTLHTFIGGVNKHSTSIGKVWITVIFIFRVMILVVAAQEVWGDEQEDFVCNTLQPGCKNVCYDHFFPVSHIRLWALQLIFVSTPALLVAMHVAYYRHETTRKFRRGEKRNDFKDIEDIKKQKVRIEGSLWWTYTSSIFFRIIFEAAFMYVFYFLYNGYHLPWVLKCGIDPCPNLVDCFISRPTEKTVFTIFMISASVICMLLNVAELCYLLLKVCFRRSKRAQTQKNHPNHALKESKQNEMNELISDSGQNAITGFPS

SEQ ID NO: 87 - protein, 434 aa, Homo sapiens
MSRSRHLGKIRKRLEDVKSQWVRPARADFSDNESARLATDALLDGGSEAYWRVLSQEGEVDFLSSVEAQYIQAQAREPPCPPDTLGGAEAGPKGLDSSSLQSGTYFPVASEGSEPALLHSWASAEKPYLKEKSSATVYFQTVKHNNIRDLVRRCITRTSQVLVILMDVFTDVEIFCDILEAANKRGVFVCVLLDQGGVKLFQEMCDKVQISDSHLKNISIRSVEGEIYCAKSGRKFAGQIREKFIISDWRFVLSGSYSFTWLCGHVHRNILSKFTGQAVELFDEEFRHLYASSKPVMGLKSPRLVAPVPPGAAPANGRLSSSSGSASDRTSSNPFSGRSAGSHPGTRSVSASSGPCSPAAPHPPPPPRFQPHQGPWGAPSPQAHLSPRPHDGPPAAVYSNLGAYRPTRLQLEQLGLVPRLTPTWRPFLQASPHF

SEQ ID NO: 88 - protein, 226 aa, Homo sapiens
MDWGTLQTILGGVNKHSTSIGKIWLTVLFIFRIMILVVAAKEVWGDEQADFVCNTLQPGCKNVCYDHYFPISHIRLWALQLIFVSTPALLVAMHVAYRRHEKKRKFIKGEIKSEFKDIEEIKTQKVRIEGSLWWTYTSSIFFRVIFEAAFMYVFYVMYDGFSMQRLVKCNAWPCPNTVDCFVSRPTEKTVFTVFMIAVSGICILLNVTELCYLLIRYCSGKSKKPV

SEQ ID NO: 89 - protein, 37 aa, Homo sapiens
MKEPGPNFVTVRKGLHSFKMAFVKHLLQTLEIKKVLE

SEQ ID NO: 90 - protein, 27 aa, Homo sapiens
MKEPGPNFVTVRKGLHSFKMAFVKHLL

SEQ ID NO: 91 - protein, 426 aa, Homo sapiens
MAVQAALLSTHPFVPFGFGGSPDGLGGAFGALDKGCCFEDDETGAPAGALLSGAEGGDVREATRDLLSFIDSASSNIKLALDKPGKSKRKVNHRKYLQKQIKRCSGLMGAAPPGPPSPSAADTPAKRPLAAPSAPTVAAPAHGKAAPRREASQAAAAASLQSRSLAALFDSLRHVPGGAEPAGGEVAAPAAGLGGAGTGGAGGDVAGPAGATAIPGARKVPLRARNLPPSFFTEPSRAGGGGCGPSGPDVSLGDLEKGAEAVEFFELLGPDYGAGTEAAVLLAAEPLDVFPAGASVLRGPPELEPGLFEPPPAVVGNLLYPEPWSVPGCSPTKKSPLTAPRGGLTLNEPLSPLYPAAADSPGGEDGRGHLASFAPFFPDCALPPPPPPHQVSYDYSAGYSRTAYSSLWRSDGVWEGAPGEEGAHRD

SEQ ID NO: 92 - protein, 1063 aa, Homo sapiens
MECPSCQHVSKEETPKFCSQCGERLPPAAPIADSENNNSTMASASEGEMECGQELKEEGGPCLFPGSDSWQENPEEPCSKASWTVQESKKKKRKKKKKGNKSASSELASLPLSPASPCHLTLLSNPWPQDTALPHSQAQQSGPTGQPSQPPGTATTPLEGDGLSAPTEVGDSPLQAQALGEAGVATGSEAQSSPQFQDHTEGEDQDASIPSGGRGLSQEGTGPPTSAGEGHSRTEDAAQELLLPESKGGSSEPGTELQTTEQQAGASASMAVDAVAEPANAVKGAGKEMKEKTQRMKQPPATTPPFKTHCQEAETKTKDEMAAAEEKVGKNEQGEPEDLKKPEGKNRSAAAVKNEKEQKNQEADVQEVKASTLSPGGGVTVFFHAIISLHFPFNPDLHKVFIRGGEEFGESKWDSNICELHYTRDLGHDRVLVEGIVCISKKHLDKYIPYKYVIYNGESFEYEFIYKHQQKKGEYVNRCLFIKSSLLGSGDWHQYYDIVYMKPHGRLQKVMNHITDGPRKDLVKGKQIAAALMLDSTFSILQTWDTINLNSFFTQFEQFCFVLQQPMIYEGQAQLWTDLQYREKEVKRYLWQHLKKHVVPLPDGKSTDFLPVDCPVRSKLKTGLIVLFVVEKIELLLEGSLDWLCHLLTSDASSPDEFHRDLSHILGIPQSWRLYLVNLCQRCMDTRTYTWLGALPVLHCCMELAPRHKDAWRQPEDTWAALEGLSFSPFREQMLDTSSLLQFMREKQHLLSIDEPLFRSWFSLLPLSHLVMYMENFIEHLGRFPAHILDCLSGIYYRLPGLEQVLNTQDVQDVQNVQNILEMLLRLLDTYRDKIPEEALSPSYLTVCLKLHEAICSSTKLLKFYELPALSAEIVCRMIRLLSLVDSAGQRDETGNNSVQTVFQGTLAATKRWLREVFTKNMLTSSGASFTYVKEIEVWRRLVEIQFPAEHGWKESLLGDMEWRLTKEEPLSQITAYCNSCWDTKGLEDSVAKTFEKCIIEAVSSACQVNNLSSWETDSGSQLCSAMTQLRAMKHPLGLSSSANSEIGKWAPSSLAKGNGAEI

SEQ ID NO: 93 - protein, 470 aa, Homo sapiens
MKFLLILLLQATASGALPLNSSTSLEKNNVLFGERYLEKFYGLEINKLPVTKMKYSGNLMKEKIQEMQHFLGLKVTGQLDTSTLEMMHAPRCGVPDVHHFREMPGGPVWRKHYITYRINNYTPDMNREDVDYAIRKAFQVWSNVTPLKFSKINTGMADILVVFARGAHGDFHAFDGKGGILAHAFGPGSGIGGDAHFDEDEFWTTHSGGTNLFLTAVHEIGHSLGLGHSSDPKAVMFPTYKYVDINTFRLSADDIRGIQSLYGDPKENQRLPNPDNSEPALCDPNLSFDAVTTVGNKIFFFKDRFFWLKVSERPKTSVNLISSLWPTLPSGIEAAYEIEARNQVFLFKDDKYWLISNLRPEPNYPKSIHSFGFPNFVKKIDAAVFNPRFYRTYFFVDNQYWRYDERRQMMDPGYPKLITKNFQGIGPKIDAVFYSKNKYYYFFQGSNQFEYDFLLQRITKTLKSNSWFGC

SEQ ID NO: 94 - protein, 594 aa, Homo sapiens
MTSVGVFSDMLNGCGKDGLVPRAKPRDVSDFSLYAPATKPCCSRTYKRRRLRAPALTGLGPVTSLIAPSSLSAAMIARQQCVRGGPRGFSCGSAIVGGGKRGAFSSVSMSGGAGRCSSGGFGSRSLYNLRGNKSISMSVAGSRQGACFGGAGGFGTGGFGGGFGGSFSGKGGPGFPVCPAGGIQEVTINQSLLTPLHVEIDPEIQKVRTEEREQIKLLNNKFASFIDKVQFLEQQNKVLETKWNLLQQQTTTTSSKNLEPLFETYLSVLRKQLDTLGNDKGRLQSELKTMQDSVEDFKTKYEEEINKRTAAENDFVVLKKDVDAAYLNKVELEAKVDSLNDEINFLKVLYDAELSQMQTHVSDTSVVLSMDNNRNLDLDSIIAEVRAQYEEIAQRSKAEAEALYQTKVQQLQISVDQHGDNLKNTKSEIAELNRMIQRLRAEIENIKKQCQTLQVSVADAEQRGENALKDAHSKRVELEAALQQAKEELARMLREYQELMSVKLALDIEIATYRKLLEGEEYRMSGECQSAVSISVVSGSTSTGGISGGLGSGSGFGLSSGFGSGSGSGFGFGGSVSGSSSSKIISTTTLNKRR

SEQ ID NO: 95 - protein, 140 aa, Homo sapiens
MASAARRSSGRHTSRPTTPGAAQRRCVLAALRGFRRGPAGLGRETRVPAGAGLGDATAAISHRGGVGKRGSLRLQGLSTASGQPQQRPPVSAGQRARPVPRPPSSSAGPGPEGPEGAGCVLRLSAISAGPELRPDHFLLE

SEQ ID NO: 96 - protein, 101 aa, Homo sapiens
MSNTQAERSIIGMIDMFHKYTRRDDKIEKPSLLTMMKENFPNFLSACDKKGTNYLADVFEKKDKNEDKKIDFSEFLSLLGDIATDYHKQSHGAAPCSGGSQ

SEQ ID NO: 97 - protein, 590 aa, Homo sapiens
MSRQSSVSFRSGGSRSFSTASAITPSVSRTSFTSVSRSGGGGGGGFGRVSLAGACGVGGYGSRSLYNLGGSKRISISTSGGSFRNRFGAGAGGGYGFGGGAGSGFGFGGGAGGGFGLGGGAGFGGGFGGPGFPVCPPGGIQEVTVNQSLLTPLNLQIDPSIQRVRTEEREQIKTLNNKFASFIDKVRFLEQQNKVLDTKWTLLQEQGTKTVRQNLEPLFEQYINNLRRQLDSIVGERGRLDSELRNMQDLVEDFKNKYEDEINKRTTAENEFVMLKKDVDAAYMNKVELEAKVDALMDEINFMKMFFDAELSQMQTHVSDTSVVLSMDNNRNLDLDSIIAEVKAQYEEIANRSRTEAESWYQTKYEELQQTAGRHGDDLRNTKHEISEMNRMIQRLRAEIDNVKKQCANLQNAIADAEQRGELALKDARNKLAELEEALQKAKQDMARLLREYQELMNTKLALDVEIATYRKLLEGEECRLSGEGVGPVNISVVTSSVSSGYGSGSGYGGGLGGGLGGGLGGGLAGGSSGSYYSSSSGGVGLGGGLSVGGSGFSASSGRGLGVGFGSGGGSSSSVKFVSTTSSSRKSFKS

SEQ ID NO: 98 - protein, 564 aa, Homo sapiens
MASTSTTIRSHSSSRRGFSANSARLPGVSRSGFSSISVSRSRGSGGLGGACGGAGFGSRSLYGLGGSKRISIGGGSCAISGGYGSRAGGSYGFGGAGSGFGFGGGAGIGFGLGGGAGLAGGFGGPGFPVCPPGGIQEVTVNQSLLTPLNLQIDPAIQRVRAEEREQIKTLNNKFASFIDKVRFLEQQNKVLDTKWTLLQEQGTKTVRQNLEPLFEQYINNLRRQLDNIVGERGRLDSELRNMQDLVEDLKNKYEDEINKRTAAENEFVTLKKDVDAAYMNKVELQAKADTLTDEINFLRALYDAELSQMQTHISDTSVVLSMDNNRNLDLDSIIAEVKAQYEEIAQRSRAEAESWYQTKYEELQITAGRHGDDLRNTKQEIAEINRMIQRLRSEIDHVKKQCANLQAAIADAEQRGEMALKDAKNKLEGLEDALQKAKQDLARLLKEYQELMNVKLALDVEIATYRKLLEGEECRLNGEGVGQVNISVVQSTVSSGYGGASGVGSGLGLGGGSSYSYGSGLGVGGGFSSSSGRATGGGLSSVGGGSSTIKYTTTSSSSRKSYKH

SEQ ID NO: 99 - protein, 999 aa, Homo sapiens
MMGLFPRTTGALAIFVVVILVHGELRIETKGQYDEEEMTMQQAKRRQKREWVKFAKPCREGEDNSKRNPIAKITSDYQATQKITYRISGVGIDQPPFGIFVVDKNTGDINITAIVDREETPSFLITCRALNAQGLDVEKPLILTVKILDINDNPPVFSQQIFMGEIEENSASNSLVMILNATDADEPNHLNSKIAFKIVSQEPAGTPMFLLSRNTGEVRTLTNSLDREQASSYRLVVSGADKDGEGLSTQCECNIKVKDVNDNFPMFRDSQYSARIEENILSSELLRFQVTDLDEEYTDNWLAVYFFTSGNEGNWFEIQTDPRTNEGILKVVKALDYEQLQSVKLSIAVKNKAEFHQSVISRYRVQSTPVTIQVINVREGIAFRPASKTFTVQKGISSKKLVDYILGTYQAIDEDTNKAASNVKYVMGRNDGGYLMIDSKTAEIKFVKNMNRDSTFIVNKTITAEVLAIDEYTGKTSTGTVYVRVPDFNDNCPTAVLEKDAVCSSSPSVVVSARTLNNRYTGPYTFALEDQPVKLPAVWSITTLNATSALLRAQEQIPPGVYHISLVLTDSQNNRCEMPRSLTLEVCQCDNRGICGTSYPTTSPGTRYGRPHSGRLGPAAIGLLLLGLLLLLLAPLLLLTCDCGAGSTGGVTGGFIPVPDGSEGTIHQWGIEGAHPEDKEITNICVPPVTANGADFMESSEVCTNTYARGTAVEGTSGMEMTTKLGAATESGGAAGFATGTVSGAASGFGAATGVGICSSGQSGTMRTRHSTGGTNKDYADGAISMNFLDSYFSQKAFACAEEDDGQEANDCLLIYDNEGADATGSPVGSVGCCSFIADDLDDSFLDSLGPKFKKLAEISLGVDGEGKEVQPPSKDSGYGIESCGHPIEVQQTGFVKCQTLSGSQGASALSTSGSVQPAVSIPDPLQHGNYLVTETYSASGSLVQPSTAGFDPLLTQNVIVTERVICPISSVPGNLAGPTQLRGSHTMLCTEDPCSRLI

SEQ ID NO: 100 - protein, 98 aa, Homo sapiens
MNQTAILICCLIFLTLSGIQGVPLSRTVRCTCISISNQPVNPRSLEKLEIIPASQFCPRVEIIATMKKKGEKRCLNPESKAIKNLLKAVSKERSKRSP

SEQ ID NO: 101 - protein, 482 aa, Homo sapiens
MAALMTPGTGAPPAPGDFSGEGSQGLPDPSPEPKQLPELIRMKRDGGRLSEADIRGFVAAVVNGSAQGAQIGAMLMAIRLRGMDLEETSVLTQALAQSGQQLEWPEAWRQQLVDKHSTGGVGDKVSLVLAPALAACGCKVPMISGRGLGHTGGTLDKLESIPGFNVIQSPEQMQVLLDQAGCCIVGQSEQLVPADGILYAARDVTATVDSLPLITASILSKKLVEGLSALVVDVKFGGAAVFPNQEQARELAKTLVGVGASLGLRVAAALTAMDKPLGRCVGHALEVEEALLCMDGAGPPDLRDLVTTLGGALLWLSGHAGTQAQGAARVAAALDDGSALGRFERMLAAQGVDPGLARALCSGSPAERRQLLPRAREQEELLAPADGTVELVRALPLALVLHELGAGRSRAGEPLRLGVGAELLVDVGQRLRRGTPWLRVHRDGPALSGPQSRALQEALVLSDRAPFAAPSPFAELVLPPQQ

SEQ ID NO: 102 - protein, 396 aa, Homo sapiens
MTPTLLLTVTVPRAAGSAGQRRAPGLPRSSGPAWAESRARPPRPRGLEPRHPPGSPALRPTDRTCSSSSAGVGGGVGGAQPGSVPLGQHLALERGRTLGHGRVGRRDPPPLGLLVNPRVAGVDGLDRGGRLDPAGIGQVLGLGVLGGAGRQRRALGGQALGLLAQVGIGAGHARGGRGAVGPAGQHRARLGAAVLRGTAGAPARRVGVVAERAASAACSLQRQRLHARRRVREQRGRVAREVRGRVIGRGREVQPVVGRRHKPALRRGRARVLGLLRRQQPVGVRHAAVRTRPGARARARVEAGLGVVAHELVLQERAGHGVAGPGHDLRARRVVGRGGQAVHVTAGVDPAGLFQKPLGKSRARSNHERLAFTRVLEPEVCCWKPPKYLVSIVSPV

SEQ ID NO: 103 - protein, 493 aa, Homo sapiens
MTCGFNSIGCGFRPGNFSCVSACGPRPSRCCITAAPYRGISCYRGLTGGFGSHSVCGGFRAGSCGRSFGYRSGGVCGPSPPCITTVSVNESLLTPLNLEIDPNAQCVKQEEKEQIKSLNSRFAAFIDKVRFLEQQNKLLETKLQFYQNRECCQSNLEPLFAGYIETLRREAECVEADSGRLASELNHVQEVLEGYKKKYEEEVALRATAENEFVALKKDVDCAYLRKSDLEANVEALIQEIDFLRRLYEEEIRILQSHISDTSVVVKLDNSRDLNMDCMVAEIKAQYDDIATRSRAEAESWYRSKCEEMKATVIRHGETLRRTKEEINELNRMIQRLTAEVENAKCQNSKLEAAVAQSEQQGEAALSDARCKLAELEGALQKAKQDMACLIREYQEVMNSKLGLDIEIATYRRLLEGEEHRLCEGVEAVNVCVSSSRGGVVCGDLCVSGSRPVTGSVCSAPCNGNLVVSTGLCKPCGQLNTTCGGGSCGQGRY

SEQ ID NO: 104 - protein, 271 aa, Homo sapiens
MAKVPDMFEDLKNCYSENEEDSSSIDHLSLNQKSFYHVSYGPLHEGCMDQSVSLSISETSKTSKLTFKESMVVVATNGKVLKKRRLSLSQSITDDDLEAIANDSEEEIIKPRSAPFSFLSNVKYNFMRIIKYEFILNDALNQSIIRANDQYLTAAALHNLDEAVKFDMGAYKSSKDDAKITVILRISKTQLYVTAQDEDQPVLLKEMPEIPKTITGSETNLLFFWETHGTKNYFTSVAHPNLFIATKQDYWVCLAGGPPSITDFQILENQA

SEQ ID NO: 105 - protein, 473 aa, Homo sapiens
MTTCSRQFTSSSSMKGSCGIGGGIGGGSSRISSVLAGGSCRAPSTYGGGLSVSSRFSSGGACGLGGGYGGGFSSSSSFGSGFGGGYGGGLGAGFGGGLGAGFGGGFAGGDGLLVGSEKVTMQNLNDRLASYLDKVRALEEANADLEVKIRDWYQRQRPSEIKDYSPYFKTIEDLRNKIIAATIENAQPILQIDNARLAADDFRTKYEHELALRQTVEADVNGLRRVLDELTLARTDLEMQIEGLKEELAYLRKNHEEEMLALRGQTGGDVNVEMDAAPGVDLSRILNEMRDQYEQMAEKNRRDAETWFLSKTEELNKEVASNSELVQSSRSEVTELRRVLQGLEIELQSQLSMKASLENSLEETKGRYCMQLSQIQGLIGSVEEQLAQLRCEMEQQSQEYQILLDVKTRLEQEIATYRRLLEGEDAHLSSQQASGQSYSSREVFTSSSSSSSRQTRPILKEQSSSSFSQGQSS

SEQ ID NO: 106 - protein, 462 aa, Homo sapiens
MSSHGNSLFLRESGQRLGRVGWLQRLQESLQQRALRMRLRLQTMTREHVLRFLRRNAFILLTVSAVVIGVSLAFALRPYQLSYRQIKYFSFPGELLMRMLQMLVLPLIVSSLVTGMASLDNKATGRMGMRAAVYYMVTTVIAVFIGILMVTIIHPGKGSKEGLHREGRIETIPTADAFMDLVRNMFPPNLVEACFKQFKTQYSTRLVTRTVVRTDNGSELGTSMPPLSSLENGTGLLENVTRALGTLQEVLSFEETVPVPGSANGINALGLVVFSVAFGLVIGGMKHKGRVLRDFFDSLNEAIMRLVGIIIWYAPVGILFLIAGKILEMEDMAVLGGQLGMYTLTVIVGLFVHAGGILPLIYFLITHRNPFPFIGGILQALITAMGTSSSSATLPITFRCLEEGLGVDRRITRFVLPVGATVNMDGTALYEALAAIFIAQVNNYELNLGQITTISITATAAS

SEQ ID NO: 107 - DNA, 21 bp, Homo sapiens
agtgcaagagctgttccgggc

SEQ ID NO: 108 - DNA, 21 bp, Homo sapiens
ggttgagtttaagccaatcca

SEQ ID NO: 109 - DNA, 20 bp, Homo sapiens
atcgaggacctgaggaacaa

SEQ ID NO: 110 - DNA, 21 bp, Homo sapiens
tctgccacctggtctgccaca

SEQ ID NO: 111 - DNA, 19 bp, Homo sapiens
aagcctgctgacgatgatg

SEQ ID NO: 112 - DNA, 19 bp, Homo sapiens
ctatgggcacgtcttccag

SEQ ID NO: 113 - DNA, 22 bp, Homo sapiens
tgaggagtgcaggctgaatggc

SEQ ID NO: 114 - DNA, 25 bp, Homo sapiens
tctggactacacattcaggaggcac

SEQ ID NO: 115 - DNA, 19 bp, Homo sapiens
accgctggagccagacgcc

SEQ ID NO: 116 - DNA, 20 bp, Homo sapiens
gggcctcaatggacccaccg

SEQ ID NO: 117 - DNA, 21 bp, Homo sapiens
gtggagagggactggcagagc

SEQ ID NO: 118 - DNA, 20 bp, Homo sapiens
tactcggcgccccagtcgaa

SEQ ID NO: 119 - DNA, 20 bp, Homo sapiens
tgctgacctaggcttgatga

SEQ ID NO: 120 - DNA, 20 bp, Homo sapiens
gggccagttcatgctcatac

SEQ ID NO: 121 - DNA, 20 bp, Homo sapiens
agtgaccagcacagccagcg

SEQ ID NO: 122 - DNA, 21 bp, Homo sapiens
gcgaggtaatgtatgcccttt

SEQ ID NO: 123 - DNA, 18 bp, Homo sapiens
ggacgaacctggtgatgc

SEQ ID NO: 124 - DNA, 24 bp, Homo sapiens
caatggctctgccactgctggaac

SEQ ID NO: 125 - DNA, 27 bp, Homo sapiens
gtcacagagagctggttctgaattgtc

SEQ ID NO: 126 - DNA, 22 bp, Homo sapiens
cgagaggccaatgctgggtagc

SEQ ID NO: 127 - DNA, 20 bp, Homo sapiens
ctgggcctttggcctgcctt

SEQ ID NO: 128 - DNA, 25 bp, Homo sapiens
gggacactcctcaattcctacgatc

SEQ ID NO: 129 - DNA, 50 bp, Homo sapiens
tcccacttggcaggggccgtcttgtccactcgtttctgtaaacatgggtg

SEQ ID NO: 130 - DNA, 19 bp, Homo sapiens
aagcctgctgacgatgatg

SEQ ID NO: 131 - DNA, 21 bp, Homo sapiens
gcgaggtaatgtatgcccttt

SEQ ID NO: 132 - DNA, 1720 bp, Homo sapiens
agttaggagggccccgccttccccagctgcatataaaggtctctggggttggaggcagccacagcacgctctcagccttcctgagcacctttccttctttcagccaactgctcactcgctcacctccctccttggcaccatgaccacctgcagccgccagttcacctcctccagctccatgaagggctcctgcggcatcggaggcggcatcgggggcggctccagccgcatctcctccgtcctggccggagggtcctgccgtgcccccagcacctacgggggcggcctgtctgtctcctctcgcttctcctctgggggagcctgcgggctggggggcggctatggcggtggcttcagcagcagcagcagctttggtagtggcttcgggggaggatatggtggtggccttggtgctggcttcggtggtggcttgggtgctggctttggtggtggttttgctggtggtgatgggcttctggtgggcagtgagaaggtgaccatgcagaacctcaatgaccgcctggcctcctacctggacaaggtgcgtgctctggaggaggccaacgccgacctggaagtgaagatccgtgactggtaccagaggcagcggcccagtgagatcaaagactacagtccctacttcaagaccatcgaggacctgaggaacaagatcattgcggccaccattgagaatgcgcagcccattttgcagattgacaatgccaggctggcagccgatgacttcaggaccaagtatgagcatgaactggccctgcggcagactgtggaggccgacgtcaatggcctgcgccgggtgttggatgagctgaccctggccaggactgacctggagatgcagatcgaaggcctgaaggaggagctggcctacctgaggaagaaccacgaggaggagatgcttgctctgagaggtcagaccggcggagatgtgaacgtggagatggatgctgcacctggcgtggacctgagccgcatcctgaatgagatgcgtgaccagtacgagcagatggcagagaaaaaccgcagagacgctgagacctggttcctgagcaagaccgaggagctgaacaaagaagtggcctccaacagcgaactggtacagagcagccgcagtgaggtgacggagctccggagggtgctccagggcctggagattgagctgcagtcccagctcagcatgaaagcatccctggagaacagcctggaggagaccaaaggccgctactgcatgcagctgtcccagatccagggactgattggcagtgtggaggagcagctggcccagctacgctgtgagatggagcagcagagccaggagtaccagatcttgctggatgtgaagacgcggctggagcaggagattgccacctaccgccgcctgctggagggcgaggatgcccacctttcctcccagcaagcatctggccaatcctattcttcccgcgaggtcttcacctcctcctcgtcctcttcgagccgtcagacccggcccatcctcaaggagcagagctcatccagcttcagccagggccagagctcctagaactgagctgcctctaccacagcctcctgcccaccagctggcctcacctcctgaaggcccgggtcaggaccctgctctcctggcgcagttcccagctatctcccctgctcctctgctggtggtgggctaataaagctgactttctggttgatgcaaaaa

SEQ ID NO: 133 - DNA, 1719 bp, Homo sapiens
gatagaccatgagcagccatggcaacagcctgttccttcgggagagcggccagcggctgggccgggtgggctggctgcagcggctgcaggaaagcctgcagcagagagcactgcgcacgcgcctgcgcctgcagaccatgaccctcgagcacgtgctgcgcttcctgcgccgaaacgccttcattctgctgacggtcagcgccgtggtcattggggtcagcctggcctttgccctgcgcccatatcagctcacctaccgccagatcaagtacttctcttttcctggagagcttctgatgaggatgctgcagatgctggtgttacctctcattgtctccagcctggtcacaggtatggcatccctggacaacaaggccacggggcggatggggatgcgggcagctgtgtactacatggtgaccaccatcatcgcggtcttcatcggcatcctcatggtcaccatcatccatcccgggaagggctccaaggaggggctgcaccgggagggccggatcgagaccatccccacagctgatgccttcatggacctgatcagaaatatgtttccaccaaaccttgtggaggcctgcttcaaacagttcaagacgcagtacagcacgagggtggtaaccaggaccatggtgaggacagagaacgggtctgagccgggtgcctccatgcctcctccattctcagtggagaacggaaccagcttcctggaaaatgtcactcgggccttgggtaccctgcaggagatgctgagctttgaggagactgtacccgtgcctggctccgccaatggcatcaacgccctgggcctcgtggtcttctctgtggcctttgggctggtcattggtggcatgaaacacaagggcagagtcctcagggacttcttcgacagcctcaatgaggctattatgaggctggtgggcatcattatctggtatgcacctgtgggcatcctgttcctgattgctgggaagattctggagatggaagacatggccgtcctggggggtcagctgggcatgtacaccctgaccgtcatcgtgggcctgttcctccatgccggcattgtccttcccctcatctacttcctcgtcactcaccggaaccccttccccttcattgggggcatgctacaagccctcatcaccgctatgggcacgtcttccagctcggcaacgctgcccatcaccttccgctgcctggaggagggcctgggtgtggaccgccgcatcaccaggttcgtcctgcccgtgggcgccacggtcaacatggatggcactgccctctacgaggccctggctgccatcttcattgctcaagttaacaactacgagctcaacctgggtcagatcacaaccatcagcatcacggccacagcagccagtgttggggctgctggcatcccccaggcgggtctggtcaccatggtcattgtgcttacgtcggtcggcttgcccacggaagacatcacgctcatcatcgccgtggactggttccttgaccggcttcgcacaatgaccaacgtactgggggactcaattggagcggccgtcatcgagcacttgtctcagcgggagctggagcttcaggaagctgagcttaccctccccagcctggggaaaccctacaagtccctcatggcacaggagaagggggcatcccggggacggggaggcaacgagagtgctatgtgaggggcctccagctctg

SEQ ID NO: 134 - DNA, 22 bp, Homo sapiens
actggtggcaggggcttctagc

SEQ ID NO: 135 - DNA, 28 bp, Homo sapiens
gccatctaaagtaactaaacccatagac

SEQ ID NO: 136 - DNA, 50 bp, Homo sapiens
gccatggccagtgcaagttctagcgctacaggtagcttctcctaccctga

SEQ ID NO: 137 - DNA, 50 bp, Homo sapiens; identical to SEQ ID NO: 129

SEQ ID NO: 138 - DNA, 50 bp, Homo sapiens
tctatttgaagcatgctctgtaagttgcttcctaacatccttggactgag

SEQ ID NO: 139 - DNA, 50 bp, Homo sapiens
tgattggtgcccagttagcctctgcaggatgtggaaacctccttccaggg

SEQ ID NO: 140 - DNA, 50 bp, Homo sapiens
gaaggctggctcatacattttcccagacaggaatttggctgccaacaggg

SEQ ID NO: 141 - DNA, 50 bp, Homo sapiens
cagtgggaaaggccaccctagaaagaagtccgctggcacccataggaagg

SEQ ID NO: 142 - DNA, 50 bp, Homo sapiens
cctgcagacaccggaggcctctgctgtggctgcccactggctgtgctcag

SEQ ID NO: 143 - DNA, 50 bp, Homo sapiens
tggttagactctggagtgactgggagtgggctagaaggggaccacctgtc

SEQ ID NO: 144 - DNA, 50 bp, Homo sapiens
caggtcttggtaggtgcctgcatctgtctgccttctggctgacaatcctg

SEQ ID NO: 145 - DNA, 50 bp, Homo sapiens
gctgagaggtccataataggcatgatcgacatgtttcacaaatacaccag

SEQ ID NO: 146 - DNA, 50 bp, Homo sapiens
gctgtggattacatcttgtgtgtgtcagagaaactgcagagaacctggag

SEQ ID NO: 147 - DNA, 50 bp, Homo sapiens
gacttccactgccatcctcccaaggggcccaaattctttcagtggctacc

SEQ ID NO: 148 - DNA, 50 bp, Homo sapiens
gctgcagctcgttcctcacctgcatgagagaagaatgaagagattcagag

SEQ ID NO: 149 - DNA, 50 bp, Homo sapiens
gcaggaacaacagatgcaggaacaggctgcacagctcagcacaacattcc

SEQ ID NO: 150 - DNA, 50 bp, Homo sapiens
gcatgacctggagccacggtctctcagtatctaaagtcctacacaaggcc

SEQ ID NO: 151 - DNA, 50 bp, Homo sapiens
ctacaggtcccctctgagccctctcaccttgtcctgtggaagaagcacag

SEQ ID NO: 152 - DNA, 50 bp, Homo sapiens
ggggcacaactcactactctgacaacaacagccctgcgagcaacatagtt

SEQ ID NO: 153 - DNA, 50 bp, Homo sapiens
ctgactgcccaggaatcaagaagtgctgtgaaggctcttgcgggatggcc

SEQ ID NO: 154 - DNA, 50 bp, Homo sapiens
agagttctgaccagcaccagataagcttcagtgctctcctttctttggcc

SEQ ID NO: 155 - DNA, 50 bp, Homo sapiens
cgcctgtgtgaaagctctggtctccctgaggagctaccatctgcaaatcg

SEQ ID NO: 156 - DNA, 50 bp, Homo sapiens
gctgcgtcataaggagacttctgtcttctccagaaggcaataccaacctg

SEQ ID NO: 157 - DNA, 50 bp, Homo sapiens
aaaacagcactgccttccgcgctgccccagcttgccccattccttgtccg

SEQ ID NO: 158 - DNA, 50 bp, Homo sapiens
tgaccgggagccgcggtctcgtgctatctggagtcctacacaaggccttt

SEQ ID NO: 159 - DNA, 50 bp, Homo sapiens
gtgtgctgttgagggaggtatcctgttgttcttactcactcttctcataa

SEQ ID NO: 160 - DNA, 50 bp, Homo sapiens
tgttcaagtcctagtctataggattggcagtttaaatgctttactccccc

SEQ ID NO: 161 - DNA, 50 bp, Homo sapiens
tgagtggaggcgaaccactgagccaaggagctggtagttacgttgtccgc

SEQ ID NO: 162 - DNA, 50 bp, Homo sapiens
gagggacccttggctcctgtgtctggtccacacaccacagaagcttgtat

SEQ ID NO: 163 - DNA, 50 bp, Homo sapiens
cctcctccaggtgcgaaggtccagctcagtggcacaagtgaaagcaatga

SEQ ID NO: 164 - DNA, 50 bp, Homo sapiens
gctggtgaaaccccgaagatcaacacgcttcaaactcagccccttggaac

SEQ ID NO: 165 - DNA, 50 bp, Homo sapiens
cgagtacacactattctgacggcagacctggttattgtgatgaagcgagg

SEQ ID NO: 166 - DNA, 50 bp, Homo sapiens
ctaggttcaccagttgagggacatttggattgttcccacttcttgggctg

SEQ ID NO: 167 - DNA, 50 bp, Homo sapiens
ctgattcctgcaagccagcagccaaaggagaataagacctctgaagaccc

SEQ ID NO: 168 - DNA, 50 bp, Homo sapiens
accacattctttggttcccaggagagccccattcccagcccctggtctcc

SEQ ID NO: 169 - DNA, 50 bp, Homo sapiens
tgctgctggaagcctccaaagtacttagtgtctattgtttcccctgtgtg

SEQ ID NO: 170 - DNA, 50 bp, Homo sapiens
gtggacacagatcccactggaagatcccctctcctgcccaagcacttcac

SEQ ID NO: 171 - DNA, 50 bp, Homo sapiens
cagcctggtcacctcctgaggaataaatgctgaacctcacaagccccatc

SEQ ID NO: 172 - DNA, 50 bp, Homo sapiens
gctggcttctgtagccacctgtcccttctatttttcagcgaaggtcagtg

SEQ ID NO: 173 - DNA, 50 bp, Homo sapiens
cctgtggcaagccagcaagatggccctggtgacagcaaaagaaactgcac

SEQ ID NO: 174 - DNA, 50 bp, Homo sapiens
acaggaagcaaactaagcccccgctgtaatgaaacaccttctctggagcc

SEQ ID NO: 175 - DNA, 50 bp, Homo sapiens
cagaaagggtgatctgtcccatttccagtgttcctggcaacctagctggc

SEQ ID NO: 176 - DNA, 50 bp, Homo sapiens
agaaactcgtggaggggctgtccgctctggtggtggacgttaagttcgga

SEQ ID NO: 177 - DNA, 50 bp, Homo sapiens
gtgttgtgaacccccacccaggcagtatccatgaaagcacaagtgactag

SEQ ID NO: 178 - DNA, 50 bp, Homo sapiens
ctcttgcagtgtccctgaatggcaagtgatgtaccttctgatgcagtctg

SEQ ID NO: 179 - DNA, 50 bp, Homo sapiens
ggacttcagccaacaggattcctgagctgaaatgcagatgaccacattca

SEQ ID NO: 180 - DNA, 50 bp, Homo sapiens
ccacatcgtcttccctgtcccaatcgaccagtgtatcgacggctgagtgc

SEQ ID NO: 181 - DNA, 50 bp, Homo sapiens
ccaggatgatcttctgtgctgggacagggactctgcctcttggagtttgg

SEQ ID NO: 182 - DNA, 50 bp, Homo sapiens
tggaagatcagacccagctccttacccttgtctgccagttgtaccagggc

SEQ ID NO: 183 - DNA, 50 bp, Homo sapiens
ctacaggcgcctgctggagggcgaggagcataggctgtgtgaaggtgttg

SEQ ID NO: 184 - DNA, 50 bp, Homo sapiens
ccccgtttatccatgtgtccattgacggccatctatgttgcttcttcggc

SEQ ID NO: 185 - DNA, 50 bp, Homo sapiens
taacttccaggagttcctcattctggtgataaagatgggcgtggcagccc

SEQ ID NO: 186 - DNA, 50 bp, Homo sapiens
gctcacttcgatgaggatgaacgctggacggatggtagcagtctagggat

SEQ ID NO: 187 - DNA, 50 bp, Homo sapiens
gcaactcatgaacttggccattctttgggtatgggacattcctctgatcc

SEQ ID NO: 188 - DNA, 50 bp, Homo sapiens
gctccaccttcatcttctcatcaaagcctaccatggatacacagggagct

SEQ ID NO: 189 - DNA, 50 bp, Homo sapiens
gatgagctttgtctacttcaaaagtttgtttgcttaccccttcagcctcc

SEQ ID NO: 190 - protein, 9 aa, Homo sapiens
SLLKFLAKV

SEQ ID NO: 191 - protein, 9 aa, Homo sapiens
MLLVFGIDV

SEQ ID NO: 192 - protein, 9 aa, Homo sapiens
KVTDLVQFL

SEQ ID NO: 193 - protein, 9 aa, Homo sapiens
GLYDGMEHL

SEQ ID NO: 194 - protein, 9 aa, Homo sapiens
ILILSIIFI

SEQ ID NO: 195 - protein, 9 aa, Homo sapiens
FLWGPRAHA

SEQ ID NO: 196 - protein, 9 aa, Homo sapiens
VIWEALNMM

SEQ ID NO: 197 - protein, 9 aa, Homo sapiens
KMSLLKFLA

SEQ ID NO: 198 - protein, 9 aa, Homo sapiens
FVLVTSLGL

SEQ ID NO: 199 - protein, 9 aa, Homo sapiens
LIFSEASEC

SEQ ID NO: 200 - protein, 9 aa, Homo sapiens
GMLSDVQSM

SEQ ID NO: 201 - protein, 9 aa, Homo sapiens
ILILILSII

SEQ ID NO: 202 - protein, 9 aa, Homo sapiens
GILILILSI

SEQ ID NO: 203 - protein, 8 aa, Homo sapiens
NMGLYDGM

SEQ ID NO: 204 - protein, 9 aa, Homo sapiens
QIACSSPSV

SEQ ID NO: 205 - protein, 9 aa, Homo sapiens
LIPSTPEEV

SEQ ID NO: 206 - protein, 9 aa, Homo sapiens
SMPKTGILI

SEQ ID NO: 207 - protein, 9 aa, Homo sapiens
IIFIEGYCT

SEQ ID NO: 208 - protein, 8 aa, Homo sapiens
WEALNMGL